(19) United States
(12) Patent Application Publication
(10) Pub. No.: US 2021/0345613 A1
(43) Pub. Date: Nov. 11, 2021

(54) HERBICIDAL PYRIMIDINE COMPOUNDS

(71) Applicant: BASF SE, Ludwigshafen am Rhein (DE)

(72) Inventors: Florian VOGT, Ludwigshafen (DE); Matthias WITSCHEL, Ludwigshafen (DE); Tobias SEISER, Limburgerhof (DE); Veronica LOPEZ CARRILLO, Ludwigshafen (DE); Thomas SEITZ, Ludwigshafen (DE); Gerd KRAEMER, Limburgerhof (DE); Trevor William NEWTON, Limburgerhof (DE)

(21) Appl. No.: 16/321,230

(22) PCT Filed: Jul. 11, 2017
(86) PCT No.: PCT/EP2017/067423

§ 371 Date: Jan. 28, 2019

(30) Foreign Application Priority Data
Jul. 28, 2016 (EP) 16181671.5

(51) Int. Cl. A01N 43/76; A01N 43/54

(52) U.S. Cl. CPC A01N 43/76; A01N 43/54

(57) ABSTRACT

The present invention relates to the pyrimidine compounds of formula (I),

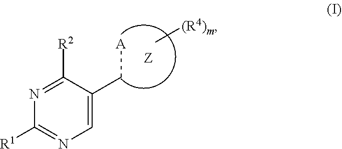

or their agriculturally acceptable salts or derivatives as herbicides, wherein the variables are defined according to the description, use of pyrimidine compounds of formula (I) as herbicide, compositions comprising them and their use as herbicides, i.e. for controlling harmful plants, and also a method for controlling unwanted vegetation which comprises allowing a herbicidal effective amount of at least one pyrimidine compounds of the formula (I) to act on plants, their seed and/or their habitat.

The present invention relates to pyrimidine compounds of the general formula (I) defined below and to their use as herbicides. Moreover, the invention relates to compositions for crop protection and to a method for controlling unwanted vegetation.

### DESCRIPTION

Compounds having a 5-phenyl pyrimidine moiety are known in the art. WO 2000/073278 describes such compounds being antagonists of the Neurokinin 1 receptor and thus having pharmaceutical properties.

In agriculture, there is a constant demand to develop novel active ingredients, which complement or outperform present methods of treatment regarding activity, selectivity and environmental safety.

These and further objects are achieved by pyrimidine compounds of formula (I), defined below, and by their agriculturally suitable salts.

Accordingly, the present invention provides the pyrimidine compounds of formula (I)

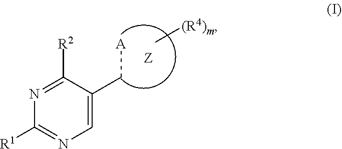

wherein
the dotted line ( [symbol] ) is a single bond or a double bond;
R¹ is C₁-C₆-alkyl, C₁-C₆-haloalkyl, HO—C₁-C₆-alkyl, C₃-C₆-alkenyl, C₃-C₆-haloalkenyl, C₂-C₆-alkynyl, C₃-C₆-haloalkynyl, C₁-C₆-alkoxy-C₁-C₆-alkyl, C₁-C₆-alkoxy, C₃-C₆-alkenyloxy, C₃-C₆-haloalkenyloxy, C₃-C₆-alkynyloxy, C₃-C₆-haloalkynyloxy, C₁-C₆-haloalkoxy, C₃-C₆-cycloalkoxy, C₃-C₆-halocycloalkoxy, C₃-C₆-cycloalkenyloxy, C₃-C₆-halocycloalkenyloxy, C₁-C₆-alkylthio, C₁-C₆-haloalkylthio, (C₁-C₆-alkyl)amino, di(C₁-C₆-alkyl)amino, C₁-C₆-alkylsulfinyl, C₁-C₆-alkylsulfonyl, C₃-C₆-cycloalkyl, C₃-C₆-cycloalkenyl, C₃-C₆-halocycloalkyl, C₃-C₆-halocycloalkenyl, [1-(C₁-C₆-alkyl)]-C₃-C₆-cycloalkyl, [1-(C₃-C₆-alkenyl)]-C₃-C₆-cycloalkyl, [1-(C₂-C₆-alkynyl)]-C₃-C₆-cycloalkyl, [1-(C₁-C₆-haloalkyl)]-C₃-C₆-cycloalkyl, [1-(C₃-C₆-haloalkenyl)]-C₃-C₆-cycloalkyl, [1-(C₃-C₆-haloalkynyl)]-C₃-C₆-cycloalkyl, C₃-C₆-cycloalkyl-C₁-C₆-alkyl, C₃-C₆-cycloalkyl-C₁-C₆-haloalkyl, C₃-C₆-cycloalkyl-C₁-C₆-alkoxy, C₃-C₆-cycloalkyl-C₁-C₆-haloalkoxy, phenyl, 5- or 6-membered heteroaryl, or 3- to 6-membered heterocyclyl; wherein the cyclic groups of R¹ are unsubstituted or substituted by R^{a}; R² is C₁-C₆-alkyl, C₁-C₆-haloalkyl, C₂-C₆-alkenyl, C₂-C₆-haloalkenyl, C₁-C₆-alkoxy-C₂-C₆-alkenyl, C₁-C₆-alkoxy-C₂-C₆-haloalkenyl, C₁-C₆-haloalkoxy-C₂-C₆-alkenyl, C₁-C₆-haloalkoxy-C₂-C₆-haloalkenyl, C₂-C₆-alkynyl, C₂-C₆-haloalkynyl, C₁-C₆-alkoxy-C₂-C₆-alkynyl, C₁-C₆-alkoxy-C₃-C₆-haloalkynyl, C₁-C₆-haloalkoxy-C₂-C₆-alkynyl, C₁-C₆-haloalkoxy-C₃-C₆-haloalkynyl, C₃-C₆-cycloalkyl, C₃-C₆-halocycloalkyl, C₃-C₆-cycloalkenyl, C₃-C₆-halocycloalkenyl, C₃-C₆-cycloalkyl-C₁-C₆-alkyl, C₃-C₆-cycloalkyl-C₁-C₆-haloalkyl, C₃-C₆-halocycloalkyl-C₁-C₆-alkyl, C₃-C₆-halocycloalkyl-C₁-C₆-haloalkyl, C₃-C₆-cycloalkenyl-C₁-C₆-alkyl, C₃-C₆-cycloalkenyl-C₁-C₆-haloalkyl, C₃-C₆-halocycloalkenyl-C₁-C₆-alkyl, C₃-C₆-halocycloalkenyl-C₁-C₆-haloalkyl, C₃-C₆-cycloalkyl-C₂-C₆-alkenyl, C₃-C₆-cycloalkyl-C₂-C₆-haloalkenyl, C₃-C₆-halocycloalkyl-C₂-C₆-alkenyl, C₃-C₆-halocycloalkyl-C₂-C₆-haloalkenyl, C₃-C₆-cycloalkenyl-C₂-C₆-alkenyl, C₃-C₆-cycloalkenyl-C₂-C₆-haloalkenyl, C₃-C₆-halocycloalkenyl-C₂-C₆-alkenyl, C₃-C₆-halocycloalkenyl-C₂-C₆-haloalkenyl, C₃-C₆-cycloalkyl-C₂-C₆-alkynyl, C₃-C₆-cycloalkyl-C₃-C₆-haloalkynyl, C₃-C₆-halocycloalkyl-C₂-C₆-alkynyl, C₃-C₆-halocycloalkyl-C₃-C₆-haloalkynyl, C₃-C₆-cycloalkenyl-C₂-C₆-alkynyl, C₃-C₆-cycloalkenyl-C₃-C₆-haloalkynyl, C₃-C₆-halocycloalkenyl-C₂-C₆-alkynyl, C₃-C₆-halocycloalkenyl-C₃-C₆-haloalkynyl, C₃-C₆-cycloalkyl-C₁-C₆-alkylidenyl, C₃-C₆-cycloalkyl-C₂-C₆-haloalkylidenyl, C₃-C₆-halocycloalkyl-C₁-C₆-alkylidenyl, C₃-C₆-halocycloalkyl-C₂-C₆-haloalkylidenyl, C₃-C₆-cycloalkenyl-C₁-C₆-alkylidenyl, C₃-C₆-cycloalkenyl-C₂-C₆-haloalkylidenyl, C₃-C₆-halocycloalkenyl-C₁-C₆-alkylidenyl, C₃-C₆-halocycloalkenyl-C₂-C₆-haloalkylidenyl, heterocyclyl-C₁-C₆-alkylidenyl, heterocyclyl-C₂-C₆-haloalkylidenyl, C₃-C₆-hydroxycycloalkyl-C₁-C₆-alkyl, C₃-C₆-hydroxycycloalkyl-C₁-C₆-haloalkyl, C₃-C₆-hydroxycycloalkenyl-C₁-C₆-alkyl, C₃-C₆-hydroxycycloalkenyl-C₁-C₆-haloalkyl, C₁-C₆-hydroxyalkyl, C₂-C₆-hydroxyhaloalkyl, C₃-C₆-hydroxyalkenyl, C₃-C₆-hydroxyhaloalkenyl, C₃-C₆-hydroxyalkynyl, C₄-C₆-hydroxyhaloalkynyl, C₃-C₆-hydroxycycloalkyl, C₃-C₆-hydroxyhalocycloalkyl, C₃-C₆-hydroxycycloalkenyl, C₃-C₆-hydroxyhalocycloalkenyl, C₃-C₆-cycloalkyl-C₁-C₆-hydroxyalkyl, C₃-C₆-cycloalkyl-C₂-C₆-hydroxyhaloalkyl, C₃-C₆-halocycloalkyl-C₁-C₆-hydroxyalkyl, C₃-C₆-halocycloalkyl-C₂-C₆-hydroxyhaloalkyl, C₃-C₆-cycloalkenyl-C₁-C₆-hydroxyalkyl, C₃-C₆-cycloalkenyl-C₂-C₆-hydroxyhaloalkyl, C₃-C₆-halo-cycloalkenyl-C₁-C₆-hydroxyalkyl, C₃-C₆-halocycloalkenyl-C₂-C₆-hydroxyhaloalkyl, C₃-C₆-cycloalkyl-C₃-C₆-hydroxyalkenyl, C₃-C₆-cycloalkyl-C₃-C₆-hydroxyhaloalkenyl, C₃-C₆-halo-cycloalkyl-C₃-C₆-hydroxyalkenyl, C₃-C₆-halocycloalkyl-C₃-C₆-hydroxyhaloalkenyl, C₃-C₆-cycloalkenyl-C₃-C₆-hydroxyalkenyl, C₃-C₆-cycloalkenyl-C₃-C₆-hydroxyhaloalkenyl, C₃-C₆-halocycloalkenyl-C₃-C₆-hydroxyalkenyl, C₃-C₆-halocycloalkenyl-C₃-C₆-hydroxyhaloalkenyl, C₃-C₆-cycloalkyl-C₃-C₆-hydroxyalkynyl, C₃-C₆-halocycloalkyl-C₃-C₆-hydroxyalkynyl, C₃-C₆-cycloalkenyl-C₃-C₆-hydroxyalkynyl, C₃-C₆-halocycloalkenyl-C₃-C₆-hydroxyalkynyl, C₃-C₆-cycloalkyl-C₂-C₆-hydroxyalkylidenyl, C₃-C₆-halocycloalkyl-C₂-C₆-hydroxyalkylidenyl, C₃-C₆-cycloalkenyl-C₂-C₆-hydroxyalkylidenyl, C₃-C₆-halocycloalkyl-C₂-C₆-hydroxyalkylidenyl, heterocyclyl-C₂-C₆-hydroxyalkylidenyl, hydroxycarbonyl-C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-hydroxyalkyl, C₁-C₆-haloalkoxycarbonyl-C₁-C₆-hydroxyalkyl, C₁-C₆-alkoxycarbonyl-C₁-C₆-haloalkyl, C₁-C₆-haloalkoxycarbonyl-C₁-C₆-haloalkyl, C₁-C₆-alkoxycarbonyl-C₁-C₆-alkyl, C₁-C₆-haloalkoxycarbonyl-C₁-C₆-alkyl, C₃-C₆-hydroxycycloalkyl-C₁-C₆-hydroxyalkyl, C₃-C₆-hydroxycycloalkenyl-C₁-C₆-hydroxyalkyl, C₃-C₆-hydroxycycloalkyl-C₃-C₆-hydroxyalkenyl, C₃-C₆-hydroxycycloalkenyl-C₃-C₆-hydroxyalkenyl, C₃-C₆-hydroxycycloalkyl-C₃-C₆-hydroxyalkynyl, C₃-C₆-hydroxycycloalkenyl-C₃-C₆-hydroxyalkenyl, C₂-C₆-dihydroxyalkyl, C₃-C₆-dihydroxy-haloalkyl, C₄-C₆-dihydroxyalkenyl, C₄-C₆-dihydroxyhaloalkenyl, C₄-C₆-dihydroxyalkynyl, C₅-C₆-dihydroxyhaloalkynyl, C₄-C₆-dihydroxycycloalkyl, C₄-C₆-dihydroxyhalocycloalkyl, C₄-C₆-dihydroxycycloalkenyl, C₄-C₆-dihydroxyhalocycloalkenyl, C₃-C₆-cycloalkyl-C₂-C₆-dihydroxyalkyl, C₃-C₆-halocycloalkyl-C₂-C₆-dihydroxyalkyl, C₃-C₆-cycloalkenyl-C₂-C₆-dihydroxyalkyl, C₃-C₆-halocycloalkenyl-C₂-C₆-dihydroxyalkyl, C₃-C₆-cycloalkyl-C₃-C₆-dihydroxy-alkenyl, C₃-C₆-halocycloalkyl-C₃-C₆-dihydroxyalkenyl, C₃-C₆-cycloalkenyl-C₃-C₆-dihydroxy-alkenyl, C₃-C₆-halocycloalkenyl-C₃-C₆-dihydroxyalkenyl, C₃-C₆-cycloalkyl-C₄-C₆-dihydroxy-alkynyl, C₃-C₆-halocycloalkyl-C₄-C₆-dihydroxyalkynyl, C₃-C₆-cycloalkenyl-C₄-C₆-dihydroxyalkynyl, C₃-C₆-halocycloalkyl-C₄-C₆-dihydroxyalkynyl, C₃-C₆-cycloalkyl-C₃-C₆-dihydroxy-alkylidenyl, C₃-C₆-halocycloalkyl-C₃-C₆-dihydroxyalkylidenyl, heterocyclyl-C₃-C₆-dihydroxy-alkylidenyl, hydroxycarbonyl-C₂-C₆-dihydroxyalkyl, hydroxycarbonyl-C₃-C₆-dihydroxy-haloalkyl, C₁-C₆-alkoxycarbonyl-C₂-C₆-dihydroxyalkyl, C₁-C₆-haloalkoxycarbonyl-C₂-C₆-dihydroxyalkyl, C₁-C₆-haloalkoxycarbonyl-C₃-C₆-dihydroxyhaloalkyl, C₃-C₆-dihydroxycycloalkyl-C₁-C₆-alkyl, C₃-C₆-dihydroxycycloalkyl-C₁-C₆-haloalkylC₃-C₆-dihydroxycycloalkyl-C₂-C₆-alkenyl, C₃-C₆-dihydroxycycloalkyl-C₂-C₆-haloalkenyl, C₃-C₆-dihydroxycycloalkyl-C₂-C₆-alkynyl, C₃-C₆-dihydroxycycloalkyl-C₃-C₆-haloalkynyl, C₁-C₆-alkylcarbonyl-C₁-C₆-alkyl, C₁-C₆-haloalkylcarbonyl-C₁-C₆-alkyl, C₁-C₆-alkylcarbonyl-C₁-C₆-haloalkyl, C₁-C₆-haloalkylcarbonyl-C₁-C₆-haloalkyl, hydroxycarbonyl-C₂-C₆-alkenyl, hydroxycarbonyl-C₂-C₆-haloalkenyl, C₁-C₆-alkoxycarbonyl-C₂-C₆-alkenyl, C₁-C₆-haloalkoxycarbonyl-C₂-C₆-alkenyl, C₁-C₆-alkoxycarbonyl-C₂-C₆-haloalkenyl, C₁-C₆-haloalkoxycarbonyl-C₂-C₆-haloalkenyl, hydroxycarbonyl-C₂-C₆-alkynyl, hydroxycarbonyl-C₃-C₆-haloalkynyl, C₁-C₆-alkoxy-carbonyl-C₂-C₆-alkynyl, C₁-C₆-haloalkoxycarbonyl-C₂-C₆-alkynyl, C₁-C₆-alkoxycarbonyl-C₃-C₆-haloalkynyl, C₁-C₆-haloalkoxycarbonyl-C₃-C₆-haloalkynyl, C₁-C₆-cyanoalkyl, C₂-C₆-cyanohaloalkyl, C₁-C₆-dicyanoalkyl, C₂-C₆-dicyanohaloalkyl, di(hydroxycarbonyl)-C₁-C₆-alkyl, di(hydroxycarbonyl)-C₁-C₆-haloalkyl, di(C₁-C₆-alkoxycarbonyl)-C₁-C₆-alkyl, di(C₁-C₆-haloalkoxycarbonyl)-C₁-C₆-alkyl, di(C₁-C₆-alkoxycarbonyl)-C₁-C₆-haloalkyl, di(C₁-C₆-haloalkoxycarbonyl)-C₁-C₆-haloalkyl, di(C₁-C₆-alkoxyl)phosphoryl-C₁-C₆-alkyl, di(C₁-C₆-haloalkoxyl) phosphoryl-C₁-C₆-alkyl, di(C₁-C₆-alkoxyl)phosphoryl-C₁-C₆-haloalkyl, di(C₁-C₆-haloalkoxyl)phosphoryl-C₁-C₆-haloalkyl, phosphoryl-C₁-C₆-alkyl, phosphoryl-C₁-C₆-haloalkyl, di[di(C₁-C₆-alkoxyl)phosphoryl-)]C₁-C₆-alkyl, di[di(C₁-C₆-haloalkoxyl)phosphoryl-)]C₁-C₆-alkyl, di[di(C₁-C₆-alkoxyl)phosphoryl-)]C₁-C₆-haloalkyl, di[di(C₁-C₆-haloalkoxyl)phosphoryl-)]C₁-C₆-haloalkyl, diphosphoryl-C₁-C₆-alkyl, diphosphoryl-C₁-C₆-haloalkyl, C₁-C₆-alkylthio-C₁-C₆-alkyl, C₁-C₆-haloalkylthio-C₁-C₆-alkyl, C₁-C₆-alkylthio-C₁-C₆-haloalkyl, C₁-C₆-haloalkylthio-C₁-C₆-haloalkyl, C₁-C₆-alkylsulfinly-C₁-C₆-alkyl, C₁-C₆-haloalkylsulfinly-C₁-C₆-alkyl, C₁-C₆-alkylsulfinly-C₁-C₆-haloalkyl, C₁-C₆-haloalkylsulfinly-C₁-C₆-haloalkyl, C₁-C₆-alkylsulfonyl-C₁-C₆-alkyl, C₁-C₆-haloalkylsulfonyl-C₁-C₆-alkyl, C₁-C₆-haloalkylsulfonyl-C₁-C₆-haloalkyl, phenyl, 5- or 6-membered heteroaryl, 3- to 6-membered heterocyclyl;
   wherein hydroxy groups of R² are unsubstituted or substituted by R^{b};
   cyclic groups of R² are unsubstituted or substituted by R^{c}; and acyclic aliphatic groups of R² are unsubstituted or substituted by R^{d}.
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
   R^{c} is halogen, CN, NO₂, C₁-C₆-alkyl, C₁-C₆-haloalkyl, OH, C₁-C₆-alkoxy or C₁-C₆-haloalkoxy, C₁-C₆-alkylthio, C₁-C₆-alkylsulfinyl, or C₁-C₆-alkylsulfonyl;
   R^{d} is phenyl, 5- or 6-membered heteroaryl, or 3- to 6-membered heterocyclyl; wherein the substituent R^{d} is unsubstituted or substituted by R^{e};
   R^{e} is halogen, CN, NO₂, C₁-C₆-alkyl, C₁-C₆-haloalkyl, OH, C₁-C₆-alkoxy or C₁-C₆-haloalkoxy, C₁-C₆-alkylsulfonyl;
A is CR³ or NR^{3A};
Z is a 5 or 6 membered heteroaryl ring comprising A;
R³ is halogen, CN, NO₂, CHO, C₁-C₆-alkyl, C₁-C₆-haloalkyl, C₁-C₆-alkylcarbonyl, C₃-C₆-alkenyl, C₃-C₆-haloalkenyl, C₂-C₆-alkynyl, C₃-C₆-haloalkynyl, C₁-C₆-alkoxy, C₁-C₆-haloalkoxy, C₃-C₆-alkenyloxy, C₃-C₆-haloalkenyloxy, C₃-C₆-alkynyloxy, C₃-C₆-haloalkynyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
   wherein the cyclic groups of R³ are unsubstituted or substituted by substituents R^{a};
R^{3A} is H, C₁-C₆-alkyl, C₁-C₆-haloalkyl, C₁-C₆-alkylcarbonyl, C₃-C₆-alkenyl, C₃-C₆-haloalkenyl, C₂-C₆-alkynyl, C₃-C₆-haloalkynyl, C₁-C₆-alkoxy, C₁-C₆-haloalkoxy, C₃-C₆-alkenyloxy, C₃-C₆-haloalkenyloxy, C₃-C₆-alkynyloxy, C₃-C₆-haloalkynyloxy, C₁-C₆-alkoxy-C₁-C₆-alkoxy, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl) oxy, or phenyl;
   wherein the cyclic groups of R^{3A} are unsubstituted or substituted by R^{a};
R⁴ is halogen, CN, NO₂, CHO, C₁-C₆-alkyl, C₁-C₆-haloalkyl, C₁-C₆-alkylcarbonyl, C₃-C₆-alkenyl, C₃-C₆-haloalkenyl, C₃-C₆-alkenyl, C₃-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
   wherein the cyclic groups of R⁴ are unsubstituted or substituted by R^{a};
   R^{a} is halogen, CN, NO₂, C₁-C₆-alkyl, C₁-C₆-haloalkyl, C₁-C₆-alkoxy, or C₁-C₆-haloalkoxy;
m is 0, 1, 2, or 3;
   including agriculturally acceptable salts or derivatives of compounds of formula (I) having an acidic functionality.

The present invention also provides use of the pyrimidine compounds of formula (I) as described herein including agriculturally acceptable salts or derivatives of compounds of formula (I) having an acidic functionality, as herbicide.

The pyrimidine compounds of formula (I) according to the invention can be prepared by standard processes of organic chemistry, e.g. by the following processes:

Process A:

The pyrimidines of formula (II) can be obtained by reacting respective pyrimidines of formula (I), R²═CH₃, (prepared analogous to known procedures like e.g. in WO 2013186229) with base and an electrophile, e.g. a carbonyl compound (III):

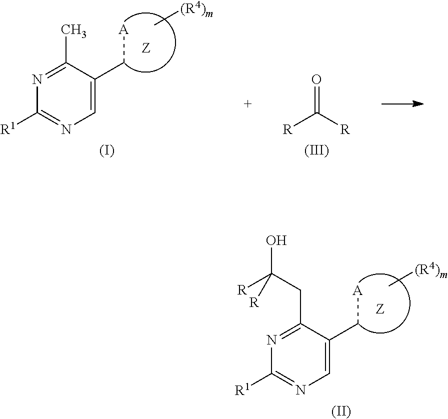

The reaction of the pyrimidine (1) with the electrophile (III), with R independent of each other equals hydrogen, alkyl, cycloalkyl, halocycloalkyl, haloalkyl, cycloalkenyl, halocycloalkenyl, alkenyl, haloalkenyl, alkynyl, phenyl, heterocyclyl, heteroaryl or both R form together a carbocycle or a heterocycle, is usually carried out at temperatures of from −100° C. to the boiling point of the reaction mixture, preferably from −80° C. to 20° C., particularly preferably from −80° C. to −20° C., in an inert organic solvent in the presence of a base.

Suitable solvents are aliphatic hydrocarbons, such as pentane, hexane, cyclohexane and mixtures of C₅-C₅-alkanes, aromatic hydrocarbons, such as toluene, o-, m- and p-xylene, ethers, such as diethyl ether, diisopropyl ether, tert-butyl methyl ether (TBME), dioxane, anisole and tetrahydrofuran (THF), and also dimethyl sulfoxide (DMSO), dimethylformamide (DMF) and N,N-dimethylacetamide (DMAC), particularly diethyl ether, dioxane and THF.

It is also possible to use mixtures of the solvents mentioned.

Suitable bases are, in general, inorganic compounds, such as alkali metal and alkaline earth metal anhydrides, such as lithium hydride (LiH), sodium hydride (NaH), potassium hydride (KH) and calcium hydride (CaH), alkali metal amides, such as lithium hexamethyidisilazide (LHMDS) and lithium diisopropylamide (LDA), organometallic compounds, in particular alkali metal alkyls, such as methyllithium (MeLi), butyllithium (BuLi) and phenyllithium (PhLi), and also alkali metal and alkaline earth metal alkoxides, such as sodium methoxide (NaOCH₃), sodium ethoxide (NaOC₂H₅), potassium ethoxide (KOC₂H₅), potassium tert-butoxide (tBuOK), potassium tert-pentoxide and dimethoxymagnesium, moreover organic bases, e.g. tertiary amines, such as trimethylamine (TMA), triethylamine (TEA), diisopropylethylamine (DIPEA) and N-methylpiperidine, pyridine, substituted pyridines, such as collidine, lutidine and 4-dimethylaminpyridine, and also bicyclic amines. Particular preference is given to NaH, LHMDS and lithium diisopropylamide (LDA).

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile, based on the pyrimidine compounds (1).

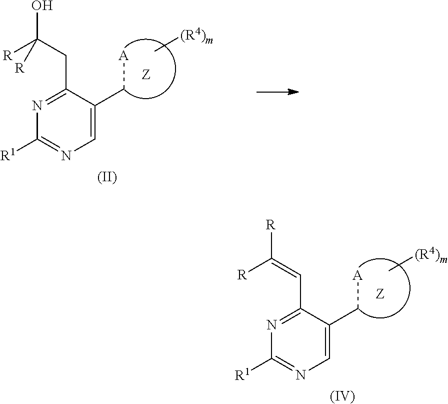

The elimination of the alcohol of the pyrimidine (II) is usually carried out at temperatures from −100° C. to the boiling point of the reaction mixture, preferably from 0° C. to 120° C., particularly preferably from 20° C. to 100° C., in an inert solvent optionally in the presence of an acid.

Suitable solvents are aliphatic hydrocarbons, such as pentane, hexane, cyclohexane and mixtures of C₅-C₅-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably toluene and o-xylene.

It is also possible to use mixtures of the solvents mentioned.

Suitable acids are inorganic acids, such as HCl, HBr, sulfuric acid; organic acids p-toluenesulfonic acid, benzene sulfonic acid, pyridinium p-toluol sulfonic acid, methanesulfonic acid, acetic acid; preferably p-toluenesulfonic acid and HCl.

The acids are generally employed in equimolar amounts; however, they can also be employed in catalytic amounts, in excess or, if appropriate, as solvents.

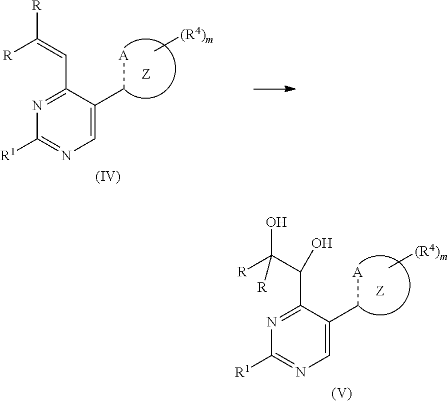

The oxidation of the olefin (IV) to the diol (V) is usually carried out at temperatures of from −100° C. to the boiling point of the reaction mixture, preferably from 0° C. to 120° C., particularly preferably from 20° C. to 100° C., in an inert solvent.

The reaction may in principle be carried out in substance. However, preference is given to reacting the pyrimidines (IV) with the oxidant in an organic solvent with or without water as co-solvent.

Suitable in principle are all solvents which are capable of dissolving the pyrimidines (IV) and the oxidant at least partly and preferably fully under the reaction conditions.

Suitable solvents are aliphatic hydrocarbons, such as pentane, hexane, cyclohexane and mixtures of C₅-C₅-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably TBME, THF It is also possible to use mixtures of the solvents mentioned.

Suitable oxidants are e.g. potassium permanganate, potassium perruthenate, osmium tetroxide and other osmium salts, like potassium osmate. The oxidant can be used in equimolar amounts or in catalytic amounts together with a reoxidant like N-methylmorpholine-N-oxide or potassium hexacyanoferrate in stochiometric amounts or in excess.

Process B:

The halopyrimidines (VI) are known or can be prepared by known procedures (X=Cl, Br, I).

The boronic acids or esters required for the preparation of pyrimidine compounds of formula (VII) are commercially available, known from literature or can easily prepared analogously to published procedures (e.g. Kamei et al. Tetrahedron Lett. 2014, 55, 4245-4247).

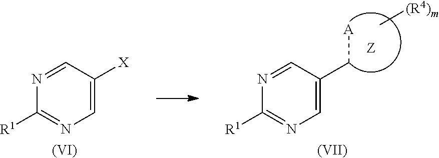

The pyrimidine compounds of formula (VII) can obtained by reacting phenyl boronic acids or esters with halides of formula (VI) in which X equals Cl, Br, or I in presence of a base and a catalyst in analogy to WO 2014202493.

The reaction may in principle be carried out in substance. However, preference is given to reacting the pyrimidines (VI) with the boronic acid or ester in an organic solvent with or without water as co-solvent.

Suitable in principle are all solvents which are capable of dissolving the pyrimidines (VI) and the boronic acid or ester at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, DMAC, DMI, N,N′-dimethylpropylene urea (DMPU), DMSO and 1-methyl-2 pyrrolidinone (NMP).

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂); alkali metal and alkaline earth metal acetates such as sodium acetate or potassium acetate.

The term base as used herein also includes mixtures of two or more, preferably two of the above bases. Particular preference is given to the use of one base.

The bases are used preferably from 1 to 10 equivalents based on the pyrimidine (VI), more preferably from 1.0 to 5.0 equivalents based on the pyrimidine (VI), most preferably from 1.2 to 2.5 equivalents based on the pyrimidine (VI).

It may be advantageous to add the base offset over a period of time.

The reaction of the pyrimidines (VI) with the phenyl boronic acid or ester is carried out in the presence of a catalyst. Examples of suitable catalysts include e.g., palladium based catalysts like, e.g., Palladium(II)acetate, tetrakis(triphenylphosphine)palladium(0), bis(triphenylphosphine) palladium(II)chloride, or (1,1,-bis(diphenylphosphino)ferrocene)-dichloropalladium(II), and optionally suitable additives such as, e.g., phosphines like, e.g., P(o-tolyl)3, triphenylphosphine, or BINAP (2,2′-Bis(diphenylphospino)-1,1′-binaphthyl).

The amount of catalyst is usually 0.01 to 10 mol % (0.0001 to 0.1 equivalents) based on the pyrimidine (VI).

The pyrimidine compounds of formula (VIII) can be obtained by reacting respective pyrimidines of formula (VII) with base and an electrophile, e.g. a carbonyl compound (III):

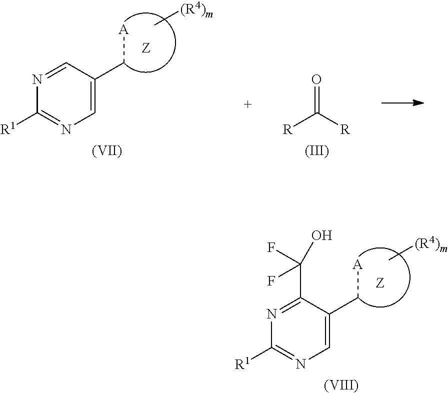

The reaction of the pyrimidine (VII) with the electrophile (III), with R independent of each other equals hydrogen, alkyl, cycloalkyl, halocycloalkyl, haloalkyl, cycloalkenyl, halocycloalkenyl, alkenyl, haloalkenyl, alkynyl, phenyl, heterocyclyl, heteroaryl or both R form together a carbocycle or a heterocycle, is usually carried out at temperatures from −100° C. to the boiling point of the reaction mixture, preferably from −80° C. to 20° C., particularly preferably from −80° C. to −20° C., in an inert organic solvent in the presence of a base.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also dimethyl sulfoxide, DMF and DMAC, particularly preferably diethyl ether, dioxane and THF.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile, based on the pyrimidine (VII).

Process C:

The pyrimidine compounds of formula (IX) can be obtained by reacting respective aminoketones of formula (X) with base and amidine (XI):

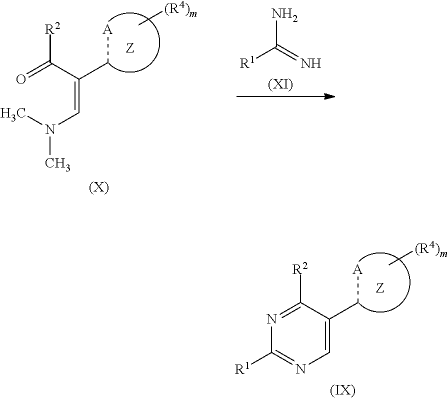

The reaction of the aminoketone (X) with the amidine (XI) is usually carried out at temperatures of from −100° C. to the boiling point of the reaction mixture, preferably from 20° C. to the boiling point, particularly preferably from 40° C. to 120° C., in an inert organic solvent in the presence of a base.

The reaction may in principle be carried out in substance. However, preference is given to reacting the aminoketones (X) with the amidine (XI) in an organic solvent.

Suitable in principle are all solvents which are capable of dissolving the aminoketones (X) with the amidine (XI) at least partly and preferably fully under the reaction conditions.

Examples of suitable solvents are aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, TBME, dioxane, anisole and THF, esters such as ethyl acetate and butyl acetate; nitriles such as acetonitrile and propionitrile, alcohols such as methanol, ethanol, n-propanol, isopropanol, n-butanol and tert.-butanol, as well as dipolar aprotic solvents such as sulfolane, dimethylsulfoxide, DMF, DMAC, DMIDMI, DMPU, DMSO and NMP.

Preferred solvents are alcohols such as methanol and ethanol.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaOCH₃, NaOC₂H₅, KOC₂H₅, potassium tert-butoxide and potassium tert-pentoxide.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the amidine (XI), based on the aminoketone (X).

Amidines (XI) known from literature or commercial available.

Preparation of Compound (X)

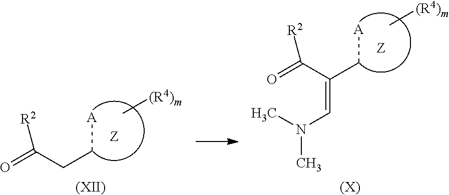

The aminoketones (X) are prepared from the corresponding ketones (XII) with N,N-DMF dimethyl acetal (CAS 4637-24-5). The reaction is usually carried out at temperatures from −100° C. to the boiling point of the reaction mixture, preferably from 20° C. to 160° C., particularly preferably from 50° C. to 130° C. The reaction can optionally be catalyzed be an acid.

The reaction may be carried out in substance or in an organic solvent. Suitable in principle are all solvents which are capable of dissolving the ketones (XII) and N,N-DMF dimethyl acetal (CAS 4637-24-5) at least partly and preferably fully under the reaction conditions.

Examples of suitable solvents are aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, TBME, dioxane, anisole and THF, esters such as ethyl acetate and butyl acetate; nitriles such as acetonitrile and propionitrile, as well as dipolar aprotic solvents such as sulfolane, DMSO, DMF, DMAC, DMIDMI, DMPU, DMSO and NMP; preferably DMF dimethyl acetal is used as solvent.

It is also possible to use mixtures of the solvents mentioned.

Suitable acids are inorganic acids, such as HCl, HBr, sulfuric acid; organic acids p-toluenesulfonic acid, benzene sulfonic acid, pyridinium p-toluol sulfonic acid, methanesulfonic acid, acetic acid; preferably p-toluenesulfonic acid and HCl. Most preferred, no acid is used.

The acids are generally employed in equimolar amounts; however, they can also be employed in catalytic amounts, in excess or, if appropriate, as solvents.

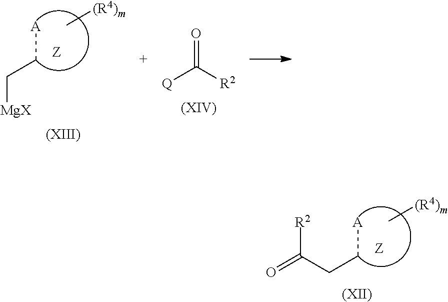

The ketones (XII) are prepared by reacting Grignard-reagent (XIII) with a carbonyl electrophile (XIV) (e.g. an acid halide (Q=F, Cl or Br) or a Weinreb-Amide (Q=N(Me)OMe)). The reaction is usually carried out at temperatures of from −100° C. to the boiling point of the reaction mixture, preferably from −80° C. to 60° C., particularly preferably from −80° C. to 20° C., in an inert solvent.

Suitable in principle are all solvents which are capable of dissolving the Grignard-reagent (XIII) and the carbonyl-electrophile (XIV) at least partly and preferably fully under the reaction conditions.

Examples of suitable solvents are aliphatic aromatic hydrocarbons such as benzene, chlorobenzene, toluene, cresols, o-, m- and p-xylene, ethers such as diethyl ether, diisopropyl ether, TBME, dioxane, anisole and THF, esters such as ethyl acetate and butyl acetate; nitriles such as acetonitrile and propionitrile, as well as dipolar aprotic solvents such as sulfolane, DMSO, DMF, DMAC, DMIDMI, DMPU, DMSO and NMP. Preferred solvents are ethers such as TBME or TH F.

It is also possible to use mixtures of the solvents mentioned.

The Grignard-reagents (XIII) are either commercial available or can be prepared from the corresponding halides by known methods.

The carbonyl electrophiles (XIV) are either commercial available or can be prepared from the corresponding carboxylic acid or carboxylic ester by known methods.

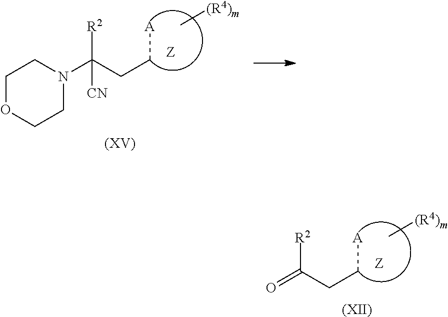

Ketones (XII) can as well be prepared from morpholinonitriles (XV) as described in the literature (European Journal of Organic Chemistry 2013, 36, 8083)

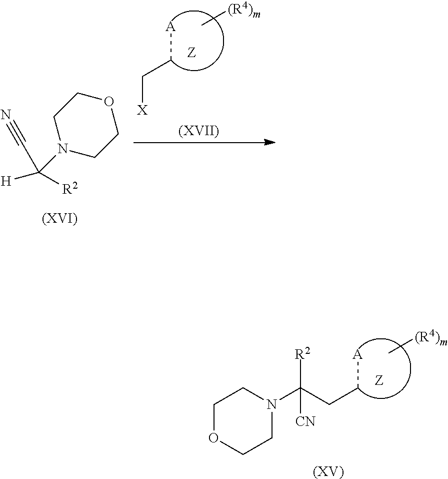

The morpholinonitriles (XV) are prepared from morpholinonitriles (XVI) and halides (XVII) in the presence of a base. The reaction is usually carried out at temperatures of from −100° C. to the boiling point of the reaction mixture, preferably from −80° C. to 60° C., particularly preferably from −50° C. to 20° C., in an inert organic solvent in the presence of a base.

Suitable in principle are all solvents which are capable of dissolving the morpholinonitriles (XVI) and the halides (XVII) at least partly and preferably fully under the reaction conditions.

Examples of suitable solvents are aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, TBME, dioxane, anisole and THF, esters such as ethyl acetate and butyl acetate; nitriles such as acetonitrile and propionitrile, as well as dipolar aprotic solvents such as sulfolane, DMSO, DMF, DMAC, DMIDMI, DMPU, DMSO and NMP. Preferred solvents are dipolar aprotic solvents such as sulfolane, dimethylsulfoxide, DMF, DMAC, DMIDMI, DMPU, DMSO and NMP.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the halide (XVII), based on the morpholinonitrile (XVI).

Halides (XVII) are commercially available and can easily prepared by methods known to a person skilled in the art.

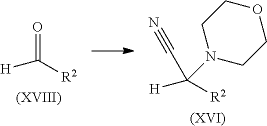

Morpholinonitriles (XVI) are prepared from the corresponding aldehydes (XVIII) as described in the literature (WO 2009/013462).

Aldehydes (XVIII) are commercial available.

Process D:

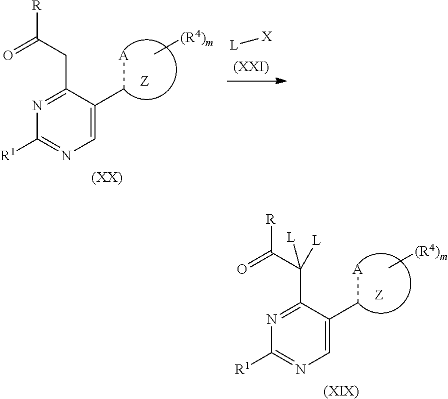

Pyrimidine compounds (XIX), with R equals alkyl, haloalkyl, alkoxy, haloalkoxy, X is a leaving group and L is halogen, alkyl, haloalkyl, alkenyl and alkynyl, can be obtained by reacting respective pyrimidine compounds of formula (XX) with base and an electrophile (XXI).

Electrophile (XXI) can be an alkyl-, alkenyl- or alkynyl-halide, e.g. methyl iodide, allyl bromide or propargyl bromide, or a halogenating agent, e.g. C₂, Br₂, I₂, NCS (N-Chlorosuccinimide), NBS (N-Bromosuccinimide), NIS (N-lodosuccinimide), NFSI (N-Fluorobenzenesulfonimide), Selectfluor (1-Chloromethyl-4-fluoro-1,4-diazoniabicyclo[2.2.2]octane bis(tetrafluoroborate)).

The reaction of the pyrimidine (XX) with the electrophile is usually carried out at temperatures of from −100° C. to the boiling point of the reaction mixture, preferably from −80° C. to 80° C., particularly preferably from −80° C. to 30° C., in an inert organic solvent in the presence of a base.

Suitable in principle are all solvents which are capable of dissolving the pyrimidine (XX) and the electrophile (XXI) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, nitriles such as acetonitrile and propionitrile, as well as dipolar aprotic solvents such as sulfolane, dimethylsulfoxide, DMF, N,N-DMAC (DMAC), DMIDMI, DMPU, DMSO and NMP.

Preferred solvents are ethers such as diethyl ether, diisopropyl ether, TBME, dioxane, anisole and THF and dipolar aprotic solvents such as sulfolane, dimethylsulfoxide, DMF, DMAC, DMIDMI, DMPU, DMSO and NMP.

More preferred solvents are ethers such as diethyl ether, diisopropyl ether, TBME, dioxane, anisole and THF.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile (XXI), based on the pyrimidine (XX).

The pyrimidine compounds of formula (XX) can be obtained by reacting respective pyrimidines of formula (I), R²═CH₃, (prepared analogous to known procedures like e.g. in WO 2013186229) with base and an electrophile (XXII), e.g. a dialkylcarbonate (X=R=alkoxy), an alkyl chloroformiate (X=halogene, R=alkoxy) or an acid halide (X=halogene, R=alkyl or haloalkyl):

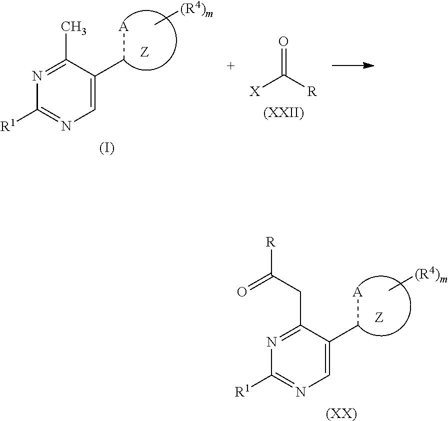

The reaction of the pyrimidine (1) with the electrophile (XXII) is usually carried out at temperatures of from −100° C. to the boiling point of the reaction mixture, preferably from −80° C. to 80° C., particularly preferably from −80° C. to 30° C., in an inert organic solvent in the presence of a base.

Suitable in principle are all solvents which are capable of dissolving the pyrimidine (1) and the electrophile (XXII) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, DMSO, DMF, DMAC, DMIDMI, DMPU, DMSO and NMP.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile (XXII), based on the pyrimidine (1).

Process E:

The pyrimidines of formula XXIII) can be obtained by reacting respective pyrimidines of formula (XXIV) with boronic acids/esters of formula (XXV):

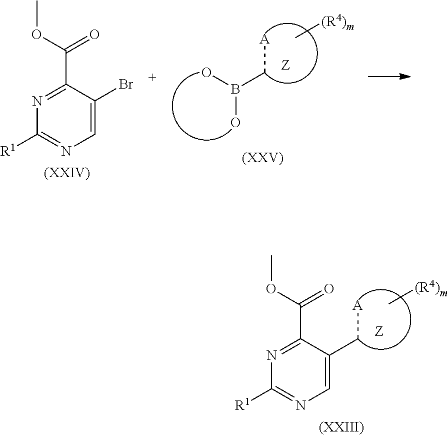

The reaction of pyrimidines (XXIV) with boronic acids/esters (XXV) is usually carried out from 0° C. to the boiling point of the reaction mixture, preferably from 15° C. to 110° C., particularly preferably from 40° C. to 100° C., in an inert organic solvent in the presence of a base and a catalyst.

The reaction may in principle be carried out in substance. However, preference is given to reacting the pyrimidines (XXIV) with the boronic acids/esters (XXV) in an organic solvent with or without water as co-solvent.

Suitable in principle are all solvents which are capable of dissolving the pyrimidines (XXIV) and the boronic acids (XXV) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, DMAC, DMIDMI, DMPU, DMSO and NMP.

Preferred solvents are ethers such as diethyl ether, diisopropyl ether, TBME, dioxane, anisole and THF and dipolar aprotic solvents such as sulfolane, DMSO, DMF, N,N-DMAC (DMAC), DMIDMI, DMPU, DMSO and NMP.

More preferred solvents are ethers such as diethyl ether, diisopropyl ether, TBME, dioxane, anisole and THF.

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂); alkali metal and alkaline earth metal acetates such as sodium acetate or potassium acetate.

The term base as used herein also includes mixtures of two or more, preferably two of the above bases. Particular preference is given to the use of one base.

The bases are used preferably from 1 to 10 equivalents based on the pyrimidine (XXIV), more preferably from 1.0 to 5.0 equivalents based on the pyrimidine (XXIV), most preferably from 1.2 to 2.5 equivalents based on the pyrimidine (XXIV).

It may be advantageous to add the base offset over a period of time.

The reaction of the pyrimidines (XXIV) with the boronic acids/esters (XXV) is carried out in the presence of a catalyst. Examples of suitable catalysts include e.g., palladium based catalysts like, e.g., palladium(II)acetate, tetrakis(triphenylphosphine)-palladium(0), bis(triphenylphosphine) palladium(II)chloride or (1,1,-bis(diphenylphosphino)-ferrocene)-dichloropalladium(II), and optionally suitable additives such as, e.g., phosphines like, e.g., P(o-tolyl)₃, triphenylphosphine or BINAP (2,2′-Bis(diphenylphospino)-1,1′-binaphthyl).

The amount of catalyst is usually 0.01 to 20 mol % (0.0001 to 0.2 equivalents) based on the pyrimidine (XXIV).

The halopyrimidines XXIV are known from the literature (e.g. WO 2011154327), are commercially available or can be prepared by known procedures.

The boronic acids/esters XXV required for the preparation of pyrimidines of formula (XVII) are commercially available, known from literature or can easily prepared analogously to published procedures (e.g. Kamei et al. Tetrahedron Lett. 2014, 55, 4245-4247).

The pyrimidines of formula (XXVI) can be obtained by reacting respective pyrimidines of formula (XXIII) with a reducing agent such as LAH or DIBAIH.

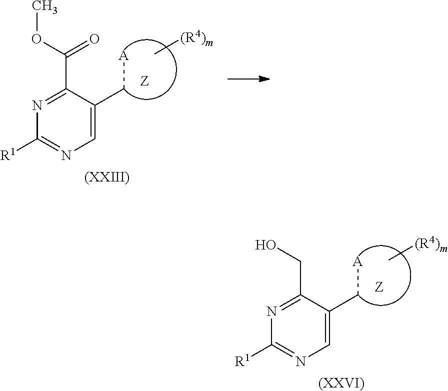

The reduction of pyrimidines (XXIII) is usually carried out from −80° C. to the boiling point of the reaction mixture, preferably from −20° C. to 60° C., particularly preferably from 0° C. to 25° C., in an inert organic solvent.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably diethyl ether, dioxane and THF.

It is also possible to use mixtures of the solvents mentioned.

Examples of reducing agents for pyrimidines (XXIII) include LAH, DIBALH, LiBH₄ or Lithium triethylborohydride.

Preferred agents include LAH and DIBALH.

The hydride-source is used preferably from 1 to 10 equivalents based on the pyrimidine (XXIII), more preferably from 1.0 to 5.0 equivalents based on the pyrimidine (XXIII), most preferably from 1.2 to 2.5 equivalents based on the pyrimidine (XXIII).

Process F:

The pyrimidines of formula (XXVII) can be obtained by reacting respective pyrimidines of formula (XXIII) with a metal organic species like a Grignard reagent (R′MgX, X=Cl, Br, I; R′=alkyl, haloalkyl, alkenyl, alkynyl, cycloalkyl, phenyl, heterocyclyl, or heteroaryl).

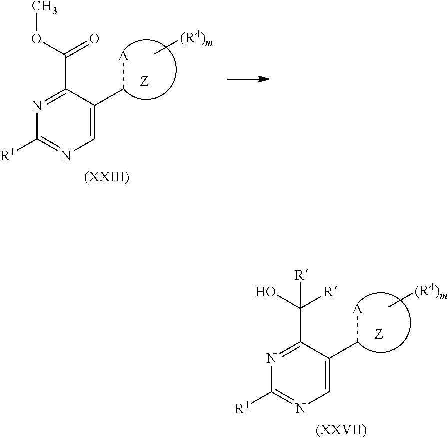

The reaction of pyrimidines (XXIII) with a metal organic species is usually carried out from −80° C. to the boiling point of the reaction mixture, preferably from −20° C. to 60° C., particularly preferably from −20° C. to 25° C., in an inert organic solvent.

Suitable solvents are aliphatic hydrocarbons, such as pentane, hexane, cyclohexane and mixtures of C₅-C₅-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably diethyl ether, dioxane and THF.

It is also possible to use mixtures of the solvents mentioned.

Examples of metal organic species for the synthesis of (XXVII) Grignard reagents like R′MgCl, R′MgBr or R′Mgl, lithium organic species, aluminum organic species like R′₃Al, R′₂AlX and R′AlX₂, titanium organic species like R′₄Ti, R′₃TiX, R′₂TiX₂ and R′TiX₃, Preferred agents include Grignard reagents and lithium organic species.

The metal organic species is used preferably from 2 to 10 equivalents based on the pyrimidine (XXIII), more preferably from 2.0 to 5.0 equivalents based on the pyrimidine (XXIII), most preferably from 2.0 to 3.0 equivalents based on the pyrimidine (XXIII).

Process G:

The pyrimidines of formula (XXVIII) can be obtained by reacting respective pyrimidines of formula (XXIX) with a metal organic species like a Grignard reagent (R′MgX, X=Cl, Br, I; R′=alkyl, haloalkyl, alkenyl, alkynyl, cycloalkyl, phenyl, heterocyclyl or heteroaryl).

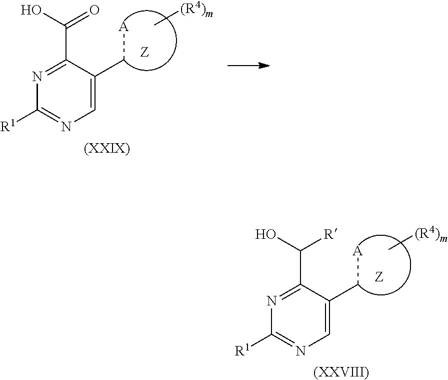

The reaction of pyrimidines (XXIX) with a metal organic species is usually carried out from −80° C. to the boiling point of the reaction mixture, preferably from −20° C. to 60° C., particularly preferably from −20° C. to 25° C. in an inert organic solvent.

Suitable solvents are aliphatic hydrocarbons, such as pentane, hexane, cyclohexane and mixtures of C₅-C₅-alkanes, aromatic hydrocarbons, such as toluene, o-, m- and p-xylene, ethers, such as diethyl ether, diisopropyl ether, tert-butyl methyl ether, dioxane, anisole and tetrahydrofuran, and also dimethyl sulfoxide, DMF and DMAC, particularly preferably diethyl ether, dioxane and tetrahydrofuran.

It is also possible to use mixtures of the solvents mentioned.

Examples of metal organic species for the synthesis of pyrimidines (XXVIII) are Grignard reagents like R′MgCl, R′MgBr or R′Mgl, lithium organic species, aluminum organic species like R′₃Al, R′₂AlX and R′AlX₂, titanium organic species like R′₄Ti, R′₃TiX, R′₂TiX₂ and R′TiX₃, Preferred agents include Grignard reagents and lithium organic species.

The metal organic species is used preferably from 2 to 10 equivalents based on the pyrimidine (XXIX), more preferably from 2.0 to 5.0 equivalents based on the pyrimidine (XXIX), most preferably from 2.0 to 3.0 equivalents based on the pyrimidine (XXIX).

Process H:

The pyrimidines of formula (XXIX) can be obtained by oxidizing respective pyrimidines of formula (XXVI).

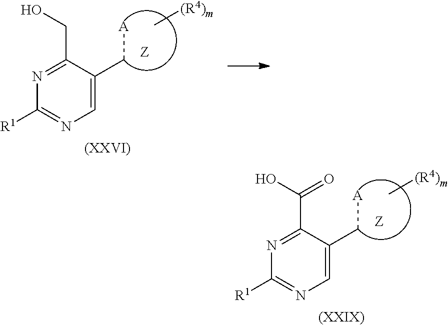

The oxidation of pyrimidines (XXVI) is usually carried out from −80° C. to the boiling point of the reaction mixture, preferably from −20° C. to 100° C., particularly preferably from 0° C. to 75° C., in an inert organic solvent.

The reaction may in principle be carried out in substance. However, preference is given to reacting the pyrimidines (XXVI) in an organic solvent.

Suitable in principle are all solvents which are capable of dissolving the pyrimidines (XXVI) at least partly and preferably fully under the reaction conditions.

Examples of suitable solvents are aromatic hydrocarbons such as benzene, chlorobenzene, toluene, cresols, o-, m- and p-xylene, halogenated hydrocarbons such as CH₂Cl₂, CHCl₃, CCH₂ClCH₂Cl or CCl₄, ethers such as diethyl ether, diisopropyl ether, TBME, dioxane, anisole and THF, as well as dipolar aprotic solvents such as sulfolane, dimethylsulfoxide, DMF, N,N-DMAC (DMAC), DMIDMI, DMPU, DMSO and NMP.

Preferred solvents are halogenated hydrocarbons such as CH₂Cl₂, CHCl₃, CCH₂ClCH₂C₁ or CCl₄,and dipolar aprotic solvents such as sulfolane, dimethylsulfoxide, DMF, DMAC, DMIDMI, N,N′-dimethyl¬propylene urea (DMPU), DMSO and NMP.

More preferred solvents halogenated hydrocarbons such as CH₂Cl₂, CHCl₃, CCH₂ClCH₂C₁ or CCl₄.

It is also possible to use mixtures of the solvents mentioned.

Examples of oxidizing agents for the synthesis of pyrimidines (XXIX) are metal oxides such as MnO₂, KMnO₄, CrO₃ or PCC, and non-metal oxides such as NaClO, NalO₄ or pyridine/SO₃-complex. In addition methods like the Swern oxidation or the TEMPO oxidation known to a person skilled in the art can be used to obtain pyridines of formula (XXIX).

Preferred agents include MnO₂, KMnO₄ and PCC, more preferred is MnO₂.

The oxidizing agent is used preferably from 1 to 50 equivalents based on the pyrimidine (XXVI), more preferably from 1.0 to 20.0 equivalents based on the pyrimidine (XXVI), most preferably from 1.0 to 10.0 equivalents based on the pyrimidine (XXVI).

Process I:

The pyrimidines of formula (XXX; Q=cycloalkyl, halocycloalkyl, alkyl, haloalkyl, alkenyl, alkynyl, phenyl, heteroaryl, heterocyclyl, alkylidenyl or halo alkylidenyl and R′=alkyl, haloalkyl, alkenyl, alkynyl, cycloalkyl, heterocyclyl, alkoxycarbonylalkyl) can be obtained by reacting respective pyrimidines of formula (XXXI) with base and an electrophile.

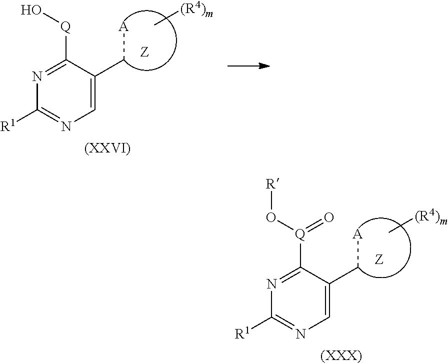

Electrophiles can be an alkyl-, alkenyl- or alkynyl-halide, e.g. methyl iodide, allyl bromide propargyl bromide, ethyl iodide, propyl bromide, or ethyl 2-bromoacetate.

The reaction of the pyrimidine (XXXI) with the electrophile is usually carried out at temperatures of from −100° C. to the boiling point of the reaction mixture, preferably from −20° C. to 100° C., particularly preferably from −0° C. to 30° C., in an inert organic solvent in the presence of a base.

Suitable in principle are all solvents which are capable of dissolving the pyrimidine (XXXI) and the electrophile at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, nitriles such as acetonitrile and propionitrile, as well as dipolar aprotic solvents such as sulfolane, dimethylsulfoxide, DMF, N,N-DMAC (DMAC), DMIDMI, DMPU, DMSO and NMP.

Preferred solvents are ethers such as diethyl ether, diisopropyl ether, TBME, dioxane, anisole and THE and dipolar aprotic solvents such as sulfolane, dimethylsulfoxide, DMF, N,N-DMAC (DMAC), DMIDMI, DMPU, DMSO and NMP.

More preferred solvents are dipolar aprotic solvents such as dimethylsulfoxide, DMF, and NMP.

It is also possible to use mixtures of the solvents mentioned.

Suitable bases are, in general, inorganic compounds, such as alkali metal and alkaline earth metal anhydrides, such as lithium hydride, sodium hydride, potassium hydride and calcium hydride, alkali metal azides, such as lithium hexamethyidisilazide, organometallic compounds, in particular alkali metal alkyls, such as methyllithium, butyllithium and phenyllithium, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, e.g. tertiary amines, such as trimethylamine, triethylamine, diisopropylethylamine and N-methylpiperidine, pyridine, substituted pyridines, such as collidine, lutidine and 4-dimethylaminpyridine, and also bicyclic amines. Particular preference is given to sodium hydride, lithium hexamethyldisilazide and lithium diisopropylamide.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile, based on the pyrimidine (XXXI).

Process J:

The pyrimidine compounds of formula (I) can in addition be obtained by reacting respective pyrimidine boronic acid esters of formula (XXXII) with halides of formula (XXXIII) in which X equals Cl, Br, or I:

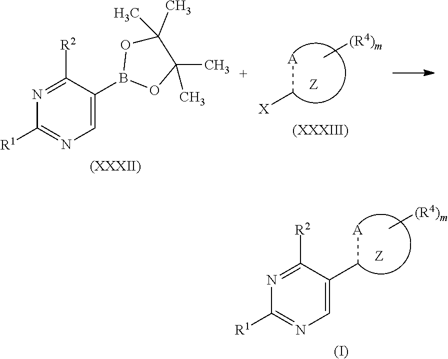

The reaction of the pyrimidine (XXXII) with halides of formula (XXXIII) is usually carried out from 0° C. to the boiling point of the reaction mixture, preferably from 15° C. to 110° C., particularly preferably from 40° C. to 100° C., in an inert organic solvent in the presence of a base and a catalyst.

The reaction may in principle be carried out in substance. However, preference is given to reacting the pyrimidines (XXXII) with halides of formula (XXXIII) in an organic solvent with or without water as co-solvent.

Suitable in principle are all solvents which are capable of dissolving the pyrimidines (XXXII) with halides of formula (XXXIII) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, N,N-DMAC (DMAC), DMIDMI, DMPU, DMSO and NMP.

Preferred solvents are ethers such as diethyl ether, diisopropyl ether, TBME, dioxane, anisole and THF and dipolar aprotic solvents such as sulfolane, dimethylsulfoxide, DMF, N,N-DMAC (DMAC), DMIDMI, N,N′-dimethyl¬propylene urea (DMPU), DMSO and NMP.

More preferred solvents are ethers such as diethyl ether, diisopropyl ether, TBME, dioxane, anisole and THF.

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂).

The term base as used herein also includes mixtures of two or more, preferably two of the above bases. Particular preference is given to the use of one base.

The bases are used preferably from 1 to 10 equivalents based on the pyrimidine (XXXII), more preferably from 1.0 to 5.0 equivalents based on the pyrimidine (XXXII), most preferably from 1.2 to 2.5 equivalents based on the pyrimidine (XXXII).

It may be advantageous to add the base offset over a period of time.

The reaction of the pyrimidines (XXXII) with halides of formula (XXXIII) is carried out in the presence of a catalyst. Examples of suitable catalysts include e.g., palladium based catalysts like, e.g., Palladium(II)acetate, tetrakis(triphenylphosphine)palladium(0), bis(triphenylphosphine) palladium(II)chloride or (1,1,-bis(diphenylphosphino)ferrocene)-dichloropalladium (II), and optionally suitable additives such as, e.g., phosphines like, e.g., P(o-tolyl)₃, triphenylphosphine or BINAP (2,2′-Bis(diphenylphospino)-1,1′-binaphthyl).

The amount of catalyst is usually 0.01 to 20 mol % (0.0001 to 0.2 equivalents) based on the pyrimidine (XXXII).

The halides (XXXIII) required for the preparation of pyrimidine compounds of formula (I) are known from the literature or are commercially available.

The pyrimidines of formula (XXXII) can be obtained by reacting the respective pyrimidines (XXXIV) with Bis(pinacolato)diboron XX)V.

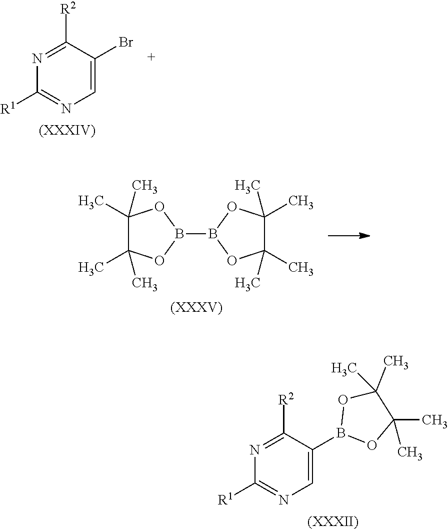

The reaction of the pyrimidine (XXXIV) with Bis(pinacolato)diboron XXXV is usually carried out from 0° C. to the boiling point of the reaction mixture, preferably from 15° C. to 110° C., particularly preferably from 40° C. to 100° C., in an inert organic solvent in the presence of a base and a catalyst.

The reaction may in principle be carried out in substance. However, preference is given to reacting the pyrimidines (XXXIV) with Bis(pinacolato)diboron XXXV in an organic solvent with or without water as co-solvent.

Suitable in principle are all solvents which are capable of dissolving the pyrimidines (XXXIV) with Bis(pinacolato)diboron XXXV at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, DMAC, DMIDMI, DMPU, DMSO and NMP.

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂); alkali metal and alkaline earth metal acetates such as sodium acetate or potassium acetate.

The term base as used herein also includes mixtures of two or more, preferably two of the above bases. Particular preference is given to the use of one base.

The bases are used preferably from 1 to 10 equivalents based on the pyrimidine (XXXIV), more preferably from 1.0 to 5.0 equivalents based on the pyrimidine (XXXIV), most preferably from 1.2 to 2.5 equivalents based on the pyrimidine (XXXIV).

It may be advantageous to add the base offset over a period of time.

The reaction of the pyrimidines (XXXIV) with Bis(pinacolato)diboron XXXV is carried out in the presence of a catalyst. Examples of suitable catalysts include e.g., palladium based catalysts like, e.g., Palladium(II)acetate, tetrakis(triphenylphosphine)palladium(0), bis(triphenylphosphine) palladium(II)chloride or (1,1,-bis(diphenylphosphino)ferrocene)-dichloropalladium (II), and optionally suitable additives such as, e.g., phosphines like, e.g., P(o-tolyl)₃, triphenylphosphine or BINAP (2,2′-Bis(diphenylphospino)-1,1′-binaphthyl).

The amount of catalyst is usually 0.01 to 20 mol % (0.0001 to 0.2 equivalents) based on the pyrimidine (XXXIV).

The halides (XXXIV) required for the preparation of pyrimidine compounds of formula (XXXII) are known from the literature or are commercially available.

The end of the reactions can easily be determined by the skilled worker by means of routine methods.

The reaction mixtures are worked up in a customary manner, e.g. by mixing with water, separation of the phases and, if appropriate, chromatographic purification of the crude product.

Some of the intermediates and end products are obtained in the form of viscous oils, which can be purified or freed from volatile components under reduced pressure and at moderately elevated temperature.

If the intermediates and the end products are obtained as solid, purification can also be carried out by recrystallization or digestion.

The present invention also provides agrochemical compositions comprising at least one pyrimidine compounds of formula (I) and auxiliaries customary for formulating crop protection agents.

The present invention furthermore provides a method for controlling unwanted vegetation where a herbicidal effective amount of at least one pyrimidine compounds of formula (I) is allowed to act on plants, their seeds and/or their habitat. Application can be done before, during and/or after, preferably during and/or after, the emergence of the undesirable plants.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

As used herein, the terms “controlling” and “combating” are synonyms.

As used herein, the terms “undesirable vegetation” and “harmful plants” are synonyms.

If the pyrimidine compounds of formula (I) as described herein are capable of forming geometrical isomers, e.g. E/Z isomers, it is possible to use both, the pure isomers and mixtures thereof, in the compositions according to the invention.

If the pyrimidine compounds of formula (I) as described herein have one or more centres of chirality and, as a consequence, are present as enantiomers or diastereomers, it is possible to use both, the pure enantiomers and diastereomers and their mixtures, in the compositions according to the invention.

If the pyrimidine compounds of formula (I) as described herein have ionisable functional groups, preferably an acidic functionality, more preferably a carboxylic group or a sulphonic group, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four H atoms are replaced by C₁-C₄-alkyl, HO—C₁-C₄-alkyl, C₁-C₄-alkoxy-C₁-C₄-alkyl, HO—C₁-C₄-alkoxy-C₁-C₄-alkyl, phenyl or benzyl, preferably ammonium, methylammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-HO-ethyl)ammonium (trolamine salt), tris(2-hydroxypropyl)ammonium, benzyltrimethylammonium, benzyltriethylammonium, N,N,N-trimethylethanolammonium (choline salt), furthermore phosphon-ium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogen-sulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

Pyrimidine compounds of formula (I) as described herein having an acidic functionality, preferably a carboxylic group or a sulphonic group, can be employed, if applicable, in the form of the acid, in the form of an agriculturally suitable salt with the cations as defined above or else in the form of an agriculturally acceptable derivative, e.g. as amides, such as mono- and di-C₁-C₆-alkylamides or arylamides, as esters, e.g. as allyl esters, propargyl esters, C₁-C₁₀-alkyl esters, alkoxyalkyl esters, tefuryl ((tetrahydrofuran-2-yl)methyl) esters and also as thioesters, e.g. as C₁-C₁₀-alkylthio esters. Preferred mono- and di-C₁-C₆-alkylamides are the CH₃ and the dimethylamides. Preferred arylamides are, e.g., the anilides and the 2-chloroanilides. Preferred alkyl esters are, e.g., the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, e.g. the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

The organic moieties mentioned in the definition of the variables R¹, R², A, Z, R³, R^{3A}, and R⁴, are—like the term halogen—collective terms for individual enumerations of the individual group members. The term halogen denotes in each case F, Cl, Br, or I. All hydrocarbon chains, e.g. all alkyl, alkenyl, alkynyl, alkoxy chains can be straight-chain or branched, the prefix COₙ—Cₘ denoting in each case the possible number of carbon atoms in the group.

Examples of such meanings are:
C₁-C₄-alkyl: e.g. CH₃, C₂H₅, n-propyl, CH(CH₃)₂, n-butyl, CH(CH₃)—C₂H₅, CH₂—CH(CH₃)₂, and C(CH₃)₃;
C₁-C₆-alkyl: C₁-C₄-alkyl as mentioned above, and also, e.g., n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl, or n-hexyl;
C₁-C₄-haloalkyl: C₁-C₄-alkyl as mentioned above which is partially or fully substituted by fluorine, chlorine, bromine and/or iodine, e.g., chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoro-propyl, 1-(fluoromethyl)-2-fluoroethyl, 1-(chloromethyl)-2-chloroethyl, 1-(bromomethyl)-2-bromo-ethyl, 4-fluorobutyl, 4-chlorobutyl, 4-bromobutyl, nonafluorobutyl, 1,1,2,2,-tetrafluoroethyl, and 1-trifluoromethyl-1,2,2,2-tetrafluoroethyl;
C₁-C₆-haloalkyl: C₁-C₄-haloalkyl as mentioned above, and also, e.g., 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl, and dodecafluorohexyl;
C₃-C₆-cycloalkyl: monocyclic saturated hydrocarbons having 3 to 6 ring members, such as cyclopropyl, cyclobutyl, cyclopentyl, and cyclohexyl;
C₃-C₆-alkenyl: e.g. 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl;
C₃-C₆-haloalkenyl: a C₃-C₆-alkenyl substituent as mentioned above which is partially or fully substituted by fluorine, chlorine, bromine and/or iodine, e.g. 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl, or 2,3-dibromobut-2-en-1-yl;
C₃-C₆-alkynyl: e.g. 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl;
C₂-C₆-alkynyl: C₃-C₆-alkynyl as mentioned above and also ethynyl;
C₃-C₆-haloalkynyl: a C₃-C₆-alkynyl group as mentioned above which is partially or fully substituted by F, Cl, Br and/or I, e.g. 1,1-difluoroprop-2-yn-1-yl, 3-chloroprop-2-yn-1-yl, 3-bromo-prop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl, or 6-iodohex-5-yn-1-yl;
C₁-C₄-alkoxy: e.g. methoxy, ethoxy, propoxy, 1-methylethoxy butoxy, 1-methylpropoxy, 2-methylpropoxy, and 1,1-dimethylethoxy;
C₁-C₆-alkoxy: C₁-C₄-alkoxy as mentioned above, and also, e.g., pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy.
C₁-C₄-haloalkoxy: a C₁-C₄-alkoxy group as mentioned above which is partially or fully substituted by fluorine, chlorine, bromine and/or iodine, i.e., e.g., fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, and nonafluorobutoxy;
C₁-C₆-haloalkoxy: a C₁-C₄-haloalkoxy as mentioned above, and also, e.g., 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and dodecafluorohexoxy;
C₁-C₄-alkylthio: e.g. methylthio, ethylthio, propylthio, 1-methylethylthio, butylthio, 1-methylpropylthio, 2-methylpropylthio, and 1,1-dimethylethylthio;
C₁-C₆-alkylthio: C₁-C₄-alkylthio as mentioned above, and also, e.g., pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio, and 1-ethyl-2-methylpropylthio;
(C₁-C₄-alkyl)amino: e.g. methylamino, ethylamino, propylamino, 1-methylethylamino, butylamino, 1-methylpropylamino, 2-methylpropylamino, or 1,1-dimethylethylamino;
(C₁-C₆-alkyl)amino: (C₁-C₄-alkylamino) as mentioned above, and also, e.g., pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutylamino 3,3-dimethylbutylamino, 1-ethylbutylamino, 2-ethylbutylamino, 1,1,2-trimethylpropyl-amino, 1,2,2-trimethyl-propylamino, 1-ethyl-1-methylpropylamino, or 1-ethyl-2-methylpropylamino;
di(C₁-C₄-alkyl)amino: e.g. N,N-dimethylamino, N,N-diethylamino, N,N-di(1-meth-ylethyl)amino, N,N-dipropylamino, N,N-dibutylamino, N,N-di(1-methylpropyl)amino, N,N-di(2-methylpropyl)amino, N,N-di(1,1-dimethylethyl)amino, N-ethyl-N-methylamino, N-methyl-N-propylamino, N-methyl-N-(1-methylethyl)amino, N-butyl-N-methylamino, N-methyl-N-(1-methylpropyl)-amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)-amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl) amino, N-(1-methylethyl)-N-(1-methylpropyl)amino, N-(1-methylethyl)-N-(2-methylpropyl)-amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino, or N-(1,1-dimethylethyl)-N-(2-methylpropyl)amino;
di(C₁-C₆-alkyl)amino: di(C₁-C₄-alkyl)amino as mentioned above, and also, e.g., N-methyl-N-pentylamino, N-methyl-N-(1-methylbutyl)amino, N-methyl-N-(2-methylbutyl)amino, N-methyl-N-(3-methylbutyl)amino, N-methyl-N-(2,2-dimethylpropyl)amino, N-methyl-N-(1-ethylpropyl) amino, N-methyl-N-hexylamino, N-methyl-N-(1,1-dimethylpropyl)amino, N-methyl-N-(1,2-di-methylpropyl)amino, N-methyl-N-(1-methylpentyl)amino, N-methyl-N-(2-methylpentyl)amino, N-methyl-N-(3-methylpentyl)amino, N-methyl-N-(4-methylpentyl)amino, N-methyl-N-(1,1-dimethylbutyl) amino, N-methyl-N-(1,2-dimethylbutyl)amino, N-methyl-N-(1,3-dimethylbutyl)amino, N-methyl-N-(2,2-dimethylbutyl)amino, N-methyl-N-(2,3-dimethylbutyl)amino, N-methyl-N-(3,3-dimethylbutyl) amino, N-methyl-N-(1-ethylbutyl)amino, N-methyl-N-(2-ethylbutyl)amino, N-methyl-N-(1,1,2-trimethylpropyl)amino, N-methyl-N-(1,2,2-trimethylpropyl)amino, N-methyl-N-(1-ethyl-1-methylpropyl)amino, N-methyl-N-(1-ethyl-2-methylpropyl)amino, N-ethyl-N-pentylamino, N-ethyl-N-(1-methylbutyl)amino, N-ethyl-N-(2-methylbutyl)amino, N-ethyl-N-(3-methylbutyl)amino, N-ethyl-N-(2,2-dimethylpropyl)amino, N-ethyl-N-(1-ethylpropyl)amino, N-ethyl-N-hexylamino, N-ethyl-N-(1,1-dimethylpropyl)amino, N-ethyl-N-(1,2-dimethylpropyl)amino, N-ethyl-N-(1-methylpentyl)amino, N-ethyl-N-(2-methylpentyl)amino, N-ethyl-N-(3-methylpentyl)amino, N-ethyl-N-(4-methylpentyl)amino, N-ethyl-N-(1,1-dimethylbutyl)amino, N-ethyl-N-(1,2-dimethyl-butyl)amino, N-ethyl-N-(1,3-dimethylbutyl)amino, N-ethyl-N-(2,2-dimethylbutyl)amino, N-ethyl-N-(2,3-dimethylbutyl)amino, N-ethyl-N-(3,3-dimethylbutyl)amino, N-ethyl-N-(1-ethylbutyl)amino, N-ethyl-N-(2-ethylbutyl)amino, N-ethyl-N-(1,1,2-trimethylpropyl)amino, N-ethyl-N-(1,2,2-trimethylpropyl) amino, N-ethyl-N-(1-ethyl-1-methylpropyl)amino, N-ethyl-N-(1-ethyl-2-methylpropyl)-amino, N-propyl-N-pentylamino, N-butyl-N-pentylamino, N,N-dipentylamino, N-propyl-N-hexylamino, N-butyl-N-hexylamino, N-pentyl-N-hexylamino, or N,N-dihexylamino;
C₁-C₆-alkylsulfinyl (C₁-C₆-Alkyl-S(═O)—): e.g. methylsulfinyl, ethylsulfinyl, propylsulfinyl, 1-methylethylsulfinyl, butylsulfinyl, 1-methylpropylsulfinyl, 2-methylpropylsulfinyl, 1,1-di-methylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropyl-sulfinyl, hexylsulfinyl, 1-methylpentylsulfinyl, 2-methylpentylsulfinyl, 3-methylpentylsulfinyl, 4-methylpentyl-sulfinyl, 1,1-dimethylbutylsulfinyl, 1,2-dimethylbutylsulfinyl, 1,3-dimethylbutyl-sulfinyl, 2,2-dimethylbutylsulfinyl, 2,3-dimethylbutylsulfinyl, 3,3-dimethylbutyl-sulfinyl, 1-ethylbutyl-sulfinyl, 2-ethylbutylsulfinyl, 1,1,2-trimethylpropylsulfinyl, 1,2,2-trimethylpropylsulfinyl, 1-ethyl-1-methylpropyl-sulfinyl, and 1-ethyl-2-methylpropylsulfinyl;
C₁-C₆-alkylsulfonyl (C₁-C₆-alkyl-S(O)₂—): e.g. methylsulfonyl, ethylsulfonyl, propylsulfonyl, 1-methylethylsulfonyl, butylsulfonyl, 1-methylpropylsulfonyl, 2-methyl-propylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropyl-sulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutyl-sulfonyl, 1-ethylbutylsulfonyl, 2-ethylbutylsulfonyl, 1,1,2-trimethyl-propylsulfonyl, 1,2,2-trimethylpropylsulfonyl, 1-ethyl-1-methylpropylsulfonyl, and 1-ethyl-2-methylpropylsulfonyl;
C₃-C₆-cycloalkyl: a monocyclic saturated hydrocarbon having 3 to 6 ring members, such as cyclopropyl, cyclobutyl, cyclopentyl, and cyclohexyl;
C₃-C₆-cycloalkenyl: 1-cyclopropenyl, 2-cyclopropenyl, 1-cyclobutenyl, 2-cyclobutenyl, 1-cyclopentenyl, 2-cyclopentenyl, 1,3-cyclopentadienyl, 1,4-cyclopentadienyl, 2,4-cyclopentadienyl, 1-cyclohexenyl, 2-cyclohexenyl, 3-cyclohexenyl, 1,3-cyclohexadienyl, 1,4-cyclohexadienyl, or 2,5-cyclohexadienyl;
heterocyclyl: a 3- to 6-membered heterocyclyl: a saturated or partial unsaturated cycle having three to six ring members which comprises apart from carbon atoms one to four nitrogen atoms, or one or two oxygen atoms, or one or two sulfur atoms, or one to three nitrogen atoms and an oxygen atom, or one to three nitrogen atoms and a sulfur atom, or one sulfur and one oxygen atom, e.g.

3- or 4-membered heterocycles like 2-oxiranyl, 2-aziridinyl, 2-thiiranyl, 2-oxetanyl, 3-oxetanyl, 2-thietanyl, 3-thietanyl, 1-azetidinyl, 2-azetidinyl, 1-azetinyl, or 2-azetinyl;

5-membered saturated heterocycles like2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 2-isothiazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 3-oxazolidinyl, 1,2,4-oxadiazolidin-3-yl, 1,2,4-oxadiazolidin-5-yl, 3-thiazolidinyl, 1,2,4-thiadiazolidin-3-yl, 1,2,4-thiadiazolidin-5-yl, 1,2,4-triazolidin-3-yl, 1,2,4-oxadiazolidin-2-yl, 1,2,4-oxadiazolidin-4-yl, 1,3,4-oxadiazolidin-2-yl, 1,2,4-thiadiazolidin-2-yl, 1,2,4-thiadiazolidin-4-yl, 1,3,4-thiadiazolidin-2-yl, 1,2,4-triazolidin-1-yl, or 1,3,4-triazolidin-2-yl; 5-membered partial unsaturated heterocycles like 2,3-dihydrofur-2-yl, 2,3-dihydrofur-3-yl, 2,4-dihydrofur-2-yl, 2,4-dihydrofur-3-yl, dioxolan-2-yl, 1,3-dioxol-2-yl, 2,3-dihydrothien-2-yl, 2,3-dihydrothien-3-yl, 2,4-dihydrothien-2-yl, 2,4-dihydrothien-3-yl, 4,5-dihydropyrrol-1-yl, 4,5-dihydropyrrol-2-yl, 4,5-dihydropyrrol-3-yl, 2,5-dihydropyrrol-1-yl, 2,5-dihydropyrrol-2-yl, 2,5-dihydropyrrol-3-yl, 2,3-dihydroisoxazol-1-yl, 2,3-dihydroisoxazol-3-yl, 2,3-dihydroisoxazol-4-yl, 2,3-dihydroisoxazol-5-yl, 2,5-dihydroisoxazol-3-yl, 2,5-dihydroisoxazol-4-yl, 2,5-dihydroisoxazol-5-yl, 4,5-dihydroisoxazol-2-yl, 4,5-dihydroisoxazol-3-yl, 4,5-dihydroisoxazol-4-yl, 4,5-dihydroisoxazol 5-yl, 2,3-dihydroisothiazol-1-yl, 2,3-dihydroisothiazol-3-yl, 2,3-dihydroisothiazol-4-yl, 2,3-dihydroisothiazol-5-yl, 2,5-dihydroisothiazol-3-yl, 2,5-dihydroisothiazol-4-yl, 2,5-dihydroisothiazol-5-yl, 4,5-dihydroisothiazol-1-yl, 4,5-dihydroisothiazol-3-yl, 4,5-dihydroisothiazol-4-yl, 4,5-dihydroisothiazol-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydroimidazol-1-yl, 2,3-dihydroimidazol-2-yl, 2,3-dihydroimidazol-3-yl, 2,3-dihydroimidazol-4-yl, 2,3-dihydroimidazol-5-yl, 4,5-dihydroimidazol-1-yl, 4,5-dihydroimidazol-2-yl, 4,5-dihydroimidazol-4-yl, 4,5-dihydroimidazol-5-yl, 2,5-dihydroimidazol-1-yl, 2,5-dihydroimidazol-2-yl, 2,5-dihydroimidazol-4-yl, 2,5-dihydroimidazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 2,3-dihydrothiazol-2-yl, 2,3-dihydrothiazol-3-yl, 2,3-dihydrothiazol-4-yl, 2,3-dihydrothiazol-5-yl, 3,4-dihydrothiazol-2-yl, 3,4-dihydrothiazol-3-yl, 3,4-dihydrothiazol-4-yl, 3,4-dihydrothiazol-5-yl, 3,4-dihydrothiazol-2-yl, 3,4-dihydrothiazol-3-yl, or 3,4-dihydrothiazol-4-yl;

6-membered saturated heterocycles like 1-piperidinyl, 2-piperidinyl, 3-piperidinyl, 4-piperidinyl, 1,3-dioxan-5-yl, 1,4-dioxanyl, 1,3-dithian-5-yl, 1,3-dithianyl, 1,3-oxathian-5-yl, 1,4-oxathianyl, 2-tetrahydropyranyl, 3-tetrahydopyranyl, 4-tetrahydropyranyl, 2-tetrahydrothiopyranyl, 3-tetrahydrothiopyranyl, 4-tetrahydrothiopyranyl, 1-hexahydropyridazinyl, 3-hexahydropyridazinyl, 4-hexahydropyridazinyl, 1-hexahydropyrimidinyl, 2-hexahydropyrimidinyl, 4-hexahydropyrimidinyl, 5-hexahydropyrimidinyl, 1-piperazinyl, 2-piperazinyl, 1,3,5-hexahydrotriazin-1-yl, 1,3,5-hexahydrotriazin-2-yl, 1,2,4-hexahydrotriazin-1-yl, 1,2,4-hexahydrotriazin-3-yl, tetrahydro-1,3-oxazin-1-yl, tetrahydro-1,3-oxazin-2-yl, tetrahydro-1,3-oxazin-6-yl, 1-morpholinyl, or 2-morpholinyl, 3-morpholinyl;

6-membered partial unsaturated heterocycles like 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, or 5,6-dihydro-4H-1,3-oxazin-2-yl.
heteroaryl: a 5- or 6-membered heteroaryl: monocyclic aromatic heteroaryl having 5 to 6 ring members which, in addition to carbon atoms and independent of their position in the ring, contains 1 to 4 nitrogen atoms, or 1 to 3 nitrogen atoms and an oxygen or sulfur atom, or an oxygen or a sulfur atom, e.g. 5-membered aromatic rings like furyl (e.g. 2-furyl, 3-furyl), thienyl (e.g. 2-thienyl, 3-thienyl), pyrrolyl (e.g. pyrrol-2-yl, pyrrol-3-yl), pyrazolyl (e.g. pyrazol-3-yl, pyrazol-4-yl), isoxazolyl (e.g. isoxazol-3-yl, isoxazol-4-yl, isoxazol-5-yl), isothiazolyl (e.g. isothiazol-3-yl, isothiazol-4-yl, isothiazol-5-yl), imidazolyl (e.g. imidazole-2-yl, imidazole-4-yl), oxazolyl (e.g. oxazol-2-yl, oxazol-4-yl, oxazol-5-yl), thiazolyl (e.g. thiazol-2-yl, thiazol-4-yl, thiazol-5-yl), oxadiazolyl (e.g. 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl), thiadiazolyl (e.g. 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl), triazolyl (e.g. 1,2,3-triazol-4-yl, 1,2,4-triazol-3-yl); 1-tetrazolyl; 6-membered aromatic rings like pyridyl (e.g. pyridine-2-yl, pyridine-3-yl, pyridine-4-yl), pyrazinyl (e.g. pyridazin-3-yl, pyridazin-4-yl), pyrimidinyl (e.g. pyrimidin-2-yl, pyrimidin-4-yl, pyrimidin-5-yl), pyrazin-2-yl, triazinyl (e.g. 1,3,5-triazin-2-yl, or 1,2,4-triazin-3-yl, 1,2,4-triazin-5-yl, 1,2,4-triazin-6-yl).

The term “substituted” if not specified otherwise refers to substituted by 1, 2 or maximum possible number of substituents. If substituents as defined in compounds of formula I are more than one then they are independently from each other are same or different if not mentioned otherwise.

The term “acidic functionality” if not specified otherwise refers to a functionality capable of donating a hydrogen (proton or hydrogen ion H⁺), such as a carboxylic group or a sulphonic group, or, alternatively, capable of forming a covalent bond with an electron pair.

The terms “compounds of formula (I)”, “Pyrimidine compounds of formula (I)”, “Compounds I” and “compounds of invention” are synonyms.

The term “cyclic groups” comprises aliphatic cyclic groups such as cycloalkyl, cycloalkenyl and heterocyclyl and aromatic cyclic groups such as heteroaryl and phenyl.

The preferred embodiments of the invention mentioned herein below have to be understood as being preferred either independently from each other or in combination with one another.

In general, pyrimidine compounds of formula (I) are suitable as herbicides.

According to a preferred embodiment of the invention preference is given pyrimidine compounds of formula (I), and their use as herbicides, wherein the variables, either independently of one another or in combination with one another, have the following meanings:

Preferred R¹ is C₁-C₆-alkyl, C₁-C₆-alkoxy, C₁-C₆-haloalkoxy, C₃-C₆-alkenyloxy, C₃-C₆-haloalkenyloxy C₃-C₆-alkynyloxy, C₄-C₆-haloalkynyloxy, C₁-C₆-alkylthio, C₃-C₆-cycloalkyl, wherein the cycloalkyl substituent is unsubstituted;

particularly preferred R¹ is C₁-C₆-alkyl, C₁-C₆-alkoxy, or C₃-C₆-cycloalkyl, wherein the cycloalkyl substituent is unsubstituted;

especially preferred R¹ is C₃-C₆-cycloalkyl, wherein the cycloalkyl substituent is unsubstituted;

also especially preferred R¹ is C₂H₅, i-C₃H₇, i-C₄H₅, OCH₃, c-C₃H₅, or c-C₄H₉;

more preferred R¹ is C₂H₅, OCH₃, or c-C₃H₅;

most preferred R¹ is c-C₃H₅.

Preferred R² is C₂-C₆-alkenyl, C₂-C₆-haloalkenyl, C₁-C₆-alkoxy-C₂-C₆-alkenyl, C₂-C₆-alkynyl, C₃-C₆-cycloalkenyl, C₃-C₆-cycloalkenyl-C₁-C₆-alkyl, C₃-C₆-cycloalkyl-C₁-C₆-alkylidenyl, C₃-C₆-halocycloalkyl-C₁-C₆-alkylidenyl, C₃-C₆-cycloalkenyl-C₁-C₆-alkylidenyl, C₃-C₆-hydroxycycloalkyl-C₁-C₆-alkyl, C₃-C₆-hydroxycycloalkenyl-C₁-C₆-alkyl, C₁-C₆-hydroxyalkyl, C₃-C₆-cycloalkyl-C₂-C₆-hydroxyalkylidenyl, hydroxycarbonyl-C₁-C₆-alkyl, hydroxycarbonyl-C₁-C₆-haloalkyl, C₁-C₆-alkoxycarbonyl-C₁-C₆-alkyl, C₃-C₆-hydroxycycloalkyl-C₁-C₆-hydroxyalkyl, C₂-C₆-dihydroxyalkyl, C₃-C₆-cycloalkyl-C₃-C₆-dihydroxyalkylidenyl, hydroxycarbonyl-C₂-C₆-dihydroxyalkyl, C₁-C₆-alkoxycarbonyl-C₂-C₆-dihydroxyalkyl, C₁-C₆-dicyanoalkyl, or 5- or 6-membered heteroaryl;

wherein hydroxy groups of R² are unsubstituted or substituted by R^{b},

cyclic groups of R² are unsubstituted or substituted by R^{c}, and

and acyclic aliphatic groups of R² are unsubstituted or substituted by R^{d}.

particularly preferred R² is C₂-C₆-alkenyl, C₂-C₆-alkynyl, C₃-C₆-cycloalkenyl, C₃-C₆-cycloalkyl-C₁-C₆-alkylidenyl, C₃-C₆-halocycloalkyl-C₁-C₆-alkylidenyl, C₃-C₆-hydroxycycloalkyl-C₁-C₆-alkyl, C₃-C₆-hydroxycycloalkyl-C₁-C₆-hydroxyalkyl, C₂-C₆-dihydroxyalkyl, or 5- or 6-membered heteroaryl;

also particularly preferred R² is C₂-C₆-alkenyl, C₂-C₆-alkynyl, C₃-C₆-cycloalkenyl-C₁-C₆-alkyl, C₃-C₆-cycloalkyl-C₁-C₆-alkylidenyl, C₁-C₆-hydroxyalkyl, C₁-C₆-alkoxycarbonyl-C₁-C₆-alkyl, C₂-C₆-dihydroxyalkyl, C₁-C₆-dicyanoalkyl, 5- or 6-membered heteroaryl;

wherein hydroxy groups of R² are unsubstituted or substituted by R^{b},

cyclic groups of R² are unsubstituted or substituted by R^{c}, and

acyclic aliphatic groups of R² are unsubstituted or substituted by R^{d}.

especially preferred R² is R² is C₂-C₆-alkenyl, C₂-C₆-alkynyl, C₃-C₆-cycloalkyl-C₁-C₆-alkylidenyl, C₃-C₆-hydroxycycloalkyl-C₁-C₆-alkyl, and 5- or 6-membered heteroaryl;

also especially preferred R² is C₂-C₆-alkenyl, C₁-C₆-hydroxyalkyl, C₃-C₆-cycloalkyl-C₁-C₆-alkylidenyl, C₂-C₆-dihydroxyalkyl, C₁-C₆-dicyanoalkyl and 5- or 6-membered heteroaryl;

also especially preferred R² is C₂-C₆-alkenyl, C₃-C₆-cycloalkyl-C₁-C₆-alkylidenyl, C₁-C₆-hydroxyalkyl, C₂-C₆-dihydroxyalkyl, or 5- or 6-membered heteroaryl;

wherein hydroxy groups of R² are unsubstituted or substituted by R^{b},

cyclic groups of R² are unsubstituted or substituted by R^{c}, and

and acyclic aliphatic groups of R² are unsubstituted or substituted by R^{d}.

more preferred R² is C₂-C₆-alkenyl, C₃-C₆-cycloalkyl-C₁-C₆-alkylidenyl, or 5- or 6-membered heteroaryl;

also more preferred R² is C₁-C₆-hydroxyalkyl, C₂-C₆-dihydroxyalkyl, C₃-C₆-cycloalkyl-C₁-C₆-alkylidenyl, or 5- or 6-membered heteroaryl;

wherein OH groups of R² are unsubstituted or substituted by R^{b},

cyclic groups of R² are unsubstituted or substituted by R^{c}, and

acyclic aliphatic groups of R² are unsubstituted or substituted by R^{d}.

most preferred R² is C₂-C₆-alkenyl;

also most preferred R² is C₃-C₆-cycloalkyl-C₁-C₆-alkylidenyl;

also most preferred R² is 5- or 6-membered heteroaryl;

also most preferred R² is 5-membered heteroaryl;

also most preferred R² is C₁-C₆-hydroxyalkyl;

also most preferred R² is C₂-C₆-dihydroxyalkyl;

wherein OH groups of R² are unsubstituted or substituted by R^{b},

cyclic groups of R² are unsubstituted or substituted by R^{c}, and

acyclic aliphatic groups of R² are unsubstituted or substituted by R^{d}.

also more preferred R² is CH═CH═CH₃, CH═C(CH₂)₃, or CH═C(CH₂)₄;

also more preferred R² is 2-furyl, 3-furyl, 2-methyl-3-furyl, or 3-methyl-2-furyl;

also most preferred R² is CH═CH═CH₃, CH═C(CH₂)₃, 2-furyl, 3-furyl, or 4-methyloxazol-5-yl;

also most preferred R² is CHOH—CHOH—C₆H₅, CHOH—CHOH-2-furyl, CHOH—CHOH—CH₃, or 4-methyl-5-oxazolyl.

also most preferred R² is CHOH—CHOH—C₆H₅, CHOH—CHOH-2-furyl, CHOH—CHOH—CH₃, or 4-methyloxazol-5-yl;

also most preferred R² is selected from R²-1 to R²-16 as shown below,

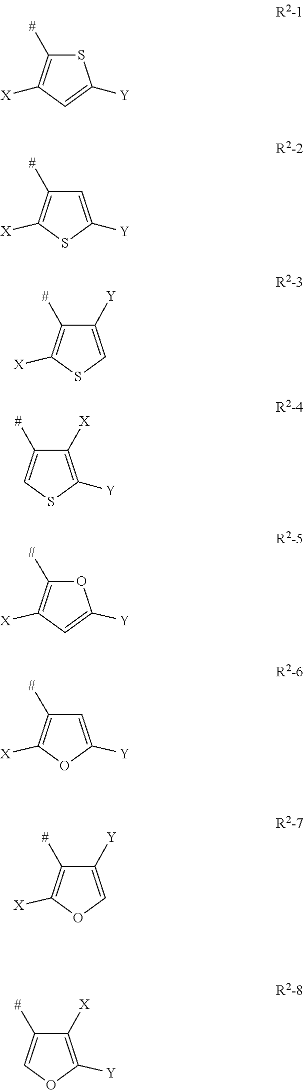

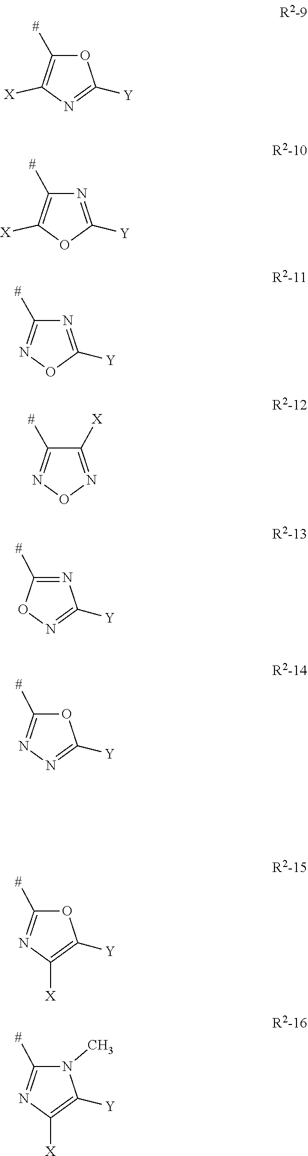

wherein # denotes attachment to the pyrimidine ring, X and Y denotes R^{c} which independently of each other are identical or different;

preferred R² is R²-1, R²-2, R²-3, R²-4, R²-5, R²-6, R²-7, or R²-8;

also preferred R² is R²-9, R²-10, R²-11, R²-13, R²-14, or R²-15;

more preferred R² is R²-9, R²-10, or R²-15;

most preferred R² is R²-9;

preferred X is H, halogen, CN, C₁-C₆-alkyl, C₁-C₆-haloalkyl, OH, C₁-C₆-alkoxy, C₁-C₆-haloalkoxy, or C₁-C₆-alkylthio;

particularly preferred X is H, halogen, CN, C₁-C₆-alkyl, OH, C₁-C₆-alkoxy, or C₁-C₆-alkylthio;

also particularly preferred X is H, halogen, CN, C₁-C₆-alkyl, C₁-C₆-haloalkyl, C₁-C₆-alkoxy, or C₁-C₆-haloalkoxy;

especially preferred X is H, halogen, CN, C₁-C₄-alkyl, OH, C₁-C₄-alkoxy, or C₁-C₄-alkylthio;

more preferred X is H, CH₃, C₂H₅, n-propyl, iso-propyl, iso-butyl, n-butyl, OH, OCH₃, SCH₃, F, Cl, Br, or I;

most preferred X is H, CH₃, C₂H₅, OH, or OCH₃;

also most preferred X is H, CH₃, C₂H₅, or SCH₃;

also most preferred X is H, CH₃, C₂H₅, F, C, Br, or I.

Preferred Y is H, halogen, CN, C₁-C₆-alkyl, C₁-C₆-haloalkyl, OH, C₁-C₆-alkoxy, C₁-C₆-haloalkoxy, or C₁-C₆-alkylthio;

particularly preferred Y is H, halogen, CN, C₁-C₆-alkyl, OH, C₁-C₆-alkoxy, or C₁-C₆-alkylthio;

also particularly preferred Y is H, halogen, CN, C₁-C₆-alkyl, C₁-C₆-haloalkyl, C₁-C₆-alkoxy, or C₁-C₆-haloalkoxy;

especially preferred Y is H, halogen, CN, C₁-C₄-alkyl, C₁-C₂-fluoroalkyl, OH, C₁-C₄-alkoxy, or C₁-C₄-alkylthio;

more preferred Y is H, CH₃, C₂H₅, n-propyl, iso-propyl, iso-butyl, n-butyl, 2-butyl, t-butyl, OH, OCH₃, SCH₃, F, Cl, Br, or I;

most preferred Y is H, CH₃, C₂H₅, n-propyl, iso-propyl, iso-butyl, n-butyl, 2-butyl, OH, or OCH₃;

also most preferred Y is H, CH₃, C₂H₅, n-propyl, iso-propyl, OH, OCH₃, or SCH₃;

also most preferred Y is H, CH₃, C₂H₅, n-propyl, iso-propyl, F, Cl, Br, or I.

Particularly preferred R² is 4-methyl-5-oxazolyl, 4-ethyl-5-oxazolyl, 2,4-dimethyl-5-oxazolyl, 2-ethyl-4-methyl-5-oxazolyl, 2-methyl-4-ethyl-5-oxazolyl, or 2,4-diethyl-5-oxazolyl.

Examples of particularly preferred R² are provided in Table R²-9, Table R²-10, and Table R²-15.

Table R²-9: examples of particularly preferred R² are R²-9.1 to R²-9.676 wherein R² is R²-9 and combinations of variables X and Y are as defined in each row of table R2, numbering of each compound e.g. R²-9.1 means R² is R²-9 wherein X and Y are as defined in row 1 of table R2;

Table R²-10: examples of particularly preferred R² are R²-10.1 to R²-10.676 wherein R² is R²-10 and combinations of variables X and Y are as defined in each row of table R2, numbering of each compound e.g. R²-10.1 means R² is R²-10 wherein X and Y are as defined in row 1 of table R2;

Table R²-15: examples of particularly preferred R² are R²-15.1 to R²-15.676 wherein R² is R²-15 and combinations of variables X and Y are as defined in each row of table R2, numbering of each compound e.g. R²-15.1 means R² is R²-15 wherein X and Y are as defined in row 1 of table R2.

TABLE R2

| row | X | Y |
|---|---|---|
| 1 | H | H |
| 2 | H | CH₃ |
| 3 | H | C₂H₅ |
| 4 | H | n-propyl |
| 5 | H | CH(CH₃)₂ |
| 6 | H | iso-butyl |
| 7 | H | n-butyl |
| 8 | H | 2-butyl |
| 9 | H | C(CH₃)₃ |
| 10 | H | OH |
| 11 | H | OCH₃ |
| 12 | H | SCH₃ |
| 13 | H | S(O)CH₃ |
| 14 | H | S(O)₂CH₃ |
| 15 | H | CN |
| 16 | H | F |
| 17 | H | Cl |
| 18 | H | Br |
| 19 | H | I |
| 20 | H | CH₂CF₃ |
| 21 | H | CF₂CF₃ |
| 22 | H | CF₂CH₃ |
| 23 | H | CF₃ |
| 24 | H | CF₂H |
| 25 | H | OCF₂H |
| 26 | H | OCF₃ |
| 27 | CH₃ | H |
| 28 | CH₃ | CH₃ |
| 29 | CH₃ | C₂H₅ |
| 30 | CH₃ | n-propyl |
| 31 | CH₃ | CH(CH₃)₂ |
| 32 | CH₃ | iso-butyl |
| 33 | CH₃ | n-butyl |
| 34 | CH₃ | 2-butyl |
| 35 | CH₃ | C(CH₃)₃ |
| 36 | CH₃ | OH |
| 37 | CH₃ | OCH₃ |
| 38 | CH₃ | SCH₃ |
| 39 | CH₃ | S(O)CH₃ |
| 40 | CH₃ | S(O)₂CH₃ |
| 41 | CH₃ | CN |
| 42 | CH₃ | F |
| 43 | CH₃ | Cl |
| 44 | CH₃ | Br |
| 45 | CH₃ | I |
| 46 | CH₃ | CH₂CF₃ |
| 47 | CH₃ | CF₂CF₃ |
| 48 | CH₃ | CF₂CH₃ |
| 49 | CH₃ | CF₃ |
| 50 | CH₃ | CF₂H |
| 51 | CH₃ | OCF₂H |
| 52 | CH₃ | OCF₃ |
| 53 | C₂H₅ | H |
| 54 | C₂H₅ | CH₃ |
| 55 | C₂H₅ | C₂H₅ |
| 56 | C₂H₅ | n-propyl |
| 57 | C₂H₅ | CH(CH₃)₂ |
| 58 | C₂H₅ | iso-butyl |
| 59 | C₂H₅ | n-butyl |
| 60 | C₂H₅ | 2-butyl |
| 61 | C₂H₅ | C(CH₃)₃ |
| 62 | C₂H₅ | OH |
| 63 | C₂H₅ | OCH₃ |
| 64 | C₂H₅ | SCH₃ |
| 65 | C₂H₅ | S(O)CH₃ |
| 66 | C₂H₅ | S(O)₂CH₃ |
| 67 | C₂H₅ | CN |
| 68 | C₂H₅ | F |
| 69 | C₂H₅ | Cl |
| 70 | C₂H₅ | Br |
| 71 | C₂H₅ | I |
| 72 | C₂H₅ | CH₂CF₃ |
| 73 | C₂H₅ | CF₂CF₃ |
| 74 | C₂H₅ | CF₂CH₃ |
| 75 | C₂H₅ | CF₃ |
| 76 | C₂H₅ | CF₂H |
| 77 | C₂H₅ | OCF₂H |
| 78 | C₂H₅ | OCF₃ |
| 79 | n-propyl | H |
| 80 | n-propyl | CH₃ |
| 81 | n-propyl | C₂H₅ |
| 82 | n-propyl | n-propyl |
| 83 | n-propyl | CH(CH₃)₂ |
| 84 | n-propyl | iso-butyl |
| 85 | n-propyl | n-butyl |
| 86 | n-propyl | 2-butyl |
| 87 | n-propyl | C(CH₃)₃ |
| 88 | n-propyl | OH |
| 89 | n-propyl | OCH₃ |
| 90 | n-propyl | SCH₃ |
| 91 | n-propyl | S(O)CH₃ |
| 92 | n-propyl | S(O)₂CH₃ |
| 93 | n-propyl | CN |
| 94 | n-propyl | F |
| 95 | n-propyl | Cl |
| 96 | n-propyl | Br |
| 97 | n-propyl | I |
| 98 | n-propyl | CH₂CF₃ |
| 99 | n-propyl | CF₂CF₃ |
| 100 | n-propyl | CF₂CH₃ |
| 101 | n-propyl | CF₃ |
| 102 | n-propyl | CF₂H |
| 103 | n-propyl | OCF₂H |
| 104 | n-propyl | OCF₃ |
| 105 | CH(CH₃)₂ | H |
| 106 | CH(CH₃)₂ | CH₃ |
| 107 | CH(CH₃)₂ | C₂H₅ |
| 108 | CH(CH₃)₂ | n-propyl |
| 109 | CH(CH₃)₂ | CH(CH₃)₂ |
| 110 | CH(CH₃)₂ | iso-butyl |
| 111 | CH(CH₃)₂ | n-butyl |
| 112 | CH(CH₃)₂ | 2-butyl |
| 113 | CH(CH₃)₂ | C(CH₃)₃ |
| 114 | CH(CH₃)₂ | OH |
| 115 | CH(CH₃)₂ | OCH₃ |
| 116 | CH(CH₃)₂ | SCH₃ |
| 117 | CH(CH₃)₂ | S(O)CH₃ |
| 118 | CH(CH₃)₂ | S(O)₂CH₃ |
| 119 | CH(CH₃)₂ | CN |
| 120 | CH(CH₃)₂ | F |
| 121 | CH(CH₃)₂ | Cl |
| 122 | CH(CH₃)₂ | Br |
| 123 | CH(CH₃)₂ | I |
| 124 | CH(CH₃)₂ | CH₂CF₃ |
| 125 | CH(CH₃)₂ | CF₂CF₃ |
| 126 | CH(CH₃)₂ | CF₂CH₃ |
| 127 | CH(CH₃)₂ | CF₃ |
| 128 | CH(CH₃)₂ | CF₂H |
| 129 | CH(CH₃)₂ | OCF₂H |
| 130 | CH(CH₃)₂ | OCF₃ |
| 131 | iso-butyl | H |
| 132 | iso-butyl | CH₃ |
| 133 | iso-butyl | C₂H₅ |
| 134 | iso-butyl | n-propyl |
| 135 | iso-butyl | CH(CH₃)₂ |
| 136 | iso-butyl | iso-butyl |
| 137 | iso-butyl | n-butyl |
| 138 | iso-butyl | 2-butyl |
| 139 | iso-butyl | C(CH₃)₃ |
| 140 | iso-butyl | OH |
| 141 | iso-butyl | OCH₃ |
| 142 | iso-butyl | SCH₃ |
| 143 | iso-butyl | S(O)CH₃ |
| 144 | iso-butyl | S(O)₂CH₃ |
| 145 | iso-butyl | CN |
| 146 | iso-butyl | F |
| 147 | iso-butyl | Cl |
| 148 | iso-butyl | Br |
| 149 | iso-butyl | I |
| 150 | iso-butyl | CH₂CF₃ |
| 151 | iso-butyl | CF₂CF₃ |
| 152 | iso-butyl | CF₂CH₃ |
| 153 | iso-butyl | CF₃ |
| 154 | iso-butyl | CF₂H |
| 155 | iso-butyl | OCF₂H |
| 156 | iso-butyl | OCF₃ |
| 157 | n-butyl | H |
| 158 | n-butyl | CH₃ |
| 159 | n-butyl | C₂H₅ |
| 160 | n-butyl | n-propyl |
| 161 | n-butyl | CH(CH₃)₂ |
| 162 | n-butyl | iso-butyl |
| 163 | n-butyl | n-butyl |
| 164 | n-butyl | 2-butyl |
| 165 | n-butyl | C(CH₃)₃ |
| 166 | n-butyl | OH |
| 167 | n-butyl | OCH₃ |
| 168 | n-butyl | SCH₃ |
| 169 | n-butyl | S(O)CH₃ |
| 170 | n-butyl | S(O)₂CH₃ |
| 171 | n-butyl | CN |
| 172 | n-butyl | F |
| 173 | n-butyl | Cl |
| 174 | n-butyl | Br |
| 175 | n-butyl | I |
| 176 | n-butyl | CH₂CF₃ |
| 177 | n-butyl | CF₂CF₃ |
| 178 | n-butyl | CF₂CH₃ |
| 179 | n-butyl | CF₃ |
| 180 | n-butyl | CF₂H |
| 181 | n-butyl | OCF₂H |
| 182 | n-butyl | OCF₃ |
| 183 | 2-butyl | H |
| 184 | 2-butyl | CH₃ |
| 185 | 2-butyl | C₂H₅ |
| 186 | 2-butyl | n-propyl |
| 187 | 2-butyl | CH(CH₃)₂ |
| 188 | 2-butyl | iso-butyl |
| 189 | 2-butyl | n-butyl |
| 190 | 2-butyl | 2-butyl |
| 191 | 2-butyl | C(CH₃)₃ |
| 192 | 2-butyl | OH |
| 193 | 2-butyl | OCH₃ |
| 194 | 2-butyl | SCH₃ |
| 195 | 2-butyl | S(O)CH₃ |
| 196 | 2-butyl | S(O)₂CH₃ |
| 197 | 2-butyl | CN |
| 198 | 2-butyl | F |
| 199 | 2-butyl | Cl |
| 200 | 2-butyl | Br |
| 201 | 2-butyl | I |
| 202 | 2-butyl | CH₂CF₃ |
| 203 | 2-butyl | CF₂CF₃ |
| 204 | 2-butyl | CF₂CH₃ |
| 205 | 2-butyl | CF₃ |
| 206 | 2-butyl | CF₂H |
| 207 | 2-butyl | OCF₂H |
| 208 | 2-butyl | OCF₃ |
| 209 | C(CH₃)₃ | H |
| 210 | C(CH₃)₃ | CH₃ |
| 211 | C(CH₃)₃ | C₂H₅ |
| 212 | C(CH₃)₃ | n-propyl |
| 213 | C(CH₃)₃ | CH(CH₃)₂ |
| 214 | C(CH₃)₃ | iso-butyl |
| 215 | C(CH₃)₃ | n-butyl |
| 216 | C(CH₃)₃ | 2-butyl |
| 217 | C(CH₃)₃ | C(CH₃)₃ |
| 218 | C(CH₃)₃ | OH |
| 219 | C(CH₃)₃ | OCH₃ |
| 220 | C(CH₃)₃ | SCH₃ |
| 221 | C(CH₃)₃ | S(O)CH₃ |
| 222 | C(CH₃)₃ | S(O)₂CH₃ |
| 223 | C(CH₃)₃ | CN |
| 224 | C(CH₃)₃ | F |
| 225 | C(CH₃)₃ | Cl |
| 226 | C(CH₃)₃ | Br |
| 227 | C(CH₃)₃ | I |
| 228 | C(CH₃)₃ | CH₂CF₃ |
| 229 | C(CH₃)₃ | CF₂CF₃ |
| 230 | C(CH₃)₃ | CF₂CH₃ |
| 231 | C(CH₃)₃ | CF₃ |
| 232 | C(CH₃)₃ | CF₂H |
| 233 | C(CH₃)₃ | OCF₂H |
| 234 | C(CH₃)₃ | OCF₃ |
| 235 | OH | H |
| 236 | OH | CH₃ |
| 237 | OH | C₂H₅ |
| 238 | OH | n-propyl |
| 239 | OH | CH(CH₃)₂ |
| 240 | OH | iso-butyl |
| 241 | OH | n-butyl |
| 242 | OH | 2-butyl |
| 243 | OH | C(CH₃)₃ |
| 244 | OH | OH |
| 245 | OH | OCH₃ |
| 246 | OH | SCH₃ |
| 247 | OH | S(O)CH₃ |
| 248 | OH | S(O)₂CH₃ |
| 249 | OH | CN |
| 250 | OH | F |
| 251 | OH | Cl |
| 252 | OH | Br |
| 253 | OH | I |
| 254 | OH | CH₂CF₃ |
| 255 | OH | CF₂CF₃ |
| 256 | OH | CF₂CH₃ |
| 257 | OH | CF₃ |
| 258 | OH | CF₂H |
| 259 | OH | OCF₂H |
| 260 | OH | OCF₃ |
| 261 | OCH₃ | H |
| 262 | OCH₃ | CH₃ |
| 263 | OCH₃ | C₂H₅ |
| 264 | OCH₃ | n-propyl |
| 265 | OCH₃ | CH(CH₃)₂ |
| 266 | OCH₃ | iso-butyl |
| 267 | OCH₃ | n-butyl |
| 268 | OCH₃ | 2-butyl |
| 269 | OCH₃ | C(CH₃)₃ |
| 270 | OCH₃ | OH |
| 271 | OCH₃ | OCH₃ |
| 272 | OCH₃ | SCH₃ |
| 273 | OCH₃ | S(O)CH₃ |
| 274 | OCH₃ | S(O)₂CH₃ |
| 275 | OCH₃ | CN |
| 276 | OCH₃ | F |
| 277 | OCH₃ | Cl |
| 278 | OCH₃ | Br |
| 279 | OCH₃ | I |
| 280 | OCH₃ | CH₂CF₃ |
| 281 | OCH₃ | CF₂CF₃ |
| 282 | OCH₃ | CF₂CH₃ |
| 283 | OCH₃ | CF₃ |
| 284 | OCH₃ | CF₂H |
| 285 | OCH₃ | OCF₂H |
| 286 | OCH₃ | OCF₃ |
| 287 | SCH₃ | H |
| 288 | SCH₃ | CH₃ |
| 289 | SCH₃ | C₂H₅ |
| 290 | SCH₃ | n-propyl |
| 291 | SCH₃ | CH(CH₃)₂ |
| 292 | SCH₃ | iso-butyl |
| 293 | SCH₃ | n-butyl |
| 294 | SCH₃ | 2-butyl |
| 295 | SCH₃ | C(CH₃)₃ |
| 296 | SCH₃ | OH |
| 297 | SCH₃ | OCH₃ |
| 298 | SCH₃ | SCH₃ |
| 299 | SCH₃ | S(O)CH₃ |
| 300 | SCH₃ | S(O)₂CH₃ |
| 301 | SCH₃ | CN |
| 302 | SCH₃ | F |
| 303 | SCH₃ | Cl |
| 304 | SCH₃ | Br |
| 305 | SCH₃ | I |
| 306 | SCH₃ | CH₂CF₃ |
| 307 | SCH₃ | CF₂CF₃ |
| 308 | SCH₃ | CF₂CH₃ |
| 309 | SCH₃ | CF₃ |
| 310 | SCH₃ | CF₂H |
| 311 | SCH₃ | OCF₂H |
| 312 | SCH₃ | OCF₃ |
| 313 | S(O)CH₃ | H |
| 314 | S(O)CH₃ | CH₃ |
| 315 | S(O)CH₃ | C₂H₅ |
| 316 | S(O)CH₃ | n-propyl |
| 317 | S(O)CH₃ | CH(CH₃)₂ |
| 318 | S(O)CH₃ | iso-butyl |
| 319 | S(O)CH₃ | n-butyl |
| 320 | S(O)CH₃ | 2-butyl |
| 321 | S(O)CH₃ | C(CH₃)₃ |
| 322 | S(O)CH₃ | OH |
| 323 | S(O)CH₃ | OCH₃ |
| 324 | S(O)CH₃ | SCH₃ |
| 325 | S(O)CH₃ | S(O)CH₃ |
| 326 | S(O)CH₃ | S(O)₂CH₃ |
| 327 | S(O)CH₃ | CN |
| 328 | S(O)CH₃ | F |
| 329 | S(O)CH₃ | Cl |
| 330 | S(O)CH₃ | Br |
| 331 | S(O)CH₃ | I |
| 332 | S(O)CH₃ | CH₂CF₃ |
| 333 | S(O)CH₃ | CF₂CF₃ |
| 334 | S(O)CH₃ | CF₂CH₃ |
| 335 | S(O)CH₃ | CF₃ |
| 336 | S(O)CH₃ | CF₂H |
| 337 | S(O)CH₃ | OCF₂H |
| 338 | S(O)CH₃ | OCF₃ |
| 339 | S(O)₂CH₃ | H |
| 340 | S(O)₂CH₃ | CH₃ |
| 341 | S(O)₂CH₃ | C₂H₅ |
| 342 | S(O)₂CH₃ | n-propyl |
| 343 | S(O)₂CH₃ | CH(CH₃)₂ |
| 344 | S(O)₂CH₃ | iso-butyl |
| 345 | S(O)₂CH₃ | n-butyl |
| 346 | S(O)₂CH₃ | 2-butyl |
| 347 | S(O)₂CH₃ | C(CH₃)₃ |
| 348 | S(O)₂CH₃ | OH |
| 349 | S(O)₂CH₃ | OCH₃ |
| 350 | S(O)₂CH₃ | SCH₃ |
| 351 | S(O)₂CH₃ | S(O)CH₃ |
| 352 | S(O)₂CH₃ | S(O)₂CH₃ |
| 353 | S(O)₂CH₃ | CN |
| 354 | S(O)₂CH₃ | F |
| 355 | S(O)₂CH₃ | Cl |
| 356 | S(O)₂CH₃ | Br |
| 357 | S(O)₂CH₃ | I |
| 358 | S(O)₂CH₃ | CH₂CF₃ |
| 359 | S(O)₂CH₃ | CF₂CF₃ |
| 360 | S(O)₂CH₃ | CF₂CH₃ |
| 361 | S(O)₂CH₃ | CF₃ |
| 362 | S(O)₂CH₃ | CF₂H |
| 363 | S(O)₂CH₃ | OCF₂H |
| 364 | S(O)₂CH₃ | OCF₃ |
| 365 | CN | H |
| 366 | CN | CH₃ |
| 367 | CN | C₂H₅ |
| 368 | CN | n-propyl |
| 369 | CN | CH(CH₃)₂ |
| 370 | CN | iso-butyl |
| 371 | CN | n-butyl |
| 372 | CN | 2-butyl |
| 373 | CN | C(CH₃)₃ |
| 374 | CN | OH |
| 375 | CN | OCH₃ |
| 376 | CN | SCH₃ |
| 377 | CN | S(O)CH₃ |
| 378 | CN | S(O)₂CH₃ |
| 379 | CN | CN |
| 380 | CN | F |
| 381 | CN | Cl |
| 382 | CN | Br |
| 383 | CN | I |
| 384 | CN | CH₂CF₃ |
| 385 | CN | CF₂CF₃ |
| 386 | CN | CF₂CH₃ |
| 387 | CN | CF₃ |
| 388 | CN | CF₂H |
| 389 | CN | OCF₂H |
| 390 | CN | OCF₃ |
| 391 | F | H |
| 392 | F | CH₃ |
| 393 | F | C₂H₅ |
| 394 | F | n-propyl |
| 395 | F | CH(CH₃)₂ |
| 396 | F | iso-butyl |
| 397 | F | n-butyl |
| 398 | F | 2-butyl |
| 399 | F | C(CH₃)₃ |
| 400 | F | OH |
| 401 | F | OCH₃ |
| 402 | F | SCH₃ |
| 403 | F | S(O)CH₃ |
| 404 | F | S(O)₂CH₃ |
| 405 | F | CN |
| 406 | F | F |
| 407 | F | Cl |
| 408 | F | Br |
| 409 | F | I |
| 410 | F | CH₂CF₃ |
| 411 | F | CF₂CF₃ |
| 412 | F | CF₂CH₃ |
| 413 | F | CF₃ |
| 414 | F | CF₂H |
| 415 | F | OCF₂H |
| 416 | F | OCF₃ |
| 417 | Cl | H |
| 418 | Cl | CH₃ |
| 419 | Cl | C₂H₅ |
| 420 | Cl | n-propyl |
| 421 | Cl | CH(CH₃)₂ |
| 422 | Cl | iso-butyl |
| 423 | Cl | n-butyl |
| 424 | Cl | 2-butyl |
| 425 | Cl | C(CH₃)₃ |
| 426 | Cl | OH |
| 427 | Cl | OCH₃ |
| 428 | Cl | SCH₃ |
| 429 | Cl | S(O)CH₃ |
| 430 | Cl | S(O)₂CH₃ |
| 431 | Cl | CN |
| 432 | Cl | F |
| 433 | Cl | Cl |
| 434 | Cl | Br |
| 435 | Cl | I |
| 436 | Cl | CH₂CF₃ |
| 437 | Cl | CF₂CF₃ |
| 438 | Cl | CF₂CH₃ |
| 439 | Cl | CF₃ |
| 440 | Cl | CF₂H |
| 441 | Cl | OCF₂H |
| 442 | Cl | OCF₃ |
| 443 | Br | H |
| 444 | Br | CH₃ |
| 445 | Br | C₂H₅ |
| 446 | Br | n-propyl |
| 447 | Br | CH(CH₃)₂ |
| 448 | Br | iso-butyl |
| 449 | Br | n-butyl |
| 450 | Br | 2-butyl |
| 451 | Br | C(CH₃)₃ |
| 452 | Br | OH |
| 453 | Br | OCH₃ |
| 454 | Br | SCH₃ |
| 455 | Br | S(O)CH₃ |
| 456 | Br | S(O)₂CH₃ |
| 457 | Br | CN |
| 458 | Br | F |
| 459 | Br | Cl |
| 460 | Br | Br |
| 461 | Br | I |
| 462 | Br | CH₂CF₃ |
| 463 | Br | CF₂CF₃ |
| 464 | Br | CF₂CH₃ |
| 465 | Br | CF₃ |
| 466 | Br | CF₂H |
| 467 | Br | OCF₂H |
| 468 | Br | OCF₃ |
| 469 | I | H |
| 470 | I | CH₃ |
| 471 | I | C₂H₅ |
| 472 | I | n-propyl |
| 473 | I | CH(CH₃)₂ |
| 474 | I | iso-butyl |
| 475 | I | n-butyl |
| 476 | I | 2-butyl |
| 477 | I | C(CH₃)₃ |
| 478 | I | OH |
| 479 | I | OCH₃ |
| 480 | I | SCH₃ |
| 481 | I | S(O)CH₃ |
| 482 | I | S(O)₂CH₃ |
| 483 | I | CN |
| 484 | I | F |
| 485 | I | Cl |
| 486 | I | Br |
| 487 | I | I |
| 488 | I | CH₂CF₃ |
| 489 | I | CF₂CF₃ |
| 490 | I | CF₂CH₃ |
| 491 | I | CF₃ |
| 492 | I | CF₂H |
| 493 | I | OCF₂H |
| 494 | I | OCF₃ |
| 495 | CH₂CF₃ | H |
| 496 | CH₂CF₃ | CH₃ |
| 497 | CH₂CF₃ | C₂H₅ |
| 498 | CH₂CF₃ | n-propyl |
| 499 | CH₂CF₃ | CH(CH₃)₂ |
| 500 | CH₂CF₃ | iso-butyl |
| 501 | CH₂CF₃ | n-butyl |
| 502 | CH₂CF₃ | 2-butyl |
| 503 | CH₂CF₃ | C(CH₃)₃ |
| 504 | CH₂CF₃ | OH |
| 505 | CH₂CF₃ | OCH₃ |
| 506 | CH₂CF₃ | SCH₃ |
| 507 | CH₂CF₃ | S(O)CH₃ |
| 508 | CH₂CF₃ | S(O)₂CH₃ |
| 509 | CH₂CF₃ | CN |
| 510 | CH₂CF₃ | F |
| 511 | CH₂CF₃ | Cl |
| 512 | CH₂CF₃ | Br |
| 513 | CH₂CF₃ | I |
| 514 | CH₂CF₃ | CH₂CF₃ |
| 515 | CH₂CF₃ | CF₂CF₃ |
| 516 | CH₂CF₃ | CF₂CH₃ |
| 517 | CH₂CF₃ | CF₃ |
| 518 | CH₂CF₃ | CF₂H |
| 519 | CH₂CF₃ | OCF₂H |
| 520 | CH₂CF₃ | OCF₃ |
| 521 | CF₂CF₃ | H |
| 522 | CF₂CF₃ | CH₃ |
| 523 | CF₂CF₃ | C₂H₅ |
| 524 | CF₂CF₃ | n-propyl |
| 525 | CF₂CF₃ | CH(CH₃)₂ |
| 526 | CF₂CF₃ | iso-butyl |
| 527 | CF₂CF₃ | n-butyl |
| 528 | CF₂CF₃ | 2-butyl |
| 529 | CF₂CF₃ | C(CH₃)₃ |
| 530 | CF₂CF₃ | OH |
| 531 | CF₂CF₃ | OCH₃ |
| 532 | CF₂CF₃ | SCH₃ |
| 533 | CF₂CF₃ | S(O)CH₃ |
| 534 | CF₂CF₃ | S(O)₂CH₃ |
| 535 | CF₂CF₃ | CN |
| 536 | CF₂CF₃ | F |
| 537 | CF₂CF₃ | Cl |
| 538 | CF₂CF₃ | Br |
| 539 | CF₂CF₃ | I |
| 540 | CF₂CF₃ | CH₂CF₃ |
| 541 | CF₂CF₃ | CF₂CF₃ |
| 542 | CF₂CF₃ | CF₂CH₃ |
| 543 | CF₂CF₃ | CF₃ |
| 544 | CF₂CF₃ | CF₂H |
| 545 | CF₂CF₃ | OCF₂H |
| 546 | CF₂CF₃ | OCF₃ |
| 547 | CF₂CH₃ | H |
| 548 | CF₂CH₃ | CH₃ |
| 549 | CF₂CH₃ | C₂H₅ |
| 550 | CF₂CH₃ | n-propyl |
| 551 | CF₂CH₃ | CH(CH₃)₂ |
| 552 | CF₂CH₃ | iso-butyl |
| 553 | CF₂CH₃ | n-butyl |
| 554 | CF₂CH₃ | 2-butyl |
| 555 | CF₂CH₃ | C(CH₃)₃ |
| 556 | CF₂CH₃ | OH |
| 557 | CF₂CH₃ | OCH₃ |
| 558 | CF₂CH₃ | SCH₃ |
| 559 | CF₂CH₃ | S(O)CH₃ |
| 560 | CF₂CH₃ | S(O)₂CH₃ |
| 561 | CF₂CH₃ | CN |
| 562 | CF₂CH₃ | F |
| 563 | CF₂CH₃ | Cl |
| 564 | CF₂CH₃ | Br |
| 565 | CF₂CH₃ | I |
| 566 | CF₂CH₃ | CH₂CF₃ |
| 567 | CF₂CH₃ | CF₂CF₃ |
| 568 | CF₂CH₃ | CF₂CH₃ |
| 569 | CF₂CH₃ | CF₃ |
| 570 | CF₂CH₃ | CF₂H |
| 571 | CF₂CH₃ | OCF₂H |
| 572 | CF₂CH₃ | OCF₃ |
| 573 | CF₃ | H |
| 574 | CF₃ | CH₃ |
| 575 | CF₃ | C₂H₅ |
| 576 | CF₃ | n-propyl |
| 577 | CF₃ | CH(CH₃)₂ |
| 578 | CF₃ | iso-butyl |
| 579 | CF₃ | n-butyl |
| 580 | CF₃ | 2-butyl |
| 581 | CF₃ | C(CH₃)₃ |
| 582 | CF₃ | OH |
| 583 | CF₃ | OCH₃ |
| 584 | CF₃ | SCH₃ |
| 585 | CF₃ | S(O)CH₃ |
| 586 | CF₃ | S(O)₂CH₃ |
| 587 | CF₃ | CN |
| 588 | CF₃ | F |
| 589 | CF₃ | Cl |
| 590 | CF₃ | Br |
| 591 | CF₃ | I |
| 592 | CF₃ | CH₂CF₃ |
| 593 | CF₃ | CF₂CF₃ |
| 594 | CF₃ | CF₂CH₃ |
| 595 | CF₃ | CF₃ |
| 596 | CF₃ | CF₂H |
| 597 | CF₃ | OCF₂H |
| 598 | CF₃ | OCF₃ |
| 599 | CF₂H | H |
| 600 | CF₂H | CH₃ |
| 601 | CF₂H | C₂H₅ |
| 602 | CF₂H | n-propyl |
| 603 | CF₂H | CH(CH₃)₂ |
| 604 | CF₂H | iso-butyl |
| 605 | CF₂H | n-butyl |
| 606 | CF₂H | 2-butyl |
| 607 | CF₂H | C(CH₃)₃ |
| 608 | CF₂H | OH |
| 609 | CF₂H | OCH₃ |
| 610 | CF₂H | SCH₃ |
| 611 | CF₂H | S(O)CH₃ |
| 612 | CF₂H | S(O)₂CH₃ |
| 613 | CF₂H | CN |
| 614 | CF₂H | F |
| 615 | CF₂H | Cl |
| 616 | CF₂H | Br |
| 617 | CF₂H | I |
| 618 | CF₂H | CH₂CF₃ |
| 619 | CF₂H | CF₂CF₃ |
| 620 | CF₂H | CF₂CH₃ |
| 621 | CF₂H | CF₃ |
| 622 | CF₂H | CF₂H |
| 623 | CF₂H | OCF₂H |
| 624 | CF₂H | OCF₃ |
| 625 | OCF₂H | H |
| 626 | OCF₂H | CH₃ |
| 627 | OCF₂H | C₂H₅ |
| 628 | OCF₂H | n-propyl |
| 629 | OCF₂H | CH(CH₃)₂ |
| 630 | OCF₂H | iso-butyl |
| 631 | OCF₂H | n-butyl |
| 632 | OCF₂H | 2-butyl |
| 633 | OCF₂H | C(CH₃)₃ |
| 634 | OCF₂H | OH |
| 635 | OCF₂H | OCH₃ |
| 636 | OCF₂H | SCH₃ |
| 637 | OCF₂H | S(O)CH₃ |
| 638 | OCF₂H | S(O)₂CH₃ |
| 639 | OCF₂H | CN |
| 640 | OCF₂H | F |
| 641 | OCF₂H | Cl |
| 642 | OCF₂H | Br |
| 643 | OCF₂H | I |
| 644 | OCF₂H | CH₂CF₃ |
| 645 | OCF₂H | CF₂CF₃ |
| 646 | OCF₂H | CF₂CH₃ |
| 647 | OCF₂H | CF₃ |
| 648 | OCF₂H | CF₂H |
| 649 | OCF₂H | OCF₂H |
| 650 | OCF₂H | OCF₃ |
| 651 | OCF₃ | H |
| 652 | OCF₃ | CH₃ |
| 653 | OCF₃ | C₂H₅ |
| 654 | OCF₃ | n-propyl |
| 655 | OCF₃ | CH(CH₃)₂ |
| 656 | OCF₃ | iso-butyl |
| 657 | OCF₃ | n-butyl |
| 658 | OCF₃ | 2-butyl |
| 659 | OCF₃ | C(CH₃)₃ |
| 660 | OCF₃ | OH |
| 661 | OCF₃ | OCH₃ |
| 662 | OCF₃ | SCH₃ |
| 663 | OCF₃ | S(O)CH₃ |
| 664 | OCF₃ | S(O)₂CH₃ |
| 665 | OCF₃ | CN |
| 666 | OCF₃ | F |
| 667 | OCF₃ | Cl |
| 668 | OCF₃ | Br |
| 669 | OCF₃ | I |
| 670 | OCF₃ | CH₂CF₃ |
| 671 | OCF₃ | CF₂CF₃ |
| 672 | OCF₃ | CF₂CH₃ |
| 673 | OCF₃ | CF₃ |
| 674 | OCF₃ | CF₂H |
| 675 | OCF₃ | OCF₂H |
| 676 | OCF₃ | OCF₃ |

Preferred A is OR³ or NR^{3A};

most preferred A is OR³;

also most preferred A is NR^{3A}.

Preferred Z is 6-membered heteroaryl ring, preferably triazine, pyrimidine, or pyridine;

particularly preferred Z is pyrimidine or pyridine;

especially preferred Z is pyridine.

Also preferred Z is 5-membered heteroaryl ring, preferably thiadiazole, oxadiazole, triazole, thiazole, isothiazole, oxazole, isoxazole, pyrazole, imidazole, thiophene, furan, or pyrrole;

particularly preferred Z is thiazole, isothiazole, oxazole, isoxazole, pyrazole, imidazole, thiophene, furan, or pyrrole;

especially preferred Z is thiophene, furan, or pyrrole;

particularly preferred Z is selected from below groups A to G,

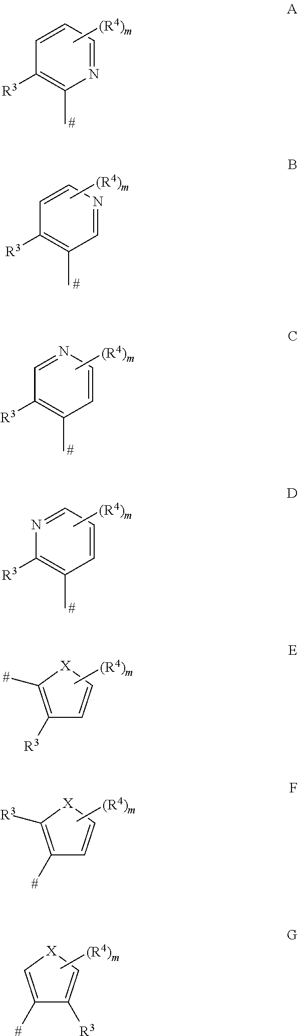

wherein
R³ is halogen, CHO, CN, C₁-C₆-alkyl, C₁-C₆-haloalkyl, or C₁-C₆-alkoxy;
m is 0 or 1;
R⁴ is halogen, CHO, CN, C₁-C₆-alkyl, C₁-C₆-haloalkyl, or C₁-C₆-alkoxy;
X is O, S, or NR^{3A}; and
# denotes the point of attachment to the pyrimidine ring.

Preferred R³ is halogen, CN, NO₂, C₁-C₆-alkyl, C₁-C₆-haloalkyl, C₁-C₆-alkoxy, or C₃-C₆-cycloalkyl;

also preferred R³ is halogen, CN, C₁-C₆-alkyl, C₁-C₆-haloalkyl, or C₁-C₆-alkoxy,

particularly preferred R³ is halogen, CN, C₁-C₆-alkyl, or C₁-C₆-alkoxy;

especially preferred halogen, or CH₃;

also especially preferred R³ is halogen;

more preferred R³ is C, Br, or I;

most preferred R³ is C or Br.

Preferred R^{3A} is H, C₁-C₆-alkyl, C₁-C₆-haloalkyl, C₁-C₆-alkylcarbonyl, C₃-C₆-alkenyl, C₃-C₆-haloalkenyl, C₃-C₆-alkenyl, C₃-C₆-haloalkenyl, or C₃-C₆-cycloalkyl;

also preferred R^{3A} is H, C₁-C₆-alkyl, C₁-C₆-haloalkyl, or C₁-C₆-alkylcarbonyl;

particularly preferred R^{3A} is H, C₁-C₆-alkyl, or C₁-C₆-alkylcarbonyl;

especially preferred R^{3A} is H, or C₁-C₆-alkyl;

most preferred R^{3A} is H, or CH₃.

Preferred R⁴ is halogen, CN, C₁-C₆-alkyl, C₁-C₆-haloalkyl, or C₁-C₆-alkoxy;

particularly preferred R⁴ is halogen, C₁-C₆-haloalkyl, or C₁-C₆-alkyl;

especially preferred R⁴ is halogen;

also especially preferred R⁴ is C₁-C₆-haloalkyl, or C₁-C₆-alkyl;

more preferred R⁴ is F, Cl, CHF₂, CF₃, CH₃, or C₂H₅;

most preferred R⁴ is F;

also most preferred R⁴ is CH₃;

also most preferred R⁴ is Cl.

also most preferred R⁴ is CF₃.

Preferred m is 0, 1, or 2;

more preferred m is 0 or 1;

most preferred m is 0.

also most preferred m is 1.

Also preferred are the pyrimidine compounds of formula (I), and their use as herbicide, wherein

R¹ is preferably C₁-C₆-alkyl, C₁-C₆-alkoxy, or C₃-C₆-cycloalkyl, wherein the cycloalkyl substituent is unsubstituted;

particularly preferred R¹ is C₃-C₆-cycloalkyl, wherein the cycloalkyl substituent is unsubstituted;

R² is preferably C₂-C₆-alkenyl, C₃-C₆-cycloalkyl-C₁-C₆-alkylidenyl, 5- or 6-membered heteroaryl, C₁-C₆-hydroxyalkyl, or C₂-C₆-dihydroxyalkyl;

particularly preferred R² is C₂-C₆-alkenyl, 5- or 6-membered heteroaryl, or C₁-C₆-hydroxyalkyl;

also particularly preferred R² is C₃-C₆-cycloalkyl-C₁-C₆-alkylidenyl, C₂-C₆-dihydroxyalkyl or 5- or 6-membered heteroaryl;

more preferred R² is CH═CH═CH₃, CH═C(CH₂)₃, or CH═C(CH₂)₄;

also more preferred R² is 2-furyl, 3-furyl, 2-methyl-3-furyl, 3-methyl-2-furyl, 4-methyloxazol-5-yl, CHOH—CHOH—C₆H₅, or CHOH—CHOH-2-furyl;

most preferred R² is CH═CH═CH₃, CH═C(CH₂)₃, 2-furyl, 3-furyl, CHOH—CHOH—C₆H₅, CHOH—CHOH-2-furyl, or 4-methyl-5-oxazolyl.

wherein acyclic aliphatic groups of R² are unsubstituted or substituted by R^{d}.

A is preferably CR³ or NR^{3A};

particularly preferred A is CR³;

also particularly preferred A is NR^{3A};

Z is preferably pyridine, pyrrole, furan, or thiophene;

particularly preferred Z is pyridine;

also particularly preferred Z is pyrrole, furan, or thiophene;

more preferred Z is pyridine, furan, or thiophene;

most preferred Z is selected from groups A to G, as defined above;

specially preferred Z is A;

also specially preferred Z is C;

R³ is preferably halogen, CN, C₁-C₆-alkyl, C₁-C₆-haloalkyl, or C₁-C₆-alkoxy;

particularly preferred R³ is halogen or CH₃;

R^{3A} is preferably H or C₁-C₆-alkyl;

particularly preferred R^{3A} is H or CH₃;

m is preferably 0 or 1;

R⁴ is preferably halogen or CF₃.

Also preferred are the pyrimidine compounds of formula (I), wherein

R¹ is c-C₃H₅;

R² is C₁-C₆-alkyl, C₂-C₆-alkenyl, C₃-C₆-cycloalkyl-C₁-C₆-alkyl, C₃-C₆-cycloalkenyl-C₁-C₆-alkyl, C₃-C₆-cycloalkyl-C₁-C₆-alkylidenyl, C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-alkyl, C₁-C₆-alkoxycarbonyl-C₁-C₆-haloalkyl, or 5-membered heteroaryl;

wherein OH groups of R² are unsubstituted or substituted by R^{b},

cyclic groups of R² are unsubstituted or substituted by R^{c}, and

acyclic aliphatic groups of R² are unsubstituted or substituted by R^{d};

R^{b} is C₁-C₆-alkyl;

R^{c} is C₁-C₆-alkyl or OH;

R^{d} is phenyl or 5- or 6-membered heteroaryl;

wherein the substituent R^{d} is unsubstituted or substituted by R^{e};

R^{e} is halogen, CN, NO₂, C₁-C₆-alkyl, C₁-C₆-haloalkyl, OH, C₁-C₆-alkoxy, C₁-C₆-haloalkoxy, C₁-C₆-alkylsulfonyl;

Z is A, E, G, or F;

X is S;

R³ is Cl, Br, F, I, CH₃, or OCF₃;

m is 0 or 1;

R⁴ is Br.

Also preferred are the pyrimidine compounds of formula (I), wherein

R¹ is c-C₃H₅;

R² is C₁-C₆-alkyl, C₂-C₆-alkenyl, C₃-C₆-cycloalkyl-C₁-C₆-alkyl, C₃-C₆-cycloalkenyl-C₁-C₆-alkyl, C₃-C₆-cycloalkyl-C₁-C₆-alkylidenyl, C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-alkyl, C₁-C₆-alkoxycarbonyl-C₁-C₆-haloalkyl, or 5-membered heteroaryl;

wherein OH groups of R² are unsubstituted or substituted by R^{b},

cyclic groups of R² are unsubstituted or substituted by R^{c}, and

acyclic aliphatic groups of R² are unsubstituted or substituted by R^{d};

R^{b} is C₁-C₆-alkyl;

R^{c} is C₁-C₆-alkyl or OH;

R^{d} is phenyl or 5- or 6-membered heteroaryl;

wherein the substituent R^{d} is unsubstituted or substituted by R^{e};

R^{e} is halogen, CN, NO₂, C₁-C₆-alkyl, C₁-C₆-haloalkyl, OH, C₁-C₆-alkoxy, C₁-C₆-haloalkoxy, C₁-C₆-alkylsulfonyl;

Z is A or C;

R³ is C, Br, F, I, CH₃, or OCF₃;

m is 0 or 1;

R⁴ is halogen, preferably Br.

Also preferred are the pyrimidine compounds of formula (I), wherein

R¹ is c-C₃H₅;

R² is R²-9, R²-10 or R²-15, preferably R²-9;

Z is A or C;

R³ is R³ is halogen, C₁-C₆-alkyl, C₁-C₆-haloalkyl, C₁-C₆-alkoxy, or C₁-C₆-haloalkoxy; preferrably C₁-C₆-alkyl, C₁-C₆-haloalkyl, or C₁-C₆-haloalkoxy; more preferrably Cl, Br, F, I, CH₃, or OCF₃;

m is 0 or 1;

R⁴ is F, Br, Cl, CHF₂, CH₃, CF₃, or C₂H₅.

Also preferred are the pyrimidine compounds of formula (I.1) (corresponds to pyrimidine compounds of formula (I) wherein R² is CH═CH═CH₃), and their use as herbicide,

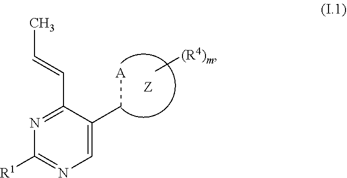

wherein the dotted line ( [symbol] ) is a single bond or a double bond;

R¹ is C₃-C₆-cycloalkyl, C₁-C₆-alkyl, or C₁-C₆-alkoxy;

A is CR³ or NR^{3A};

R³ is halogen, C₁-C₆-alkyl, C₁-C₆-haloalkyl, or C₁-C₆-alkoxy;

R^{3A} is H or C₁-C₆-alkyl;

Z is pyridine, thiophene, furan, or pyrrol;

m is 0 or 1;

R⁴ is F, Cl, CHF₂, CF₃, CH₃, or C₂H₅.

Also preferred are the pyrimidine compounds of formula (I.2) (corresponds to pyrimidine compounds of formula (I) wherein R² is CH═C(CH₂)₃), and their use as herbicide,

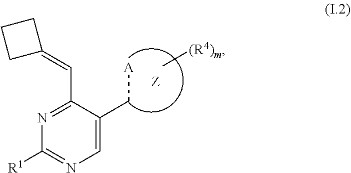

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.3) (corresponds to pyrimidine compounds of formula (I) wherein R² is CH═C(CH₂)₄), and their use as herbicide,

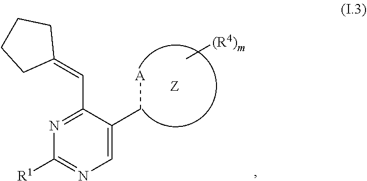

wherein the dotted line ( [symbol] ) is single bond or double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.4) (corresponds to pyrimidine compounds of formula (I) wherein R² is 2-furyl), and their use as herbicide,

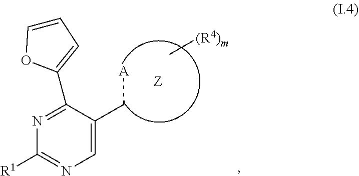

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.5) (corresponds to pyrimidine compounds of formula (I) wherein R² is 3-furyl), and their use as herbicide,

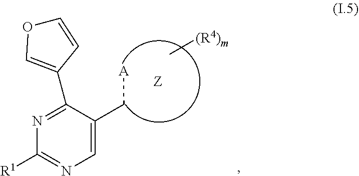

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.6) (corresponds to pyrimidine compounds of formula (I) wherein R² is 3-methyl-2-furyl), and their use as herbicide,

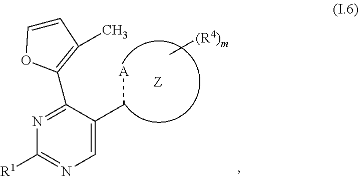

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.7) (corresponds to pyrimidine compounds of formula (I) wherein R² is 2-methyl-3-furyl), and their use as herbicide,

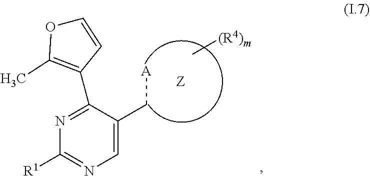

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.8) (corresponds to pyrimidine compounds of formula (I) wherein R² is CHOH—CHOH—C₆H₅), and their use as herbicide,

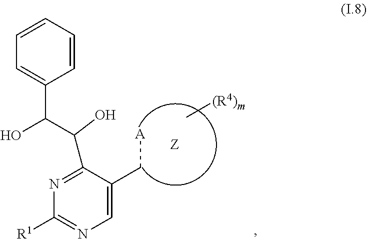

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.9) (corresponds to pyrimidine compounds of formula (I) wherein R² is CHOH—CHOH-2-furyl), and their use as herbicide,

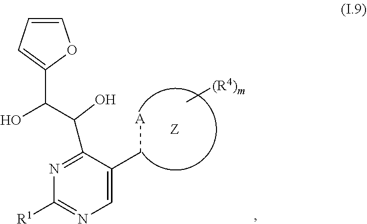

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅.

Also preferred are the pyrimidine compounds of formula (I.10) (corresponds to pyrimidine compounds of formula (I) wherein R² is 4-methyl-5-oxazolyl), and their use as herbicide,

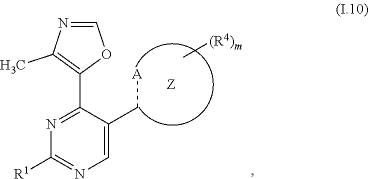

wherein the dotted line ( [symbol] ) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Cl, CHF₂, CH₃, CF₃, or C₂H₅

Also preferred are the pyrimidine compounds of formula (I.11) (corresponds to pyrimidine compounds of formula (I) wherein R² is R²-9), and their use as herbicide,

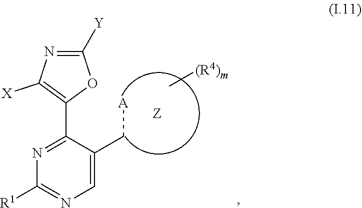

wherein the dotted line ( [symbol] ) is a single bond or a double bond;

X and Y independently are selected from H, CH₃, C₂H₅, n-propyl, iso-propyl, iso-butyl, n-butyl, 2-butyl, t-butyl, OH, OCH₃, SCH₃, S(O)CH₃, S(O)₂CH₃, CN, F, C, Br, I, CH₂CF₃, CF₂CF₃, CF₂CH₃, CF₃, CF₂H, OCF₂H, and OCF₃;

R¹ is C₃-C₆-cycloalkyl, C₁-C₆-alkyl, or C₁-C₆-alkoxy; preferably c-C₃H₅;

A is CR³;

R³ is halogen, C₁-C₆-alkyl, C₁-C₆-haloalkyl, or C₁-C₆-alkoxy;

Z is pyridine, thiophene, furan, or pyrrol;

m is 0 or 1;

R⁴ is F, Br, C, CHF₂, CH₃, CF₃, or C₂H₅; preferably F, Cl, CHF₂, CH₃, CF₃, or C₂H₅; also preferably Br.

Also preferred are the pyrimidine compounds of formula (I.11.A) (corresponds to pyrimidine compounds of formula (I), wherein R² is R²-9 and Z is A) and their use as herbicide,

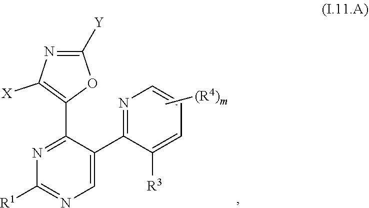

wherein

X and Y independently are selected from H, CH₃, C₂H₅, n-propyl, iso-propyl, iso-butyl, n-butyl, 2-butyl, t-butyl, OH, OCH₃, SCH₃, S(O)CH₃, S(O)₂CH₃, CN, F, Cl, Br, I, CH₂CF₃, CF₂CF₃, CF₂CH₃, CF₃, CF₂H, OCF₂H, and OCF₃;

R¹ is C₃-C₆-cycloalkyl, C₁-C₆-alkyl, or C₁-C₆-alkoxy; preferably c-C₃H₅;

R³ is halogen, C₁-C₆-alkyl, C₁-C₆-haloalkyl, or C₁-C₆-alkoxy;

m is 0 or 1;

R⁴ is F, Br, Cl, CHF₂, CH₃, CF₃, or C₂H.

Also preferred are the pyrimidine compounds of formula (I.11.C) (corresponds to pyrimidine compounds of formula (I), wherein R² is R²-9 and Z is C) and their use as herbicide,

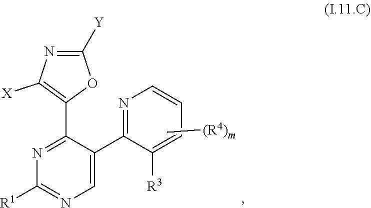

wherein

X and Y independently are selected from H, CH₃, C₂H₅, n-propyl, iso-propyl, iso-butyl, n-butyl, 2-butyl, t-butyl, OH, OCH₃, SCH₃, S(O)CH₃, S(O)₂CH₃, CN, F, C, Br, I, CH₂CF₃, CF₂CF₃, CF₂CH₃, CF₃, CF₂H, OCF₂H, and OCF₃;

R¹ is C₃-C₆-cycloalkyl, C₁-C₆-alkyl, or C₁-C₆-alkoxy; preferably c-C₃H₅;

R³ is halogen, C₁-C₆-alkyl, C₁-C₆-haloalkyl, or C₁-C₆-alkoxy;

m is 0 or 1;

R⁴ is F, Br, C, CHF₂, CH₃, CF₃, or C₂H.

Particular preference is given to the pyrimidine compounds of formula I.A to I.G (corresponds to pyrimidine compounds of formula (I)), and their use as herbicide, wherein X is O, NR^{3A}, or S.

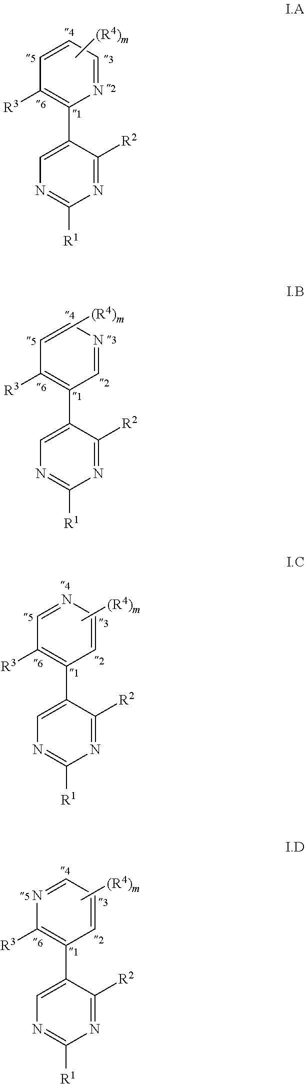

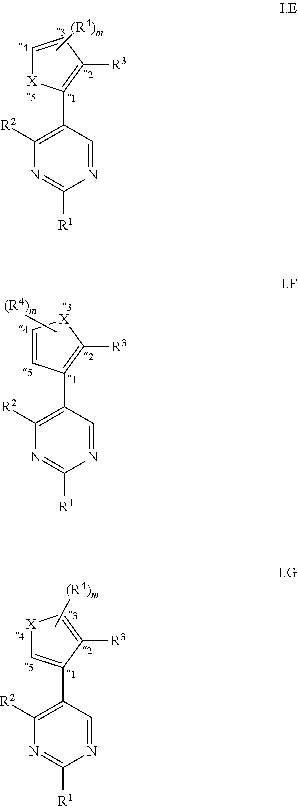

Preferred compounds of formula I, and their use as herbicide, are the compounds of the formulae I.A to I.G wherein

R¹ is C₂H₅, c-C₃H₅, c-C₄H₇, or OCH₃;

R² is CH═CH═CH₃, CH═C(CH₂)₃, CH═C(CH₂)₄, 2-furyl, 3-furyl, 4-methyl-2-fury, 2-methyl-3-furyl CHOH—CHOH—C₆H₅, CHOH—CHOH-2-furyl or 4-methyl-5-oxazolyl;

R³ is CH₃, OCH₃, Cl, Br, CHF₂, F, or I;

X is O, S, or NR^{3A};

m is 0 or 1;

R⁴ is F or CF₃.

Each of the groups mentioned for a substituent in the tables is furthermore per se, independently of the combination in which it is mentioned, a particularly preferred aspect of the substituent in question.

According to particularly preferred embodiment of the compound of formula I, and their use as herbicide, compounds of the invention are the compounds of the formulae I-A to I-G that are compiled in the Tables 1 to 26, wherein the meaning for the combination of variables R¹, R², and R³ for each individual compound of tables 1 to 26 corresponds to each line of Table A.

The term “formula” used in below tables 1 to 26 denotes “compounds of formula”.

Table 1. Compounds of formula I.A, wherein m is 0 (=formula 1.1).

Table 2. Compounds of formula I.A, wherein m is 1, R⁴ is 4″-F (=formula 1.2).

Table 3. Compounds of formula I.B, wherein m is 0 (=formula 1.3).

Table 4. Compounds of formula I.B, wherein m is 1, R⁴ is 4″-F (=formula 1.4).

Table 5. Compounds of formula I.C, wherein m is 0 (=formula 1.5).

Table 6. Compounds of formula I.C, wherein m is 1, R⁴ is 2″-F (=formula 1.6).

Table 7. Compounds of formula I.D, wherein m is 0 (=formula 1.7).

Table 8. Compounds of formula I.D, wherein m is 1, R⁴ is 4″-F (=formula 1.8).

Table 9. Compounds of formula I.E, wherein m is 0, X is O (=formula 1.9).

Table 10. Compounds of formula I.E, wherein m is 1, X is O, R⁴ is 3″-F (=formula 1.10).

Table 11. Compounds of formula I.E, wherein m is 0, X is S(=formula 1.11).

Table 12. Compounds of formula I.E, wherein m is 1, X is S, R⁴ is 3″-F (=formula 1.12).

Table 13. Compounds of formula I.E, wherein m is 0, X is NCH₃ (=formula 1.13).

Table 14. Compounds of formula I.E, wherein m is 1, X is NCH₃, R⁴ is 3″-F (=formula 1.14).

Table 15. Compounds of formula I.F, wherein m is 0, X is O (=formula 1.15).

Table 16. Compounds of formula I.F, wherein m is 1, X is O, R⁴ is 5″-F (=formula 1.16).

Table 17. Compounds of formula I.F, wherein m is 0, X is S(=formula 1.17).

Table 18. Compounds of formula I.F, wherein m is 1, X is S, R⁴ is 5″-F (=formula 1.18).

Table 19. Compounds of formula I.F, wherein m is 0, X is NH (=formula 1.19).

Table 20. Compounds of formula I.F, wherein m is 1, X is NH, R⁴ is 5″-F (=formula 1.20).

Table 21. Compounds of formula I.G, wherein m is 0, X is (=formula 1.21).

Table 22. Compounds of formula I.G, wherein m is 1, X is 0, R⁴ is 5″-F (=formula 1.22).

Table 23. Compounds of formula I.G, wherein m is 0, X is S(=formula 1.23).

Table 24. Compounds of formula I.G, wherein m is 1, X is S, R⁴ is 5″-F (=formula 1.24).

Table 25. Compounds of formula I.G, wherein m is 0, X is NH (=formula 1.25).

Table 26. Compounds of formula I.G, wherein m is 1, X is NH, R⁴ is 5″-F (=formula 1.26).PG-31M

TABLE A

| Line | R¹ | R² | R³ |
|---|---|---|---|
| I-1 | c-C₃H₅ | CH═CH—CH₃ | CH₃ |
| I-2 | c-C₃H₅ | CH═CH—CH₃ | OCH₃ |
| I-3 | c-C₃H₅ | CH═CH—CH₃ | Cl |
| I-4 | c-C₃H₅ | CH═CH—CH₃ | Br |
| I-5 | c-C₃H₅ | CH═CH—CH₃ | CHF₂ |
| I-6 | c-C₃H₅ | CH═CH—CH₃ | F |
| I-7 | c-C₃H₅ | CH═CH—CH₃ | I |
| I-8 | c-C₃H₅ | CH═C(CH₂)₃ | CH₃ |
| I-9 | c-C₃H₅ | CH═C(CH₂)₃ | OCH₃ |
| I-10 | c-C₃H₅ | CH═C(CH₂)₃ | Cl |
| I-11 | c-C₃H₅ | CH═C(CH₂)₃ | Br |
| I-12 | c-C₃H₅ | CH═C(CH₂)₃ | CHF₂ |
| I-13 | c-C₃H₅ | CH═C(CH₂)₃ | F |
| I-14 | c-C₃H₅ | CH═C(CH₂)₃ | I |
| I-15 | c-C₃H₅ | CH═C(CH₂)₄ | CH₃ |
| I-16 | c-C₃H₅ | CH═C(CH₂)₄ | OCH₃ |
| I-17 | c-C₃H₅ | CH═C(CH₂)₄ | Cl |
| I-18 | c-C₃H₅ | CH═C(CH₂)₄ | Br |
| I-19 | c-C₃H₅ | CH═C(CH₂)₄ | CHF₂ |
| I-20 | c-C₃H₅ | CH═C(CH₂)₄ | F |
| I-21 | c-C₃H₅ | CH═C(CH₂)₄ | I |
| I-22 | c-C₃H₅ | 2-furyl | CH₃ |
| I-23 | c-C₃H₅ | 2-furyl | OCH₃ |
| I-24 | c-C₃H₅ | 2-furyl | Cl |
| I-25 | c-C₃H₅ | 2-furyl | Br |
| I-26 | c-C₃H₅ | 2-furyl | CHF₂ |
| I-27 | c-C₃H₅ | 2-furyl | F |
| I-28 | c-C₃H₅ | 2-furyl | I |
| I-29 | c-C₃H₅ | 3-furyl | CH₃ |
| I-30 | c-C₃H₅ | 3-furyl | OCH₃ |
| I-31 | c-C₃H₅ | 3-furyl | Cl |
| I-32 | c-C₃H₅ | 3-furyl | Br |
| I-33 | c-C₃H₅ | 3-furyl | CHF₂ |
| I-34 | c-C₃H₅ | 3-furyl | F |
| I-35 | c-C₃H₅ | 3-furyl | I |
| I-36 | c-C₃H₅ | 3-methyl-2-furyl | CH₃ |
| I-37 | c-C₃H₅ | 3-methyl-2-furyl | OCH₃ |
| I-38 | c-C₃H₅ | 3-methyl-2-furyl | Cl |
| I-39 | c-C₃H₅ | 3-methyl-2-furyl | Br |
| I-40 | c-C₃H₅ | 3-methyl-2-furyl | CHF₂ |
| I-41 | c-C₃H₅ | 3-methyl-2-furyl | F |
| I-42 | c-C₃H₅ | 3-methyl-2-furyl | I |
| I-43 | c-C₃H₅ | 2-methyl-3-furyl | CH₃ |
| I-44 | c-C₃H₅ | 2-methyl-3-furyl | OCH₃ |
| I-45 | c-C₃H₅ | 2-methyl-3-furyl | Cl |
| I-46 | c-C₃H₅ | 2-methyl-3-furyl | Br |
| I-47 | c-C₃H₅ | 2-methyl-3-furyl | CHF₂ |
| I-48 | c-C₃H₅ | 2-methyl-3-furyl | F |
| I-49 | c-C₃H₅ | 2-methyl-3-furyl | I |
| I-50 | c-C₃H₅ | CHOH—CHOH—C₆H₅ | CH₃ |
| I-51 | c-C₃H₅ | CHOH—CHOH—C₆H₅ | OCH₃ |
| I-52 | c-C₃H₅ | CHOH—CHOH—C₆H₅ | Cl |
| I-53 | c-C₃H₅ | CHOH—CHOH—C₆H₅ | Br |
| I-54 | c-C₃H₅ | CHOH—CHOH—C₆H₅ | CHF₂ |
| I-55 | c-C₃H₅ | CHOH—CHOH—C₆H₅ | F |
| I-56 | c-C₃H₅ | CHOH—CHOH—C₆H₅ | I |
| I-57 | c-C₃H₅ | CHOH—CHOH-2-furyl | CH₃ |
| I-58 | c-C₃H₅ | CHOH—CHOH-2-furyl | OCH₃ |
| I-59 | c-C₃H₅ | CHOH—CHOH-2-furyl | Cl |
| I-60 | c-C₃H₅ | CHOH—CHOH-2-furyl | Br |
| I-61 | c-C₃H₅ | CHOH—CHOH-2-furyl | CHF₂ |
| I-62 | c-C₃H₅ | CHOH—CHOH-2-furyl | F |
| I-63 | c-C₃H₅ | CHOH—CHOH-2-furyl | I |
| I-64 | c-C₃H₅ | 4-methyl-5-oxazolyl | CH₃ |
| I-65 | c-C₃H₅ | 4-methyl-5-oxazolyl | OCH₃ |
| I-66 | c-C₃H₅ | 4-methyl-5-oxazolyl | Cl |
| I-67 | c-C₃H₅ | 4-methyl-5-oxazolyl | Br |
| I-68 | c-C₃H₅ | 4-methyl-5-oxazolyl | CHF₂ |
| I-69 | c-C₃H₅ | 4-methyl-5-oxazolyl | F |
| I-70 | c-C₃H₅ | 4-methyl-5-oxazolyl | I |
| I-71 | c-C₄H₇ | CH═CH—CH₃ | CH₃ |
| I-72 | c-C₄H₇ | CH═CH—CH₃ | OCH₃ |
| I-73 | c-C₄H₇ | CH═CH—CH₃ | Cl |
| I-74 | c-C₄H₇ | CH═CH—CH₃ | Br |
| I-75 | c-C₄H₇ | CH═CH—CH₃ | CHF₂ |
| I-76 | c-C₄H₇ | CH═CH—CH₃ | F |
| I-77 | c-C₄H₇ | CH═CH—CH₃ | I |
| I-78 | c-C₄H₇ | CH═C(CH₂)₃ | CH₃ |
| I-79 | c-C₄H₇ | CH═C(CH₂)₃ | OCH₃ |
| I-80 | c-C₄H₇ | CH═C(CH₂)₃ | Cl |
| I-81 | c-C₄H₇ | CH═C(CH₂)₃ | Br |
| I-82 | c-C₄H₇ | CH═C(CH₂)₃ | CHF₂ |
| I-83 | c-C₄H₇ | CH═C(CH₂)₃ | F |
| I-84 | c-C₄H₇ | CH═C(CH₂)₃ | I |
| I-85 | c-C₄H₇ | CH═C(CH₂)₄ | CH₃ |
| I-86 | c-C₄H₇ | CH═C(CH₂)₄ | OCH₃ |
| I-87 | c-C₄H₇ | CH═C(CH₂)₄ | Cl |
| I-88 | c-C₄H₇ | CH═C(CH₂)₄ | Br |
| I-89 | c-C₄H₇ | CH═C(CH₂)₄ | CHF₂ |
| I-90 | c-C₄H₇ | CH═C(CH₂)₄ | F |
| I-91 | c-C₄H₇ | CH═C(CH₂)₄ | I |
| I-92 | c-C₄H₇ | 2-furyl | CH₃ |
| I-93 | c-C₄H₇ | 2-furyl | OCH₃ |
| I-94 | c-C₄H₇ | 2-furyl | Cl |
| I-95 | c-C₄H₇ | 2-furyl | Br |
| I-96 | c-C₄H₇ | 2-furyl | CHF₂ |
| I-97 | c-C₄H₇ | 2-furyl | F |
| I-98 | c-C₄H₇ | 2-furyl | I |
| I-99 | c-C₄H₇ | 3-furyl | CH₃ |
| I-100 | c-C₄H₇ | 3-furyl | OCH₃ |
| I-101 | c-C₄H₇ | 3-furyl | Cl |
| I-102 | c-C₄H₇ | 3-furyl | Br |
| I-103 | c-C₄H₇ | 3-furyl | CHF₂ |
| I-104 | c-C₄H₇ | 3-furyl | F |
| I-105 | c-C₄H₇ | 3-furyl | I |
| I-106 | c-C₄H₇ | 3-methyl-2-furyl | CH₃ |
| I-107 | c-C₄H₇ | 3-methyl-2-furyl | OCH₃ |
| I-108 | c-C₄H₇ | 3-methyl-2-furyl | Cl |
| I-109 | c-C₄H₇ | 3-methyl-2-furyl | Br |
| I-110 | c-C₄H₇ | 3-methyl-2-furyl | CHF₂ |
| I-111 | c-C₄H₇ | 3-methyl-2-furyl | F |
| I-112 | c-C₄H₇ | 3-methyl-2-furyl | I |
| I-113 | c-C₄H₇ | 2-methyl-3-furyl | CH₃ |
| I-114 | c-C₄H₇ | 2-methyl-3-furyl | OCH₃ |
| I-115 | c-C₄H₇ | 2-methyl-3-furyl | Cl |
| I-116 | c-C₄H₇ | 2-methyl-3-furyl | Br |
| I-117 | c-C₄H₇ | 2-methyl-3-furyl | CHF₂ |
| I-118 | c-C₄H₇ | 2-methyl-3-furyl | F |
| I-119 | c-C₄H₇ | 2-methyl-3-furyl | I |
| I-120 | c-C₄H₇ | CHOH—CHOH—C₆H₅ | CH₃ |
| I-121 | c-C₄H₇ | CHOH—CHOH—C₆H₅ | OCH₃ |
| I-122 | c-C₄H₇ | CHOH—CHOH—C₆H₅ | Cl |
| I-123 | c-C₄H₇ | CHOH—CHOH—C₆H₅ | Br |
| I-124 | c-C₄H₇ | CHOH—CHOH—C₆H₅ | CHF₂ |
| I-125 | c-C₄H₇ | CHOH—CHOH—C₆H₅ | F |
| I-126 | c-C₄H₇ | CHOH—CHOH—C₆H₅ | I |
| I-127 | c-C₄H₇ | CHOH—CHOH-2-furyl | CH₃ |
| I-128 | c-C₄H₇ | CHOH—CHOH-2-furyl | OCH₃ |
| I-129 | c-C₄H₇ | CHOH—CHOH-2-furyl | Cl |
| I-130 | c-C₄H₇ | CHOH—CHOH-2-furyl | Br |
| I-131 | c-C₄H₇ | CHOH—CHOH-2-furyl | CHF₂ |
| I-132 | c-C₄H₇ | CHOH—CHOH-2-furyl | F |
| I-133 | c-C₄H₇ | CHOH—CHOH-2-furyl | I |
| I-134 | c-C₄H₇ | 4-methyl-5-oxazolyl | CH₃ |
| I-135 | c-C₄H₇ | 4-methyl-5-oxazolyl | OCH₃ |
| I-136 | c-C₄H₇ | 4-methyl-5-oxazolyl | Cl |
| I-137 | c-C₄H₇ | 4-methyl-5-oxazolyl | Br |
| I-138 | c-C₄H₇ | 4-methyl-5-oxazolyl | CHF₂ |
| I-139 | c-C₄H₇ | 4-methyl-5-oxazolyl | F |
| I-140 | c-C₄H₇ | 4-methyl-5-oxazolyl | I |
| I-141 | C₂H₅ | CH═CH—CH₃ | CH₃ |
| I-142 | C₂H₅ | CH═CH—CH₃ | OCH₃ |
| I-143 | C₂H₅ | CH═CH—CH₃ | Cl |
| I-144 | C₂H₅ | CH═CH—CH₃ | Br |
| I-145 | C₂H₅ | CH═CH—CH₃ | CHF₂ |
| I-146 | C₂H₅ | CH═CH—CH₃ | F |
| I-147 | C₂H₅ | CH═CH—CH₃ | I |
| I-148 | C₂H₅ | CH═C(CH₂)₃ | CH₃ |
| I-149 | C₂H₅ | CH═C(CH₂)₃ | OCH₃ |
| I-150 | C₂H₅ | CH═C(CH₂)₃ | Cl |
| I-151 | C₂H₅ | CH═C(CH₂)₃ | Br |
| I-152 | C₂H₅ | CH═C(CH₂)₃ | CHF₂ |
| I-153 | C₂H₅ | CH═C(CH₂)₃ | F |
| I-154 | C₂H₅ | CH═C(CH₂)₃ | I |
| I-155 | C₂H₅ | CH═C(CH₂)₄ | CH₃ |
| I-156 | C₂H₅ | CH═C(CH₂)₄ | OCH₃ |
| I-157 | C₂H₅ | CH═C(CH₂)₄ | Cl |
| I-158 | C₂H₅ | CH═C(CH₂)₄ | Br |
| I-159 | C₂H₅ | CH═C(CH₂)₄ | CHF₂ |
| I-160 | C₂H₅ | CH═C(CH₂)₄ | F |
| I-161 | C₂H₅ | CH═C(CH₂)₄ | I |
| I-162 | C₂H₅ | 2-furyl | CH₃ |
| I-163 | C₂H₅ | 2-furyl | OCH₃ |
| I-164 | C₂H₅ | 2-furyl | Cl |
| I-165 | C₂H₅ | 2-furyl | Br |
| I-166 | C₂H₅ | 2-furyl | CHF₂ |
| I-167 | C₂H₅ | 2-furyl | F |
| I-168 | C₂H₅ | 2-furyl | I |
| I-169 | C₂H₅ | 3-furyl | CH₃ |
| I-170 | C₂H₅ | 3-furyl | OCH₃ |
| I-171 | C₂H₅ | 3-furyl | Cl |
| I-172 | C₂H₅ | 3-furyl | Br |
| I-173 | C₂H₅ | 3-furyl | CHF₂ |
| I-174 | C₂H₅ | 3-furyl | F |
| I-175 | C₂H₅ | 3-furyl | I |
| I-176 | C₂H₅ | 3-methyl-2-furyl | CH₃ |
| I-177 | C₂H₅ | 3-methyl-2-furyl | OCH₃ |
| I-178 | C₂H₅ | 3-methyl-2-furyl | Cl |
| I-179 | C₂H₅ | 3-methyl-2-furyl | Br |
| I-180 | C₂H₅ | 3-methyl-2-furyl | CHF₂ |
| I-181 | C₂H₅ | 3-methyl-2-furyl | F |
| I-182 | C₂H₅ | 3-methyl-2-furyl | I |
| I-183 | C₂H₅ | 2-methyl-3-furyl | CH₃ |
| I-184 | C₂H₅ | 2-methyl-3-furyl | OCH₃ |
| I-185 | C₂H₅ | 2-methyl-3-furyl | Cl |
| I-186 | C₂H₅ | 2-methyl-3-furyl | Br |
| I-187 | C₂H₅ | 2-methyl-3-furyl | CHF₂ |
| I-188 | C₂H₅ | 2-methyl-3-furyl | F |
| I-189 | C₂H₅ | 2-methyl-3-furyl | I |
| I-190 | C₂H₅ | CHOH—CHOH—C₆H₅ | CH₃ |
| I-191 | C₂H₅ | CHOH—CHOH—C₆H₅ | OCH₃ |
| I-192 | C₂H₅ | CHOH—CHOH—C₆H₅ | Cl |
| I-193 | C₂H₅ | CHOH—CHOH—C₆H₅ | Br |
| I-194 | C₂H₅ | CHOH—CHOH—C₆H₅ | CHF₂ |
| I-195 | C₂H₅ | CHOH—CHOH—C₆H₅ | F |
| I-196 | C₂H₅ | CHOH—CHOH—C₆H₅ | I |
| I-197 | C₂H₅ | CHOH—CHOH-2-furyl | CH₃ |
| I-198 | C₂H₅ | CHOH—CHOH-2-furyl | OCH₃ |
| I-199 | C₂H₅ | CHOH—CHOH-2-furyl | Cl |
| I-200 | C₂H₅ | CHOH—CHOH-2-furyl | Br |
| I-201 | C₂H₅ | CHOH—CHOH-2-furyl | CHF₂ |
| I-202 | C₂H₅ | CHOH—CHOH-2-furyl | F |
| I-203 | C₂H₅ | CHOH—CHOH-2-furyl | I |
| I-204 | C₂H₅ | 4-methyl-5-oxazolyl | CH₃ |
| I-205 | C₂H₅ | 4-methyl-5-oxazolyl | OCH₃ |
| I-206 | C₂H₅ | 4-methyl-5-oxazolyl | Cl |
| I-207 | C₂H₅ | 4-methyl-5-oxazolyl | Br |
| I-208 | C₂H₅ | 4-methyl-5-oxazolyl | CHF₂ |
| I-209 | C₂H₅ | 4-methyl-5-oxazolyl | F |
| I-210 | C₂H₅ | 4-methyl-5-oxazolyl | I |
| I-211 | OCH₃ | CH═CH—CH₃ | CH₃ |
| I-212 | OCH₃ | CH═CH—CH₃ | OCH₃ |
| I-213 | OCH₃ | CH═CH—CH₃ | Cl |
| I-214 | OCH₃ | CH═CH—CH₃ | Br |
| I-215 | OCH₃ | CH═CH—CH₃ | CHF₂ |
| I-216 | OCH₃ | CH═CH—CH₃ | F |
| I-217 | OCH₃ | CH═CH—CH₃ | I |
| I-218 | OCH₃ | CH═C(CH₂)₃ | CH₃ |
| I-219 | OCH₃ | CH═C(CH₂)₃ | OCH₃ |
| I-220 | OCH₃ | CH═C(CH₂)₃ | Cl |
| I-221 | OCH₃ | CH═C(CH₂)₃ | Br |
| I-222 | OCH₃ | CH═C(CH₂)₃ | CHF₂ |
| I-223 | OCH₃ | CH═C(CH₂)₃ | F |
| I-224 | OCH₃ | CH═C(CH₂)₃ | I |
| I-225 | OCH₃ | CH═C(CH₂)₄ | CH₃ |
| I-226 | OCH₃ | CH═C(CH₂)₄ | OCH₃ |
| I-227 | OCH₃ | CH═C(CH₂)₄ | Cl |
| I-228 | OCH₃ | CH═C(CH₂)₄ | Br |
| I-229 | OCH₃ | CH═C(CH₂)₄ | CHF₂ |
| I-230 | OCH₃ | CH═C(CH₂)₄ | F |
| I-231 | OCH₃ | CH═C(CH₂)₄ | I |
| I-232 | OCH₃ | 2-furyl | CH₃ |
| I-233 | OCH₃ | 2-furyl | OCH₃ |
| I-234 | OCH₃ | 2-furyl | Cl |
| I-235 | OCH₃ | 2-furyl | Br |
| I-236 | OCH₃ | 2-furyl | CHF₂ |
| I-237 | OCH₃ | 2-furyl | F |
| I-238 | OCH₃ | 2-furyl | I |
| I-239 | OCH₃ | 3-furyl | CH₃ |
| I-240 | OCH₃ | 3-furyl | OCH₃ |
| I-241 | OCH₃ | 3-furyl | Cl |
| I-242 | OCH₃ | 3-furyl | Br |
| I-243 | OCH₃ | 3-furyl | CHF₂ |
| I-244 | OCH₃ | 3-furyl | F |
| I-245 | OCH₃ | 3-furyl | I |
| I-246 | OCH₃ | 3-methyl-2-furyl | CH₃ |
| I-247 | OCH₃ | 3-methyl-2-furyl | OCH₃ |
| I-248 | OCH₃ | 3-methyl-2-furyl | Cl |
| I-249 | OCH₃ | 3-methyl-2-furyl | Br |
| I-250 | OCH₃ | 3-methyl-2-furyl | CHF₂ |
| I-251 | OCH₃ | 3-methyl-2-furyl | F |
| I-252 | OCH₃ | 3-methyl-2-furyl | I |
| I-253 | OCH₃ | 2-methyl-3-furyl | CH₃ |
| I-254 | OCH₃ | 2-methyl-3-furyl | OCH₃ |
| I-255 | OCH₃ | 2-methyl-3-furyl | Cl |
| I-256 | OCH₃ | 2-methyl-3-furyl | Br |
| I-257 | OCH₃ | 2-methyl-3-furyl | CHF₂ |
| I-258 | OCH₃ | 2-methyl-3-furyl | F |
| I-259 | OCH₃ | 2-methyl-3-furyl | I |
| I-260 | OCH₃ | CHOH—CHOH—C₆H₅ | CH₃ |
| I-261 | OCH₃ | CHOH—CHOH—C₆H₅ | OCH₃ |
| I-262 | OCH₃ | CHOH—CHOH—C₆H₅ | Cl |
| I-263 | OCH₃ | CHOH—CHOH—C₆H₅ | Br |
| I-264 | OCH₃ | CHOH—CHOH—C₆H₅ | CHF₂ |
| I-265 | OCH₃ | CHOH—CHOH—C₆H₅ | F |
| I-266 | OCH₃ | CHOH—CHOH—C₆H₅ | I |
| I-267 | OCH₃ | CHOH—CHOH-2-furyl | CH₃ |
| I-268 | OCH₃ | CHOH—CHOH-2-furyl | OCH₃ |
| I-269 | OCH₃ | CHOH—CHOH-2-furyl | Cl |
| I-270 | OCH₃ | CHOH—CHOH-2-furyl | Br |
| I-271 | OCH₃ | CHOH—CHOH-2-furyl | CHF₂ |
| I-272 | OCH₃ | CHOH—CHOH-2-furyl | F |
| I-273 | OCH₃ | CHOH—CHOH-2-furyl | I |
| I-274 | OCH₃ | 4-methyl-5-oxazolyl | CH₃ |
| I-275 | OCH₃ | 4-methyl-5-oxazolyl | OCH₃ |
| I-276 | OCH₃ | 4-methyl-5-oxazolyl | Cl |
| I-277 | OCH₃ | 4-methyl-5-oxazolyl | Br |
| I-278 | OCH₃ | 4-methyl-5-oxazolyl | CHF₂ |
| I-279 | OCH₃ | 4-methyl-5-oxazolyl | F |
| I-280 | OCH₃ | 4-methyl-5-oxazolyl | I |

The specific number for each single compound is deductible as follows:

Compound 1.1.I-3 e.g. comprises the compound of formula 1.1 from Table 1 and line I-3 from Table A;

According to another particularly preferred embodiment of the compounds of formula I and their use as herbicide, compounds of the invention are the compounds of formulae I.A or I.C, wherein

R¹ is C₂H₅, c-C₃H₅, c-C₄H₇, or OCH₃;

R² is selected from R²-9.1 to R²-9.676 from Table R²-9, R²-10.1 to R²-10.676 from Table R²-10, and R²-15.1 to R²-15.676 from Table R²-15;

R³ is CH₃, OCH₃, Cl, Br, CHF₂, F, or I;

m is 0 or 1;

R⁴ is F.

According to another particularly preferred embodiment of the compounds of formula I and their use as herbicide, compounds of the invention are the compounds of formulae I.A that are compiled in tables 27 to 2054, wherein the meaning for the combination of variables R¹, R³, m, and R⁴ for each individual compound of tables 27 to 2054 corresponds to each line of Table A1. Each of the groups mentioned for a substituent in the tables is furthermore per se, independently of the combination in which it is mentioned, a particularly preferred aspect of the substituent in question.

The term “formula” used in below tables 27 to 2054 denotes “compounds of formula”.

Table 27. Compounds of formula I.A, wherein R² is R²-9.17(=formula 1.27).

Table 28. Compounds of formula I.A, wherein R² is R²-9.18(=formula 1.28).

Table 29. Compounds of formula I.A, wherein R² is R²-9.19(=formula 1.29).

Table 30. Compounds of formula I.A, wherein R² is R²-9.20(=formula 1.30).

Table 31. Compounds of formula I.A, wherein R² is R²-9.21(=formula 1.31).

Table 32. Compounds of formula I.A, wherein R² is R²-9.22(=formula 1.32).

Table 33. Compounds of formula I.A, wherein R² is R²-9.23(=formula 1.33).

Table 34. Compounds of formula I.A, wherein R² is R²-9.24(=formula 1.34).

Table 35. Compounds of formula I.A, wherein R² is R²-9.25(=formula 1.35).

Table 36. Compounds of formula I.A, wherein R² is R²-9.26(=formula 1.36).

Table 37. Compounds of formula I.A, wherein R² is R²-9.27(=formula 1.37).

Table 38. Compounds of formula I.A, wherein R² is R²-9.28(=formula 1.38).

Table 39. Compounds of formula I.A, wherein R² is R²-9.29(=formula 1.39).

Table 40. Compounds of formula I.A, wherein R² is R²-9.30(=formula 1.40).

Table 41. Compounds of formula I.A, wherein R² is R²-9.31(=formula 1.41).

Table 42. Compounds of formula I.A, wherein R² is R²-9.32(=formula 1.42).

Table 43. Compounds of formula I.A, wherein R² is R²-9.33(=formula 1.43).

Table 44. Compounds of formula I.A, wherein R² is R²-9.34(=formula 1.44).

Table 45. Compounds of formula I.A, wherein R² is R²-9.35(=formula 1.45).

Table 46. Compounds of formula I.A, wherein R² is R²-9.36(=formula 1.46).

Table 47. Compounds of formula I.A, wherein R² is R²-9.37(=formula 1.47).

Table 48. Compounds of formula I.A, wherein R² is R²-9.38(=formula 1.48).

Table 49. Compounds of formula I.A, wherein R² is R²-9.39(=formula 1.49).

Table 50. Compounds of formula I.A, wherein R² is R²-9.40(=formula 1.50).

Table 51. Compounds of formula I.A, wherein R² is R²-9.41(=formula 1.51).

Table 52. Compounds of formula I.A, wherein R² is R²-9.42(=formula 1.52).

Table 53. Compounds of formula I.A, wherein R² is R²-9.43(=formula 1.53).

Table 54. Compounds of formula I.A, wherein R² is R²-9.44(=formula 1.54).

Table 55. Compounds of formula I.A, wherein R² is R²-9.45(=formula 1.55).

Table 56. Compounds of formula I.A, wherein R² is R²-9.46(=formula 1.56).

Table 57. Compounds of formula I.A, wherein R² is R²-9.47(=formula 1.57).

Table 58. Compounds of formula I.A, wherein R² is R²-9.48(=formula 1.58).

Table 59. Compounds of formula I.A, wherein R² is R²-9.49(=formula 1.59).

Table 60. Compounds of formula I.A, wherein R² is R²-9.50(=formula 1.60).

Table 61. Compounds of formula I.A, wherein R² is R²-9.51(=formula 1.61).

Table 62. Compounds of formula I.A, wherein R² is R²-9.52(=formula 1.62).

Table 63. Compounds of formula I.A, wherein R² is R²-9.53(=formula 1.63).

Table 64. Compounds of formula I.A, wherein R² is R²-9.54(=formula 1.64).

Table 65. Compounds of formula I.A, wherein R² is R²-9.55(=formula 1.65).

Table 66. Compounds of formula I.A, wherein R² is R²-9.56(=formula 1.66).

Table 67. Compounds of formula I.A, wherein R² is R²-9.57(=formula 1.67).

Table 68. Compounds of formula I.A, wherein R² is R²-9.58(=formula 1.68).

Table 69. Compounds of formula I.A, wherein R² is R²-9.59(=formula 1.69).

Table 70. Compounds of formula I.A, wherein R² is R²-9.60(=formula 1.70).

Table 71. Compounds of formula I.A, wherein R² is R²-9.61(=formula 1.71).

Table 72. Compounds of formula I.A, wherein R² is R²-9.62(=formula 1.72).

Table 73. Compounds of formula I.A, wherein R² is R²-9.63(=formula 1.73).

Table 74. Compounds of formula I.A, wherein R² is R²-9.64(=formula 1.74).

Table 75. Compounds of formula I.A, wherein R² is R²-9.65(=formula 1.75).

Table 76. Compounds of formula I.A, wherein R² is R²-9.66(=formula 1.76).

Table 77. Compounds of formula I.A, wherein R² is R²-9.67(=formula 1.77).

Table 78. Compounds of formula I.A, wherein R² is R²-9.68(=formula 1.78).

Table 79. Compounds of formula I.A, wherein R² is R²-9.69(=formula 1.79).

Table 80. Compounds of formula I.A, wherein R² is R²-9.70(=formula 1.80).

Table 81. Compounds of formula I.A, wherein R² is R²-9.71(=formula 1.81).

Table 82. Compounds of formula I.A, wherein R² is R²-9.72(=formula 1.82).

Table 83. Compounds of formula I.A, wherein R² is R²-9.73(=formula 1.83).

Table 84. Compounds of formula I.A, wherein R² is R²-9.74(=formula 1.84).

Table 85. Compounds of formula I.A, wherein R² is R²-9.75(=formula 1.85).

Table 86. Compounds of formula I.A, wherein R² is R²-9.76(=formula 1.86).

Table 87. Compounds of formula I.A, wherein R² is R²-9.77(=formula 1.87).

Table 88. Compounds of formula I.A, wherein R² is R²-9.78(=formula 1.88).

Table 89. Compounds of formula I.A, wherein R² is R²-9.79(=formula 1.89).

Table 90. Compounds of formula I.A, wherein R² is R²-9.80(=formula 1.90).

Table 91. Compounds of formula I.A, wherein R² is R²-9.81(=formula 1.91).

Table 92. Compounds of formula I.A, wherein R² is R²-9.82(=formula 1.92).

Table 93. Compounds of formula I.A, wherein R² is R²-9.83(=formula 1.93).

Table 94. Compounds of formula I.A, wherein R² is R²-9.84(=formula 1.94).

Table 95. Compounds of formula I.A, wherein R² is R²-9.85(=formula 1.95).

Table 96. Compounds of formula I.A, wherein R² is R²-9.86(=formula 1.96).

Table 97. Compounds of formula I.A, wherein R² is R²-9.87(=formula 1.97).

Table 98. Compounds of formula I.A, wherein R² is R²-9.88(=formula 1.98).

Table 99. Compounds of formula I.A, wherein R² is R²-9.89(=formula 1.99).

Table 100. Compounds of formula I.A, wherein R² is R²-9.90(=formula 1.100).

Table 101. Compounds of formula I.A, wherein R² is R²-9.91(=formula 1.101).

Table 102. Compounds of formula I.A, wherein R² is R²-9.92(=formula 1.102).

Table 103. Compounds of formula I.A, wherein R² is R²-9.93(=formula 1.103).

Table 104. Compounds of formula I.A, wherein R² is R²-9.94(=formula 1.104).

Table 105. Compounds of formula I.A, wherein R² is R²-9.95(=formula 1.105).

Table 106. Compounds of formula I.A, wherein R² is R²-9.96(=formula 1.106).

Table 107. Compounds of formula I.A, wherein R² is R²-9.97(=formula 1.107).

Table 108. Compounds of formula I.A, wherein R² is R²-9.98(=formula 1.108).

Table 109. Compounds of formula I.A, wherein R² is R²-9.99(=formula 1.109).

Table 110. Compounds of formula I.A, wherein R² is R²-9.100(=formula 1.110).

Table 111. Compounds of formula I.A, wherein R² is R²-9.101(=formula 1.111).

Table 112. Compounds of formula I.A, wherein R² is R²-9.102(=formula 1.112).

Table 113. Compounds of formula I.A, wherein R² is R²-9.103(=formula 1.113).

Table 114. Compounds of formula I.A, wherein R² is R²-9.104(=formula 1.114).

Table 115. Compounds of formula I.A, wherein R² is R²-9.105(=formula 1.115).

Table 116. Compounds of formula I.A, wherein R² is R²-9.106(=formula 1.116).

Table 117. Compounds of formula I.A, wherein R² is R²-9.107(=formula 1.117).

Table 118. Compounds of formula I.A, wherein R² is R²-9.108(=formula 1.118).

Table 119. Compounds of formula I.A, wherein R² is R²-9.109(=formula 1.119).

Table 120. Compounds of formula I.A, wherein R² is R²-9.110(=formula 1.120).

Table 121. Compounds of formula I.A, wherein R² is R²-9.111(=formula 1.121).

Table 122. Compounds of formula I.A, wherein R² is R²-9.112(=formula 1.122).

Table 123. Compounds of formula I.A, wherein R² is R²-9.113(=formula 1.123).

Table 124. Compounds of formula I.A, wherein R² is R²-9.114(=formula 1.124).

Table 125. Compounds of formula I.A, wherein R² is R²-9.115(=formula 1.125).

Table 126. Compounds of formula I.A, wherein R² is R²-9.116(=formula 1.126).

Table 127. Compounds of formula I.A, wherein R² is R²-9.117(=formula 1.127).

Table 128. Compounds of formula I.A, wherein R² is R²-9.118(=formula 1.128).

Table 129. Compounds of formula I.A, wherein R² is R²-9.119(=formula 1.129).

Table 130. Compounds of formula I.A, wherein R² is R²-9.120(=formula 1.130).

Table 131. Compounds of formula I.A, wherein R² is R²-9.121(=formula 1.131).

Table 132. Compounds of formula I.A, wherein R² is R²-9.122(=formula 1.132).

Table 133. Compounds of formula I.A, wherein R² is R²-9.123(=formula 1.133).

Table 134. Compounds of formula I.A, wherein R² is R²-9.124(=formula 1.134).

Table 135. Compounds of formula I.A, wherein R² is R²-9.125(=formula 1.135).

Table 136. Compounds of formula I.A, wherein R² is R²-9.126(=formula 1.136).

Table 137. Compounds of formula I.A, wherein R² is R²-9.127(=formula 1.137).

Table 138. Compounds of formula I.A, wherein R² is R²-9.128(=formula 1.138).

Table 139. Compounds of formula I.A, wherein R² is R²-9.129(=formula 1.139).

Table 140. Compounds of formula I.A, wherein R² is R²-9.130(=formula 1.140).

Table 141. Compounds of formula I.A, wherein R² is R²-9.131(=formula 1.141).

Table 142. Compounds of formula I.A, wherein R² is R²-9.132(=formula 1.142).

Table 143. Compounds of formula I.A, wherein R² is R²-9.133(=formula 1.143).

Table 144. Compounds of formula I.A, wherein R² is R²-9.134(=formula 1.144).

Table 145. Compounds of formula I.A, wherein R² is R²-9.135(=formula 1.145).

Table 146. Compounds of formula I.A, wherein R² is R²-9.136(=formula 1.146).

Table 147. Compounds of formula I.A, wherein R² is R²-9.137(=formula 1.147).

Table 148. Compounds of formula I.A, wherein R² is R²-9.138(=formula 1.148).

Table 149. Compounds of formula I.A, wherein R² is R²-9.139(=formula 1.149).

Table 150. Compounds of formula I.A, wherein R² is R²-9.140(=formula 1.150).

Table 151. Compounds of formula I.A, wherein R² is R²-9.141(=formula 1.151).

Table 152. Compounds of formula I.A, wherein R² is R²-9.142(=formula 1.152).

Table 153. Compounds of formula I.A, wherein R² is R²-9.143(=formula 1.153).

Table 154. Compounds of formula I.A, wherein R² is R²-9.144(=formula 1.154).

Table 155. Compounds of formula I.A, wherein R² is R²-9.145(=formula 1.155).

Table 156. Compounds of formula I.A, wherein R² is R²-9.146(=formula 1.156).

Table 157. Compounds of formula I.A, wherein R² is R²-9.147(=formula 1.157).

Table 158. Compounds of formula I.A, wherein R² is R²-9.148(=formula 1.158).

Table 159. Compounds of formula I.A, wherein R² is R²-9.149(=formula 1.159).

Table 160. Compounds of formula I.A, wherein R² is R²-9.150(=formula 1.160).

Table 161. Compounds of formula I.A, wherein R² is R²-9.151(=formula 1.161).

Table 162. Compounds of formula I.A, wherein R² is R²-9.152(=formula 1.162).

Table 163. Compounds of formula I.A, wherein R² is R²-9.153(=formula 1.163).

Table 164. Compounds of formula I.A, wherein R² is R²-9.154(=formula 1.164).

Table 165. Compounds of formula I.A, wherein R² is R²-9.155(=formula 1.165).

Table 166. Compounds of formula I.A, wherein R² is R²-9.156(=formula 1.166).

Table 167. Compounds of formula I.A, wherein R² is R²-9.157(=formula 1.167).

Table 168. Compounds of formula I.A, wherein R² is R²-9.158(=formula 1.168).

Table 169. Compounds of formula I.A, wherein R² is R²-9.159(=formula 1.169).

Table 170. Compounds of formula I.A, wherein R² is R²-9.160(=formula 1.170).

Table 171. Compounds of formula I.A, wherein R² is R²-9.161(=formula 1.171).

Table 172. Compounds of formula I.A, wherein R² is R²-9.162(=formula 1.172).

Table 173. Compounds of formula I.A, wherein R² is R²-9.163(=formula 1.173).

Table 174. Compounds of formula I.A, wherein R² is R²-9.164(=formula 1.174).

Table 175. Compounds of formula I.A, wherein R² is R²-9.165(=formula 1.175).

Table 176. Compounds of formula I.A, wherein R² is R²-9.166(=formula 1.176).

Table 177. Compounds of formula I.A, wherein R² is R²-9.167(=formula 1.177).

Table 178. Compounds of formula I.A, wherein R² is R²-9.168(=formula 1.178).

Table 179. Compounds of formula I.A, wherein R² is R²-9.169(=formula 1.179).

Table 180. Compounds of formula I.A, wherein R² is R²-9.170(=formula 1.180).

Table 181. Compounds of formula I.A, wherein R² is R²-9.171(=formula 1.181).

Table 182. Compounds of formula I.A, wherein R² is R²-9.172(=formula 1.182).

Table 183. Compounds of formula I.A, wherein R² is R²-9.173(=formula 1.183).

Table 184. Compounds of formula I.A, wherein R² is R²-9.174(=formula 1.184).

Table 185. Compounds of formula I.A, wherein R² is R²-9.175(=formula 1.185).

Table 186. Compounds of formula I.A, wherein R² is R²-9.176(=formula 1.186).

Table 187. Compounds of formula I.A, wherein R² is R²-9.177(=formula 1.187).

Table 188. Compounds of formula I.A, wherein R² is R²-9.178(=formula 1.188).

Table 189. Compounds of formula I.A, wherein R² is R²-9.179(=formula 1.189).

Table 190. Compounds of formula I.A, wherein R² is R²-9.180(=formula 1.190).

Table 191. Compounds of formula I.A, wherein R² is R²-9.181(=formula 1.191).

Table 192. Compounds of formula I.A, wherein R² is R²-9.182(=formula 1.192).

Table 193. Compounds of formula I.A, wherein R² is R²-9.183(=formula 1.193).

Table 194. Compounds of formula I.A, wherein R² is R²-9.184(=formula 1.194).

Table 195. Compounds of formula I.A, wherein R² is R²-9.185(=formula 1.195).

Table 196. Compounds of formula I.A, wherein R² is R²-9.186(=formula 1.196).

Table 197. Compounds of formula I.A, wherein R² is R²-9.187(=formula 1.197).

Table 198. Compounds of formula I.A, wherein R² is R²-9.188(=formula 1.198).

Table 199. Compounds of formula I.A, wherein R² is R²-9.189(=formula 1.199).

Table 200. Compounds of formula I.A, wherein R² is R²-9.190(=formula 1.200).

Table 201. Compounds of formula I.A, wherein R² is R²-9.191(=formula 1.201).

Table 202. Compounds of formula I.A, wherein R² is R²-9.192(=formula 1.202).

Table 203. Compounds of formula I.A, wherein R² is R²-9.193(=formula 1.203).

Table 204. Compounds of formula I.A, wherein R² is R²-9.194(=formula 1.204).

Table 205. Compounds of formula I.A, wherein R² is R²-9.195(=formula 1.205).

Table 206. Compounds of formula I.A, wherein R² is R²-9.196(=formula 1.206).

Table 207. Compounds of formula I.A, wherein R² is R²-9.197(=formula 1.207).

Table 208. Compounds of formula I.A, wherein R² is R²-9.198(=formula 1.208).

Table 209. Compounds of formula I.A, wherein R² is R²-9.199(=formula 1.209).

Table 210. Compounds of formula I.A, wherein R² is R²-9.200(=formula 1.210).

Table 211. Compounds of formula I.A, wherein R² is R²-9.201(=formula 1.211).

Table 212. Compounds of formula I.A, wherein R² is R²-9.202(=formula 1.212).

Table 213. Compounds of formula I.A, wherein R² is R²-9.203(=formula 1.213).

Table 214. Compounds of formula I.A, wherein R² is R²-9.204(=formula 1.214).

Table 215. Compounds of formula I.A, wherein R² is R²-9.205(=formula 1.215).

Table 216. Compounds of formula I.A, wherein R² is R²-9.206(=formula 1.216).

Table 217. Compounds of formula I.A, wherein R² is R²-9.207(=formula 1.217).

Table 218. Compounds of formula I.A, wherein R² is R²-9.208(=formula 1.218).

Table 219. Compounds of formula I.A, wherein R² is R²-9.209(=formula 1.219).

Table 220. Compounds of formula I.A, wherein R² is R²-9.210(=formula 1.220).

Table 221. Compounds of formula I.A, wherein R² is R²-9.211(=formula 1.221).

Table 222. Compounds of formula I.A, wherein R² is R²-9.212(=formula 1.222).

Table 223. Compounds of formula I.A, wherein R² is R²-9.213(=formula 1.223).

Table 224. Compounds of formula I.A, wherein R² is R²-9.214(=formula 1.224).

Table 225. Compounds of formula I.A, wherein R² is R²-9.215(=formula 1.225).

Table 226. Compounds of formula I.A, wherein R² is R²-9.216(=formula 1.226).

Table 227. Compounds of formula I.A, wherein R² is R²-9.217(=formula 1.227).

Table 228. Compounds of formula I.A, wherein R² is R²-9.218(=formula 1.228).

Table 229. Compounds of formula I.A, wherein R² is R²-9.219(=formula 1.229).

Table 230. Compounds of formula I.A, wherein R² is R²-9.220(=formula 1.230).

Table 231. Compounds of formula I.A, wherein R² is R²-9.221(=formula 1.231).

Table 232. Compounds of formula I.A, wherein R² is R²-9.222(=formula 1.232).

Table 233. Compounds of formula I.A, wherein R² is R²-9.223(=formula 1.233).

Table 234. Compounds of formula I.A, wherein R² is R²-9.224(=formula 1.234).

Table 235. Compounds of formula I.A, wherein R² is R²-9.225(=formula 1.235).

Table 236. Compounds of formula I.A, wherein R² is R²-9.226(=formula 1.236).

Table 237. Compounds of formula I.A, wherein R² is R²-9.227(=formula 1.237).

Table 238. Compounds of formula I.A, wherein R² is R²-9.228(=formula 1.238).

Table 239. Compounds of formula I.A, wherein R² is R²-9.229(=formula 1.239).

Table 240. Compounds of formula I.A, wherein R² is R²-9.230(=formula 1.240).

Table 241. Compounds of formula I.A, wherein R² is R²-9.231(=formula 1.241).

Table 242. Compounds of formula I.A, wherein R² is R²-9.232(=formula 1.242).

Table 243. Compounds of formula I.A, wherein R² is R²-9.233(=formula 1.243).

Table 244. Compounds of formula I.A, wherein R² is R²-9.234(=formula 1.244).

Table 245. Compounds of formula I.A, wherein R² is R²-9.235(=formula 1.245).

Table 246. Compounds of formula I.A, wherein R² is R²-9.236(=formula 1.246).

Table 247. Compounds of formula I.A, wherein R² is R²-9.237(=formula 1.247).

Table 248. Compounds of formula I.A, wherein R² is R²-9.238(=formula 1.248).

Table 249. Compounds of formula I.A, wherein R² is R²-9.239(=formula 1.249).

Table 250. Compounds of formula I.A, wherein R² is R²-9.240(=formula 1.250).

Table 251. Compounds of formula I.A, wherein R² is R²-9.241(=formula 1.251).

Table 252. Compounds of formula I.A, wherein R² is R²-9.242(=formula 1.252).

Table 253. Compounds of formula I.A, wherein R² is R²-9.243 (=formula 1.253).

Table 254. Compounds of formula I.A, wherein R² is R²-9.244(=formula 1.254).

Table 255. Compounds of formula I.A, wherein R² is R²-9.245(=formula 1.255).

Table 256. Compounds of formula I.A, wherein R² is R²-9.246(=formula 1.256).

Table 257. Compounds of formula I.A, wherein R² is R²-9.247(=formula 1.257).

Table 258. Compounds of formula I.A, wherein R² is R²-9.248(=formula 1.258).

Table 259. Compounds of formula I.A, wherein R² is R²-9.249(=formula 1.259).

Table 260. Compounds of formula I.A, wherein R² is R²-9.250(=formula 1.260).

Table 261. Compounds of formula I.A, wherein R² is R²-9.251(=formula 1.261).

Table 262. Compounds of formula I.A, wherein R² is R²-9.252(=formula 1.262).

Table 263. Compounds of formula I.A, wherein R² is R²-9.253(=formula 1.263).

Table 264. Compounds of formula I.A, wherein R² is R²-9.254(=formula 1.264).

Table 265. Compounds of formula I.A, wherein R² is R²-9.255(=formula 1.265).

Table 266. Compounds of formula I.A, wherein R² is R²-9.256(=formula 1.266).

Table 267. Compounds of formula I.A, wherein R² is R²-9.257(=formula 1.267).

Table 268. Compounds of formula I.A, wherein R² is R²-9.258(=formula 1.268).

Table 269. Compounds of formula I.A, wherein R² is R²-9.259(=formula 1.269).

Table 270. Compounds of formula I.A, wherein R² is R²-9.260(=formula 1.270).

Table 271. Compounds of formula I.A, wherein R² is R²-9.261(=formula 1.271).

Table 272. Compounds of formula I.A, wherein R² is R²-9.262(=formula 1.272).

Table 273. Compounds of formula I.A, wherein R² is R²-9.263(=formula 1.273).

Table 274. Compounds of formula I.A, wherein R² is R²-9.264(=formula 1.274).

Table 275. Compounds of formula I.A, wherein R² is R²-9.265 (=formula 1.275).

Table 276. Compounds of formula I.A, wherein R² is R²-9.266 (=formula 1.276).

Table 277. Compounds of formula I.A, wherein R² is R²-9.267(=formula 1.277).

Table 278. Compounds of formula I.A, wherein R² is R²-9.268(=formula 1.278).

Table 279. Compounds of formula I.A, wherein R² is R²-9.269(=formula 1.279).

Table 280. Compounds of formula I.A, wherein R² is R²-9.270 (=formula 1.280).

Table 281. Compounds of formula I.A, wherein R² is R²-9.271(=formula 1.281).

Table 282. Compounds of formula I.A, wherein R² is R²-9.272(=formula 1.282).

Table 283. Compounds of formula I.A, wherein R² is R²-9.273 (=formula 1.283).

Table 284. Compounds of formula I.A, wherein R² is R²-9.274 (=formula 1.284).

Table 285. Compounds of formula I.A, wherein R² is R²-9.275 (=formula 1.285).

Table 286. Compounds of formula I.A, wherein R² is R²-9.276 (=formula 1.286).

Table 287. Compounds of formula I.A, wherein R² is R²-9.277(=formula 1.287).

Table 288. Compounds of formula I.A, wherein R² is R²-9.278(=formula 1.288).

Table 289. Compounds of formula I.A, wherein R² is R²-9.279(=formula 1.289).

Table 290. Compounds of formula I.A, wherein R² is R²-9.280(=formula 1.290).

Table 291. Compounds of formula I.A, wherein R² is R²-9.281(=formula 1.291).

Table 292. Compounds of formula I.A, wherein R² is R²-9.282(=formula 1.292).

Table 293. Compounds of formula I.A, wherein R² is R²-9.283(=formula 1.293).

Table 294. Compounds of formula I.A, wherein R² is R²-9.284(=formula 1.294).

Table 295. Compounds of formula I.A, wherein R² is R²-9.285(=formula 1.295).

Table 296. Compounds of formula I.A, wherein R² is R²-9.286(=formula 1.296).

Table 297. Compounds of formula I.A, wherein R² is R²-9.287(=formula 1.297).

Table 298. Compounds of formula I.A, wherein R² is R²-9.288(=formula 1.298).

Table 299. Compounds of formula I.A, wherein R² is R²-9.289(=formula 1.299).

Table 300. Compounds of formula I.A, wherein R² is R²-9.290(=formula 1.300).

Table 301. Compounds of formula I.A, wherein R² is R²-9.291(=formula 1.301).

Table 302. Compounds of formula I.A, wherein R² is R²-9.292(=formula 1.302).

Table 303. Compounds of formula I.A, wherein R² is R²-9.293(=formula 1.303).

Table 304. Compounds of formula I.A, wherein R² is R²-9.294(=formula 1.304).

Table 305. Compounds of formula I.A, wherein R² is R²-9.295(=formula 1.305).

Table 306. Compounds of formula I.A, wherein R² is R²-9.296(=formula 1.306).

Table 307. Compounds of formula I.A, wherein R² is R²-9.297(=formula 1.307).

Table 308. Compounds of formula I.A, wherein R² is R²-9.298(=formula 1.308).

Table 309. Compounds of formula I.A, wherein R² is R²-9.299(=formula 1.309).

Table 310. Compounds of formula I.A, wherein R² is R²-9.300 (=formula 1.310).

Table 311. Compounds of formula I.A, wherein R² is R²-9.301(=formula 1.311).

Table 312. Compounds of formula I.A, wherein R² is R²-9.302(=formula 1.312).

Table 313. Compounds of formula I.A, wherein R² is R²-9.303(=formula 1.313).

Table 314. Compounds of formula I.A, wherein R² is R²-9.304(=formula 1.314).

Table 315. Compounds of formula I.A, wherein R² is R²-9.305(=formula 1.315).

Table 316. Compounds of formula I.A, wherein R² is R²-9.306(=formula 1.316).

Table 317. Compounds of formula I.A, wherein R² is R²-9.307(=formula 1.317).

Table 318. Compounds of formula I.A, wherein R² is R²-9.308(=formula 1.318).

Table 319. Compounds of formula I.A, wherein R² is R²-9.309(=formula 1.319).

Table 320. Compounds of formula I.A, wherein R² is R²-9.310(=formula 1.320).

Table 321. Compounds of formula I.A, wherein R² is R²-9.311(=formula 1.321).

Table 322. Compounds of formula I.A, wherein R² is R²-9.312(=formula 1.322).

Table 323. Compounds of formula I.A, wherein R² is R²-9.313(=formula 1.323).

Table 324. Compounds of formula I.A, wherein R² is R²-9.314(=formula 1.324).

Table 325. Compounds of formula I.A, wherein R² is R²-9.315(=formula 1.325).

Table 326. Compounds of formula I.A, wherein R² is R²-9.316(=formula 1.326).

Table 327. Compounds of formula I.A, wherein R² is R²-9.317(=formula 1.327).

Table 328. Compounds of formula I.A, wherein R² is R²-9.318(=formula 1.328).

Table 329. Compounds of formula I.A, wherein R² is R²-9.319(=formula 1.329).

Table 330. Compounds of formula I.A, wherein R² is R²-9.320(=formula 1.330).

Table 331. Compounds of formula I.A, wherein R² is R²-9.321(=formula 1.331).

Table 332. Compounds of formula I.A, wherein R² is R²-9.322(=formula 1.332).

Table 333. Compounds of formula I.A, wherein R² is R²-9.323(=formula 1.333).

Table 334. Compounds of formula I.A, wherein R² is R²-9.324(=formula 1.334).

Table 335. Compounds of formula I.A, wherein R² is R²-9.325(=formula 1.335).

Table 336. Compounds of formula I.A, wherein R² is R²-9.326(=formula 1.336).

Table 337. Compounds of formula I.A, wherein R² is R²-9.327(=formula 1.337).

Table 338. Compounds of formula I.A, wherein R² is R²-9.328(=formula 1.338).

Table 339. Compounds of formula I.A, wherein R² is R²-9.329(=formula 1.339).

Table 340. Compounds of formula I.A, wherein R² is R²-9.330(=formula 1.340).

Table 341. Compounds of formula I.A, wherein R² is R²-9.331(=formula 1.341).

Table 342. Compounds of formula I.A, wherein R² is R²-9.332(=formula 1.342).

Table 343. Compounds of formula I.A, wherein R² is R²-9.333(=formula 1.343).

Table 344. Compounds of formula I.A, wherein R² is R²-9.334(=formula 1.344).

Table 345. Compounds of formula I.A, wherein R² is R²-9.335(=formula 1.345).

Table 346. Compounds of formula I.A, wherein R² is R²-9.336(=formula 1.346).

Table 347. Compounds of formula I.A, wherein R² is R²-9.334(=formula 1.347).

Table 348. Compounds of formula I.A, wherein R² is R²-9.338(=formula 1.348).

Table 349. Compounds of formula I.A, wherein R² is R²-9.339(=formula 1.349).

Table 350. Compounds of formula I.A, wherein R² is R²-9.340(=formula 1.350).

Table 351. Compounds of formula I.A, wherein R² is R²-9.341(=formula 1.351).

Table 352. Compounds of formula I.A, wherein R² is R²-9.342(=formula 1.352).

Table 353. Compounds of formula I.A, wherein R² is R²-9.343(=formula 1.353).

Table 354. Compounds of formula I.A, wherein R² is R²-9.344(=formula 1.354).

Table 355. Compounds of formula I.A, wherein R² is R²-9.345(=formula 1.355).

Table 356. Compounds of formula I.A, wherein R² is R²-9.346(=formula 1.356).

Table 357. Compounds of formula I.A, wherein R² is R²-9.347(=formula 1.357).

Table 358. Compounds of formula I.A, wherein R² is R²-9.348(=formula 1.358).

Table 359. Compounds of formula I.A, wherein R² is R²-9.349(=formula 1.359).

Table 360. Compounds of formula I.A, wherein R² is R²-9.350(=formula 1.360).

Table 361. Compounds of formula I.A, wherein R² is R²-9.351(=formula 1.361).

Table 362. Compounds of formula I.A, wherein R² is R²-9.352(=formula 1.362).

Table 363. Compounds of formula I.A, wherein R² is R²-9.353(=formula 1.363).

Table 364. Compounds of formula I.A, wherein R² is R²-9.354(=formula 1.364).

Table 365. Compounds of formula I.A, wherein R² is R²-9.355(=formula 1.365).

Table 366. Compounds of formula I.A, wherein R² is R²-9.356(=formula 1.366).

Table 367. Compounds of formula I.A, wherein R² is R²-9.357(=formula 1.367).

Table 368. Compounds of formula I.A, wherein R² is R²-9.358(=formula 1.368).

Table 369. Compounds of formula I.A, wherein R² is R²-9.359(=formula 1.369).

Table 370. Compounds of formula I.A, wherein R² is R²-9.360(=formula 1.370).

Table 371. Compounds of formula I.A, wherein R² is R²-9.361(=formula 1.371).

Table 372. Compounds of formula I.A, wherein R² is R²-9.362(=formula 1.372).

Table 373. Compounds of formula I.A, wherein R² is R²-9.363(=formula 1.373).

Table 374. Compounds of formula I.A, wherein R² is R²-9.364(=formula 1.374).

Table 375. Compounds of formula I.A, wherein R² is R²-9.365(=formula 1.375).

Table 376. Compounds of formula I.A, wherein R² is R²-9.366(=formula 1.376).

Table 377. Compounds of formula I.A, wherein R² is R²-9.367(=formula 1.377).

Table 378. Compounds of formula I.A, wherein R² is R²-9.368(=formula 1.378).

Table 379. Compounds of formula I.A, wherein R² is R²-9.369(=formula 1.379).

Table 380. Compounds of formula I.A, wherein R² is R²-9.370(=formula 1.380).

Table 381. Compounds of formula I.A, wherein R² is R²-9.371(=formula 1.381).

Table 382. Compounds of formula I.A, wherein R² is R²-9.372(=formula 1.382).

Table 383. Compounds of formula I.A, wherein R² is R²-9.373(=formula 1.383).

Table 384. Compounds of formula I.A, wherein R² is R²-9.374(=formula 1.384).

Table 385. Compounds of formula I.A, wherein R² is R²-9.375(=formula 1.385).

Table 386. Compounds of formula I.A, wherein R² is R²-9.376(=formula 1.386).

Table 387. Compounds of formula I.A, wherein R² is R²-9.377(=formula 1.387).

Table 388. Compounds of formula I.A, wherein R² is R²-9.378(=formula 1.388).

Table 389. Compounds of formula I.A, wherein R² is R²-9.379(=formula 1.389).

Table 390. Compounds of formula I.A, wherein R² is R²-9.380(=formula 1.390).

Table 391. Compounds of formula I.A, wherein R² is R²-9.381(=formula 1.391).

Table 392. Compounds of formula I.A, wherein R² is R²-9.382(=formula 1.392).

Table 393. Compounds of formula I.A, wherein R² is R²-9.383(=formula 1.393).

Table 394. Compounds of formula I.A, wherein R² is R²-9.384(=formula 1.394).

Table 395. Compounds of formula I.A, wherein R² is R²-9.385(=formula 1.395).

Table 396. Compounds of formula I.A, wherein R² is R²-9.386(=formula 1.396).

Table 397. Compounds of formula I.A, wherein R² is R²-9.387(=formula 1.397).

Table 398. Compounds of formula I.A, wherein R² is R²-9.388(=formula 1.398).

Table 399. Compounds of formula I.A, wherein R² is R²-9.389(=formula 1.399).

Table 400. Compounds of formula I.A, wherein R² is R²-9.390(=formula 1.400).

Table 401. Compounds of formula I.A, wherein R² is R²-9.391(=formula 1.401).

Table 402. Compounds of formula I.A, wherein R² is R²-9.392(=formula 1.402).

Table 403. Compounds of formula I.A, wherein R² is R²-9.393(=formula 1.403).

Table 404. Compounds of formula I.A, wherein R² is R²-9.394(=formula 1.404).

Table 405. Compounds of formula I.A, wherein R² is R²-9.395(=formula 1.405).

Table 406. Compounds of formula I.A, wherein R² is R²-9.396(=formula 1.406).

Table 407. Compounds of formula I.A, wherein R² is R²-9.397(=formula 1.407).

Table 408. Compounds of formula I.A, wherein R² is R²-9.398(=formula 1.408).

Table 409. Compounds of formula I.A, wherein R² is R²-9.399(=formula 1.409).

Table 410. Compounds of formula I.A, wherein R² is R²-9.400(=formula 1.410).

Table 411. Compounds of formula I.A, wherein R² is R²-9.401(=formula 1.411).

Table 412. Compounds of formula I.A, wherein R² is R²-9.402(=formula 1.412).

Table 413. Compounds of formula I.A, wherein R² is R²-9.403(=formula 1.413).

Table 414. Compounds of formula I.A, wherein R² is R²-9.404(=formula 1.414).

Table 415. Compounds of formula I.A, wherein R² is R²-9.405(=formula 1.415).

Table 416. Compounds of formula I.A, wherein R² is R²-9.406(=formula 1.416).

Table 417. Compounds of formula I.A, wherein R² is R²-9.407(=formula 1.417).

Table 418. Compounds of formula I.A, wherein R² is R²-9.408(=formula 1.418).

Table 419. Compounds of formula I.A, wherein R² is R²-9.409(=formula 1.419).

Table 420. Compounds of formula I.A, wherein R² is R²-9.410(=formula 1.420).

Table 421. Compounds of formula I.A, wherein R² is R²-9.411(=formula 1.421).

Table 422. Compounds of formula I.A, wherein R² is R²-9.412(=formula 1.422).

Table 423. Compounds of formula I.A, wherein R² is R²-9.413(=formula 1.423).

Table 424. Compounds of formula I.A, wherein R² is R²-9.414(=formula 1.424).

Table 425. Compounds of formula I.A, wherein R² is R²-9.415(=formula 1.425).

Table 426. Compounds of formula I.A, wherein R² is R²-9.416(=formula 1.426).

Table 427. Compounds of formula I.A, wherein R² is R²-9.417(=formula 1.427).

Table 428. Compounds of formula I.A, wherein R² is R²-9.418(=formula 1.428).

Table 429. Compounds of formula I.A, wherein R² is R²-9.419(=formula 1.429).

Table 430. Compounds of formula I.A, wherein R² is R²-9.420(=formula 1.430).

Table 431. Compounds of formula I.A, wherein R² is R²-9.421(=formula 1.431).

Table 432. Compounds of formula I.A, wherein R² is R²-9.422(=formula 1.432).

Table 433. Compounds of formula I.A, wherein R² is R²-9.423(=formula 1.433).

Table 434. Compounds of formula I.A, wherein R² is R²-9.424(=formula 1.434).

Table 435. Compounds of formula I.A, wherein R² is R²-9.425(=formula 1.435).

Table 436. Compounds of formula I.A, wherein R² is R²-9.426(=formula 1.436).

Table 437. Compounds of formula I.A, wherein R² is R²-9.427(=formula 1.437).

Table 438. Compounds of formula I.A, wherein R² is R²-9.428(=formula 1.438).

Table 439. Compounds of formula I.A, wherein R² is R²-9.429(=formula 1.439).

Table 440. Compounds of formula I.A, wherein R² is R²-9.430(=formula 1.440).

Table 441. Compounds of formula I.A, wherein R² is R²-9.431(=formula 1.441).

Table 442. Compounds of formula I.A, wherein R² is R²-9.432(=formula 1.442).

Table 443. Compounds of formula I.A, wherein R² is R²-9.433(=formula 1.443).

Table 444. Compounds of formula I.A, wherein R² is R²-9.434(=formula 1.444).

Table 445. Compounds of formula I.A, wherein R² is R²-9.435(=formula 1.445).

Table 446. Compounds of formula I.A, wherein R² is R²-9.436(=formula 1.446).

Table 447. Compounds of formula I.A, wherein R² is R²-9.437(=formula 1.447).

Table 448. Compounds of formula I.A, wherein R² is R²-9.438(=formula 1.448).

Table 449. Compounds of formula I.A, wherein R² is R²-9.439(=formula 1.449).

Table 450. Compounds of formula I.A, wherein R² is R²-9.440(=formula 1.450).

Table 451. Compounds of formula I.A, wherein R² is R²-9.441(=formula 1.451).

Table 452. Compounds of formula I.A, wherein R² is R²-9.442(=formula 1.452).

Table 453. Compounds of formula I.A, wherein R² is R²-9.443(=formula 1.453).

Table 454. Compounds of formula I.A, wherein R² is R²-9.444(=formula 1.454).

Table 455. Compounds of formula I.A, wherein R² is R²-9.445(=formula 1.455).

Table 456. Compounds of formula I.A, wherein R² is R²-9.446(=formula 1.456).

Table 457. Compounds of formula I.A, wherein R² is R²-9.447(=formula 1.457).

Table 458. Compounds of formula I.A, wherein R² is R²-9.448(=formula 1.458).

Table 459. Compounds of formula I.A, wherein R² is R²-9.449(=formula 1.459).

Table 460. Compounds of formula I.A, wherein R² is R²-9.450(=formula 1.460).

Table 461. Compounds of formula I.A, wherein R² is R²-9.451(=formula 1.461).

Table 462. Compounds of formula I.A, wherein R² is R²-9.452(=formula 1.462).

Table 463. Compounds of formula I.A, wherein R² is R²-9.453(=formula 1.463).

Table 464. Compounds of formula I.A, wherein R² is R²-9.454(=formula 1.464).

Table 465. Compounds of formula I.A, wherein R² is R²-9.455(=formula 1.465).

Table 466. Compounds of formula I.A, wherein R² is R²-9.456(=formula 1.466).

Table 467. Compounds of formula I.A, wherein R² is R²-9.457(=formula 1.467).

Table 468. Compounds of formula I.A, wherein R² is R²-9.458(=formula 1.468).

Table 469. Compounds of formula I.A, wherein R² is R²-9.459(=formula 1.469).

Table 470. Compounds of formula I.A, wherein R² is R²-9.460(=formula 1.470).

Table 471. Compounds of formula I.A, wherein R² is R²-9.461(=formula 1.471).

Table 472. Compounds of formula I.A, wherein R² is R²-9.462(=formula 1.472).

Table 473. Compounds of formula I.A, wherein R² is R²-9.463(=formula 1.473).

Table 474. Compounds of formula I.A, wherein R² is R²-9.464(=formula 1.474).

Table 475. Compounds of formula I.A, wherein R² is R²-9.465(=formula 1.475).

Table 476. Compounds of formula I.A, wherein R² is R²-9.466(=formula 1.476).

Table 477. Compounds of formula I.A, wherein R² is R²-9.467(=formula 1.477).

Table 478. Compounds of formula I.A, wherein R² is R²-9.468(=formula 1.478).

Table 479. Compounds of formula I.A, wherein R² is R²-9.469(=formula 1.479).

Table 480. Compounds of formula I.A, wherein R² is R²-9.470(=formula 1.480).

Table 481. Compounds of formula I.A, wherein R² is R²-9.471(=formula 1.481).

Table 482. Compounds of formula I.A, wherein R² is R²-9.472(=formula 1.482).

Table 483. Compounds of formula I.A, wherein R² is R²-9.473(=formula 1.483).

Table 484. Compounds of formula I.A, wherein R² is R²-9.474(=formula 1.484).

Table 485. Compounds of formula I.A, wherein R² is R²-9.475(=formula 1.485).

Table 486. Compounds of formula I.A, wherein R² is R²-9.476(=formula 1.486).

Table 487. Compounds of formula I.A, wherein R² is R²-9.477(=formula 1.487).

Table 488. Compounds of formula I.A, wherein R² is R²-9.478(=formula 1.488).

Table 489. Compounds of formula I.A, wherein R² is R²-9.479(=formula 1.489).

Table 490. Compounds of formula I.A, wherein R² is R²-9.480(=formula 1.490).

Table 491. Compounds of formula I.A, wherein R² is R²-9.481(=formula 1.491).

Table 492. Compounds of formula I.A, wherein R² is R²-9.482(=formula 1.492).

Table 493. Compounds of formula I.A, wherein R² is R²-9.483(=formula 1.493).

Table 494. Compounds of formula I.A, wherein R² is R²-9.484(=formula 1.494).

Table 495. Compounds of formula I.A, wherein R² is R²-9.485(=formula 1.495).

Table 496. Compounds of formula I.A, wherein R² is R²-9.486(=formula 1.496).

Table 497. Compounds of formula I.A, wherein R² is R²-9.487(=formula 1.497).

Table 498. Compounds of formula I.A, wherein R² is R²-9.488(=formula 1.498).

Table 499. Compounds of formula I.A, wherein R² is R²-9.489(=formula 1.499).

Table 500. Compounds of formula I.A, wherein R² is R²-9.490(=formula 1.500).

Table 501. Compounds of formula I.A, wherein R² is R²-9.491(=formula 1.501).

Table 502. Compounds of formula I.A, wherein R² is R²-9.492 (=formula 1.502).

Table 503. Compounds of formula I.A, wherein R² is R²-9.493(=formula 1.503).

Table 504. Compounds of formula I.A, wherein R² is R²-9.494(=formula 1.504).

Table 505. Compounds of formula I.A, wherein R² is R²-9.495(=formula 1.505).

Table 506. Compounds of formula I.A, wherein R² is R²-9.496(=formula 1.506).

Table 507. Compounds of formula I.A, wherein R² is R²-9.497(=formula 1.507).

Table 508. Compounds of formula I.A, wherein R² is R²-9.498(=formula 1.508).

Table 509. Compounds of formula I.A, wherein R² is R²-9.499(=formula 1.509).

Table 510. Compounds of formula I.A, wherein R² is R²-9.500(=formula 1.510).

Table 511. Compounds of formula I.A, wherein R² is R²-9.501(=formula 1.511).

Table 512. Compounds of formula I.A, wherein R² is R²-9.502(=formula 1.512).

Table 513. Compounds of formula I.A, wherein R² is R²-9.503(=formula 1.513).

Table 514. Compounds of formula I.A, wherein R² is R²-9.504(=formula 1.514).

Table 515. Compounds of formula I.A, wherein R² is R²-9.505(=formula 1.515).

Table 516. Compounds of formula I.A, wherein R² is R²-9.506(=formula 1.516).

Table 517. Compounds of formula I.A, wherein R² is R²-9.507(=formula 1.517).

Table 518. Compounds of formula I.A, wherein R² is R²-9.508(=formula 1.518).

Table 519. Compounds of formula I.A, wherein R² is R²-9.509(=formula 1.519).

Table 520. Compounds of formula I.A, wherein R² is R²-9.510(=formula 1.520).

Table 521. Compounds of formula I.A, wherein R² is R²-9.511(=formula 1.521).

Table 522. Compounds of formula I.A, wherein R² is R²-9.512 (=formula 1.522).

Table 523. Compounds of formula I.A, wherein R² is R²-9.513 (=formula 1.523).

Table 524. Compounds of formula I.A, wherein R² is R²-9.514(=formula 1.524).

Table 525. Compounds of formula I.A, wherein R² is R²-9.515(=formula 1.525).

Table 526. Compounds of formula I.A, wherein R² is R²-9.516(=formula 1.526).

Table 527. Compounds of formula I.A, wherein R² is R²-9.517(=formula 1.527).

Table 528. Compounds of formula I.A, wherein R² is R²-9.518(=formula 1.528).

Table 529. Compounds of formula I.A, wherein R² is R²-9.519(=formula 1.529).

Table 530. Compounds of formula I.A, wherein R² is R²-9.520(=formula 1.530).

Table 531. Compounds of formula I.A, wherein R² is R²-9.521(=formula 1.531).

Table 532. Compounds of formula I.A, wherein R² is R²-9.522(=formula 1.532).

Table 533. Compounds of formula I.A, wherein R² is R²-9.523(=formula 1.533).

Table 534. Compounds of formula I.A, wherein R² is R²-9.524(=formula 1.534).

Table 535. Compounds of formula I.A, wherein R² is R²-9.525(=formula 1.535).

Table 536. Compounds of formula I.A, wherein R² is R²-9.526(=formula 1.536).

Table 537. Compounds of formula I.A, wherein R² is R²-9.527(=formula 1.537).

Table 538. Compounds of formula I.A, wherein R² is R²-9.528(=formula 1.538).

Table 539. Compounds of formula I.A, wherein R² is R²-9.529(=formula 1.539).

Table 540. Compounds of formula I.A, wherein R² is R²-9.530(=formula 1.540).

Table 541. Compounds of formula I.A, wherein R² is R²-9.531(=formula 1.541).

Table 542. Compounds of formula I.A, wherein R² is R²-9.532(=formula 1.542).

Table 543. Compounds of formula I.A, wherein R² is R²-9.533(=formula 1.543).

Table 544. Compounds of formula I.A, wherein R² is R²-9.534(=formula 1.544).

Table 545. Compounds of formula I.A, wherein R² is R²-9.535(=formula 1.545).

Table 546. Compounds of formula I.A, wherein R² is R²-9.536(=formula 1.546).

Table 547. Compounds of formula I.A, wherein R² is R²-9.537(=formula 1.547).

Table 548. Compounds of formula I.A, wherein R² is R²-9.538(=formula 1.548).

Table 549. Compounds of formula I.A, wherein R² is R²-9.539(=formula 1.549).

Table 550. Compounds of formula I.A, wherein R² is R²-9.540(=formula 1.550).

Table 551. Compounds of formula I.A, wherein R² is R²-9.541(=formula 1.551).

Table 552. Compounds of formula I.A, wherein R² is R²-9.542(=formula 1.552).

Table 553. Compounds of formula I.A, wherein R² is R²-9.543(=formula 1.553).

Table 554. Compounds of formula I.A, wherein R² is R²-9.544(=formula 1.554).

Table 555. Compounds of formula I.A, wherein R² is R²-9.545(=formula 1.555).

Table 556. Compounds of formula I.A, wherein R² is R²-9.546(=formula 1.556).

Table 557. Compounds of formula I.A, wherein R² is R²-9.547(=formula 1.557).

Table 558. Compounds of formula I.A, wherein R² is R²-9.548(=formula 1.558).

Table 559. Compounds of formula I.A, wherein R² is R²-9.549(=formula 1.559).

Table 560. Compounds of formula I.A, wherein R² is R²-9.550(=formula 1.560).

Table 561. Compounds of formula I.A, wherein R² is R²-9.551(=formula 1.561).

Table 562. Compounds of formula I.A, wherein R² is R²-9.552(=formula 1.562).

Table 563. Compounds of formula I.A, wherein R² is R²-9.553(=formula 1.563).

Table 564. Compounds of formula I.A, wherein R² is R²-9.554(=formula 1.564).

Table 565. Compounds of formula I.A, wherein R² is R²-9.555(=formula 1.565).

Table 566. Compounds of formula I.A, wherein R² is R²-9.556(=formula 1.566).

Table 567. Compounds of formula I.A, wherein R² is R²-9.557(=formula 1.567).

Table 568. Compounds of formula I.A, wherein R² is R²-9.558(=formula 1.568).

Table 569. Compounds of formula I.A, wherein R² is R²-9.559(=formula 1.569).

Table 570. Compounds of formula I.A, wherein R² is R²-9.560 (=formula 1.570).

Table 571. Compounds of formula I.A, wherein R² is R²-9.561(=formula 1.571).

Table 572. Compounds of formula I.A, wherein R² is R²-9.562(=formula 1.572).

Table 573. Compounds of formula I.A, wherein R² is R²-9.563(=formula 1.573).

Table 574. Compounds of formula I.A, wherein R² is R²-9.564(=formula 1.574).

Table 575. Compounds of formula I.A, wherein R² is R²-9.565(=formula 1.575).

Table 576. Compounds of formula I.A, wherein R² is R²-9.566(=formula 1.576).

Table 577. Compounds of formula I.A, wherein R² is R²-9.567(=formula 1.577).

Table 578. Compounds of formula I.A, wherein R² is R²-9.568(=formula 1.578).

Table 579. Compounds of formula I.A, wherein R² is R²-9.569(=formula 1.579).

Table 580. Compounds of formula I.A, wherein R² is R²-9.570(=formula 1.580).

Table 581. Compounds of formula I.A, wherein R² is R²-9.571(=formula 1.581).

Table 582. Compounds of formula I.A, wherein R² is R²-9.572(=formula 1.582).

Table 583. Compounds of formula I.A, wherein R² is R²-9.573(=formula 1.583).

Table 584. Compounds of formula I.A, wherein R² is R²-9.574(=formula 1.584).

Table 585. Compounds of formula I.A, wherein R² is R²-9.575(=formula 1.585).

Table 586. Compounds of formula I.A, wherein R² is R²-9.576(=formula 1.586).

Table 587. Compounds of formula I.A, wherein R² is R²-9.577(=formula 1.587).

Table 588. Compounds of formula I.A, wherein R² is R²-9.578(=formula 1.588).

Table 589. Compounds of formula I.A, wherein R² is R²-9.579(=formula 1.589).

Table 590. Compounds of formula I.A, wherein R² is R²-9.580(=formula 1.590).

Table 591. Compounds of formula I.A, wherein R² is R²-9.581(=formula 1.591).

Table 592. Compounds of formula I.A, wherein R² is R²-9.582(=formula 1.592).

Table 593. Compounds of formula I.A, wherein R² is R²-9.583(=formula 1.593).

Table 594. Compounds of formula I.A, wherein R² is R²-9.584(=formula 1.594).

Table 595. Compounds of formula I.A, wherein R² is R²-9.585(=formula 1.595).

Table 596. Compounds of formula I.A, wherein R² is R²-9.586(=formula 1.596).

Table 597. Compounds of formula I.A, wherein R² is R²-9.587(=formula 1.597).

Table 598. Compounds of formula I.A, wherein R² is R²-9.588(=formula 1.598).

Table 599. Compounds of formula I.A, wherein R² is R²-9.589(=formula 1.599).

Table 600. Compounds of formula I.A, wherein R² is R²-9.590(=formula 1.600).

Table 601. Compounds of formula I.A, wherein R² is R²-9.591(=formula 1.601).

Table 602. Compounds of formula I.A, wherein R² is R²-9.592(=formula 1.602).

Table 603. Compounds of formula I.A, wherein R² is R²-9.593(=formula 1.603).

Table 604. Compounds of formula I.A, wherein R² is R²-9.594(=formula 1.604).

Table 605. Compounds of formula I.A, wherein R² is R²-9.595(=formula 1.605).

Table 606. Compounds of formula I.A, wherein R² is R²-9.596(=formula 1.606).

Table 607. Compounds of formula I.A, wherein R² is R²-9.597(=formula 1.607).

Table 608. Compounds of formula I.A, wherein R² is R²-9.598(=formula 1.608).

Table 609. Compounds of formula I.A, wherein R² is R²-9.599(=formula 1.609).

Table 610. Compounds of formula I.A, wherein R² is R²-9.600(=formula 1.610).

Table 611. Compounds of formula I.A, wherein R² is R²-9.601(=formula 1.611).

Table 612. Compounds of formula I.A, wherein R² is R²-9.602(=formula 1.612).

Table 613. Compounds of formula I.A, wherein R² is R²-9.603(=formula 1.613).

Table 614. Compounds of formula I.A, wherein R² is R²-9.604(=formula 1.614).

Table 615. Compounds of formula I.A, wherein R² is R²-9.605(=formula 1.615).

Table 616. Compounds of formula I.A, wherein R² is R²-9.606(=formula 1.616).

Table 617. Compounds of formula I.A, wherein R² is R²-9.607(=formula 1.617).

Table 618. Compounds of formula I.A, wherein R² is R²-9.608(=formula 1.618).

Table 619. Compounds of formula I.A, wherein R² is R²-9.609(=formula 1.619).

Table 620. Compounds of formula I.A, wherein R² is R²-9.610(=formula 1.620).

Table 621. Compounds of formula I.A, wherein R² is R²-9.611(=formula 1.621).

Table 622. Compounds of formula I.A, wherein R² is R²-9.612(=formula 1.622).

Table 623. Compounds of formula I.A, wherein R² is R²-9.613(=formula 1.623).

Table 624. Compounds of formula I.A, wherein R² is R²-9.614(=formula 1.624).

Table 625. Compounds of formula I.A, wherein R² is R²-9.615(=formula 1.625).

Table 626. Compounds of formula I.A, wherein R² is R²-9.616(=formula 1.626).

Table 627. Compounds of formula I.A, wherein R² is R²-9.617(=formula 1.627).

Table 628. Compounds of formula I.A, wherein R² is R²-9.618(=formula 1.628).

Table 629. Compounds of formula I.A, wherein R² is R²-9.619(=formula 1.629).

Table 630. Compounds of formula I.A, wherein R² is R²-9.620(=formula 1.630).

Table 631. Compounds of formula I.A, wherein R² is R²-9.621(=formula 1.631).

Table 632. Compounds of formula I.A, wherein R² is R²-9.622(=formula 1.632).

Table 633. Compounds of formula I.A, wherein R² is R²-9.623(=formula 1.633).

Table 634. Compounds of formula I.A, wherein R² is R²-9.624(=formula 1.634).

Table 635. Compounds of formula I.A, wherein R² is R²-9.625(=formula 1.635).

Table 636. Compounds of formula I.A, wherein R² is R²-9.626(=formula 1.636).

Table 637. Compounds of formula I.A, wherein R² is R²-9.627(=formula 1.637).

Table 638. Compounds of formula I.A, wherein R² is R²-9.628(=formula 1.638).

Table 639. Compounds of formula I.A, wherein R² is R²-9.629(=formula 1.639).

Table 640. Compounds of formula I.A, wherein R² is R²-9.630(=formula 1.640).

Table 641. Compounds of formula I.A, wherein R² is R²-9.631(=formula 1.641).

Table 642. Compounds of formula I.A, wherein R² is R²-9.632(=formula 1.642).

Table 643. Compounds of formula I.A, wherein R² is R²-9.633(=formula 1.643).

Table 644. Compounds of formula I.A, wherein R² is R²-9.634(=formula 1.644).

Table 645. Compounds of formula I.A, wherein R² is R²-9.635(=formula 1.645).

Table 646. Compounds of formula I.A, wherein R² is R²-9.636(=formula 1.646).

Table 647. Compounds of formula I.A, wherein R² is R²-9.637(=formula 1.647).

Table 648. Compounds of formula I.A, wherein R² is R²-9.638(=formula 1.648).

Table 649. Compounds of formula I.A, wherein R² is R²-9.639(=formula 1.649).

Table 650. Compounds of formula I.A, wherein R² is R²-9.640(=formula 1.650).

Table 651. Compounds of formula I.A, wherein R² is R²-9.641(=formula 1.651).

Table 652. Compounds of formula I.A, wherein R² is R²-9.642(=formula 1.652).

Table 653. Compounds of formula I.A, wherein R² is R²-9.643(=formula 1.653).

Table 654. Compounds of formula I.A, wherein R² is R²-9.644(=formula 1.654).

Table 655. Compounds of formula I.A, wherein R² is R²-9.645(=formula 1.655).

Table 656. Compounds of formula I.A, wherein R² is R²-9.646(=formula 1.656).

Table 657. Compounds of formula I.A, wherein R² is R²-9.647(=formula 1.657).

Table 658. Compounds of formula I.A, wherein R² is R²-9.648(=formula 1.658).

Table 659. Compounds of formula I.A, wherein R² is R²-9.649(=formula 1.659).

Table 660. Compounds of formula I.A, wherein R² is R²-9.650(=formula 1.660).

Table 661. Compounds of formula I.A, wherein R² is R²-9.651(=formula 1.661).

Table 662. Compounds of formula I.A, wherein R² is R²-9.652(=formula 1.662).

Table 663. Compounds of formula I.A, wherein R² is R²-9.653(=formula 1.663).

Table 664. Compounds of formula I.A, wherein R² is R²-9.654(=formula 1.664).

Table 665. Compounds of formula I.A, wherein R² is R²-9.655(=formula 1.665).

Table 666. Compounds of formula I.A, wherein R² is R²-9.656(=formula 1.666).

Table 667. Compounds of formula I.A, wherein R² is R²-9.657(=formula 1.667).

Table 668. Compounds of formula I.A, wherein R² is R²-9.658(=formula 1.668).

Table 669. Compounds of formula I.A, wherein R² is R²-9.659(=formula 1.669).

Table 670. Compounds of formula I.A, wherein R² is R²-9.660(=formula 1.670).

Table 671. Compounds of formula I.A, wherein R² is R²-9.661(=formula 1.671).

Table 672. Compounds of formula I.A, wherein R² is R²-9.662(=formula 1.672).

Table 673. Compounds of formula I.A, wherein R² is R²-9.663(=formula 1.673).

Table 674. Compounds of formula I.A, wherein R² is R²-9.664(=formula 1.674).

Table 675. Compounds of formula I.A, wherein R² is R²-9.665(=formula 1.675).

Table 676. Compounds of formula I.A, wherein R² is R²-9.666(=formula 1.676).

Table 677. Compounds of formula I.A, wherein R² is R²-9.667(=formula 1.677).

Table 678. Compounds of formula I.A, wherein R² is R²-9.668(=formula 1.678).

Table 679. Compounds of formula I.A, wherein R² is R²-9.669(=formula 1.679).

Table 680. Compounds of formula I.A, wherein R² is R²-9.670(=formula 1.680).

Table 681. Compounds of formula I.A, wherein R² is R²-9.671(=formula 1.681).

Table 682. Compounds of formula I.A, wherein R² is R²-9.672(=formula 1.682).

Table 683. Compounds of formula I.A, wherein R² is R²-9.673(=formula 1.683).

Table 684. Compounds of formula I.A, wherein R² is R²-9.674(=formula 1.684).

Table 685. Compounds of formula I.A, wherein R² is R²-9.675(=formula 1.685).

Table 686. Compounds of formula I.A, wherein R² is R²-9.676(=formula 1.686).

Table 687. Compounds of formula I.A, wherein R² is R²-10.1(=formula 1.687).

Table 688. Compounds of formula I.A, wherein R² is R²-10.2(=formula 1.688).

Table 689. Compounds of formula I.A, wherein R² is R²-10.3(=formula 1.689).

Table 690. Compounds of formula I.A, wherein R² is R²-10.4(=formula 1.690).

Table 691. Compounds of formula I.A, wherein R² is R²-10.5(=formula 1.691).

Table 692. Compounds of formula I.A, wherein R² is R²-10.6(=formula 1.692).

Table 693. Compounds of formula I.A, wherein R² is R²-10.7(=formula 1.693).

Table 694. Compounds of formula I.A, wherein R² is R²-10.8(=formula 1.694).

Table 695. Compounds of formula I.A, wherein R² is R²-10.9(=formula 1.695).

Table 696. Compounds of formula I.A, wherein R² is R²-10.10(=formula 1.696).

Table 697. Compounds of formula I.A, wherein R² is R²-10.11(=formula 1.697).

Table 698. Compounds of formula I.A, wherein R² is R²-10.12(=formula 1.698).

Table 699. Compounds of formula I.A, wherein R² is R²-10.13(=formula 1.699).

Table 700. Compounds of formula I.A, wherein R² is R²-10.14(=formula 1.700).

Table 701. Compounds of formula I.A, wherein R² is R²-10.15(=formula 1.701).

Table 702. Compounds of formula I.A, wherein R² is R²-10.16(=formula 1.702).

Table 703. Compounds of formula I.A, wherein R² is R²-10.17(=formula 1.703).

Table 704. Compounds of formula I.A, wherein R² is R²-10.18(=formula 1.704).

Table 705. Compounds of formula I.A, wherein R² is R²-10.19(=formula 1.705).

Table 706. Compounds of formula I.A, wherein R² is R²-10.20(=formula 1.706).

Table 707. Compounds of formula I.A, wherein R² is R²-10.21(=formula 1.707).

Table 708. Compounds of formula I.A, wherein R² is R²-10.22(=formula 1.708).

Table 709. Compounds of formula I.A, wherein R² is R²-10.23(=formula 1.709).

Table 710. Compounds of formula I.A, wherein R² is R²-10.24(=formula 1.710).

Table 711. Compounds of formula I.A, wherein R² is R²-10.25(=formula 1.711).

Table 712. Compounds of formula I.A, wherein R² is R²-10.26(=formula 1.712).

Table 713. Compounds of formula I.A, wherein R² is R²-10.27(=formula 1.713).

Table 714. Compounds of formula I.A, wherein R² is R²-10.28(=formula 1.714).

Table 715. Compounds of formula I.A, wherein R² is R²-10.29(=formula 1.715).

Table 716. Compounds of formula I.A, wherein R² is R²-10.30(=formula 1.716).

Table 717. Compounds of formula I.A, wherein R² is R²-10.31(=formula 1.717).

Table 718. Compounds of formula I.A, wherein R² is R²-10.32(=formula 1.718).

Table 719. Compounds of formula I.A, wherein R² is R²-10.33(=formula 1.719).

Table 720. Compounds of formula I.A, wherein R² is R²-10.34(=formula 1.720).

Table 721. Compounds of formula I.A, wherein R² is R²-10.35(=formula 1.721).

Table 722. Compounds of formula I.A, wherein R² is R²-10.36(=formula 1.722).

Table 723. Compounds of formula I.A, wherein R² is R²-10.37(=formula 1.723).

Table 724. Compounds of formula I.A, wherein R² is R²-10.38(=formula 1.724).

Table 725. Compounds of formula I.A, wherein R² is R²-10.39(=formula 1.725).

Table 726. Compounds of formula I.A, wherein R² is R²-10.40(=formula 1.726).

Table 727. Compounds of formula I.A, wherein R² is R²-10.41(=formula 1.727).

Table 728. Compounds of formula I.A, wherein R² is R²-10.42(=formula 1.728).

Table 729. Compounds of formula I.A, wherein R² is R²-10.43(=formula 1.729).

Table 730. Compounds of formula I.A, wherein R² is R²-10.44(=formula 1.730).

Table 731. Compounds of formula I.A, wherein R² is R²-10.45(=formula 1.731).

Table 732. Compounds of formula I.A, wherein R² is R²-10.46(=formula 1.732).

Table 733. Compounds of formula I.A, wherein R² is R²-10.47(=formula 1.733).

Table 734. Compounds of formula I.A, wherein R² is R²-10.48(=formula 1.734).

Table 735. Compounds of formula I.A, wherein R² is R²-10.49(=formula 1.735).

Table 736. Compounds of formula I.A, wherein R² is R²-10.50(=formula 1.736).

Table 737. Compounds of formula I.A, wherein R² is R²-10.51(=formula 1.737).

Table 738. Compounds of formula I.A, wherein R² is R²-10.52(=formula 1.738).

Table 739. Compounds of formula I.A, wherein R² is R²-10.53(=formula 1.739).

Table 740. Compounds of formula I.A, wherein R² is R²-10.54(=formula 1.740).

Table 741. Compounds of formula I.A, wherein R² is R²-10.55(=formula 1.741).

Table 742. Compounds of formula I.A, wherein R² is R²-10.56(=formula 1.742).

Table 743. Compounds of formula I.A, wherein R² is R²-10.57(=formula 1.743).

Table 744. Compounds of formula I.A, wherein R² is R²-10.58(=formula 1.744).

Table 745. Compounds of formula I.A, wherein R² is R²-10.59(=formula 1.745).

Table 746. Compounds of formula I.A, wherein R² is R²-10.60(=formula 1.746).

Table 747. Compounds of formula I.A, wherein R² is R²-10.61(=formula 1.747).

Table 748. Compounds of formula I.A, wherein R² is R²-10.62(=formula 1.748).

Table 749. Compounds of formula I.A, wherein R² is R²-10.63(=formula 1.749).

Table 750. Compounds of formula I.A, wherein R² is R²-10.64(=formula 1.750).

Table 751. Compounds of formula I.A, wherein R² is R²-10.65(=formula 1.751).

Table 752. Compounds of formula I.A, wherein R² is R²-10.66(=formula 1.752).

Table 753. Compounds of formula I.A, wherein R² is R²-10.67(=formula 1.753).

Table 754. Compounds of formula I.A, wherein R² is R²-10.68(=formula 1.754).

Table 755. Compounds of formula I.A, wherein R² is R²-10.69(=formula 1.755).

Table 756. Compounds of formula I.A, wherein R² is R²-10.70(=formula 1.756).

Table 757. Compounds of formula I.A, wherein R² is R²-10.71(=formula 1.757).

Table 758. Compounds of formula I.A, wherein R² is R²-10.72(=formula 1.758).

Table 759. Compounds of formula I.A, wherein R² is R²-10.73(=formula 1.759).

Table 760. Compounds of formula I.A, wherein R² is R²-10.74(=formula 1.760).

Table 761. Compounds of formula I.A, wherein R² is R²-10.75(=formula 1.761).

Table 762. Compounds of formula I.A, wherein R² is R²-10.76(=formula 1.762).

Table 763. Compounds of formula I.A, wherein R² is R²-10.77(=formula 1.763).

Table 764. Compounds of formula I.A, wherein R² is R²-10.78(=formula 1.764).

Table 765. Compounds of formula I.A, wherein R² is R²-10.79(=formula 1.765).

Table 766. Compounds of formula I.A, wherein R² is R²-10.80(=formula 1.766).

Table 767. Compounds of formula I.A, wherein R² is R²-10.81(=formula 1.767).

Table 768. Compounds of formula I.A, wherein R² is R²-10.82(=formula 1.768).

Table 769. Compounds of formula I.A, wherein R² is R²-10.83(=formula 1.769).

Table 770. Compounds of formula I.A, wherein R² is R²-10.84(=formula 1.770).

Table 771. Compounds of formula I.A, wherein R² is R²-10.85(=formula 1.771).

Table 772. Compounds of formula I.A, wherein R² is R²-10.86(=formula 1.772).

Table 773. Compounds of formula I.A, wherein R² is R²-10.87(=formula 1.773).

Table 774. Compounds of formula I.A, wherein R² is R²-10.88(=formula 1.774).

Table 775. Compounds of formula I.A, wherein R² is R²-10.89(=formula 1.775).

Table 776. Compounds of formula I.A, wherein R² is R²-10.90(=formula 1.776).

Table 777. Compounds of formula I.A, wherein R² is R²-10.91(=formula 1.777).

Table 778. Compounds of formula I.A, wherein R² is R²-10.92(=formula 1.778).

Table 779. Compounds of formula I.A, wherein R² is R²-10.93(=formula 1.779).

Table 780. Compounds of formula I.A, wherein R² is R²-10.94(=formula 1.780).

Table 781. Compounds of formula I.A, wherein R² is R²-10.95(=formula 1.781).

Table 782. Compounds of formula I.A, wherein R² is R²-10.96(=formula 1.782).

Table 783. Compounds of formula I.A, wherein R² is R²-10.97(=formula 1.783).

Table 784. Compounds of formula I.A, wherein R² is R²-10.98(=formula 1.784).

Table 785. Compounds of formula I.A, wherein R² is R²-10.99(=formula 1.785).

Table 786. Compounds of formula I.A, wherein R² is R²-10.100(=formula 1.786).

Table 787. Compounds of formula I.A, wherein R² is R²-10.101(=formula 1.787).

Table 788. Compounds of formula I.A, wherein R² is R²-10.102(=formula 1.788).

Table 789. Compounds of formula I.A, wherein R² is R²-10.103(=formula 1.789).

Table 790. Compounds of formula I.A, wherein R² is R²-10.104(=formula 1.790).

Table 791. Compounds of formula I.A, wherein R² is R²-10.105(=formula 1.791).

Table 792. Compounds of formula I.A, wherein R² is R²-10.106(=formula 1.792).

Table 793. Compounds of formula I.A, wherein R² is R²-10.107(=formula 1.793).

Table 794. Compounds of formula I.A, wherein R² is R²-10.108(=formula 1.794).

Table 795. Compounds of formula I.A, wherein R² is R²-10.109(=formula 1.795).

Table 796. Compounds of formula I.A, wherein R² is R²-10.110(=formula 1.796).

Table 797. Compounds of formula I.A, wherein R² is R²-10.111(=formula 1.797).

Table 798. Compounds of formula I.A, wherein R² is R²-10.112(=formula 1.798).

Table 799. Compounds of formula I.A, wherein R² is R²-10.113(=formula 1.799).

Table 800. Compounds of formula I.A, wherein R² is R²-10.114(=formula 1.800).

Table 801. Compounds of formula I.A, wherein R² is R²-10.115(=formula 1.801).

Table 802. Compounds of formula I.A, wherein R² is R²-10.116(=formula 1.802).

Table 803. Compounds of formula I.A, wherein R² is R²-10.117(=formula 1.803).

Table 804. Compounds of formula I.A, wherein R² is R²-10.118(=formula 1.804).

Table 805. Compounds of formula I.A, wherein R² is R²-10.119(=formula 1.805).

Table 806. Compounds of formula I.A, wherein R² is R²-10.120(=formula 1.806).

Table 807. Compounds of formula I.A, wherein R² is R²-10.121(=formula 1.807).

Table 808. Compounds of formula I.A, wherein R² is R²-10.122(=formula 1.808).

Table 809. Compounds of formula I.A, wherein R² is R²-10.123(=formula 1.809).

Table 810. Compounds of formula I.A, wherein R² is R²-10.124(=formula 1.810).

Table 811. Compounds of formula I.A, wherein R² is R²-10.125(=formula 1.811).

Table 812. Compounds of formula I.A, wherein R² is R²-10.126(=formula 1.812).

Table 813. Compounds of formula I.A, wherein R² is R²-10.127(=formula 1.813).

Table 814. Compounds of formula I.A, wherein R² is R²-10.128(=formula 1.814).

Table 815. Compounds of formula I.A, wherein R² is R²-10.129(=formula 1.815).

Table 816. Compounds of formula I.A, wherein R² is R²-10.130(=formula 1.816).

Table 817. Compounds of formula I.A, wherein R² is R²-10.131(=formula 1.817).

Table 818. Compounds of formula I.A, wherein R² is R²-10.132(=formula 1.818).

Table 819. Compounds of formula I.A, wherein R² is R²-10.133(=formula 1.819).

Table 820. Compounds of formula I.A, wherein R² is R²-10.134(=formula 1.820).

Table 821. Compounds of formula I.A, wherein R² is R²-10.135(=formula 1.821).

Table 822. Compounds of formula I.A, wherein R² is R²-10.136(=formula 1.822).

Table 823. Compounds of formula I.A, wherein R² is R²-10.137(=formula 1.823).

Table 824. Compounds of formula I.A, wherein R² is R²-10.138(=formula 1.824).

Table 825. Compounds of formula I.A, wherein R² is R²-10.139(=formula 1.825).

Table 826. Compounds of formula I.A, wherein R² is R²-10.140(=formula 1.826).

Table 827. Compounds of formula I.A, wherein R² is R²-10.141(=formula 1.827).

Table 828. Compounds of formula I.A, wherein R² is R²-10.142(=formula 1.828).

Table 829. Compounds of formula I.A, wherein R² is R²-10.143(=formula 1.829).

Table 830. Compounds of formula I.A, wherein R² is R²-10.144(=formula 1.830).

Table 831. Compounds of formula I.A, wherein R² is R²-10.145(=formula 1.831).

Table 832. Compounds of formula I.A, wherein R² is R²-10.146(=formula 1.832).

Table 833. Compounds of formula I.A, wherein R² is R²-10.147(=formula 1.833).

Table 834. Compounds of formula I.A, wherein R² is R²-10.148(=formula 1.834).

Table 835. Compounds of formula I.A, wherein R² is R²-10.149(=formula 1.835).

Table 836. Compounds of formula I.A, wherein R² is R²-10.150(=formula 1.836).

Table 837. Compounds of formula I.A, wherein R² is R²-10.151(=formula 1.837).

Table 838. Compounds of formula I.A, wherein R² is R²-10.152(=formula 1.838).

Table 839. Compounds of formula I.A, wherein R² is R²-10.153(=formula 1.839).

Table 840. Compounds of formula I.A, wherein R² is R²-10.154(=formula 1.840).

Table 841. Compounds of formula I.A, wherein R² is R²-10.155(=formula 1.841).

Table 842. Compounds of formula I.A, wherein R² is R²-10.156(=formula 1.842).

Table 843. Compounds of formula I.A, wherein R² is R²-10.157(=formula 1.843).

Table 844. Compounds of formula I.A, wherein R² is R²-10.158(=formula 1.844).

Table 845. Compounds of formula I.A, wherein R² is R²-10.159(=formula 1.845).

Table 846. Compounds of formula I.A, wherein R² is R²-10.160(=formula 1.846).

Table 847. Compounds of formula I.A, wherein R² is R²-10.161(=formula 1.847).

Table 848. Compounds of formula I.A, wherein R² is R²-10.162(=formula 1.848).

Table 849. Compounds of formula I.A, wherein R² is R²-10.163(=formula 1.849).

Table 850. Compounds of formula I.A, wherein R² is R²-10.164(=formula 1.850).

Table 851. Compounds of formula I.A, wherein R² is R²-10.165(=formula 1.851).

Table 852. Compounds of formula I.A, wherein R² is R²-10.166(=formula 1.852).

Table 853. Compounds of formula I.A, wherein R² is R²-10.167(=formula 1.853).

Table 854. Compounds of formula I.A, wherein R² is R²-10.168(=formula 1.854).

Table 855. Compounds of formula I.A, wherein R² is R²-10.169(=formula 1.855).

Table 856. Compounds of formula I.A, wherein R² is R²-10.170(=formula 1.856).

Table 857. Compounds of formula I.A, wherein R² is R²-10.171(=formula 1.857).

Table 858. Compounds of formula I.A, wherein R² is R²-10.172(=formula 1.858).

Table 859. Compounds of formula I.A, wherein R² is R²-10.173(=formula 1.859).

Table 860. Compounds of formula I.A, wherein R² is R²-10.174(=formula 1.860).

Table 861. Compounds of formula I.A, wherein R² is R²-10.175(=formula 1.861).

Table 862. Compounds of formula I.A, wherein R² is R²-10.176(=formula 1.862).

Table 863. Compounds of formula I.A, wherein R² is R²-10.177(=formula 1.863).

Table 864. Compounds of formula I.A, wherein R² is R²-10.178(=formula 1.864).

Table 865. Compounds of formula I.A, wherein R² is R²-10.179(=formula 1.865).

Table 866. Compounds of formula I.A, wherein R² is R²-10.180(=formula 1.866).

Table 867. Compounds of formula I.A, wherein R² is R²-10.181(=formula 1.867).

Table 868. Compounds of formula I.A, wherein R² is R²-10.182(=formula 1.868).

Table 869. Compounds of formula I.A, wherein R² is R²-10.183(=formula 1.869).

Table 870. Compounds of formula I.A, wherein R² is R²-10.184(=formula 1.870).

Table 871. Compounds of formula I.A, wherein R² is R²-10.185(=formula 1.871).

Table 872. Compounds of formula I.A, wherein R² is R²-10.186(=formula 1.872).

Table 873. Compounds of formula I.A, wherein R² is R²-10.187(=formula 1.873).

Table 874. Compounds of formula I.A, wherein R² is R²-10.188(=formula 1.874).

Table 875. Compounds of formula I.A, wherein R² is R²-10.189(=formula 1.875).

Table 876. Compounds of formula I.A, wherein R² is R²-10.190(=formula 1.876).

Table 877. Compounds of formula I.A, wherein R² is R²-10.191(=formula 1.877).

Table 878. Compounds of formula I.A, wherein R² is R²-10.192(=formula 1.878).

Table 879. Compounds of formula I.A, wherein R² is R²-10.193(=formula 1.879).

Table 880. Compounds of formula I.A, wherein R² is R²-10.194(=formula 1.880).

Table 881. Compounds of formula I.A, wherein R² is R²-10.195(=formula 1.881).

Table 882. Compounds of formula I.A, wherein R² is R²-10.196(=formula 1.882).

Table 883. Compounds of formula I.A, wherein R² is R²-10.197(=formula 1.883).

Table 884. Compounds of formula I.A, wherein R² is R²-10.198(=formula 1.884).

Table 885. Compounds of formula I.A, wherein R² is R²-10.199(=formula 1.885).

Table 886. Compounds of formula I.A, wherein R² is R²-10.200(=formula 1.886).

Table 887. Compounds of formula I.A, wherein R² is R²-10.201(=formula 1.887).

Table 888. Compounds of formula I.A, wherein R² is R²-10.202(=formula 1.888).

Table 889. Compounds of formula I.A, wherein R² is R²-10.203(=formula 1.889).

Table 890. Compounds of formula I.A, wherein R² is R²-10.204(=formula 1.890).

Table 891. Compounds of formula I.A, wherein R² is R²-10.205(=formula 1.891).

Table 892. Compounds of formula I.A, wherein R² is R²-10.206(=formula 1.892).

Table 893. Compounds of formula I.A, wherein R² is R²-10.207(=formula 1.893).

Table 894. Compounds of formula I.A, wherein R² is R²-10.208(=formula 1.894).

Table 895. Compounds of formula I.A, wherein R² is R²-10.209(=formula 1.895).

Table 896. Compounds of formula I.A, wherein R² is R²-10.210(=formula 1.896).

Table 897. Compounds of formula I.A, wherein R² is R²-10.211(=formula 1.897).

Table 898. Compounds of formula I.A, wherein R² is R²-10.212(=formula 1.898).

Table 899. Compounds of formula I.A, wherein R² is R²-10.213(=formula 1.899).

Table 900. Compounds of formula I.A, wherein R² is R²-10.214(=formula 1.900).

Table 901. Compounds of formula I.A, wherein R² is R²-10.215(=formula 1.901).

Table 902. Compounds of formula I.A, wherein R² is R²-10.216(=formula 1.902).

Table 903. Compounds of formula I.A, wherein R² is R²-10.217(=formula 1.903).

Table 904. Compounds of formula I.A, wherein R² is R²-10.218(=formula 1.904).

Table 905. Compounds of formula I.A, wherein R² is R²-10.219(=formula 1.905).

Table 906. Compounds of formula I.A, wherein R² is R²-10.220(=formula 1.906).

Table 907. Compounds of formula I.A, wherein R² is R²-10.221(=formula 1.907).

Table 908. Compounds of formula I.A, wherein R² is R²-10.222(=formula 1.908).

Table 909. Compounds of formula I.A, wherein R² is R²-10.223(=formula 1.909).

Table 910. Compounds of formula I.A, wherein R² is R²-10.224(=formula 1.910).

Table 911. Compounds of formula I.A, wherein R² is R²-10.225(=formula 1.911).

Table 912. Compounds of formula I.A, wherein R² is R²-10.226(=formula 1.912).

Table 913. Compounds of formula I.A, wherein R² is R²-10.227(=formula 1.913).

Table 914. Compounds of formula I.A, wherein R² is R²-10.228(=formula 1.914).

Table 915. Compounds of formula I.A, wherein R² is R²-10.229(=formula 1.915).

Table 916. Compounds of formula I.A, wherein R² is R²-10.230(=formula 1.916).

Table 917. Compounds of formula I.A, wherein R² is R²-10.231(=formula 1.917).

Table 918. Compounds of formula I.A, wherein R² is R²-10.232(=formula 1.918).

Table 919. Compounds of formula I.A, wherein R² is R²-10.233(=formula 1.919).

Table 920. Compounds of formula I.A, wherein R² is R²-10.234(=formula 1.920).

Table 921. Compounds of formula I.A, wherein R² is R²-10.235(=formula 1.921).

Table 922. Compounds of formula I.A, wherein R² is R²-10.236(=formula 1.922).

Table 923. Compounds of formula I.A, wherein R² is R²-10.237(=formula 1.923).

Table 924. Compounds of formula I.A, wherein R² is R²-10.238(=formula 1.924).

Table 925. Compounds of formula I.A, wherein R² is R²-10.239(=formula 1.925).

Table 926. Compounds of formula I.A, wherein R² is R²-10.240(=formula 1.926).

Table 927. Compounds of formula I.A, wherein R² is R²-10.241(=formula 1.927).

Table 928. Compounds of formula I.A, wherein R² is R²-10.242(=formula 1.928).

Table 929. Compounds of formula I.A, wherein R² is R²-10.243(=formula 1.929).

Table 930. Compounds of formula I.A, wherein R² is R²-10.244(=formula 1.930).

Table 931. Compounds of formula I.A, wherein R² is R²-10.245(=formula 1.931).

Table 932. Compounds of formula I.A, wherein R² is R²-10.246(=formula 1.932).

Table 933. Compounds of formula I.A, wherein R² is R²-10.247(=formula 1.933).

Table 934. Compounds of formula I.A, wherein R² is R²-10.248(=formula 1.934).

Table 935. Compounds of formula I.A, wherein R² is R²-10.249(=formula 1.935).

Table 936. Compounds of formula I.A, wherein R² is R²-10.250(=formula 1.936).

Table 937. Compounds of formula I.A, wherein R² is R²-10.251(=formula 1.937).

Table 938. Compounds of formula I.A, wherein R² is R²-10.252(=formula 1.938).

Table 939. Compounds of formula I.A, wherein R² is R²-10.253(=formula 1.939).

Table 940. Compounds of formula I.A, wherein R² is R²-10.254(=formula 1.940).

Table 941. Compounds of formula I.A, wherein R² is R²-10.255(=formula 1.941).

Table 942. Compounds of formula I.A, wherein R² is R²-10.256(=formula 1.942).

Table 943. Compounds of formula I.A, wherein R² is R²-10.257(=formula 1.943).

Table 944. Compounds of formula I.A, wherein R² is R²-10.258(=formula 1.944).

Table 945. Compounds of formula I.A, wherein R² is R²-10.259(=formula 1.945).

Table 946. Compounds of formula I.A, wherein R² is R²-10.260(=formula 1.946).

Table 947. Compounds of formula I.A, wherein R² is R²-10.261(=formula 1.947).

Table 948. Compounds of formula I.A, wherein R² is R²-10.262(=formula 1.948).

Table 949. Compounds of formula I.A, wherein R² is R²-10.263(=formula 1.949).

Table 950. Compounds of formula I.A, wherein R² is R²-10.264(=formula 1.950).

Table 951. Compounds of formula I.A, wherein R² is R²-10.265(=formula 1.951).

Table 952. Compounds of formula I.A, wherein R² is R²-10.266(=formula 1.952).

Table 953. Compounds of formula I.A, wherein R² is R²-10.267(=formula 1.953).

Table 954. Compounds of formula I.A, wherein R² is R²-10.268(=formula 1.954).

Table 955. Compounds of formula I.A, wherein R² is R²-10.269(=formula 1.955).

Table 956. Compounds of formula I.A, wherein R² is R²-10.270(=formula 1.956).

Table 957. Compounds of formula I.A, wherein R² is R²-10.271(=formula 1.957).

Table 958. Compounds of formula I.A, wherein R² is R²-10.272(=formula 1.958).

Table 959. Compounds of formula I.A, wherein R² is R²-10.273(=formula 1.959).

Table 960. Compounds of formula I.A, wherein R² is R²-10.274(=formula 1.960).

Table 961. Compounds of formula I.A, wherein R² is R²-10.275(=formula 1.961).

Table 962. Compounds of formula I.A, wherein R² is R²-10.276(=formula 1.962).

Table 963. Compounds of formula I.A, wherein R² is R²-10.277(=formula 1.963).

Table 964. Compounds of formula I.A, wherein R² is R²-10.278(=formula 1.964).

Table 965. Compounds of formula I.A, wherein R² is R²-10.279(=formula 1.965).

Table 966. Compounds of formula I.A, wherein R² is R²-10.280(=formula 1.966).

Table 967. Compounds of formula I.A, wherein R² is R²-10.281(=formula 1.967).

Table 968. Compounds of formula I.A, wherein R² is R²-10.282(=formula 1.968).

Table 969. Compounds of formula I.A, wherein R² is R²-10.283(=formula 1.969).

Table 970. Compounds of formula I.A, wherein R² is R²-10.284(=formula 1.970).

Table 971. Compounds of formula I.A, wherein R² is R²-10.285(=formula 1.971).

Table 972. Compounds of formula I.A, wherein R² is R²-10.286(=formula 1.972).

Table 973. Compounds of formula I.A, wherein R² is R²-10.287(=formula 1.973).

Table 974. Compounds of formula I.A, wherein R² is R²-10.288(=formula 1.974).

Table 975. Compounds of formula I.A, wherein R² is R²-10.289(=formula 1.975).

Table 976. Compounds of formula I.A, wherein R² is R²-10.290(=formula 1.976).

Table 977. Compounds of formula I.A, wherein R² is R²-10.291(=formula 1.977).

Table 978. Compounds of formula I.A, wherein R² is R²-10.292(=formula 1.978).

Table 979. Compounds of formula I.A, wherein R² is R²-10.293(=formula 1.979).

Table 980. Compounds of formula I.A, wherein R² is R²-10.294(=formula 1.980).

Table 981. Compounds of formula I.A, wherein R² is R²-10.295(=formula 1.981).

Table 982. Compounds of formula I.A, wherein R² is R²-10.296(=formula 1.982).

Table 983. Compounds of formula I.A, wherein R² is R²-10.297(=formula 1.983).

Table 984. Compounds of formula I.A, wherein R² is R²-10.298(=formula 1.984).

Table 985. Compounds of formula I.A, wherein R² is R²-10.299(=formula 1.985).

Table 986. Compounds of formula I.A, wherein R² is R²-10.300(=formula 1.986).

Table 987. Compounds of formula I.A, wherein R² is R²-10.301(=formula 1.987).

Table 988. Compounds of formula I.A, wherein R² is R²-10.302(=formula 1.988).

Table 989. Compounds of formula I.A, wherein R² is R²-10.303(=formula 1.989).

Table 990. Compounds of formula I.A, wherein R² is R²-10.304(=formula 1.990).

Table 991. Compounds of formula I.A, wherein R² is R²-10.305(=formula 1.991).

Table 992. Compounds of formula I.A, wherein R² is R²-10.306(=formula 1.992).

Table 993. Compounds of formula I.A, wherein R² is R²-10.307(=formula 1.993).

Table 994. Compounds of formula I.A, wherein R² is R²-10.308(=formula 1.994).

Table 995. Compounds of formula I.A, wherein R² is R²-10.309(=formula 1.995).

Table 996. Compounds of formula I.A, wherein R² is R²-10.310(=formula 1.996).

Table 997. Compounds of formula I.A, wherein R² is R²-10.311(=formula 1.997).

Table 998. Compounds of formula I.A, wherein R² is R²-10.312(=formula 1.998).

Table 999. Compounds of formula I.A, wherein R² is R²-10.313(=formula 1.999).

Table 1000. Compounds of formula I.A, wherein R² is R²-10.314(=formula 1.1000).

Table 1001. Compounds of formula I.A, wherein R² is R²-10.315(=formula 1.1001).

Table 1002. Compounds of formula I.A, wherein R² is R²-10.316(=formula 1.1002).

Table 1003. Compounds of formula I.A, wherein R² is R²-10.317(=formula 1.1003).

Table 1004. Compounds of formula I.A, wherein R² is R²-10.318(=formula 1.1004).

Table 1005. Compounds of formula I.A, wherein R² is R²-10.319(=formula 1.1005).

Table 1006. Compounds of formula I.A, wherein R² is R²-10.320(=formula 1.1006).

Table 1007. Compounds of formula I.A, wherein R² is R²-10.321(=formula 1.1007).

Table 1008. Compounds of formula I.A, wherein R² is R²-10.322(=formula 1.1008).

Table 1009. Compounds of formula I.A, wherein R² is R²-10.323(=formula 1.1009).

Table 1010. Compounds of formula I.A, wherein R² is R²-10.324(=formula 1.1010).

Table 1011. Compounds of formula I.A, wherein R² is R²-10.325(=formula 1.1011).

Table 1012. Compounds of formula I.A, wherein R² is R²-10.326(=formula 1.1012).

Table 1013. Compounds of formula I.A, wherein R² is R²-10.327(=formula 1.1013).

Table 1014. Compounds of formula I.A, wherein R² is R²-10.328(=formula 1.1014).

Table 1015. Compounds of formula I.A, wherein R² is R²-10.329(=formula 1.1015).

Table 1016. Compounds of formula I.A, wherein R² is R²-10.330(=formula 1.1016).

Table 1017. Compounds of formula I.A, wherein R² is R²-10.331(=formula 1.1017).

Table 1018. Compounds of formula I.A, wherein R² is R²-10.332(=formula 1.1018).

Table 1019. Compounds of formula I.A, wherein R² is R²-10.333(=formula 1.1019).

Table 1020. Compounds of formula I.A, wherein R² is R²-10.334(=formula 1.1020).

Table 1021. Compounds of formula I.A, wherein R² is R²-10.335(=formula 1.1021).

Table 1022. Compounds of formula I.A, wherein R² is R²-10.336(=formula 1.1022).

Table 1023. Compounds of formula I.A, wherein R² is R²-10.334(=formula 1.1023).

Table 1024. Compounds of formula I.A, wherein R² is R²-10.338(=formula 1.1024).

Table 1025. Compounds of formula I.A, wherein R² is R²-10.339(=formula 1.1025).

Table 1026. Compounds of formula I.A, wherein R² is R²-10.340(=formula 1.1026).

Table 1027. Compounds of formula I.A, wherein R² is R²-10.341(=formula 1.1027).

Table 1028. Compounds of formula I.A, wherein R² is R²-10.342(=formula 1.1028).

Table 1029. Compounds of formula I.A, wherein R² is R²-10.343(=formula 1.1029).

Table 1030. Compounds of formula I.A, wherein R² is R²-10.344(=formula 1.1030).

Table 1031. Compounds of formula I.A, wherein R² is R²-10.345(=formula 1.1031).

Table 1032. Compounds of formula I.A, wherein R² is R²-10.346(=formula 1.1032).

Table 1033. Compounds of formula I.A, wherein R² is R²-10.347(=formula 1.1033).

Table 1034. Compounds of formula I.A, wherein R² is R²-10.348(=formula 1.1034).

Table 1035. Compounds of formula I.A, wherein R² is R²-10.349(=formula 1.1035).

Table 1036. Compounds of formula I.A, wherein R² is R²-10.350(=formula 1.1036).

Table 1037. Compounds of formula I.A, wherein R² is R²-10.351(=formula 1.1037).

Table 1038. Compounds of formula I.A, wherein R² is R²-10.352(=formula 1.1038).

Table 1039. Compounds of formula I.A, wherein R² is R²-10.353(=formula 1.1039).

Table 1040. Compounds of formula I.A, wherein R² is R²-10.354(=formula 1.1040).

Table 1041. Compounds of formula I.A, wherein R² is R²-10.355(=formula 1.1041).

Table 1042. Compounds of formula I.A, wherein R² is R²-10.356(=formula 1.1042).

Table 1043. Compounds of formula I.A, wherein R² is R²-10.357(=formula 1.1043).

Table 1044. Compounds of formula I.A, wherein R² is R²-10.358(=formula 1.1044).

Table 1045. Compounds of formula I.A, wherein R² is R²-10.359(=formula 1.1045).

Table 1046. Compounds of formula I.A, wherein R² is R²-10.360(=formula 1.1046).

Table 1047. Compounds of formula I.A, wherein R² is R²-10.361(=formula 1.1047).

Table 1048. Compounds of formula I.A, wherein R² is R²-10.362(=formula 1.1048).

Table 1049. Compounds of formula I.A, wherein R² is R²-10.363(=formula 1.1049).

Table 1050. Compounds of formula I.A, wherein R² is R²-10.364(=formula 1.1050).

Table 1051. Compounds of formula I.A, wherein R² is R²-10.365(=formula 1.1051).

Table 1052. Compounds of formula I.A, wherein R² is R²-10.366(=formula 1.1052).

Table 1053. Compounds of formula I.A, wherein R² is R²-10.367(=formula 1.1053).

Table 1054. Compounds of formula I.A, wherein R² is R²-10.368(=formula 1.1054).

Table 1055. Compounds of formula I.A, wherein R² is R²-10.369(=formula 1.1055).

Table 1056. Compounds of formula I.A, wherein R² is R²-10.370(=formula 1.1056).

Table 1057. Compounds of formula I.A, wherein R² is R²-10.371(=formula 1.1057).

Table 1058. Compounds of formula I.A, wherein R² is R²-10.372(=formula 1.1058).

Table 1059. Compounds of formula I.A, wherein R² is R²-10.373(=formula 1.1059).

Table 1060. Compounds of formula I.A, wherein R² is R²-10.374(=formula 1.1060).

Table 1061. Compounds of formula I.A, wherein R² is R²-10.375(=formula 1.1061).

Table 1062. Compounds of formula I.A, wherein R² is R²-10.376(=formula 1.1062).

Table 1063. Compounds of formula I.A, wherein R² is R²-10.377(=formula 1.1063).

Table 1064. Compounds of formula I.A, wherein R² is R²-10.378(=formula 1.1064).

Table 1065. Compounds of formula I.A, wherein R² is R²-10.379(=formula 1.1065).

Table 1066. Compounds of formula I.A, wherein R² is R²-10.380(=formula 1.1066).

Table 1067. Compounds of formula I.A, wherein R² is R²-10.381(=formula 1.1067).

Table 1068. Compounds of formula I.A, wherein R² is R²-10.382(=formula 1.1068).

Table 1069. Compounds of formula I.A, wherein R² is R²-10.383(=formula 1.1069).

Table 1070. Compounds of formula I.A, wherein R² is R²-10.384(=formula 1.1070).

Table 1071. Compounds of formula I.A, wherein R² is R²-10.385(=formula 1.1071).

Table 1072. Compounds of formula I.A, wherein R² is R²-10.386(=formula 1.1072).

Table 1073. Compounds of formula I.A, wherein R² is R²-10.387(=formula 1.1073).

Table 1074. Compounds of formula I.A, wherein R² is R²-10.388(=formula 1.1074).

Table 1075. Compounds of formula I.A, wherein R² is R²-10.389(=formula 1.1075).

Table 1076. Compounds of formula I.A, wherein R² is R²-10.390(=formula 1.7076).

Table 1077. Compounds of formula I.A, wherein R² is R²-10.391(=formula 1.1077).

Table 1078. Compounds of formula I.A, wherein R² is R²-10.392(=formula 1.1078).

Table 1079. Compounds of formula I.A, wherein R² is R²-10.393(=formula 1.1079).

Table 1080. Compounds of formula I.A, wherein R² is R²-10.394(=formula 1.1080).

Table 1081. Compounds of formula I.A, wherein R² is R²-10.395(=formula 1.1081).

Table 1082. Compounds of formula I.A, wherein R² is R²-10.396(=formula 1.1082).

Table 1083. Compounds of formula I.A, wherein R² is R²-10.397(=formula 1.1083).

Table 1084. Compounds of formula I.A, wherein R² is R²-10.398(=formula 1.1084).

Table 1085. Compounds of formula I.A, wherein R² is R²-10.399(=formula 1.1085).

Table 1086. Compounds of formula I.A, wherein R² is R²-10.400(=formula 1.1086).

Table 1087. Compounds of formula I.A, wherein R² is R²-10.401(=formula 1.1087).

Table 1088. Compounds of formula I.A, wherein R² is R²-10.402(=formula 1.1088).

Table 1089. Compounds of formula I.A, wherein R² is R²-10.403(=formula 1.1089).

Table 1090. Compounds of formula I.A, wherein R² is R²-10.404(=formula 1.1090).

Table 1091. Compounds of formula I.A, wherein R² is R²-10.405(=formula 1.1091).

Table 1092. Compounds of formula I.A, wherein R² is R²-10.406(=formula 1.1092).

Table 1093. Compounds of formula I.A, wherein R² is R²-10.407(=formula 1.1093).

Table 1094. Compounds of formula I.A, wherein R² is R²-10.408(=formula 1.1094).

Table 1095. Compounds of formula I.A, wherein R² is R²-10.409(=formula 1.1095).

Table 1096. Compounds of formula I.A, wherein R² is R²-10.410(=formula 1.1096).

Table 1097. Compounds of formula I.A, wherein R² is R²-10.411(=formula 1.1097).

Table 1098. Compounds of formula I.A, wherein R² is R²-10.412(=formula 1.1098).

Table 1099. Compounds of formula I.A, wherein R² is R²-10.413(=formula 1.1099).

Table 1100. Compounds of formula I.A, wherein R² is R²-10.414(=formula 1.1100).

Table 1101. Compounds of formula I.A, wherein R² is R²-10.415(=formula 1.1101).

Table 1102. Compounds of formula I.A, wherein R² is R²-10.416(=formula 1.1102).

Table 1103. Compounds of formula I.A, wherein R² is R²-10.417(=formula 1.1103).

Table 1104. Compounds of formula I.A, wherein R² is R²-10.418(=formula 1.1104).

Table 1105. Compounds of formula I.A, wherein R² is R²-10.419(=formula 1.1105).

Table 1106. Compounds of formula I.A, wherein R² is R²-10.420(=formula 1.1106).

Table 1107. Compounds of formula I.A, wherein R² is R²-10.421(=formula 1.1107).

Table 1108. Compounds of formula I.A, wherein R² is R²-10.422(=formula 1.1108).

Table 1109. Compounds of formula I.A, wherein R² is R²-10.423(=formula 1.1109).

Table 1110. Compounds of formula I.A, wherein R² is R²-10.424(=formula 1.1110).

Table 1111. Compounds of formula I.A, wherein R² is R²-10.425(=formula 1.1111).

Table 1112. Compounds of formula I.A, wherein R² is R²-10.426(=formula 1.1112).

Table 1113. Compounds of formula I.A, wherein R² is R²-10.427(=formula 1.1113).

Table 1114. Compounds of formula I.A, wherein R² is R²-10.428(=formula 1.1114).

Table 1115. Compounds of formula I.A, wherein R² is R²-10.429(=formula 1.1115).

Table 1116. Compounds of formula I.A, wherein R² is R²-10.430(=formula 1.1116).

Table 1117. Compounds of formula I.A, wherein R² is R²-10.431(=formula 1.1117).

Table 1118. Compounds of formula I.A, wherein R² is R²-10.432(=formula 1.1118).

Table 1119. Compounds of formula I.A, wherein R² is R²-10.433(=formula 1.1119).

Table 1120. Compounds of formula I.A, wherein R² is R²-10.434(=formula 1.1120).

Table 1121. Compounds of formula I.A, wherein R² is R²-10.435(=formula 1.1121).

Table 1122. Compounds of formula I.A, wherein R² is R²-10.436(=formula 1.1122).

Table 1123. Compounds of formula I.A, wherein R² is R²-10.437(=formula 1.1123).

Table 1124. Compounds of formula I.A, wherein R² is R²-10.438(=formula 1.1124).

Table 1125. Compounds of formula I.A, wherein R² is R²-10.439(=formula 1.1125).

Table 1126. Compounds of formula I.A, wherein R² is R²-10.440(=formula 1.1126).

Table 1127. Compounds of formula I.A, wherein R² is R²-10.441(=formula 1.1127).

Table 1128. Compounds of formula I.A, wherein R² is R²-10.442(=formula 1.1128).

Table 1129. Compounds of formula I.A, wherein R² is R²-10.443(=formula 1.1129).

Table 1130. Compounds of formula I.A, wherein R² is R²-10.444(=formula 1.1130).

Table 1131. Compounds of formula I.A, wherein R² is R²-10.445(=formula 1.1131).

Table 1132. Compounds of formula I.A, wherein R² is R²-10.446(=formula 1.1132).

Table 1133. Compounds of formula I.A, wherein R² is R²-10.447(=formula 1.1133).

Table 1134. Compounds of formula I.A, wherein R² is R²-10.448(=formula 1.1134).

Table 1135. Compounds of formula I.A, wherein R² is R²-10.449(=formula 1.1135).

Table 1136. Compounds of formula I.A, wherein R² is R²-10.450(=formula 1.1136).

Table 1137. Compounds of formula I.A, wherein R² is R²-10.451(=formula 1.1137).

Table 1138. Compounds of formula I.A, wherein R² is R²-10.452(=formula 1.1138).

Table 1139. Compounds of formula I.A, wherein R² is R²-10.453(=formula 1.1139).

Table 1140. Compounds of formula I.A, wherein R² is R²-10.454(=formula 1.1140).

Table 1141. Compounds of formula I.A, wherein R² is R²-10.455(=formula 1.1141).

Table 1142. Compounds of formula I.A, wherein R² is R²-10.456(=formula 1.1142).

Table 1143. Compounds of formula I.A, wherein R² is R²-10.457(=formula 1.1143).

Table 1144. Compounds of formula I.A, wherein R² is R²-10.458(=formula 1.1144).

Table 1145. Compounds of formula I.A, wherein R² is R²-10.459(=formula 1.1145).

Table 1146. Compounds of formula I.A, wherein R² is R²-10.460(=formula 1.1146).

Table 1147. Compounds of formula I.A, wherein R² is R²-10.461(=formula 1.1147).

Table 1148. Compounds of formula I.A, wherein R² is R²-10.462(=formula 1.1148).

Table 1149. Compounds of formula I.A, wherein R² is R²-10.463(=formula 1.1149).

Table 1150. Compounds of formula I.A, wherein R² is R²-10.464(=formula 1.1150).

Table 1151. Compounds of formula I.A, wherein R² is R²-10.465(=formula 1.1151).

Table 1152. Compounds of formula I.A, wherein R² is R²-10.466(=formula 1.1152).

Table 1153. Compounds of formula I.A, wherein R² is R²-10.467(=formula 1.1153).

Table 1154. Compounds of formula I.A, wherein R² is R²-10.468(=formula 1.1154).

Table 1155. Compounds of formula I.A, wherein R² is R²-10.469(=formula 1.1154).

Table 1156. Compounds of formula I.A, wherein R² is R²-10.470(=formula 1.1156).

Table 1157. Compounds of formula I.A, wherein R² is R²-10.471(=formula 1.1157).

Table 1158. Compounds of formula I.A, wherein R² is R²-10.472(=formula 1.1158).

Table 1159. Compounds of formula I.A, wherein R² is R²-10.473(=formula 1.1159).

Table 1160. Compounds of formula I.A, wherein R² is R²-10.474(=formula 1.1160).

Table 1161. Compounds of formula I.A, wherein R² is R²-10.475(=formula 1.1161).

Table 1162. Compounds of formula I.A, wherein R² is R²-10.476(=formula 1.1162).

Table 1163. Compounds of formula I.A, wherein R² is R²-10.477(=formula 1.1163).

Table 1164. Compounds of formula I.A, wherein R² is R²-10.478(=formula 1.1164).

Table 1165. Compounds of formula I.A, wherein R² is R²-10.479(=formula 1.1165).

Table 1166. Compounds of formula I.A, wherein R² is R²-10.480(=formula 1.1166).

Table 1167. Compounds of formula I.A, wherein R² is R²-10.481(=formula 1.1167).

Table 1168. Compounds of formula I.A, wherein R² is R²-10.482(=formula 1.1168).

Table 1169. Compounds of formula I.A, wherein R² is R²-10.483(=formula 1.1169).

Table 1170. Compounds of formula I.A, wherein R² is R²-10.484(=formula 1.1170).

Table 1171. Compounds of formula I.A, wherein R² is R²-10.485(=formula 1.1171).

Table 1172. Compounds of formula I.A, wherein R² is R²-10.486(=formula 1.1172).

Table 1173. Compounds of formula I.A, wherein R² is R²-10.487(=formula 1.1173).

Table 1174. Compounds of formula I.A, wherein R² is R²-10.488(=formula 1.1174).

Table 1175. Compounds of formula I.A, wherein R² is R²-10.489(=formula 1.1175).

Table 1176. Compounds of formula I.A, wherein R² is R²-10.490(=formula 1.1176).

Table 1177. Compounds of formula I.A, wherein R² is R²-10.491(=formula 1.1177).

Table 1178. Compounds of formula I.A, wherein R² is R²-10.492(=formula 1.1178).

Table 1179. Compounds of formula I.A, wherein R² is R²-10.493(=formula 1.1179).

Table 1180. Compounds of formula I.A, wherein R² is R²-10.494(=formula 1.1180).

Table 1181. Compounds of formula I.A, wherein R² is R²-10.495(=formula 1.1181).

Table 1182. Compounds of formula I.A, wherein R² is R²-10.496(=formula 1.1182).

Table 1183. Compounds of formula I.A, wherein R² is R²-10.497(=formula 1.1183).

Table 1184. Compounds of formula I.A, wherein R² is R²-10.498(=formula 1.1184).

Table 1185. Compounds of formula I.A, wherein R² is R²-10.499(=formula 1.1185).

Table 1186. Compounds of formula I.A, wherein R² is R²-10.500(=formula 1.1186).

Table 1187. Compounds of formula I.A, wherein R² is R²-10.501(=formula 1.1187).

Table 1188. Compounds of formula I.A, wherein R² is R²-10.502(=formula 1.1188).

Table 1189. Compounds of formula I.A, wherein R² is R²-10.503(=formula 1.1189).

Table 1190. Compounds of formula I.A, wherein R² is R²-10.504(=formula 1.1190).

Table 1191. Compounds of formula I.A, wherein R² is R²-10.505(=formula 1.1191).

Table 1192. Compounds of formula I.A, wherein R² is R²-10.506(=formula 1.1192).

Table 1193. Compounds of formula I.A, wherein R² is R²-10.507(=formula 1.1193).

Table 1194. Compounds of formula I.A, wherein R² is R²-10.508(=formula 1.1194).

Table 1195. Compounds of formula I.A, wherein R² is R²-10.509(=formula 1.1195).

Table 1196. Compounds of formula I.A, wherein R² is R²-10.510(=formula 1.1196).

Table 1197. Compounds of formula I.A, wherein R² is R²-10.511(=formula 1.1197).

Table 1198. Compounds of formula I.A, wherein R² is R²-10.512(=formula 1.1198).

Table 1199. Compounds of formula I.A, wherein R² is R²-10.513(=formula 1.1199).

Table 1200. Compounds of formula I.A, wherein R² is R²-10.514(=formula 1.1200).

Table 1201. Compounds of formula I.A, wherein R² is R²-10.515(=formula 1.1201).

Table 1202. Compounds of formula I.A, wherein R² is R²-10.516(=formula 1.1202).

Table 1203. Compounds of formula I.A, wherein R² is R²-10.517(=formula 1.1203).

Table 1204. Compounds of formula I.A, wherein R² is R²-10.518(=formula 1.1204).

Table 1205. Compounds of formula I.A, wherein R² is R²-10.519(=formula 1.1205).

Table 1206. Compounds of formula I.A, wherein R² is R²-10.520(=formula 1.1206).

Table 1207. Compounds of formula I.A, wherein R² is R²-10.521(=formula 1.1207).

Table 1208. Compounds of formula I.A, wherein R² is R²-10.522(=formula 1.1208).

Table 1209. Compounds of formula I.A, wherein R² is R²-10.523(=formula 1.1209).

Table 1210. Compounds of formula I.A, wherein R² is R²-10.524(=formula 1.1210).

Table 1211. Compounds of formula I.A, wherein R² is R²-10.525(=formula 1.1211).

Table 1212. Compounds of formula I.A, wherein R² is R²-10.526(=formula 1.1212).

Table 1213. Compounds of formula I.A, wherein R² is R²-10.527(=formula 1.1213).

Table 1214. Compounds of formula I.A, wherein R² is R²-10.528(=formula 1.1212).

Table 1215. Compounds of formula I.A, wherein R² is R²-10.529(=formula 1.1215).

Table 1216. Compounds of formula I.A, wherein R² is R²-10.530(=formula 1.1216).

Table 1217. Compounds of formula I.A, wherein R² is R²-10.531(=formula 1.1217).

Table 1218. Compounds of formula I.A, wherein R² is R²-10.532(=formula 1.1218).

Table 1219. Compounds of formula I.A, wherein R² is R²-10.533(=formula 1.1219).

Table 1220. Compounds of formula I.A, wherein R² is R²-10.534(=formula 1.1220).

Table 1221. Compounds of formula I.A, wherein R² is R²-10.535(=formula 1.1221).

Table 1222. Compounds of formula I.A, wherein R² is R²-10.536(=formula 1.1222).

Table 1223. Compounds of formula I.A, wherein R² is R²-10.537(=formula 1.1223).

Table 1224. Compounds of formula I.A, wherein R² is R²-10.538(=formula 1.1224).

Table 1225. Compounds of formula I.A, wherein R² is R²-10.539(=formula 1.1225).

Table 1226. Compounds of formula I.A, wherein R² is R²-10.540(=formula 1.1226).

Table 1227. Compounds of formula I.A, wherein R² is R²-10.541(=formula 1.1227).

Table 1228. Compounds of formula I.A, wherein R² is R²-10.542(=formula 1.1228).

Table 1229. Compounds of formula I.A, wherein R² is R²-10.543(=formula 1.1229).

Table 1230. Compounds of formula I.A, wherein R² is R²-10.544(=formula 1.1230).

Table 1231. Compounds of formula I.A, wherein R² is R²-10.545(=formula 1.1231).

Table 1232. Compounds of formula I.A, wherein R² is R²-10.546(=formula 1.1232).

Table 1233. Compounds of formula I.A, wherein R² is R²-10.547(=formula 1.1233).

Table 1234. Compounds of formula I.A, wherein R² is R²-10.548(=formula 1.1234).

Table 1235. Compounds of formula I.A, wherein R² is R²-10.549(=formula 1.1235).

Table 1236. Compounds of formula I.A, wherein R² is R²-10.550(=formula 1.1236).

Table 1237. Compounds of formula I.A, wherein R² is R²-10.551(=formula 1.1237).

Table 1238. Compounds of formula I.A, wherein R² is R²-10.552(=formula 1.1238).

Table 1239. Compounds of formula I.A, wherein R² is R²-10.553(=formula 1.1239).

Table 1240. Compounds of formula I.A, wherein R² is R²-10.554(=formula 1.1240).

Table 1241. Compounds of formula I.A, wherein R² is R²-10.555(=formula 1.1241).

Table 1242. Compounds of formula I.A, wherein R² is R²-10.556(=formula 1.1242).

Table 1243. Compounds of formula I.A, wherein R² is R²-10.557(=formula 1.1243).

Table 1244. Compounds of formula I.A, wherein R² is R²-10.558(=formula 1.1244).

Table 1245. Compounds of formula I.A, wherein R² is R²-10.559(=formula 1.1245).

Table 1246. Compounds of formula I.A, wherein R² is R²-10.560(=formula 1.1246).

Table 1247. Compounds of formula I.A, wherein R² is R²-10.561(=formula 1.1247).

Table 1248. Compounds of formula I.A, wherein R² is R²-10.562(=formula 1.1248).

Table 1249. Compounds of formula I.A, wherein R² is R²-10.563(=formula 1.1249).

Table 1250. Compounds of formula I.A, wherein R² is R²-10.564(=formula 1.1350).

Table 1251. Compounds of formula I.A, wherein R² is R²-10.565(=formula 1.1251).

Table 1252. Compounds of formula I.A, wherein R² is R²-10.566(=formula 1.1252).

Table 1253. Compounds of formula I.A, wherein R² is R²-10.567(=formula 1.1253).

Table 1254. Compounds of formula I.A, wherein R² is R²-10.568(=formula 1.1254).

Table 1255. Compounds of formula I.A, wherein R² is R²-10.569(=formula 1.1255).

Table 1256. Compounds of formula I.A, wherein R² is R²-10.570(=formula 1.1256).

Table 1257. Compounds of formula I.A, wherein R² is R²-10.571(=formula 1.1257).

Table 1258. Compounds of formula I.A, wherein R² is R²-10.572(=formula 1.1258).

Table 1259. Compounds of formula I.A, wherein R² is R²-10.573(=formula 1.1259).

Table 1260. Compounds of formula I.A, wherein R² is R²-10.574(=formula 1.1260).

Table 1261. Compounds of formula I.A, wherein R² is R²-10.575(=formula 1.1261).

Table 1262. Compounds of formula I.A, wherein R² is R²-10.576(=formula 1.1262).

Table 1263. Compounds of formula I.A, wherein R² is R²-10.577(=formula 1.1263).

Table 1264. Compounds of formula I.A, wherein R² is R²-10.578(=formula 1.1264).

Table 1265. Compounds of formula I.A, wherein R² is R²-10.579(=formula 1.1265).

Table 1266. Compounds of formula I.A, wherein R² is R²-10.580(=formula 1.1266).

Table 1267. Compounds of formula I.A, wherein R² is R²-10.581(=formula 1.1267).

Table 1268. Compounds of formula I.A, wherein R² is R²-10.582(=formula 1.1268).

Table 1269. Compounds of formula I.A, wherein R² is R²-10.583(=formula 1.1269).

Table 1270. Compounds of formula I.A, wherein R² is R²-10.584(=formula 1.1270).

Table 1271. Compounds of formula I.A, wherein R² is R²-10.585(=formula 1.1271).

Table 1272. Compounds of formula I.A, wherein R² is R²-10.586(=formula 1.1272).

Table 1273. Compounds of formula I.A, wherein R² is R²-10.587(=formula 1.1273).

Table 1274. Compounds of formula I.A, wherein R² is R²-10.588(=formula 1.1274).

Table 1275. Compounds of formula I.A, wherein R² is R²-10.589(=formula 1.1275).

Table 1276. Compounds of formula I.A, wherein R² is R²-10.590(=formula 1.1276).

Table 1277. Compounds of formula I.A, wherein R² is R²-10.591(=formula 1.1277).

Table 1278. Compounds of formula I.A, wherein R² is R²-10.592(=formula 1.1278).

Table 1279. Compounds of formula I.A, wherein R² is R²-10.593(=formula 1.1279).

Table 1280. Compounds of formula I.A, wherein R² is R²-10.594(=formula 1.1280).

Table 1281. Compounds of formula I.A, wherein R² is R²-10.595(=formula 1.1281).

Table 1282. Compounds of formula I.A, wherein R² is R²-10.596(=formula 1.1282).

Table 1283. Compounds of formula I.A, wherein R² is R²-10.597(=formula 1.1283).

Table 1284. Compounds of formula I.A, wherein R² is R²-10.598(=formula 1.1284).

Table 1285. Compounds of formula I.A, wherein R² is R²-10.599(=formula 1.1285).

Table 1286. Compounds of formula I.A, wherein R² is R²-10.600(=formula 1.1286).

Table 1287. Compounds of formula I.A, wherein R² is R²-10.601(=formula 1.1287).

Table 1288. Compounds of formula I.A, wherein R² is R²-10.602(=formula 1.1288).

Table 1289. Compounds of formula I.A, wherein R² is R²-10.603(=formula 1.1289).

Table 1290. Compounds of formula I.A, wherein R² is R²-10.604(=formula 1.1290).

Table 1291. Compounds of formula I.A, wherein R² is R²-10.605(=formula 1.1291).

Table 1292. Compounds of formula I.A, wherein R² is R²-10.606(=formula 1.1292).

Table 1293. Compounds of formula I.A, wherein R² is R²-10.607(=formula 1.1293).

Table 1294. Compounds of formula I.A, wherein R² is R²-10.608(=formula 1.1294).

Table 1295. Compounds of formula I.A, wherein R² is R²-10.609(=formula 1.1295).

Table 1296. Compounds of formula I.A, wherein R² is R²-10.610(=formula 1.1296).

Table 1297. Compounds of formula I.A, wherein R² is R²-10.611(=formula 1.1297).

Table 1298. Compounds of formula I.A, wherein R² is R²-10.612(=formula 1.1298).

Table 1299. Compounds of formula I.A, wherein R² is R²-10.613(=formula 1.1299).

Table 1300. Compounds of formula I.A, wherein R² is R²-10.614(=formula 1.1300).

Table 1301. Compounds of formula I.A, wherein R² is R²-10.615(=formula 1.1301).

Table 1302. Compounds of formula I.A, wherein R² is R²-10.616(=formula 1.1302).

Table 1303. Compounds of formula I.A, wherein R² is R²-10.617(=formula 1.1303).

Table 1304. Compounds of formula I.A, wherein R² is R²-10.618(=formula 1.1304).

Table 1305. Compounds of formula I.A, wherein R² is R²-10.619(=formula 1.1305).

Table 1306. Compounds of formula I.A, wherein R² is R²-10.620(=formula 1.1306).

Table 1307. Compounds of formula I.A, wherein R² is R²-10.621(=formula 1.1307).

Table 1308. Compounds of formula I.A, wherein R² is R²-10.622(=formula 1.1308).

Table 1309. Compounds of formula I.A, wherein R² is R²-10.623(=formula 1.1309).

Table 1310. Compounds of formula I.A, wherein R² is R²-10.624(=formula 1.1310).

Table 1311. Compounds of formula I.A, wherein R² is R²-10.625(=formula 1.1311).

Table 1312. Compounds of formula I.A, wherein R² is R²-10.626(=formula 1.1312).

Table 1313. Compounds of formula I.A, wherein R² is R²-10.627(=formula 1.1313).

Table 1314. Compounds of formula I.A, wherein R² is R²-10.628(=formula 1.1314).

Table 1315. Compounds of formula I.A, wherein R² is R²-10.629(=formula 1.1315).

Table 1316. Compounds of formula I.A, wherein R² is R²-10.630(=formula 1.1316).

Table 1317. Compounds of formula I.A, wherein R² is R²-10.631(=formula 1.1317).

Table 1318. Compounds of formula I.A, wherein R² is R²-10.632(=formula 1.1318).

Table 1319. Compounds of formula I.A, wherein R² is R²-10.633(=formula 1.1319).

Table 1320. Compounds of formula I.A, wherein R² is R²-10.634(=formula 1.1320).

Table 1321. Compounds of formula I.A, wherein R² is R²-10.635(=formula 1.1321).

Table 1322. Compounds of formula I.A, wherein R² is R²-10.636(=formula 1.1322).

Table 1323. Compounds of formula I.A, wherein R² is R²-10.637(=formula 1.1323).

Table 1324. Compounds of formula I.A, wherein R² is R²-10.638(=formula 1.1324).

Table 1325. Compounds of formula I.A, wherein R² is R²-10.639(=formula 1.1325).

Table 1326. Compounds of formula I.A, wherein R² is R²-10.640(=formula 1.1326).

Table 1327. Compounds of formula I.A, wherein R² is R²-10.641(=formula 1.1327).

Table 1328. Compounds of formula I.A, wherein R² is R²-10.642(=formula 1.1328).

Table 1329. Compounds of formula I.A, wherein R² is R²-10.643(=formula 1.1329).

Table 1330. Compounds of formula I.A, wherein R² is R²-10.644(=formula 1.1330).

Table 1331. Compounds of formula I.A, wherein R² is R²-10.645(=formula 1.1331).

Table 1332. Compounds of formula I.A, wherein R² is R²-10.646(=formula 1.1332).

Table 1333. Compounds of formula I.A, wherein R² is R²-10.647(=formula 1.1333).

Table 1334. Compounds of formula I.A, wherein R² is R²-10.648(=formula 1.1334).

Table 1335. Compounds of formula I.A, wherein R² is R²-10.649(=formula 1.1335).

Table 1336. Compounds of formula I.A, wherein R² is R²-10.650(=formula 1.1336).

Table 1337. Compounds of formula I.A, wherein R² is R²-10.651(=formula 1.1337).

Table 1338. Compounds of formula I.A, wherein R² is R²-10.652(=formula 1.1338).

Table 1339. Compounds of formula I.A, wherein R² is R²-10.653(=formula 1.1339).

Table 1340. Compounds of formula I.A, wherein R² is R²-10.654(=formula 1.1340).

Table 1341. Compounds of formula I.A, wherein R² is R²-10.655(=formula 1.1341).

Table 1342. Compounds of formula I.A, wherein R² is R²-10.656(=formula 1.1342).

Table 1343. Compounds of formula I.A, wherein R² is R²-10.657(=formula 1.1343).

Table 1344. Compounds of formula I.A, wherein R² is R²-10.658(=formula 1.1344).

Table 1345. Compounds of formula I.A, wherein R² is R²-10.659(=formula 1.1345).

Table 1346. Compounds of formula I.A, wherein R² is R²-10.660(=formula 1.1346).

Table 1347. Compounds of formula I.A, wherein R² is R²-10.661(=formula 1.1347).

Table 1348. Compounds of formula I.A, wherein R² is R²-10.662(=formula 1.1348).

Table 1349. Compounds of formula I.A, wherein R² is R²-10.663(=formula 1.1349).

Table 1350. Compounds of formula I.A, wherein R² is R²-10.664(=formula 1.1350).

Table 1351. Compounds of formula I.A, wherein R² is R²-10.665(=formula 1.1351).

Table 1352. Compounds of formula I.A, wherein R² is R²-10.666(=formula 1.1352).

Table 1353. Compounds of formula I.A, wherein R² is R²-10.667(=formula 1.1353).

Table 1354. Compounds of formula I.A, wherein R² is R²-10.668(=formula 1.1354).

Table 1355. Compounds of formula I.A, wherein R² is R²-10.669(=formula 1.1355).

Table 1356. Compounds of formula I.A, wherein R² is R²-10.670(=formula 1.1356).

Table 1357. Compounds of formula I.A, wherein R² is R²-10.671(=formula 1.1357).

Table 1358. Compounds of formula I.A, wherein R² is R²-10.672(=formula 1.1358).

Table 1359. Compounds of formula I.A, wherein R² is R²-10.673(=formula 1.1359).

Table 1360. Compounds of formula I.A, wherein R² is R²-10.674(=formula 1.1360).

Table 1361. Compounds of formula I.A, wherein R² is R²-10.675(=formula 1.1361).

Table 1362. Compounds of formula I.A, wherein R² is R²-10.676(=formula 1.1362).

Table 1363. Compounds of formula I.A, wherein R² is R²-15.1(=formula 1.1363).

Table 1364. Compounds of formula I.A, wherein R² is R²-15.2(=formula 1.1364).

Table 1365. Compounds of formula I.A, wherein R² is R²-15.3(=formula 1.1365).

Table 1366. Compounds of formula I.A, wherein R² is R²-15.4(=formula 1.1366).

Table 1367. Compounds of formula I.A, wherein R² is R²-15.5(=formula 1.1367).

Table 1368. Compounds of formula I.A, wherein R² is R²-15.6(=formula 1.1368).

Table 1369. Compounds of formula I.A, wherein R² is R²-15.7(=formula 1.1369).

Table 1370. Compounds of formula I.A, wherein R² is R²-15.8(=formula 1.1370).

Table 1371. Compounds of formula I.A, wherein R² is R²-15.9(=formula 1.1371).

Table 1372. Compounds of formula I.A, wherein R² is R²-15.10(=formula 1.1372).

Table 1373. Compounds of formula I.A, wherein R² is R²-15.11(=formula 1.1373).

Table 1374. Compounds of formula I.A, wherein R² is R²-15.12(=formula 1.1374).

Table 1375. Compounds of formula I.A, wherein R² is R²-15.13(=formula 1.1375).

Table 1376. Compounds of formula I.A, wherein R² is R²-15.14(=formula 1.1376).

Table 1377. Compounds of formula I.A, wherein R² is R²-15.15(=formula 1.1377).

Table 1378. Compounds of formula I.A, wherein R² is R²-15.16(=formula 1.1378).

Table 1379. Compounds of formula I.A, wherein R² is R²-15.17(=formula 1.1379).

Table 1380. Compounds of formula I.A, wherein R² is R²-15.18(=formula 1.1380).

Table 1381. Compounds of formula I.A, wherein R² is R²-15.19(=formula 1.1381).

Table 1382. Compounds of formula I.A, wherein R² is R²-15.20(=formula 1.1382).

Table 1383. Compounds of formula I.A, wherein R² is R²-15.21(=formula 1.1383).

Table 1384. Compounds of formula I.A, wherein R² is R²-15.22(=formula 1.1384).

Table 1385. Compounds of formula I.A, wherein R² is R²-15.23(=formula 1.1385).

Table 1386. Compounds of formula I.A, wherein R² is R²-15.24(=formula 1.1386).

Table 1387. Compounds of formula I.A, wherein R² is R²-15.25(=formula 1.1387).

Table 1388. Compounds of formula I.A, wherein R² is R²-15.26(=formula 1.1388).

Table 1389. Compounds of formula I.A, wherein R² is R²-15.27(=formula 1.1389).

Table 1390. Compounds of formula I.A, wherein R² is R²-15.28(=formula 1.1390).

Table 1391. Compounds of formula I.A, wherein R² is R²-15.29(=formula 1.1391).

Table 1392. Compounds of formula I.A, wherein R² is R²-15.30(=formula 1.1392).

Table 1393. Compounds of formula I.A, wherein R² is R²-15.31(=formula 1.1393).

Table 1394. Compounds of formula I.A, wherein R² is R²-15.32(=formula 1.1394).

Table 1395. Compounds of formula I.A, wherein R² is R²-15.33(=formula 1.1395).

Table 1396. Compounds of formula I.A, wherein R² is R²-15.34(=formula 1.1396).

Table 1397. Compounds of formula I.A, wherein R² is R²-15.35(=formula 1.1397).

Table 1398. Compounds of formula I.A, wherein R² is R²-15.36(=formula 1.1398).

Table 1399. Compounds of formula I.A, wherein R² is R²-15.37(=formula 1.1399).

Table 1400. Compounds of formula I.A, wherein R² is R²-15.38(=formula 1.1400).

Table 1401. Compounds of formula I.A, wherein R² is R²-15.39(=formula 1.1401).

Table 1402. Compounds of formula I.A, wherein R² is R²-15.40(=formula 1.1402).

Table 1403. Compounds of formula I.A, wherein R² is R²-15.41(=formula 1.1403).

Table 1404. Compounds of formula I.A, wherein R² is R²-15.42(=formula 1.1404).

Table 1405. Compounds of formula I.A, wherein R² is R²-15.43(=formula 1.1405).

Table 1406. Compounds of formula I.A, wherein R² is R²-15.44(=formula 1.1406).

Table 1407. Compounds of formula I.A, wherein R² is R²-15.45(=formula 1.1407).

Table 1408. Compounds of formula I.A, wherein R² is R²-15.46(=formula 1.1408).

Table 1409. Compounds of formula I.A, wherein R² is R²-15.47(=formula 1.1409).

Table 1410. Compounds of formula I.A, wherein R² is R²-15.48(=formula 1.1410).

Table 1411. Compounds of formula I.A, wherein R² is R²-15.49(=formula 1.1411).

Table 1412. Compounds of formula I.A, wherein R² is R²-15.50(=formula 1.1412).

Table 1413. Compounds of formula I.A, wherein R² is R²-15.51(=formula 1.1413).

Table 1414. Compounds of formula I.A, wherein R² is R²-15.52(=formula 1.1414).

Table 1415. Compounds of formula I.A, wherein R² is R²-15.53(=formula 1.1415).

Table 1416. Compounds of formula I.A, wherein R² is R²-15.54(=formula 1.1416).

Table 1417. Compounds of formula I.A, wherein R² is R²-15.55(=formula 1.1417).

Table 1418. Compounds of formula I.A, wherein R² is R²-15.56(=formula 1.1418).

Table 1419. Compounds of formula I.A, wherein R² is R²-15.57(=formula 1.1419).

Table 1420. Compounds of formula I.A, wherein R² is R²-15.58(=formula 1.1420).

Table 1421. Compounds of formula I.A, wherein R² is R²-15.59(=formula 1.1421).

Table 1422. Compounds of formula I.A, wherein R² is R²-15.60(=formula 1.1422).

Table 1423. Compounds of formula I.A, wherein R² is R²-15.61(=formula 1.1423).

Table 1424. Compounds of formula I.A, wherein R² is R²-15.62(=formula 1.1424).

Table 1425. Compounds of formula I.A, wherein R² is R²-15.63(=formula 1.1425).

Table 1426. Compounds of formula I.A, wherein R² is R²-15.64(=formula 1.1426).

Table 1427. Compounds of formula I.A, wherein R² is R²-15.65(=formula 1.1427).

Table 1428. Compounds of formula I.A, wherein R² is R²-15.66(=formula 1.1428).

Table 1429. Compounds of formula I.A, wherein R² is R²-15.67(=formula 1.1429).

Table 1430. Compounds of formula I.A, wherein R² is R²-15.68(=formula 1.1430).

Table 1431. Compounds of formula I.A, wherein R² is R²-15.69(=formula 1.1431).

Table 1432. Compounds of formula I.A, wherein R² is R²-15.70(=formula 1.1432).

Table 1433. Compounds of formula I.A, wherein R² is R²-15.71(=formula 1.1433).

Table 1434. Compounds of formula I.A, wherein R² is R²-15.72(=formula 1.1434).

Table 1435. Compounds of formula I.A, wherein R² is R²-15.73(=formula 1.1435).

Table 1436. Compounds of formula I.A, wherein R² is R²-15.74(=formula 1.1436).

Table 1437. Compounds of formula I.A, wherein R² is R²-15.75(=formula 1.1437).

Table 1438. Compounds of formula I.A, wherein R² is R²-15.76(=formula 1.1438).

Table 1439. Compounds of formula I.A, wherein R² is R²-15.77(=formula 1.1439).

Table 1440. Compounds of formula I.A, wherein R² is R²-15.78(=formula 1.1440).

Table 1441. Compounds of formula I.A, wherein R² is R²-15.79(=formula 1.1441).

Table 1442. Compounds of formula I.A, wherein R² is R²-15.80(=formula 1.1442).

Table 1443. Compounds of formula I.A, wherein R² is R²-15.81(=formula 1.1443).

Table 1444. Compounds of formula I.A, wherein R² is R²-15.82(=formula 1.1444).

Table 1445. Compounds of formula I.A, wherein R² is R²-15.83(=formula 1.1445).

Table 1446. Compounds of formula I.A, wherein R² is R²-15.84(=formula 1.1446).

Table 1447. Compounds of formula I.A, wherein R² is R²-15.85(=formula 1.1447).

Table 1448. Compounds of formula I.A, wherein R² is R²-15.86(=formula 1.1448).

Table 1449. Compounds of formula I.A, wherein R² is R²-15.87(=formula 1.1449).

Table 1450. Compounds of formula I.A, wherein R² is R²-15.88(=formula 1.1450).

Table 1451. Compounds of formula I.A, wherein R² is R²-15.89(=formula 1.1451).

Table 1452. Compounds of formula I.A, wherein R² is R²-15.90(=formula 1.1452).

Table 1453. Compounds of formula I.A, wherein R² is R²-15.91(=formula 1.1453).

Table 1454. Compounds of formula I.A, wherein R² is R²-15.92(=formula 1.1454).

Table 1455. Compounds of formula I.A, wherein R² is R²-15.93(=formula 1.1455).

Table 1456. Compounds of formula I.A, wherein R² is R²-15.94(=formula 1.1456).

Table 1457. Compounds of formula I.A, wherein R² is R²-15.95(=formula 1.1457).

Table 1458. Compounds of formula I.A, wherein R² is R²-15.96(=formula 1.1458).

Table 1459. Compounds of formula I.A, wherein R² is R²-15.97(=formula 1.1459).

Table 1460. Compounds of formula I.A, wherein R² is R²-15.98(=formula 1.1460).

Table 1461. Compounds of formula I.A, wherein R² is R²-15.99(=formula 1.1461).

Table 1462. Compounds of formula I.A, wherein R² is R²-15.100(=formula 1.1462).

Table 1463. Compounds of formula I.A, wherein R² is R²-15.101(=formula 1.1463).

Table 1464. Compounds of formula I.A, wherein R² is R²-15.102(=formula 1.1464).

Table 1465. Compounds of formula I.A, wherein R² is R²-15.103(=formula 1.1465).

Table 1466. Compounds of formula I.A, wherein R² is R²-15.104(=formula 1.1466).

Table 1467. Compounds of formula I.A, wherein R² is R²-15.105(=formula 1.1467).

Table 1468. Compounds of formula I.A, wherein R² is R²-15.106(=formula 1.1468).

Table 1469. Compounds of formula I.A, wherein R² is R²-15.107(=formula 1.1469).

Table 1470. Compounds of formula I.A, wherein R² is R²-15.108(=formula 1.1470).

Table 1471. Compounds of formula I.A, wherein R² is R²-15.109(=formula 1.1471).

Table 1472. Compounds of formula I.A, wherein R² is R²-15.110(=formula 1.1472).

Table 1473. Compounds of formula I.A, wherein R² is R²-15.111(=formula 1.1473).

Table 1474. Compounds of formula I.A, wherein R² is R²-15.112(=formula 1.1475).

Table 1475. Compounds of formula I.A, wherein R² is R²-15.113(=formula 1.1475).

Table 1476. Compounds of formula I.A, wherein R² is R²-15.114(=formula 1.1476).

Table 1477. Compounds of formula I.A, wherein R² is R²-15.115(=formula 1.1477).

Table 1478. Compounds of formula I.A, wherein R² is R²-15.116(=formula 1.1478).

Table 1479. Compounds of formula I.A, wherein R² is R²-15.117(=formula 1.1479).

Table 1480. Compounds of formula I.A, wherein R² is R²-15.118(=formula 1.1480).

Table 1481. Compounds of formula I.A, wherein R² is R²-15.119(=formula 1.1481).

Table 1482. Compounds of formula I.A, wherein R² is R²-15.120(=formula 1.1482).

Table 1483. Compounds of formula I.A, wherein R² is R²-15.121(=formula 1.1483).

Table 1484. Compounds of formula I.A, wherein R² is R²-15.122(=formula 1.1484).

Table 1485. Compounds of formula I.A, wherein R² is R²-15.123(=formula 1.1485).

Table 1486. Compounds of formula I.A, wherein R² is R²-15.124(=formula 1.1486).

Table 1487. Compounds of formula I.A, wherein R² is R²-15.125(=formula 1.1487).

Table 1488. Compounds of formula I.A, wherein R² is R²-15.126(=formula 1.1488).

Table 1489. Compounds of formula I.A, wherein R² is R²-15.127(=formula 1.1489).

Table 1490. Compounds of formula I.A, wherein R² is R²-15.128(=formula 1.1490).

Table 1491. Compounds of formula I.A, wherein R² is R²-15.129(=formula 1.1491).

Table 1492. Compounds of formula I.A, wherein R² is R²-15.130(=formula 1.1492).

Table 1493. Compounds of formula I.A, wherein R² is R²-15.131(=formula 1.1493).

Table 1494. Compounds of formula I.A, wherein R² is R²-15.132(=formula 1.1494).

Table 1495. Compounds of formula I.A, wherein R² is R²-15.133(=formula 1.1495).

Table 1496. Compounds of formula I.A, wherein R² is R²-15.134(=formula 1.1496).

Table 1497. Compounds of formula I.A, wherein R² is R²-15.135(=formula 1.1497).

Table 1498. Compounds of formula I.A, wherein R² is R²-15.136(=formula 1.1498).

Table 1499. Compounds of formula I.A, wherein R² is R²-15.137(=formula 1.1499).

Table 1500. Compounds of formula I.A, wherein R² is R²-15.138(=formula 1.1500).

Table 1501. Compounds of formula I.A, wherein R² is R²-15.139(=formula 1.1501).

Table 1502. Compounds of formula I.A, wherein R² is R²-15.140(=formula 1.1502).

Table 1503. Compounds of formula I.A, wherein R² is R²-15.141(=formula 1.1503).

Table 1504. Compounds of formula I.A, wherein R² is R²-15.142(=formula 1.1504).

Table 1505. Compounds of formula I.A, wherein R² is R²-15.143(=formula 1.1505).

Table 1506. Compounds of formula I.A, wherein R² is R²-15.144(=formula 1.1506).

Table 1507. Compounds of formula I.A, wherein R² is R²-15.145(=formula 1.1507).

Table 1508. Compounds of formula I.A, wherein R² is R²-15.146(=formula 1.1508).

Table 1509. Compounds of formula I.A, wherein R² is R²-15.147(=formula 1.1509).

Table 1510. Compounds of formula I.A, wherein R² is R²-15.148(=formula 1.1510).

Table 1511. Compounds of formula I.A, wherein R² is R²-15.149(=formula 1.1511).

Table 1512. Compounds of formula I.A, wherein R² is R²-15.150(=formula 1.1512).

Table 1513. Compounds of formula I.A, wherein R² is R²-15.151(=formula 1.1513).

Table 1514. Compounds of formula I.A, wherein R² is R²-15.152(=formula 1.1514).

Table 1515. Compounds of formula I.A, wherein R² is R²-15.153(=formula 1.1515).

Table 1516. Compounds of formula I.A, wherein R² is R²-15.154(=formula 1.1516).

Table 1517. Compounds of formula I.A, wherein R² is R²-15.155(=formula 1.1517).

Table 1518. Compounds of formula I.A, wherein R² is R²-15.156(=formula 1.1518).

Table 1519. Compounds of formula I.A, wherein R² is R²-15.157(=formula 1.1519).

Table 1520. Compounds of formula I.A, wherein R² is R²-15.158(=formula 1.1520).

Table 1521. Compounds of formula I.A, wherein R² is R²-15.159(=formula 1.1521).

Table 1522. Compounds of formula I.A, wherein R² is R²-15.160(=formula 1.1522).

Table 1523. Compounds of formula I.A, wherein R² is R²-15.161(=formula 1.1523).

Table 1524. Compounds of formula I.A, wherein R² is R²-15.162(=formula 1.1524).

Table 1525. Compounds of formula I.A, wherein R² is R²-15.163(=formula 1.1525).

Table 1526. Compounds of formula I.A, wherein R² is R²-15.164(=formula 1.1526).

Table 1527. Compounds of formula I.A, wherein R² is R²-15.165(=formula 1.1527).

Table 1528. Compounds of formula I.A, wherein R² is R²-15.166(=formula 1.1528).

Table 1529. Compounds of formula I.A, wherein R² is R²-15.167(=formula 1.1529).

Table 1530. Compounds of formula I.A, wherein R² is R²-15.168(=formula 1.1530).

Table 1531. Compounds of formula I.A, wherein R² is R²-15.169(=formula 1.1531).

Table 1532. Compounds of formula I.A, wherein R² is R²-15.170(=formula 1.1532).

Table 1533. Compounds of formula I.A, wherein R² is R²-15.171(=formula 1.1533).

Table 1534. Compounds of formula I.A, wherein R² is R²-15.172(=formula 1.1534).

Table 1535. Compounds of formula I.A, wherein R² is R²-15.173(=formula 1.1535).

Table 1536. Compounds of formula I.A, wherein R² is R²-15.174(=formula 1.1536).

Table 1537. Compounds of formula I.A, wherein R² is R²-15.175(=formula 1.1537).

Table 1538. Compounds of formula I.A, wherein R² is R²-15.176(=formula 1.1538).

Table 1539. Compounds of formula I.A, wherein R² is R²-15.177(=formula 1.1539).

Table 1540. Compounds of formula I.A, wherein R² is R²-15.178(=formula 1.1540).

Table 1541. Compounds of formula I.A, wherein R² is R²-15.179(=formula 1.1541).

Table 1542. Compounds of formula I.A, wherein R² is R²-15.180(=formula 1.1542).

Table 1543. Compounds of formula I.A, wherein R² is R²-15.181(=formula 1.1543).

Table 1544. Compounds of formula I.A, wherein R² is R²-15.182(=formula 1.1544).

Table 1545. Compounds of formula I.A, wherein R² is R²-15.183(=formula 1.1545).

Table 1546. Compounds of formula I.A, wherein R² is R²-15.184(=formula 1.1546).

Table 1547. Compounds of formula I.A, wherein R² is R²-15.185(=formula 1.1547).

Table 1548. Compounds of formula I.A, wherein R² is R²-15.186(=formula 1.1548).

Table 1549. Compounds of formula I.A, wherein R² is R²-15.187(=formula 1.1549).

Table 1550. Compounds of formula I.A, wherein R² is R²-15.188(=formula 1.1550).

Table 1551. Compounds of formula I.A, wherein R² is R²-15.189(=formula 1.1551).

Table 1552. Compounds of formula I.A, wherein R² is R²-15.190(=formula 1.1552).

Table 1553. Compounds of formula I.A, wherein R² is R²-15.191(=formula 1.1553).

Table 1554. Compounds of formula I.A, wherein R² is R²-15.192(=formula 1.1554).

Table 1555. Compounds of formula I.A, wherein R² is R²-15.193(=formula 1.1555).

Table 1556. Compounds of formula I.A, wherein R² is R²-15.194(=formula 1.1556).

Table 1557. Compounds of formula I.A, wherein R² is R²-15.195(=formula 1.1557).

Table 1558. Compounds of formula I.A, wherein R² is R²-15.196(=formula 1.1558).

Table 1559. Compounds of formula I.A, wherein R² is R²-15.197(=formula 1.1559).

Table 1560. Compounds of formula I.A, wherein R² is R²-15.198(=formula 1.1560).

Table 1561. Compounds of formula I.A, wherein R² is R²-15.199(=formula 1.1561).

Table 1562. Compounds of formula I.A, wherein R² is R²-15.200(=formula 1.1562).

Table 1563. Compounds of formula I.A, wherein R² is R²-15.201(=formula 1.1563).

Table 1564. Compounds of formula I.A, wherein R² is R²-15.202(=formula 1.1564).

Table 1565. Compounds of formula I.A, wherein R² is R²-15.203(=formula 1.1565).

Table 1566. Compounds of formula I.A, wherein R² is R²-15.204(=formula 1.1566).

Table 1567. Compounds of formula I.A, wherein R² is R²-15.205(=formula 1.1567).

Table 1568. Compounds of formula I.A, wherein R² is R²-15.206(=formula 1.1568).

Table 1569. Compounds of formula I.A, wherein R² is R²-15.207(=formula 1.1569).

Table 1570. Compounds of formula I.A, wherein R² is R²-15.208(=formula 1.1570).

Table 1571. Compounds of formula I.A, wherein R² is R²-15.209(=formula 1.1571).

Table 1572. Compounds of formula I.A, wherein R² is R²-15.210(=formula 1.1572).

Table 1573. Compounds of formula I.A, wherein R² is R²-15.211(=formula 1.1573).

Table 1574. Compounds of formula I.A, wherein R² is R²-15.212(=formula 1.1574).

Table 1575. Compounds of formula I.A, wherein R² is R²-15.213(=formula 1.1575).

Table 1576. Compounds of formula I.A, wherein R² is R²-15.214(=formula 1.1576).

Table 1577. Compounds of formula I.A, wherein R² is R²-15.215(=formula 1.1577).

Table 1578. Compounds of formula I.A, wherein R² is R²-15.216(=formula 1.1578).

Table 1579. Compounds of formula I.A, wherein R² is R²-15.217(=formula 1.1579).

Table 1580. Compounds of formula I.A, wherein R² is R²-15.218(=formula 1.1580).

Table 1581. Compounds of formula I.A, wherein R² is R²-15.219(=formula 1.1581).

Table 1582. Compounds of formula I.A, wherein R² is R²-15.220(=formula 1.1582).

Table 1583. Compounds of formula I.A, wherein R² is R²-15.221(=formula 1.1583).

Table 1584. Compounds of formula I.A, wherein R² is R²-15.222(=formula 1.1584).

Table 1585. Compounds of formula I.A, wherein R² is R²-15.223(=formula 1.1585).

Table 1586. Compounds of formula I.A, wherein R² is R²-15.224(=formula 1.1586).

Table 1587. Compounds of formula I.A, wherein R² is R²-15.225(=formula 1.1587).

Table 1588. Compounds of formula I.A, wherein R² is R²-15.226(=formula 1.1588).

Table 1589. Compounds of formula I.A, wherein R² is R²-15.227(=formula 1.1589).

Table 1590. Compounds of formula I.A, wherein R² is R²-15.228(=formula 1.1590).

Table 1591. Compounds of formula I.A, wherein R² is R²-15.229(=formula 1.1591).

Table 1592. Compounds of formula I.A, wherein R² is R²-15.230(=formula 1.1592).

Table 1593. Compounds of formula I.A, wherein R² is R²-15.231(=formula 1.1593).

Table 1594. Compounds of formula I.A, wherein R² is R²-15.232(=formula 1.1594).

Table 1595. Compounds of formula I.A, wherein R² is R²-15.233(=formula 1.1595).

Table 1596. Compounds of formula I.A, wherein R² is R²-15.234(=formula 1.1596).

Table 1597. Compounds of formula I.A, wherein R² is R²-15.235(=formula 1.1597).

Table 1598. Compounds of formula I.A, wherein R² is R²-15.236(=formula 1.1598).

Table 1599. Compounds of formula I.A, wherein R² is R²-15.237(=formula 1.1599).

Table 1600. Compounds of formula I.A, wherein R² is R²-15.238(=formula 1.1600).

Table 1601. Compounds of formula I.A, wherein R² is R²-15.239(=formula 1.1601).

Table 1602. Compounds of formula I.A, wherein R² is R²-15.240(=formula 1.1602).

Table 1603. Compounds of formula I.A, wherein R² is R²-15.241(=formula 1.1603).

Table 1604. Compounds of formula I.A, wherein R² is R²-15.242(=formula 1.1604).

Table 1605. Compounds of formula I.A, wherein R² is R²-15.243(=formula 1.1605).

Table 1606. Compounds of formula I.A, wherein R² is R²-15.244(=formula 1.1606).

Table 1607. Compounds of formula I.A, wherein R² is R²-15.245(=formula 1.1607).

Table 1608. Compounds of formula I.A, wherein R² is R²-15.246(=formula 1.1608).

Table 1609. Compounds of formula I.A, wherein R² is R²-15.247(=formula 1.1609).

Table 1610. Compounds of formula I.A, wherein R² is R²-15.248(=formula 1.1610).

Table 1611. Compounds of formula I.A, wherein R² is R²-15.249(=formula 1.1611).

Table 1612. Compounds of formula I.A, wherein R² is R²-15.250(=formula 1.1612).

Table 1613. Compounds of formula I.A, wherein R² is R²-15.251(=formula 1.1613).

Table 1614. Compounds of formula I.A, wherein R² is R²-15.252(=formula 1.1614).

Table 1615. Compounds of formula I.A, wherein R² is R²-15.253(=formula 1.1615).

Table 1616. Compounds of formula I.A, wherein R² is R²-15.254(=formula 1.1616).

Table 1617. Compounds of formula I.A, wherein R² is R²-15.255(=formula 1.1617).

Table 1618. Compounds of formula I.A, wherein R² is R²-15.256(=formula 1.1618).

Table 1619. Compounds of formula I.A, wherein R² is R²-15.257(=formula 1.1619).

Table 1620. Compounds of formula I.A, wherein R² is R²-15.258(=formula 1.1620).

Table 1621. Compounds of formula I.A, wherein R² is R²-15.259(=formula 1.1621).

Table 1622. Compounds of formula I.A, wherein R² is R²-15.260(=formula 1.1622).

Table 1623. Compounds of formula I.A, wherein R² is R²-15.261(=formula 1.1623).

Table 1624. Compounds of formula I.A, wherein R² is R²-15.262(=formula 1.1624).

Table 1625. Compounds of formula I.A, wherein R² is R²-15.263(=formula 1.1625).

Table 1626. Compounds of formula I.A, wherein R² is R²-15.264(=formula 1.1626).

Table 1627. Compounds of formula I.A, wherein R² is R²-15.265(=formula 1.1627).

Table 1628. Compounds of formula I.A, wherein R² is R²-15.266(=formula 1.1628).

Table 1629. Compounds of formula I.A, wherein R² is R²-15.267(=formula 1.1629).

Table 1630. Compounds of formula I.A, wherein R² is R²-15.268(=formula 1.1630).

Table 1631. Compounds of formula I.A, wherein R² is R²-15.269(=formula 1.1631).

Table 1632. Compounds of formula I.A, wherein R² is R²-15.270(=formula 1.1632).

Table 1633. Compounds of formula I.A, wherein R² is R²-15.271(=formula 1.1633).

Table 1634. Compounds of formula I.A, wherein R² is R²-15.272(=formula 1.1634).

Table 1635. Compounds of formula I.A, wherein R² is R²-15.273(=formula 1.1635).

Table 1636. Compounds of formula I.A, wherein R² is R²-15.274(=formula 1.1636).

Table 1637. Compounds of formula I.A, wherein R² is R²-15.275(=formula 1.1637).

Table 1638. Compounds of formula I.A, wherein R² is R²-15.276(=formula 1.1638).

Table 1639. Compounds of formula I.A, wherein R² is R²-15.277(=formula 1.1639).

Table 1640. Compounds of formula I.A, wherein R² is R²-15.278(=formula 1.1640).

Table 1641. Compounds of formula I.A, wherein R² is R²-15.279(=formula 1.1641).

Table 1642. Compounds of formula I.A, wherein R² is R²-15.280(=formula 1.1642).

Table 1643. Compounds of formula I.A, wherein R² is R²-15.281(=formula 1.1643).

Table 1644. Compounds of formula I.A, wherein R² is R²-15.282(=formula 1.1644).

Table 1645. Compounds of formula I.A, wherein R² is R²-15.283(=formula 1.1645).

Table 1646. Compounds of formula I.A, wherein R² is R²-15.284(=formula 1.1646).

Table 1647. Compounds of formula I.A, wherein R² is R²-15.285(=formula 1.1647).

Table 1648. Compounds of formula I.A, wherein R² is R²-15.286(=formula 1.1648).

Table 1649. Compounds of formula I.A, wherein R² is R²-15.287(=formula 1.1649).

Table 1650. Compounds of formula I.A, wherein R² is R²-15.288(=formula 1.1650).

Table 1651. Compounds of formula I.A, wherein R² is R²-15.289(=formula 1.1651).

Table 1652. Compounds of formula I.A, wherein R² is R²-15.290(=formula 1.1652).

Table 1653. Compounds of formula I.A, wherein R² is R²-15.291(=formula 1.1653).

Table 1654. Compounds of formula I.A, wherein R² is R²-15.292(=formula 1.1654).

Table 1655. Compounds of formula I.A, wherein R² is R²-15.293(=formula 1.1655).

Table 1656. Compounds of formula I.A, wherein R² is R²-15.294(=formula 1.1656).

Table 1657. Compounds of formula I.A, wherein R² is R²-15.295(=formula 1.1657).

Table 1658. Compounds of formula I.A, wherein R² is R²-15.296(=formula 1.1658).

Table 1659. Compounds of formula I.A, wherein R² is R²-15.297(=formula 1.1659).

Table 1660. Compounds of formula I.A, wherein R² is R²-15.298(=formula 1.1660).

Table 1661. Compounds of formula I.A, wherein R² is R²-15.299(=formula 1.1661).

Table 1662. Compounds of formula I.A, wherein R² is R²-15.300(=formula 1.1662).

Table 1663. Compounds of formula I.A, wherein R² is R²-15.301(=formula 1.1663).

Table 1664. Compounds of formula I.A, wherein R² is R²-15.302(=formula 1.1664).

Table 1665. Compounds of formula I.A, wherein R² is R²-15.303(=formula 1.1665).

Table 1666. Compounds of formula I.A, wherein R² is R²-15.304(=formula 1.1666).

Table 1667. Compounds of formula I.A, wherein R² is R²-15.305(=formula 1.1667).

Table 1668. Compounds of formula I.A, wherein R² is R²-15.306(=formula 1.1668).

Table 1669. Compounds of formula I.A, wherein R² is R²-15.307(=formula 1.1669).

Table 1670. Compounds of formula I.A, wherein R² is R²-15.308(=formula 1.1670).

Table 1671. Compounds of formula I.A, wherein R² is R²-15.309(=formula 1.1671).

Table 1672. Compounds of formula I.A, wherein R² is R²-15.310(=formula 1.1672).

Table 1673. Compounds of formula I.A, wherein R² is R²-15.311(=formula 1.1673).

Table 1674. Compounds of formula I.A, wherein R² is R²-15.312(=formula 1.1674).

Table 1675. Compounds of formula I.A, wherein R² is R²-15.313(=formula 1.1675).

Table 1676. Compounds of formula I.A, wherein R² is R²-15.314(=formula 1.1676).

Table 1677. Compounds of formula I.A, wherein R² is R²-15.315(=formula 1.1676).

Table 1678. Compounds of formula I.A, wherein R² is R²-15.316(=formula 1.1678).

Table 1679. Compounds of formula I.A, wherein R² is R²-15.317(=formula 1.1679).

Table 1680. Compounds of formula I.A, wherein R² is R²-15.318(=formula 1.1680).

Table 1681. Compounds of formula I.A, wherein R² is R²-15.319(=formula 1.1681).

Table 1682. Compounds of formula I.A, wherein R² is R²-15.320(=formula 1.1682).

Table 1683. Compounds of formula I.A, wherein R² is R²-15.321(=formula 1.1683).

Table 1684. Compounds of formula I.A, wherein R² is R²-15.322(=formula 1.1684).

Table 1685. Compounds of formula I.A, wherein R² is R²-15.323(=formula 1.1685).

Table 1686. Compounds of formula I.A, wherein R² is R²-15.324(=formula 1.1686).

Table 1687. Compounds of formula I.A, wherein R² is R²-15.325(=formula 1.1687).

Table 1688. Compounds of formula I.A, wherein R² is R²-15.326(=formula 1.1688).

Table 1689. Compounds of formula I.A, wherein R² is R²-15.327(=formula 1.1689).

Table 1690. Compounds of formula I.A, wherein R² is R²-15.328(=formula 1.1690).

Table 1691. Compounds of formula I.A, wherein R² is R²-15.329(=formula 1.1691).

Table 1692. Compounds of formula I.A, wherein R² is R²-15.330(=formula 1.1692).

Table 1693. Compounds of formula I.A, wherein R² is R²-15.331(=formula 1.1693).

Table 1694. Compounds of formula I.A, wherein R² is R²-15.332(=formula 1.1694).

Table 1695. Compounds of formula I.A, wherein R² is R²-15.333(=formula 1.1695).

Table 1696. Compounds of formula I.A, wherein R² is R²-15.334(=formula 1.1696).

Table 1697. Compounds of formula I.A, wherein R² is R²-15.335(=formula 1.1697).

Table 1698. Compounds of formula I.A, wherein R² is R²-15.336(=formula 1.1698).

Table 1699. Compounds of formula I.A, wherein R² is R²-15.334(=formula 1.1699).

Table 1700. Compounds of formula I.A, wherein R² is R²-15.338(=formula 1.1700).

Table 1701. Compounds of formula I.A, wherein R² is R²-15.339(=formula 1.1701).

Table 1702. Compounds of formula I.A, wherein R² is R²-15.340(=formula 1.1702).

Table 1703. Compounds of formula I.A, wherein R² is R²-15.341(=formula 1.1703).

Table 1704. Compounds of formula I.A, wherein R² is R²-15.342(=formula 1.1704).

Table 1705. Compounds of formula I.A, wherein R² is R²-15.343(=formula 1.1705).

Table 1706. Compounds of formula I.A, wherein R² is R²-15.344(=formula 1.1706).

Table 1707. Compounds of formula I.A, wherein R² is R²-15.345(=formula 1.1707).

Table 1708. Compounds of formula I.A, wherein R² is R²-15.346(=formula 1.1708).

Table 1709. Compounds of formula I.A, wherein R² is R²-15.347(=formula 1.1709).

Table 1710. Compounds of formula I.A, wherein R² is R²-15.348(=formula 1.1710).

Table 1711. Compounds of formula I.A, wherein R² is R²-15.349(=formula 1.1711).

Table 1712. Compounds of formula I.A, wherein R² is R²-15.350(=formula 1.1712).

Table 1713. Compounds of formula I.A, wherein R² is R²-15.351(=formula 1.1713).

Table 1714. Compounds of formula I.A, wherein R² is R²-15.352(=formula 1.1714).

Table 1715. Compounds of formula I.A, wherein R² is R²-15.353(=formula 1.1715).

Table 1716. Compounds of formula I.A, wherein R² is R²-15.354(=formula 1.1716).

Table 1717. Compounds of formula I.A, wherein R² is R²-15.355(=formula 1.1717).

Table 1718. Compounds of formula I.A, wherein R² is R²-15.356(=formula 1.1718).

Table 1719. Compounds of formula I.A, wherein R² is R²-15.357(=formula 1.1719).

Table 1720. Compounds of formula I.A, wherein R² is R²-15.358(=formula 1.1720).

Table 1721. Compounds of formula I.A, wherein R² is R²-15.359(=formula 1.1721).

Table 1722. Compounds of formula I.A, wherein R² is R²-15.360(=formula 1.1722).

Table 1723. Compounds of formula I.A, wherein R² is R²-15.361(=formula 1.1723).

Table 1724. Compounds of formula I.A, wherein R² is R²-15.362(=formula 1.1724).

Table 1725. Compounds of formula I.A, wherein R² is R²-15.363(=formula 1.1725).

Table 1726. Compounds of formula I.A, wherein R² is R²-15.364(=formula 1.1726).

Table 1727. Compounds of formula I.A, wherein R² is R²-15.365(=formula 1.1727).

Table 1728. Compounds of formula I.A, wherein R² is R²-15.366(=formula 1.1728).

Table 1729. Compounds of formula I.A, wherein R² is R²-15.367(=formula 1.1729).

Table 1730. Compounds of formula I.A, wherein R² is R²-15.368(=formula 1.1730).

Table 1731. Compounds of formula I.A, wherein R² is R²-15.369(=formula 1.1731).

Table 1732. Compounds of formula I.A, wherein R² is R²-15.370(=formula 1.1732).

Table 1733. Compounds of formula I.A, wherein R² is R²-15.371(=formula 1.1733).

Table 1734. Compounds of formula I.A, wherein R² is R²-15.372(=formula 1.1734).

Table 1735. Compounds of formula I.A, wherein R² is R²-15.373(=formula 1.1735).

Table 1736. Compounds of formula I.A, wherein R² is R²-15.374(=formula 1.1736).

Table 1737. Compounds of formula I.A, wherein R² is R²-15.375(=formula 1.1737).

Table 1738. Compounds of formula I.A, wherein R² is R²-15.376(=formula 1.1738).

Table 1739. Compounds of formula I.A, wherein R² is R²-15.377(=formula 1.1739).

Table 1740. Compounds of formula I.A, wherein R² is R²-15.378(=formula 1.1740).

Table 1741. Compounds of formula I.A, wherein R² is R²-15.379(=formula 1.1741).

Table 1742. Compounds of formula I.A, wherein R² is R²-15.380(=formula 1.1742).

Table 1743. Compounds of formula I.A, wherein R² is R²-15.381(=formula 1.1743).

Table 1744. Compounds of formula I.A, wherein R² is R²-15.382(=formula 1.1744).

Table 1745. Compounds of formula I.A, wherein R² is R²-15.383(=formula 1.1745).

Table 1746. Compounds of formula I.A, wherein R² is R²-15.384(=formula 1.1746).

Table 1747. Compounds of formula I.A, wherein R² is R²-15.385(=formula 1.1747).

Table 1748. Compounds of formula I.A, wherein R² is R²-15.386(=formula 1.1748).

Table 1749. Compounds of formula I.A, wherein R² is R²-15.387(=formula 1.1749).

Table 1750. Compounds of formula I.A, wherein R² is R²-15.388(=formula 1.1750).

Table 1751. Compounds of formula I.A, wherein R² is R²-15.389(=formula 1.1751).

Table 1752. Compounds of formula I.A, wherein R² is R²-15.390(=formula 1.1752).

Table 1753. Compounds of formula I.A, wherein R² is R²-15.391(=formula 1.1753).

Table 1754. Compounds of formula I.A, wherein R² is R²-15.392(=formula 1.1754).

Table 1755. Compounds of formula I.A, wherein R² is R²-15.393(=formula 1.1755).

Table 1756. Compounds of formula I.A, wherein R² is R²-15.394(=formula 1.1756).

Table 1757. Compounds of formula I.A, wherein R² is R²-15.395(=formula 1.1757).

Table 1758. Compounds of formula I.A, wherein R² is R²-15.396(=formula 1.1758).

Table 1759. Compounds of formula I.A, wherein R² is R²-15.397(=formula 1.1759).

Table 1760. Compounds of formula I.A, wherein R² is R²-15.398(=formula 1.1760).

Table 1761. Compounds of formula I.A, wherein R² is R²-15.399(=formula 1.1761).

Table 1762. Compounds of formula I.A, wherein R² is R²-15.400(=formula 1.1762).

Table 1763. Compounds of formula I.A, wherein R² is R²-15.401(=formula 1.1763).

Table 1764. Compounds of formula I.A, wherein R² is R²-15.402(=formula 1.1764).

Table 1765. Compounds of formula I.A, wherein R² is R²-15.403(=formula 1.1765).

Table 1766. Compounds of formula I.A, wherein R² is R²-15.404(=formula 1.1766).

Table 1767. Compounds of formula I.A, wherein R² is R²-15.405(=formula 1.1767).

Table 1768. Compounds of formula I.A, wherein R² is R²-15.406(=formula 1.1768).

Table 1769. Compounds of formula I.A, wherein R² is R²-15.407(=formula 1.1769).

Table 1770. Compounds of formula I.A, wherein R² is R²-15.408(=formula 1.1770).

Table 1771. Compounds of formula I.A, wherein R² is R²-15.409(=formula 1.1771).

Table 1772. Compounds of formula I.A, wherein R² is R²-15.410(=formula 1.1772).

Table 1773. Compounds of formula I.A, wherein R² is R²-15.411(=formula 1.1773).

Table 1774. Compounds of formula I.A, wherein R² is R²-15.412(=formula 1.1774).

Table 1775. Compounds of formula I.A, wherein R² is R²-15.413(=formula 1.1775).

Table 1776. Compounds of formula I.A, wherein R² is R²-15.414(=formula 1.1776).

Table 1777. Compounds of formula I.A, wherein R² is R²-15.415(=formula 1.1777).

Table 1778. Compounds of formula I.A, wherein R² is R²-15.416(=formula 1.1778).

Table 1779. Compounds of formula I.A, wherein R² is R²-15.417(=formula 1.1779).

Table 1780. Compounds of formula I.A, wherein R² is R²-15.418(=formula 1.1780).

Table 1781. Compounds of formula I.A, wherein R² is R²-15.419(=formula 1.1781).

Table 1782. Compounds of formula I.A, wherein R² is R²-15.420(=formula 1.1782).

Table 1783. Compounds of formula I.A, wherein R² is R²-15.421(=formula 1.1783).

Table 1784. Compounds of formula I.A, wherein R² is R²-15.422(=formula 1.1784).

Table 1785. Compounds of formula I.A, wherein R² is R²-15.423(=formula 1.1785).

Table 1786. Compounds of formula I.A, wherein R² is R²-15.424(=formula 1.1786).

Table 1787. Compounds of formula I.A, wherein R² is R²-15.425(=formula 1.1787).

Table 1788. Compounds of formula I.A, wherein R² is R²-15.426(=formula 1.1788).

Table 1789. Compounds of formula I.A, wherein R² is R²-15.427(=formula 1.1789).

Table 1790. Compounds of formula I.A, wherein R² is R²-15.428(=formula 1.1790).

Table 1791. Compounds of formula I.A, wherein R² is R²-15.429(=formula 1.1791).

Table 1792. Compounds of formula I.A, wherein R² is R²-15.430(=formula 1.1792).

Table 1793. Compounds of formula I.A, wherein R² is R²-15.431(=formula 1.1793).

Table 1794. Compounds of formula I.A, wherein R² is R²-15.432(=formula 1.1794).

Table 1795. Compounds of formula I.A, wherein R² is R²-15.433(=formula 1.1795).

Table 1796. Compounds of formula I.A, wherein R² is R²-15.434(=formula 1.1796).

Table 1797. Compounds of formula I.A, wherein R² is R²-15.435(=formula 1.1797).

Table 1798. Compounds of formula I.A, wherein R² is R²-15.436(=formula 1.1798).

Table 1799. Compounds of formula I.A, wherein R² is R²-15.437(=formula 1.1799).

Table 1800. Compounds of formula I.A, wherein R² is R²-15.438(=formula 1.1800).

Table 1801. Compounds of formula I.A, wherein R² is R²-15.439(=formula 1.1801).

Table 1802. Compounds of formula I.A, wherein R² is R²-15.440(=formula 1.1802).

Table 1803. Compounds of formula I.A, wherein R² is R²-15.441(=formula 1.1803).

Table 1804. Compounds of formula I.A, wherein R² is R²-15.442(=formula 1.1804).

Table 1805. Compounds of formula I.A, wherein R² is R²-15.443(=formula 1.1805).

Table 1806. Compounds of formula I.A, wherein R² is R²-15.444(=formula 1.1806).

Table 1807. Compounds of formula I.A, wherein R² is R²-15.445(=formula 1.1807).

Table 1808. Compounds of formula I.A, wherein R² is R²-15.446(=formula 1.1808).

Table 1809. Compounds of formula I.A, wherein R² is R²-15.447(=formula 1.1809).

Table 1810. Compounds of formula I.A, wherein R² is R²-15.448(=formula 1.1810).

Table 1811. Compounds of formula I.A, wherein R² is R²-15.449(=formula 1.1811).

Table 1812. Compounds of formula I.A, wherein R² is R²-15.450(=formula 1.1812).

Table 1813. Compounds of formula I.A, wherein R² is R²-15.451(=formula 1.1813).

Table 1814. Compounds of formula I.A, wherein R² is R²-15.452(=formula 1.1814).

Table 1815. Compounds of formula I.A, wherein R² is R²-15.453(=formula 1.1815).

Table 1816. Compounds of formula I.A, wherein R² is R²-15.454(=formula 1.1816).

Table 1817. Compounds of formula I.A, wherein R² is R²-15.455(=formula 1.1817).

Table 1818. Compounds of formula I.A, wherein R² is R²-15.456(=formula 1.1818).

Table 1819. Compounds of formula I.A, wherein R² is R²-15.457(=formula 1.1819).

Table 1820. Compounds of formula I.A, wherein R² is R²-15.458(=formula 1.1820).

Table 1821. Compounds of formula I.A, wherein R² is R²-15.459(=formula 1.1821).

Table 1822. Compounds of formula I.A, wherein R² is R²-15.460(=formula 1.1822).

Table 1823. Compounds of formula I.A, wherein R² is R²-15.461(=formula 1.1823).

Table 1824. Compounds of formula I.A, wherein R² is R²-15.462(=formula 1.1824).

Table 1825. Compounds of formula I.A, wherein R² is R²-15.463(=formula 1.1825).

Table 1826. Compounds of formula I.A, wherein R² is R²-15.464(=formula 1.1826).

Table 1827. Compounds of formula I.A, wherein R² is R²-15.465(=formula 1.1827).

Table 1828. Compounds of formula I.A, wherein R² is R²-15.466(=formula 1.1828).

Table 1829. Compounds of formula I.A, wherein R² is R²-15.467(=formula 1.1829).

Table 1830. Compounds of formula I.A, wherein R² is R²-15.468(=formula 1.1830).

Table 1831. Compounds of formula I.A, wherein R² is R²-15.469(=formula 1.1831).

Table 1832. Compounds of formula I.A, wherein R² is R²-15.470(=formula 1.1832).

Table 1833. Compounds of formula I.A, wherein R² is R²-15.471(=formula 1.1833).

Table 1834. Compounds of formula I.A, wherein R² is R²-15.472(=formula 1.1834).

Table 1835. Compounds of formula I.A, wherein R² is R²-15.473(=formula 1.1835).

Table 1836. Compounds of formula I.A, wherein R² is R²-15.474(=formula 1.1836).

Table 1837. Compounds of formula I.A, wherein R² is R²-15.475(=formula 1.1837).

Table 1838. Compounds of formula I.A, wherein R² is R²-15.476(=formula 1.1838).

Table 1839. Compounds of formula I.A, wherein R² is R²-15.477(=formula 1.1839).

Table 1840. Compounds of formula I.A, wherein R² is R²-15.478(=formula 1.1840).

Table 1841. Compounds of formula I.A, wherein R² is R²-15.479(=formula 1.1841).

Table 1842. Compounds of formula I.A, wherein R² is R²-15.480(=formula 1.1842).

Table 1843. Compounds of formula I.A, wherein R² is R²-15.481(=formula 1.1843).

Table 1844. Compounds of formula I.A, wherein R² is R²-15.482(=formula 1.1844).

Table 1845. Compounds of formula I.A, wherein R² is R²-15.483(=formula 1.1845).

Table 1846. Compounds of formula I.A, wherein R² is R²-15.484(=formula 1.1846).

Table 1847. Compounds of formula I.A, wherein R² is R²-15.485(=formula 1.1847).

Table 1848. Compounds of formula I.A, wherein R² is R²-15.486(=formula 1.1848).

Table 1849. Compounds of formula I.A, wherein R² is R²-15.487(=formula 1.1849).

Table 1850. Compounds of formula I.A, wherein R² is R²-15.488(=formula 1.1850).

Table 1851. Compounds of formula I.A, wherein R² is R²-15.489(=formula 1.1851).

Table 1852. Compounds of formula I.A, wherein R² is R²-15.490(=formula 1.1852).

Table 1853. Compounds of formula I.A, wherein R² is R²-15.491(=formula 1.1853).

Table 1854. Compounds of formula I.A, wherein R² is R²-15.492(=formula 1.1854).

Table 1855. Compounds of formula I.A, wherein R² is R²-15.493(=formula 1.1855).

Table 1856. Compounds of formula I.A, wherein R² is R²-15.494(=formula 1.1856).

Table 1857. Compounds of formula I.A, wherein R² is R²-15.495(=formula 1.1857).

Table 1858. Compounds of formula I.A, wherein R² is R²-15.496(=formula 1.1858).

Table 1859. Compounds of formula I.A, wherein R² is R²-15.497(=formula 1.1859).

Table 1860. Compounds of formula I.A, wherein R² is R²-15.498(=formula 1.1860).

Table 1861. Compounds of formula I.A, wherein R² is R²-15.499(=formula 1.1861).

Table 1862. Compounds of formula I.A, wherein R² is R²-15.500(=formula 1.1862).

Table 1863. Compounds of formula I.A, wherein R² is R²-15.501(=formula 1.1863).

Table 1864. Compounds of formula I.A, wherein R² is R²-15.502(=formula 1.1864).

Table 1865. Compounds of formula I.A, wherein R² is R²-15.503(=formula 1.1865).

Table 1866. Compounds of formula I.A, wherein R² is R²-15.504(=formula 1.1866).

Table 1867. Compounds of formula I.A, wherein R² is R²-15.505(=formula 1.1867).

Table 1868. Compounds of formula I.A, wherein R² is R²-15.506(=formula 1.1868).

Table 1869. Compounds of formula I.A, wherein R² is R²-15.507(=formula 1.1869).

Table 1870. Compounds of formula I.A, wherein R² is R²-15.508(=formula 1.1870).

Table 1871. Compounds of formula I.A, wherein R² is R²-15.509(=formula 1.1871).

Table 1872. Compounds of formula I.A, wherein R² is R²-15.510(=formula 1.1872).

Table 1873. Compounds of formula I.A, wherein R² is R²-15.511(=formula 1.1873).

Table 1874. Compounds of formula I.A, wherein R² is R²-15.512(=formula 1.1874).

Table 1875. Compounds of formula I.A, wherein R² is R²-15.513(=formula 1.1875).

Table 1876. Compounds of formula I.A, wherein R² is R²-15.514(=formula 1.1876).

Table 1877. Compounds of formula I.A, wherein R² is R²-15.515(=formula 1.1877).

Table 1878. Compounds of formula I.A, wherein R² is R²-15.516(=formula 1.1878).

Table 1879. Compounds of formula I.A, wherein R² is R²-15.517(=formula 1.1879).

Table 1880. Compounds of formula I.A, wherein R² is R²-15.518(=formula 1.1880).

Table 1881. Compounds of formula I.A, wherein R² is R²-15.519(=formula 1.1881).

Table 1882. Compounds of formula I.A, wherein R² is R²-15.520(=formula 1.1882).

Table 1883. Compounds of formula I.A, wherein R² is R²-15.521(=formula 1.1883).

Table 1884. Compounds of formula I.A, wherein R² is R²-15.522(=formula 1.1884).

Table 1885. Compounds of formula I.A, wherein R² is R²-15.523(=formula 1.1885).

Table 1886. Compounds of formula I.A, wherein R² is R²-15.524(=formula 1.1886).

Table 1887. Compounds of formula I.A, wherein R² is R²-15.525(=formula 1.1887).

Table 1888. Compounds of formula I.A, wherein R² is R²-15.526(=formula 1.1888).

Table 1889. Compounds of formula I.A, wherein R² is R²-15.527(=formula 1.1889).

Table 1890. Compounds of formula I.A, wherein R² is R²-15.528(=formula 1.1890).

Table 1891. Compounds of formula I.A, wherein R² is R²-15.529(=formula 1.1891).

Table 1892. Compounds of formula I.A, wherein R² is R²-15.530(=formula 1.1892).

Table 1893. Compounds of formula I.A, wherein R² is R²-15.531(=formula 1.1893).

Table 1894. Compounds of formula I.A, wherein R² is R²-15.532(=formula 1.1894).

Table 1895. Compounds of formula I.A, wherein R² is R²-15.533(=formula 1.1895).

Table 1896. Compounds of formula I.A, wherein R² is R²-15.534(=formula 1.1896).

Table 1897. Compounds of formula I.A, wherein R² is R²-15.535(=formula 1.1897).

Table 1898. Compounds of formula I.A, wherein R² is R²-15.536(=formula 1.1898).

Table 1899. Compounds of formula I.A, wherein R² is R²-15.537(=formula 1.1899).

Table 1900. Compounds of formula I.A, wherein R² is R²-15.538(=formula 1.1900).

Table 1901. Compounds of formula I.A, wherein R² is R²-15.539(=formula 1.1901).

Table 1902. Compounds of formula I.A, wherein R² is R²-15.540(=formula 1.1902).

Table 1903. Compounds of formula I.A, wherein R² is R²-15.541(=formula 1.1903).

Table 1904. Compounds of formula I.A, wherein R² is R²-15.542(=formula 1.1904).

Table 1905. Compounds of formula I.A, wherein R² is R²-15.543(=formula 1.1905).

Table 1906. Compounds of formula I.A, wherein R² is R²-15.544(=formula 1.1906).

Table 1907. Compounds of formula I.A, wherein R² is R²-15.545(=formula 1.1907).

Table 1908. Compounds of formula I.A, wherein R² is R²-15.546(=formula 1.1908).

Table 1909. Compounds of formula I.A, wherein R² is R²-15.547(=formula 1.1909).

Table 1910. Compounds of formula I.A, wherein R² is R²-15.548(=formula 1.1910).

Table 1911. Compounds of formula I.A, wherein R² is R²-15.549(=formula 1.1911).

Table 1912. Compounds of formula I.A, wherein R² is R²-15.550(=formula 1.1912).

Table 1913. Compounds of formula I.A, wherein R² is R²-15.551(=formula 1.1913).

Table 1914. Compounds of formula I.A, wherein R² is R²-15.552(=formula 1.1914).

Table 1915. Compounds of formula I.A, wherein R² is R²-15.553(=formula 1.1915).

Table 1916. Compounds of formula I.A, wherein R² is R²-15.554(=formula 1.1916).

Table 1917. Compounds of formula I.A, wherein R² is R²-15.555(=formula 1.1917).

Table 1918. Compounds of formula I.A, wherein R² is R²-15.556(=formula 1.1918).

Table 1919. Compounds of formula I.A, wherein R² is R²-15.557(=formula 1.1919).

Table 1920. Compounds of formula I.A, wherein R² is R²-15.558(=formula 1.1920).

Table 1921. Compounds of formula I.A, wherein R² is R²-15.559(=formula 1.1921).

Table 1922. Compounds of formula I.A, wherein R² is R²-15.560(=formula 1.1922).

Table 1923. Compounds of formula I.A, wherein R² is R²-15.561(=formula 1.1923).

Table 1924. Compounds of formula I.A, wherein R² is R²-15.562(=formula 1.1924).

Table 1925. Compounds of formula I.A, wherein R² is R²-15.563(=formula 1.1925).

Table 1926. Compounds of formula I.A, wherein R² is R²-15.564(=formula 1.1926).

Table 1927. Compounds of formula I.A, wherein R² is R²-15.565(=formula 1.1927).

Table 1928. Compounds of formula I.A, wherein R² is R²-15.566(=formula 1.1928).

Table 1929. Compounds of formula I.A, wherein R² is R²-15.567(=formula 1.1929).

Table 1930. Compounds of formula I.A, wherein R² is R²-15.568(=formula 1.1930).

Table 1931. Compounds of formula I.A, wherein R² is R²-15.569(=formula 1.1931).

Table 1932. Compounds of formula I.A, wherein R² is R²-15.570(=formula 1.1932).

Table 1933. Compounds of formula I.A, wherein R² is R²-15.571(=formula 1.1933).

Table 1934. Compounds of formula I.A, wherein R² is R²-15.572(=formula 1.1934).

Table 1935. Compounds of formula I.A, wherein R² is R²-15.573(=formula 1.1935).

Table 1936. Compounds of formula I.A, wherein R² is R²-15.574(=formula 1.1936).

Table 1937. Compounds of formula I.A, wherein R² is R²-15.575(=formula 1.1937).

Table 1938. Compounds of formula I.A, wherein R² is R²-15.576(=formula 1.1938).

Table 1939. Compounds of formula I.A, wherein R² is R²-15.577(=formula 1.1939).

Table 1940. Compounds of formula I.A, wherein R² is R²-15.578(=formula 1.1940).

Table 1941. Compounds of formula I.A, wherein R² is R²-15.579(=formula 1.1941).

Table 1942. Compounds of formula I.A, wherein R² is R²-15.580(=formula 1.1942).

Table 1943. Compounds of formula I.A, wherein R² is R²-15.581(=formula 1.1943).

Table 1944. Compounds of formula I.A, wherein R² is R²-15.582(=formula 1.1944).

Table 1945. Compounds of formula I.A, wherein R² is R²-15.583(=formula 1.1945).

Table 1946. Compounds of formula I.A, wherein R² is R²-15.584(=formula 1.1946).

Table 1947. Compounds of formula I.A, wherein R² is R²-15.585(=formula 1.1947).

Table 1948. Compounds of formula I.A, wherein R² is R²-15.586(=formula 1.1948).

Table 1949. Compounds of formula I.A, wherein R² is R²-15.587(=formula 1.1949).

Table 1950. Compounds of formula I.A, wherein R² is R²-15.588(=formula 1.1950).

Table 1951. Compounds of formula I.A, wherein R² is R²-15.589(=formula 1.1951).

Table 1952. Compounds of formula I.A, wherein R² is R²-15.590(=formula 1.1952).

Table 1953. Compounds of formula I.A, wherein R² is R²-15.591(=formula 1.1953).

Table 1954. Compounds of formula I.A, wherein R² is R²-15.592(=formula 1.1954).

Table 1955. Compounds of formula I.A, wherein R² is R²-15.593(=formula 1.1955).

Table 1956. Compounds of formula I.A, wherein R² is R²-15.594(=formula 1.1956).

Table 1957. Compounds of formula I.A, wherein R² is R²-15.595(=formula 1.1957).

Table 1958. Compounds of formula I.A, wherein R² is R²-15.596(=formula 1.1958).

Table 1959. Compounds of formula I.A, wherein R² is R²-15.597(=formula 1.1959).

Table 1960. Compounds of formula I.A, wherein R² is R²-15.598(=formula 1.1960).

Table 1961. Compounds of formula I.A, wherein R² is R²-15.599(=formula 1.1961).

Table 1962. Compounds of formula I.A, wherein R² is R²-15.600(=formula 1.1962).

Table 1963. Compounds of formula I.A, wherein R² is R²-15.601(=formula 1.1963).

Table 1964. Compounds of formula I.A, wherein R² is R²-15.602(=formula 1.1964).

Table 1965. Compounds of formula I.A, wherein R² is R²-15.603(=formula 1.1965).

Table 1966. Compounds of formula I.A, wherein R² is R²-15.604(=formula 1.1966).

Table 1967. Compounds of formula I.A, wherein R² is R²-15.605(=formula 1.1967).

Table 1968. Compounds of formula I.A, wherein R² is R²-15.606(=formula 1.1968).

Table 1969. Compounds of formula I.A, wherein R² is R²-15.607(=formula 1.1969).

Table 1970. Compounds of formula I.A, wherein R² is R²-15.608(=formula 1.1970).

Table 1971. Compounds of formula I.A, wherein R² is R²-15.609(=formula 1.1971).

Table 1972. Compounds of formula I.A, wherein R² is R²-15.610(=formula 1.1972).

Table 1973. Compounds of formula I.A, wherein R² is R²-15.611(=formula 1.1973).

Table 1974. Compounds of formula I.A, wherein R² is R²-15.612(=formula 1.1974).

Table 1975. Compounds of formula I.A, wherein R² is R²-15.613(=formula 1.1975).

Table 1976. Compounds of formula I.A, wherein R² is R²-15.614(=formula 1.1976).

Table 1977. Compounds of formula I.A, wherein R² is R²-15.615(=formula 1.1977).

Table 1978. Compounds of formula I.A, wherein R² is R²-15.616(=formula 1.1978).

Table 1979. Compounds of formula I.A, wherein R² is R²-15.617(=formula 1.1979).

Table 1980. Compounds of formula I.A, wherein R² is R²-15.618(=formula 1.1980).

Table 1981. Compounds of formula I.A, wherein R² is R²-15.619(=formula 1.1981).

Table 1982. Compounds of formula I.A, wherein R² is R²-15.620(=formula 1.1982).

Table 1983. Compounds of formula I.A, wherein R² is R²-15.621(=formula 1.1983).

Table 1984. Compounds of formula I.A, wherein R² is R²-15.622(=formula 1.1984).

Table 1985. Compounds of formula I.A, wherein R² is R²-15.623(=formula 1.1985).

Table 1986. Compounds of formula I.A, wherein R² is R²-15.624(=formula 1.1986).

Table 1987. Compounds of formula I.A, wherein R² is R²-15.625(=formula 1.1987).

Table 1988. Compounds of formula I.A, wherein R² is R²-15.626(=formula 1.1988).

Table 1989. Compounds of formula I.A, wherein R² is R²-15.627(=formula 1.1989).

Table 1990. Compounds of formula I.A, wherein R² is R²-15.628(=formula 1.1990).

Table 1991. Compounds of formula I.A, wherein R² is R²-15.629(=formula 1.1991).

Table 1992. Compounds of formula I.A, wherein R² is R²-15.630(=formula 1.1992).

Table 1993. Compounds of formula I.A, wherein R² is R²-15.631(=formula 1.1993).

Table 1994. Compounds of formula I.A, wherein R² is R²-15.632(=formula 1.1994).

Table 1995. Compounds of formula I.A, wherein R² is R²-15.633(=formula 1.1995).

Table 1996. Compounds of formula I.A, wherein R² is R²-15.634(=formula 1.1996).

Table 1997. Compounds of formula I.A, wherein R² is R²-15.635(=formula 1.1997).

Table 1998. Compounds of formula I.A, wherein R² is R²-15.636(=formula 1.1998).

Table 1999. Compounds of formula I.A, wherein R² is R²-15.637(=formula 1.1999).

Table 2000. Compounds of formula I.A, wherein R² is R²-15.638(=formula 1.2000).

Table 2001. Compounds of formula I.A, wherein R² is R²-15.639(=formula 1.2001).

Table 2002. Compounds of formula I.A, wherein R² is R²-15.640(=formula 1.2002).

Table 2003. Compounds of formula I.A, wherein R² is R²-15.641(=formula 1.2003).

Table 2004. Compounds of formula I.A, wherein R² is R²-15.642(=formula 1.2004).

Table 2005. Compounds of formula I.A, wherein R² is R²-15.643(=formula 1.2005).

Table 2006. Compounds of formula I.A, wherein R² is R²-15.644(=formula 1.2006).

Table 2007. Compounds of formula I.A, wherein R² is R²-15.645(=formula 1.2007).

Table 2008. Compounds of formula I.A, wherein R² is R²-15.646(=formula 1.2008).

Table 2009. Compounds of formula I.A, wherein R² is R²-15.647(=formula 1.2009).

Table 2010. Compounds of formula I.A, wherein R² is R²-15.648(=formula 1.2010).

Table 2011. Compounds of formula I.A, wherein R² is R²-15.649(=formula 1.2011).

Table 2012. Compounds of formula I.A, wherein R² is R²-15.650(=formula 1.2012).

Table 2013. Compounds of formula I.A, wherein R² is R²-15.651(=formula 1.2013).

Table 2014. Compounds of formula I.A, wherein R² is R²-15.652(=formula 1.2014).

Table 2015. Compounds of formula I.A, wherein R² is R²-15.653(=formula 1.2015).

Table 2016. Compounds of formula I.A, wherein R² is R²-15.654(=formula 1.2016).

Table 2017. Compounds of formula I.A, wherein R² is R²-15.655(=formula 1.2017).

Table 2018. Compounds of formula I.A, wherein R² is R²-15.656(=formula 1.2018).

Table 2019. Compounds of formula I.A, wherein R² is R²-15.657(=formula 1.2019).

Table 2020. Compounds of formula I.A, wherein R² is R²-15.658(=formula 1.2020).

Table 2021. Compounds of formula I.A, wherein R² is R²-15.659(=formula 1.2021).

Table 2022. Compounds of formula I.A, wherein R² is R²-15.660(=formula 1.2022).

Table 2023. Compounds of formula I.A, wherein R² is R²-15.661(=formula 1.2023).

Table 2024. Compounds of formula I.A, wherein R² is R²-15.662(=formula 1.2024).

Table 2025. Compounds of formula I.A, wherein R² is R²-15.663(=formula 1.2025).

Table 2026. Compounds of formula I.A, wherein R² is R²-15.664(=formula 1.2026).

Table 2027. Compounds of formula I.A, wherein R² is R²-15.665(=formula 1.2027).

Table 2028. Compounds of formula I.A, wherein R² is R²-15.666(=formula 1.2028).

Table 2029. Compounds of formula I.A, wherein R² is R²-15.667(=formula 1.2029).

Table 2030. Compounds of formula I.A, wherein R² is R²-15.668(=formula 1.2030).

Table 2031. Compounds of formula I.A, wherein R² is R²-15.669(=formula 1.2031).

Table 2032. Compounds of formula I.A, wherein R² is R²-15.670(=formula 1.2032).

Table 2033. Compounds of formula I.A, wherein R² is R²-15.671(=formula 1.2033).

Table 2034. Compounds of formula I.A, wherein R² is R²-15.672(=formula 1.2034).

Table 2035. Compounds of formula I.A, wherein R² is R²-15.673(=formula 1.2035).

Table 2036. Compounds of formula I.A, wherein R² is R²-15.674(=formula 1.2036).

Table 2037. Compounds of formula I.A, wherein R² is R²-15.675(=formula 1.2037).

Table 2038. Compounds of formula I.A, wherein R² is R²-15.676(=formula 1.2038).

Table 2039. Compounds of formula I.A, wherein R² is R²-9.1(=formula 1.2039).

Table 2040. Compounds of formula I.A, wherein R² is R²-9.2(=formula 1.2040).

Table 2041. Compounds of formula I.A, wherein R² is R²-9.3(=formula 1.2041).

Table 2042. Compounds of formula I.A, wherein R² is R²-9.4(=formula 1.2042).

Table 2043. Compounds of formula I.A, wherein R² is R²-9.5(=formula 1.2043).

Table 2044. Compounds of formula I.A, wherein R² is R²-9.6(=formula 1.2044).

Table 2045. Compounds of formula I.A, wherein R² is R²-9.7(=formula 1.2045).

Table 2046. Compounds of formula I.A, wherein R² is R²-9.8(=formula 1.2046).

Table 2047. Compounds of formula I.A, wherein R² is R²-9.9(=formula 1.2047).

Table 2048. Compounds of formula I.A, wherein R² is R²-9.10(=formula 1.2048).

Table 2049. Compounds of formula I.A, wherein R² is R²-9.11(=formula 1.2049).

Table 2050. Compounds of formula I.A, wherein R² is R²-9.12(=formula 1.2050).

Table 2051. Compounds of formula I.A, wherein R² is R²-9.13(=formula 1.2051).

Table 2052. Compounds of formula I.A, wherein R² is R²-9.14(=formula 1.2052).

Table 2053. Compounds of formula I.A, wherein R² is R²-9.15(=formula 1.2053).

TABLE A1

| Line | R¹ | R³ | m | R⁴ |
|---|---|---|---|---|
| I-281 | c-C₃H₅ | CH₃ | 0 | — |
| I-282 | c-C₃H₅ | OCH₃ | 0 | — |
| I-283 | c-C₃H₅ | Cl | 0 | — |
| I-284 | c-C₃H₅ | Br | 0 | — |
| I-285 | c-C₃H₅ | CHF₂ | 0 | — |
| I-286 | c-C₃H₅ | F | 0 | — |
| I-287 | c-C₃H₅ | I | 0 | — |
| I-288 | c-C₃H₅ | CH₃ | 1 | 4″-F |
| I-289 | c-C₃H₅ | OCH₃ | 1 | 4″-F |
| I-290 | c-C₃H₅ | Cl | 1 | 4″-F |
| I-291 | c-C₃H₅ | Br | 1 | 4″-F |
| I-292 | c-C₃H₅ | CHF₂ | 1 | 4″-F |
| I-293 | c-C₃H₅ | F | 1 | 4″-F |
| I-294 | c-C₃H₅ | I | 1 | 4″-F |
| I-295 | c-C₄H₇ | CH₃ | 0 | — |
| I-296 | c-C₄H₇ | OCH₃ | 0 | — |
| I-297 | c-C₄H₇ | Cl | 0 | — |
| I-298 | c-C₄H₇ | Br | 0 | — |
| I-299 | c-C₄H₇ | CHF₂ | 0 | — |
| I-300 | c-C₄H₇ | F | 0 | — |
| I-301 | c-C₄H₇ | I | 0 | — |
| I-302 | c-C₄H₇ | CH₃ | 1 | 4″-F |
| I-303 | c-C₄H₇ | OCH₃ | 1 | 4″-F |
| I-304 | c-C₄H₇ | Cl | 1 | 4″-F |
| I-305 | c-C₄H₇ | Br | 1 | 4″-F |
| I-306 | c-C₄H₇ | CHF₂ | 1 | 4″-F |
| I-307 | c-C₄H₇ | F | 1 | 4″-F |
| I-308 | c-C₄H₇ | I | 1 | 4″-F |
| I-309 | C₂H₅ | CH₃ | 0 | — |
| I-310 | C₂H₅ | OCH₃ | 0 | — |
| I-311 | C₂H₅ | Cl | 0 | — |
| I-312 | C₂H₅ | Br | 0 | — |
| I-313 | C₂H₅ | CHF₂ | 0 | — |
| I-314 | C₂H₅ | F | 0 | — |
| I-315 | C₂H₅ | I | 0 | — |
| I-316 | C₂H₅ | CH₃ | 1 | 4″-F |
| I-317 | C₂H₅ | OCH₃ | 1 | 4″-F |
| I-318 | C₂H₅ | Cl | 1 | 4″-F |
| I-319 | C₂H₅ | Br | 1 | 4″-F |
| I-320 | C₂H₅ | CHF₂ | 1 | 4″-F |
| I-321 | C₂H₅ | F | 1 | 4″-F |
| I-322 | C₂H₅ | I | 1 | 4″-F |
| I-323 | OCH₃ | CH₃ | 0 | — |
| I-324 | OCH₃ | OCH₃ | 0 | — |
| I-325 | OCH₃ | Cl | 0 | — |
| I-326 | OCH₃ | Br | 0 | — |
| I-327 | OCH₃ | CHF₂ | 0 | — |
| I-328 | OCH₃ | F | 0 | — |
| I-329 | OCH₃ | I | 0 | — |
| I-330 | OCH₃ | CH₃ | 1 | 4″-F |
| I-331 | OCH₃ | OCH₃ | 1 | 4″-F |
| I-332 | OCH₃ | Cl | 1 | 4″-F |
| I-333 | OCH₃ | Br | 1 | 4″-F |
| I-334 | OCH₃ | CHF₂ | 1 | 4″-F |
| I-335 | OCH₃ | F | 1 | 4″-F |
| I-336 | OCH₃ | I | 1 | 4″-F |

The specific number for each single compound is deductible as follows:

Compound 1.27.I-317 e.g. comprises the compound of formula 1.27 from Table 27 and line I-317 from Table A1;

According to another particularly preferred embodiment of the compounds of formula I and their use as herbicide, compounds of the invention are the compounds of formulae I that are compiled in tables 2055 to 4082, wherein the meaning for the combination of variables R¹, R³, m, and R⁴ for each individual compound of tables 2055 to 4082 corresponds to each line of Table A2. Each of the groups mentioned for a substituent in the tables is furthermore per se, independently of the combination in which it is mentioned, a particularly preferred aspect of the substituent in question. The term “formula” used in below tables 2055 to 4082 denotes “compounds of formula”.

Table 2055. Compounds of formula I.C, wherein R² is R²-9.17(=formula 1.2055).

Table 2056. Compounds of formula I.C, wherein R² is R²-9.18(=formula 1.2056).

Table 2057. Compounds of formula I.C, wherein R² is R²-9.19(=formula 1.2057).

Table 2058. Compounds of formula I.C, wherein R² is R²-9.20(=formula 1.2058).

Table 2059. Compounds of formula I.C, wherein R² is R²-9.21(=formula 1.2059).

Table 2060. Compounds of formula I.C, wherein R² is R²-9.22(=formula 1.2060).

Table 2061. Compounds of formula I.C, wherein R² is R²-9.23(=formula 1.2061).

Table 2062. Compounds of formula I.C, wherein R² is R²-9.24(=formula 1.2062).

Table 2063. Compounds of formula I.C, wherein R² is R²-9.25(=formula 1.2063).

Table 2064. Compounds of formula I.C, wherein R² is R²-9.26(=formula 1.2064).

Table 2065. Compounds of formula I.C, wherein R² is R²-9.27(=formula 1.2065).

Table 2066. Compounds of formula I.C, wherein R² is R²-9.28(=formula 1.2066).

Table 2067. Compounds of formula I.C, wherein R² is R²-9.29(=formula 1.2067).

Table 2068. Compounds of formula I.C, wherein R² is R²-9.30(=formula 1.2068).

Table 2069. Compounds of formula I.C, wherein R² is R²-9.31(=formula 1.2069).

Table 2070. Compounds of formula I.C, wherein R² is R²-9.32(=formula 1.2070).

Table 2071. Compounds of formula I.C, wherein R² is R²-9.33(=formula 1.2071).

Table 2072. Compounds of formula I.C, wherein R² is R²-9.34(=formula 1.2072).

Table 2073. Compounds of formula I.C, wherein R² is R²-9.35(=formula 1.2073).

Table 2074. Compounds of formula I.C, wherein R² is R²-9.36(=formula 1.2074).

Table 2075. Compounds of formula I.C, wherein R² is R²-9.37(=formula 1.2075).

Table 2076. Compounds of formula I.C, wherein R² is R²-9.38(=formula 1.2076).

Table 2077. Compounds of formula I.C, wherein R² is R²-9.39(=formula 1.2077).

Table 2078. Compounds of formula I.C, wherein R² is R²-9.40(=formula 1.2078).

Table 2079. Compounds of formula I.C, wherein R² is R²-9.41(=formula 1.2079).

Table 2080. Compounds of formula I.C, wherein R² is R²-9.42(=formula 1.2080).

Table 2081. Compounds of formula I.C, wherein R² is R²-9.43(=formula 1.2081).

Table 2082. Compounds of formula I.C, wherein R² is R²-9.44(=formula 1.2082).

Table 2083. Compounds of formula I.C, wherein R² is R²-9.45(=formula 1.2083).

Table 2084. Compounds of formula I.C, wherein R² is R²-9.46(=formula 1.2084).

Table 2085. Compounds of formula I.C, wherein R² is R²-9.47(=formula 1.2085).

Table 2086. Compounds of formula I.C, wherein R² is R²-9.48(=formula 1.2086).

Table 2087. Compounds of formula I.C, wherein R² is R²-9.49(=formula 1.2087).

Table 2088. Compounds of formula I.C, wherein R² is R²-9.50(=formula 1.2088).

Table 2089. Compounds of formula I.C, wherein R² is R²-9.51(=formula 1.2089).

Table 2090. Compounds of formula I.C, wherein R² is R²-9.52(=formula 1.2090).

Table 2091. Compounds of formula I.C, wherein R² is R²-9.53(=formula 1.2091).

Table 2092. Compounds of formula I.C, wherein R² is R²-9.54(=formula 1.2092).

Table 2093. Compounds of formula I.C, wherein R² is R²-9.55(=formula 1.2093).

Table 2094. Compounds of formula I.C, wherein R² is R²-9.56(=formula 1.2094).

Table 2095. Compounds of formula I.C, wherein R² is R²-9.57(=formula 1.2095).

Table 2096. Compounds of formula I.C, wherein R² is R²-9.58(=formula 1.2096).

Table 2097. Compounds of formula I.C, wherein R² is R²-9.59(=formula 1.2097).

Table 2098. Compounds of formula I.C, wherein R² is R²-9.60(=formula 1.2098).

Table 2099. Compounds of formula I.C, wherein R² is R²-9.61(=formula 1.2099).

Table 2100. Compounds of formula I.C, wherein R² is R²-9.62(=formula 1.2100).

Table 2101. Compounds of formula I.C, wherein R² is R²-9.63(=formula 1.2101).

Table 2102. Compounds of formula I.C, wherein R² is R²-9.64(=formula 1.2102).

Table 2103. Compounds of formula I.C, wherein R² is R²-9.65(=formula 1.2103).

Table 2104. Compounds of formula I.C, wherein R² is R²-9.66(=formula 1.2104).

Table 2105. Compounds of formula I.C, wherein R² is R²-9.67(=formula 1.2105).

Table 2106. Compounds of formula I.C, wherein R² is R²-9.68(=formula 1.2106).

Table 2107. Compounds of formula I.C, wherein R² is R²-9.69(=formula 1.2107).

Table 2108. Compounds of formula I.C, wherein R² is R²-9.70(=formula 1.2108).

Table 2109. Compounds of formula I.C, wherein R² is R²-9.71(=formula 1.2109).

Table 2110. Compounds of formula I.C, wherein R² is R²-9.72(=formula 1.2110).

Table 2111. Compounds of formula I.C, wherein R² is R²-9.73(=formula 1.2111).

Table 2112. Compounds of formula I.C, wherein R² is R²-9.74(=formula 1.2112).

Table 2113. Compounds of formula I.C, wherein R² is R²-9.75(=formula 1.2113).

Table 2114. Compounds of formula I.C, wherein R² is R²-9.76(=formula 1.2114).

Table 2115. Compounds of formula I.C, wherein R² is R²-9.77(=formula 1.2115).

Table 2116. Compounds of formula I.C, wherein R² is R²-9.78(=formula 1.2116).

Table 2117. Compounds of formula I.C, wherein R² is R²-9.79(=formula 1.2117).

Table 2118. Compounds of formula I.C, wherein R² is R²-9.80(=formula 1.2118).

Table 2119. Compounds of formula I.C, wherein R² is R²-9.81(=formula 1.2119).

Table 2120. Compounds of formula I.C, wherein R² is R²-9.82(=formula 1.2120).

Table 2121. Compounds of formula I.C, wherein R² is R²-9.83(=formula 1.2121).

Table 2122. Compounds of formula I.C, wherein R² is R²-9.84(=formula 1.2122).

Table 2123. Compounds of formula I.C, wherein R² is R²-9.85(=formula 1.2123).

Table 2124. Compounds of formula I.C, wherein R² is R²-9.86(=formula 1.2124).

Table 2125. Compounds of formula I.C, wherein R² is R²-9.87(=formula 1.2125).

Table 2126. Compounds of formula I.C, wherein R² is R²-9.88(=formula 1.2126).

Table 2127. Compounds of formula I.C, wherein R² is R²-9.89(=formula 1.2127).

Table 2128. Compounds of formula I.C, wherein R² is R²-9.90(=formula 1.2128).

Table 2129. Compounds of formula I.C, wherein R² is R²-9.91(=formula 1.2129).

Table 2130. Compounds of formula I.C, wherein R² is R²-9.92(=formula 1.2130).

Table 2131. Compounds of formula I.C, wherein R² is R²-9.93(=formula 1.2131).

Table 2132. Compounds of formula I.C, wherein R² is R²-9.94(=formula 1.2132).

Table 2133. Compounds of formula I.C, wherein R² is R²-9.95(=formula 1.2133).

Table 2134. Compounds of formula I.C, wherein R² is R²-9.96(=formula 1.2134).

Table 2135. Compounds of formula I.C, wherein R² is R²-9.97(=formula 1.2135).

Table 2136. Compounds of formula I.C, wherein R² is R²-9.98(=formula 1.2136).

Table 2137. Compounds of formula I.C, wherein R² is R²-9.99(=formula 1.2137).

Table 2138. Compounds of formula I.C, wherein R² is R²-9.100(=formula 1.2138).

Table 2139. Compounds of formula I.C, wherein R² is R²-9.101(=formula 1.2139).

Table 2140. Compounds of formula I.C, wherein R² is R²-9.102(=formula 1.2140).

Table 2141. Compounds of formula I.C, wherein R² is R²-9.103(=formula 1.2141).

Table 2142. Compounds of formula I.C, wherein R² is R²-9.104(=formula 1.2142).

Table 2143. Compounds of formula I.C, wherein R² is R²-9.105(=formula 1.2143).

Table 2144. Compounds of formula I.C, wherein R² is R²-9.106(=formula 1.2144).

Table 2145. Compounds of formula I.C, wherein R² is R²-9.107(=formula 1.2145).

Table 2146. Compounds of formula I.C, wherein R² is R²-9.108(=formula 1.2146).

Table 2147. Compounds of formula I.C, wherein R² is R²-9.109(=formula 1.2147).

Table 2148. Compounds of formula I.C, wherein R² is R²-9.110(=formula 1.2148).

Table 2149. Compounds of formula I.C, wherein R² is R²-9.111(=formula 1.2149).

Table 2150. Compounds of formula I.C, wherein R² is R²-9.112(=formula 1.2150).

Table 2151. Compounds of formula I.C, wherein R² is R²-9.113(=formula 1.2151).

Table 2152. Compounds of formula I.C, wherein R² is R²-9.114(=formula 1.2152).

Table 2153. Compounds of formula I.C, wherein R² is R²-9.115(=formula 1.2153).

Table 2154. Compounds of formula I.C, wherein R² is R²-9.116(=formula 1.2154).

Table 2155. Compounds of formula I.C, wherein R² is R²-9.117(=formula 1.2155).

Table 2156. Compounds of formula I.C, wherein R² is R²-9.118(=formula 1.2156).

Table 2157. Compounds of formula I.C, wherein R² is R²-9.119(=formula 1.2157).

Table 2158. Compounds of formula I.C, wherein R² is R²-9.120(=formula 1.2158).

Table 2159. Compounds of formula I.C, wherein R² is R²-9.121(=formula 1.2159).

Table 2160. Compounds of formula I.C, wherein R² is R²-9.122(=formula 1.2160).

Table 2161. Compounds of formula I.C, wherein R² is R²-9.123(=formula 1.2161).

Table 2162. Compounds of formula I.C, wherein R² is R²-9.124(=formula 1.2162).

Table 2163. Compounds of formula I.C, wherein R² is R²-9.125(=formula 1.2163).

Table 2164. Compounds of formula I.C, wherein R² is R²-9.126(=formula 1.2164).

Table 2165. Compounds of formula I.C, wherein R² is R²-9.127(=formula 1.2165).

Table 2166. Compounds of formula I.C, wherein R² is R²-9.128(=formula 1.2166).

Table 2167. Compounds of formula I.C, wherein R² is R²-9.129(=formula 1.2167).

Table 2168. Compounds of formula I.C, wherein R² is R²-9.130(=formula 1.2168).

Table 2169. Compounds of formula I.C, wherein R² is R²-9.131(=formula 1.2169).

Table 2170. Compounds of formula I.C, wherein R² is R²-9.132(=formula 1.2170).

Table 2171. Compounds of formula I.C, wherein R² is R²-9.133(=formula 1.2171).

Table 2172. Compounds of formula I.C, wherein R² is R²-9.134(=formula 1.2172).

Table 2173. Compounds of formula I.C, wherein R² is R²-9.135(=formula 1.2173).

Table 2174. Compounds of formula I.C, wherein R² is R²-9.136(=formula 1.2174).

Table 2175. Compounds of formula I.C, wherein R² is R²-9.137(=formula 1.2175).

Table 2176. Compounds of formula I.C, wherein R² is R²-9.138(=formula 1.2176).

Table 2177. Compounds of formula I.C, wherein R² is R²-9.139(=formula 1.2177).

Table 2178. Compounds of formula I.C, wherein R² is R²-9.140(=formula 1.2178).

Table 2179. Compounds of formula I.C, wherein R² is R²-9.141(=formula 1.2179).

Table 2180. Compounds of formula I.C, wherein R² is R²-9.142(=formula 1.2180).

Table 2181. Compounds of formula I.C, wherein R² is R²-9.143(=formula 1.2181).

Table 2182. Compounds of formula I.C, wherein R² is R²-9.144(=formula 1.2182).

Table 2183. Compounds of formula I.C, wherein R² is R²-9.145(=formula 1.2183).

Table 2184. Compounds of formula I.C, wherein R² is R²-9.146(=formula 1.2184).

Table 2185. Compounds of formula I.C, wherein R² is R²-9.147(=formula 1.2185).

Table 2186. Compounds of formula I.C, wherein R² is R²-9.148(=formula 1.2186).

Table 2187. Compounds of formula I.C, wherein R² is R²-9.149(=formula 1.2187).

Table 2188. Compounds of formula I.C, wherein R² is R²-9.150(=formula 1.2188).

Table 2189. Compounds of formula I.C, wherein R² is R²-9.151(=formula 1.2189).

Table 2190. Compounds of formula I.C, wherein R² is R²-9.152(=formula 1.2190).

Table 2191. Compounds of formula I.C, wherein R² is R²-9.153(=formula 1.2191).

Table 2192. Compounds of formula I.C, wherein R² is R²-9.154(=formula 1.2192).

Table 2193. Compounds of formula I.C, wherein R² is R²-9.155(=formula 1.2193).

Table 2194. Compounds of formula I.C, wherein R² is R²-9.156(=formula 1.2194).

Table 2195. Compounds of formula I.C, wherein R² is R²-9.157(=formula 1.2195).

Table 2196. Compounds of formula I.C, wherein R² is R²-9.158(=formula 1.2196).

Table 2197. Compounds of formula I.C, wherein R² is R²-9.159(=formula 1.2197).

Table 2198. Compounds of formula I.C, wherein R² is R²-9.160(=formula 1.2198).

Table 2199. Compounds of formula I.C, wherein R² is R²-9.161(=formula 1.2199).

Table 2200. Compounds of formula I.C, wherein R² is R²-9.162(=formula 1.2200).

Table 2201. Compounds of formula I.C, wherein R² is R²-9.163(=formula 1.2201).

Table 2202. Compounds of formula I.C, wherein R² is R²-9.164(=formula 1.2202).

Table 2203. Compounds of formula I.C, wherein R² is R²-9.165(=formula 1.2203).

Table 2204. Compounds of formula I.C, wherein R² is R²-9.166(=formula 1.2204).

Table 2205. Compounds of formula I.C, wherein R² is R²-9.167(=formula 1.2205).

Table 2206. Compounds of formula I.C, wherein R² is R²-9.168(=formula 1.2206).

Table 2207. Compounds of formula I.C, wherein R² is R²-9.169(=formula 1.2207).

Table 2208. Compounds of formula I.C, wherein R² is R²-9.170(=formula 1.2208).

Table 2209. Compounds of formula I.C, wherein R² is R²-9.171(=formula 1.2209).

Table 2210. Compounds of formula I.C, wherein R² is R²-9.172(=formula 1.2210).

Table 2211. Compounds of formula I.C, wherein R² is R²-9.173(=formula 1.2211).

Table 2212. Compounds of formula I.C, wherein R² is R²-9.174(=formula 1.2212).

Table 2213. Compounds of formula I.C, wherein R² is R²-9.175(=formula 1.2213).

Table 2214. Compounds of formula I.C, wherein R² is R²-9.176(=formula 1.2214).

Table 2215. Compounds of formula I.C, wherein R² is R²-9.177(=formula 1.2215).

Table 2216. Compounds of formula I.C, wherein R² is R²-9.178(=formula 1.2216).

Table 2217. Compounds of formula I.C, wherein R² is R²-9.179(=formula 1.2217).

Table 2218. Compounds of formula I.C, wherein R² is R²-9.180(=formula 1.2218).

Table 2219. Compounds of formula I.C, wherein R² is R²-9.181(=formula 1.2219).

Table 2220. Compounds of formula I.C, wherein R² is R²-9.182(=formula 1.2220).

Table 2221. Compounds of formula I.C, wherein R² is R²-9.183(=formula 1.2221).

Table 2222. Compounds of formula I.C, wherein R² is R²-9.184(=formula 1.2222).

Table 2223. Compounds of formula I.C, wherein R² is R²-9.185(=formula 1.2223).

Table 2224. Compounds of formula I.C, wherein R² is R²-9.186(=formula 1.2224).

Table 2225. Compounds of formula I.C, wherein R² is R²-9.187(=formula 1.2225).

Table 2226. Compounds of formula I.C, wherein R² is R²-9.188(=formula 1.2226).

Table 2227. Compounds of formula I.C, wherein R² is R²-9.189(=formula 1.2227).

Table 2228. Compounds of formula I.C, wherein R² is R²-9.190(=formula 1.2228).

Table 2229. Compounds of formula I.C, wherein R² is R²-9.191(=formula 1.2229).

Table 2230. Compounds of formula I.C, wherein R² is R²-9.192(=formula 1.2230).

Table 2231. Compounds of formula I.C, wherein R² is R²-9.193(=formula 1.2231).

Table 2232. Compounds of formula I.C, wherein R² is R²-9.194(=formula 1.2232).

Table 2233. Compounds of formula I.C, wherein R² is R²-9.195(=formula 1.2233).

Table 2234. Compounds of formula I.C, wherein R² is R²-9.196(=formula 1.2234).

Table 2235. Compounds of formula I.C, wherein R² is R²-9.197(=formula 1.2235).

Table 2236. Compounds of formula I.C, wherein R² is R²-9.198(=formula 1.2236).

Table 2237. Compounds of formula I.C, wherein R² is R²-9.199(=formula 1.2237).

Table 2238. Compounds of formula I.C, wherein R² is R²-9.200(=formula 1.2238).

Table 2239. Compounds of formula I.C, wherein R² is R²-9.201(=formula 1.2239).

Table 2240. Compounds of formula I.C, wherein R² is R²-9.202(=formula 1.2240).

Table 2241. Compounds of formula I.C, wherein R² is R²-9.203(=formula 1.2241).

Table 2242. Compounds of formula I.C, wherein R² is R²-9.204(=formula 1.2242).

Table 2243. Compounds of formula I.C, wherein R² is R²-9.205(=formula 1.2243).

Table 2244. Compounds of formula I.C, wherein R² is R²-9.206(=formula 1.2244).

Table 2245. Compounds of formula I.C, wherein R² is R²-9.207(=formula 1.2245).

Table 2246. Compounds of formula I.C, wherein R² is R²-9.208(=formula 1.2246).

Table 2247. Compounds of formula I.C, wherein R² is R²-9.209(=formula 1.2247).

Table 2248. Compounds of formula I.C, wherein R² is R²-9.210(=formula 1.2248).

Table 2249. Compounds of formula I.C, wherein R² is R²-9.211(=formula 1.2249).

Table 2250. Compounds of formula I.C, wherein R² is R²-9.212(=formula 1.2250).

Table 2251. Compounds of formula I.C, wherein R² is R²-9.213(=formula 1.2251).

Table 2252. Compounds of formula I.C, wherein R² is R²-9.214(=formula 1.2252).

Table 2253. Compounds of formula I.C, wherein R² is R²-9.215(=formula 1.2253).

Table 2254. Compounds of formula I.C, wherein R² is R²-9.216(=formula 1.2254).

Table 2255. Compounds of formula I.C, wherein R² is R²-9.217(=formula 1.2255).

Table 2256. Compounds of formula I.C, wherein R² is R²-9.218(=formula 1.2256).

Table 2257. Compounds of formula I.C, wherein R² is R²-9.219(=formula 1.2257).

Table 2258. Compounds of formula I.C, wherein R² is R²-9.220(=formula 1.2258).

Table 2259. Compounds of formula I.C, wherein R² is R²-9.221(=formula 1.2259).

Table 2260. Compounds of formula I.C, wherein R² is R²-9.222(=formula 1.2260).

Table 2261. Compounds of formula I.C, wherein R² is R²-9.223(=formula 1.2261).

Table 2262. Compounds of formula I.C, wherein R² is R²-9.224(=formula 1.2262).

Table 2263. Compounds of formula I.C, wherein R² is R²-9.225(=formula 1.2263).

Table 2264. Compounds of formula I.C, wherein R² is R²-9.226(=formula 1.2264).

Table 2265. Compounds of formula I.C, wherein R² is R²-9.227(=formula 1.2265).

Table 2266. Compounds of formula I.C, wherein R² is R²-9.228(=formula 1.2266).

Table 2267. Compounds of formula I.C, wherein R² is R²-9.229(=formula 1.2267).

Table 2268. Compounds of formula I.C, wherein R² is R²-9.230(=formula 1.2268).

Table 2269. Compounds of formula I.C, wherein R² is R²-9.231(=formula 1.2269).

Table 2270. Compounds of formula I.C, wherein R² is R²-9.232(=formula 1.2270).

Table 2271. Compounds of formula I.C, wherein R² is R²-9.233(=formula 1.2271).

Table 2272. Compounds of formula I.C, wherein R² is R²-9.234(=formula 1.2272).

Table 2273. Compounds of formula I.C, wherein R² is R²-9.235(=formula 1.2273).

Table 2274. Compounds of formula I.C, wherein R² is R²-9.236(=formula 1.2274).

Table 2275. Compounds of formula I.C, wherein R² is R²-9.237(=formula 1.2275).

Table 2276. Compounds of formula I.C, wherein R² is R²-9.238(=formula 1.2276).

Table 2277. Compounds of formula I.C, wherein R² is R²-9.239(=formula 1.2277).

Table 2278. Compounds of formula I.C, wherein R² is R²-9.240(=formula 1.2278).

Table 2279. Compounds of formula I.C, wherein R² is R²-9.241(=formula 1.2279).

Table 2280. Compounds of formula I.C, wherein R² is R²-9.242(=formula 1.2280).

Table 2281. Compounds of formula I.C, wherein R² is R²-9.243 (=formula 1.2281).

Table 2282. Compounds of formula I.C, wherein R² is R²-9.244(=formula 1.2282).

Table 2283. Compounds of formula I.C, wherein R² is R²-9.245(=formula 1.2283).

Table 2284. Compounds of formula I.C, wherein R² is R²-9.246(=formula 1.2284).

Table 2285. Compounds of formula I.C, wherein R² is R²-9.247(=formula 1.2285).

Table 2286. Compounds of formula I.C, wherein R² is R²-9.248(=formula 1.2286).

Table 2287. Compounds of formula I.C, wherein R² is R²-9.249(=formula 1.2287).

Table 2288. Compounds of formula I.C, wherein R² is R²-9.250(=formula 1.2288).

Table 2289. Compounds of formula I.C, wherein R² is R²-9.251(=formula 1.2289).

Table 2290. Compounds of formula I.C, wherein R² is R²-9.252(=formula 1.2290).

Table 2291. Compounds of formula I.C, wherein R² is R²-9.253(=formula 1.2291).

Table 2292. Compounds of formula I.C, wherein R² is R²-9.254(=formula 1.2292).

Table 2293. Compounds of formula I.C, wherein R² is R²-9.255(=formula 1.2293).

Table 2294. Compounds of formula I.C, wherein R² is R²-9.256(=formula 1.2294).

Table 2295. Compounds of formula I.C, wherein R² is R²-9.257(=formula 1.2295).

Table 2296. Compounds of formula I.C, wherein R² is R²-9.258(=formula 1.2296).

Table 2297. Compounds of formula I.C, wherein R² is R²-9.259(=formula 1.2297).

Table 2298. Compounds of formula I.C, wherein R² is R²-9.260(=formula 1.2298).

Table 2299. Compounds of formula I.C, wherein R² is R²-9.261(=formula 1.2299).

Table 2300. Compounds of formula I.C, wherein R² is R²-9.262(=formula 1.2300).

Table 2301. Compounds of formula I.C, wherein R² is R²-9.263(=formula 1.2301).

Table 2302. Compounds of formula I.C, wherein R² is R²-9.264(=formula 1.2302).

Table 2303. Compounds of formula I.C, wherein R² is R²-9.265 (=formula 1.2303).

Table 2304. Compounds of formula I.C, wherein R² is R²-9.266 (=formula 1.2304).

Table 2305. Compounds of formula I.C, wherein R² is R²-9.267(=formula 1.2305).

Table 2306. Compounds of formula I.C, wherein R² is R²-9.268(=formula 1.2306).

Table 2307. Compounds of formula I.C, wherein R² is R²-9.269(=formula 1.2307).

Table 2308. Compounds of formula I.C, wherein R² is R²-9.270 (=formula 1.2308).

Table 2309. Compounds of formula I.C, wherein R² is R²-9.271(=formula 1.2309).

Table 2310. Compounds of formula I.C, wherein R² is R²-9.272(=formula 1.2310).

Table 2311. Compounds of formula I.C, wherein R² is R²-9.273 (=formula 1.2311).

Table 2312. Compounds of formula I.C, wherein R² is R²-9.274 (=formula 1.2312).

Table 2313. Compounds of formula I.C, wherein R² is R²-9.275 (=formula 1.2313).

Table 2314. Compounds of formula I.C, wherein R² is R²-9.276 (=formula 1.2314).

Table 2315. Compounds of formula I.C, wherein R² is R²-9.277(=formula 1.2315).

Table 2316. Compounds of formula I.C, wherein R² is R²-9.278(=formula 1.2316).

Table 2317. Compounds of formula I.C, wherein R² is R²-9.279(=formula 1.2317).

Table 2318. Compounds of formula I.C, wherein R² is R²-9.280(=formula 1.2318).

Table 2319. Compounds of formula I.C, wherein R² is R²-9.281(=formula 1.2319).

Table 2320. Compounds of formula I.C, wherein R² is R²-9.282(=formula 1.2320).

Table 2321. Compounds of formula I.C, wherein R² is R²-9.283(=formula 1.2321).

Table 2322. Compounds of formula I.C, wherein R² is R²-9.284(=formula 1.2322).

Table 2323. Compounds of formula I.C, wherein R² is R²-9.285(=formula 1.2323).

Table 2324. Compounds of formula I.C, wherein R² is R²-9.286(=formula 1.2324).

Table 2325. Compounds of formula I.C, wherein R² is R²-9.287(=formula 1.2325).

Table 2326. Compounds of formula I.C, wherein R² is R²-9.288(=formula 1.2326).

Table 2327. Compounds of formula I.C, wherein R² is R²-9.289(=formula 1.2327).

Table 2328. Compounds of formula I.C, wherein R² is R²-9.290(=formula 1.2328).

Table 2329. Compounds of formula I.C, wherein R² is R²-9.291(=formula 1.2329).

Table 2330. Compounds of formula I.C, wherein R² is R²-9.292(=formula 1.2330).

Table 2331. Compounds of formula I.C, wherein R² is R²-9.293(=formula 1.2331).

Table 2332. Compounds of formula I.C, wherein R² is R²-9.294(=formula 1.2332).

Table 2333. Compounds of formula I.C, wherein R² is R²-9.295(=formula 1.2333).

Table 2334. Compounds of formula I.C, wherein R² is R²-9.296(=formula 1.2334).

Table 2335. Compounds of formula I.C, wherein R² is R²-9.297(=formula 1.2335).

Table 2336. Compounds of formula I.C, wherein R² is R²-9.298(=formula 1.2336).

Table 2337. Compounds of formula I.C, wherein R² is R²-9.299(=formula 1.2337).

Table 2338. Compounds of formula I.C, wherein R² is R²-9.300 (=formula 1.2338).

Table 2339. Compounds of formula I.C, wherein R² is R²-9.301(=formula 1.2339).

Table 2340. Compounds of formula I.C, wherein R² is R²-9.302(=formula 1.2340).

Table 2341. Compounds of formula I.C, wherein R² is R²-9.303(=formula 1.2341).

Table 2342. Compounds of formula I.C, wherein R² is R²-9.304(=formula 1.2342).

Table 2343. Compounds of formula I.C, wherein R² is R²-9.305(=formula 1.2343).

Table 2344. Compounds of formula I.C, wherein R² is R²-9.306(=formula 1.2344).

Table 2345. Compounds of formula I.C, wherein R² is R²-9.307(=formula 1.2345).

Table 2346. Compounds of formula I.C, wherein R² is R²-9.308(=formula 1.2346).

Table 2347. Compounds of formula I.C, wherein R² is R²-9.309(=formula 1.2347).

Table 2348. Compounds of formula I.C, wherein R² is R²-9.310(=formula 1.2348).

Table 2349. Compounds of formula I.C, wherein R² is R²-9.311(=formula 1.2349).

Table 2350. Compounds of formula I.C, wherein R² is R²-9.312(=formula 1.2350).

Table 2351. Compounds of formula I.C, wherein R² is R²-9.313(=formula 1.2351).

Table 2352. Compounds of formula I.C, wherein R² is R²-9.314(=formula 1.2352).

Table 2353. Compounds of formula I.C, wherein R² is R²-9.315(=formula 1.2353).

Table 2354. Compounds of formula I.C, wherein R² is R²-9.316(=formula 1.2354).

Table 2355. Compounds of formula I.C, wherein R² is R²-9.317(=formula 1.2355).

Table 2356. Compounds of formula I.C, wherein R² is R²-9.318(=formula 1.2356).

Table 2357. Compounds of formula I.C, wherein R² is R²-9.319(=formula 1.2357).

Table 2358. Compounds of formula I.C, wherein R² is R²-9.320(=formula 1.2358).

Table 2359. Compounds of formula I.C, wherein R² is R²-9.321(=formula 1.2359).

Table 2360. Compounds of formula I.C, wherein R² is R²-9.322(=formula 1.2360).

Table 2361. Compounds of formula I.C, wherein R² is R²-9.323(=formula 1.2361).

Table 2362. Compounds of formula I.C, wherein R² is R²-9.324(=formula 1.2362).

Table 2363. Compounds of formula I.C, wherein R² is R²-9.325(=formula 1.2363).

Table 2364. Compounds of formula I.C, wherein R² is R²-9.326(=formula 1.2364).

Table 2365. Compounds of formula I.C, wherein R² is R²-9.327(=formula 1.2365).

Table 2366. Compounds of formula I.C, wherein R² is R²-9.328(=formula 1.2366).

Table 2367. Compounds of formula I.C, wherein R² is R²-9.329(=formula 1.2367).

Table 2368. Compounds of formula I.C, wherein R² is R²-9.330(=formula 1.2368).

Table 2369. Compounds of formula I.C, wherein R² is R²-9.331(=formula 1.2369).

Table 2370. Compounds of formula I.C, wherein R² is R²-9.332(=formula 1.2370).

Table 2371. Compounds of formula I.C, wherein R² is R²-9.333(=formula 1.2371).

Table 2372. Compounds of formula I.C, wherein R² is R²-9.334(=formula 1.2372).

Table 2373. Compounds of formula I.C, wherein R² is R²-9.335(=formula 1.2373).

Table 2374. Compounds of formula I.C, wherein R² is R²-9.336(=formula 1.2374).

Table 2375. Compounds of formula I.C, wherein R² is R²-9.334(=formula 1.2375).

Table 2376. Compounds of formula I.C, wherein R² is R²-9.338(=formula 1.2376).

Table 2377. Compounds of formula I.C, wherein R² is R²-9.339(=formula 1.2377).

Table 2378. Compounds of formula I.C, wherein R² is R²-9.340(=formula 1.2378).

Table 2379. Compounds of formula I.C, wherein R² is R²-9.341(=formula 1.2379).

Table 2380. Compounds of formula I.C, wherein R² is R²-9.342(=formula 1.2380).

Table 2381. Compounds of formula I.C, wherein R² is R²-9.343(=formula 1.2381).

Table 2382. Compounds of formula I.C, wherein R² is R²-9.344(=formula 1.2382).

Table 2383. Compounds of formula I.C, wherein R² is R²-9.345(=formula 1.2383).

Table 2384. Compounds of formula I.C, wherein R² is R²-9.346(=formula 1.2384).

Table 2385. Compounds of formula I.C, wherein R² is R²-9.347(=formula 1.2385).

Table 2386. Compounds of formula I.C, wherein R² is R²-9.348(=formula 1.2386).

Table 2387. Compounds of formula I.C, wherein R² is R²-9.349(=formula 1.2387).

Table 2388. Compounds of formula I.C, wherein R² is R²-9.350(=formula 1.2388).

Table 2389. Compounds of formula I.C, wherein R² is R²-9.351(=formula 1.2389).

Table 2390. Compounds of formula I.C, wherein R² is R²-9.352(=formula 1.2390).

Table 2391. Compounds of formula I.C, wherein R² is R²-9.353(=formula 1.2391).

Table 2392. Compounds of formula I.C, wherein R² is R²-9.354(=formula 1.2392).

Table 2393. Compounds of formula I.C, wherein R² is R²-9.355(=formula 1.2393).

Table 2394. Compounds of formula I.C, wherein R² is R²-9.356(=formula 1.2394).

Table 2395. Compounds of formula I.C, wherein R² is R²-9.357(=formula 1.2395).

Table 2396. Compounds of formula I.C, wherein R² is R²-9.358(=formula 1.2396).

Table 2397. Compounds of formula I.C, wherein R² is R²-9.359(=formula 1.2397).

Table 2398. Compounds of formula I.C, wherein R² is R²-9.360(=formula 1.2398).

Table 2399. Compounds of formula I.C, wherein R² is R²-9.361(=formula 1.2399).

Table 2400. Compounds of formula I.C, wherein R² is R²-9.362(=formula 1.2400).

Table 2401. Compounds of formula I.C, wherein R² is R²-9.363(=formula 1.2401).

Table 2402. Compounds of formula I.C, wherein R² is R²-9.364(=formula 1.2402).

Table 2403. Compounds of formula I.C, wherein R² is R²-9.365(=formula 1.2403).

Table 2404. Compounds of formula I.C, wherein R² is R²-9.366(=formula 1.2404).

Table 2405. Compounds of formula I.C, wherein R² is R²-9.367(=formula 1.2405).

Table 2406. Compounds of formula I.C, wherein R² is R²-9.368(=formula 1.2406).

Table 2407. Compounds of formula I.C, wherein R² is R²-9.369(=formula 1.2407).

Table 2408. Compounds of formula I.C, wherein R² is R²-9.370(=formula 1.2408).

Table 2409. Compounds of formula I.C, wherein R² is R²-9.371(=formula 1.2409).

Table 2410. Compounds of formula I.C, wherein R² is R²-9.372(=formula 1.2410).

Table 2411. Compounds of formula I.C, wherein R² is R²-9.373(=formula 1.2411).

Table 2412. Compounds of formula I.C, wherein R² is R²-9.374(=formula 1.2412).

Table 2413. Compounds of formula I.C, wherein R² is R²-9.375(=formula 1.2413).

Table 2414. Compounds of formula I.C, wherein R² is R²-9.376(=formula 1.2414).

Table 2415. Compounds of formula I.C, wherein R² is R²-9.377(=formula 1.2415).

Table 2416. Compounds of formula I.C, wherein R² is R²-9.378(=formula 1.2416).

Table 2417. Compounds of formula I.C, wherein R² is R²-9.379(=formula 1.2417).

Table 2418. Compounds of formula I.C, wherein R² is R²-9.380(=formula 1.2418).

Table 2419. Compounds of formula I.C, wherein R² is R²-9.381(=formula 1.2419).

Table 2420. Compounds of formula I.C, wherein R² is R²-9.382(=formula 1.2420).

Table 2421. Compounds of formula I.C, wherein R² is R²-9.383(=formula 1.2421).

Table 2422. Compounds of formula I.C, wherein R² is R²-9.384(=formula 1.2422).

Table 2423. Compounds of formula I.C, wherein R² is R²-9.385(=formula 1.2423).

Table 2424. Compounds of formula I.C, wherein R² is R²-9.386(=formula 1.2424).

Table 2425. Compounds of formula I.C, wherein R² is R²-9.387(=formula 1.2425).

Table 2426. Compounds of formula I.C, wherein R² is R²-9.388(=formula 1.2426).

Table 2427. Compounds of formula I.C, wherein R² is R²-9.389(=formula 1.2427).

Table 2428. Compounds of formula I.C, wherein R² is R²-9.390(=formula 1.2428).

Table 2429. Compounds of formula I.C, wherein R² is R²-9.391(=formula 1.2429).

Table 2430. Compounds of formula I.C, wherein R² is R²-9.392(=formula 1.2430).

Table 2431. Compounds of formula I.C, wherein R² is R²-9.393(=formula 1.2431).

Table 2432. Compounds of formula I.C, wherein R² is R²-9.394(=formula 1.2432).

Table 2433. Compounds of formula I.C, wherein R² is R²-9.395(=formula 1.2433).

Table 2434. Compounds of formula I.C, wherein R² is R²-9.396(=formula 1.2434).

Table 2435. Compounds of formula I.C, wherein R² is R²-9.397(=formula 1.2435).

Table 2436. Compounds of formula I.C, wherein R² is R²-9.398(=formula 1.2436).

Table 2437. Compounds of formula I.C, wherein R² is R²-9.399(=formula 1.2437).

Table 2438. Compounds of formula I.C, wherein R² is R²-9.400(=formula 1.2438).

Table 2439. Compounds of formula I.C, wherein R² is R²-9.401(=formula 1.2439).

Table 2440. Compounds of formula I.C, wherein R² is R²-9.402(=formula 1.2440).

Table 2441. Compounds of formula I.C, wherein R² is R²-9.403(=formula 1.2441).

Table 2442. Compounds of formula I.C, wherein R² is R²-9.404(=formula 1.2442).

Table 2443. Compounds of formula I.C, wherein R² is R²-9.405(=formula 1.2443).

Table 2444. Compounds of formula I.C, wherein R² is R²-9.406(=formula 1.2444).

Table 2445. Compounds of formula I.C, wherein R² is R²-9.407(=formula 1.2445).

Table 2446. Compounds of formula I.C, wherein R² is R²-9.408(=formula 1.2446).

Table 2447. Compounds of formula I.C, wherein R² is R²-9.409(=formula 1.2447).

Table 2448. Compounds of formula I.C, wherein R² is R²-9.410(=formula 1.2448).

Table 2449. Compounds of formula I.C, wherein R² is R²-9.411(=formula 1.2449).

Table 2450. Compounds of formula I.C, wherein R² is R²-9.412(=formula 1.2450).

Table 2451. Compounds of formula I.C, wherein R² is R²-9.413(=formula 1.2451).

Table 2452. Compounds of formula I.C, wherein R² is R²-9.414(=formula 1.2452).

Table 2453. Compounds of formula I.C, wherein R² is R²-9.415(=formula 1.2453).

Table 2454. Compounds of formula I.C, wherein R² is R²-9.416(=formula 1.2454).

Table 2455. Compounds of formula I.C, wherein R² is R²-9.417(=formula 1.2455).

Table 2456. Compounds of formula I.C, wherein R² is R²-9.418(=formula 1.2456).

Table 2457. Compounds of formula I.C, wherein R² is R²-9.419(=formula 1.2457).

Table 2458. Compounds of formula I.C, wherein R² is R²-9.420(=formula 1.2458).

Table 2459. Compounds of formula I.C, wherein R² is R²-9.421(=formula 1.2459).

Table 2460. Compounds of formula I.C, wherein R² is R²-9.422(=formula 1.2460).

Table 2461. Compounds of formula I.C, wherein R² is R²-9.423(=formula 1.2461).

Table 2462. Compounds of formula I.C, wherein R² is R²-9.424(=formula 1.2462).

Table 2463. Compounds of formula I.C, wherein R² is R²-9.425(=formula 1.2463).

Table 2464. Compounds of formula I.C, wherein R² is R²-9.426(=formula 1.2464).

Table 2465. Compounds of formula I.C, wherein R² is R²-9.427(=formula 1.2465).

Table 2466. Compounds of formula I.C, wherein R² is R²-9.428(=formula 1.2466).

Table 2467. Compounds of formula I.C, wherein R² is R²-9.429(=formula 1.2467).

Table 2468. Compounds of formula I.C, wherein R² is R²-9.430(=formula 1.2468).

Table 2469. Compounds of formula I.C, wherein R² is R²-9.431(=formula 1.2469).

Table 2470. Compounds of formula I.C, wherein R² is R²-9.432(=formula 1.2470).

Table 2471. Compounds of formula I.C, wherein R² is R²-9.433(=formula 1.2471).

Table 2472. Compounds of formula I.C, wherein R² is R²-9.434(=formula 1.2472).

Table 2473. Compounds of formula I.C, wherein R² is R²-9.435(=formula 1.2473).

Table 2474. Compounds of formula I.C, wherein R² is R²-9.436(=formula 1.2474).

Table 2475. Compounds of formula I.C, wherein R² is R²-9.437(=formula 1.2475).

Table 2476. Compounds of formula I.C, wherein R² is R²-9.438(=formula 1.2476).

Table 2477. Compounds of formula I.C, wherein R² is R²-9.439(=formula 1.2477).

Table 2478. Compounds of formula I.C, wherein R² is R²-9.440(=formula 1.2478).

Table 2479. Compounds of formula I.C, wherein R² is R²-9.441(=formula 1.2479).

Table 2480. Compounds of formula I.C, wherein R² is R²-9.442(=formula 1.2480).

Table 2481. Compounds of formula I.C, wherein R² is R²-9.443(=formula 1.2481).

Table 2482. Compounds of formula I.C, wherein R² is R²-9.444(=formula 1.2482).

Table 2483. Compounds of formula I.C, wherein R² is R²-9.445(=formula 1.2483).

Table 2484. Compounds of formula I.C, wherein R² is R²-9.446(=formula 1.2484).

Table 2485. Compounds of formula I.C, wherein R² is R²-9.447(=formula 1.2485).

Table 2486. Compounds of formula I.C, wherein R² is R²-9.448(=formula 1.2486).

Table 2487. Compounds of formula I.C, wherein R² is R²-9.449(=formula 1.2487).

Table 2488. Compounds of formula I.C, wherein R² is R²-9.450(=formula 1.2488).

Table 2489. Compounds of formula I.C, wherein R² is R²-9.451(=formula 1.2489).

Table 2490. Compounds of formula I.C, wherein R² is R²-9.452(=formula 1.2490).

Table 2491. Compounds of formula I.C, wherein R² is R²-9.453(=formula 1.2491).

Table 2492. Compounds of formula I.C, wherein R² is R²-9.454(=formula 1.2492).

Table 2493. Compounds of formula I.C, wherein R² is R²-9.455(=formula 1.2493).

Table 2494. Compounds of formula I.C, wherein R² is R²-9.456(=formula 1.2494).

Table 2495. Compounds of formula I.C, wherein R² is R²-9.457(=formula 1.2495).

Table 2496. Compounds of formula I.C, wherein R² is R²-9.458(=formula 1.2496).

Table 2497. Compounds of formula I.C, wherein R² is R²-9.459(=formula 1.2497).

Table 2498. Compounds of formula I.C, wherein R² is R²-9.460(=formula 1.2498).

Table 2499. Compounds of formula I.C, wherein R² is R²-9.461(=formula 1.2499).

Table 2500. Compounds of formula I.C, wherein R² is R²-9.462(=formula 1.2500).

Table 2501. Compounds of formula I.C, wherein R² is R²-9.463(=formula 1.2501).

Table 2502. Compounds of formula I.C, wherein R² is R²-9.464(=formula 1.2502).

Table 2503. Compounds of formula I.C, wherein R² is R²-9.465(=formula 1.2503).

Table 2504. Compounds of formula I.C, wherein R² is R²-9.466(=formula 1.2504).

Table 2505. Compounds of formula I.C, wherein R² is R²-9.467(=formula 1.2505).

Table 2506. Compounds of formula I.C, wherein R² is R²-9.468(=formula 1.2506).

Table 2507. Compounds of formula I.C, wherein R² is R²-9.469(=formula 1.2507).

Table 2508. Compounds of formula I.C, wherein R² is R²-9.470(=formula 1.2508).

Table 2509. Compounds of formula I.C, wherein R² is R²-9.471(=formula 1.2509).

Table 2510. Compounds of formula I.C, wherein R² is R²-9.472(=formula 1.2510).

Table 2511. Compounds of formula I.C, wherein R² is R²-9.473(=formula 1.2511).

Table 2512. Compounds of formula I.C, wherein R² is R²-9.474(=formula 1.2512).

Table 2513. Compounds of formula I.C, wherein R² is R²-9.475(=formula 1.2513).

Table 2514. Compounds of formula I.C, wherein R² is R²-9.476(=formula 1.2514).

Table 2515. Compounds of formula I.C, wherein R² is R²-9.477(=formula 1.2515).

Table 2516. Compounds of formula I.C, wherein R² is R²-9.478(=formula 1.2516).

Table 2517. Compounds of formula I.C, wherein R² is R²-9.479(=formula 1.2517).

Table 2518. Compounds of formula I.C, wherein R² is R²-9.480(=formula 1.2518).

Table 2519. Compounds of formula I.C, wherein R² is R²-9.481(=formula 1.2519).

Table 2520. Compounds of formula I.C, wherein R² is R²-9.482(=formula 1.2520).

Table 2521. Compounds of formula I.C, wherein R² is R²-9.483(=formula 1.2521).

Table 2522. Compounds of formula I.C, wherein R² is R²-9.484(=formula 1.2522).

Table 2523. Compounds of formula I.C, wherein R² is R²-9.485(=formula 1.2523).

Table 2524. Compounds of formula I.C, wherein R² is R²-9.486(=formula 1.2521).

Table 2525. Compounds of formula I.C, wherein R² is R²-9.487(=formula 1.2525).

Table 2526. Compounds of formula I.C, wherein R² is R²-9.488(=formula 1.2526).

Table 2527. Compounds of formula I.C, wherein R² is R²-9.489(=formula 1.2527).

Table 2528. Compounds of formula I.C, wherein R² is R²-9.490(=formula 1.2528).

Table 2529. Compounds of formula I.C, wherein R² is R²-9.491(=formula 1.2529).

Table 2530. Compounds of formula I.C, wherein R² is R²-9.492 (=formula 1.2530).

Table 2531. Compounds of formula I.C, wherein R² is R²-9.493(=formula 1.2531).

Table 2532. Compounds of formula I.C, wherein R² is R²-9.494(=formula 1.2532).

Table 2533. Compounds of formula I.C, wherein R² is R²-9.495(=formula 1.2533).

Table 2534. Compounds of formula I.C, wherein R² is R²-9.496(=formula 1.2534).

Table 2535. Compounds of formula I.C, wherein R² is R²-9.497(=formula 1.2535).

Table 2536. Compounds of formula I.C, wherein R² is R²-9.498(=formula 1.2536).

Table 2537. Compounds of formula I.C, wherein R² is R²-9.499(=formula 1.2537).

Table 2538. Compounds of formula I.C, wherein R² is R²-9.500(=formula 1.2538).

Table 2539. Compounds of formula I.C, wherein R² is R²-9.501(=formula 1.2539).

Table 2540. Compounds of formula I.C, wherein R² is R²-9.502(=formula 1.2540).

Table 2541. Compounds of formula I.C, wherein R² is R²-9.503(=formula 1.2541).

Table 2542. Compounds of formula I.C, wherein R² is R²-9.504(=formula 1.2542).

Table 2543. Compounds of formula I.C, wherein R² is R²-9.505(=formula 1.2543).

Table 2544. Compounds of formula I.C, wherein R² is R²-9.506(=formula 1.2544).

Table 2545. Compounds of formula I.C, wherein R² is R²-9.507(=formula 1.2545).

Table 2546. Compounds of formula I.C, wherein R² is R²-9.508(=formula 1.2546).

Table 2547. Compounds of formula I.C, wherein R² is R²-9.509(=formula 1.2547).

Table 2548. Compounds of formula I.C, wherein R² is R²-9.510(=formula 1.2548).

Table 2549. Compounds of formula I.C, wherein R² is R²-9.511(=formula 1.2549).

Table 2550. Compounds of formula I.C, wherein R² is R²-9.512 (=formula 1.2550).

Table 2551. Compounds of formula I.C, wherein R² is R²-9.513 (=formula 1.2551).

Table 2552. Compounds of formula I.C, wherein R² is R²-9.514(=formula 1.2552).

Table 2553. Compounds of formula I.C, wherein R² is R²-9.515(=formula 1.2553).

Table 2554. Compounds of formula I.C, wherein R² is R²-9.516(=formula 1.2554).

Table 2555. Compounds of formula I.C, wherein R² is R²-9.517(=formula 1.2555).

Table 2556. Compounds of formula I.C, wherein R² is R²-9.518(=formula 1.2556).

Table 2557. Compounds of formula I.C, wherein R² is R²-9.519(=formula 1.2557).

Table 2558. Compounds of formula I.C, wherein R² is R²-9.520(=formula 1.2558).

Table 2559. Compounds of formula I.C, wherein R² is R²-9.521(=formula 1.2559).

Table 2560. Compounds of formula I.C, wherein R² is R²-9.522(=formula 1.2560).

Table 2561. Compounds of formula I.C, wherein R² is R²-9.523(=formula 1.2561).

Table 2562. Compounds of formula I.C, wherein R² is R²-9.524(=formula 1.2562).

Table 2563. Compounds of formula I.C, wherein R² is R²-9.525(=formula 1.2563).

Table 2564. Compounds of formula I.C, wherein R² is R²-9.526(=formula 1.2564).

Table 2565. Compounds of formula I.C, wherein R² is R²-9.527(=formula 1.2565).

Table 2566. Compounds of formula I.C, wherein R² is R²-9.528(=formula 1.2566).

Table 2567. Compounds of formula I.C, wherein R² is R²-9.529(=formula 1.2567).

Table 2568. Compounds of formula I.C, wherein R² is R²-9.530(=formula 1.2568).

Table 2569. Compounds of formula I.C, wherein R² is R²-9.531(=formula 1.2569).

Table 2570. Compounds of formula I.C, wherein R² is R²-9.532(=formula 1.2570).

Table 2571. Compounds of formula I.C, wherein R² is R²-9.533(=formula 1.2571).

Table 2572. Compounds of formula I.C, wherein R² is R²-9.534(=formula 1.2572).

Table 2573. Compounds of formula I.C, wherein R² is R²-9.535(=formula 1.2573).

Table 2574. Compounds of formula I.C, wherein R² is R²-9.536(=formula 1.2574).

Table 2575. Compounds of formula I.C, wherein R² is R²-9.537(=formula 1.2575).

Table 2576. Compounds of formula I.C, wherein R² is R²-9.538(=formula 1.2576).

Table 2577. Compounds of formula I.C, wherein R² is R²-9.539(=formula 1.2577).

Table 2578. Compounds of formula I.C, wherein R² is R²-9.540(=formula 1.2578).

Table 2579. Compounds of formula I.C, wherein R² is R²-9.541(=formula 1.2579).

Table 2580. Compounds of formula I.C, wherein R² is R²-9.542(=formula 1.2580).

Table 2581. Compounds of formula I.C, wherein R² is R²-9.543(=formula 1.2581).

Table 2582. Compounds of formula I.C, wherein R² is R²-9.544(=formula 1.2582).

Table 2583. Compounds of formula I.C, wherein R² is R²-9.545(=formula 1.2583).

Table 2584. Compounds of formula I.C, wherein R² is R²-9.546(=formula 1.2584).

Table 2585. Compounds of formula I.C, wherein R² is R²-9.547(=formula 1.2585).

Table 2586. Compounds of formula I.C, wherein R² is R²-9.548(=formula 1.2586).

Table 2587. Compounds of formula I.C, wherein R² is R²-9.549(=formula 1.2587).

Table 2588. Compounds of formula I.C, wherein R² is R²-9.550(=formula 1.2588).

Table 2589. Compounds of formula I.C, wherein R² is R²-9.551(=formula 1.2589).

Table 2590. Compounds of formula I.C, wherein R² is R²-9.552(=formula 1.2590).

Table 2591. Compounds of formula I.C, wherein R² is R²-9.553(=formula 1.2591).

Table 2592. Compounds of formula I.C, wherein R² is R²-9.554(=formula 1.2592).

Table 2593. Compounds of formula I.C, wherein R² is R²-9.555(=formula 1.2593).

Table 2594. Compounds of formula I.C, wherein R² is R²-9.556(=formula 1.2594).

Table 2595. Compounds of formula I.C, wherein R² is R²-9.557(=formula 1.2595).

Table 2596. Compounds of formula I.C, wherein R² is R²-9.558(=formula 1.2596).

Table 2597. Compounds of formula I.C, wherein R² is R²-9.559(=formula 1.2597).

Table 2598. Compounds of formula I.C, wherein R² is R²-9.560 (=formula 1.2598).

Table 2599. Compounds of formula I.C, wherein R² is R²-9.561(=formula 1.2599).

Table 2600. Compounds of formula I.C, wherein R² is R²-9.562(=formula 1.2600).

Table 2601. Compounds of formula I.C, wherein R² is R²-9.563(=formula 1.2601).

Table 2602. Compounds of formula I.C, wherein R² is R²-9.564(=formula 1.2602).

Table 2603. Compounds of formula I.C, wherein R² is R²-9.565(=formula 1.2603).

Table 2604. Compounds of formula I.C, wherein R² is R²-9.566(=formula 1.2604).

Table 2605. Compounds of formula I.C, wherein R² is R²-9.567(=formula 1.2605).

Table 2606. Compounds of formula I.C, wherein R² is R²-9.568(=formula 1.2606).

Table 2607. Compounds of formula I.C, wherein R² is R²-9.569(=formula 1.2607).

Table 2608. Compounds of formula I.C, wherein R² is R²-9.570(=formula 1.2608).

Table 2609. Compounds of formula I.C, wherein R² is R²-9.571(=formula 1.2609).

Table 2610. Compounds of formula I.C, wherein R² is R²-9.572(=formula 1.2610).

Table 2611. Compounds of formula I.C, wherein R² is R²-9.573(=formula 1.2611).

Table 2612. Compounds of formula I.C, wherein R² is R²-9.574(=formula 1.2612).

Table 2613. Compounds of formula I.C, wherein R² is R²-9.575(=formula 1.2613).

Table 2614. Compounds of formula I.C, wherein R² is R²-9.576(=formula 1.2614).

Table 2615. Compounds of formula I.C, wherein R² is R²-9.577(=formula 1.2615).

Table 2616. Compounds of formula I.C, wherein R² is R²-9.578(=formula 1.2616).

Table 2617. Compounds of formula I.C, wherein R² is R²-9.579(=formula 1.2617).

Table 2618. Compounds of formula I.C, wherein R² is R²-9.580(=formula 1.2618).

Table 2619. Compounds of formula I.C, wherein R² is R²-9.581(=formula 1.2619).

Table 2620. Compounds of formula I.C, wherein R² is R²-9.582(=formula 1.2620).

Table 2621. Compounds of formula I.C, wherein R² is R²-9.583(=formula 1.2621).

Table 2622. Compounds of formula I.C, wherein R² is R²-9.584(=formula 1.2622).

Table 2623. Compounds of formula I.C, wherein R² is R²-9.585(=formula 1.2623).

Table 2624. Compounds of formula I.C, wherein R² is R²-9.586(=formula 1.2624).

Table 2625. Compounds of formula I.C, wherein R² is R²-9.587(=formula 1.2625).

Table 2626. Compounds of formula I.C, wherein R² is R²-9.588(=formula 1.2626).

Table 2627. Compounds of formula I.C, wherein R² is R²-9.589(=formula 1.2627).

Table 2628. Compounds of formula I.C, wherein R² is R²-9.590(=formula 1.2628).

Table 2629. Compounds of formula I.C, wherein R² is R²-9.591(=formula 1.2629).

Table 2630. Compounds of formula I.C, wherein R² is R²-9.592(=formula 1.2630).

Table 2631. Compounds of formula I.C, wherein R² is R²-9.593(=formula 1.2631).

Table 2632. Compounds of formula I.C, wherein R² is R²-9.594(=formula 1.2632).

Table 2633. Compounds of formula I.C, wherein R² is R²-9.595(=formula 1.2633).

Table 2634. Compounds of formula I.C, wherein R² is R²-9.596(=formula 1.2634).

Table 2635. Compounds of formula I.C, wherein R² is R²-9.597(=formula 1.2635).

Table 2636. Compounds of formula I.C, wherein R² is R²-9.598(=formula 1.2636).

Table 2637. Compounds of formula I.C, wherein R² is R²-9.599(=formula 1.2637).

Table 2638. Compounds of formula I.C, wherein R² is R²-9.600(=formula 1.2638).

Table 2639. Compounds of formula I.C, wherein R² is R²-9.601(=formula 1.2639).

Table 2640. Compounds of formula I.C, wherein R² is R²-9.602(=formula 1.2640).

Table 2641. Compounds of formula I.C, wherein R² is R²-9.603(=formula 1.2641).

Table 2642. Compounds of formula I.C, wherein R² is R²-9.604(=formula 1.2642).

Table 2643. Compounds of formula I.C, wherein R² is R²-9.605(=formula 1.2643).

Table 2644. Compounds of formula I.C, wherein R² is R²-9.606(=formula 1.2644).

Table 2645. Compounds of formula I.C, wherein R² is R²-9.607(=formula 1.2645).

Table 2646. Compounds of formula I.C, wherein R² is R²-9.608(=formula 1.2646).

Table 2647. Compounds of formula I.C, wherein R² is R²-9.609(=formula 1.2647).

Table 2648. Compounds of formula I.C, wherein R² is R²-9.610(=formula 1.2648).

Table 2649. Compounds of formula I.C, wherein R² is R²-9.611(=formula 1.2649).

Table 2650. Compounds of formula I.C, wherein R² is R²-9.612(=formula 1.2650).

Table 2651. Compounds of formula I.C, wherein R² is R²-9.613(=formula 1.2651).

Table 2652. Compounds of formula I.C, wherein R² is R²-9.614(=formula 1.2652).

Table 2653. Compounds of formula I.C, wherein R² is R²-9.615(=formula 1.2653).

Table 2654. Compounds of formula I.C, wherein R² is R²-9.616(=formula 1.2654).

Table 2655. Compounds of formula I.C, wherein R² is R²-9.617(=formula 1.2655).

Table 2656. Compounds of formula I.C, wherein R² is R²-9.618(=formula 1.2656).

Table 2657. Compounds of formula I.C, wherein R² is R²-9.619(=formula 1.2657).

Table 2658. Compounds of formula I.C, wherein R² is R²-9.620(=formula 1.2658).

Table 2659. Compounds of formula I.C, wherein R² is R²-9.621(=formula 1.2659).

Table 2660. Compounds of formula I.C, wherein R² is R²-9.622(=formula 1.2660).

Table 2661. Compounds of formula I.C, wherein R² is R²-9.623(=formula 1.2661).

Table 2662. Compounds of formula I.C, wherein R² is R²-9.624(=formula 1.2662).

Table 2663. Compounds of formula I.C, wherein R² is R²-9.625(=formula 1.2663).

Table 2664. Compounds of formula I.C, wherein R² is R²-9.626(=formula 1.2664).

Table 2665. Compounds of formula I.C, wherein R² is R²-9.627(=formula 1.2665).

Table 2666. Compounds of formula I.C, wherein R² is R²-9.628(=formula 1.2666).

Table 2667. Compounds of formula I.C, wherein R² is R²-9.629(=formula 1.2667).

Table 2668. Compounds of formula I.C, wherein R² is R²-9.630(=formula 1.2668).

Table 2669. Compounds of formula I.C, wherein R² is R²-9.631(=formula 1.2669).

Table 2670. Compounds of formula I.C, wherein R² is R²-9.632(=formula 1.2670).

Table 2671. Compounds of formula I.C, wherein R² is R²-9.633(=formula 1.2671).

Table 2672. Compounds of formula I.C, wherein R² is R²-9.634(=formula 1.2672).

Table 2673. Compounds of formula I.C, wherein R² is R²-9.635(=formula 1.2673).

Table 2674. Compounds of formula I.C, wherein R² is R²-9.636(=formula 1.2674).

Table 2675. Compounds of formula I.C, wherein R² is R²-9.637(=formula 1.2675).

Table 2676. Compounds of formula I.C, wherein R² is R²-9.638(=formula 1.2676).

Table 2677. Compounds of formula I.C, wherein R² is R²-9.639(=formula 1.2677).

Table 2678. Compounds of formula I.C, wherein R² is R²-9.640(=formula 1.2678).

Table 2679. Compounds of formula I.C, wherein R² is R²-9.641(=formula 1.2679).

Table 2680. Compounds of formula I.C, wherein R² is R²-9.642(=formula 1.2680).

Table 2681. Compounds of formula I.C, wherein R² is R²-9.643(=formula 1.2681).

Table 2682. Compounds of formula I.C, wherein R² is R²-9.644(=formula 1.2682).

Table 2683. Compounds of formula I.C, wherein R² is R²-9.645(=formula 1.2683).

Table 2684. Compounds of formula I.C, wherein R² is R²-9.646(=formula 1.2684).

Table 2685. Compounds of formula I.C, wherein R² is R²-9.647(=formula 1.2685).

Table 2686. Compounds of formula I.C, wherein R² is R²-9.648(=formula 1.2686).

Table 2687. Compounds of formula I.C, wherein R² is R²-9.649(=formula 1.2687).

Table 2688. Compounds of formula I.C, wherein R² is R²-9.650(=formula 1.2688).

Table 2689. Compounds of formula I.C, wherein R² is R²-9.651(=formula 1.2689).

Table 2690. Compounds of formula I.C, wherein R² is R²-9.652(=formula 1.2690).

Table 2691. Compounds of formula I.C, wherein R² is R²-9.653(=formula 1.2691).

Table 2692. Compounds of formula I.C, wherein R² is R²-9.654(=formula 1.2692).

Table 2693. Compounds of formula I.C, wherein R² is R²-9.655(=formula 1.2693).

Table 2694. Compounds of formula I.C, wherein R² is R²-9.656(=formula 1.2694).

Table 2695. Compounds of formula I.C, wherein R² is R²-9.657(=formula 1.2695).

Table 2696. Compounds of formula I.C, wherein R² is R²-9.658(=formula 1.2696).

Table 2697. Compounds of formula I.C, wherein R² is R²-9.659(=formula 1.2697).

Table 2698. Compounds of formula I.C, wherein R² is R²-9.660(=formula 1.2698).

Table 2699. Compounds of formula I.C, wherein R² is R²-9.661(=formula 1.2699).

Table 2700. Compounds of formula I.C, wherein R² is R²-9.662(=formula 1.2700).

Table 2701. Compounds of formula I.C, wherein R² is R²-9.663(=formula 1.2701).

Table 2702. Compounds of formula I.C, wherein R² is R²-9.664(=formula 1.2702).

Table 2703. Compounds of formula I.C, wherein R² is R²-9.665(=formula 1.2703).

Table 2704. Compounds of formula I.C, wherein R² is R²-9.666(=formula 1.2704).

Table 2705. Compounds of formula I.C, wherein R² is R²-9.667(=formula 1.2705).

Table 2706. Compounds of formula I.C, wherein R² is R²-9.668(=formula 1.2706).

Table 2707. Compounds of formula I.C, wherein R² is R²-9.669(=formula 1.2707).

Table 2708. Compounds of formula I.C, wherein R² is R²-9.670(=formula 1.2708).

Table 2709. Compounds of formula I.C, wherein R² is R²-9.671(=formula 1.2709).

Table 2710. Compounds of formula I.C, wherein R² is R²-9.672(=formula 1.2710).

Table 2711. Compounds of formula I.C, wherein R² is R²-9.673(=formula 1.2711).

Table 2712. Compounds of formula I.C, wherein R² is R²-9.674(=formula 1.2712).

Table 2713. Compounds of formula I.C, wherein R² is R²-9.675(=formula 1.2713).

Table 2714. Compounds of formula I.C, wherein R² is R²-9.676(=formula 1.2714).

Table 2715. Compounds of formula I.C, wherein R² is R²-10.1(=formula 1.2715).

Table 2716. Compounds of formula I.C, wherein R² is R²-10.2(=formula 1.2716).

Table 2717. Compounds of formula I.C, wherein R² is R²-10.3(=formula 1.2717).

Table 2718. Compounds of formula I.C, wherein R² is R²-10.4(=formula 1.2718).

Table 2719. Compounds of formula I.C, wherein R² is R²-10.5(=formula 1.2719).

Table 2720. Compounds of formula I.C, wherein R² is R²-10.6(=formula 1.2720).

Table 2721. Compounds of formula I.C, wherein R² is R²-10.7(=formula 1.2721).

Table 2722. Compounds of formula I.C, wherein R² is R²-10.8(=formula 1.2722).

Table 2723. Compounds of formula I.C, wherein R² is R²-10.9(=formula 1.2723).

Table 2724. Compounds of formula I.C, wherein R² is R²-10.10(=formula 1.2724).

Table 2725. Compounds of formula I.C, wherein R² is R²-10.11(=formula 1.2725).

Table 2726. Compounds of formula I.C, wherein R² is R²-10.12(=formula 1.2726).

Table 2727. Compounds of formula I.C, wherein R² is R²-10.13(=formula 1.2727).

Table 2728. Compounds of formula I.C, wherein R² is R²-10.14(=formula 1.2728).

Table 2729. Compounds of formula I.C, wherein R² is R²-10.15(=formula 1.2729).

Table 2730. Compounds of formula I.C, wherein R² is R²-10.16(=formula 1.2730).

Table 2731. Compounds of formula I.C, wherein R² is R²-10.17(=formula 1.2731).

Table 2732. Compounds of formula I.C, wherein R² is R²-10.18(=formula 1.2732).

Table 2733. Compounds of formula I.C, wherein R² is R²-10.19(=formula 1.2733).

Table 2734. Compounds of formula I.C, wherein R² is R²-10.20(=formula 1.2734).

Table 2735. Compounds of formula I.C, wherein R² is R²-10.21(=formula 1.2735).

Table 2736. Compounds of formula I.C, wherein R² is R²-10.22(=formula 1.2736).

Table 2737. Compounds of formula I.C, wherein R² is R²-10.23(=formula 1.2737).

Table 2738. Compounds of formula I.C, wherein R² is R²-10.24(=formula 1.2738).

Table 2739. Compounds of formula I.C, wherein R² is R²-10.25(=formula 1.2739).

Table 2740. Compounds of formula I.C, wherein R² is R²-10.26(=formula 1.2740).

Table 2741. Compounds of formula I.C, wherein R² is R²-10.27(=formula 1.2741).

Table 2742. Compounds of formula I.C, wherein R² is R²-10.28(=formula 1.2742).

Table 2743. Compounds of formula I.C, wherein R² is R²-10.29(=formula 1.2743).

Table 2744. Compounds of formula I.C, wherein R² is R²-10.30(=formula 1.2744).

Table 2745. Compounds of formula I.C, wherein R² is R²-10.31(=formula 1.2745).

Table 2746. Compounds of formula I.C, wherein R² is R²-10.32(=formula 1.2746).

Table 2747. Compounds of formula I.C, wherein R² is R²-10.33(=formula 1.2747).

Table 2748. Compounds of formula I.C, wherein R² is R²-10.34(=formula 1.2748).

Table 2749. Compounds of formula I.C, wherein R² is R²-10.35(=formula 1.2749).

Table 2750. Compounds of formula I.C, wherein R² is R²-10.36(=formula 1.2750).

Table 2751. Compounds of formula I.C, wherein R² is R²-10.37(=formula 1.2751).

Table 2752. Compounds of formula I.C, wherein R² is R²-10.38(=formula 1.2752).

Table 2753. Compounds of formula I.C, wherein R² is R²-10.39(=formula 1.2753).

Table 2754. Compounds of formula I.C, wherein R² is R²-10.40(=formula 1.2754).

Table 2755. Compounds of formula I.C, wherein R² is R²-10.41(=formula 1.2755).

Table 2756. Compounds of formula I.C, wherein R² is R²-10.42(=formula 1.2756).

Table 2757. Compounds of formula I.C, wherein R² is R²-10.43(=formula 1.2757).

Table 2758. Compounds of formula I.C, wherein R² is R²-10.44(=formula 1.2758).

Table 2759. Compounds of formula I.C, wherein R² is R²-10.45(=formula 1.2759).

Table 2760. Compounds of formula I.C, wherein R² is R²-10.46(=formula 1.2760).

Table 2761. Compounds of formula I.C, wherein R² is R²-10.47(=formula 1.2761).

Table 2762. Compounds of formula I.C, wherein R² is R²-10.48(=formula 1.2762).

Table 2763. Compounds of formula I.C, wherein R² is R²-10.49(=formula 1.2763).

Table 2764. Compounds of formula I.C, wherein R² is R²-10.50(=formula 1.2764).

Table 2765. Compounds of formula I.C, wherein R² is R²-10.51(=formula 1.2765).

Table 2766. Compounds of formula I.C, wherein R² is R²-10.52(=formula 1.2766).

Table 2767. Compounds of formula I.C, wherein R² is R²-10.53(=formula 1.2767).

Table 2768. Compounds of formula I.C, wherein R² is R²-10.54(=formula 1.2768).

Table 2769. Compounds of formula I.C, wherein R² is R²-10.55(=formula 1.2769).

Table 2770. Compounds of formula I.C, wherein R² is R²-10.56(=formula 1.2770).

Table 2771. Compounds of formula I.C, wherein R² is R²-10.57(=formula 1.2771).

Table 2772. Compounds of formula I.C, wherein R² is R²-10.58(=formula 1.2772).

Table 2773. Compounds of formula I.C, wherein R² is R²-10.59(=formula 1.2773).

Table 2774. Compounds of formula I.C, wherein R² is R²-10.60(=formula 1.2774).

Table 2775. Compounds of formula I.C, wherein R² is R²-10.61(=formula 1.2775).

Table 2776. Compounds of formula I.C, wherein R² is R²-10.62(=formula 1.2776).

Table 2777. Compounds of formula I.C, wherein R² is R²-10.63(=formula 1.2777).

Table 2778. Compounds of formula I.C, wherein R² is R²-10.64(=formula 1.2778).

Table 2779. Compounds of formula I.C, wherein R² is R²-10.65(=formula 1.2779).

Table 2780. Compounds of formula I.C, wherein R² is R²-10.66(=formula 1.2780).

Table 2781. Compounds of formula I.C, wherein R² is R²-10.67(=formula 1.2781).

Table 2782. Compounds of formula I.C, wherein R² is R²-10.68(=formula 1.2782).

Table 2783. Compounds of formula I.C, wherein R² is R²-10.69(=formula 1.2783).

Table 2784. Compounds of formula I.C, wherein R² is R²-10.70(=formula 1.2784).

Table 2785. Compounds of formula I.C, wherein R² is R²-10.71(=formula 1.2785).

Table 2786. Compounds of formula I.C, wherein R² is R²-10.72(=formula 1.2786).

Table 2787. Compounds of formula I.C, wherein R² is R²-10.73(=formula 1.2787).

Table 2788. Compounds of formula I.C, wherein R² is R²-10.74(=formula 1.2788).

Table 2789. Compounds of formula I.C, wherein R² is R²-10.75(=formula 1.2789).

Table 2790. Compounds of formula I.C, wherein R² is R²-10.76(=formula 1.2790).

Table 2791. Compounds of formula I.C, wherein R² is R²-10.77(=formula 1.2791).

Table 2792. Compounds of formula I.C, wherein R² is R²-10.78(=formula 1.2792).

Table 2793. Compounds of formula I.C, wherein R² is R²-10.79(=formula 1.2793).

Table 2794. Compounds of formula I.C, wherein R² is R²-10.80(=formula 1.2794).

Table 2795. Compounds of formula I.C, wherein R² is R²-10.81(=formula 1.2795).

Table 2796. Compounds of formula I.C, wherein R² is R²-10.82(=formula 1.2796).

Table 2797. Compounds of formula I.C, wherein R² is R²-10.83(=formula 1.2797).

Table 2798. Compounds of formula I.C, wherein R² is R²-10.84(=formula 1.2798).

Table 2799. Compounds of formula I.C, wherein R² is R²-10.85(=formula 1.2799).

Table 2800. Compounds of formula I.C, wherein R² is R²-10.86(=formula 1.2800).

Table 2801. Compounds of formula I.C, wherein R² is R²-10.87(=formula 1.2801).

Table 2802. Compounds of formula I.C, wherein R² is R²-10.88(=formula 1.2802).

Table 2803. Compounds of formula I.C, wherein R² is R²-10.89(=formula 1.2803).

Table 2804. Compounds of formula I.C, wherein R² is R²-10.90(=formula 1.2804).

Table 2805. Compounds of formula I.C, wherein R² is R²-10.91(=formula 1.2805).

Table 2806. Compounds of formula I.C, wherein R² is R²-10.92(=formula 1.2806).

Table 2807. Compounds of formula I.C, wherein R² is R²-10.93(=formula 1.2807).

Table 2808. Compounds of formula I.C, wherein R² is R²-10.94(=formula 1.2808).

Table 2809. Compounds of formula I.C, wherein R² is R²-10.95(=formula 1.2809).

Table 2810. Compounds of formula I.C, wherein R² is R²-10.96(=formula 1.2810).

Table 2811. Compounds of formula I.C, wherein R² is R²-10.97(=formula 1.2811).

Table 2812. Compounds of formula I.C, wherein R² is R²-10.98(=formula 1.2812).

Table 2813. Compounds of formula I.C, wherein R² is R²-10.99(=formula 1.2813).

Table 2814. Compounds of formula I.C, wherein R² is R²-10.100(=formula 1.2814).

Table 2815. Compounds of formula I.C, wherein R² is R²-10.101(=formula 1.2815).

Table 2816. Compounds of formula I.C, wherein R² is R²-10.102(=formula 1.2816).

Table 2817. Compounds of formula I.C, wherein R² is R²-10.103(=formula 1.2817).

Table 2818. Compounds of formula I.C, wherein R² is R²-10.104(=formula 1.2818).

Table 2819. Compounds of formula I.C, wherein R² is R²-10.105(=formula 1.2819).

Table 2820. Compounds of formula I.C, wherein R² is R²-10.106(=formula 1.2820).

Table 2821. Compounds of formula I.C, wherein R² is R²-10.107(=formula 1.2821).

Table 2822. Compounds of formula I.C, wherein R² is R²-10.108(=formula 1.2822).

Table 2823. Compounds of formula I.C, wherein R² is R²-10.109(=formula 1.2823).

Table 2824. Compounds of formula I.C, wherein R² is R²-10.110(=formula 1.2824).

Table 2825. Compounds of formula I.C, wherein R² is R²-10.111(=formula 1.2825).

Table 2826. Compounds of formula I.C, wherein R² is R²-10.112(=formula 1.2826).

Table 2827. Compounds of formula I.C, wherein R² is R²-10.113(=formula 1.2827).

Table 2828. Compounds of formula I.C, wherein R² is R²-10.114(=formula 1.2828).

Table 2829. Compounds of formula I.C, wherein R² is R²-10.115(=formula 1.2829).

Table 2830. Compounds of formula I.C, wherein R² is R²-10.116(=formula 1.2830).

Table 2831. Compounds of formula I.C, wherein R² is R²-10.117(=formula 1.2831).

Table 2832. Compounds of formula I.C, wherein R² is R²-10.118(=formula 1.2832).

Table 2833. Compounds of formula I.C, wherein R² is R²-10.119(=formula 1.2833).

Table 2834. Compounds of formula I.C, wherein R² is R²-10.120(=formula 1.2834).

Table 2835. Compounds of formula I.C, wherein R² is R²-10.121(=formula 1.2835).

Table 2836. Compounds of formula I.C, wherein R² is R²-10.122(=formula 1.2836).

Table 2837. Compounds of formula I.C, wherein R² is R²-10.123(=formula 1.2837).

Table 2838. Compounds of formula I.C, wherein R² is R²-10.124(=formula 1.2838).

Table 2839. Compounds of formula I.C, wherein R² is R²-10.125(=formula 1.2839).

Table 2840. Compounds of formula I.C, wherein R² is R²-10.126(=formula 1.2840).

Table 2841. Compounds of formula I.C, wherein R² is R²-10.127(=formula 1.2841).

Table 2842. Compounds of formula I.C, wherein R² is R²-10.128(=formula 1.2842).

Table 2843. Compounds of formula I.C, wherein R² is R²-10.129(=formula 1.2843).

Table 2844. Compounds of formula I.C, wherein R² is R²-10.130(=formula 1.2844).

Table 2845. Compounds of formula I.C, wherein R² is R²-10.131(=formula 1.2845).

Table 2846. Compounds of formula I.C, wherein R² is R²-10.132(=formula 1.2846).

Table 2847. Compounds of formula I.C, wherein R² is R²-10.133(=formula 1.2847).

Table 2848. Compounds of formula I.C, wherein R² is R²-10.134(=formula 1.2848).

Table 2849. Compounds of formula I.C, wherein R² is R²-10.135(=formula 1.2849).

Table 2850. Compounds of formula I.C, wherein R² is R²-10.136(=formula 1.2850).

Table 2851. Compounds of formula I.C, wherein R² is R²-10.137(=formula 1.2851).

Table 2852. Compounds of formula I.C, wherein R² is R²-10.138(=formula 1.2852).

Table 2853. Compounds of formula I.C, wherein R² is R²-10.139(=formula 1.2853).

Table 2854. Compounds of formula I.C, wherein R² is R²-10.140(=formula 1.2854).

Table 2855. Compounds of formula I.C, wherein R² is R²-10.141(=formula 1.2855).

Table 2856. Compounds of formula I.C, wherein R² is R²-10.142(=formula 1.2856).

Table 2857. Compounds of formula I.C, wherein R² is R²-10.143(=formula 1.2857).

Table 2858. Compounds of formula I.C, wherein R² is R²-10.144(=formula 1.2858).

Table 2859. Compounds of formula I.C, wherein R² is R²-10.145(=formula 1.2859).

Table 2860. Compounds of formula I.C, wherein R² is R²-10.146(=formula 1.2860).

Table 2861. Compounds of formula I.C, wherein R² is R²-10.147(=formula 1.2861).

Table 2862. Compounds of formula I.C, wherein R² is R²-10.148(=formula 1.2862).

Table 2863. Compounds of formula I.C, wherein R² is R²-10.149(=formula 1.2863).

Table 2864. Compounds of formula I.C, wherein R² is R²-10.150(=formula 1.2864).

Table 2865. Compounds of formula I.C, wherein R² is R²-10.151(=formula 1.2865).

Table 2866. Compounds of formula I.C, wherein R² is R²-10.152(=formula 1.2866).

Table 2867. Compounds of formula I.C, wherein R² is R²-10.153(=formula 1.2867).

Table 2868. Compounds of formula I.C, wherein R² is R²-10.154(=formula 1.2868).

Table 2869. Compounds of formula I.C, wherein R² is R²-10.155(=formula 1.2869).

Table 2870. Compounds of formula I.C, wherein R² is R²-10.156(=formula 1.2870).

Table 2871. Compounds of formula I.C, wherein R² is R²-10.157(=formula 1.2871).

Table 2872. Compounds of formula I.C, wherein R² is R²-10.158(=formula 1.2872).

Table 2873. Compounds of formula I.C, wherein R² is R²-10.159(=formula 1.2873).

Table 2874. Compounds of formula I.C, wherein R² is R²-10.160(=formula 1.2874).

Table 2875. Compounds of formula I.C, wherein R² is R²-10.161(=formula 1.2875).

Table 2876. Compounds of formula I.C, wherein R² is R²-10.162(=formula 1.2876).

Table 2877. Compounds of formula I.C, wherein R² is R²-10.163(=formula 1.2877).

Table 2878. Compounds of formula I.C, wherein R² is R²-10.164(=formula 1.2878).

Table 2879. Compounds of formula I.C, wherein R² is R²-10.165(=formula 1.2879).

Table 2880. Compounds of formula I.C, wherein R² is R²-10.166(=formula 1.2880).

Table 2881. Compounds of formula I.C, wherein R² is R²-10.167(=formula 1.2881).

Table 2882. Compounds of formula I.C, wherein R² is R²-10.168(=formula 1.2882).

Table 2883. Compounds of formula I.C, wherein R² is R²-10.169(=formula 1.2883).

Table 2884. Compounds of formula I.C, wherein R² is R²-10.170(=formula 1.2884).

Table 2885. Compounds of formula I.C, wherein R² is R²-10.171(=formula 1.2885).

Table 2886. Compounds of formula I.C, wherein R² is R²-10.172(=formula 1.2886).

Table 2887. Compounds of formula I.C, wherein R² is R²-10.173(=formula 1.2887).

Table 2888. Compounds of formula I.C, wherein R² is R²-10.174(=formula 1.2888).

Table 2889. Compounds of formula I.C, wherein R² is R²-10.175(=formula 1.2889).

Table 2890. Compounds of formula I.C, wherein R² is R²-10.176(=formula 1.2890).

Table 2891. Compounds of formula I.C, wherein R² is R²-10.177(=formula 1.2891).

Table 2892. Compounds of formula I.C, wherein R² is R²-10.178(=formula 1.2892).

Table 2893. Compounds of formula I.C, wherein R² is R²-10.179(=formula 1.2893).

Table 2894. Compounds of formula I.C, wherein R² is R²-10.180(=formula 1.2894).

Table 2895. Compounds of formula I.C, wherein R² is R²-10.181(=formula 1.2895).

Table 2896. Compounds of formula I.C, wherein R² is R²-10.182(=formula 1.2896).

Table 2897. Compounds of formula I.C, wherein R² is R²-10.183(=formula 1.2897).

Table 2898. Compounds of formula I.C, wherein R² is R²-10.184(=formula 1.2898).

Table 2899. Compounds of formula I.C, wherein R² is R²-10.185(=formula 1.2899).

Table 2900. Compounds of formula I.C, wherein R² is R²-10.186(=formula 1.2900).

Table 2901. Compounds of formula I.C, wherein R² is R²-10.187(=formula 1.2901).

Table 2902. Compounds of formula I.C, wherein R² is R²-10.188(=formula 1.2902).

Table 2903. Compounds of formula I.C, wherein R² is R²-10.189(=formula 1.2903).

Table 2904. Compounds of formula I.C, wherein R² is R²-10.190(=formula 1.2904).

Table 2905. Compounds of formula I.C, wherein R² is R²-10.191(=formula 1.2905).

Table 2906. Compounds of formula I.C, wherein R² is R²-10.192(=formula 1.2906).

Table 2907. Compounds of formula I.C, wherein R² is R²-10.193(=formula 1.2907).

Table 2908. Compounds of formula I.C, wherein R² is R²-10.194(=formula 1.2908).

Table 2909. Compounds of formula I.C, wherein R² is R²-10.195(=formula 1.2909).

Table 2910. Compounds of formula I.C, wherein R² is R²-10.196(=formula 1.2910).

Table 2911. Compounds of formula I.C, wherein R² is R²-10.197(=formula 1.2911).

Table 2912. Compounds of formula I.C, wherein R² is R²-10.198(=formula 1.2912).

Table 2913. Compounds of formula I.C, wherein R² is R²-10.199(=formula 1.2913).

Table 2914. Compounds of formula I.C, wherein R² is R²-10.200(=formula 1.2914).

Table 2915. Compounds of formula I.C, wherein R² is R²-10.201(=formula 1.2915).

Table 2916. Compounds of formula I.C, wherein R² is R²-10.202(=formula 1.2916).

Table 2917. Compounds of formula I.C, wherein R² is R²-10.203(=formula 1.2917).

Table 2918. Compounds of formula I.C, wherein R² is R²-10.204(=formula 1.2918).

Table 2919. Compounds of formula I.C, wherein R² is R²-10.205(=formula 1.2919).

Table 2920. Compounds of formula I.C, wherein R² is R²-10.206(=formula 1.2920).

Table 2921. Compounds of formula I.C, wherein R² is R²-10.207(=formula 1.2921).

Table 2922. Compounds of formula I.C, wherein R² is R²-10.208(=formula 1.2922).

Table 2923. Compounds of formula I.C, wherein R² is R²-10.209(=formula 1.2923).

Table 2924. Compounds of formula I.C, wherein R² is R²-10.210(=formula 1.2924).

Table 2925. Compounds of formula I.C, wherein R² is R²-10.211(=formula 1.2925).

Table 2926. Compounds of formula I.C, wherein R² is R²-10.212(=formula 1.2926).

Table 2927. Compounds of formula I.C, wherein R² is R²-10.213(=formula 1.2927).

Table 2928. Compounds of formula I.C, wherein R² is R²-10.214(=formula 1.2928).

Table 2929. Compounds of formula I.C, wherein R² is R²-10.215(=formula 1.2929).

Table 2930. Compounds of formula I.C, wherein R² is R²-10.216(=formula 1.2930).

Table 2931. Compounds of formula I.C, wherein R² is R²-10.217(=formula 1.2931).

Table 2932. Compounds of formula I.C, wherein R² is R²-10.218(=formula 1.2932).

Table 2933. Compounds of formula I.C, wherein R² is R²-10.219(=formula 1.2933).

Table 2934. Compounds of formula I.C, wherein R² is R²-10.220(=formula 1.2934).

Table 2935. Compounds of formula I.C, wherein R² is R²-10.221(=formula 1.2935).

Table 2936. Compounds of formula I.C, wherein R² is R²-10.222(=formula 1.2936).

Table 2937. Compounds of formula I.C, wherein R² is R²-10.223(=formula 1.2937).

Table 2938. Compounds of formula I.C, wherein R² is R²-10.224(=formula 1.2938).

Table 2939. Compounds of formula I.C, wherein R² is R²-10.225(=formula 1.2939).

Table 2940. Compounds of formula I.C, wherein R² is R²-10.226(=formula 1.2940).

Table 2941. Compounds of formula I.C, wherein R² is R²-10.227(=formula 1.2941).

Table 2942. Compounds of formula I.C, wherein R² is R²-10.228(=formula 1.2942).

Table 2943. Compounds of formula I.C, wherein R² is R²-10.229(=formula 1.2943).

Table 2944. Compounds of formula I.C, wherein R² is R²-10.230(=formula 1.2944).

Table 2945. Compounds of formula I.C, wherein R² is R²-10.231(=formula 1.2945).

Table 2946. Compounds of formula I.C, wherein R² is R²-10.232(=formula 1.2946).

Table 2947. Compounds of formula I.C, wherein R² is R²-10.233(=formula 1.2947).

Table 2948. Compounds of formula I.C, wherein R² is R²-10.234(=formula 1.2948).

Table 2949. Compounds of formula I.C, wherein R² is R²-10.235(=formula 1.2949).

Table 2950. Compounds of formula I.C, wherein R² is R²-10.236(=formula 1.2950).

Table 2951. Compounds of formula I.C, wherein R² is R²-10.237(=formula 1.2951).

Table 2952. Compounds of formula I.C, wherein R² is R²-10.238(=formula 1.2952).

Table 2953. Compounds of formula I.C, wherein R² is R²-10.239(=formula 1.2953).

Table 2954. Compounds of formula I.C, wherein R² is R²-10.240(=formula 1.2954).

Table 2955. Compounds of formula I.C, wherein R² is R²-10.241(=formula 1.2955).

Table 2956. Compounds of formula I.C, wherein R² is R²-10.242(=formula 1.2956).

Table 2957. Compounds of formula I.C, wherein R² is R²-10.243(=formula 1.2957).

Table 2958. Compounds of formula I.C, wherein R² is R²-10.244(=formula 1.2958).

Table 2959. Compounds of formula I.C, wherein R² is R²-10.245(=formula 1.2959).

Table 2960. Compounds of formula I.C, wherein R² is R²-10.246(=formula 1.2960).

Table 2961. Compounds of formula I.C, wherein R² is R²-10.247(=formula 1.2961).

Table 2962. Compounds of formula I.C, wherein R² is R²-10.248(=formula 1.2962).

Table 2963. Compounds of formula I.C, wherein R² is R²-10.249(=formula 1.2963).

Table 2964. Compounds of formula I.C, wherein R² is R²-10.250(=formula 1.2964).

Table 2965. Compounds of formula I.C, wherein R² is R²-10.251(=formula 1.2965).

Table 2966. Compounds of formula I.C, wherein R² is R²-10.252(=formula 1.2966).

Table 2967. Compounds of formula I.C, wherein R² is R²-10.253(=formula 1.2967).

Table 2968. Compounds of formula I.C, wherein R² is R²-10.254(=formula 1.2968).

Table 2969. Compounds of formula I.C, wherein R² is R²-10.255(=formula 1.2969).

Table 2970. Compounds of formula I.C, wherein R² is R²-10.256(=formula 1.2970).

Table 2971. Compounds of formula I.C, wherein R² is R²-10.257(=formula 1.2971).

Table 2972. Compounds of formula I.C, wherein R² is R²-10.258(=formula 1.2972).

Table 2973. Compounds of formula I.C, wherein R² is R²-10.259(=formula 1.2973).

Table 2974. Compounds of formula I.C, wherein R² is R²-10.260(=formula 1.2974).

Table 2975. Compounds of formula I.C, wherein R² is R²-10.261(=formula 1.2975).

Table 2976. Compounds of formula I.C, wherein R² is R²-10.262(=formula 1.2976).

Table 2977. Compounds of formula I.C, wherein R² is R²-10.263(=formula 1.2977).

Table 2978. Compounds of formula I.C, wherein R² is R²-10.264(=formula 1.2978).

Table 2979. Compounds of formula I.C, wherein R² is R²-10.265(=formula 1.2979).

Table 2980. Compounds of formula I.C, wherein R² is R²-10.266(=formula 1.2980).

Table 2981. Compounds of formula I.C, wherein R² is R²-10.267(=formula 1.2981).

Table 2982. Compounds of formula I.C, wherein R² is R²-10.268(=formula 1.2982).

Table 2983. Compounds of formula I.C, wherein R² is R²-10.269(=formula 1.2983).

Table 2984. Compounds of formula I.C, wherein R² is R²-10.270(=formula 1.2984).

Table 2985. Compounds of formula I.C, wherein R² is R²-10.271(=formula 1.2985).

Table 2986. Compounds of formula I.C, wherein R² is R²-10.272(=formula 1.2986).

Table 2987. Compounds of formula I.C, wherein R² is R²-10.273(=formula 1.2987).

Table 2988. Compounds of formula I.C, wherein R² is R²-10.274(=formula 1.2988).

Table 2989. Compounds of formula I.C, wherein R² is R²-10.275(=formula 1.2989).

Table 2990. Compounds of formula I.C, wherein R² is R²-10.276(=formula 1.2990).

Table 2991. Compounds of formula I.C, wherein R² is R²-10.277(=formula 1.2991).

Table 2992. Compounds of formula I.C, wherein R² is R²-10.278(=formula 1.2992).

Table 2993. Compounds of formula I.C, wherein R² is R²-10.279(=formula 1.2993.

Table 2994. Compounds of formula I.C, wherein R² is R²-10.280(=formula 1.2994).

Table 2995. Compounds of formula I.C, wherein R² is R²-10.281(=formula 1.2995).

Table 2996. Compounds of formula I.C, wherein R² is R²-10.282(=formula 1.2996).

Table 2997. Compounds of formula I.C, wherein R² is R²-10.283(=formula 1.2997).

Table 2998. Compounds of formula I.C, wherein R² is R²-10.284(=formula 1.2998).

Table 2999. Compounds of formula I.C, wherein R² is R²-10.285(=formula 1.2999).

Table 3000. Compounds of formula I.C, wherein R² is R²-10.286(=formula 1.3000).

Table 3001. Compounds of formula I.C, wherein R² is R²-10.287(=formula 1.3001).

Table 3002. Compounds of formula I.C, wherein R² is R²-10.288(=formula 1.3002).

Table 3003. Compounds of formula I.C, wherein R² is R²-10.289(=formula 1.3003).

Table 3004. Compounds of formula I.C, wherein R² is R²-10.290(=formula 1.3004).

Table 3005. Compounds of formula I.C, wherein R² is R²-10.291(=formula 1.3005).

Table 3006. Compounds of formula I.C, wherein R² is R²-10.292(=formula 1.3006).

Table 3007. Compounds of formula I.C, wherein R² is R²-10.293(=formula 1.3007).

Table 3008. Compounds of formula I.C, wherein R² is R²-10.294(=formula 1.3008).

Table 3009. Compounds of formula I.C, wherein R² is R²-10.295(=formula 1.3009).

Table 3010. Compounds of formula I.C, wherein R² is R²-10.296(=formula 1.3010).

Table 3011. Compounds of formula I.C, wherein R² is R²-10.297(=formula 1.3011).

Table 3012. Compounds of formula I.C, wherein R² is R²-10.298(=formula 1.3012).

Table 3013. Compounds of formula I.C, wherein R² is R²-10.299(=formula 1.3013).

Table 3014. Compounds of formula I.C, wherein R² is R²-10.300(=formula 1.3014).

Table 3015. Compounds of formula I.C, wherein R² is R²-10.301(=formula 1.3015).

Table 3016. Compounds of formula I.C, wherein R² is R²-10.302(=formula 1.3016).

Table 3017. Compounds of formula I.C, wherein R² is R²-10.303(=formula 1.3017).

Table 3018. Compounds of formula I.C, wherein R² is R²-10.304(=formula 1.3018).

Table 3019. Compounds of formula I.C, wherein R² is R²-10.305(=formula 1.3019).

Table 3020. Compounds of formula I.C, wherein R² is R²-10.306(=formula 1.3020).

Table 3021. Compounds of formula I.C, wherein R² is R²-10.307(=formula 1.3021).

Table 3022. Compounds of formula I.C, wherein R² is R²-10.308(=formula 1.3022).

Table 3023. Compounds of formula I.C, wherein R² is R²-10.309(=formula 1.3023).

Table 3024. Compounds of formula I.C, wherein R² is R²-10.310(=formula 1.3024).

Table 3025. Compounds of formula I.C, wherein R² is R²-10.311(=formula 1.3025).

Table 3026. Compounds of formula I.C, wherein R² is R²-10.312(=formula 1.3026).

Table 3027. Compounds of formula I.C, wherein R² is R²-10.313(=formula 1.3027).

Table 3028. Compounds of formula I.C, wherein R² is R²-10.314(=formula 1.3028).

Table 3029. Compounds of formula I.C, wherein R² is R²-10.315(=formula 1.3029).

Table 3030. Compounds of formula I.C, wherein R² is R²-10.316(=formula 1.3030).

Table 3031. Compounds of formula I.C, wherein R² is R²-10.317(=formula 1.3031).

Table 3032. Compounds of formula I.C, wherein R² is R²-10.318(=formula 1.3032).

Table 3033. Compounds of formula I.C, wherein R² is R²-10.319(=formula 1.3033).

Table 3034. Compounds of formula I.C, wherein R² is R²-10.320(=formula 1.3034).

Table 3035. Compounds of formula I.C, wherein R² is R²-10.321(=formula 1.3035).

Table 3036. Compounds of formula I.C, wherein R² is R²-10.322(=formula 1.3036).

Table 3037. Compounds of formula I.C, wherein R² is R²-10.323(=formula 1.3037).

Table 3038. Compounds of formula I.C, wherein R² is R²-10.324(=formula 1.3038).

Table 3039. Compounds of formula I.C, wherein R² is R²-10.325(=formula 1.3039).

Table 3040. Compounds of formula I.C, wherein R² is R²-10.326(=formula 1.3040).

Table 3041. Compounds of formula I.C, wherein R² is R²-10.327(=formula 1.3041).

Table 3042. Compounds of formula I.C, wherein R² is R²-10.328(=formula 1.3042).

Table 3043. Compounds of formula I.C, wherein R² is R²-10.329(=formula 1.3043).

Table 3044. Compounds of formula I.C, wherein R² is R²-10.330(=formula 1.3044).

Table 3045. Compounds of formula I.C, wherein R² is R²-10.331(=formula 1.3045).

Table 3046. Compounds of formula I.C, wherein R² is R²-10.332(=formula 1.3046).

Table 3047. Compounds of formula I.C, wherein R² is R²-10.333(=formula 1.3047).

Table 3048. Compounds of formula I.C, wherein R² is R²-10.334(=formula 1.3048).

Table 3049. Compounds of formula I.C, wherein R² is R²-10.335(=formula 1.3049).

Table 3050. Compounds of formula I.C, wherein R² is R²-10.336(=formula 1.3050).

Table 3051. Compounds of formula I.C, wherein R² is R²-10.334(=formula 1.3051).

Table 3052. Compounds of formula I.C, wherein R² is R²-10.338(=formula 1.3052).

Table 3053. Compounds of formula I.C, wherein R² is R²-10.339(=formula 1.3053).

Table 3054. Compounds of formula I.C, wherein R² is R²-10.340(=formula 1.3054).

Table 3055. Compounds of formula I.C, wherein R² is R²-10.341(=formula 1.3055).

Table 3056. Compounds of formula I.C, wherein R² is R²-10.342(=formula 1.3056).

Table 3057. Compounds of formula I.C, wherein R² is R²-10.343(=formula 1.3057).

Table 3058. Compounds of formula I.C, wherein R² is R²-10.344(=formula 1.3058).

Table 3059. Compounds of formula I.C, wherein R² is R²-10.345(=formula 1.3059).

Table 3060. Compounds of formula I.C, wherein R² is R²-10.346(=formula 1.3060).

Table 3061. Compounds of formula I.C, wherein R² is R²-10.347(=formula 1.3061).

Table 3062. Compounds of formula I.C, wherein R² is R²-10.348(=formula 1.3062).

Table 3063. Compounds of formula I.C, wherein R² is R²-10.349(=formula 1.3063).

Table 3064. Compounds of formula I.C, wherein R² is R²-10.350(=formula 1.3064).

Table 3065. Compounds of formula I.C, wherein R² is R²-10.351(=formula 1.3065).

Table 3066. Compounds of formula I.C, wherein R² is R²-10.352(=formula 1.3066).

Table 3067. Compounds of formula I.C, wherein R² is R²-10.353(=formula 1.3067).

Table 3068. Compounds of formula I.C, wherein R² is R²-10.354(=formula 1.3068).

Table 3069. Compounds of formula I.C, wherein R² is R²-10.355(=formula 1.3069).

Table 3070. Compounds of formula I.C, wherein R² is R²-10.356(=formula 1.3070).

Table 3071. Compounds of formula I.C, wherein R² is R²-10.357(=formula 1.3071).

Table 3072. Compounds of formula I.C, wherein R² is R²-10.358(=formula 1.3072).

Table 3073. Compounds of formula I.C, wherein R² is R²-10.359(=formula 1.3073).

Table 3074. Compounds of formula I.C, wherein R² is R²-10.360(=formula 1.3074).

Table 3075. Compounds of formula I.C, wherein R² is R²-10.361(=formula 1.3075).

Table 3076. Compounds of formula I.C, wherein R² is R²-10.362(=formula 1.3076).

Table 3077. Compounds of formula I.C, wherein R² is R²-10.363(=formula 1.3077).

Table 3078. Compounds of formula I.C, wherein R² is R²-10.364(=formula 1.3078).

Table 3079. Compounds of formula I.C, wherein R² is R²-10.365(=formula 1.3079).

Table 3080. Compounds of formula I.C, wherein R² is R²-10.366(=formula 1.3080).

Table 3081. Compounds of formula I.C, wherein R² is R²-10.367(=formula 1.3081).

Table 3082. Compounds of formula I.C, wherein R² is R²-10.368(=formula 1.3082).

Table 3083. Compounds of formula I.C, wherein R² is R²-10.369(=formula 1.3083).

Table 3084. Compounds of formula I.C, wherein R² is R²-10.370(=formula 1.3084).

Table 3085. Compounds of formula I.C, wherein R² is R²-10.371(=formula 1.3085).

Table 3086. Compounds of formula I.C, wherein R² is R²-10.372(=formula 1.3086).

Table 3087. Compounds of formula I.C, wherein R² is R²-10.373(=formula 1.3087).

Table 3088. Compounds of formula I.C, wherein R² is R²-10.374(=formula 1.3088).

Table 3089. Compounds of formula I.C, wherein R² is R²-10.375(=formula 1.3089).

Table 3090. Compounds of formula I.C, wherein R² is R²-10.376(=formula 1.3090).

Table 3091. Compounds of formula I.C, wherein R² is R²-10.377(=formula 1.3091).

Table 3092. Compounds of formula I.C, wherein R² is R²-10.378(=formula 1.3092).

Table 3093. Compounds of formula I.C, wherein R² is R²-10.379(=formula 1.3093).

Table 3094. Compounds of formula I.C, wherein R² is R²-10.380(=formula 1.3094).

Table 3095. Compounds of formula I.C, wherein R² is R²-10.381(=formula 1.3095).

Table 3096. Compounds of formula I.C, wherein R² is R²-10.382(=formula 1.3096).

Table 3097. Compounds of formula I.C, wherein R² is R²-10.383(=formula 1.3097).

Table 3098. Compounds of formula I.C, wherein R² is R²-10.384(=formula 1.3098).

Table 3099. Compounds of formula I.C, wherein R² is R²-10.385(=formula 1.3099).

Table 3100. Compounds of formula I.C, wherein R² is R²-10.386(=formula 1.3100).

Table 3101. Compounds of formula I.C, wherein R² is R²-10.387(=formula 1.3101).

Table 3102. Compounds of formula I.C, wherein R² is R²-10.388(=formula 1.3102).

Table 3103. Compounds of formula I.C, wherein R² is R²-10.389(=formula 1.3103).

Table 3104. Compounds of formula I.C, wherein R² is R²-10.390(=formula 1.3104).

Table 3105. Compounds of formula I.C, wherein R² is R²-10.391(=formula 1.3105).

Table 3106. Compounds of formula I.C, wherein R² is R²-10.392(=formula 1.3106).

Table 3107. Compounds of formula I.C, wherein R² is R²-10.393(=formula 1.3107).

Table 3108. Compounds of formula I.C, wherein R² is R²-10.394(=formula 1.3108).

Table 3109. Compounds of formula I.C, wherein R² is R²-10.395(=formula 1.3109).

Table 3110. Compounds of formula I.C, wherein R² is R²-10.396(=formula 1.3110).

Table 3111. Compounds of formula I.C, wherein R² is R²-10.397(=formula 1.3111).

Table 3112. Compounds of formula I.C, wherein R² is R²-10.398(=formula 1.3112).

Table 3113. Compounds of formula I.C, wherein R² is R²-10.399(=formula 1.3113).

Table 3114. Compounds of formula I.C, wherein R² is R²-10.400(=formula 1.3114).

Table 3115. Compounds of formula I.C, wherein R² is R²-10.401(=formula 1.3115).

Table 3116. Compounds of formula I.C, wherein R² is R²-10.402(=formula 1.3116).

Table 3117. Compounds of formula I.C, wherein R² is R²-10.403(=formula 1.3117).

Table 3118. Compounds of formula I.C, wherein R² is R²-10.404(=formula 1.3118).

Table 3119. Compounds of formula I.C, wherein R² is R²-10.405(=formula 1.3119).

Table 3120. Compounds of formula I.C, wherein R² is R²-10.406(=formula 1.3120).

Table 3121. Compounds of formula I.C, wherein R² is R²-10.407(=formula 1.3121).

Table 3122. Compounds of formula I.C, wherein R² is R²-10.408(=formula 1.3122).

Table 3123. Compounds of formula I.C, wherein R² is R²-10.409(=formula 1.3123).

Table 3124. Compounds of formula I.C, wherein R² is R²-10.410(=formula 1.3124).

Table 3125. Compounds of formula I.C, wherein R² is R²-10.411(=formula 1.3125).

Table 3126. Compounds of formula I.C, wherein R² is R²-10.412(=formula 1.3126).

Table 3127. Compounds of formula I.C, wherein R² is R²-10.413(=formula 1.3127).

Table 3128. Compounds of formula I.C, wherein R² is R²-10.414(=formula 1.3128).

Table 3129. Compounds of formula I.C, wherein R² is R²-10.415(=formula 1.3129).

Table 3130. Compounds of formula I.C, wherein R² is R²-10.416(=formula 1.3130).

Table 3131. Compounds of formula I.C, wherein R² is R²-10.417(=formula 1.3131).

Table 3132. Compounds of formula I.C, wherein R² is R²-10.418(=formula 1.3132).

Table 3133. Compounds of formula I.C, wherein R² is R²-10.419(=formula 1.3133).

Table 3134. Compounds of formula I.C, wherein R² is R²-10.420(=formula 1.3134).

Table 3135. Compounds of formula I.C, wherein R² is R²-10.421(=formula 1.3135).

Table 3136. Compounds of formula I.C, wherein R² is R²-10.422(=formula 1.3136).

Table 3137. Compounds of formula I.C, wherein R² is R²-10.423(=formula 1.3137).

Table 3138. Compounds of formula I.C, wherein R² is R²-10.424(=formula 1.3138).

Table 3139. Compounds of formula I.C, wherein R² is R²-10.425(=formula 1.3139).

Table 3140. Compounds of formula I.C, wherein R² is R²-10.426(=formula 1.3140).

Table 3141. Compounds of formula I.C, wherein R² is R²-10.427(=formula 1.3141).

Table 3142. Compounds of formula I.C, wherein R² is R²-10.428(=formula 1.3142).

Table 3143. Compounds of formula I.C, wherein R² is R²-10.429(=formula 1.3143).

Table 3144. Compounds of formula I.C, wherein R² is R²-10.430(=formula 1.3144).

Table 3145. Compounds of formula I.C, wherein R² is R²-10.431(=formula 1.3145).

Table 3146. Compounds of formula I.C, wherein R² is R²-10.432(=formula 1.3146).

Table 3147. Compounds of formula I.C, wherein R² is R²-10.433(=formula 1.3147).

Table 3148. Compounds of formula I.C, wherein R² is R²-10.434(=formula 1.3148).

Table 3149. Compounds of formula I.C, wherein R² is R²-10.435(=formula 1.3149).

Table 3150. Compounds of formula I.C, wherein R² is R²-10.436(=formula 1.3150).

Table 3151. Compounds of formula I.C, wherein R² is R²-10.437(=formula 1.3151).

Table 3152. Compounds of formula I.C, wherein R² is R²-10.438(=formula 1.3152).

Table 3153. Compounds of formula I.C, wherein R² is R²-10.439(=formula 1.3153).

Table 3154. Compounds of formula I.C, wherein R² is R²-10.440(=formula 1.3154).

Table 3155. Compounds of formula I.C, wherein R² is R²-10.441(=formula 1.3155).

Table 3156. Compounds of formula I.C, wherein R² is R²-10.442(=formula 1.3156).

Table 3157. Compounds of formula I.C, wherein R² is R²-10.443(=formula 1.3157).

Table 3158. Compounds of formula I.C, wherein R² is R²-10.444(=formula 1.3158).

Table 3159. Compounds of formula I.C, wherein R² is R²-10.445(=formula 1.3159).

Table 3160. Compounds of formula I.C, wherein R² is R²-10.446(=formula 1.3160).

Table 3161. Compounds of formula I.C, wherein R² is R²-10.447(=formula 1.3161).

Table 3162. Compounds of formula I.C, wherein R² is R²-10.448(=formula 1.3162).

Table 3163. Compounds of formula I.C, wherein R² is R²-10.449(=formula 1.3163).

Table 3164. Compounds of formula I.C, wherein R² is R²-10.450(=formula 1.3164).

Table 3165. Compounds of formula I.C, wherein R² is R²-10.451(=formula 1.3165).

Table 3166. Compounds of formula I.C, wherein R² is R²-10.452(=formula 1.3166).

Table 3167. Compounds of formula I.C, wherein R² is R²-10.453(=formula 1.3167).

Table 3168. Compounds of formula I.C, wherein R² is R²-10.454(=formula 1.3168).

Table 3169. Compounds of formula I.C, wherein R² is R²-10.455(=formula 1.3169).

Table 3170. Compounds of formula I.C, wherein R² is R²-10.456(=formula 1.3170).

Table 3171. Compounds of formula I.C, wherein R² is R²-10.457(=formula 1.3171).

Table 3172. Compounds of formula I.C, wherein R² is R²-10.458(=formula 1.3172).

Table 3173. Compounds of formula I.C, wherein R² is R²-10.459(=formula 1.3173).

Table 3174. Compounds of formula I.C, wherein R² is R²-10.460(=formula 1.3174).

Table 3175. Compounds of formula I.C, wherein R² is R²-10.461(=formula 1.3175).

Table 3176. Compounds of formula I.C, wherein R² is R²-10.462(=formula 1.3176).

Table 3177. Compounds of formula I.C, wherein R² is R²-10.463(=formula 1.3177).

Table 3178. Compounds of formula I.C, wherein R² is R²-10.464(=formula 1.3178).

Table 3179. Compounds of formula I.C, wherein R² is R²-10.465(=formula 1.3179).

Table 3180. Compounds of formula I.C, wherein R² is R²-10.466(=formula 1.3180).

Table 3181. Compounds of formula I.C, wherein R² is R²-10.467(=formula 1.3181).

Table 3182. Compounds of formula I.C, wherein R² is R²-10.468(=formula 1.3182).

Table 3183. Compounds of formula I.C, wherein R² is R²-10.469(=formula 1.3183).

Table 3184. Compounds of formula I.C, wherein R² is R²-10.470(=formula 1.3184).

Table 3185. Compounds of formula I.C, wherein R² is R²-10.471(=formula 1.3185).

Table 3186. Compounds of formula I.C, wherein R² is R²-10.472(=formula 1.3186).

Table 3187. Compounds of formula I.C, wherein R² is R²-10.473(=formula 1.3187).

Table 3188. Compounds of formula I.C, wherein R² is R²-10.474(=formula 1.3188).

Table 3189. Compounds of formula I.C, wherein R² is R²-10.475(=formula 1.3189).

Table 3190. Compounds of formula I.C, wherein R² is R²-10.476(=formula 1.3190).

Table 3191. Compounds of formula I.C, wherein R² is R²-10.477(=formula 1.3191).

Table 3192. Compounds of formula I.C, wherein R² is R²-10.478(=formula 1.3192).

Table 3193. Compounds of formula I.C, wherein R² is R²-10.479(=formula 1.3193).

Table 3194. Compounds of formula I.C, wherein R² is R²-10.480(=formula 1.3194).

Table 3195. Compounds of formula I.C, wherein R² is R²-10.481(=formula 1.3195).

Table 3196. Compounds of formula I.C, wherein R² is R²-10.482(=formula 1.3196).

Table 3197. Compounds of formula I.C, wherein R² is R²-10.483(=formula 1.3197).

Table 3198. Compounds of formula I.C, wherein R² is R²-10.484(=formula 1.3198).

Table 3199. Compounds of formula I.C, wherein R² is R²-10.485(=formula 1.3199).

Table 3200. Compounds of formula I.C, wherein R² is R²-10.486(=formula 1.3200).

Table 3201. Compounds of formula I.C, wherein R² is R²-10.487(=formula 1.3201).

Table 3202. Compounds of formula I.C, wherein R² is R²-10.488(=formula 1.3202).

Table 3203. Compounds of formula I.C, wherein R² is R²-10.489(=formula 1.3203).

Table 3204. Compounds of formula I.C, wherein R² is R²-10.490(=formula 1.3204).

Table 3205. Compounds of formula I.C, wherein R² is R²-10.491(=formula 1.3205).

Table 3206. Compounds of formula I.C, wherein R² is R²-10.492(=formula 1.3206).

Table 3207. Compounds of formula I.C, wherein R² is R²-10.493(=formula 1.3207).

Table 3208. Compounds of formula I.C, wherein R² is R²-10.494(=formula 1.3208).

Table 3209. Compounds of formula I.C, wherein R² is R²-10.495(=formula 1.3209).

Table 3210. Compounds of formula I.C, wherein R² is R²-10.496(=formula 1.3210).

Table 3211. Compounds of formula I.C, wherein R² is R²-10.497(=formula 1.3211).

Table 3212. Compounds of formula I.C, wherein R² is R²-10.498(=formula 1.3212).

Table 3213. Compounds of formula I.C, wherein R² is R²-10.499(=formula 1.3213).

Table 3214. Compounds of formula I.C, wherein R² is R²-10.500(=formula 1.3214).

Table 3215. Compounds of formula I.C, wherein R² is R²-10.501(=formula 1.3215).

Table 3216. Compounds of formula I.C, wherein R² is R²-10.502(=formula 1.3216).

Table 3217. Compounds of formula I.C, wherein R² is R²-10.503(=formula 1.3217).

Table 3218. Compounds of formula I.C, wherein R² is R²-10.504(=formula 1.3218).

Table 3219. Compounds of formula I.C, wherein R² is R²-10.505(=formula 1.3219).

Table 3220. Compounds of formula I.C, wherein R² is R²-10.506(=formula 1.3220).

Table 3221. Compounds of formula I.C, wherein R² is R²-10.507(=formula 1.3221).

Table 3222. Compounds of formula I.C, wherein R² is R²-10.508(=formula 1.3222).

Table 3223. Compounds of formula I.C, wherein R² is R²-10.509(=formula 1.3223).

Table 3224. Compounds of formula I.C, wherein R² is R²-10.510(=formula 1.3224).

Table 3225. Compounds of formula I.C, wherein R² is R²-10.511(=formula 1.3225).

Table 3226. Compounds of formula I.C, wherein R² is R²-10.512(=formula 1.3226).

Table 3227. Compounds of formula I.C, wherein R² is R²-10.513(=formula 1.3227).

Table 3228. Compounds of formula I.C, wherein R² is R²-10.514(=formula 1.3228).

Table 3229. Compounds of formula I.C, wherein R² is R²-10.515(=formula 1.3229).

Table 3230. Compounds of formula I.C, wherein R² is R²-10.516(=formula 1.3230).

Table 3231. Compounds of formula I.C, wherein R² is R²-10.517(=formula 1.3231).

Table 3232. Compounds of formula I.C, wherein R² is R²-10.518(=formula 1.3232).

Table 3233. Compounds of formula I.C, wherein R² is R²-10.519(=formula 1.3233).

Table 3234. Compounds of formula I.C, wherein R² is R²-10.520(=formula 1.3234).

Table 3235. Compounds of formula I.C, wherein R² is R²-10.521(=formula 1.3235).

Table 3236. Compounds of formula I.C, wherein R² is R²-10.522(=formula 1.3236).

Table 3237. Compounds of formula I.C, wherein R² is R²-10.523(=formula 1.3237).

Table 3238. Compounds of formula I.C, wherein R² is R²-10.524(=formula 1.3238).

Table 3239. Compounds of formula I.C, wherein R² is R²-10.525(=formula 1.3239).

Table 3240. Compounds of formula I.C, wherein R² is R²-10.526(=formula 1.3240).

Table 3241. Compounds of formula I.C, wherein R² is R²-10.527(=formula 1.3241).

Table 3242. Compounds of formula I.C, wherein R² is R²-10.528(=formula 1.3242).

Table 3243. Compounds of formula I.C, wherein R² is R²-10.529(=formula 1.3243).

Table 3244. Compounds of formula I.C, wherein R² is R²-10.530(=formula 1.3244).

Table 3245. Compounds of formula I.C, wherein R² is R²-10.531(=formula 1.3245).

Table 3246. Compounds of formula I.C, wherein R² is R²-10.532(=formula 1.3246).

Table 3247. Compounds of formula I.C, wherein R² is R²-10.533(=formula 1.3247).

Table 3248. Compounds of formula I.C, wherein R² is R²-10.534(=formula 1.3248).

Table 3249. Compounds of formula I.C, wherein R² is R²-10.535(=formula 1.3249).

Table 3250. Compounds of formula I.C, wherein R² is R²-10.536(=formula 1.3250).

Table 3251. Compounds of formula I.C, wherein R² is R²-10.537(=formula 1.3251).

Table 3252. Compounds of formula I.C, wherein R² is R²-10.538(=formula 1.3252).

Table 3253. Compounds of formula I.C, wherein R² is R²-10.539(=formula 1.3253).

Table 3254. Compounds of formula I.C, wherein R² is R²-10.540(=formula 1.3254).

Table 3255. Compounds of formula I.C, wherein R² is R²-10.541(=formula 1.3255).

Table 3256. Compounds of formula I.C, wherein R² is R²-10.542(=formula 1.3256).

Table 3257. Compounds of formula I.C, wherein R² is R²-10.543(=formula 1.3257).

Table 3258. Compounds of formula I.C, wherein R² is R²-10.544(=formula 1.3258).

Table 3259. Compounds of formula I.C, wherein R² is R²-10.545(=formula 1.3259).

Table 3260. Compounds of formula I.C, wherein R² is R²-10.546(=formula 1.3260).

Table 3261. Compounds of formula I.C, wherein R² is R²-10.547(=formula 1.3261).

Table 3262. Compounds of formula I.C, wherein R² is R²-10.548(=formula 1.3262).

Table 3263. Compounds of formula I.C, wherein R² is R²-10.549(=formula 1.3263.

Table 3264. Compounds of formula I.C, wherein R² is R²-10.550(=formula 1.3264).

Table 3265. Compounds of formula I.C, wherein R² is R²-10.551(=formula 1.3265).

Table 3266. Compounds of formula I.C, wherein R² is R²-10.552(=formula 1.3266).

Table 3267. Compounds of formula I.C, wherein R² is R²-10.553(=formula 1.3267).

Table 3268. Compounds of formula I.C, wherein R² is R²-10.554(=formula 1.3268).

Table 3269. Compounds of formula I.C, wherein R² is R²-10.555(=formula 1.3269).

Table 3270. Compounds of formula I.C, wherein R² is R²-10.556(=formula 1.3270).

Table 3271. Compounds of formula I.C, wherein R² is R²-10.557(=formula 1.3271).

Table 3272. Compounds of formula I.C, wherein R² is R²-10.558(=formula 1.3272).

Table 3273. Compounds of formula I.C, wherein R² is R²-10.559(=formula 1.3273).

Table 3274. Compounds of formula I.C, wherein R² is R²-10.560(=formula 1.3274).

Table 3275. Compounds of formula I.C, wherein R² is R²-10.561(=formula 1.3275).

Table 3276. Compounds of formula I.C, wherein R² is R²-10.562(=formula 1.3276).

Table 3277. Compounds of formula I.C, wherein R² is R²-10.563(=formula 1.3277).

Table 3278. Compounds of formula I.C, wherein R² is R²-10.564(=formula 1.3278).

Table 3279. Compounds of formula I.C, wherein R² is R²-10.565(=formula 1.3279).

Table 3280. Compounds of formula I.C, wherein R² is R²-10.566(=formula 1.3280).

Table 3281. Compounds of formula I.C, wherein R² is R²-10.567(=formula 1.3281).

Table 3282. Compounds of formula I.C, wherein R² is R²-10.568(=formula 1.3282).

Table 3283. Compounds of formula I.C, wherein R² is R²-10.569(=formula 1.3283).

Table 3284. Compounds of formula I.C, wherein R² is R²-10.570(=formula 1.3284).

Table 3285. Compounds of formula I.C, wherein R² is R²-10.571(=formula 1.3285).

Table 3286. Compounds of formula I.C, wherein R² is R²-10.572(=formula 1.3286).

Table 3287. Compounds of formula I.C, wherein R² is R²-10.573(=formula 1.3287).

Table 3288. Compounds of formula I.C, wherein R² is R²-10.574(=formula 1.3288).

Table 3289. Compounds of formula I.C, wherein R² is R²-10.575(=formula 1.3289).

Table 3290. Compounds of formula I.C, wherein R² is R²-10.576(=formula 1.3290).

Table 3291. Compounds of formula I.C, wherein R² is R²-10.577(=formula 1.3291).

Table 3292. Compounds of formula I.C, wherein R² is R²-10.578(=formula 1.3292).

Table 3293. Compounds of formula I.C, wherein R² is R²-10.579(=formula 1.3293).

Table 3294. Compounds of formula I.C, wherein R² is R²-10.580(=formula 1.3294).

Table 3295. Compounds of formula I.C, wherein R² is R²-10.581(=formula 1.3295).

Table 3296. Compounds of formula I.C, wherein R² is R²-10.582(=formula 1.3296).

Table 3297. Compounds of formula I.C, wherein R² is R²-10.583(=formula 1.3297).

Table 3298. Compounds of formula I.C, wherein R² is R²-10.584(=formula 1.3298).

Table 3299. Compounds of formula I.C, wherein R² is R²-10.585(=formula 1.3299).

Table 3300. Compounds of formula I.C, wherein R² is R²-10.586(=formula 1.3300).

Table 3301. Compounds of formula I.C, wherein R² is R²-10.587(=formula 1.3301).

Table 3302. Compounds of formula I.C, wherein R² is R²-10.588(=formula 1.3302).

Table 3303. Compounds of formula I.C, wherein R² is R²-10.589(=formula 1.3303).

Table 3304. Compounds of formula I.C, wherein R² is R²-10.590(=formula 1.3304).

Table 3305. Compounds of formula I.C, wherein R² is R²-10.591(=formula 1.3305).

Table 3306. Compounds of formula I.C, wherein R² is R²-10.592(=formula 1.3306).

Table 3307. Compounds of formula I.C, wherein R² is R²-10.593(=formula 1.3307).

Table 3308. Compounds of formula I.C, wherein R² is R²-10.594(=formula 1.3308).

Table 3309. Compounds of formula I.C, wherein R² is R²-10.595(=formula 1.3309).

Table 3310. Compounds of formula I.C, wherein R² is R²-10.596(=formula 1.3310).

Table 3311. Compounds of formula I.C, wherein R² is R²-10.597(=formula 1.3311).

Table 3312. Compounds of formula I.C, wherein R² is R²-10.598(=formula 1.3312).

Table 3313. Compounds of formula I.C, wherein R² is R²-10.599(=formula 1.3313).

Table 3314. Compounds of formula I.C, wherein R² is R²-10.600(=formula 1.3314).

Table 3315. Compounds of formula I.C, wherein R² is R²-10.601(=formula 1.3315).

Table 3316. Compounds of formula I.C, wherein R² is R²-10.602(=formula 1.3316).

Table 3317. Compounds of formula I.C, wherein R² is R²-10.603(=formula 1.3317).

Table 3318. Compounds of formula I.C, wherein R² is R²-10.604(=formula 1.3318).

Table 3319. Compounds of formula I.C, wherein R² is R²-10.605(=formula 1.3319).

Table 3320. Compounds of formula I.C, wherein R² is R²-10.606(=formula 1.3320).

Table 3321. Compounds of formula I.C, wherein R² is R²-10.607(=formula 1.3321).

Table 3322. Compounds of formula I.C, wherein R² is R²-10.608(=formula 1.3322).

Table 3323. Compounds of formula I.C, wherein R² is R²-10.609(=formula 1.3323).

Table 3324. Compounds of formula I.C, wherein R² is R²-10.610(=formula 1.3324).

Table 3325. Compounds of formula I.C, wherein R² is R²-10.611(=formula 1.3325).

Table 3326. Compounds of formula I.C, wherein R² is R²-10.612(=formula 1.3326).

Table 3327. Compounds of formula I.C, wherein R² is R²-10.613(=formula 1.3327).

Table 3328. Compounds of formula I.C, wherein R² is R²-10.614(=formula 1.3328).

Table 3329. Compounds of formula I.C, wherein R² is R²-10.615(=formula 1.3329).

Table 3330. Compounds of formula I.C, wherein R² is R²-10.616(=formula 1.3330).

Table 3331. Compounds of formula I.C, wherein R² is R²-10.617(=formula 1.3331).

Table 3332. Compounds of formula I.C, wherein R² is R²-10.618(=formula 1.3332).

Table 3333. Compounds of formula I.C, wherein R² is R²-10.619(=formula 1.3333).

Table 3334. Compounds of formula I.C, wherein R² is R²-10.620(=formula 1.3334).

Table 3335. Compounds of formula I.C, wherein R² is R²-10.621(=formula 1.3335).

Table 3336. Compounds of formula I.C, wherein R² is R²-10.622(=formula 1.3336).

Table 3337. Compounds of formula I.C, wherein R² is R²-10.623(=formula 1.3337).

Table 3338. Compounds of formula I.C, wherein R² is R²-10.624(=formula 1.3338).

Table 3339. Compounds of formula I.C, wherein R² is R²-10.625(=formula 1.3339).

Table 3340. Compounds of formula I.C, wherein R² is R²-10.626(=formula 1.3340).

Table 3341. Compounds of formula I.C, wherein R² is R²-10.627(=formula 1.3341).

Table 3342. Compounds of formula I.C, wherein R² is R²-10.628(=formula 1.3342).

Table 3343. Compounds of formula I.C, wherein R² is R²-10.629(=formula 1.3343).

Table 3344. Compounds of formula I.C, wherein R² is R²-10.630(=formula 1.3344).

Table 3345. Compounds of formula I.C, wherein R² is R²-10.631(=formula 1.3345).

Table 3346. Compounds of formula I.C, wherein R² is R²-10.632(=formula 1.3346).

Table 3347. Compounds of formula I.C, wherein R² is R²-10.633(=formula 1.3347).

Table 3348. Compounds of formula I.C, wherein R² is R²-10.634(=formula 1.3348).

Table 3349. Compounds of formula I.C, wherein R² is R²-10.635(=formula 1.3349).

Table 3350. Compounds of formula I.C, wherein R² is R²-10.636(=formula 1.3350).

Table 3351. Compounds of formula I.C, wherein R² is R²-10.637(=formula 1.3351).

Table 3352. Compounds of formula I.C, wherein R² is R²-10.638(=formula 1.3352).

Table 3353. Compounds of formula I.C, wherein R² is R²-10.639(=formula 1.3353).

Table 3354. Compounds of formula I.C, wherein R² is R²-10.640(=formula 1.3354).

Table 3355. Compounds of formula I.C, wherein R² is R²-10.641(=formula 1.3355).

Table 3356. Compounds of formula I.C, wherein R² is R²-10.642(=formula 1.3356).

Table 3357. Compounds of formula I.C, wherein R² is R²-10.643(=formula 1.3357).

Table 3358. Compounds of formula I.C, wherein R² is R²-10.644(=formula 1.3358).

Table 3359. Compounds of formula I.C, wherein R² is R²-10.645(=formula 1.3359).

Table 3360. Compounds of formula I.C, wherein R² is R²-10.646(=formula 1.3360).

Table 3361. Compounds of formula I.C, wherein R² is R²-10.647(=formula 1.3361).

Table 3362. Compounds of formula I.C, wherein R² is R²-10.648(=formula 1.3362).

Table 3363. Compounds of formula I.C, wherein R² is R²-10.649(=formula 1.3363).

Table 3364. Compounds of formula I.C, wherein R² is R²-10.650(=formula 1.3364).

Table 3365. Compounds of formula I.C, wherein R² is R²-10.651(=formula 1.3365).

Table 3366. Compounds of formula I.C, wherein R² is R²-10.652(=formula 1.3366).

Table 3367. Compounds of formula I.C, wherein R² is R²-10.653(=formula 1.3367).

Table 3368. Compounds of formula I.C, wherein R² is R²-10.654(=formula 1.3368).

Table 3369. Compounds of formula I.C, wherein R² is R²-10.655(=formula 1.3369).

Table 3370. Compounds of formula I.C, wherein R² is R²-10.656(=formula 1.3370).

Table 3371. Compounds of formula I.C, wherein R² is R²-10.657(=formula 1.3371).

Table 3372. Compounds of formula I.C, wherein R² is R²-10.658(=formula 1.3372).

Table 3373. Compounds of formula I.C, wherein R² is R²-10.659(=formula 1.3373).

Table 3374. Compounds of formula I.C, wherein R² is R²-10.660(=formula 1.3374).

Table 3375. Compounds of formula I.C, wherein R² is R²-10.661(=formula 1.3375).

Table 3376. Compounds of formula I.C, wherein R² is R²-10.662(=formula 1.3376).

Table 3377. Compounds of formula I.C, wherein R² is R²-10.663(=formula 1.3377).

Table 3378. Compounds of formula I.C, wherein R² is R²-10.664(=formula 1.3378).

Table 3379. Compounds of formula I.C, wherein R² is R²-10.665(=formula 1.3379).

Table 3380. Compounds of formula I.C, wherein R² is R²-10.666(=formula 1.3380).

Table 3381. Compounds of formula I.C, wherein R² is R²-10.667(=formula 1.3381).

Table 3382. Compounds of formula I.C, wherein R² is R²-10.668(=formula 1.3382).

Table 3383. Compounds of formula I.C, wherein R² is R²-10.669(=formula 1.3383).

Table 3384. Compounds of formula I.C, wherein R² is R²-10.670(=formula 1.3384).

Table 3385. Compounds of formula I.C, wherein R² is R²-10.671(=formula 1.3385).

Table 3386. Compounds of formula I.C, wherein R² is R²-10.672(=formula 1.3386).

Table 3387. Compounds of formula I.C, wherein R² is R²-10.673(=formula 1.3387).

Table 3388. Compounds of formula I.C, wherein R² is R²-10.674(=formula 1.3388).

Table 3389. Compounds of formula I.C, wherein R² is R²-10.675(=formula 1.3389).

Table 3390. Compounds of formula I.C, wherein R² is R²-10.676(=formula 1.3390).

Table 3391. Compounds of formula I.C, wherein R² is R²-15.1(=formula 1.3391).

Table 3392. Compounds of formula I.C, wherein R² is R²-15.2(=formula 1.3392).

Table 3393. Compounds of formula I.C, wherein R² is R²-15.3(=formula 1.3393).

Table 3394. Compounds of formula I.C, wherein R² is R²-15.4(=formula 1.3394).

Table 3395. Compounds of formula I.C, wherein R² is R²-15.5(=formula 1.3395).

Table 3396. Compounds of formula I.C, wherein R² is R²-15.6(=formula 1.3396).

Table 3397. Compounds of formula I.C, wherein R² is R²-15.7(=formula 1.3397).

Table 3398. Compounds of formula I.C, wherein R² is R²-15.8(=formula 1.3398).

Table 3399. Compounds of formula I.C, wherein R² is R²-15.9(=formula 1.3399).

Table 3400. Compounds of formula I.C, wherein R² is R²-15.10(=formula 1.3400).

Table 3401. Compounds of formula I.C, wherein R² is R²-15.11(=formula 1.3401).

Table 3402. Compounds of formula I.C, wherein R² is R²-15.12(=formula 1.3402).

Table 3403. Compounds of formula I.C, wherein R² is R²-15.13(=formula 1.3403).

Table 3404. Compounds of formula I.C, wherein R² is R²-15.14(=formula 1.3404).

Table 3405. Compounds of formula I.C, wherein R² is R²-15.15(=formula 1.3405).

Table 3406. Compounds of formula I.C, wherein R² is R²-15.16(=formula 1.3406).

Table 3407. Compounds of formula I.C, wherein R² is R²-15.17(=formula 1.3407).

Table 3408. Compounds of formula I.C, wherein R² is R²-15.18(=formula 1.3408).

Table 3409. Compounds of formula I.C, wherein R² is R²-15.19(=formula 1.3409).

Table 3410. Compounds of formula I.C, wherein R² is R²-15.20(=formula 1.3410).

Table 3411. Compounds of formula I.C, wherein R² is R²-15.21(=formula 1.3411).

Table 3412. Compounds of formula I.C, wherein R² is R²-15.22(=formula 1.3412).

Table 3413. Compounds of formula I.C, wherein R² is R²-15.23(=formula 1.3413).

Table 3414. Compounds of formula I.C, wherein R² is R²-15.24(=formula 1.3414).

Table 3415. Compounds of formula I.C, wherein R² is R²-15.25(=formula 1.3415).

Table 3416. Compounds of formula I.C, wherein R² is R²-15.26(=formula 1.3416).

Table 3417. Compounds of formula I.C, wherein R² is R²-15.27(=formula 1.3417).

Table 3418. Compounds of formula I.C, wherein R² is R²-15.28(=formula 1.3418).

Table 3419. Compounds of formula I.C, wherein R² is R²-15.29(=formula 1.3419).

Table 3420. Compounds of formula I.C, wherein R² is R²-15.30(=formula 1.3420).

Table 3421. Compounds of formula I.C, wherein R² is R²-15.31(=formula 1.3421).

Table 3422. Compounds of formula I.C, wherein R² is R²-15.32(=formula 1.3422).

Table 3423. Compounds of formula I.C, wherein R² is R²-15.33(=formula 1.3423).

Table 3424. Compounds of formula I.C, wherein R² is R²-15.34(=formula 1.3424).

Table 3425. Compounds of formula I.C, wherein R² is R²-15.35(=formula 1.3425).

Table 3426. Compounds of formula I.C, wherein R² is R²-15.36(=formula 1.3426).

Table 3427. Compounds of formula I.C, wherein R² is R²-15.37(=formula 1.3427).

Table 3428. Compounds of formula I.C, wherein R² is R²-15.38(=formula 1.3428).

Table 3429. Compounds of formula I.C, wherein R² is R²-15.39(=formula 1.3429).

Table 3430. Compounds of formula I.C, wherein R² is R²-15.40(=formula 1.3430).

Table 3431. Compounds of formula I.C, wherein R² is R²-15.41(=formula 1.3431).

Table 3432. Compounds of formula I.C, wherein R² is R²-15.42(=formula 1.3432).

Table 3433. Compounds of formula I.C, wherein R² is R²-15.43(=formula 1.3433).

Table 3434. Compounds of formula I.C, wherein R² is R²-15.44(=formula 1.3434).

Table 3435. Compounds of formula I.C, wherein R² is R²-15.45(=formula 1.3435).

Table 3436. Compounds of formula I.C, wherein R² is R²-15.46(=formula 1.3436).

Table 3437. Compounds of formula I.C, wherein R² is R²-15.47(=formula 1.3437).

Table 3438. Compounds of formula I.C, wherein R² is R²-15.48(=formula 1.3438).

Table 3439. Compounds of formula I.C, wherein R² is R²-15.49(=formula 1.3439).

Table 3440. Compounds of formula I.C, wherein R² is R²-15.50(=formula 1.3440).

Table 3441. Compounds of formula I.C, wherein R² is R²-15.51(=formula 1.3441).

Table 3442. Compounds of formula I.C, wherein R² is R²-15.52(=formula 1.3442).

Table 3443. Compounds of formula I.C, wherein R² is R²-15.53(=formula 1.3443).

Table 3444. Compounds of formula I.C, wherein R² is R²-15.54(=formula 1.3444).

Table 3445. Compounds of formula I.C, wherein R² is R²-15.55(=formula 1.3445).

Table 3446. Compounds of formula I.C, wherein R² is R²-15.56(=formula 1.3446).

Table 3447. Compounds of formula I.C, wherein R² is R²-15.57(=formula 1.3447).

Table 3448. Compounds of formula I.C, wherein R² is R²-15.58(=formula 1.3448).

Table 3449. Compounds of formula I.C, wherein R² is R²-15.59(=formula 1.3449).

Table 3450. Compounds of formula I.C, wherein R² is R²-15.60(=formula 1.3450).

Table 3451. Compounds of formula I.C, wherein R² is R²-15.61(=formula 1.3451).

Table 3452. Compounds of formula I.C, wherein R² is R²-15.62(=formula 1.3452).

Table 3453. Compounds of formula I.C, wherein R² is R²-15.63(=formula 1.3453).

Table 3454. Compounds of formula I.C, wherein R² is R²-15.64(=formula 1.3454).

Table 3455. Compounds of formula I.C, wherein R² is R²-15.65(=formula 1.3455).

Table 3456. Compounds of formula I.C, wherein R² is R²-15.66(=formula 1.3456).

Table 3457. Compounds of formula I.C, wherein R² is R²-15.67(=formula 1.3457).

Table 3458. Compounds of formula I.C, wherein R² is R²-15.68(=formula 1.3458).

Table 3459. Compounds of formula I.C, wherein R² is R²-15.69(=formula 1.3459).

Table 3460. Compounds of formula I.C, wherein R² is R²-15.70(=formula 1.3460).

Table 3461. Compounds of formula I.C, wherein R² is R²-15.71(=formula 1.3461).

Table 3462. Compounds of formula I.C, wherein R² is R²-15.72(=formula 1.3462).

Table 3463. Compounds of formula I.C, wherein R² is R²-15.73(=formula 1.3463).

Table 3464. Compounds of formula I.C, wherein R² is R²-15.74(=formula 1.3464).

Table 3465. Compounds of formula I.C, wherein R² is R²-15.75(=formula 1.3465).

Table 3466. Compounds of formula I.C, wherein R² is R²-15.76(=formula 1.3466).

Table 3467. Compounds of formula I.C, wherein R² is R²-15.77(=formula 1.3467).

Table 3468. Compounds of formula I.C, wherein R² is R²-15.78(=formula 1.3468).

Table 3469. Compounds of formula I.C, wherein R² is R²-15.79(=formula 1.3469).

Table 3470. Compounds of formula I.C, wherein R² is R²-15.80(=formula 1.3470).

Table 3471. Compounds of formula I.C, wherein R² is R²-15.81(=formula 1.3471).

Table 3472. Compounds of formula I.C, wherein R² is R²-15.82(=formula 1.3472).

Table 3473. Compounds of formula I.C, wherein R² is R²-15.83(=formula 1.3473).

Table 3474. Compounds of formula I.C, wherein R² is R²-15.84(=formula 1.3474).

Table 3475. Compounds of formula I.C, wherein R² is R²-15.85(=formula 1.3475).

Table 3476. Compounds of formula I.C, wherein R² is R²-15.86(=formula 1.3476).

Table 3477. Compounds of formula I.C, wherein R² is R²-15.87(=formula 1.3477).

Table 3478. Compounds of formula I.C, wherein R² is R²-15.88(=formula 1.3478).

Table 3479. Compounds of formula I.C, wherein R² is R²-15.89(=formula 1.3479).

Table 3480. Compounds of formula I.C, wherein R² is R²-15.90(=formula 1.3480).

Table 3481. Compounds of formula I.C, wherein R² is R²-15.91(=formula 1.3481).

Table 3482. Compounds of formula I.C, wherein R² is R²-15.92(=formula 1.3482).

Table 3483. Compounds of formula I.C, wherein R² is R²-15.93(=formula 1.3483).

Table 3484. Compounds of formula I.C, wherein R² is R²-15.94(=formula 1.3484).

Table 3485. Compounds of formula I.C, wherein R² is R²-15.95(=formula 1.3485).

Table 3486. Compounds of formula I.C, wherein R² is R²-15.96(=formula 1.3486).

Table 3487. Compounds of formula I.C, wherein R² is R²-15.97(=formula 1.3487).

Table 3488. Compounds of formula I.C, wherein R² is R²-15.98(=formula 1.3488).

Table 3489. Compounds of formula I.C, wherein R² is R²-15.99(=formula 1.3489).

Table 3490. Compounds of formula I.C, wherein R² is R²-15.100(=formula 1.3490).

Table 3491. Compounds of formula I.C, wherein R² is R²-15.101(=formula 1.3491).

Table 3492. Compounds of formula I.C, wherein R² is R²-15.102(=formula 1.3492).

Table 3493. Compounds of formula I.C, wherein R² is R²-15.103(=formula 1.3493).

Table 3494. Compounds of formula I.C, wherein R² is R²-15.104(=formula 1.3494).

Table 3495. Compounds of formula I.C, wherein R² is R²-15.105(=formula 1.3495).

Table 3496. Compounds of formula I.C, wherein R² is R²-15.106(=formula 1.3496).

Table 3497. Compounds of formula I.C, wherein R² is R²-15.107(=formula 1.3497).

Table 3498. Compounds of formula I.C, wherein R² is R²-15.108(=formula 1.3498).

Table 3499. Compounds of formula I.C, wherein R² is R²-15.109(=formula 1.3499).

Table 3500. Compounds of formula I.C, wherein R² is R²-15.110(=formula 1.3500).

Table 3501. Compounds of formula I.C, wherein R² is R²-15.111(=formula 1.3501).

Table 3502. Compounds of formula I.C, wherein R² is R²-15.112(=formula 1.3502).

Table 3503. Compounds of formula I.C, wherein R² is R²-15.113(=formula 1.3503).

Table 3504. Compounds of formula I.C, wherein R² is R²-15.114(=formula 1.3504).

Table 3505. Compounds of formula I.C, wherein R² is R²-15.115(=formula 1.3505).

Table 3506. Compounds of formula I.C, wherein R² is R²-15.116(=formula 1.3506).

Table 3507. Compounds of formula I.C, wherein R² is R²-15.117(=formula 1.3507).

Table 3508. Compounds of formula I.C, wherein R² is R²-15.118(=formula 1.3508).

Table 3509. Compounds of formula I.C, wherein R² is R²-15.119(=formula 1.3509).

Table 3510. Compounds of formula I.C, wherein R² is R²-15.120(=formula 1.3510).

Table 3511. Compounds of formula I.C, wherein R² is R²-15.121(=formula 1.3511).

Table 3512. Compounds of formula I.C, wherein R² is R²-15.122(=formula 1.3512).

Table 3513. Compounds of formula I.C, wherein R² is R²-15.123(=formula 1.3513).

Table 3514. Compounds of formula I.C, wherein R² is R²-15.124(=formula 1.3514).

Table 3515. Compounds of formula I.C, wherein R² is R²-15.125(=formula 1.3515).

Table 3516. Compounds of formula I.C, wherein R² is R²-15.126(=formula 1.3516).

Table 3517. Compounds of formula I.C, wherein R² is R²-15.127(=formula 1.3517).

Table 3518. Compounds of formula I.C, wherein R² is R²-15.128(=formula 1.3518).

Table 3519. Compounds of formula I.C, wherein R² is R²-15.129(=formula 1.3519).

Table 3520. Compounds of formula I.C, wherein R² is R²-15.130(=formula 1.3520).

Table 3521. Compounds of formula I.C, wherein R² is R²-15.131(=formula 1.3521).

Table 3522. Compounds of formula I.C, wherein R² is R²-15.132(=formula 1.3522).

Table 3523. Compounds of formula I.C, wherein R² is R²-15.133(=formula 1.3523).

Table 3524. Compounds of formula I.C, wherein R² is R²-15.134(=formula 1.3524).

Table 3525. Compounds of formula I.C, wherein R² is R²-15.135(=formula 1.3525).

Table 3526. Compounds of formula I.C, wherein R² is R²-15.136(=formula 1.3526).

Table 3527. Compounds of formula I.C, wherein R² is R²-15.137(=formula 1.3527).

Table 3528. Compounds of formula I.C, wherein R² is R²-15.138(=formula 1.3528).

Table 3529. Compounds of formula I.C, wherein R² is R²-15.139(=formula 1.3529).

Table 3530. Compounds of formula I.C, wherein R² is R²-15.140(=formula 1.3530).

Table 3531. Compounds of formula I.C, wherein R² is R²-15.141(=formula 1.3531).

Table 3532. Compounds of formula I.C, wherein R² is R²-15.142(=formula 1.3532).

Table 3533. Compounds of formula I.C, wherein R² is R²-15.143(=formula 1.3533).

Table 3534. Compounds of formula I.C, wherein R² is R²-15.144(=formula 1.3534).

Table 3535. Compounds of formula I.C, wherein R² is R²-15.145(=formula 1.3535).

Table 3536. Compounds of formula I.C, wherein R² is R²-15.146(=formula 1.3536).

Table 3537. Compounds of formula I.C, wherein R² is R²-15.147(=formula 1.3537).

Table 3538. Compounds of formula I.C, wherein R² is R²-15.148(=formula 1.3538).

Table 3539. Compounds of formula I.C, wherein R² is R²-15.149(=formula 1.3539).

Table 3540. Compounds of formula I.C, wherein R² is R²-15.150(=formula 1.3540).

Table 3541. Compounds of formula I.C, wherein R² is R²-15.151(=formula 1.3541).

Table 3542. Compounds of formula I.C, wherein R² is R²-15.152(=formula 1.3542).

Table 3543. Compounds of formula I.C, wherein R² is R²-15.153(=formula 1.3543).

Table 3544. Compounds of formula I.C, wherein R² is R²-15.154(=formula 1.3544).

Table 3545. Compounds of formula I.C, wherein R² is R²-15.155(=formula 1.3545).

Table 3546. Compounds of formula I.C, wherein R² is R²-15.156(=formula 1.3546).

Table 3547. Compounds of formula I.C, wherein R² is R²-15.157(=formula 1.3547).

Table 3548. Compounds of formula I.C, wherein R² is R²-15.158(=formula 1.3548).

Table 3549. Compounds of formula I.C, wherein R² is R²-15.159(=formula 1.3549).

Table 3550. Compounds of formula I.C, wherein R² is R²-15.160(=formula 1.3550).

Table 3551. Compounds of formula I.C, wherein R² is R²-15.161(=formula 1.3551).

Table 3552. Compounds of formula I.C, wherein R² is R²-15.162(=formula 1.3552).

Table 3553. Compounds of formula I.C, wherein R² is R²-15.163(=formula 1.3553).

Table 3554. Compounds of formula I.C, wherein R² is R²-15.164(=formula 1.3554).

Table 3555. Compounds of formula I.C, wherein R² is R²-15.165(=formula 1.3555).

Table 3556. Compounds of formula I.C, wherein R² is R²-15.166(=formula 1.3556).

Table 3557. Compounds of formula I.C, wherein R² is R²-15.167(=formula 1.3557).

Table 3558. Compounds of formula I.C, wherein R² is R²-15.168(=formula 1.3558).

Table 3559. Compounds of formula I.C, wherein R² is R²-15.169(=formula 1.3559).

Table 3560. Compounds of formula I.C, wherein R² is R²-15.170(=formula 1.3560).

Table 3561. Compounds of formula I.C, wherein R² is R²-15.171(=formula 1.3561).

Table 3562. Compounds of formula I.C, wherein R² is R²-15.172(=formula 1.3562).

Table 3563. Compounds of formula I.C, wherein R² is R²-15.173(=formula 1.3563).

Table 3564. Compounds of formula I.C, wherein R² is R²-15.174(=formula 1.3564).

Table 3565. Compounds of formula I.C, wherein R² is R²-15.175(=formula 1.3565).

Table 3566. Compounds of formula I.C, wherein R² is R²-15.176(=formula 1.3566).

Table 3567. Compounds of formula I.C, wherein R² is R²-15.177(=formula 1.3567).

Table 3568. Compounds of formula I.C, wherein R² is R²-15.178(=formula 1.3568).

Table 3569. Compounds of formula I.C, wherein R² is R²-15.179(=formula 1.3569).

Table 3570. Compounds of formula I.C, wherein R² is R²-15.180(=formula 1.3570).

Table 3571. Compounds of formula I.C, wherein R² is R²-15.181(=formula 1.3571).

Table 3572. Compounds of formula I.C, wherein R² is R²-15.182(=formula 1.3572).

Table 3573. Compounds of formula I.C, wherein R² is R²-15.183(=formula 1.3573).

Table 3574. Compounds of formula I.C, wherein R² is R²-15.184(=formula 1.3574).

Table 3575. Compounds of formula I.C, wherein R² is R²-15.185(=formula 1.3575).

Table 3576. Compounds of formula I.C, wherein R² is R²-15.186(=formula 1.3576).

Table 3577. Compounds of formula I.C, wherein R² is R²-15.187(=formula 1.3577).

Table 3578. Compounds of formula I.C, wherein R² is R²-15.188(=formula 1.3578).

Table 3579. Compounds of formula I.C, wherein R² is R²-15.189(=formula 1.3579).

Table 3580. Compounds of formula I.C, wherein R² is R²-15.190(=formula 1.3580).

Table 3581. Compounds of formula I.C, wherein R² is R²-15.191(=formula 1.3581).

Table 3582. Compounds of formula I.C, wherein R² is R²-15.192(=formula 1.3582).

Table 3583. Compounds of formula I.C, wherein R² is R²-15.193(=formula 1.3583).

Table 3584. Compounds of formula I.C, wherein R² is R²-15.194(=formula 1.3584).

Table 3585. Compounds of formula I.C, wherein R² is R²-15.195(=formula 1.3585).

Table 3586. Compounds of formula I.C, wherein R² is R²-15.196(=formula 1.3586).

Table 3587. Compounds of formula I.C, wherein R² is R²-15.197(=formula 1.3587).

Table 3588. Compounds of formula I.C, wherein R² is R²-15.198(=formula 1.3588).

Table 3589. Compounds of formula I.C, wherein R² is R²-15.199(=formula 1.3589).

Table 3590. Compounds of formula I.C, wherein R² is R²-15.200(=formula 1.3590).

Table 3591. Compounds of formula I.C, wherein R² is R²-15.201(=formula 1.3591).

Table 3592. Compounds of formula I.C, wherein R² is R²-15.202(=formula 1.3592).

Table 3593. Compounds of formula I.C, wherein R² is R²-15.203(=formula 1.3593).

Table 3594. Compounds of formula I.C, wherein R² is R²-15.204(=formula 1.3594).

Table 3595. Compounds of formula I.C, wherein R² is R²-15.205(=formula 1.3595).

Table 3596. Compounds of formula I.C, wherein R² is R²-15.206(=formula 1.3596).

Table 3597. Compounds of formula I.C, wherein R² is R²-15.207(=formula 1.3597).

Table 3598. Compounds of formula I.C, wherein R² is R²-15.208(=formula 1.3598).

Table 3599. Compounds of formula I.C, wherein R² is R²-15.209(=formula 1.3599).

Table 3600. Compounds of formula I.C, wherein R² is R²-15.210(=formula 1.3600).

Table 3601. Compounds of formula I.C, wherein R² is R²-15.211(=formula 1.3601).

Table 3602. Compounds of formula I.C, wherein R² is R²-15.212(=formula 1.3602).

Table 3603. Compounds of formula I.C, wherein R² is R²-15.213(=formula 1.3603).

Table 3604. Compounds of formula I.C, wherein R² is R²-15.214(=formula 1.3604).

Table 3605. Compounds of formula I.C, wherein R² is R²-15.215(=formula 1.3605).

Table 3606. Compounds of formula I.C, wherein R² is R²-15.216(=formula 1.3606).

Table 3607. Compounds of formula I.C, wherein R² is R²-15.217(=formula 1.3607).

Table 3608. Compounds of formula I.C, wherein R² is R²-15.218(=formula 1.3608).

Table 3609. Compounds of formula I.C, wherein R² is R²-15.219(=formula 1.3609).

Table 3610. Compounds of formula I.C, wherein R² is R²-15.220(=formula 1.3610).

Table 3611. Compounds of formula I.C, wherein R² is R²-15.221(=formula 1.3611).

Table 3612. Compounds of formula I.C, wherein R² is R²-15.222(=formula 1.3612).

Table 3613. Compounds of formula I.C, wherein R² is R²-15.223(=formula 1.3613).

Table 3614. Compounds of formula I.C, wherein R² is R²-15.224(=formula 1.3614).

Table 3615. Compounds of formula I.C, wherein R² is R²-15.225(=formula 1.3645).

Table 3616. Compounds of formula I.C, wherein R² is R²-15.226(=formula 1.3646).

Table 3617. Compounds of formula I.C, wherein R² is R²-15.227(=formula 1.3617).

Table 3618. Compounds of formula I.C, wherein R² is R²-15.228(=formula 1.3618).

Table 3619. Compounds of formula I.C, wherein R² is R²-15.229(=formula 1.3619).

Table 3620. Compounds of formula I.C, wherein R² is R²-15.230(=formula 1.3620).

Table 3621. Compounds of formula I.C, wherein R² is R²-15.231(=formula 1.3621).

Table 3622. Compounds of formula I.C, wherein R² is R²-15.232(=formula 1.3622).

Table 3623. Compounds of formula I.C, wherein R² is R²-15.233(=formula 1.3623).

Table 3624. Compounds of formula I.C, wherein R² is R²-15.234(=formula 1.3624).

Table 3625. Compounds of formula I.C, wherein R² is R²-15.235(=formula 1.3625).

Table 3626. Compounds of formula I.C, wherein R² is R²-15.236(=formula 1.3626).

Table 3627. Compounds of formula I.C, wherein R² is R²-15.237(=formula 1.3627).

Table 3628. Compounds of formula I.C, wherein R² is R²-15.238(=formula 1.3628).

Table 3629. Compounds of formula I.C, wherein R² is R²-15.239(=formula 1.3629).

Table 3630. Compounds of formula I.C, wherein R² is R²-15.240(=formula 1.3630).

Table 3631. Compounds of formula I.C, wherein R² is R²-15.241(=formula 1.3631).

Table 3632. Compounds of formula I.C, wherein R² is R²-15.242(=formula 1.3632).

Table 3633. Compounds of formula I.C, wherein R² is R²-15.243(=formula 1.3633).

Table 3634. Compounds of formula I.C, wherein R² is R²-15.244(=formula 1.3634).

Table 3635. Compounds of formula I.C, wherein R² is R²-15.245(=formula 1.3635).

Table 3636. Compounds of formula I.C, wherein R² is R²-15.246(=formula 1.3636).

Table 3637. Compounds of formula I.C, wherein R² is R²-15.247(=formula 1.3637).

Table 3638. Compounds of formula I.C, wherein R² is R²-15.248(=formula 1.3638).

Table 3639. Compounds of formula I.C, wherein R² is R²-15.249(=formula 1.3639).

Table 3640. Compounds of formula I.C, wherein R² is R²-15.250(=formula 1.3640).

Table 3641. Compounds of formula I.C, wherein R² is R²-15.251(=formula 1.3641).

Table 3642. Compounds of formula I.C, wherein R² is R²-15.252(=formula 1.3642).

Table 3643. Compounds of formula I.C, wherein R² is R²-15.253(=formula 1.3643).

Table 3644. Compounds of formula I.C, wherein R² is R²-15.254(=formula 1.3644).

Table 3645. Compounds of formula I.C, wherein R² is R²-15.255(=formula 1.3645).

Table 3646. Compounds of formula I.C, wherein R² is R²-15.256(=formula 1.3646).

Table 3647. Compounds of formula I.C, wherein R² is R²-15.257(=formula 1.3647).

Table 3648. Compounds of formula I.C, wherein R² is R²-15.258(=formula 1.3648).

Table 3649. Compounds of formula I.C, wherein R² is R²-15.259(=formula 1.3649).

Table 3650. Compounds of formula I.C, wherein R² is R²-15.260(=formula 1.3650).

Table 3651. Compounds of formula I.C, wherein R² is R²-15.261(=formula 1.3651).

Table 3652. Compounds of formula I.C, wherein R² is R²-15.262(=formula 1.3652).

Table 3653. Compounds of formula I.C, wherein R² is R²-15.263(=formula 1.3653).

Table 3654. Compounds of formula I.C, wherein R² is R²-15.264(=formula 1.3654).

Table 3655. Compounds of formula I.C, wherein R² is R²-15.265(=formula 1.3655).

Table 3656. Compounds of formula I.C, wherein R² is R²-15.266(=formula 1.3656).

Table 3657. Compounds of formula I.C, wherein R² is R²-15.267(=formula 1.3657).

Table 3658. Compounds of formula I.C, wherein R² is R²-15.268(=formula 1.3658).

Table 3659. Compounds of formula I.C, wherein R² is R²-15.269(=formula 1.3659).

Table 3660. Compounds of formula I.C, wherein R² is R²-15.270(=formula 1.3660).

Table 3661. Compounds of formula I.C, wherein R² is R²-15.271(=formula 1.3661).

Table 3662. Compounds of formula I.C, wherein R² is R²-15.272(=formula 1.3662).

Table 3663. Compounds of formula I.C, wherein R² is R²-15.273(=formula 1.3663).

Table 3664. Compounds of formula I.C, wherein R² is R²-15.274(=formula 1.3664).

Table 3665. Compounds of formula I.C, wherein R² is R²-15.275(=formula 1.3665).

Table 3666. Compounds of formula I.C, wherein R² is R²-15.276(=formula 1.3666).

Table 3667. Compounds of formula I.C, wherein R² is R²-15.277(=formula 1.3667).

Table 3668. Compounds of formula I.C, wherein R² is R²-15.278(=formula 1.3668).

Table 3669. Compounds of formula I.C, wherein R² is R²-15.279(=formula 1.3669).

Table 3670. Compounds of formula I.C, wherein R² is R²-15.280(=formula 1.3670).

Table 3671. Compounds of formula I.C, wherein R² is R²-15.281(=formula 1.3671).

Table 3672. Compounds of formula I.C, wherein R² is R²-15.282(=formula 1.3672).

Table 3673. Compounds of formula I.C, wherein R² is R²-15.283(=formula 1.3673).

Table 3674. Compounds of formula I.C, wherein R² is R²-15.284(=formula 1.3674).

Table 3675. Compounds of formula I.C, wherein R² is R²-15.285(=formula 1.3675).

Table 3676. Compounds of formula I.C, wherein R² is R²-15.286(=formula 1.3676).

Table 3677. Compounds of formula I.C, wherein R² is R²-15.287(=formula 1.3677).

Table 3678. Compounds of formula I.C, wherein R² is R²-15.288(=formula 1.3678).

Table 3679. Compounds of formula I.C, wherein R² is R²-15.289(=formula 1.3679).

Table 3680. Compounds of formula I.C, wherein R² is R²-15.290(=formula 1.3680).

Table 3681. Compounds of formula I.C, wherein R² is R²-15.291(=formula 1.3681).

Table 3682. Compounds of formula I.C, wherein R² is R²-15.292(=formula 1.3682).

Table 3683. Compounds of formula I.C, wherein R² is R²-15.293(=formula 1.3683).

Table 3684. Compounds of formula I.C, wherein R² is R²-15.294(=formula 1.3684).

Table 3685. Compounds of formula I.C, wherein R² is R²-15.295(=formula 1.3685).

Table 3686. Compounds of formula I.C, wherein R² is R²-15.296(=formula 1.3686).

Table 3687. Compounds of formula I.C, wherein R² is R²-15.297(=formula 1.3687).

Table 3688. Compounds of formula I.C, wherein R² is R²-15.298(=formula 1.3688).

Table 3689. Compounds of formula I.C, wherein R² is R²-15.299(=formula 1.3689).

Table 3690. Compounds of formula I.C, wherein R² is R²-15.300(=formula 1.3690).

Table 3691. Compounds of formula I.C, wherein R² is R²-15.301(=formula 1.3691).

Table 3692. Compounds of formula I.C, wherein R² is R²-15.302(=formula 1.3692).

Table 3693. Compounds of formula I.C, wherein R² is R²-15.303(=formula 1.3693).

Table 3694. Compounds of formula I.C, wherein R² is R²-15.304(=formula 1.3694).

Table 3695. Compounds of formula I.C, wherein R² is R²-15.305(=formula 1.3695).

Table 3696. Compounds of formula I.C, wherein R² is R²-15.306(=formula 1.3696).

Table 3697. Compounds of formula I.C, wherein R² is R²-15.307(=formula 1.3697).

Table 3698. Compounds of formula I.C, wherein R² is R²-15.308(=formula 1.3698).

Table 3699. Compounds of formula I.C, wherein R² is R²-15.309(=formula 1.3699).

Table 3700. Compounds of formula I.C, wherein R² is R²-15.310(=formula 1.3700).

Table 3701. Compounds of formula I.C, wherein R² is R²-15.311(=formula 1.3701).

Table 3702. Compounds of formula I.C, wherein R² is R²-15.312(=formula 1.3702).

Table 3703. Compounds of formula I.C, wherein R² is R²-15.313(=formula 1.3703).

Table 3704. Compounds of formula I.C, wherein R² is R²-15.314(=formula 1.3704).

Table 3705. Compounds of formula I.C, wherein R² is R²-15.315(=formula 1.3705).

Table 3706. Compounds of formula I.C, wherein R² is R²-15.316(=formula 1.3706).

Table 3707. Compounds of formula I.C, wherein R² is R²-15.317(=formula 1.3707).

Table 3708. Compounds of formula I.C, wherein R² is R²-15.318(=formula 1.3708).

Table 3709. Compounds of formula I.C, wherein R² is R²-15.319(=formula 1.3709).

Table 3710. Compounds of formula I.C, wherein R² is R²-15.320(=formula 1.3710).

Table 3711. Compounds of formula I.C, wherein R² is R²-15.321(=formula 1.3711).

Table 3712. Compounds of formula I.C, wherein R² is R²-15.322(=formula 1.3712).

Table 3713. Compounds of formula I.C, wherein R² is R²-15.323(=formula 1.3713).

Table 3714. Compounds of formula I.C, wherein R² is R²-15.324(=formula 1.3714).

Table 3715. Compounds of formula I.C, wherein R² is R²-15.325(=formula 1.3715).

Table 3716. Compounds of formula I.C, wherein R² is R²-15.326(=formula 1.3716).

Table 3717. Compounds of formula I.C, wherein R² is R²-15.327(=formula 1.3717).

Table 3718. Compounds of formula I.C, wherein R² is R²-15.328(=formula 1.3718).

Table 3719. Compounds of formula I.C, wherein R² is R²-15.329(=formula 1.3719).

Table 3720. Compounds of formula I.C, wherein R² is R²-15.330(=formula 1.3720).

Table 3721. Compounds of formula I.C, wherein R² is R²-15.331(=formula 1.3721).

Table 3722. Compounds of formula I.C, wherein R² is R²-15.332(=formula 1.3722).

Table 3723. Compounds of formula I.C, wherein R² is R²-15.333(=formula 1.3723).

Table 3724. Compounds of formula I.C, wherein R² is R²-15.334(=formula 1.3724).

Table 3725. Compounds of formula I.C, wherein R² is R²-15.335(=formula 1.3725).

Table 3726. Compounds of formula I.C, wherein R² is R²-15.336(=formula 1.3726).

Table 3727. Compounds of formula I.C, wherein R² is R²-15.334(=formula 1.3727).

Table 3728. Compounds of formula I.C, wherein R² is R²-15.338(=formula 1.3728).

Table 3729. Compounds of formula I.C, wherein R² is R²-15.339(=formula 1.3729).

Table 3730. Compounds of formula I.C, wherein R² is R²-15.340(=formula 1.3730).

Table 3731. Compounds of formula I.C, wherein R² is R²-15.341(=formula 1.3731).

Table 3732. Compounds of formula I.C, wherein R² is R²-15.342(=formula 1.3732).

Table 3733. Compounds of formula I.C, wherein R² is R²-15.343(=formula 1.3733).

Table 3734. Compounds of formula I.C, wherein R² is R²-15.344(=formula 1.3734).

Table 3735. Compounds of formula I.C, wherein R² is R²-15.345(=formula 1.3735).

Table 3736. Compounds of formula I.C, wherein R² is R²-15.346(=formula 1.3736).

Table 3737. Compounds of formula I.C, wherein R² is R²-15.347(=formula 1.3737).

Table 3738. Compounds of formula I.C, wherein R² is R²-15.348(=formula 1.3738).

Table 3739. Compounds of formula I.C, wherein R² is R²-15.349(=formula 1.3739).

Table 3740. Compounds of formula I.C, wherein R² is R²-15.350(=formula 1.3740).

Table 3741. Compounds of formula I.C, wherein R² is R²-15.351(=formula 1.3741).

Table 3742. Compounds of formula I.C, wherein R² is R²-15.352(=formula 1.3742).

Table 3743. Compounds of formula I.C, wherein R² is R²-15.353(=formula 1.3743).

Table 3744. Compounds of formula I.C, wherein R² is R²-15.354(=formula 1.3744).

Table 3745. Compounds of formula I.C, wherein R² is R²-15.355(=formula 1.3745).

Table 3746. Compounds of formula I.C, wherein R² is R²-15.356(=formula 1.3746).

Table 3747. Compounds of formula I.C, wherein R² is R²-15.357(=formula 1.3747).

Table 3748. Compounds of formula I.C, wherein R² is R²-15.358(=formula 1.3748).

Table 3749. Compounds of formula I.C, wherein R² is R²-15.359(=formula 1.3749).

Table 3750. Compounds of formula I.C, wherein R² is R²-15.360(=formula 1.3750).

Table 3751. Compounds of formula I.C, wherein R² is R²-15.361(=formula 1.3751).

Table 3752. Compounds of formula I.C, wherein R² is R²-15.362(=formula 1.3752).

Table 3753. Compounds of formula I.C, wherein R² is R²-15.363(=formula 1.3753).

Table 3754. Compounds of formula I.C, wherein R² is R²-15.364(=formula 1.3754).

Table 3755. Compounds of formula I.C, wherein R² is R²-15.365(=formula 1.3755).

Table 3756. Compounds of formula I.C, wherein R² is R²-15.366(=formula 1.3756).

Table 3757. Compounds of formula I.C, wherein R² is R²-15.367(=formula 1.3757).

Table 3758. Compounds of formula I.C, wherein R² is R²-15.368(=formula 1.3758).

Table 3759. Compounds of formula I.C, wherein R² is R²-15.369(=formula 1.3759).

Table 3760. Compounds of formula I.C, wherein R² is R²-15.370(=formula 1.3760).

Table 3761. Compounds of formula I.C, wherein R² is R²-15.371(=formula 1.3761).

Table 3762. Compounds of formula I.C, wherein R² is R²-15.372(=formula 1.3762).

Table 3763. Compounds of formula I.C, wherein R² is R²-15.373(=formula 1.3763).

Table 3764. Compounds of formula I.C, wherein R² is R²-15.374(=formula 1.3764).

Table 3765. Compounds of formula I.C, wherein R² is R²-15.375(=formula 1.3765).

Table 3766. Compounds of formula I.C, wherein R² is R²-15.376(=formula 1.3766).

Table 3767. Compounds of formula I.C, wherein R² is R²-15.377(=formula 1.3767).

Table 3768. Compounds of formula I.C, wherein R² is R²-15.378(=formula 1.3768).

Table 3769. Compounds of formula I.C, wherein R² is R²-15.379(=formula 1.3769).

Table 3770. Compounds of formula I.C, wherein R² is R²-15.380(=formula 1.3770).

Table 3771. Compounds of formula I.C, wherein R² is R²-15.381(=formula 1.3771).

Table 3772. Compounds of formula I.C, wherein R² is R²-15.382(=formula 1.3772).

Table 3773. Compounds of formula I.C, wherein R² is R²-15.383(=formula 1.3773).

Table 3774. Compounds of formula I.C, wherein R² is R²-15.384(=formula 1.3774).

Table 3775. Compounds of formula I.C, wherein R² is R²-15.385(=formula 1.3775).

Table 3776. Compounds of formula I.C, wherein R² is R²-15.386(=formula 1.3776.

Table 3777. Compounds of formula I.C, wherein R² is R²-15.387(=formula 1.3777).

Table 3778. Compounds of formula I.C, wherein R² is R²-15.388(=formula 1.3778).

Table 3779. Compounds of formula I.C, wherein R² is R²-15.389(=formula 1.3779).

Table 3780. Compounds of formula I.C, wherein R² is R²-15.390(=formula 1.3780).

Table 3781. Compounds of formula I.C, wherein R² is R²-15.391(=formula 1.3781).

Table 3782. Compounds of formula I.C, wherein R² is R²-15.392(=formula 1.3782).

Table 3783. Compounds of formula I.C, wherein R² is R²-15.393(=formula 1.3783).

Table 3784. Compounds of formula I.C, wherein R² is R²-15.394(=formula 1.3784).

Table 3785. Compounds of formula I.C, wherein R² is R²-15.395(=formula 1.3785).

Table 3786. Compounds of formula I.C, wherein R² is R²-15.396(=formula 1.3786).

Table 3787. Compounds of formula I.C, wherein R² is R²-15.397(=formula 1.3787).

Table 3788. Compounds of formula I.C, wherein R² is R²-15.398(=formula 1.3788).

Table 3789. Compounds of formula I.C, wherein R² is R²-15.399(=formula 1.3789).

Table 3790. Compounds of formula I.C, wherein R² is R²-15.400(=formula 1.3790).

Table 3791. Compounds of formula I.C, wherein R² is R²-15.401(=formula 1.3791).

Table 3792. Compounds of formula I.C, wherein R² is R²-15.402(=formula 1.3792).

Table 3793. Compounds of formula I.C, wherein R² is R²-15.403(=formula 1.3793).

Table 3794. Compounds of formula I.C, wherein R² is R²-15.404(=formula 1.3794).

Table 3795. Compounds of formula I.C, wherein R² is R²-15.405(=formula 1.3795).

Table 3796. Compounds of formula I.C, wherein R² is R²-15.406(=formula 1.3796).

Table 3797. Compounds of formula I.C, wherein R² is R²-15.407(=formula 1.3797).

Table 3798. Compounds of formula I.C, wherein R² is R²-15.408(=formula 1.3798).

Table 3799. Compounds of formula I.C, wherein R² is R²-15.409(=formula 1.3799).

Table 3800. Compounds of formula I.C, wherein R² is R²-15.410(=formula 1.3800).

Table 3801. Compounds of formula I.C, wherein R² is R²-15.411(=formula 1.3801).

Table 3802. Compounds of formula I.C, wherein R² is R²-15.412(=formula 1.3802).

Table 3803. Compounds of formula I.C, wherein R² is R²-15.413(=formula 1.3803).

Table 3804. Compounds of formula I.C, wherein R² is R²-15.414(=formula 1.3804).

Table 3805. Compounds of formula I.C, wherein R² is R²-15.415(=formula 1.3805).

Table 3806. Compounds of formula I.C, wherein R² is R²-15.416(=formula 1.3806).

Table 3807. Compounds of formula I.C, wherein R² is R²-15.417(=formula 1.3807).

Table 3808. Compounds of formula I.C, wherein R² is R²-15.418(=formula 1.3808).

Table 3809. Compounds of formula I.C, wherein R² is R²-15.419(=formula 1.3809).

Table 3810. Compounds of formula I.C, wherein R² is R²-15.420(=formula 1.3810).

Table 3811. Compounds of formula I.C, wherein R² is R²-15.421(=formula 1.3811).

Table 3812. Compounds of formula I.C, wherein R² is R²-15.422(=formula 1.3812).

Table 3813. Compounds of formula I.C, wherein R² is R²-15.423(=formula 1.3813).

Table 3814. Compounds of formula I.C, wherein R² is R²-15.424(=formula 1.3814).

Table 3815. Compounds of formula I.C, wherein R² is R²-15.425(=formula 1.3815).

Table 3816. Compounds of formula I.C, wherein R² is R²-15.426(=formula 1.3816).

Table 3817. Compounds of formula I.C, wherein R² is R²-15.427(=formula 1.3817).

Table 3818. Compounds of formula I.C, wherein R² is R²-15.428(=formula 1.3818).

Table 3819. Compounds of formula I.C, wherein R² is R²-15.429(=formula 1.3819).

Table 3820. Compounds of formula I.C, wherein R² is R²-15.430(=formula 1.3820).

Table 3821. Compounds of formula I.C, wherein R² is R²-15.431(=formula 1.3821).

Table 3822. Compounds of formula I.C, wherein R² is R²-15.432(=formula 1.3822).

Table 3823. Compounds of formula I.C, wherein R² is R²-15.433(=formula 1.3823).

Table 3824. Compounds of formula I.C, wherein R² is R²-15.434(=formula 1.3824).

Table 3825. Compounds of formula I.C, wherein R² is R²-15.435(=formula 1.3825).

Table 3826. Compounds of formula I.C, wherein R² is R²-15.436(=formula 1.3826).

Table 3827. Compounds of formula I.C, wherein R² is R²-15.437(=formula 1.3827).

Table 3828. Compounds of formula I.C, wherein R² is R²-15.438(=formula 1.3828).

Table 3829. Compounds of formula I.C, wherein R² is R²-15.439(=formula 1.3829).

Table 3830. Compounds of formula I.C, wherein R² is R²-15.440(=formula 1.3830).

Table 3831. Compounds of formula I.C, wherein R² is R²-15.441(=formula 1.3831).

Table 3832. Compounds of formula I.C, wherein R² is R²-15.442(=formula 1.3832).

Table 3833. Compounds of formula I.C, wherein R² is R²-15.443(=formula 1.3833).

Table 3834. Compounds of formula I.C, wherein R² is R²-15.444(=formula 1.3834).

Table 3835. Compounds of formula I.C, wherein R² is R²-15.445(=formula 1.3835).

Table 3836. Compounds of formula I.C, wherein R² is R²-15.446(=formula 1.3836).

Table 3837. Compounds of formula I.C, wherein R² is R²-15.447(=formula 1.3837).

Table 3838. Compounds of formula I.C, wherein R² is R²-15.448(=formula 1.3838).

Table 3839. Compounds of formula I.C, wherein R² is R²-15.449(=formula 1.3839).

Table 3840. Compounds of formula I.C, wherein R² is R²-15.450(=formula 1.3840).

Table 3841. Compounds of formula I.C, wherein R² is R²-15.451(=formula 1.3841).

Table 3842. Compounds of formula I.C, wherein R² is R²-15.452(=formula 1.3842).

Table 3843. Compounds of formula I.C, wherein R² is R²-15.453(=formula 1.3843).

Table 3844. Compounds of formula I.C, wherein R² is R²-15.454(=formula 1.3844).

Table 3845. Compounds of formula I.C, wherein R² is R²-15.455(=formula 1.3845).

Table 3846. Compounds of formula I.C, wherein R² is R²-15.456(=formula 1.3846).

Table 3847. Compounds of formula I.C, wherein R² is R²-15.457(=formula 1.3847).

Table 3848. Compounds of formula I.C, wherein R² is R²-15.458(=formula 1.3848).

Table 3849. Compounds of formula I.C, wherein R² is R²-15.459(=formula 1.3849).

Table 3850. Compounds of formula I.C, wherein R² is R²-15.460(=formula 1.3850).

Table 3851. Compounds of formula I.C, wherein R² is R²-15.461(=formula 1.3851).

Table 3852. Compounds of formula I.C, wherein R² is R²-15.462(=formula 1.3852).

Table 3853. Compounds of formula I.C, wherein R² is R²-15.463(=formula 1.3853).

Table 3854. Compounds of formula I.C, wherein R² is R²-15.464(=formula 1.3854).

Table 3855. Compounds of formula I.C, wherein R² is R²-15.465(=formula 1.3855).

Table 3856. Compounds of formula I.C, wherein R² is R²-15.466(=formula 1.3856).

Table 3857. Compounds of formula I.C, wherein R² is R²-15.467(=formula 1.3857).

Table 3858. Compounds of formula I.C, wherein R² is R²-15.468(=formula 1.3858).

Table 3859. Compounds of formula I.C, wherein R² is R²-15.469(=formula 1.3859).

Table 3860. Compounds of formula I.C, wherein R² is R²-15.470(=formula 1.3860).

Table 3861. Compounds of formula I.C, wherein R² is R²-15.471(=formula 1.3861).

Table 3862. Compounds of formula I.C, wherein R² is R²-15.472(=formula 1.3862).

Table 3863. Compounds of formula I.C, wherein R² is R²-15.473(=formula 1.3863).

Table 3864. Compounds of formula I.C, wherein R² is R²-15.474(=formula 1.3864).

Table 3865. Compounds of formula I.C, wherein R² is R²-15.475(=formula 1.3865).

Table 3866. Compounds of formula I.C, wherein R² is R²-15.476(=formula 1.3866).

Table 3867. Compounds of formula I.C, wherein R² is R²-15.477(=formula 1.3867).

Table 3868. Compounds of formula I.C, wherein R² is R²-15.478(=formula 1.3868).

Table 3869. Compounds of formula I.C, wherein R² is R²-15.479(=formula 1.3869).

Table 3870. Compounds of formula I.C, wherein R² is R²-15.480(=formula 1.3870).

Table 3871. Compounds of formula I.C, wherein R² is R²-15.481(=formula 1.3871).

Table 3872. Compounds of formula I.C, wherein R² is R²-15.482(=formula 1.3872).

Table 3873. Compounds of formula I.C, wherein R² is R²-15.483(=formula 1.3873).

Table 3874. Compounds of formula I.C, wherein R² is R²-15.484(=formula 1.3874).

Table 3875. Compounds of formula I.C, wherein R² is R²-15.485(=formula 1.3875).

Table 3876. Compounds of formula I.C, wherein R² is R²-15.486(=formula 1.3876).

Table 3877. Compounds of formula I.C, wherein R² is R²-15.487(=formula 1.3877).

Table 3878. Compounds of formula I.C, wherein R² is R²-15.488(=formula 1.3878).

Table 3879. Compounds of formula I.C, wherein R² is R²-15.489(=formula 1.3879).

Table 3880. Compounds of formula I.C, wherein R² is R²-15.490(=formula 1.3880).

Table 3881. Compounds of formula I.C, wherein R² is R²-15.491(=formula 1.3881).

Table 3882. Compounds of formula I.C, wherein R² is R²-15.492(=formula 1.3882).

Table 3883. Compounds of formula I.C, wherein R² is R²-15.493(=formula 1.3883).

Table 3884. Compounds of formula I.C, wherein R² is R²-15.494(=formula 1.3884).

Table 3885. Compounds of formula I.C, wherein R² is R²-15.495(=formula 1.3885).

Table 3886. Compounds of formula I.C, wherein R² is R²-15.496(=formula 1.3886).

Table 3887. Compounds of formula I.C, wherein R² is R²-15.497(=formula 1.3887).

Table 3888. Compounds of formula I.C, wherein R² is R²-15.498(=formula 1.3888).

Table 3889. Compounds of formula I.C, wherein R² is R²-15.499(=formula 1.3889).

Table 3890. Compounds of formula I.C, wherein R² is R²-15.500(=formula 1.3890).

Table 3891. Compounds of formula I.C, wherein R² is R²-15.501(=formula 1.3891).

Table 3892. Compounds of formula I.C, wherein R² is R²-15.502(=formula 1.3892).

Table 3893. Compounds of formula I.C, wherein R² is R²-15.503(=formula 1.3893).

Table 3894. Compounds of formula I.C, wherein R² is R²-15.504(=formula 1.3894).

Table 3895. Compounds of formula I.C, wherein R² is R²-15.505(=formula 1.3895).

Table 3896. Compounds of formula I.C, wherein R² is R²-15.506(=formula 1.3896).

Table 3897. Compounds of formula I.C, wherein R² is R²-15.507(=formula 1.3897).

Table 3898. Compounds of formula I.C, wherein R² is R²-15.508(=formula 1.3898).

Table 3899. Compounds of formula I.C, wherein R² is R²-15.509(=formula 1.3899).

Table 3900. Compounds of formula I.C, wherein R² is R²-15.510(=formula 1.3900).

Table 3901. Compounds of formula I.C, wherein R² is R²-15.511(=formula 1.3901).

Table 3902. Compounds of formula I.C, wherein R² is R²-15.512(=formula 1.3902).

Table 3903. Compounds of formula I.C, wherein R² is R²-15.513(=formula 1.3903).

Table 3904. Compounds of formula I.C, wherein R² is R²-15.514(=formula 1.3904).

Table 3905. Compounds of formula I.C, wherein R² is R²-15.515(=formula 1.3905).

Table 3906. Compounds of formula I.C, wherein R² is R²-15.516(=formula 1.3906).

Table 3907. Compounds of formula I.C, wherein R² is R²-15.517(=formula 1.3907).

Table 3908. Compounds of formula I.C, wherein R² is R²-15.518(=formula 1.3908).

Table 3909. Compounds of formula I.C, wherein R² is R²-15.519(=formula 1.3909).

Table 3910. Compounds of formula I.C, wherein R² is R²-15.520(=formula 1.3910).

Table 3911. Compounds of formula I.C, wherein R² is R²-15.521(=formula 1.3911).

Table 3912. Compounds of formula I.C, wherein R² is R²-15.522(=formula 1.3912).

Table 3913. Compounds of formula I.C, wherein R² is R²-15.523(=formula 1.3913).

Table 3914. Compounds of formula I.C, wherein R² is R²-15.524(=formula 1.3914).

Table 3915. Compounds of formula I.C, wherein R² is R²-15.525(=formula 1.3915).

Table 3916. Compounds of formula I.C, wherein R² is R²-15.526(=formula 1.3916).

Table 3917. Compounds of formula I.C, wherein R² is R²-15.527(=formula 1.3917).

Table 3918. Compounds of formula I.C, wherein R² is R²-15.528(=formula 1.3918).

Table 3919. Compounds of formula I.C, wherein R² is R²-15.529(=formula 1.3919).

Table 3920. Compounds of formula I.C, wherein R² is R²-15.530(=formula 1.3920).

Table 3921. Compounds of formula I.C, wherein R² is R²-15.531(=formula 1.3921).

Table 3922. Compounds of formula I.C, wherein R² is R²-15.532(=formula 1.3922).

Table 3923. Compounds of formula I.C, wherein R² is R²-15.533(=formula 1.3923).

Table 3924. Compounds of formula I.C, wherein R² is R²-15.534(=formula 1.3924).

Table 3925. Compounds of formula I.C, wherein R² is R²-15.535(=formula 1.3925).

Table 3926. Compounds of formula I.C, wherein R² is R²-15.536(=formula 1.3926).

Table 3927. Compounds of formula I.C, wherein R² is R²-15.537(=formula 1.3927).

Table 3928. Compounds of formula I.C, wherein R² is R²-15.538(=formula 1.3928).

Table 3929. Compounds of formula I.C, wherein R² is R²-15.539(=formula 1.3929).

Table 3930. Compounds of formula I.C, wherein R² is R²-15.540(=formula 1.3930).

Table 3931. Compounds of formula I.C, wherein R² is R²-15.541(=formula 1.3931).

Table 3932. Compounds of formula I.C, wherein R² is R²-15.542(=formula 1.3932).

Table 3933. Compounds of formula I.C, wherein R² is R²-15.543(=formula 1.3933).

Table 3934. Compounds of formula I.C, wherein R² is R²-15.544(=formula 1.3934).

Table 3935. Compounds of formula I.C, wherein R² is R²-15.545(=formula 1.3935).

Table 3936. Compounds of formula I.C, wherein R² is R²-15.546(=formula 1.3936).

Table 3937. Compounds of formula I.C, wherein R² is R²-15.547(=formula 1.3937).

Table 3938. Compounds of formula I.C, wherein R² is R²-15.548(=formula 1.3938).

Table 3939. Compounds of formula I.C, wherein R² is R²-15.549(=formula 1.3939).

Table 3940. Compounds of formula I.C, wherein R² is R²-15.550(=formula 1.3940).

Table 3941. Compounds of formula I.C, wherein R² is R²-15.551(=formula 1.3941).

Table 3942. Compounds of formula I.C, wherein R² is R²-15.552(=formula 1.3942).

Table 3943. Compounds of formula I.C, wherein R² is R²-15.553(=formula 1.3943).

Table 3944. Compounds of formula I.C, wherein R² is R²-15.554(=formula 1.3944).

Table 3945. Compounds of formula I.C, wherein R² is R²-15.555(=formula 1.3945).

Table 3946. Compounds of formula I.C, wherein R² is R²-15.556(=formula 1.3946).

Table 3947. Compounds of formula I.C, wherein R² is R²-15.557(=formula 1.3947).

Table 3948. Compounds of formula I.C, wherein R² is R²-15.558(=formula 1.3948).

Table 3949. Compounds of formula I.C, wherein R² is R²-15.559(=formula 1.3949).

Table 3950. Compounds of formula I.C, wherein R² is R²-15.560(=formula 1.3950).

Table 3951. Compounds of formula I.C, wherein R² is R²-15.561(=formula 1.3951).

Table 3952. Compounds of formula I.C, wherein R² is R²-15.562(=formula 1.3952).

Table 3953. Compounds of formula I.C, wherein R² is R²-15.563(=formula 1.3953).

Table 3954. Compounds of formula I.C, wherein R² is R²-15.564(=formula 1.3954).

Table 3955. Compounds of formula I.C, wherein R² is R²-15.565(=formula 1.3955).

Table 3956. Compounds of formula I.C, wherein R² is R²-15.566(=formula 1.3956).

Table 3957. Compounds of formula I.C, wherein R² is R²-15.567(=formula 1.3957).

Table 3958. Compounds of formula I.C, wherein R² is R²-15.568(=formula 1.3958).

Table 3959. Compounds of formula I.C, wherein R² is R²-15.569(=formula 1.3959).

Table 3960. Compounds of formula I.C, wherein R² is R²-15.570(=formula 1.3960).

Table 3961. Compounds of formula I.C, wherein R² is R²-15.571(=formula 1.3961).

Table 3962. Compounds of formula I.C, wherein R² is R²-15.572(=formula 1.3962).

Table 3963. Compounds of formula I.C, wherein R² is R²-15.573(=formula 1.3963).

Table 3964. Compounds of formula I.C, wherein R² is R²-15.574(=formula 1.3964).

Table 3965. Compounds of formula I.C, wherein R² is R²-15.575(=formula 1.3965).

Table 3966. Compounds of formula I.C, wherein R² is R²-15.576(=formula 1.3966).

Table 3967. Compounds of formula I.C, wherein R² is R²-15.577(=formula 1.3967).

Table 3968. Compounds of formula I.C, wherein R² is R²-15.578(=formula 1.3968).

Table 3969. Compounds of formula I.C, wherein R² is R²-15.579(=formula 1.3969).

Table 3970. Compounds of formula I.C, wherein R² is R²-15.580(=formula 1.3970).

Table 3971. Compounds of formula I.C, wherein R² is R²-15.581(=formula 1.3971).

Table 3972. Compounds of formula I.C, wherein R² is R²-15.582(=formula 1.3972).

Table 3973. Compounds of formula I.C, wherein R² is R²-15.583(=formula 1.3973).

Table 3974. Compounds of formula I.C, wherein R² is R²-15.584(=formula 1.3974).

Table 3975. Compounds of formula I.C, wherein R² is R²-15.585(=formula 1.3975).

Table 3976. Compounds of formula I.C, wherein R² is R²-15.586(=formula 1.3976).

Table 3977. Compounds of formula I.C, wherein R² is R²-15.587(=formula 1.3977).

Table 3978. Compounds of formula I.C, wherein R² is R²-15.588(=formula 1.3978).

Table 3979. Compounds of formula I.C, wherein R² is R²-15.589(=formula 1.3979).

Table 3980. Compounds of formula I.C, wherein R² is R²-15.590(=formula 1.3980).

Table 3981. Compounds of formula I.C, wherein R² is R²-15.591(=formula 1.3981).

Table 3982. Compounds of formula I.C, wherein R² is R²-15.592(=formula 1.3982).

Table 3983. Compounds of formula I.C, wherein R² is R²-15.593(=formula 1.3983).

Table 3984. Compounds of formula I.C, wherein R² is R²-15.594(=formula 1.3984).

Table 3985. Compounds of formula I.C, wherein R² is R²-15.595(=formula 1.3985).

Table 3986. Compounds of formula I.C, wherein R² is R²-15.596(=formula 1.3986).

Table 3987. Compounds of formula I.C, wherein R² is R²-15.597(=formula 1.3987).

Table 3988. Compounds of formula I.C, wherein R² is R²-15.598(=formula 1.3988).

Table 3989. Compounds of formula I.C, wherein R² is R²-15.599(=formula 1.3989).

Table 3990. Compounds of formula I.C, wherein R² is R²-15.600(=formula 1.3990).

Table 3991. Compounds of formula I.C, wherein R² is R²-15.601(=formula 1.3991).

Table 3992. Compounds of formula I.C, wherein R² is R²-15.602(=formula 1.3992).

Table 3993. Compounds of formula I.C, wherein R² is R²-15.603(=formula 1.3993).

Table 3994. Compounds of formula I.C, wherein R² is R²-15.604(=formula 1.3994).

Table 3995. Compounds of formula I.C, wherein R² is R²-15.605(=formula 1.3995).

Table 3996. Compounds of formula I.C, wherein R² is R²-15.606(=formula 1.3996).

Table 3997. Compounds of formula I.C, wherein R² is R²-15.607(=formula 1.3997).

Table 3998. Compounds of formula I.C, wherein R² is R²-15.608(=formula 1.3998).

Table 3999. Compounds of formula I.C, wherein R² is R²-15.609(=formula 1.3999).

Table 4000. Compounds of formula I.C, wherein R² is R²-15.610(=formula 1.4000).

Table 4001. Compounds of formula I.C, wherein R² is R²-15.611(=formula 1.4001).

Table 4002. Compounds of formula I.C, wherein R² is R²-15.612(=formula 1.4002).

Table 4003. Compounds of formula I.C, wherein R² is R²-15.613(=formula 1.4003).

Table 4004. Compounds of formula I.C, wherein R² is R²-15.614(=formula 1.4004).

Table 4005. Compounds of formula I.C, wherein R² is R²-15.615(=formula 1.4005).

Table 4006. Compounds of formula I.C, wherein R² is R²-15.616(=formula 1.4006).

Table 4007. Compounds of formula I.C, wherein R² is R²-15.617(=formula 1.4007).

Table 4008. Compounds of formula I.C, wherein R² is R²-15.618(=formula 1.4008).

Table 4009. Compounds of formula I.C, wherein R² is R²-15.619(=formula 1.4009).

Table 4010. Compounds of formula I.C, wherein R² is R²-15.620(=formula 1.4010).

Table 4011. Compounds of formula I.C, wherein R² is R²-15.621(=formula 1.4011).

Table 4012. Compounds of formula I.C, wherein R² is R²-15.622(=formula 1.4012).

Table 4013. Compounds of formula I.C, wherein R² is R²-15.623(=formula 1.4013).

Table 4014. Compounds of formula I.C, wherein R² is R²-15.624(=formula 1.4014).

Table 4015. Compounds of formula I.C, wherein R² is R²-15.625(=formula 1.4015).

Table 4016. Compounds of formula I.C, wherein R² is R²-15.626(=formula 1.4016).

Table 4017. Compounds of formula I.C, wherein R² is R²-15.627(=formula 1.4017).

Table 4018. Compounds of formula I.C, wherein R² is R²-15.628(=formula 1.4018).

Table 4019. Compounds of formula I.C, wherein R² is R²-15.629(=formula 1.4019).

Table 4020. Compounds of formula I.C, wherein R² is R²-15.630(=formula 1.4020).

Table 4021. Compounds of formula I.C, wherein R² is R²-15.631(=formula 1.4021).

Table 4022. Compounds of formula I.C, wherein R² is R²-15.632(=formula 1.4022).

Table 4023. Compounds of formula I.C, wherein R² is R²-15.633(=formula 1.4023).

Table 4024. Compounds of formula I.C, wherein R² is R²-15.634(=formula 1.4024).

Table 4025. Compounds of formula I.C, wherein R² is R²-15.635(=formula 1.4025).

Table 4026. Compounds of formula I.C, wherein R² is R²-15.636(=formula 1.4026).

Table 4027. Compounds of formula I.C, wherein R² is R²-15.637(=formula 1.4027).

Table 4028. Compounds of formula I.C, wherein R² is R²-15.638(=formula 1.4028).

Table 4029. Compounds of formula I.C, wherein R² is R²-15.639(=formula 1.4029).

Table 4030. Compounds of formula I.C, wherein R² is R²-15.640(=formula 1.4030).

Table 4031. Compounds of formula I.C, wherein R² is R²-15.641(=formula 1.4031).

Table 4032. Compounds of formula I.C, wherein R² is R²-15.642(=formula 1.4032).

Table 4033. Compounds of formula I.C, wherein R² is R²-15.643(=formula 1.4033).

Table 4034. Compounds of formula I.C, wherein R² is R²-15.644(=formula 1.4034).

Table 4035. Compounds of formula I.C, wherein R² is R²-15.645(=formula 1.4035).

Table 4036. Compounds of formula I.C, wherein R² is R²-15.646(=formula 1.4036).

Table 4037. Compounds of formula I.C, wherein R² is R²-15.647(=formula 1.4037).

Table 4038. Compounds of formula I.C, wherein R² is R²-15.648(=formula 1.4038).

Table 4039. Compounds of formula I.C, wherein R² is R²-15.649(=formula 1.4039).

Table 4040. Compounds of formula I.C, wherein R² is R²-15.650(=formula 1.4040).

Table 4041. Compounds of formula I.C, wherein R² is R²-15.651(=formula 1.4041).

Table 4042. Compounds of formula I.C, wherein R² is R²-15.652(=formula 1.4042).

Table 4043. Compounds of formula I.C, wherein R² is R²-15.653(=formula 1.4043).

Table 4044. Compounds of formula I.C, wherein R² is R²-15.654(=formula 1.4044).

Table 4045. Compounds of formula I.C, wherein R² is R²-15.655(=formula 1.4045).

Table 4046. Compounds of formula I.C, wherein R² is R²-15.656(=formula 1.4046).

Table 4047. Compounds of formula I.C, wherein R² is R²-15.657(=formula 1.4047).

Table 4048. Compounds of formula I.C, wherein R² is R²-15.658(=formula 1.4048).

Table 4049. Compounds of formula I.C, wherein R² is R²-15.659(=formula 1.4049).

Table 4050. Compounds of formula I.C, wherein R² is R²-15.660(=formula 1.4050).

Table 4051. Compounds of formula I.C, wherein R² is R²-15.661(=formula 1.4051).

Table 4052. Compounds of formula I.C, wherein R² is R²-15.662(=formula 1.4052).

Table 4053. Compounds of formula I.C, wherein R² is R²-15.663(=formula 1.4053).

Table 4054. Compounds of formula I.C, wherein R² is R²-15.664(=formula 1.4054).

Table 4055. Compounds of formula I.C, wherein R² is R²-15.665(=formula 1.4055).

Table 4056. Compounds of formula I.C, wherein R² is R²-15.666(=formula 1.4056).

Table 4057. Compounds of formula I.C, wherein R² is R²-15.667(=formula 1.4057).

Table 4058. Compounds of formula I.C, wherein R² is R²-15.668(=formula 1.4058).

Table 4059. Compounds of formula I.C, wherein R² is R²-15.669(=formula 1.4059).

Table 4060. Compounds of formula I.C, wherein R² is R²-15.670(=formula 1.4060).

Table 4061. Compounds of formula I.C, wherein R² is R²-15.671(=formula 1.4061).

Table 4062. Compounds of formula I.C, wherein R² is R²-15.672(=formula 1.4062).

Table 4063. Compounds of formula I.C, wherein R² is R²-15.673(=formula 1.4063).

Table 4064. Compounds of formula I.C, wherein R² is R²-15.674(=formula 1.4064).

Table 4065. Compounds of formula I.C, wherein R² is R²-15.675(=formula 1.4065).

Table 4066. Compounds of formula I.C, wherein R² is R²-15.676(=formula 1.4066).

Table 4067. Compounds of formula I.C, wherein R² is R²-9.1(=formula 1.4067).

Table 4068. Compounds of formula I.C, wherein R² is R²-9.2(=formula 1.4068).

Table 4069. Compounds of formula I.C, wherein R² is R²-9.3(=formula 1.4069).

Table 4070. Compounds of formula I.C, wherein R² is R²-9.4(=formula 1.4070).

Table 4071. Compounds of formula I.C, wherein R² is R²-9.5(=formula 1.4071).

Table 4072. Compounds of formula I.C, wherein R² is R²-9.6(=formula 1.4072).

Table 4073. Compounds of formula I.C, wherein R² is R²-9.7(=formula 1.4073).

Table 4074. Compounds of formula I.C, wherein R² is R²-9.8(=formula 1.4074).

Table 4075. Compounds of formula I.C, wherein R² is R²-9.9(=formula 1.4075).

Table 4076. Compounds of formula I.C, wherein R² is R²-9.10(=formula 1.4076).

Table 4077. Compounds of formula I.C, wherein R² is R²-9.11(=formula 1.4077).

Table 4078. Compounds of formula I.C, wherein R² is R²-9.12(=formula 1.4078).

Table 4079. Compounds of formula I.C, wherein R² is R²-9.13(=formula 1.4079).

Table 4080. Compounds of formula I.C, wherein R² is R²-9.14(=formula 1.4080).

Table 4081. Compounds of formula I.C, wherein R² is R²-9.15(=formula 1.4081).

Table 4082. Compounds of formula I.C, wherein R² is R²-9.16(=formula 1.4082).

TABLE A2

| Line | R¹ | R³ | m | R⁴ |
|---|---|---|---|---|
| I-337 | c-C₃H₅ | CH₃ | 0 | — |
| I-338 | c-C₃H₅ | OCH₃ | 0 | — |
| I-339 | c-C₃H₅ | Cl | 0 | — |
| I-340 | c-C₃H₅ | Br | 0 | — |
| I-341 | c-C₃H₅ | CHF₂ | 0 | — |
| I-342 | c-C₃H₅ | F | 0 | — |
| I-343 | c-C₃H₅ | I | 0 | — |
| I-344 | c-C₃H₅ | CH₃ | 1 | 2″-F |
| I-345 | c-C₃H₅ | OCH₃ | 1 | 2″-F |
| I-346 | c-C₃H₅ | Cl | 1 | 2″-F |
| I-347 | c-C₃H₅ | Br | 1 | 2″-F |
| I-348 | c-C₃H₅ | CHF₂ | 1 | 2″-F |
| I-349 | c-C₃H₅ | F | 1 | 2″-F |
| I-350 | c-C₃H₅ | I | 1 | 2″-F |
| I-351 | c-C₄H₇ | CH₃ | 0 | — |
| I-352 | c-C₄H₇ | OCH₃ | 0 | — |
| I-353 | c-C₄H₇ | Cl | 0 | — |
| I-354 | c-C₄H₇ | Br | 0 | — |
| I-355 | c-C₄H₇ | CHF₂ | 0 | — |
| I-356 | c-C₄H₇ | F | 0 | — |
| I-357 | c-C₄H₇ | I | 0 | — |
| I-358 | c-C₄H₇ | CH₃ | 1 | 2″-F |
| I-359 | c-C₄H₇ | OCH₃ | 1 | 2″-F |
| I-360 | c-C₄H₇ | Cl | 1 | 2″-F |
| I-361 | c-C₄H₇ | Br | 1 | 2″-F |
| I-362 | c-C₄H₇ | CHF₂ | 1 | 2″-F |
| I-363 | c-C₄H₇ | F | 1 | 2″-F |
| I-364 | c-C₄H₇ | I | 1 | 2″-F |
| I-365 | C₂H₅ | CH₃ | 0 | — |
| I-366 | C₂H₅ | OCH₃ | 0 | — |
| I-367 | C₂H₅ | Cl | 0 | — |
| I-368 | C₂H₅ | Br | 0 | — |
| I-369 | C₂H₅ | CHF₂ | 0 | — |
| I-370 | C₂H₅ | F | 0 | — |
| I-371 | C₂H₅ | I | 0 | — |
| I-372 | C₂H₅ | CH₃ | 1 | 2″-F |
| I-373 | C₂H₅ | OCH₃ | 1 | 2″-F |
| I-374 | C₂H₅ | Cl | 1 | 2″-F |
| I-375 | C₂H₅ | Br | 1 | 2″-F |
| I-376 | C₂H₅ | CHF₂ | 1 | 2″-F |
| I-377 | C₂H₅ | F | 1 | 2″-F |
| I-378 | C₂H₅ | I | 1 | 2″-F |
| I-379 | OCH₃ | CH₃ | 0 | — |
| I-380 | OCH₃ | OCH₃ | 0 | — |
| I-381 | OCH₃ | Cl | 0 | — |
| I-382 | OCH₃ | Br | 0 | — |
| I-383 | OCH₃ | CHF₂ | 0 | — |
| I-384 | OCH₃ | F | 0 | — |
| I-385 | OCH₃ | I | 0 | — |
| I-386 | OCH₃ | CH₃ | 1 | 2″-F |
| I-387 | OCH₃ | OCH₃ | 1 | 2″-F |
| I-388 | OCH₃ | Cl | 1 | 2″-F |
| I-389 | OCH₃ | Br | 1 | 2″-F |
| I-390 | OCH₃ | CHF₂ | 1 | 2″-F |
| I-391 | OCH₃ | F | 1 | 2″-F |
| I-392 | OCH₃ | I | 1 | 2″-F |

The specific number for each single compound is deductible as follows:

Compound 1.2055.I-392 e.g. comprises the compound of formula 1.2055 from Table 2055 and line I-392 from Table A2;

To widen the spectrum of action and to achieve synergistic effects, the pyrimidine compounds of formula (I) may be mixed with a large number of representatives of other herbicidal or growth-regulating active ingredient groups and then applied concomitantly. Suitable components for mixtures are, e.g., herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, or ureas.

It may furthermore be beneficial to apply the pyrimidine compounds of formula (I) alone or in combination with other herbicides, or else in the form of a mixture with other crop protection agents, e.g. together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

In one embodiment of the present invention the compositions according to the present invention comprise at least one pyrimidine compound of formula (I) (compound A) and at least one further active compound selected from herbicides B, preferably herbicides B of class b1) to b15), and safeners C (compound C).

In a preferred embodiment of the invention, the composition comprises as active compound A or component A at least one, preferably exactly one, pyrimidine compound of formula (I.1) (corresponds to pyrimidine compound of formula (I)), as defined herein;

In another preferred embodiment of the invention, the composition comprises as active compound A or component A at least one, preferably exactly one, pyrimidine compound of formula (I.2) (corresponds to pyrimidine compound of formula (I)), as defined herein;

In another preferred embodiment of the invention, the composition comprises as active compound A or component A at least one, preferably exactly one, pyrimidine compound of formula (I.3) (corresponds to pyrimidine compound of formula (I)), as defined herein;

In another preferred embodiment of the invention, the composition comprises as active compound A or component A at least one, preferably exactly one, pyrimidine compound of formula (I.4) (corresponds to pyrimidine compound of formula (I)), as defined herein;

In another preferred embodiment of the invention, the composition comprises as active compound A or component A at least one, preferably exactly one, pyrimidine compound of formula (I.5) (corresponds to pyrimidine compound of formula (I)), as defined herein;

In another preferred embodiment of the invention, the composition comprises as active compound A or component A at least one, preferably exactly one, pyrimidine compound of formula (I.6) (corresponds to pyrimidine compound of formula (I)), as defined herein;

In another preferred embodiment of the invention, the composition comprises as active compound A or component A at least one, preferably exactly one, pyrimidine compound of formula (I.7) (corresponds to pyrimidine compound of formula (I)), as defined herein;

In another preferred embodiment of the invention, the composition comprises as active compound A or component A at least one, preferably exactly one, pyrimidine compound of formula (I.8) (corresponds to pyrimidine compound of formula (I)), as defined herein;

In another preferred embodiment of the invention, the composition comprises as active compound A or component A at least one, preferably exactly one, pyrimidine compound of formula (I.9) (corresponds to pyrimidine compound of formula (I)), as defined herein;

In another preferred embodiment of the invention, the composition comprises as active compound A or component A at least one, preferably exactly one, pyrimidine compound of formula (I.10) (corresponds to pyrimidine compound of formula (I)), as defined herein;

Preferred compounds of the formula (I) which, as component A, are constituent of the composition according to the invention are the compounds 1.1 to 1.10, as defined above;

In another embodiment of the present invention the compositions according to the present invention comprise at least one pyrimidine compound of formula (I) and at least one further active compound B (herbicide B).

The further herbicidal compound B (component B) is preferably selected from the herbicides of class b1) to b15):

Mixing partners for the composition can be selected from below herbicides B as defined below:

B) herbicides of class b1) to b15):

b1) lipid biosynthesis inhibitors;

b2) acetolactate synthase inhibitors (ALS inhibitors);

b3) photosynthesis inhibitors;

b4) protoporphyrinogen-IX oxidase inhibitors (PPO inhibitors);

b5) bleacher herbicides;

b6) enolpyruvyl shikimate 3-phosphate synthase inhibitors (EPSP inhibitors);

b7) glutamine synthetase inhibitors;

b8) 7,8-dihydropteroate synthase inhibitors (DHP inhibitors);

b9) mitosis inhibitors;

b10) inhibitors of the synthesis of very long chain fatty acids (VLCFA inhibitors);

b11) cellulose biosynthesis inhibitors;

b12) decoupler herbicides;

b13) auxinic herbicides;

b14) auxin transport inhibitors; and

b15) other herbicides selected from the group consisting of bromobutide, chlorflurenol, chlorflurenol-methyl, cinmethylin, cumyluron, dalapon, dazomet, difenzoquat, difenzoquatmetilsulfate, dimethipin, DSMA, dymron, endothal and its salts, etobenzanid, flamprop, flampropisopropyl, flamprop-methyl, flamprop-M-isopropyl, flamprop-M-methyl, flurenol, flurenolbutyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters;

including their agriculturally acceptable salts or derivatives;

In one embodiment of the invention, the compositions contain at least one inhibitor of the lipid biosynthesis (herbicide b1). These compounds inhibit lipid biosynthesis. Inhibition of the lipid biosynthesis can be affected either through inhibition of acetylCoA carboxylase (hereinafter-termed ACCase herbicides) or through a different mode of action (hereinafter termed non-AC-Case herbicides). The ACCase herbicides belong to the group A of the HRAC classification system whereas the non-ACCase herbicides belong to the group N of the HRAC classification.

In another embodiment of the invention, the compositions contain at least one ALS inhibitor (herbicide b2). The herbicidal activity of these compounds is based on the inhibition of acetolactate synthase and thus on the inhibition of the branched chain amino acid biosynthesis. These inhibitors belong to the group B of the HRAC classification system.

In another embodiment of the invention, the compositions contain at least one inhibitor of photosynthesis (herbicide b3). The herbicidal activity of these compounds is based either on the inhibition of the photosystem II in plants (so-called PSII inhibitors, groups C₁, C₂ and C₃ of HRAC classification) or on diverting the electron transfer in photosystem I in plants (so-called PSI inhibitors, group D of HRAC classification) and thus on an inhibition of photosynthesis. Amongst these, PSII inhibitors are preferred.

In another embodiment of the invention, the compositions contain at least one inhibitor of protoporphyrinogen-IX-oxidase (herbicide b4). The herbicidal activity of these compounds is based on the inhibition of the protoporphyrinogen-IX-oxidase. These inhibitors belong to the group E of the HRAC classification system.

In another embodiment of the invention, the compositions contain at least one bleacher-herbicide (herbicide b5). The herbicidal activity of these compounds is based on the inhibition of the carotenoid biosynthesis. These include compounds which inhibit carotenoid biosynthesis by inhibition of phytoene desaturase (so-called PDS inhibitors, group F1 of HRAC classification), compounds that inhibit the 4-hydroxyphenylpyruvate-dioxygenase (HPPD inhibitors, group F2 of HRAC classification), compounds that inhibit DOXsynthase (group F4 of HRAC class) and compounds which inhibit carotenoid biosynthesis by an unknown mode of action (bleacher—unknown target, group F3 of HRAC classification).

In another embodiment of the invention, the compositions contain at least one EPSP synthase inhibitor (herbicide b6). The herbicidal activity of these compounds is based on the inhibition of enolpyruvyl shikimate 3-phosphate synthase, and thus on the inhibition of the amino acid biosynthesis in plants. These inhibitors belong to the group G of the HRAC classification system.

In another embodiment of the invention, the compositions contain at least one glutamine synthetase inhibitor (herbicide b7). The herbicidal activity of these compounds is based on the inhibition of glutamine synthetase, and thus on the inhibition of the aminoacid biosynthesis in plants. These inhibitors belong to the group H of the HRAC classification system.

In another embodiment of the invention, the compositions contain at least one DHP synthase inhibitor (herbicide b8). The herbicidal activity of these compounds is based on the inhibition of 7,8-dihydropteroate synthase. These inhibitors belong to the group I of the HRAC classification system.

In another embodiment of the invention, the compositions contain at least one mitosis inhibitor (herbicide b9). The herbicidal activity of these compounds is based on the disturbance or inhibition of microtubule formation or organization, and thus on the inhibition of mitosis. These inhibitors belong to the groups K1 and K2 of the HRAC classification system. Among these, compounds of the group K1, in particular dinitroanilines, are preferred.

In another embodiment of the invention, the compositions contain at least one VLCFA inhibitor (herbicide b10). The herbicidal activity of these compounds is based on the inhibition of the synthesis of very long chain fatty acids and thus on the disturbance or inhibition of cell division in plants. These inhibitors belong to the group K3 of the HRAC classification system.

In another embodiment of the invention, the compositions contain at least one cellulose biosynthesis inhibitor (herbicide b11). The herbicidal activity of these compounds is based on the inhibition of the biosynthesis of cellulose and thus on the inhibition of the synthesis of cell walls in plants. These inhibitors belong to the group L of the HRAC classification system.

In another embodiment of the invention, the compositions contain at least one decoupler herbicide (herbicide b12). The herbicidal activity of these compounds is based on the disruption of the cell membrane. These inhibitors belong to the group M of the HRAC classification system.

In another embodiment of the invention, the compositions contain at least one auxinic herbicide (herbicide b13). These include compounds that mimic auxins, i.e. plant hormones, and affect the growth of the plants. These compounds belong to the group O of the H RAC classification system.

In another embodiment of the invention, the compositions contain at least one auxin transport inhibitor (herbicide b14). The herbicidal activity of these compounds is based on the inhibition of the auxin transport in plants. These compounds belong to the group P of the HRAC classification system.

As to the given mechanisms of action and classification of the active substances, see e.g. “HRAC, Classification of Herbicides According to Mode of Action”, http://www.plantprotection.org/hrac/MOA.html).

Preference is given to those compositions according to the present invention comprising at least one herbicide B selected from herbicides of class b1, b2, b3, b4, b5, b6, b9, b10, b13, and b14.

Specific preference is given to those compositions according to the present invention which comprise at least one herbicide B selected from the herbicides of class b1, b2, b4, b5, b9, b10, b13, and b14.

Particular preference is given to those compositions according to the present invention which comprise at least one herbicide B selected from the herbicides of class b1, b2, b4, b5, b9, b10, and b13

Examples of herbicides B which can be used in combination with the compound of formula (I) according to the present invention are:

b1) from the group of the lipid biosynthesis inhibitors:

ACC-herbicides such as alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim,

4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); and non ACC herbicides such as benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;

b2) from the group of the ALS inhibitors:

sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl and tritosulfuron,

imidazolinones such as imazamethabenz, imazamethabenz-methyl, imazamox, imazapic, imazapyr, imazaquin and imazethapyr, triazolopyrimidine herbicides and sulfonanilides such as cloransulam, cloransulam-methyl, diclosulam, flumetsulam, florasulam, metosulam, penoxsulam, pyrimisulfan and pyroxsulam,

pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl) oxy]phenyl]methyl]amino]-benzoic acid-1-methylethyl ester (CAS 420138-41-6), 4-[[[2-[(4,6-dimethoxy-2-pyrimidinyl)oxy]phenyl]methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8),

sulfonylaminocarbonyl-triazolinone herbicides such as flucarbazone, flucarbazone-sodium, propoxycarbazone, propoxycarbazone-sodium, thiencarbazone and thiencarbazone-methyl; and triafamone:

among these, a preferred embodiment of the invention relates to those compositions comprising at least one imidazolinone herbicide;

b3) from the group of the photosynthesis inhibitors:

amicarbazone, inhibitors of the photosystem II, e.g. 1-(6-tert-butylpyrimidin-4-yl)-2-hydroxy-4-methoxy-3-methyl-2H-pyrrol-5-one (CAS 1654744-66-7), 1-(5-tert-butylisoxazol-3-yl)-2-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butyl-1-methyl-pyrazol-3-yl)-4-chloro-2-hydroxy-3-methyl-2H-pyrrol-5-one (CAS 1654057-29-0), 1-(5-tert-butyl-1-methyl-pyrazol-3-yl)-3-chloro-2-hydroxy-4-methyl-2H-pyrrol-5-one (CAS 1654747-80-4), 4-hydroxy-1-methoxy-5-methyl-3-[4-(trifluoromethyl)-2-pyridyl]imidazolidin-2-one; (CAS 2023785-78-4), 4-hydroxy-1,5-dimethyl-3-[4-(trifluoromethyl)-2-pyridyl]imidazolidin-2-one (CAS 2023785-79-5), 5-ethoxy-4-hydroxy-1-methyl-3-[4-(trifluoromethyl)-2-pyridyl]imidazolidin-2-one (CAS 1701416-69-4), 4-hydroxy-1-methyl-3-[4-(trifluoromethyl)-2-pyridyl]imidazolidin-2-one (CAS 1708087-22-2), 4-hydroxy-1,5-dimethyl-3-[1-methyl-5-(trifluoromethyl)pyrazol-3-yl]imidazolidin-2-one (CAS 2023785-80-8), 1-(5-tert-butylisoxazol-3-yl)-4-ethoxy-5-hydroxy-3-methyl-imidazolidin-2-one (CAS 1844836-64-1), triazine herbicides, including of chlorotriazine, triazinones, triazindiones, methylthiotriazines and pyridazinones such as ametryn, atrazine, chloridazone, cyanazine, desmetryn, dimethametryn, hexazinone, metribuzin, prometon, prometryn, propazine, simazine, simetryn, terbumeton, terbuthylazin, terbutryn and trietazin, aryl urea such as chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron and thiadiazuron, phenyl carbamates such as desmedipham, karbutilat, phenmedipham, phenmedipham-ethyl, nitrile herbicides such as bromofenoxim, bromoxynil and its salts and esters, ioxynil and its salts and esters, uraciles such as bromacil, lenacil and terbacil, and bentazon and bentazon-sodium, pyridate, pyridafol, pentanochlor and propanil and inhibitors of the photosystem I such as diquat, diquat-dibromide, paraquat, paraquat-dichloride and paraquat-dimetilsulfate. Among these, a preferred embodiment of the invention relates to those compositions comprising at least one aryl urea herbicide. Among these, likewise a preferred embodiment of the invention relates to those compositions comprising at least one triazine herbicide. Among these, likewise a preferred embodiment of the invention relates to those compositions comprising at least one nitrile herbicide;

b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chlomethoxyfen, chlorphthalim, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoro-methyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), methyl (E)-4-[2-chloro-5-[4-chloro-5-(difluoromethoxy)-1H-methyl-pyrazol-3-yl]-4-fluoro-phenoxy]-3-methoxy-but-2-enoate (CAS 948893-00-3), and 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4);

b5) from the group of the bleacher herbicides:

PDS inhibitors: beflubutamid, diflufenican, fluridone, flurochloridone, flurtamone, norflurazon, picolinafen, and 4-(3-trifluoromethylphenoxy)-2-(4-trifluoromethylphenyl)pyrimidine (CAS 180608-33-7), HPPD inhibitors: benzobicyclon, benzofenap, bicyclopyrone, clomazone, fenquinotrione, isoxaflutole, mesotrione, oxotrione (CAS 1486617-21-3), pyrasulfotole, pyrazolynate, pyrazoxyfen, sulcotrione, tefuryltrione, tembotrione, tolpyralate, topramezone, bleacher, unknown target: aclonifen, amitrole flumeturon, 2-chloro-3-methylsulfanyl-N-(1-methyltetrazol-5-yl)-4-(trifluoromethyl)benzamide (CAS 1361139-71-0), 2-(2,4-dichlorophenyl)methyl-4,4-dimethyl-3-isoxazolidone (CAS 81777-95-9) and 2-(2,5-dichlorophenyl)methyl-4,4-dimethyl-3-isoxazolidinone (CAS 81778-66-7);

b6) from the group of the EPSP synthase inhibitors: glyphosate, glyphosate-isopropylammonium, glyposate-potassium and glyphosate-trimesium (sulfosate);

b7) from the group of the glutamine synthase inhibitors: bilanaphos (bialaphos), bilanaphossodium, glufosinate, glufosinate-P and glufosinate-ammonium;

b8) from the group of the DHP synthase inhibitors: asulam;

b9) from the group of the mitosis inhibitors:

compounds of group K1: dinitroanilines such as benfluralin, butralin, dinitramine, ethalfluralin, fluchloralin, oryzalin, pendimethalin, prodiamine and trifluralin, phosphoramidates such as amiprophos, amiprophos-methyl, and butamiphos, benzoic acid herbicides such as chlorthal, chlorthal-dimethyl, pyridines such as dithiopyr and thiazopyr, benzamides such as propyzamide and tebutam; compounds of group K2: carbetamide, chlorpropham, flamprop, flamprop-isopropyl, flamprop-methyl, flamprop-M-isopropyl, flamprop-M-methyl and propham; among these, compounds of group K1, in particular dinitroanilines are preferred;

b10) from the group of the VLCFA inhibitors:

chloroacetamides such as acetochlor, alachlor, amidochlor, butachlor, dimethachlor, dimethenamid, dimethenamid-P, metazachlor, metolachlor, metolachlor-S, pethoxamid, pretilachlor, propachlor, propisochlor and thenylchlor, oxyacetanilides such as flufenacet and mefenacet, acetanilides such as diphenamid, naproanilide, napropamide and napropamide-M, tetrazolinones such fentrazamide, and other herbicides such as anilofos, cafenstrole, fenoxasulfone, ipfencarbazone, piperophos, pyroxasulfone and isoxazoline compounds of the formulae II.1, II.2, II.3, II.4, II.5, II.6, II.7, II.8 and II.9

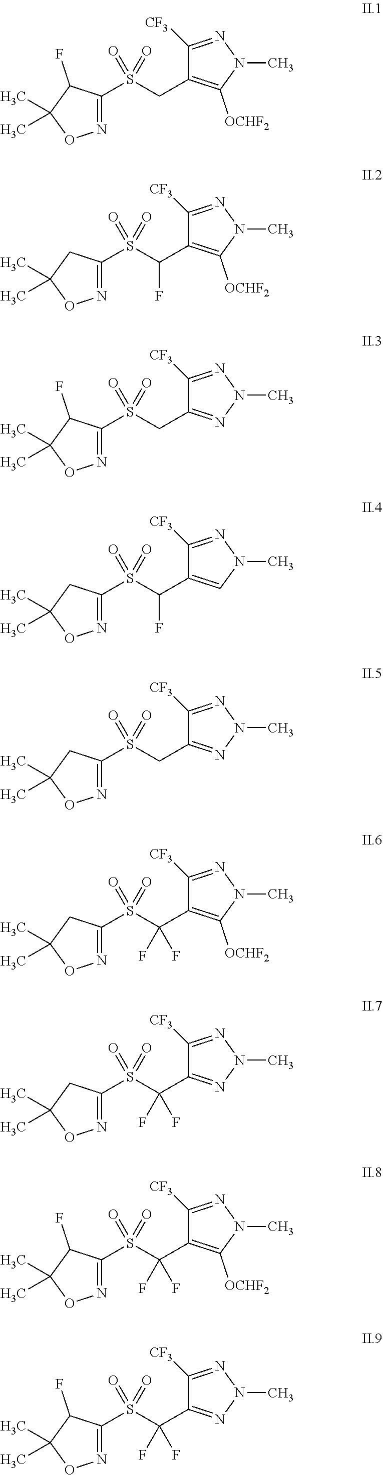

the isoxazoline compounds are known in the art, e.g. from WO 2006/024820, WO 2006/037945, WO 2007/071900 and WO 2007/096576;

among the VLCFA inhibitors, preference is given to chloroacetamides and oxyacetamides; b11) from the group of the cellulose biosynthesis inhibitors: chlorthiamid, dichlobenil, flupoxam, indaziflam, isoxaben, triaziflam and 1-cyclohexyl-5-pentafluorphenyloxy-14-[1,2,4,6]thiatriazin-3-ylamine (CAS 175899-01-1);

b12) from the group of the decoupler herbicides: dinoseb, dinoterb and DNOC and its salts;

b13) from the group of the auxinic herbicides:

2,4-D and its salts and esters such as clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, flopyrauxifen, fluroxypyr, fluroxypyr-butometyl, fluroxypyr-meptyl, halauxifen and its salts and esters (CAS 943832-60-8); MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);

b14) from the group of the auxin transport inhibitors: diflufenzopyr, diflufenzopyr-sodium, naptalam and naptalam-sodium;

b15) from the group of the other herbicides: bromobutide, chlorflurenol, chlorflurenol-methyl, cinmethylin, cumyluron, cyclopyrimorate (CAS 499223-49-3 and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine and tridiphane.

Preferred herbicides B that can be used in combination with the pyrimidine compounds of the formula (I) according to the present invention are:

b1) from the group of the lipid biosynthesis inhibitors:

clethodim, clodinafop-propargyl, cycloxydim, cyhalofop-butyl, diclofop-methyl, fenoxaprop-P-ethyl, fluazifop-P-butyl, haloxyfop-P-methyl, metamifop, pinoxaden, profoxydim, propaquizafop, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); benfuresate, dimepiperate, EPTC, esprocarb, ethofumesate, molinate, orbencarb, prosulfocarb, thiobencarb and triallate;

b2) from the group of the ALS inhibitors:

amidosulfuron, azimsulfuron, bensulfuron-methyl, bispyribac-sodium, chlorimuron-ethyl, chlorsulfuron, cloransulam-methyl, cyclosulfamuron, diclosulam, ethametsulfuron-methyl, ethoxysulfuron, flazasulfuron, florasulam, flucarbazone-sodium, flucetosulfuron, flumetsulam, flupyrsulfuron-methyl-sodium, foramsulfuron, halosulfuron-methyl, imazamethabenz-methyl, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, metosulam, metsulfuron-methyl, nicosulfuron, orthosulfamuron, oxasulfuron, penoxsulam, primisulfuron-methyl, propoxycarbazon-sodium, propyrisulfuron, prosulfuron, pyrazosulfuron-ethyl, pyribenzoxim, pyrimisulfan, pyriftalid, pyriminobac-methyl, pyrithiobac-sodium, pyroxsulam, rimsulfuron, sulfometuron-methyl, sulfosulfuron, thiencarbazone-methyl, thifensulfuron-methyl, triasulfuron, tribenuron-methyl, trifloxysulfuron, triflusulfuron-methyl, tritosulfuron and triafamone;

b3) from the group of the photosynthesis inhibitors:

ametryn, amicarbazone, atrazine, bentazone, bentazone-sodium, bromoxynil and its salts and esters, chloridazone, chlorotoluron, cyanazine, desmedipham, diquat-dibromide, diuron, fluometuron, hexazinone, ioxynil and its salts and esters, isoproturon, lenacil, linuron, metamitron, methabenzthiazuron, metribuzin, paraquat, paraquat-dichloride, phenmedipham, propanil, pyridate, simazine, terbutryn, terbuthylazine, thidiazuron, 1-(6-tert-butylpyrimidin-4-yl)-2-hydroxy-4-methoxy-3-methyl-2H-pyrrol-5-one (CAS 1654744-66-7), 1-(5-tert-butylisoxazol-3-yl)-2-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butyl-1-methyl-pyrazol-3-yl)-4-chloro-2-hydroxy-3-methyl-2H-pyrrol-5-one (CAS 1654057-29-0), 1-(5-tert-butyl-1-methyl-pyrazol-3-yl)-3-chloro-2-hydroxy-4-methyl-2H-pyrrol-5-one (CAS 1654747-80-4), 4-hydroxy-1-methoxy-5-methyl-3-[4-(trifluoromethyl)-2-pyridyl]imidazolidin-2-one; (CAS 2023785-78-4), 4-hydroxy-1,5-dimethyl-3-[4-(trifluoromethyl)-2-pyridyl]imidazolidin-2-one (CAS 2023785-79-5), 5-ethoxy-4-hydroxy-1-methyl-3-[4-(trifluoromethyl)-2-pyridyl]imidazolidin-2-one (CAS 1701416-69-4), 4-hydroxy-1-methyl-3-[4-(trifluoromethyl)-2-pyridyl]imidazolidin-2-one (CAS 1708087-22-2), 4-hydroxy-1,5-dimethyl-3-[1-methyl-5-(trifluoromethyl)pyrazol-3-yl]imidazolidin-2-one (CAS 2023785-80-8) and 1-(5-tert-butylisoxazol-3-yl)-4-ethoxy-5-hydroxy-3-methyl-imidazolidin-2-one (CAS 1844836-64-1);

b4) from the group of the protoporphyrinogen-IX oxidase inhibitors:

acifluorfen-sodium, bencarbazone, benzfendizone, butafenacil, carfentrazone-ethyl, cinidon-ethyl, flufenpyr-ethyl, flumiclorac-pentyl, flumioxazin, fluoroglycofen-ethyl, fomesafen, lactofen, oxadiargyl, oxadiazon, oxyfluorfen, pentoxazone, pyraflufen, pyraflufen-ethyl, saflufenacil, sulfentrazone, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0); 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), and 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4);

b5) from the group of the bleacher herbicides:

aclonifen, amitrole, beflubutamid, benzobicyclon, bicyclopyrone, clomazone, diflufenican, fenquinotrione, flumeturon, flurochloridone, flurtamone, isoxaflutole, mesotrione, oxotrione (CAS 1486617-21-3), norflurazon, picolinafen, pyrasulfotole, pyrazolynate, sulcotrione, tefuryltrione, tembotrione, tolpyralate, topramezone, 4-(3-trifluoromethylphenoxy)-2-(4-trifluoromethylphenyl)-pyrimidine (CAS 180608-33-7), 2-chloro-3-methylsulfanyl-N-(1-methyltetrazol-5-yl)-4-(trifluoro-methyl)benzamide (CAS 1361139-71-0, 2-(2,4-dichlorophenyl)methyl-4,4-dimethyl-3-isoxazolidone (CAS 81777-95-9) and 2-(2,5-dichlorophenyl)methyl-4,4-dimethyl-3-isoxazolidinone (CAS 81778-66-7);

b6) from the group of the EPSP synthase inhibitors:

glyphosate, glyphosate-isopropylammonium, glyphosate-potassium and glyphosate-trimesium (sulfosate);

b7) from the group of the glutamine synthase inhibitors:

glufosinate, glufosinate-P, glufosinate-ammonium;

b8) from the group of the DHP synthase inhibitors: asulam;

b9) from the group of the mitosis inhibitors:

benfluralin, dithiopyr, ethalfluralin, flamprop, flamprop-isopropyl, flamprop-methyl, flamprop-M-isopropyl, flamprop-M-methyl, oryzalin, pendimethalin, thiazopyr and trifluralin;

b10) from the group of the VLCFA inhibitors: acetochlor, alachlor, amidochlor, anilofos, butachlor, cafenstrole, dimethenamid, dimethenamid-P, fentrazamide, flufenacet, mefenacet, metazachlor, metolachlor, S-metolachlor, naproanilide, napropamide, napropamide-M, pretilachlor, fenoxasulfone, ipfencarbazone, pyroxasulfone thenylchlor and isoxazoline-compounds of the formulae II.1, II.2, II.3, II.4, II.5, II.6, II.7, II.8 and II.9 as mentioned above; b11) from the group of the cellulose biosynthesis inhibitors: dichlobenil, flupoxam, indaziflam, isoxaben, triaziflam and 1-cyclohexyl-5-pentafluorphenyloxy-14-[1,2,4,6]thiatriazin-3-ylamine (CAS 175899-01-1);

b13) from the group of the auxinic herbicides:

2,4-D and its salts and esters, aminocyclopyrachlor and its salts and esters, aminopyralid and its salts such as aminopyralid-dimethylammonium, aminopyralid-tris(2-hydroxypropyl)ammonium and its esters, clopyralid and its salts and esters, dicamba and its salts and esters, dichlor-prop-P and its salts and esters, flopyrauxifen, fluroxypyr-meptyl, halauxifen and its salts and esters (CAS 943832-60-8), MCPA and its salts and esters, MCPB and its salts and esters, mecoprop-P and its salts and esters, picloram and its salts and esters, quinclorac, quinmerac, triclopyr and its salts and esters, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);

b14) from the group of the auxin transport inhibitors: diflufenzopyr and diflufenzopyr-sodium;

b15) from the group of the other herbicides: bromobutide, cinmethylin, cumyluron, cyclopyrimorate (CAS 499223-49-3) and its salts and esters, dalapon, difenzoquat, difenzoquat-metilsulfate, DSMA, dymron (=daimuron), indanofan, metam, methylbromide, MSMA, oxaziclomefone, pyributicarb and tridiphane.

Particularly preferred herbicides B that can be used in combination with the pyrimidine compounds of the formula (I) according to the present invention are:

b1) from the group of the lipid biosynthesis inhibitors: clodinafop-propargyl, cycloxydim, cyhalofop-butyl, fenoxaprop-P-ethyl, pinoxaden, profoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetra-methyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetra-methyl-2H-pyran-3-one (CAS 1312337-48-6); 5-(Acetyloxy)-4-(2′,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2′,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); esprocarb, prosulfocarb, thiobencarb and triallate;

b2) from the group of the ALS inhibitors: bensulfuron-methyl, bispyribac-sodium, cyclosulfamuron, diclosulam, flumetsulam, flupyrsulfuron-methyl-sodium, foramsulfuron, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, nicosulfuron, penoxsulam, propoxycarbazon-sodium, propyrisulfuron, pyrazosulfuron-ethyl, pyroxsulam, rimsulfuron, sulfosulfuron, thiencarbazon-methyl, tritosulfuron and triafamone;

b3) from the group of the photosynthesis inhibitors: ametryn, atrazine, diuron, fluometuron, hexazinone, isoproturon, linuron, metribuzin, paraquat, paraquat-dichloride, propanil, terbutryn, terbuthylazine, 1-(5-tert-butylisoxazol-3-yl)-2-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butylisoxazol-3-yl)-4-ethoxy-5-hydroxy-3-methyl-imidazolidin-2-one (CAS 1844836-64-1);

b4) from the group of the protoporphyrinogen-IX oxidase inhibitors: flumioxazin, oxyfluorfen, pyraflufen, pyraflufen-ethyl, saflufenacil, sulfentrazone, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100, 3-[7-fluoro-3-oxo-4-(prop-2-ynyl)-3,4-dihydro-2H-ben-[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), and 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0);

b5) from the group of the bleacher herbicides: amitrole, bicyclopyrone, clomazone, diflufenican, fenquinotrione, flumeturon, flurochloridone, isoxaflutole, mesotrione, oxotrione (CAS 1486617-21-3), picolinafen, sulcotrione, tefuryltrione, tembotrione, tolpyralate, topramezone, 2-chloro-3-methylsulfanyl-N-(1-methyltetrazol-5-yl)-4-(trifluoromethyl)benzamide (CAS 1361139-71-0), 2-(2,4-dichlorophenyl)methyl-4,4-dimethyl-3-isoxazolidone (CAS 81777-95-9); and 2-(2,5-dichlorophenyl)methyl-4,4-dimethyl-3-isoxazolidinone (CAS 81778-66-7);

b6) from the group of the EPSP synthase inhibitors: glyphosate, glyphosate-isopropylammonium and glyphosate-trimesium (sulfosate);

b7) from the group of the glutamine synthase inhibitors: glufosinate, glufosinate-P and glufosinate-ammonium;

b9) from the group of the mitosis inhibitors: pendimethalin and trifluralin;

b10) from the group of the VLCFA inhibitors: acetochlor, cafenstrole, dimethenamid-P, fentrazamide, flufenacet, mefenacet, metazachlor, metolachlor, S-metolachlor, fenoxasulfone, ipfencarbazone and pyroxasulfone; likewise, preference is given to isoxazoline compounds of the formulae II.1, II.2, II.3, II.4, II.5, II.6, II.7, II.8 and II.9 as mentioned above;

b11) from the group of the cellulose biosynthesis inhibitors: indaziflam, isoxaben and triaziflam;

b13) from the group of the auxinic herbicides: 2,4-D and its salts and esters such as clacyfos, and aminocyclopyrachlor and its salts and esters, aminopyralid and its salts and its esters, clopyralid and its salts and esters, dicamba and its salts and esters, flopyrauxifen, fluroxypyrmeptyl, halauxifen, halauxifen-methyl, quinclorac, quinmerac, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);

b14) from the group of the auxin transport inhibitors: diflufenzopyr and diflufenzopyr-sodium, b15) from the group of the other herbicides: cinmethylin, dymon (=daimuron), indanofan, oxaziclomefone.

Particularly preferred herbicides B are the herbicides B as defined above; in particular, the herbicides B.1-B.202 listed below in table B:

TABLE B

| B | Herbicide B |
|---|---|
| B.1 | clethodim |
| B.2 | clodinafop-propargyl |
| B.3 | cycloxydim |
| B.4 | cyhalofop-butyl |
| B.5 | fenoxaprop-ethyl |
| B.6 | fenoxaprop-P-ethyl |
| B.7 | metamifop |
| B.8 | pinoxaden |
| B.9 | profoxydim |
| B.10 | sethoxydim |
| B.11 | tepraloxydim |
| B.12 | tralkoxydim |
| B.13 | esprocarb |
| B.14 | ethofumesate |
| B.15 | molinate |
| B.16 | prosulfocarb |
| B.17 | thiobencarb |
| B.18 | triallate |
| B.19 | bensulfuron-methyl |
| B.20 | bispyribac-sodium |
| B.21 | cloransulam-methyl |
| B.22 | chlorsulfuron |
| B.23 | clorimuron |
| B.24 | cyclosulfamuron |
| B.25 | diclosulam |
| B.26 | florasulam |
| B.27 | flumetsulam |
| B.28 | flupyrsulfuron-methyl-sodium |
| B.29 | foramsulfuron |
| B.30 | imazamox |
| B.31 | imazamox-ammonium |
| B.32 | imazapic |
| B.33 | imazapic-ammonium |
| B.34 | imazapic-isopropylammonium |
| B.35 | imazapyr |
| B.36 | imazapyr-ammonium |
| B.37 | imazapyr-isopropylammonium |
| B.38 | imazaquin |
| B.39 | imazaquin-ammonium |
| B.40 | imazethapyr |
| B.41 | imazethapyr-ammonium |
| B.42 | imazethapyr-isopropylammonium |
| B.43 | imazosulfuron |
| B.44 | iodosulfuron-methyl-sodium |
| B.45 | iofensulfuron |
| B.46 | iofensulfuron-sodium |
| B.47 | mesosulfuron-methyl |
| B.48 | metazosulfuron |
| B.49 | metsulfuron-methyl |
| B.50 | metosulam |
| B.51 | nicosulfuron |
| B.52 | penoxsulam |
| B.53 | propoxycarbazon-sodium |
| B.54 | pyrazosulfuron-ethyl |
| B.55 | pyribenzoxim |
| B.56 | pyriftalid |
| B.57 | pyroxsulam |
| B.58 | propyrisulfuron |
| B.59 | rimsulfuron |
| B.60 | sulfosulfuron |
| B.61 | thiencarbazone-methyl |
| B.62 | thifensulfuron-methyl |
| B.63 | tribenuron-methyl |
| B.64 | tritosulfuron |
| B.65 | triafamone |
| B.66 | ametryne |
| B.67 | atrazine |
| B.68 | bentazon |
| B.69 | bromoxynil |
| B.70 | bromoxynil-octanoate |
| B.71 | bromoxynil-heptanoate |
| B.72 | bromoxynil-potassium |
| B.73 | diuron |
| B.74 | fluometuron |
| B.75 | hexazinone |
| B.76 | isoproturon |
| B.77 | linuron |
| B.78 | metamitron |
| B.79 | metribuzin |
| B.80 | propanil |
| B.81 | simazin |
| B.82 | terbuthylazine |
| B.83 | terbutryn |
| B.84 | paraquat-dichloride |
| B.85 | acifluorfen |
| B.86 | butafenacil |
| B.87 | carfentrazone-ethyl |
| B.88 | flumioxazin |
| B.89 | fomesafen |
| B.90 | oxadiargyl |
| B.91 | oxyfluorfen |
| B.92 | pyraflufen |
| B.93 | pyraflufen-ethyl |
| B.94 | saflufenacil |
| B.95 | sulfentrazone |
| B.96 | trifludimoxazin |
| B.97 | ethyl [3-[2-chloro-4-fluoro-5-(1-methyl-6-trifluoromethyl- |
| | 2,4-di-oxo-1,2,3,4-tetrahydropyrimidin-3-yl)phenoxy]-2- |
| | pyridyloxy]acetate (CAS 353292-31-6) |
| B.98 | benzobicyclon |
| B.99 | bicyclopyrone |
| B.100 | clomazone |
| B.101 | diflufenican |
| B.102 | flurochloridone |
| B.103 | isoxaflutole |
| B.104 | mesotrione |
| B.105 | norflurazone |
| B.106 | picolinafen |
| B.107 | sulcotrione |
| B.108 | tefuryltrione |
| B.109 | tembotrione |
| B.110 | tolpyralate |
| B.111 | topramezone |
| B.112 | topramezone-sodium |
| B.113 | amitrole |
| B.114 | fluometuron |
| B.115 | fenquinotrione |
| B.116 | glyphosate |
| B.117 | glyphosate-ammonium |
| B.118 | glyphosate-dimethylammonium |
| B.119 | glyphosate-isopropylammonium |
| B.120 | glyphosate-trimesium (sulfosate) |
| B.121 | glyphosate-potassium |
| B.122 | glufosinate |
| B.123 | glufosinate-ammonium |
| B.124 | glufosinate-P |
| B.125 | glufosinate-P-ammonium |
| B.126 | pendimethalin |
| B.127 | trifluralin |
| B.128 | acetochlor |
| B.129 | butachlor |
| B.130 | cafenstrole |
| B.131 | dimethenamid-P |
| B.132 | fentrazamide |
| B.133 | flufenacet |
| B.134 | mefenacet |
| B.135 | metazachlor |
| B.136 | metolachlor |
| B.137 | S-metolachlor |
| B.138 | pretilachlor |
| B.139 | fenoxasulfone |
| B.140 | indaziflam |
| B.141 | isoxaben |
| B.142 | triaziflam |
| B.143 | ipfencarbazone |
| B.144 | pyroxasulfone |
| B.145 | 2,4-D |
| B.146 | 2,4-D-isobutyl |
| B.147 | 2,4-D-dimethylammonium |
| B.148 | 2,4-D-N,N,N-trimethylethanolammonium |
| B.149 | aminopyralid |
| B.150 | aminopyralid-methyl |
| B.151 | aminopyralid-dimethylammonium |
| B.152 | aminopyralid-tris(2-hydroxypropyl)ammonium |
| B.153 | clopyralid |
| B.154 | clopyralid-methyl |
| B.155 | clopyralid-olamine |
| B.156 | dicamba |
| B.157 | dicamba-butotyl |
| B.158 | dicamba-diglycolamine |
| B.159 | dicamba-dimethylammonium |
| B.160 | dicamba-diolamine |
| B.161 | dicamba-isopropylammonium |
| B.162 | dicamba-potassium |
| B.163 | dicamba-sodium |
| B.164 | dicamba-trolamine |
| B.165 | dicamba-N,N-bis-(3-aminopropyl)methylamine |
| B.166 | dicamba-diethylenetriamine |
| B.167 | fluroxypyr |
| B.168 | fluroxypyr-meptyl |
| B.169 | halauxifen |
| B.170 | halauxifen-methyl |
| B.171 | MCPA |
| B.172 | MCPA-2-ethylhexyl |
| B.173 | MCPA-dimethylammonium |
| B.174 | quinclorac |
| B.175 | quinclorac-dimethylammonium |
| B.176 | quinmerac |
| B.177 | quinmerac-dimethylammonium |
| B.178 | florpyrauxifen |
| B.179 | florpyrauxifen-benzyl (CAS 1390661-72-9) |
| B.180 | aminocyclopyrachlor |
| B.181 | aminocyclopyrachlor-potassium |
| B.182 | aminocyclopyrachlor-methyl |
| B.183 | diflufenzopyr |
| B.184 | diflufenzopyr-sodium |
| B.185 | dymron |
| B.186 | indanofan |
| B.187 | oxaziclomefone |
| B.188 | II.1 |
| B.189 | II.2 |
| B.190 | II.3 |
| B.191 | II.4 |
| B.192 | II.5 |
| B.193 | II.6 |
| B.194 | II.7 |
| B.195 | II.8 |
| B.196 | II.9 |
| B.197 | 4-amino-3-chloro-5-fluoro-6-(7-fluoro-1H-indol-6- |
| | yl)picolinic acid (CAS 1629965-65-6) |
| B.198 | flopyrauxifen |
| B.199 | oxotrione (CAS 1486617-21-3) |
| B.200 | cinmethylin |
| B.201 | 2-chloro-3-methylsulfanyl-N-(1-methyltetrazol-5-yl)- |
| | 4-(trifluoro-methyl)benzamide (CAS 1361139-71-0) |
| B.202 | 2-(2,4-dichlorophenyl)methyl-4,4-dimethyl-3-isoxazolidone |
| | (CAS 81777-95-9) |

In another embodiment of the present invention the compositions according to the present invention comprise at least one pyrimidine compound of formula (I) and at least one safener C.

Safeners are chemical compounds which prevent or reduce damage on useful plants without having a major impact on the herbicidal action of the herbicidal active components of the present compositions towards unwanted plants. They can be applied either before sowings (e.g. on seed treatments, shoots or seedlings) or in the pre-emergence application or post-emergence application of the useful plant. The safeners and the pyrimidine compounds of formula (I) and/or the herbicides B can be applied simultaneously or in succession.

Suitable safeners are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenylcarbamates and their agriculturally acceptable salts and their agriculturally acceptable derivatives such amides, esters, and thioesters, provided they have an acid group.

Examples of preferred safeners C are benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4), metcamifen and BPCMS (CAS 54091-06-4). Especially preferred safeners C are benoxacor, cloquintocet, cyprosulfamide, dichlormid, fenchlorazole, fenclorim, flurazole, fluxofenim, furilazole, isoxadifen, mefenpyr, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and metcamifen.

Particularly preferred safeners C are benoxacor, cloquintocet, cyprosulfamide, dichlormid, fenchlorazole, fenclorim, furilazole, isoxadifen, mefenpyr, naphtalic anhydride, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and metcamifen.

Particularly preferred safeners C, which, as component C, are constituent of the composition according to the invention are the safeners C as defined above; in particular the safeners C.1-C.17 listed below in table C:

TABLE C

| C | Safener C |
|---|---|
| C.1 | benoxacor |
| C.2 | cloquintocet |
| C.3 | cloquintocet-mexyl |
| C.4 | cyprosulfamide |
| C.5 | dichlormid |
| C.6 | fenchlorazole |
| C.7 | fenchlorazole-ethyl |
| C.8 | fenclorim |
| C.9 | furilazole |
| C.10 | isoxadifen |
| C.11 | isoxadifen-ethyl |
| C.12 | mefenpyr |
| C.13 | mefenpyr-diethyl |
| C.14 | naphtalic acid anhydride |
| C.15 | 4-(dichloroacetyl)-1-oxa-4-azaspiro[4.5]decane |
| | (MON4660, CAS 71526-07-3) |
| C.16 | 2,2,5-trimethyl-3-(dichloroacetyl)-1,3-oxazolidine |
| | (R-29148, CAS 52836-31-4) |
| C.17 | metcamifen |

The active compounds B of groups b1) to b15) and the active compounds C are known herbicides and safeners, see, e.g., The Compendium of Pesticide Common Names (http://www.alanwood.net/pesticides/); Farm Chemicals Handbook 2000 volume 86, Meister Publishing Company, 2000; B. Hock, C. Fedtke, R. R. Schmidt, Herbizide [Herbicides], Georg Thieme Verlag, Stuttgart 1995; W. H. Ahrens, Herbicide Handbook, 7th edition, Weed Science Society of America, 1994; and K. K. Hatzios, Herbicide Handbook, Supplement for the 7th edition, Weed Science Society of America, 1998. 2,2,5-Trimethyl-3-(dichloroacetyl)-1,3-oxazolidine [CAS No. 52836-31-4] is also referred to as R-29148. 4-(Dichloroacetyl)-1-oxa-4-azaspiro[4.5]decane [CAS No. 71526-07-3] is also referred to as AD-67 and MON 4660.

The assignment of the active compounds to the respective mechanisms of action is based on current knowledge. If several mechanisms of action apply to one active compound, this substance was only assigned to one mechanism of action.

Active compounds B and C having a carboxyl group can be employed in the form of the acid, in the form of an agriculturally suitable salt as mentioned above or else in the form of an agriculturally acceptable derivative in the compositions according to the invention.

In the case of dicamba, suitable salts include those, where the counterion is an agriculturally acceptable cation. E.g., suitable salts of dicamba are dicamba-sodium, dicamba-potassium, dicamba-methylammonium, dicamba-dimethylammonium, dicamba-isopropylammonium, dicamba-diglycolamine, dicamba-olamine, dicamba-diolamine, dicamba-trolamine, dicamba-N,N-bis-(3-aminopropyl)methylamine and dicamba-diethylenetriamine. Examples of a suitable ester are dicamba-methyl and dicamba-butotyl.

Suitable salts of 2,4-D are 2,4-D-ammonium, 2,4-D-dimethylammonium, 2,4-D-diethylammonium, 2,4-D-diethanolammonium (2,4-D-diolamine), 2,4-D-triethanolammonium, 2,4-D-isopropylammonium, 2,4-D-triisopropanolammonium, 2,4-D-heptylammonium, 2,4-D-dodecylammonium, 2,4-D-tetradecylammonium, 2,4-D-triethylammonium, 2,4-D-tris(2-hydroxypropyl)ammonium, 2,4-D-tris(isopropyl)ammonium, 2,4-D-trolamine, 2,4-D-lithium, 2,4-D-sodium. Examples of suitable esters of 2,4-D are 2,4-D-butotyl, 2,4-D-2-butoxypropyl, 2,4-D-3-butoxypropyl, 2,4-D-butyl, 2,4-D-ethyl, 2,4-D-ethylhexyl, 2,4-D-isobutyl, 2,4-D-isooctyl, 2,4-D-isopropyl, 2,4-D-meptyl, 2,4-D-methyl, 2,4-D-octyl, 2,4-D-pentyl, 2,4-D-propyl, 2,4-D-tefuryl and clacyfos.

Suitable salts of 2,4-DB are e.g. 2,4-DB-sodium, 2,4-DB-potassium and 2,4-DB-dimethylammonium. Suitable esters of 2,4-DB are e.g. 2,4-DB-butyl and 2,4-DB-isoctyl.

Suitable salts of dichlorprop are e.g. dichlorprop-sodium, dichlorprop-potassium and dichlorprop-dimethylammonium. Examples of suitable esters of dichlorprop are dichlorprop-butotyl and dichlorprop-isoctyl.

Suitable salts and esters of MCPA include MCPA-butotyl, MCPA-butyl, MCPA-dimethylammonium, MCPA-diolamine, MCPA-ethyl, MCPA-thioethyl, MCPA-2-ethylhexyl, MCPA-isobutyl, MCPA-isoctyl, MCPA-isopropyl, MCPA-isopropylammonium, MCPA-methyl, MCPA-olamine, MCPA-potassium, MCPA-sodium and MCPA-trolamine.

A suitable salt of MCPB is MCPB sodium. A suitable ester of MCPB is MCPB-ethyl.

Suitable salts of clopyralid are clopyralid-potassium, clopyralid-olamine and clopyralid-tris-(2-hydroxypropyl)ammonium. Example of suitable esters of clopyralid is clopyralid-methyl.

Examples of a suitable ester of fluroxypyr are fluroxypyr-meptyl and fluroxypyr-2-butoxy-1-methylethyl, wherein fluroxypyr-meptyl is preferred.

Suitable salts of picloram are picloram-dimethylammonium, picloram-potassium, picloram-triisopropanolammonium, picloram-triisopropylammonium and picloram-trolamine. A suitable ester of picloram is picloram-isoctyl.

A suitable salt of triclopyr is triclopyr-triethylammonium. Suitable esters of triclopyr are e.g. triclopyr-ethyl and triclopyr-butotyl.

Suitable salts and esters of chloramben include chloramben-ammonium, chloramben-diolamine, chloramben-methyl, chloramben-methylammonium and chloramben-sodium. Suitable salts and esters of 2,3,6-TBA include 2,3,6-TBA-dimethylammonium, 2,3,6-TBA-lithium, 2,3,6-TBA-potassium and 2,3,6-TBA-sodium.

Suitable salts and esters of aminopyralid include aminopyralid-potassium, aminopyralid-dimethylammonium, and aminopyralid-tris(2-hydroxypropyl)ammonium.

Suitable salts of glyphosate are e.g. glyphosate-ammonium, glyphosate-diammonium, glyphoste-dimethylammonium, glyphosate-isopropylammonium, glyphosate-potassium, glyphosate-sodium, glyphosate-trimesium as well as the ethanolamine and diethanolamine salts, preferably glyphosate-diammonium, glyphosate-isopropylammonium and glyphosate-trimesium (sulfosate).

A suitable salt of glufosinate is e.g. glufosinate-ammonium.

A suitable salt of glufosinate-P is e.g. glufosinate-P-ammonium.

Suitable salts and esters of bromoxynil are e.g. bromoxynil-butyrate, bromoxynil-heptanoate, bromoxynil-octanoate, bromoxynil-potassium and bromoxynil-sodium.

Suitable salts and esters of ioxonil are e.g. ioxonil-octanoate, ioxonil-potassium and ioxonil-sodium.

Suitable salts and esters of mecoprop include mecoprop-butotyl, mecoprop-dimethylammonium, mecoprop-diolamine, mecoprop-ethadyl, mecoprop-2-ethylhexyl, mecoprop-isoctyl, mecoprop-methyl, mecoprop-potassium, mecoprop-sodium and mecoprop-trolamine.

Suitable salts of mecoprop-P are e.g. mecoprop-P-butotyl, mecoprop-P-dimethylammonium, mecoprop-P-2-ethylhexyl, mecoprop-P-isobutyl, mecoprop-P-potassium and mecoprop-P-sodium.

A suitable salt of diflufenzopyr is e.g. diflufenzopyr-sodium.

A suitable salt of naptalam is e.g. naptalam-sodium.

Suitable salts and esters of aminocyclopyrachlor are e.g. aminocyclopyrachlor-dimethylammonium, aminocyclopyrachlor-methyl, aminocyclopyrachlor-triisopropanolammonium, aminocyclopyrachlor-sodium and aminocyclopyrachlor-potassium.

A suitable salt of quinclorac is e.g. quinclorac-dimethylammonium.

A suitable salt of quinmerac is e.g. quinmerac-dimethylammonium.

A suitable salt of imazamox is e.g. imazamox-ammonium.

Suitable salts of imazapic are e.g. imazapic-ammonium and imazapic-isopropylammonium.

Suitable salts of imazapyr are e.g. imazapyr-ammonium and imazapyr-isopropylammonium.

A suitable salt of imazaquin is e.g. imazaquin-ammonium.

Suitable salts of imazethapyr are e.g. imazethapyr-ammonium and imazethapyr-isopropylammonium.

A suitable salt of topramezone is e.g. topramezone-sodium.

According to a preferred embodiment of the invention, the composition comprises as herbicidal active compound B or component B at least one, preferably exactly one herbicide B.

According to another preferred embodiment of the invention, the composition comprises as herbicidal active compounds B or component B at least two, preferably exactly two herbicides B different from each other.

According to another preferred embodiment of the invention, the composition comprises as herbicidal active compounds B or component B at least three, preferably exactly three herbicides B different from each other.

According to another preferred embodiment of the invention, the composition comprises as safening component C or component C at least one, preferably exactly one safener C.

According to another preferred embodiment of the invention, the composition comprises as component B at least one, preferably exactly one herbicide B, and as component C at least one, preferably exactly one, safener C.

According to another preferred embodiment of the invention, the composition comprises at least two, preferably exactly two, herbicides B different from each other, and as component C at least one, preferably exactly one, safener C.

According to another preferred embodiment of the invention, the composition comprises at least three, preferably exactly three, herbicides B different from each other, and as component C at least one, preferably exactly one, safener C.

According to another preferred embodiment of the invention, the composition comprises as component A at least one, preferably exactly one pyrimidine compound of formula (I), preferably of formula (I.1), especially preferred the compound (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), or (1.1.I-67), and as component B at least one, preferably exactly one, herbicide B.

According to another preferred embodiment of the invention, the composition comprises as component A at least one, preferably exactly one pyrimidine compound of formula (I), preferably of formula (I.1), especially preferred the compound (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), or (1.1.I-67), and at least two, preferably exactly two, herbicides B different from each other.

According to another preferred embodiment of the invention, the composition comprises as component A at least one, preferably exactly one pyrimidine compound of formula (I), preferably of formula (I.1), especially preferred the compound (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), or (1.1.I-67), and at least three, preferably exactly three, herbicides B different from each other.

According to another preferred embodiment of the invention, the composition comprises as component A at least one, preferably exactly one pyrimidine compound of formula (I), preferably of formula (I.1), especially preferred the compound (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), or (1.1.I-67), and as component C at least one, preferably exactly one, safener C.

According to another preferred embodiment of the invention, the composition comprises as component A at least one, preferably exactly one pyrimidine compound of formula (I), preferably of formula (I.1), especially preferred the compound (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), or (1.1.I-67), as component B at least one, preferably exactly one, herbicide B, and as component C at least one, preferably exactly one safener C.

According to another preferred embodiment of the invention, the composition comprises as component A at least one, preferably exactly pyrimidine compound of formula (I), preferably of formula (I.1), especially preferred the compound (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), or (1.1.I-67), at least two, preferably exactly two herbicides B different from each other, and as component C at least one, preferably exactly one, safener C.

According to another preferred embodiment of the invention, the composition comprises as component A at least one, preferably exactly one pyrimidine compound of formula (I), preferably of formula (I.1), especially preferred the compound (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), or (1.1.I-67), at least three, preferably exactly three herbicides B different from each other, and as component C at least one, preferably exactly one, safener C.

According to another preferred embodiment of the invention, the composition comprises as component A at least one, preferably exactly one pyrimidine compound of formula (I), preferably of formula (I.2), especially preferred the compound (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), or (1.2.I-67), and as component B at least one, preferably exactly one, herbicide B.

According to another preferred embodiment of the invention, the composition comprises as component A at least one, preferably exactly one pyrimidine compound of formula (I), preferably of formula (I.2), especially preferred the compound (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), or (1.2.I-67), and at least two, preferably exactly two, herbicides B different from each other.

According to another preferred embodiment of the invention, the composition comprises as component A at least one, preferably exactly one pyrimidine compound of formula (I), preferably of formula (I.2), especially preferred the compound (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), or (1.2.I-67), and at least three, preferably exactly three, herbicides B different from each other.

According to another preferred embodiment of the invention, the composition comprises as component A at least one, preferably exactly one pyrimidine compound of formula (I), preferably of formula (I.2), especially preferred the compound (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), or (1.2.I-67), and as component C at least one, preferably exactly one, safener C.

According to another preferred embodiment of the invention, the composition comprises as component A at least one, preferably exactly one pyrimidine compound of formula (I), preferably of formula (I.2), especially preferred the compound (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), or (1.2.I-67), as component B at least one, preferably exactly one, herbicide B, and as component C at least one, preferably exactly one safener C.

According to another preferred embodiment of the invention, the composition comprises as component A at least one, preferably exactly pyrimidine compound of formula (I), preferably of formula (I.2), especially preferred the compound (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), or (1.2.I-67), at least two, preferably exactly two herbicides B different from each other, and as component C at least one, preferably exactly one, safener C.

According to another preferred embodiment of the invention, the composition comprises as component A at least one, preferably exactly one pyrimidine compound of formula (I), preferably of formula (I.2), especially preferred the compound (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), or (1.2.I-67), at least three, preferably exactly three herbicides B different from each other, and as component C at least one, preferably exactly one, safener C.

According to another preferred embodiment of the invention, the composition comprises as component A at least one, preferably exactly one pyrimidine compound of formula (I), preferably of formula (I.5), especially preferred the compound (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), or (1.5.I-67), and as component B at least one, preferably exactly one, herbicide B.

According to another preferred embodiment of the invention, the composition comprises as component A at least one, preferably exactly one pyrimidine compound of formula (I), preferably of formula (I.5), especially preferred the compound (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), or (1.5.I-67), and at least two, preferably exactly two, herbicides B different from each other.

According to another preferred embodiment of the invention, the composition comprises as component A at least one, preferably exactly one pyrimidine compound of formula (I), preferably of formula (I.5), especially preferred the compound (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), or (1.5.I-67), and at least three, preferably exactly three, herbicides B different from each other.

According to another preferred embodiment of the invention, the composition comprises as component A at least one, preferably exactly one pyrimidine compound of formula (I), preferably of formula (I.5), especially preferred the compound (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), or (1.5.I-67), and as component C at least one, preferably exactly one, safener C.

According to another preferred embodiment of the invention, the composition comprises as component A at least one, preferably exactly one pyrimidine compound of formula (I), preferably of formula (I.5), especially preferred the compound (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), or (1.5.I-67), as component B at least one, preferably exactly one, herbicide B, and as component C at least one, preferably exactly one safener C.

According to another preferred embodiment of the invention, the composition comprises as component A at least one, preferably exactly pyrimidine compound of formula (I), preferably of formula (I.5), especially preferred the compound (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), or (1.5.I-67), at least two, preferably exactly two herbicides B different from each other, and as component C at least one, preferably exactly one, safener C.

According to another preferred embodiment of the invention, the composition comprises as component A at least one, preferably exactly one pyrimidine compound of formula (I), preferably of formula (I.5), especially preferred the compound (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), or (1.5.I-67), at least three, preferably exactly three herbicides B different from each other, and as component C at least one, preferably exactly one, safener C.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b1), in particular selected from the group consisting of clethodim, clodinafop-propargyl, cycloxydim, cyhalofop-butyl, fenoxaprop-ethyl, fenoxaprop-P-ethyl, metamifop, pinoxaden, profoxydim, sethoxydim, tepraloxydim, tralkoxydim, esprocarb, ethofumesate, molinate, prosulfocarb, thiobencarb and triallate.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b2), in particular selected from the group consisting of bensulfuron-methyl, bispyribac-sodium, cloransulam-methyl, chlorsulfuron, clorimuron, cyclosulfamuron, diclosulam, florasulam, flumetsulam, flupyrsulfuron-methyl-sodium, foramsulfuron, imazamox, imazamox-ammonium, imazapic, imazapic-ammonium, imazapic-isopropylammonium, imazapyr, imazapyr-ammonium, imazethapyr-isopropylammonium, imazaquin, imazaquin-ammonium, imazethapyr, imazethapyr-ammonium, imazethapyr-isopropylammonium, imazosulfuron, iodosulfuron-methyl-sodium, iofensulfuron, iofensulfuron-sodium, mesosulfuron-methyl, metazosulfuron, metsulfuron-methyl, metosulam, nicosulfuron, penoxsulam, propoxycarbazon-sodium, pyrazosulfuron-ethyl, pyribenzoxim, pyriftalid, pyroxsulam, propyrisulfuron, rimsulfuron, sulfosulfuron, thiencarbazon-methyl, thifensulfuron-methyl, tribenuron-methyl, tritosulfuron and triafamone.

According to another preferred embodiment of the invention, the composition comprises, in addition to a pyrimidine compounds of formula (I), especially an active compound from the group consisting (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b3), in particular selected from the group consisting of ametryn, atrazine, bentazon, bromoxynil, bromoxynil-octanoate, bromoxynil-heptanoate, bromoxynil-potassium, diuron, fluometuron, hexazinone, isoproturon, linuron, metamitron, metribuzin, paraquat-dichloride, propanil, simazin, terbutryn and terbuthylazine.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b4), in particular selected from the group consisting of acifluorfen, butafencil, carfenetrazone-ethyl, flumioxazin, fomesafen, oxadiargyl, oxyfluorfen, pyraflufen, pyraflufen-ethyl, saflufenacil, sulfentrazone, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100).

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b5), in particular selected from the group consisting of amitrole, benzobicyclon, bicyclopyrone, clomazone, diflufenican, fenquintrone, fluometuron, flurochloridone, isoxaflutole, mesotrione, norflurazone, oxotrione (CAS 1486617-21-3), picolinafen, sulcotrione, tefuryltrione, tembotrione, tolpyralate, topramezone, topramezone-sodium and 2-chloro-3-methylsulfanyl-N-(1-methyltetrazol-5-yl)-4-(trifluoro-methyl)benzamide (CAS 1361139-71-0).

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b6), in particular selected from the group consisting of glyphosate, glyphosate-ammonium, glyphosate-dimethylammonium, glyphosate-isopropylammonium and glyphosate-trimesium (sulfosate) and glyphosate-potassium.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b7), in particular selected from the group consisting of glufosinate, glufosinate-ammonium, glufosinate-P and glufosinate-P-ammonium.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b9), in particular selected from the group consisting of pendimethalin and trifluralin.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b10), in particular selected from the group consisting of acetochlor, butachlor, cafenstrole, dimethenamid-P, fentrazamide, flufenacet, mefenacet, metazachlor, metolachlor, S-metolachlor, fenoxasulfone, ipfencarbazone and pyroxasulfone. Likewise, preference is given to compositions comprising in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b10), in particular selected from the group consisting of isoxazoline compounds of the formulae II.1, II.2, II.3, II.4, II.5, II.6, II.7, II.8 and II.9, as defined above.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b11), in particular indaziflam, isoxaben and triaziflam.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b13), in particular selected from the group consisting of 2,4-D, 2,4-D-isobutyl, 2,4-D-dimethylammonium, 2,4-D-N,N,N-trimethylethanolammonium, aminocyclopyrachlor, aminocyclopyrachlor-potassium, aminocyclopyrachlor-methyl, aminopyralid, aminopyralid-methyl, aminopyralid-dimethylammonium, aminopyralid-tris(2-hydroxypropyl)ammonium, clopyralid, clopyralid-methyl, clopyralid-olamine, dicamba, dicamba-butotyl, dicamba-diglycolamine, dicamba-dimethylammonium, dicamba-diolamine, dicamba-isopropylammonium, dicamba-potassium, dicamba-sodium, dicamba-trolamine, dicamba-N,N-bis-(3-aminopropyl)methylamine, dicamba-diethylenetriamine, flopyrauxifen, fluroxypyr, fluroxypyr-meptyl, halauxifen, halauxifen-methyl, MCPA, MCPA-2-ethylhexyl, MCPA-dimethylammonium, quinclorac, quinclorac-dimethylammonium, quinmerac, quinmerac-dimethylammonium, florpyrauxifen, florpyrauxifen-benzyl (CAS 1390661-72-9), and 4-amino-3-chloro-5-fluoro-6-(7-fluoro-1H-indol-6-yl)picolinic acid (CAS 1629965-65-6).

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b14), in particular selected from the group consisting of diflufenzopyr, diflufenzopyr-sodium, dymron, indanofan and diflufenzopyr-sodium.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one herbicidally active compound from group b15), in particular selected from the group consisting of cinmethylin, dymron (=daimuron), indanofan and oxaziclomefone.

According to another preferred embodiment of the invention, the composition comprises, in addition to a pyrimidine compounds of formula (I), especially an active compound from the group consisting of (1.1.I-3), (1.1.I-4), (1.1.I-10), (1.1.I-11), (1.1.I-17), (1.1.I-18), (1.1.I-24), (1.1.I-25), (1.1.I-31), (1.1.I-32), (1.1.I-38), (1.1.I-39), (1.1.I-45), (1.1.I-46), (1.1.I-52), (1.1.I-53), (1.1.I-59), (1.1.I-60), (1.1.I-66), (1.1.I-67), (1.2.I-3), (1.2.I-4), (1.2.I-10), (1.2.I-11), (1.2.I-17), (1.2.I-18), (1.2.I-24), (1.2.I-25), (1.2.I-31), (1.2.I-32), (1.2.I-38), (1.2.I-39), (1.2.I-45), (1.2.I-46), (1.2.I-52), (1.2.I-53), (1.2.I-59), (1.2.I-60), (1.2.I-66), (1.2.I-67), (1.5.I-3), (1.5.I-4), (1.5.I-10), (1.5.I-11), (1.5.I-17), (1.5.I-18), (1.5.I-24), (1.5.I-25), (1.5.I-31), (1.5.I-32), (1.5.I-38), (1.5.I-39), (1.5.I-45), (1.5.I-46), (1.5.I-52), (1.5.I-53), (1.5.I-59), (1.5.I-60), (1.5.I-66), and (1.5.I-67), at least one and especially exactly one safener C, in particular selected from the group consisting of benoxacor, cloquintocet, cyprosulfamide, dichlormid, fenchlorazole, fenclorim, furilazole, isoxadifen, mefenpyr, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Here and below, the term “binary compositions” includes compositions comprising one or more, e.g. 1, 2 or 3, active compounds of the formula (I) and either one or more, e.g. 1, 2 or 3, herbicides B or one or more safeners C.

Correspondingly, the term “ternary compositions” includes compositions comprising one or more, e.g. 1, 2 or 3, active compounds of the formula (I), one or more, e.g. 1, 2 or 3, herbicides B and one or more, e.g. 1, 2 or 3, safeners C.

In binary compositions comprising at least one pyrimidine of formula (I) as component A and at least one herbicide B, the weight ratio of the active compounds A:B is generally in the range of from 1:1000 to 1000:1, preferably in the range of from 1:500 to 500:1, in particular in the range of from 1:250 to 250:1 and particularly preferably in the range of from 1:75 to 75:1.

In binary compositions comprising at least one pyrimidine of formula (I) as component A and at least one safener C, the weight ratio of the active compounds A:C is generally in the range of from 1:1000 to 1000:1, preferably in the range of from 1:500 to 500:1, in particular in the range of from 1:250 to 250:1 and particularly preferably in the range of from 1:75 to 75:1.

In ternary compositions comprising at least one pyrimidine of formula (I) as component A, at least one herbicide B and at least one safener C, the relative proportions by weight of the components A:B are generally in the range of from 1:1000 to 1000:1, preferably in the range of from 1:500 to 500:1, in particular in the range of from 1:250 to 250:1 and particularly preferably in the range of from 1:75 to 75:1, the weight ratio of the components A:C is generally in the range of from 1:1000 to 1000:1, preferably in the range of from 1:500 to 500:1, in particular in the range of from 1:250 to 250:1 and particularly preferably in the range of from 1:75 to 75:1, and the weight ratio of the components B:C is generally in the range of from 1:1000 to 1000:1, preferably in the range of from 1:500 to 500:1, in particular in the range of from 1:250 to 250:1 and particularly preferably in the range of from 1:75 to 75:1. The weight ratio of components A+B to component C is preferably in the range of from 1:500 to 500:1, in particular in the range of from 1:250 to 250:1 and particularly preferably in the range of from 1:75 to 75:1.

The weight ratios of the individual components in the preferred mixtures mentioned below are within the limits given above, in particular within the preferred limits.

Particularly preferred are the compositions mentioned below comprising the pyrimidine compounds of formula I as defined and the substance(s) as defined in the respective row of table T;

especially preferred comprising as only herbicidal active compounds the pyrimidine compounds of formula I as defined and the substance(s) as defined in the respective row of table T;

most preferably comprising as only active compounds the pyrimidine compounds of formula 1 as defined and the substance(s) as defined in the respective row of table T.

Particularly preferred are compositions 1.1 to 1.3653, comprising the compound 1.3.I-3 and the substance(s) as defined in the respective row of table T:

TABLE T
(compositions 1.1 to 1.3653):

| comp. no. | herbicide B | safener C |
|---|---|---|
| 1.1 | B.1 | — |
| 1.2 | B.2 | — |
| 1.3 | B.3 | — |
| 1.4 | B.4 | — |
| 1.5 | B.5 | — |
| 1.6 | B.6 | — |
| 1.7 | B.7 | — |
| 1.8 | B.8 | — |
| 1.9 | B.9 | — |
| 1.10 | B.10 | — |
| 1.11 | B.11 | — |
| 1.12 | B.12 | — |
| 1.13 | B.13 | — |
| 1.14 | B.14 | — |
| 1.15 | B.15 | — |
| 1.16 | B.16 | — |
| 1.17 | B.17 | — |
| 1.18 | B.18 | — |
| 1.19 | B.19 | — |
| 1.20 | B.20 | — |
| 1.21 | B.21 | — |
| 1.22 | B.22 | — |
| 1.23 | B.23 | — |
| 1.24 | B.24 | — |
| 1.25 | B.25 | — |
| 1.26 | B.26 | — |
| 1.27 | B.27 | — |
| 1.28 | B.28 | — |
| 1.29 | B.29 | — |
| 1.30 | B.30 | — |
| 1.31 | B.31 | — |
| 1.32 | B.32 | — |
| 1.33 | B.33 | — |
| 1.34 | B.34 | — |
| 1.35 | B.35 | — |
| 1.36 | B.36 | — |
| 1.37 | B.37 | — |
| 1.38 | B.38 | — |
| 1.39 | B.39 | — |
| 1.40 | B.40 | — |
| 1.41 | B.41 | — |
| 1.42 | B.42 | — |
| 1.43 | B.43 | — |
| 1.44 | B.44 | — |
| 1.45 | B.45 | — |
| 1.46 | B.46 | — |
| 1.47 | B.47 | — |
| 1.48 | B.48 | — |
| 1.49 | B.49 | — |
| 1.50 | B.50 | — |
| 1.51 | B.51 | — |
| 1.52 | B.52 | — |
| 1.53 | B.53 | — |
| 1.54 | B.54 | — |
| 1.55 | B.55 | — |
| 1.56 | B.56 | — |
| 1.57 | B.57 | — |
| 1.58 | B.58 | — |
| 1.59 | B.59 | — |
| 1.60 | B.60 | — |
| 1.61 | B.61 | — |
| 1.62 | B.62 | — |
| 1.63 | B.63 | — |
| 1.64 | B.64 | — |
| 1.65 | B.65 | — |
| 1.66 | B.66 | — |
| 1.67 | B.67 | — |
| 1.68 | B.68 | — |
| 1.69 | B.69 | — |
| 1.70 | B.70 | — |
| 1.71 | B.71 | — |
| 1.72 | B.72 | — |
| 1.73 | B.73 | — |
| 1.74 | B.74 | — |
| 1.75 | B.75 | — |
| 1.76 | B.76 | — |
| 1.77 | B.77 | — |
| 1.78 | B.78 | — |
| 1.79 | B.79 | — |
| 1.80 | B.80 | — |
| 1.81 | B.81 | — |
| 1.82 | B.82 | — |
| 1.83 | B.83 | — |
| 1.84 | B.84 | — |
| 1.85 | B.85 | — |
| 1.86 | B.86 | — |
| 1.87 | B.87 | — |
| 1.88 | B.88 | — |
| 1.89 | B.89 | — |
| 1.90 | B.90 | — |
| 1.91 | B.91 | — |
| 1.92 | B.92 | — |
| 1.93 | B.93 | — |
| 1.94 | B.94 | — |
| 1.95 | B.95 | — |
| 1.96 | B.96 | — |
| 1.97 | B.97 | — |
| 1.98 | B.98 | — |
| 1.99 | B.99 | — |
| 1.100 | B.100 | — |
| 1.101 | B.101 | — |
| 1.102 | B.102 | — |
| 1.103 | B.103 | — |
| 1.104 | B.104 | — |
| 1.105 | B.105 | — |
| 1.106 | B.106 | — |
| 1.107 | B.107 | — |
| 1.108 | B.108 | — |
| 1.109 | B.109 | — |
| 1.110 | B.110 | — |
| 1.111 | B.111 | — |
| 1.112 | B.112 | — |
| 1.113 | B.113 | — |
| 1.114 | B.114 | — |
| 1.115 | B.115 | — |
| 1.116 | B.116 | — |
| 1.117 | B.117 | — |
| 1.118 | B.118 | — |
| 1.119 | B.119 | — |
| 1.120 | B.120 | — |
| 1.121 | B.121 | — |
| 1.122 | B.122 | — |
| 1.123 | B.123 | — |
| 1.124 | B.124 | — |
| 1.125 | B.125 | — |
| 1.126 | B.126 | — |
| 1.127 | B.127 | — |
| 1.128 | B.128 | — |
| 1.129 | B.129 | — |
| 1.130 | B.130 | — |
| 1.131 | B.131 | — |
| 1.132 | B.132 | — |
| 1.133 | B.133 | — |
| 1.134 | B.134 | — |
| 1.135 | B.135 | — |
| 1.136 | B.136 | — |
| 1.137 | B.137 | — |
| 1.138 | B.138 | — |
| 1.139 | B.139 | — |
| 1.140 | B.140 | — |
| 1.141 | B.141 | — |
| 1.142 | B.142 | — |
| 1.143 | B.143 | — |
| 1.144 | B.144 | — |
| 1.145 | B.145 | — |
| 1.146 | B.146 | — |
| 1.147 | B.147 | — |
| 1.148 | B.148 | — |
| 1.149 | B.149 | — |
| 1.150 | B.150 | — |
| 1.151 | B.151 | — |
| 1.152 | B.152 | — |
| 1.153 | B.153 | — |
| 1.154 | B.154 | — |
| 1.155 | B.155 | — |
| 1.156 | B.156 | — |
| 1.157 | B.157 | — |
| 1.158 | B.158 | — |
| 1.159 | B.159 | — |
| 1.160 | B.160 | — |
| 1.161 | B.161 | — |
| 1.162 | B.162 | — |
| 1.163 | B.163 | — |
| 1.164 | B.164 | — |
| 1.165 | B.165 | — |
| 1.166 | B.166 | — |
| 1.167 | B.167 | — |
| 1.168 | B.168 | — |
| 1.169 | B.169 | — |
| 1.170 | B.170 | — |
| 1.171 | B.171 | — |
| 1.172 | B.172 | — |
| 1.173 | B.173 | — |
| 1.174 | B.174 | — |
| 1.175 | B.175 | — |
| 1.176 | B.176 | — |
| 1.177 | B.177 | — |
| 1.178 | B.178 | — |
| 1.179 | B.179 | — |
| 1.180 | B.180 | — |
| 1.181 | B.181 | — |
| 1.182 | B.182 | — |
| 1.183 | B.183 | — |
| 1.184 | B.184 | — |
| 1.185 | B.185 | — |
| 1.186 | B.186 | — |
| 1.187 | B.187 | — |
| 1.188 | B.188 | — |
| 1.189 | B.189 | — |
| 1.190 | B.190 | — |
| 1.191 | B.191 | — |
| 1.192 | B.192 | — |
| 1.193 | B.193 | — |
| 1.194 | B.194 | — |
| 1.195 | B.195 | — |
| 1.196 | B.196 | — |
| 1.197 | B.197 | — |
| 1.198 | B.198 | — |
| 1.199 | B.199 | — |
| 1.200 | B.200 | — |
| 1.201 | B.201 | — |
| 1.202 | B.202 | — |
| 1.203 | B.1 | C.1 |
| 1.204 | B.2 | C.1 |
| 1.205 | B.3 | C.1 |
| 1.206 | B.4 | C.1 |
| 1.207 | B.5 | C.1 |
| 1.208 | B.6 | C.1 |
| 1.209 | B.7 | C.1 |
| 1.210 | B.8 | C.1 |
| 1.211 | B.9 | C.1 |
| 1.212 | B.10 | C.1 |
| 1.213 | B.11 | C.1 |
| 1.214 | B.12 | C.1 |
| 1.215 | B.13 | C.1 |
| 1.216 | B.14 | C.1 |
| 1.217 | B.15 | C.1 |
| 1.218 | B.16 | C.1 |
| 1.219 | B.17 | C.1 |
| 1.220 | B.18 | C.1 |
| 1.221 | B.19 | C.1 |
| 1.222 | B.20 | C.1 |
| 1.223 | B.21 | C.1 |
| 1.224 | B.22 | C.1 |
| 1.225 | B.23 | C.1 |
| 1.226 | B.24 | C.1 |
| 1.227 | B.25 | C.1 |
| 1.228 | B.26 | C.1 |
| 1.229 | B.27 | C.1 |
| 1.230 | B.28 | C.1 |
| 1.231 | B.29 | C.1 |
| 1.232 | B.30 | C.1 |
| 1.233 | B.31 | C.1 |
| 1.234 | B.32 | C.1 |
| 1.235 | B.33 | C.1 |
| 1.236 | B.34 | C.1 |
| 1.237 | B.35 | C.1 |
| 1.238 | B.36 | C.1 |
| 1.239 | B.37 | C.1 |
| 1.240 | B.38 | C.1 |
| 1.241 | B.39 | C.1 |
| 1.242 | B.40 | C.1 |
| 1.243 | B.41 | C.1 |
| 1.244 | B.42 | C.1 |
| 1.245 | B.43 | C.1 |
| 1.246 | B.44 | C.1 |
| 1.247 | B.45 | C.1 |
| 1.248 | B.46 | C.1 |
| 1.249 | B.47 | C.1 |
| 1.250 | B.48 | C.1 |
| 1.251 | B.49 | C.1 |
| 1.252 | B.50 | C.1 |
| 1.253 | B.51 | C.1 |
| 1.254 | B.52 | C.1 |
| 1.255 | B.53 | C.1 |
| 1.256 | B.54 | C.1 |
| 1.257 | B.55 | C.1 |
| 1.258 | B.56 | C.1 |
| 1.259 | B.57 | C.1 |
| 1.260 | B.58 | C.1 |
| 1.261 | B.59 | C.1 |
| 1.262 | B.60 | C.1 |
| 1.263 | B.61 | C.1 |
| 1.264 | B.62 | C.1 |
| 1.265 | B.63 | C.1 |
| 1.266 | B.64 | C.1 |
| 1.267 | B.65 | C.1 |
| 1.268 | B.66 | C.1 |
| 1.269 | B.67 | C.1 |
| 1.270 | B.68 | C.1 |
| 1.271 | B.69 | C.1 |
| 1.272 | B.70 | C.1 |
| 1.273 | B.71 | C.1 |
| 1.274 | B.72 | C.1 |
| 1.275 | B.73 | C.1 |
| 1.276 | B.74 | C.1 |
| 1.277 | B.75 | C.1 |
| 1.278 | B.76 | C.1 |
| 1.279 | B.77 | C.1 |
| 1.280 | B.78 | C.1 |
| 1.281 | B.79 | C.1 |
| 1.282 | B.80 | C.1 |
| 1.283 | B.81 | C.1 |
| 1.284 | B.82 | C.1 |
| 1.285 | B.83 | C.1 |
| 1.286 | B.84 | C.1 |
| 1.287 | B.85 | C.1 |
| 1.288 | B.86 | C.1 |
| 1.289 | B.87 | C.1 |
| 1.290 | B.88 | C.1 |
| 1.291 | B.89 | C.1 |
| 1.292 | B.90 | C.1 |
| 1.293 | B.91 | C.1 |
| 1.294 | B.92 | C.1 |
| 1.295 | B.93 | C.1 |
| 1.296 | B.94 | C.1 |
| 1.297 | B.95 | C.1 |
| 1.298 | B.96 | C.1 |
| 1.299 | B.97 | C.1 |
| 1.300 | B.98 | C.1 |
| 1.301 | B.99 | C.1 |
| 1.302 | B.100 | C.1 |
| 1.303 | B.101 | C.1 |
| 1.304 | B.102 | C.1 |
| 1.305 | B.103 | C.1 |
| 1.306 | B.104 | C.1 |
| 1.307 | B.105 | C.1 |
| 1.308 | B.106 | C.1 |
| 1.309 | B.107 | C.1 |
| 1.310 | B.108 | C.1 |
| 1.311 | B.109 | C.1 |
| 1.312 | B.110 | C.1 |
| 1.313 | B.111 | C.1 |
| 1.314 | B.112 | C.1 |
| 1.315 | B.113 | C.1 |
| 1.316 | B.114 | C.1 |
| 1.317 | B.115 | C.1 |
| 1.318 | B.116 | C.1 |
| 1.319 | B.117 | C.1 |
| 1.320 | B.118 | C.1 |
| 1.321 | B.119 | C.1 |
| 1.322 | B.120 | C.1 |
| 1.323 | B.121 | C.1 |
| 1.324 | B.122 | C.1 |
| 1.325 | B.123 | C.1 |
| 1.326 | B.124 | C.1 |
| 1.327 | B.125 | C.1 |
| 1.328 | B.126 | C.1 |
| 1.329 | B.127 | C.1 |
| 1.330 | B.128 | C.1 |
| 1.331 | B.129 | C.1 |
| 1.332 | B.130 | C.1 |
| 1.333 | B.131 | C.1 |
| 1.334 | B.132 | C.1 |
| 1.335 | B.133 | C.1 |
| 1.336 | B.134 | C.1 |
| 1.337 | B.135 | C.1 |
| 1.338 | B.136 | C.1 |
| 1.339 | B.137 | C.1 |
| 1.340 | B.138 | C.1 |
| 1.341 | B.139 | C.1 |
| 1.342 | B.140 | C.1 |
| 1.343 | B.141 | C.1 |
| 1.344 | B.142 | C.1 |
| 1.345 | B.143 | C.1 |
| 1.346 | B.144 | C.1 |
| 1.347 | B.145 | C.1 |
| 1.348 | B.146 | C.1 |
| 1.349 | B.147 | C.1 |
| 1.350 | B.148 | C.1 |
| 1.351 | B.149 | C.1 |
| 1.352 | B.150 | C.1 |
| 1.353 | B.151 | C.1 |
| 1.354 | B.152 | C.1 |
| 1.355 | B.153 | C.1 |
| 1.356 | B.154 | C.1 |
| 1.357 | B.155 | C.1 |
| 1.358 | B.156 | C.1 |
| 1.359 | B.157 | C.1 |
| 1.360 | B.158 | C.1 |
| 1.361 | B.159 | C.1 |
| 1.362 | B.160 | C.1 |
| 1.363 | B.161 | C.1 |
| 1.364 | B.162 | C.1 |
| 1.365 | B.163 | C.1 |
| 1.366 | B.164 | C.1 |
| 1.367 | B.165 | C.1 |
| 1.368 | B.166 | C.1 |
| 1.369 | B.167 | C.1 |
| 1.370 | B.168 | C.1 |
| 1.371 | B.169 | C.1 |
| 1.372 | B.170 | C.1 |
| 1.373 | B.171 | C.1 |
| 1.374 | B.172 | C.1 |
| 1.375 | B.173 | C.1 |
| 1.376 | B.174 | C.1 |
| 1.377 | B.175 | C.1 |
| 1.378 | B.176 | C.1 |
| 1.379 | B.177 | C.1 |
| 1.380 | B.178 | C.1 |
| 1.381 | B.179 | C.1 |
| 1.382 | B.180 | C.1 |
| 1.383 | B.181 | C.1 |
| 1.384 | B.182 | C.1 |
| 1.385 | B.183 | C.1 |
| 1.386 | B.184 | C.1 |
| 1.387 | B.185 | C.1 |
| 1.388 | B.186 | C.1 |
| 1.389 | B.187 | C.1 |
| 1.390 | B.188 | C.1 |
| 1.391 | B.189 | C.1 |
| 1.392 | B.190 | C.1 |
| 1.393 | B.191 | C.1 |
| 1.394 | B.192 | C.1 |
| 1.395 | B.193 | C.1 |
| 1.396 | B.194 | C.1 |
| 1.397 | B.195 | C.1 |
| 1.398 | B.196 | C.1 |
| 1.399 | B.197 | C.1 |
| 1.400 | B.198 | C.1 |
| 1.401 | B.199 | C.1 |
| 1.402 | B.200 | C.1 |
| 1.403 | B.201 | C.1 |
| 1.404 | B.202 | C.1 |
| 1.405 | B.1 | C.2 |
| 1.406 | B.2 | C.2 |
| 1.407 | B.3 | C.2 |
| 1.408 | B.4 | C.2 |
| 1.409 | B.5 | C.2 |
| 1.410 | B.6 | C.2 |
| 1.411 | B.7 | C.2 |
| 1.412 | B.8 | C.2 |
| 1.413 | B.9 | C.2 |
| 1.414 | B.10 | C.2 |
| 1.415 | B.11 | C.2 |
| 1.416 | B.12 | C.2 |
| 1.417 | B.13 | C.2 |
| 1.418 | B.14 | C.2 |
| 1.419 | B.15 | C.2 |
| 1.420 | B.16 | C.2 |
| 1.421 | B.17 | C.2 |
| 1.422 | B.18 | C.2 |
| 1.423 | B.19 | C.2 |
| 1.424 | B.20 | C.2 |
| 1.425 | B.21 | C.2 |
| 1.426 | B.22 | C.2 |
| 1.427 | B.23 | C.2 |
| 1.428 | B.24 | C.2 |
| 1.429 | B.25 | C.2 |
| 1.430 | B.26 | C.2 |
| 1.431 | B.27 | C.2 |
| 1.432 | B.28 | C.2 |
| 1.433 | B.29 | C.2 |
| 1.434 | B.30 | C.2 |
| 1.435 | B.31 | C.2 |
| 1.436 | B.32 | C.2 |
| 1.437 | B.33 | C.2 |
| 1.438 | B.34 | C.2 |
| 1.439 | B.35 | C.2 |
| 1.440 | B.36 | C.2 |
| 1.441 | B.37 | C.2 |
| 1.442 | B.38 | C.2 |
| 1.443 | B.39 | C.2 |
| 1.444 | B.40 | C.2 |
| 1.445 | B.41 | C.2 |
| 1.446 | B.42 | C.2 |
| 1.447 | B.43 | C.2 |
| 1.448 | B.44 | C.2 |
| 1.449 | B.45 | C.2 |
| 1.450 | B.46 | C.2 |
| 1.451 | B.47 | C.2 |
| 1.452 | B.48 | C.2 |
| 1.453 | B.49 | C.2 |
| 1.454 | B.50 | C.2 |
| 1.455 | B.51 | C.2 |
| 1.456 | B.52 | C.2 |
| 1.457 | B.53 | C.2 |
| 1.458 | B.54 | C.2 |
| 1.459 | B.55 | C.2 |
| 1.460 | B.56 | C.2 |
| 1.461 | B.57 | C.2 |
| 1.462 | B.58 | C.2 |
| 1.463 | B.59 | C.2 |
| 1.464 | B.60 | C.2 |
| 1.465 | B.61 | C.2 |
| 1.466 | B.62 | C.2 |
| 1.467 | B.63 | C.2 |
| 1.468 | B.64 | C.2 |
| 1.469 | B.65 | C.2 |
| 1.470 | B.66 | C.2 |
| 1.471 | B.67 | C.2 |
| 1.472 | B.68 | C.2 |
| 1.473 | B.69 | C.2 |
| 1.474 | B.70 | C.2 |
| 1.475 | B.71 | C.2 |
| 1.476 | B.72 | C.2 |
| 1.477 | B.73 | C.2 |
| 1.478 | B.74 | C.2 |
| 1.479 | B.75 | C.2 |
| 1.480 | B.76 | C.2 |
| 1.481 | B.77 | C.2 |
| 1.482 | B.78 | C.2 |
| 1.483 | B.79 | C.2 |
| 1.484 | B.80 | C.2 |
| 1.485 | B.81 | C.2 |
| 1.486 | B.82 | C.2 |
| 1.487 | B.83 | C.2 |
| 1.488 | B.84 | C.2 |
| 1.489 | B.85 | C.2 |
| 1.490 | B.86 | C.2 |
| 1.491 | B.87 | C.2 |
| 1.492 | B.88 | C.2 |
| 1.493 | B.89 | C.2 |
| 1.494 | B.90 | C.2 |
| 1.495 | B.91 | C.2 |
| 1.496 | B.92 | C.2 |
| 1.497 | B.93 | C.2 |
| 1.498 | B.94 | C.2 |
| 1.499 | B.95 | C.2 |
| 1.500 | B.96 | C.2 |
| 1.501 | B.97 | C.2 |
| 1.502 | B.98 | C.2 |
| 1.503 | B.99 | C.2 |
| 1.504 | B.100 | C.2 |
| 1.505 | B.101 | C.2 |
| 1.506 | B.102 | C.2 |
| 1.507 | B.103 | C.2 |
| 1.508 | B.104 | C.2 |
| 1.509 | B.105 | C.2 |
| 1.510 | B.106 | C.2 |
| 1.511 | B.107 | C.2 |
| 1.512 | B.108 | C.2 |
| 1.513 | B.109 | C.2 |
| 1.514 | B.110 | C.2 |
| 1.515 | B.111 | C.2 |
| 1.516 | B.112 | C.2 |
| 1.517 | B.113 | C.2 |
| 1.518 | B.114 | C.2 |
| 1.519 | B.115 | C.2 |
| 1.520 | B.116 | C.2 |
| 1.521 | B.117 | C.2 |
| 1.522 | B.118 | C.2 |
| 1.523 | B.119 | C.2 |
| 1.524 | B.120 | C.2 |
| 1.525 | B.121 | C.2 |
| 1.526 | B.122 | C.2 |
| 1.527 | B.123 | C.2 |
| 1.528 | B.124 | C.2 |
| 1.529 | B.125 | C.2 |
| 1.530 | B.126 | C.2 |
| 1.531 | B.127 | C.2 |
| 1.532 | B.128 | C.2 |
| 1.533 | B.129 | C.2 |
| 1.534 | B.130 | C.2 |
| 1.535 | B.131 | C.2 |
| 1.536 | B.132 | C.2 |
| 1.537 | B.133 | C.2 |
| 1.538 | B.134 | C.2 |
| 1.539 | B.135 | C.2 |
| 1.540 | B.136 | C.2 |
| 1.541 | B.137 | C.2 |
| 1.542 | B.138 | C.2 |
| 1.543 | B.139 | C.2 |
| 1.544 | B.140 | C.2 |
| 1.545 | B.141 | C.2 |
| 1.546 | B.142 | C.2 |
| 1.547 | B.143 | C.2 |
| 1.548 | B.144 | C.2 |
| 1.549 | B.145 | C.2 |
| 1.550 | B.146 | C.2 |
| 1.551 | B.147 | C.2 |
| 1.552 | B.148 | C.2 |
| 1.553 | B.149 | C.2 |
| 1.554 | B.150 | C.2 |
| 1.555 | B.151 | C.2 |
| 1.556 | B.152 | C.2 |
| 1.557 | B.153 | C.2 |
| 1.558 | B.154 | C.2 |
| 1.559 | B.155 | C.2 |
| 1.560 | B.156 | C.2 |
| 1.561 | B.157 | C.2 |
| 1.562 | B.158 | C.2 |
| 1.563 | B.159 | C.2 |
| 1.564 | B.160 | C.2 |
| 1.565 | B.161 | C.2 |
| 1.566 | B.162 | C.2 |
| 1.567 | B.163 | C.2 |
| 1.568 | B.164 | C.2 |
| 1.569 | B.165 | C.2 |
| 1.570 | B.166 | C.2 |
| 1.571 | B.167 | C.2 |
| 1.572 | B.168 | C.2 |
| 1.573 | B.169 | C.2 |
| 1.574 | B.170 | C.2 |
| 1.575 | B.171 | C.2 |
| 1.576 | B.172 | C.2 |
| 1.577 | B.173 | C.2 |
| 1.578 | B.174 | C.2 |
| 1.579 | B.175 | C.2 |
| 1.580 | B.176 | C.2 |
| 1.581 | B.177 | C.2 |
| 1.582 | B.178 | C.2 |
| 1.583 | B.179 | C.2 |
| 1.584 | B.180 | C.2 |
| 1.585 | B.181 | C.2 |
| 1.586 | B.182 | C.2 |
| 1.587 | B.183 | C.2 |
| 1.588 | B.184 | C.2 |
| 1.589 | B.185 | C.2 |
| 1.590 | B.186 | C.2 |
| 1.591 | B.187 | C.2 |
| 1.592 | B.188 | C.2 |
| 1.593 | B.189 | C.2 |
| 1.594 | B.190 | C.2 |
| 1.595 | B.191 | C.2 |
| 1.596 | B.192 | C.2 |
| 1.597 | B.193 | C.2 |
| 1.598 | B.194 | C.2 |
| 1.599 | B.195 | C.2 |
| 1.600 | B.196 | C.2 |
| 1.601 | B.197 | C.2 |
| 1.602 | B.198 | C.2 |
| 1.603 | B.199 | C.2 |
| 1.604 | B.200 | C.2 |
| 1.605 | B.201 | C.2 |
| 1.606 | B.202 | C.2 |
| 1.607 | B.1 | C.3 |
| 1.608 | B.2 | C.3 |
| 1.609 | B.3 | C.3 |
| 1.610 | B.4 | C.3 |
| 1.611 | B.5 | C.3 |
| 1.612 | B.6 | C.3 |
| 1.613 | B.7 | C.3 |
| 1.614 | B.8 | C.3 |
| 1.615 | B.9 | C.3 |
| 1.616 | B.10 | C.3 |
| 1.617 | B.11 | C.3 |
| 1.618 | B.12 | C.3 |
| 1.619 | B.13 | C.3 |
| 1.620 | B.14 | C.3 |
| 1.621 | B.15 | C.3 |
| 1.622 | B.16 | C.3 |
| 1.623 | B.17 | C.3 |
| 1.624 | B.18 | C.3 |
| 1.625 | B.19 | C.3 |
| 1.626 | B.20 | C.3 |
| 1.627 | B.21 | C.3 |
| 1.628 | B.22 | C.3 |
| 1.629 | B.23 | C.3 |
| 1.630 | B.24 | C.3 |
| 1.631 | B.25 | C.3 |
| 1.632 | B.26 | C.3 |
| 1.633 | B.27 | C.3 |
| 1.634 | B.28 | C.3 |
| 1.635 | B.29 | C.3 |
| 1.636 | B.30 | C.3 |
| 1.637 | B.31 | C.3 |
| 1.638 | B.32 | C.3 |
| 1.639 | B.33 | C.3 |
| 1.640 | B.34 | C.3 |
| 1.641 | B.35 | C.3 |
| 1.642 | B.36 | C.3 |
| 1.643 | B.37 | C.3 |
| 1.644 | B.38 | C.3 |
| 1.645 | B.39 | C.3 |
| 1.646 | B.40 | C.3 |
| 1.647 | B.41 | C.3 |
| 1.648 | B.42 | C.3 |
| 1.649 | B.43 | C.3 |
| 1.650 | B.44 | C.3 |
| 1.651 | B.45 | C.3 |
| 1.652 | B.46 | C.3 |
| 1.653 | B.47 | C.3 |
| 1.654 | B.48 | C.3 |
| 1.655 | B.49 | C.3 |
| 1.656 | B.50 | C.3 |
| 1.657 | B.51 | C.3 |
| 1.658 | B.52 | C.3 |
| 1.659 | B.53 | C.3 |
| 1.660 | B.54 | C.3 |
| 1.661 | B.55 | C.3 |
| 1.662 | B.56 | C.3 |
| 1.663 | B.57 | C.3 |
| 1.664 | B.58 | C.3 |
| 1.665 | B.59 | C.3 |
| 1.666 | B.60 | C.3 |
| 1.667 | B.61 | C.3 |
| 1.668 | B.62 | C.3 |
| 1.669 | B.63 | C.3 |
| 1.670 | B.64 | C.3 |
| 1.671 | B.65 | C.3 |
| 1.672 | B.66 | C.3 |
| 1.673 | B.67 | C.3 |
| 1.674 | B.68 | C.3 |
| 1.675 | B.69 | C.3 |
| 1.676 | B.70 | C.3 |
| 1.677 | B.71 | C.3 |
| 1.678 | B.72 | C.3 |
| 1.679 | B.73 | C.3 |
| 1.680 | B.74 | C.3 |
| 1.681 | B.75 | C.3 |
| 1.682 | B.76 | C.3 |
| 1.683 | B.77 | C.3 |
| 1.684 | B.78 | C.3 |
| 1.685 | B.79 | C.3 |
| 1.686 | B.80 | C.3 |
| 1.687 | B.81 | C.3 |
| 1.688 | B.82 | C.3 |
| 1.689 | B.83 | C.3 |
| 1.690 | B.84 | C.3 |
| 1.691 | B.85 | C.3 |
| 1.692 | B.86 | C.3 |
| 1.693 | B.87 | C.3 |
| 1.694 | B.88 | C.3 |
| 1.695 | B.89 | C.3 |
| 1.696 | B.90 | C.3 |
| 1.697 | B.91 | C.3 |
| 1.698 | B.92 | C.3 |
| 1.699 | B.93 | C.3 |
| 1.700 | B.94 | C.3 |
| 1.701 | B.95 | C.3 |
| 1.702 | B.96 | C.3 |
| 1.703 | B.97 | C.3 |
| 1.704 | B.98 | C.3 |
| 1.705 | B.99 | C.3 |
| 1.706 | B.100 | C.3 |
| 1.707 | B.101 | C.3 |
| 1.708 | B.102 | C.3 |
| 1.709 | B.103 | C.3 |
| 1.710 | B.104 | C.3 |
| 1.711 | B.105 | C.3 |
| 1.712 | B.106 | C.3 |
| 1.713 | B.107 | C.3 |
| 1.714 | B.108 | C.3 |
| 1.715 | B.109 | C.3 |
| 1.716 | B.110 | C.3 |
| 1.717 | B.111 | C.3 |
| 1.718 | B.112 | C.3 |
| 1.719 | B.113 | C.3 |
| 1.720 | B.114 | C.3 |
| 1.721 | B.115 | C.3 |
| 1.722 | B.116 | C.3 |
| 1.723 | B.117 | C.3 |
| 1.724 | B.118 | C.3 |
| 1.725 | B.119 | C.3 |
| 1.726 | B.120 | C.3 |
| 1.727 | B.121 | C.3 |
| 1.728 | B.122 | C.3 |
| 1.729 | B.123 | C.3 |
| 1.730 | B.124 | C.3 |
| 1.731 | B.125 | C.3 |
| 1.732 | B.126 | C.3 |
| 1.733 | B.127 | C.3 |
| 1.734 | B.128 | C.3 |
| 1.735 | B.129 | C.3 |
| 1.736 | B.130 | C.3 |
| 1.737 | B.131 | C.3 |
| 1.738 | B.132 | C.3 |
| 1.739 | B.133 | C.3 |
| 1.740 | B.134 | C.3 |
| 1.741 | B.135 | C.3 |
| 1.742 | B.136 | C.3 |
| 1.743 | B.137 | C.3 |
| 1.744 | B.138 | C.3 |
| 1.745 | B.139 | C.3 |
| 1.746 | B.140 | C.3 |
| 1.747 | B.141 | C.3 |
| 1.748 | B.142 | C.3 |
| 1.749 | B.143 | C.3 |
| 1.750 | B.144 | C.3 |
| 1.751 | B.145 | C.3 |
| 1.752 | B.146 | C.3 |
| 1.753 | B.147 | C.3 |
| 1.754 | B.148 | C.3 |
| 1.755 | B.149 | C.3 |
| 1.756 | B.150 | C.3 |
| 1.757 | B.151 | C.3 |
| 1.758 | B.152 | C.3 |
| 1.759 | B.153 | C.3 |
| 1.760 | B.154 | C.3 |
| 1.761 | B.155 | C.3 |
| 1.762 | B.156 | C.3 |
| 1.763 | B.157 | C.3 |
| 1.764 | B.158 | C.3 |
| 1.765 | B.159 | C.3 |
| 1.766 | B.160 | C.3 |
| 1.767 | B.161 | C.3 |
| 1.768 | B.162 | C.3 |
| 1.769 | B.163 | C.3 |
| 1.770 | B.164 | C.3 |
| 1.771 | B.165 | C.3 |
| 1.772 | B.166 | C.3 |
| 1.773 | B.167 | C.3 |
| 1.774 | B.168 | C.3 |
| 1.775 | B.169 | C.3 |
| 1.776 | B.170 | C.3 |
| 1.777 | B.171 | C.3 |
| 1.778 | B.172 | C.3 |
| 1.779 | B.173 | C.3 |
| 1.780 | B.174 | C.3 |
| 1.781 | B.175 | C.3 |
| 1.782 | B.176 | C.3 |
| 1.783 | B.177 | C.3 |
| 1.784 | B.178 | C.3 |
| 1.785 | B.179 | C.3 |
| 1.786 | B.180 | C.3 |
| 1.787 | B.181 | C.3 |
| 1.788 | B.182 | C.3 |
| 1.789 | B.183 | C.3 |
| 1.790 | B.184 | C.3 |
| 1.791 | B.185 | C.3 |
| 1.792 | B.186 | C.3 |
| 1.793 | B.187 | C.3 |
| 1.794 | B.188 | C.3 |
| 1.795 | B.189 | C.3 |
| 1.796 | B.190 | C.3 |
| 1.797 | B.191 | C.3 |
| 1.798 | B.192 | C.3 |
| 1.799 | B.193 | C.3 |
| 1.800 | B.194 | C.3 |
| 1.801 | B.195 | C.3 |
| 1.802 | B.196 | C.3 |
| 1.803 | B.197 | C.3 |
| 1.804 | B.198 | C.3 |
| 1.805 | B.199 | C.3 |
| 1.806 | B.200 | C.3 |
| 1.807 | B.201 | C.3 |
| 1.808 | B.202 | C.3 |
| 1.809 | B.1 | C.4 |
| 1.810 | B.2 | C.4 |
| 1.811 | B.3 | C.4 |
| 1.812 | B.4 | C.4 |
| 1.813 | B.5 | C.4 |
| 1.814 | B.6 | C.4 |
| 1.815 | B.7 | C.4 |
| 1.816 | B.8 | C.4 |
| 1.817 | B.9 | C.4 |
| 1.818 | B.10 | C.4 |
| 1.819 | B.11 | C.4 |
| 1.820 | B.12 | C.4 |
| 1.821 | B.13 | C.4 |
| 1.822 | B.14 | C.4 |
| 1.823 | B.15 | C.4 |
| 1.824 | B.16 | C.4 |
| 1.825 | B.17 | C.4 |
| 1.826 | B.18 | C.4 |
| 1.827 | B.19 | C.4 |
| 1.828 | B.20 | C.4 |
| 1.829 | B.21 | C.4 |
| 1.830 | B.22 | C.4 |
| 1.831 | B.23 | C.4 |
| 1.832 | B.24 | C.4 |
| 1.833 | B.25 | C.4 |
| 1.834 | B.26 | C.4 |
| 1.835 | B.27 | C.4 |
| 1.836 | B.28 | C.4 |
| 1.837 | B.29 | C.4 |
| 1.838 | B.30 | C.4 |
| 1.839 | B.31 | C.4 |
| 1.840 | B.32 | C.4 |
| 1.841 | B.33 | C.4 |
| 1.842 | B.34 | C.4 |
| 1.843 | B.35 | C.4 |
| 1.844 | B.36 | C.4 |
| 1.845 | B.37 | C.4 |
| 1.846 | B.38 | C.4 |
| 1.847 | B.39 | C.4 |
| 1.848 | B.40 | C.4 |
| 1.849 | B.41 | C.4 |
| 1.850 | B.42 | C.4 |
| 1.851 | B.43 | C.4 |
| 1.852 | B.44 | C.4 |
| 1.853 | B.45 | C.4 |
| 1.854 | B.46 | C.4 |
| 1.855 | B.47 | C.4 |
| 1.856 | B.48 | C.4 |
| 1.857 | B.49 | C.4 |
| 1.858 | B.50 | C.4 |
| 1.859 | B.51 | C.4 |
| 1.860 | B.52 | C.4 |
| 1.861 | B.53 | C.4 |
| 1.862 | B.54 | C.4 |
| 1.863 | B.55 | C.4 |
| 1.864 | B.56 | C.4 |
| 1.865 | B.57 | C.4 |
| 1.866 | B.58 | C.4 |
| 1.867 | B.59 | C.4 |
| 1.868 | B.60 | C.4 |
| 1.869 | B.61 | C.4 |
| 1.870 | B.62 | C.4 |
| 1.871 | B.63 | C.4 |
| 1.872 | B.64 | C.4 |
| 1.873 | B.65 | C.4 |
| 1.874 | B.66 | C.4 |
| 1.875 | B.67 | C.4 |
| 1.876 | B.68 | C.4 |
| 1.877 | B.69 | C.4 |
| 1.878 | B.70 | C.4 |
| 1.879 | B.71 | C.4 |
| 1.880 | B.72 | C.4 |
| 1.881 | B.73 | C.4 |
| 1.882 | B.74 | C.4 |
| 1.883 | B.75 | C.4 |
| 1.884 | B.76 | C.4 |
| 1.885 | B.77 | C.4 |
| 1.886 | B.78 | C.4 |
| 1.887 | B.79 | C.4 |
| 1.888 | B.80 | C.4 |
| 1.889 | B.81 | C.4 |
| 1.890 | B.82 | C.4 |
| 1.891 | B.83 | C.4 |
| 1.892 | B.84 | C.4 |
| 1.893 | B.85 | C.4 |
| 1.894 | B.86 | C.4 |
| 1.895 | B.87 | C.4 |
| 1.896 | B.88 | C.4 |
| 1.897 | B.89 | C.4 |
| 1.898 | B.90 | C.4 |
| 1.899 | B.91 | C.4 |
| 1.900 | B.92 | C.4 |
| 1.901 | B.93 | C.4 |
| 1.902 | B.94 | C.4 |
| 1.903 | B.95 | C.4 |
| 1.904 | B.96 | C.4 |
| 1.905 | B.97 | C.4 |
| 1.906 | B.98 | C.4 |
| 1.907 | B.99 | C.4 |
| 1.908 | B.100 | C.4 |
| 1.909 | B.101 | C.4 |
| 1.910 | B.102 | C.4 |
| 1.911 | B.103 | C.4 |
| 1.912 | B.104 | C.4 |
| 1.913 | B.105 | C.4 |
| 1.914 | B.106 | C.4 |
| 1.915 | B.107 | C.4 |
| 1.916 | B.108 | C.4 |
| 1.917 | B.109 | C.4 |
| 1.918 | B.110 | C.4 |
| 1.919 | B.111 | C.4 |
| 1.920 | B.112 | C.4 |
| 1.921 | B.113 | C.4 |
| 1.922 | B.114 | C.4 |
| 1.923 | B.115 | C.4 |
| 1.924 | B.116 | C.4 |
| 1.925 | B.117 | C.4 |
| 1.926 | B.118 | C.4 |
| 1.927 | B.119 | C.4 |
| 1.928 | B.120 | C.4 |
| 1.929 | B.121 | C.4 |
| 1.930 | B.122 | C.4 |
| 1.931 | B.123 | C.4 |
| 1.932 | B.124 | C.4 |
| 1.933 | B.125 | C.4 |
| 1.934 | B.126 | C.4 |
| 1.935 | B.127 | C.4 |
| 1.936 | B.128 | C.4 |
| 1.937 | B.129 | C.4 |
| 1.938 | B.130 | C.4 |
| 1.939 | B.131 | C.4 |
| 1.940 | B.132 | C.4 |
| 1.941 | B.133 | C.4 |
| 1.942 | B.134 | C.4 |
| 1.943 | B.135 | C.4 |
| 1.944 | B.136 | C.4 |
| 1.945 | B.137 | C.4 |
| 1.946 | B.138 | C.4 |
| 1.947 | B.139 | C.4 |
| 1.948 | B.140 | C.4 |
| 1.949 | B.141 | C.4 |
| 1.950 | B.142 | C.4 |
| 1.951 | B.143 | C.4 |
| 1.952 | B.144 | C.4 |
| 1.953 | B.145 | C.4 |
| 1.954 | B.146 | C.4 |
| 1.955 | B.147 | C.4 |
| 1.956 | B.148 | C.4 |
| 1.957 | B.149 | C.4 |
| 1.958 | B.150 | C.4 |
| 1.959 | B.151 | C.4 |
| 1.960 | B.152 | C.4 |
| 1.961 | B.153 | C.4 |
| 1.962 | B.154 | C.4 |
| 1.963 | B.155 | C.4 |
| 1.964 | B.156 | C.4 |
| 1.965 | B.157 | C.4 |
| 1.966 | B.158 | C.4 |
| 1.967 | B.159 | C.4 |
| 1.968 | B.160 | C.4 |
| 1.969 | B.161 | C.4 |
| 1.970 | B.162 | C.4 |
| 1.971 | B.163 | C.4 |
| 1.972 | B.164 | C.4 |
| 1.973 | B.165 | C.4 |
| 1.974 | B.166 | C.4 |
| 1.975 | B.167 | C.4 |
| 1.976 | B.168 | C.4 |
| 1.977 | B.169 | C.4 |
| 1.978 | B.170 | C.4 |
| 1.979 | B.171 | C.4 |
| 1.980 | B.172 | C.4 |
| 1.981 | B.173 | C.4 |
| 1.982 | B.174 | C.4 |
| 1.983 | B.175 | C.4 |
| 1.984 | B.176 | C.4 |
| 1.985 | B.177 | C.4 |
| 1.986 | B.178 | C.4 |
| 1.987 | B.179 | C.4 |
| 1.988 | B.180 | C.4 |
| 1.989 | B.181 | C.4 |
| 1.990 | B.182 | C.4 |
| 1.991 | B.183 | C.4 |
| 1.992 | B.184 | C.4 |
| 1.993 | B.185 | C.4 |
| 1.994 | B.186 | C.4 |
| 1.995 | B.187 | C.4 |
| 1.996 | B.188 | C.4 |
| 1.997 | B.189 | C.4 |
| 1.998 | B.190 | C.4 |
| 1.999 | B.191 | C.4 |
| 1.1000 | B.192 | C.4 |
| 1.1001 | B.193 | C.4 |
| 1.1002 | B.194 | C.4 |
| 1.1003 | B.195 | C.4 |
| 1.1004 | B.196 | C.4 |
| 1.1005 | B.197 | C.4 |
| 1.1006 | B.198 | C.4 |
| 1.1007 | B.199 | C.4 |
| 1.1008 | B.200 | C.4 |
| 1.1009 | B.201 | C.4 |
| 1.1010 | B.202 | C.4 |
| 1.1011 | B.1 | C.5 |
| 1.1012 | B.2 | C.5 |
| 1.1013 | B.3 | C.5 |
| 1.1014 | B.4 | C.5 |
| 1.1015 | B.5 | C.5 |
| 1.1016 | B.6 | C.5 |
| 1.1017 | B.7 | C.5 |
| 1.1018 | B.8 | C.5 |
| 1.1019 | B.9 | C.5 |
| 1.1020 | B.10 | C.5 |
| 1.1021 | B.11 | C.5 |
| 1.1022 | B.12 | C.5 |
| 1.1023 | B.13 | C.5 |
| 1.1024 | B.14 | C.5 |
| 1.1025 | B.15 | C.5 |
| 1.1026 | B.16 | C.5 |
| 1.1027 | B.17 | C.5 |
| 1.1028 | B.18 | C.5 |
| 1.1029 | B.19 | C.5 |
| 1.1030 | B.20 | C.5 |
| 1.1031 | B.21 | C.5 |
| 1.1032 | B.22 | C.5 |
| 1.1033 | B.23 | C.5 |
| 1.1034 | B.24 | C.5 |
| 1.1035 | B.25 | C.5 |
| 1.1036 | B.26 | C.5 |
| 1.1037 | B.27 | C.5 |
| 1.1038 | B.28 | C.5 |
| 1.1039 | B.29 | C.5 |
| 1.1040 | B.30 | C.5 |
| 1.1041 | B.31 | C.5 |
| 1.1042 | B.32 | C.5 |
| 1.1043 | B.33 | C.5 |
| 1.1044 | B.34 | C.5 |
| 1.1045 | B.35 | C.5 |
| 1.1046 | B.36 | C.5 |
| 1.1047 | B.37 | C.5 |
| 1.1048 | B.38 | C.5 |
| 1.1049 | B.39 | C.5 |
| 1.1050 | B.40 | C.5 |
| 1.1051 | B.41 | C.5 |
| 1.1052 | B.42 | C.5 |
| 1.1053 | B.43 | C.5 |
| 1.1054 | B.44 | C.5 |
| 1.1055 | B.45 | C.5 |
| 1.1056 | B.46 | C.5 |
| 1.1057 | B.47 | C.5 |
| 1.1058 | B.48 | C.5 |
| 1.1059 | B.49 | C.5 |
| 1.1060 | B.50 | C.5 |
| 1.1061 | B.51 | C.5 |
| 1.1062 | B.52 | C.5 |
| 1.1063 | B.53 | C.5 |
| 1.1064 | B.54 | C.5 |
| 1.1065 | B.55 | C.5 |
| 1.1066 | B.56 | C.5 |
| 1.1067 | B.57 | C.5 |
| 1.1068 | B.58 | C.5 |
| 1.1069 | B.59 | C.5 |
| 1.1070 | B.60 | C.5 |
| 1.1071 | B.61 | C.5 |
| 1.1072 | B.62 | C.5 |
| 1.1073 | B.63 | C.5 |
| 1.1074 | B.64 | C.5 |
| 1.1075 | B.65 | C.5 |
| 1.1076 | B.66 | C.5 |
| 1.1077 | B.67 | C.5 |
| 1.1078 | B.68 | C.5 |
| 1.1079 | B.69 | C.5 |
| 1.1080 | B.70 | C.5 |
| 1.1081 | B.71 | C.5 |
| 1.1082 | B.72 | C.5 |
| 1.1083 | B.73 | C.5 |
| 1.1084 | B.74 | C.5 |
| 1.1085 | B.75 | C.5 |
| 1.1086 | B.76 | C.5 |
| 1.1087 | B.77 | C.5 |
| 1.1088 | B.78 | C.5 |
| 1.1089 | B.79 | C.5 |
| 1.1090 | B.80 | C.5 |
| 1.1091 | B.81 | C.5 |
| 1.1092 | B.82 | C.5 |
| 1.1093 | B.83 | C.5 |
| 1.1094 | B.84 | C.5 |
| 1.1095 | B.85 | C.5 |
| 1.1096 | B.86 | C.5 |
| 1.1097 | B.87 | C.5 |
| 1.1098 | B.88 | C.5 |
| 1.1099 | B.89 | C.5 |
| 1.1100 | B.90 | C.5 |
| 1.1101 | B.91 | C.5 |
| 1.1102 | B.92 | C.5 |
| 1.1103 | B.93 | C.5 |
| 1.1104 | B.94 | C.5 |
| 1.1105 | B.95 | C.5 |
| 1.1106 | B.96 | C.5 |
| 1.1107 | B.97 | C.5 |
| 1.1108 | B.98 | C.5 |
| 1.1109 | B.99 | C.5 |
| 1.1110 | B.100 | C.5 |
| 1.1111 | B.101 | C.5 |
| 1.1112 | B.102 | C.5 |
| 1.1113 | B.103 | C.5 |
| 1.1114 | B.104 | C.5 |
| 1.1115 | B.105 | C.5 |
| 1.1116 | B.106 | C.5 |
| 1.1117 | B.107 | C.5 |
| 1.1118 | B.108 | C.5 |
| 1.1119 | B.109 | C.5 |
| 1.1120 | B.110 | C.5 |
| 1.1121 | B.111 | C.5 |
| 1.1122 | B.112 | C.5 |
| 1.1123 | B.113 | C.5 |
| 1.1124 | B.114 | C.5 |
| 1.1125 | B.115 | C.5 |
| 1.1126 | B.116 | C.5 |
| 1.1127 | B.117 | C.5 |
| 1.1128 | B.118 | C.5 |
| 1.1129 | B.119 | C.5 |
| 1.1130 | B.120 | C.5 |
| 1.1131 | B.121 | C.5 |
| 1.1132 | B.122 | C.5 |
| 1.1133 | B.123 | C.5 |
| 1.1134 | B.124 | C.5 |
| 1.1135 | B.125 | C.5 |
| 1.1136 | B.126 | C.5 |
| 1.1137 | B.127 | C.5 |
| 1.1138 | B.128 | C.5 |
| 1.1139 | B.129 | C.5 |
| 1.1140 | B.130 | C.5 |
| 1.1141 | B.131 | C.5 |
| 1.1142 | B.132 | C.5 |
| 1.1143 | B.133 | C.5 |
| 1.1144 | B.134 | C.5 |
| 1.1145 | B.135 | C.5 |
| 1.1146 | B.136 | C.5 |
| 1.1147 | B.137 | C.5 |
| 1.1148 | B.138 | C.5 |
| 1.1149 | B.139 | C.5 |
| 1.1150 | B.140 | C.5 |
| 1.1151 | B.141 | C.5 |
| 1.1152 | B.142 | C.5 |
| 1.1153 | B.143 | C.5 |
| 1.1154 | B.144 | C.5 |
| 1.1155 | B.145 | C.5 |
| 1.1156 | B.146 | C.5 |
| 1.1157 | B.147 | C.5 |
| 1.1158 | B.148 | C.5 |
| 1.1159 | B.149 | C.5 |
| 1.1160 | B.150 | C.5 |
| 1.1161 | B.151 | C.5 |
| 1.1162 | B.152 | C.5 |
| 1.1163 | B.153 | C.5 |
| 1.1164 | B.154 | C.5 |
| 1.1165 | B.155 | C.5 |
| 1.1166 | B.156 | C.5 |
| 1.1167 | B.157 | C.5 |
| 1.1168 | B.158 | C.5 |
| 1.1169 | B.159 | C.5 |
| 1.1170 | B.160 | C.5 |
| 1.1171 | B.161 | C.5 |
| 1.1172 | B.162 | C.5 |
| 1.1173 | B.163 | C.5 |
| 1.1174 | B.164 | C.5 |
| 1.1175 | B.165 | C.5 |
| 1.1176 | B.166 | C.5 |
| 1.1177 | B.167 | C.5 |
| 1.1178 | B.168 | C.5 |
| 1.1179 | B.169 | C.5 |
| 1.1180 | B.170 | C.5 |
| 1.1181 | B.171 | C.5 |
| 1.1182 | B.172 | C.5 |
| 1.1183 | B.173 | C.5 |
| 1.1184 | B.174 | C.5 |
| 1.1185 | B.175 | C.5 |
| 1.1186 | B.176 | C.5 |
| 1.1187 | B.177 | C.5 |
| 1.1188 | B.178 | C.5 |
| 1.1189 | B.179 | C.5 |
| 1.1190 | B.180 | C.5 |
| 1.1191 | B.181 | C.5 |
| 1.1192 | B.182 | C.5 |
| 1.1193 | B.183 | C.5 |
| 1.1194 | B.184 | C.5 |
| 1.1195 | B.185 | C.5 |
| 1.1196 | B.186 | C.5 |
| 1.1197 | B.187 | C.5 |
| 1.1198 | B.188 | C.5 |
| 1.1199 | B.189 | C.5 |
| 1.1200 | B.190 | C.5 |
| 1.1201 | B.191 | C.5 |
| 1.1202 | B.192 | C.5 |
| 1.1203 | B.193 | C.5 |
| 1.1204 | B.194 | C.5 |
| 1.1205 | B.195 | C.5 |
| 1.1206 | B.196 | C.5 |
| 1.1207 | B.197 | C.5 |
| 1.1208 | B.198 | C.5 |
| 1.1209 | B.199 | C.5 |
| 1.1210 | B.200 | C.5 |
| 1.1211 | B.201 | C.5 |
| 1.1212 | B.202 | C.5 |
| 1.1213 | B.1 | C.6 |
| 1.1214 | B.2 | C.6 |
| 1.1215 | B.3 | C.6 |
| 1.1216 | B.4 | C.6 |
| 1.1217 | B.5 | C.6 |
| 1.1218 | B.6 | C.6 |
| 1.1219 | B.7 | C.6 |
| 1.1220 | B.8 | C.6 |
| 1.1221 | B.9 | C.6 |
| 1.1222 | B.10 | C.6 |
| 1.1223 | B.11 | C.6 |
| 1.1224 | B.12 | C.6 |
| 1.1225 | B.13 | C.6 |
| 1.1226 | B.14 | C.6 |
| 1.1227 | B.15 | C.6 |
| 1.1228 | B.16 | C.6 |
| 1.1229 | B.17 | C.6 |
| 1.1230 | B.18 | C.6 |
| 1.1231 | B.19 | C.6 |
| 1.1232 | B.20 | C.6 |
| 1.1233 | B.21 | C.6 |
| 1.1234 | B.22 | C.6 |
| 1.1235 | B.23 | C.6 |
| 1.1236 | B.24 | C.6 |
| 1.1237 | B.25 | C.6 |
| 1.1238 | B.26 | C.6 |
| 1.1239 | B.27 | C.6 |
| 1.1240 | B.28 | C.6 |
| 1.1241 | B.29 | C.6 |
| 1.1242 | B.30 | C.6 |
| 1.1243 | B.31 | C.6 |
| 1.1244 | B.32 | C.6 |
| 1.1245 | B.33 | C.6 |
| 1.1246 | B.34 | C.6 |
| 1.1247 | B.35 | C.6 |
| 1.1248 | B.36 | C.6 |
| 1.1249 | B.37 | C.6 |
| 1.1250 | B.38 | C.6 |
| 1.1251 | B.39 | C.6 |
| 1.1252 | B.40 | C.6 |
| 1.1253 | B.41 | C.6 |
| 1.1254 | B.42 | C.6 |
| 1.1255 | B.43 | C.6 |
| 1.1256 | B.44 | C.6 |
| 1.1257 | B.45 | C.6 |
| 1.1258 | B.46 | C.6 |
| 1.1259 | B.47 | C.6 |
| 1.1260 | B.48 | C.6 |
| 1.1261 | B.49 | C.6 |
| 1.1262 | B.50 | C.6 |
| 1.1263 | B.51 | C.6 |
| 1.1264 | B.52 | C.6 |
| 1.1265 | B.53 | C.6 |
| 1.1266 | B.54 | C.6 |
| 1.1267 | B.55 | C.6 |
| 1.1268 | B.56 | C.6 |
| 1.1269 | B.57 | C.6 |
| 1.1270 | B.58 | C.6 |
| 1.1271 | B.59 | C.6 |
| 1.1272 | B.60 | C.6 |
| 1.1273 | B.61 | C.6 |
| 1.1274 | B.62 | C.6 |
| 1.1275 | B.63 | C.6 |
| 1.1276 | B.64 | C.6 |
| 1.1277 | B.65 | C.6 |
| 1.1278 | B.66 | C.6 |
| 1.1279 | B.67 | C.6 |
| 1.1280 | B.68 | C.6 |
| 1.1281 | B.69 | C.6 |
| 1.1282 | B.70 | C.6 |
| 1.1283 | B.71 | C.6 |
| 1.1284 | B.72 | C.6 |
| 1.1285 | B.73 | C.6 |
| 1.1286 | B.74 | C.6 |
| 1.1287 | B.75 | C.6 |
| 1.1288 | B.76 | C.6 |
| 1.1289 | B.77 | C.6 |
| 1.1290 | B.78 | C.6 |
| 1.1291 | B.79 | C.6 |
| 1.1292 | B.80 | C.6 |
| 1.1293 | B.81 | C.6 |
| 1.1294 | B.82 | C.6 |
| 1.1295 | B.83 | C.6 |
| 1.1296 | B.84 | C.6 |
| 1.1297 | B.85 | C.6 |
| 1.1298 | B.86 | C.6 |
| 1.1299 | B.87 | C.6 |
| 1.1300 | B.88 | C.6 |
| 1.1301 | B.89 | C.6 |
| 1.1302 | B.90 | C.6 |
| 1.1303 | B.91 | C.6 |
| 1.1304 | B.92 | C.6 |
| 1.1305 | B.93 | C.6 |
| 1.1306 | B.94 | C.6 |
| 1.1307 | B.95 | C.6 |
| 1.1308 | B.96 | C.6 |
| 1.1309 | B.97 | C.6 |
| 1.1310 | B.98 | C.6 |
| 1.1311 | B.99 | C.6 |
| 1.1312 | B.100 | C.6 |
| 1.1313 | B.101 | C.6 |
| 1.1314 | B.102 | C.6 |
| 1.1315 | B.103 | C.6 |
| 1.1316 | B.104 | C.6 |
| 1.1317 | B.105 | C.6 |
| 1.1318 | B.106 | C.6 |
| 1.1319 | B.107 | C.6 |
| 1.1320 | B.108 | C.6 |
| 1.1321 | B.109 | C.6 |
| 1.1322 | B.110 | C.6 |
| 1.1323 | B.111 | C.6 |
| 1.1324 | B.112 | C.6 |
| 1.1325 | B.113 | C.6 |
| 1.1326 | B.114 | C.6 |
| 1.1327 | B.115 | C.6 |
| 1.1328 | B.116 | C.6 |
| 1.1329 | B.117 | C.6 |
| 1.1330 | B.118 | C.6 |
| 1.1331 | B.119 | C.6 |
| 1.1332 | B.120 | C.6 |
| 1.1333 | B.121 | C.6 |
| 1.1334 | B.122 | C.6 |
| 1.1335 | B.123 | C.6 |
| 1.1336 | B.124 | C.6 |
| 1.1337 | B.125 | C.6 |
| 1.1338 | B.126 | C.6 |
| 1.1339 | B.127 | C.6 |
| 1.1340 | B.128 | C.6 |
| 1.1341 | B.129 | C.6 |
| 1.1342 | B.130 | C.6 |
| 1.1343 | B.131 | C.6 |
| 1.1344 | B.132 | C.6 |
| 1.1345 | B.133 | C.6 |
| 1.1346 | B.134 | C.6 |
| 1.1347 | B.135 | C.6 |
| 1.1348 | B.136 | C.6 |
| 1.1349 | B.137 | C.6 |
| 1.1350 | B.138 | C.6 |
| 1.1351 | B.139 | C.6 |
| 1.1352 | B.140 | C.6 |
| 1.1353 | B.141 | C.6 |
| 1.1354 | B.142 | C.6 |
| 1.1355 | B.143 | C.6 |
| 1.1356 | B.144 | C.6 |
| 1.1357 | B.145 | C.6 |
| 1.1358 | B.146 | C.6 |
| 1.1359 | B.147 | C.6 |
| 1.1360 | B.148 | C.6 |
| 1.1361 | B.149 | C.6 |
| 1.1362 | B.150 | C.6 |
| 1.1363 | B.151 | C.6 |
| 1.1364 | B.152 | C.6 |
| 1.1365 | B.153 | C.6 |
| 1.1366 | B.154 | C.6 |
| 1.1367 | B.155 | C.6 |
| 1.1368 | B.156 | C.6 |
| 1.1369 | B.157 | C.6 |
| 1.1370 | B.158 | C.6 |
| 1.1371 | B.159 | C.6 |
| 1.1372 | B.160 | C.6 |
| 1.1373 | B.161 | C.6 |
| 1.1374 | B.162 | C.6 |
| 1.1375 | B.163 | C.6 |
| 1.1376 | B.164 | C.6 |
| 1.1377 | B.165 | C.6 |
| 1.1378 | B.166 | C.6 |
| 1.1379 | B.167 | C.6 |
| 1.1380 | B.168 | C.6 |
| 1.1381 | B.169 | C.6 |
| 1.1382 | B.170 | C.6 |
| 1.1383 | B.171 | C.6 |
| 1.1384 | B.172 | C.6 |
| 1.1385 | B.173 | C.6 |
| 1.1386 | B.174 | C.6 |
| 1.1387 | B.175 | C.6 |
| 1.1388 | B.176 | C.6 |
| 1.1389 | B.177 | C.6 |
| 1.1390 | B.178 | C.6 |
| 1.1391 | B.179 | C.6 |
| 1.1392 | B.180 | C.6 |
| 1.1393 | B.181 | C.6 |
| 1.1394 | B.182 | C.6 |
| 1.1395 | B.183 | C.6 |
| 1.1396 | B.184 | C.6 |
| 1.1397 | B.185 | C.6 |
| 1.1398 | B.186 | C.6 |
| 1.1399 | B.187 | C.6 |
| 1.1400 | B.188 | C.6 |
| 1.1401 | B.189 | C.6 |
| 1.1402 | B.190 | C.6 |
| 1.1403 | B.191 | C.6 |
| 1.1404 | B.192 | C.6 |
| 1.1405 | B.193 | C.6 |
| 1.1406 | B.194 | C.6 |
| 1.1407 | B.195 | C.6 |
| 1.1408 | B.196 | C.6 |
| 1.1409 | B.197 | C.6 |
| 1.1410 | B.198 | C.6 |
| 1.1411 | B.199 | C.6 |
| 1.1412 | B.200 | C.6 |
| 1.1413 | B.201 | C.6 |
| 1.1414 | B.202 | C.6 |
| 1.1415 | B.1 | C.7 |
| 1.1416 | B.2 | C.7 |
| 1.1417 | B.3 | C.7 |
| 1.1418 | B.4 | C.7 |
| 1.1419 | B.5 | C.7 |
| 1.1420 | B.6 | C.7 |
| 1.1421 | B.7 | C.7 |
| 1.1422 | B.8 | C.7 |
| 1.1423 | B.9 | C.7 |
| 1.1424 | B.10 | C.7 |
| 1.1425 | B.11 | C.7 |
| 1.1426 | B.12 | C.7 |
| 1.1427 | B.13 | C.7 |
| 1.1428 | B.14 | C.7 |
| 1.1429 | B.15 | C.7 |
| 1.1430 | B.16 | C.7 |
| 1.1431 | B.17 | C.7 |
| 1.1432 | B.18 | C.7 |
| 1.1433 | B.19 | C.7 |
| 1.1434 | B.20 | C.7 |
| 1.1435 | B.21 | C.7 |
| 1.1436 | B.22 | C.7 |
| 1.1437 | B.23 | C.7 |
| 1.1438 | B.24 | C.7 |
| 1.1439 | B.25 | C.7 |
| 1.1440 | B.26 | C.7 |
| 1.1441 | B.27 | C.7 |
| 1.1442 | B.28 | C.7 |
| 1.1443 | B.29 | C.7 |
| 1.1444 | B.30 | C.7 |
| 1.1445 | B.31 | C.7 |
| 1.1446 | B.32 | C.7 |
| 1.1447 | B.33 | C.7 |
| 1.1448 | B.34 | C.7 |
| 1.1449 | B.35 | C.7 |
| 1.1450 | B.36 | C.7 |
| 1.1451 | B.37 | C.7 |
| 1.1452 | B.38 | C.7 |
| 1.1453 | B.39 | C.7 |
| 1.1454 | B.40 | C.7 |
| 1.1455 | B.41 | C.7 |
| 1.1456 | B.42 | C.7 |
| 1.1457 | B.43 | C.7 |
| 1.1458 | B.44 | C.7 |
| 1.1459 | B.45 | C.7 |
| 1.1460 | B.46 | C.7 |
| 1.1461 | B.47 | C.7 |
| 1.1462 | B.48 | C.7 |
| 1.1463 | B.49 | C.7 |
| 1.1464 | B.50 | C.7 |
| 1.1465 | B.51 | C.7 |
| 1.1466 | B.52 | C.7 |
| 1.1467 | B.53 | C.7 |
| 1.1468 | B.54 | C.7 |
| 1.1469 | B.55 | C.7 |
| 1.1470 | B.56 | C.7 |
| 1.1471 | B.57 | C.7 |
| 1.1472 | B.58 | C.7 |
| 1.1473 | B.59 | C.7 |
| 1.1474 | B.60 | C.7 |
| 1.1475 | B.61 | C.7 |
| 1.1476 | B.62 | C.7 |
| 1.1477 | B.63 | C.7 |
| 1.1478 | B.64 | C.7 |
| 1.1479 | B.65 | C.7 |
| 1.1480 | B.66 | C.7 |
| 1.1481 | B.67 | C.7 |
| 1.1482 | B.68 | C.7 |
| 1.1483 | B.69 | C.7 |
| 1.1484 | B.70 | C.7 |
| 1.1485 | B.71 | C.7 |
| 1.1486 | B.72 | C.7 |
| 1.1487 | B.73 | C.7 |
| 1.1488 | B.74 | C.7 |
| 1.1489 | B.75 | C.7 |
| 1.1490 | B.76 | C.7 |
| 1.1491 | B.77 | C.7 |
| 1.1492 | B.78 | C.7 |
| 1.1493 | B.79 | C.7 |
| 1.1494 | B.80 | C.7 |
| 1.1495 | B.81 | C.7 |
| 1.1496 | B.82 | C.7 |
| 1.1497 | B.83 | C.7 |
| 1.1498 | B.84 | C.7 |
| 1.1499 | B.85 | C.7 |
| 1.1500 | B.86 | C.7 |
| 1.1501 | B.87 | C.7 |
| 1.1502 | B.88 | C.7 |
| 1.1503 | B.89 | C.7 |
| 1.1504 | B.90 | C.7 |
| 1.1505 | B.91 | C.7 |
| 1.1506 | B.92 | C.7 |
| 1.1507 | B.93 | C.7 |
| 1.1508 | B.94 | C.7 |
| 1.1509 | B.95 | C.7 |
| 1.1510 | B.96 | C.7 |
| 1.1511 | B.97 | C.7 |
| 1.1512 | B.98 | C.7 |
| 1.1513 | B.99 | C.7 |
| 1.1514 | B.100 | C.7 |
| 1.1515 | B.101 | C.7 |
| 1.1516 | B.102 | C.7 |
| 1.1517 | B.103 | C.7 |
| 1.1518 | B.104 | C.7 |
| 1.1519 | B.105 | C.7 |
| 1.1520 | B.106 | C.7 |
| 1.1521 | B.107 | C.7 |
| 1.1522 | B.108 | C.7 |
| 1.1523 | B.109 | C.7 |
| 1.1524 | B.110 | C.7 |
| 1.1525 | B.111 | C.7 |
| 1.1526 | B.112 | C.7 |
| 1.1527 | B.113 | C.7 |
| 1.1528 | B.114 | C.7 |
| 1.1529 | B.115 | C.7 |
| 1.1530 | B.116 | C.7 |
| 1.1531 | B.117 | C.7 |
| 1.1532 | B.118 | C.7 |
| 1.1533 | B.119 | C.7 |
| 1.1534 | B.120 | C.7 |
| 1.1535 | B.121 | C.7 |
| 1.1536 | B.122 | C.7 |
| 1.1537 | B.123 | C.7 |
| 1.1538 | B.124 | C.7 |
| 1.1539 | B.125 | C.7 |
| 1.1540 | B.126 | C.7 |
| 1.1541 | B.127 | C.7 |
| 1.1542 | B.128 | C.7 |
| 1.1543 | B.129 | C.7 |
| 1.1544 | B.130 | C.7 |
| 1.1545 | B.131 | C.7 |
| 1.1546 | B.132 | C.7 |
| 1.1547 | B.133 | C.7 |
| 1.1548 | B.134 | C.7 |
| 1.1549 | B.135 | C.7 |
| 1.1550 | B.136 | C.7 |
| 1.1551 | B.137 | C.7 |
| 1.1552 | B.138 | C.7 |
| 1.1553 | B.139 | C.7 |
| 1.1554 | B.140 | C.7 |
| 1.1555 | B.141 | C.7 |
| 1.1556 | B.142 | C.7 |
| 1.1557 | B.143 | C.7 |
| 1.1558 | B.144 | C.7 |
| 1.1559 | B.145 | C.7 |
| 1.1560 | B.146 | C.7 |
| 1.1561 | B.147 | C.7 |
| 1.1562 | B.148 | C.7 |
| 1.1563 | B.149 | C.7 |
| 1.1564 | B.150 | C.7 |
| 1.1565 | B.151 | C.7 |
| 1.1566 | B.152 | C.7 |
| 1.1567 | B.153 | C.7 |
| 1.1568 | B.154 | C.7 |
| 1.1569 | B.155 | C.7 |
| 1.1570 | B.156 | C.7 |
| 1.1571 | B.157 | C.7 |
| 1.1572 | B.158 | C.7 |
| 1.1573 | B.159 | C.7 |
| 1.1574 | B.160 | C.7 |
| 1.1575 | B.161 | C.7 |
| 1.1576 | B.162 | C.7 |
| 1.1577 | B.163 | C.7 |
| 1.1578 | B.164 | C.7 |
| 1.1579 | B.165 | C.7 |
| 1.1580 | B.166 | C.7 |
| 1.1581 | B.167 | C.7 |
| 1.1582 | B.168 | C.7 |
| 1.1583 | B.169 | C.7 |
| 1.1584 | B.170 | C.7 |
| 1.1585 | B.171 | C.7 |
| 1.1586 | B.172 | C.7 |
| 1.1587 | B.173 | C.7 |
| 1.1588 | B.174 | C.7 |
| 1.1589 | B.175 | C.7 |
| 1.1590 | B.176 | C.7 |
| 1.1591 | B.177 | C.7 |
| 1.1592 | B.178 | C.7 |
| 1.1593 | B.179 | C.7 |
| 1.1594 | B.180 | C.7 |
| 1.1595 | B.181 | C.7 |
| 1.1596 | B.182 | C.7 |
| 1.1597 | B.183 | C.7 |
| 1.1598 | B.184 | C.7 |
| 1.1599 | B.185 | C.7 |
| 1.1600 | B.186 | C.7 |
| 1.1601 | B.187 | C.7 |
| 1.1602 | B.188 | C.7 |
| 1.1603 | B.189 | C.7 |
| 1.1604 | B.190 | C.7 |
| 1.1605 | B.191 | C.7 |
| 1.1606 | B.192 | C.7 |
| 1.1607 | B.193 | C.7 |
| 1.1608 | B.194 | C.7 |
| 1.1609 | B.195 | C.7 |
| 1.1610 | B.196 | C.7 |
| 1.1611 | B.197 | C.7 |
| 1.1612 | B.198 | C.7 |
| 1.1613 | B.199 | C.7 |
| 1.1614 | B.200 | C.7 |
| 1.1615 | B.201 | C.7 |
| 1.1616 | B.202 | C.7 |
| 1.1617 | B.1 | C.8 |
| 1.1618 | B.2 | C.8 |
| 1.1619 | B.3 | C.8 |
| 1.1620 | B.4 | C.8 |
| 1.1621 | B.5 | C.8 |
| 1.1622 | B.6 | C.8 |
| 1.1623 | B.7 | C.8 |
| 1.1624 | B.8 | C.8 |
| 1.1625 | B.9 | C.8 |
| 1.1626 | B.10 | C.8 |
| 1.1627 | B.11 | C.8 |
| 1.1628 | B.12 | C.8 |
| 1.1629 | B.13 | C.8 |
| 1.1630 | B.14 | C.8 |
| 1.1631 | B.15 | C.8 |
| 1.1632 | B.16 | C.8 |
| 1.1633 | B.17 | C.8 |
| 1.1634 | B.18 | C.8 |
| 1.1635 | B.19 | C.8 |
| 1.1636 | B.20 | C.8 |
| 1.1637 | B.21 | C.8 |
| 1.1638 | B.22 | C.8 |
| 1.1639 | B.23 | C.8 |
| 1.1640 | B.24 | C.8 |
| 1.1641 | B.25 | C.8 |
| 1.1642 | B.26 | C.8 |
| 1.1643 | B.27 | C.8 |
| 1.1644 | B.28 | C.8 |
| 1.1645 | B.29 | C.8 |
| 1.1646 | B.30 | C.8 |
| 1.1647 | B.31 | C.8 |
| 1.1648 | B.32 | C.8 |
| 1.1649 | B.33 | C.8 |
| 1.1650 | B.34 | C.8 |
| 1.1651 | B.35 | C.8 |
| 1.1652 | B.36 | C.8 |
| 1.1653 | B.37 | C.8 |
| 1.1654 | B.38 | C.8 |
| 1.1655 | B.39 | C.8 |
| 1.1656 | B.40 | C.8 |
| 1.1657 | B.41 | C.8 |
| 1.1658 | B.42 | C.8 |
| 1.1659 | B.43 | C.8 |
| 1.1660 | B.44 | C.8 |
| 1.1661 | B.45 | C.8 |
| 1.1662 | B.46 | C.8 |
| 1.1663 | B.47 | C.8 |
| 1.1664 | B.48 | C.8 |
| 1.1665 | B.49 | C.8 |
| 1.1666 | B.50 | C.8 |
| 1.1667 | B.51 | C.8 |
| 1.1668 | B.52 | C.8 |
| 1.1669 | B.53 | C.8 |
| 1.1670 | B.54 | C.8 |
| 1.1671 | B.55 | C.8 |
| 1.1672 | B.56 | C.8 |
| 1.1673 | B.57 | C.8 |
| 1.1674 | B.58 | C.8 |
| 1.1675 | B.59 | C.8 |
| 1.1676 | B.60 | C.8 |
| 1.1677 | B.61 | C.8 |
| 1.1678 | B.62 | C.8 |
| 1.1679 | B.63 | C.8 |
| 1.1680 | B.64 | C.8 |
| 1.1681 | B.65 | C.8 |
| 1.1682 | B.66 | C.8 |
| 1.1683 | B.67 | C.8 |
| 1.1684 | B.68 | C.8 |
| 1.1685 | B.69 | C.8 |
| 1.1686 | B.70 | C.8 |
| 1.1687 | B.71 | C.8 |
| 1.1688 | B.72 | C.8 |
| 1.1689 | B.73 | C.8 |
| 1.1690 | B.74 | C.8 |
| 1.1691 | B.75 | C.8 |
| 1.1692 | B.76 | C.8 |
| 1.1693 | B.77 | C.8 |
| 1.1694 | B.78 | C.8 |
| 1.1695 | B.79 | C.8 |
| 1.1696 | B.80 | C.8 |
| 1.1697 | B.81 | C.8 |
| 1.1698 | B.82 | C.8 |
| 1.1699 | B.83 | C.8 |
| 1.1700 | B.84 | C.8 |
| 1.1701 | B.85 | C.8 |
| 1.1702 | B.86 | C.8 |
| 1.1703 | B.87 | C.8 |
| 1.1704 | B.88 | C.8 |
| 1.1705 | B.89 | C.8 |
| 1.1706 | B.90 | C.8 |
| 1.1707 | B.91 | C.8 |
| 1.1708 | B.92 | C.8 |
| 1.1709 | B.93 | C.8 |
| 1.1710 | B.94 | C.8 |
| 1.1711 | B.95 | C.8 |
| 1.1712 | B.96 | C.8 |
| 1.1713 | B.97 | C.8 |
| 1.1714 | B.98 | C.8 |
| 1.1715 | B.99 | C.8 |
| 1.1716 | B.100 | C.8 |
| 1.1717 | B.101 | C.8 |
| 1.1718 | B.102 | C.8 |
| 1.1719 | B.103 | C.8 |
| 1.1720 | B.104 | C.8 |
| 1.1721 | B.105 | C.8 |
| 1.1722 | B.106 | C.8 |
| 1.1723 | B.107 | C.8 |
| 1.1724 | B.108 | C.8 |
| 1.1725 | B.109 | C.8 |
| 1.1726 | B.110 | C.8 |
| 1.1727 | B.111 | C.8 |
| 1.1728 | B.112 | C.8 |
| 1.1729 | B.113 | C.8 |
| 1.1730 | B.114 | C.8 |
| 1.1731 | B.115 | C.8 |
| 1.1732 | B.116 | C.8 |
| 1.1733 | B.117 | C.8 |
| 1.1734 | B.118 | C.8 |
| 1.1735 | B.119 | C.8 |
| 1.1736 | B.120 | C.8 |
| 1.1737 | B.121 | C.8 |
| 1.1738 | B.122 | C.8 |
| 1.1739 | B.123 | C.8 |
| 1.1740 | B.124 | C.8 |
| 1.1741 | B.125 | C.8 |
| 1.1742 | B.126 | C.8 |
| 1.1743 | B.127 | C.8 |
| 1.1744 | B.128 | C.8 |
| 1.1745 | B.129 | C.8 |
| 1.1746 | B.130 | C.8 |
| 1.1747 | B.131 | C.8 |
| 1.1748 | B.132 | C.8 |
| 1.1749 | B.133 | C.8 |
| 1.1750 | B.134 | C.8 |
| 1.1751 | B.135 | C.8 |
| 1.1752 | B.136 | C.8 |
| 1.1753 | B.137 | C.8 |
| 1.1754 | B.138 | C.8 |
| 1.1755 | B.139 | C.8 |
| 1.1756 | B.140 | C.8 |
| 1.1757 | B.141 | C.8 |
| 1.1758 | B.142 | C.8 |
| 1.1759 | B.143 | C.8 |
| 1.1760 | B.144 | C.8 |
| 1.1761 | B.145 | C.8 |
| 1.1762 | B.146 | C.8 |
| 1.1763 | B.147 | C.8 |
| 1.1764 | B.148 | C.8 |
| 1.1765 | B.149 | C.8 |
| 1.1766 | B.150 | C.8 |
| 1.1767 | B.151 | C.8 |
| 1.1768 | B.152 | C.8 |
| 1.1769 | B.153 | C.8 |
| 1.1770 | B.154 | C.8 |
| 1.1771 | B.155 | C.8 |
| 1.1772 | B.156 | C.8 |
| 1.1773 | B.157 | C.8 |
| 1.1774 | B.158 | C.8 |
| 1.1775 | B.159 | C.8 |
| 1.1776 | B.160 | C.8 |
| 1.1777 | B.161 | C.8 |
| 1.1778 | B.162 | C.8 |
| 1.1779 | B.163 | C.8 |
| 1.1780 | B.164 | C.8 |
| 1.1781 | B.165 | C.8 |
| 1.1782 | B.166 | C.8 |
| 1.1783 | B.167 | C.8 |
| 1.1784 | B.168 | C.8 |
| 1.1785 | B.169 | C.8 |
| 1.1786 | B.170 | C.8 |
| 1.1787 | B.171 | C.8 |
| 1.1788 | B.172 | C.8 |
| 1.1789 | B.173 | C.8 |
| 1.1790 | B.174 | C.8 |
| 1.1791 | B.175 | C.8 |
| 1.1792 | B.176 | C.8 |
| 1.1793 | B.177 | C.8 |
| 1.1794 | B.178 | C.8 |
| 1.1795 | B.179 | C.8 |
| 1.1796 | B.180 | C.8 |
| 1.1797 | B.181 | C.8 |
| 1.1798 | B.182 | C.8 |
| 1.1799 | B.183 | C.8 |
| 1.1800 | B.184 | C.8 |
| 1.1801 | B.185 | C.8 |
| 1.1802 | B.186 | C.8 |
| 1.1803 | B.187 | C.8 |
| 1.1804 | B.188 | C.8 |
| 1.1805 | B.189 | C.8 |
| 1.1806 | B.190 | C.8 |
| 1.1807 | B.191 | C.8 |
| 1.1808 | B.192 | C.8 |
| 1.1809 | B.193 | C.8 |
| 1.1810 | B.194 | C.8 |
| 1.1811 | B.195 | C.8 |
| 1.1812 | B.196 | C.8 |
| 1.1813 | B.197 | C.8 |
| 1.1814 | B.198 | C.8 |
| 1.1815 | B.199 | C.8 |
| 1.1816 | B.200 | C.8 |
| 1.1817 | B.201 | C.8 |
| 1.1818 | B.202 | C.8 |
| 1.1819 | B.1 | C.9 |
| 1.1820 | B.2 | C.9 |
| 1.1821 | B.3 | C.9 |
| 1.1822 | B.4 | C.9 |
| 1.1823 | B.5 | C.9 |
| 1.1824 | B.6 | C.9 |
| 1.1825 | B.7 | C.9 |
| 1.1826 | B.8 | C.9 |
| 1.1827 | B.9 | C.9 |
| 1.1828 | B.10 | C.9 |
| 1.1829 | B.11 | C.9 |
| 1.1830 | B.12 | C.9 |
| 1.1831 | B.13 | C.9 |
| 1.1832 | B.14 | C.9 |
| 1.1833 | B.15 | C.9 |
| 1.1834 | B.16 | C.9 |
| 1.1835 | B.17 | C.9 |
| 1.1836 | B.18 | C.9 |
| 1.1837 | B.19 | C.9 |
| 1.1838 | B.20 | C.9 |
| 1.1839 | B.21 | C.9 |
| 1.1840 | B.22 | C.9 |
| 1.1841 | B.23 | C.9 |
| 1.1842 | B.24 | C.9 |
| 1.1843 | B.25 | C.9 |
| 1.1844 | B.26 | C.9 |
| 1.1845 | B.27 | C.9 |
| 1.1846 | B.28 | C.9 |
| 1.1847 | B.29 | C.9 |
| 1.1848 | B.30 | C.9 |
| 1.1849 | B.31 | C.9 |
| 1.1850 | B.32 | C.9 |
| 1.1851 | B.33 | C.9 |
| 1.1852 | B.34 | C.9 |
| 1.1853 | B.35 | C.9 |
| 1.1854 | B.36 | C.9 |
| 1.1855 | B.37 | C.9 |
| 1.1856 | B.38 | C.9 |
| 1.1857 | B.39 | C.9 |
| 1.1858 | B.40 | C.9 |
| 1.1859 | B.41 | C.9 |
| 1.1860 | B.42 | C.9 |
| 1.1861 | B.43 | C.9 |
| 1.1862 | B.44 | C.9 |
| 1.1863 | B.45 | C.9 |
| 1.1864 | B.46 | C.9 |
| 1.1865 | B.47 | C.9 |
| 1.1866 | B.48 | C.9 |
| 1.1867 | B.49 | C.9 |
| 1.1868 | B.50 | C.9 |
| 1.1869 | B.51 | C.9 |
| 1.1870 | B.52 | C.9 |
| 1.1871 | B.53 | C.9 |
| 1.1872 | B.54 | C.9 |
| 1.1873 | B.55 | C.9 |
| 1.1874 | B.56 | C.9 |
| 1.1875 | B.57 | C.9 |
| 1.1876 | B.58 | C.9 |
| 1.1877 | B.59 | C.9 |
| 1.1878 | B.60 | C.9 |
| 1.1879 | B.61 | C.9 |
| 1.1880 | B.62 | C.9 |
| 1.1881 | B.63 | C.9 |
| 1.1882 | B.64 | C.9 |
| 1.1883 | B.65 | C.9 |
| 1.1884 | B.66 | C.9 |
| 1.1885 | B.67 | C.9 |
| 1.1886 | B.68 | C.9 |
| 1.1887 | B.69 | C.9 |
| 1.1888 | B.70 | C.9 |
| 1.1889 | B.71 | C.9 |
| 1.1890 | B.72 | C.9 |
| 1.1891 | B.73 | C.9 |
| 1.1892 | B.74 | C.9 |
| 1.1893 | B.75 | C.9 |
| 1.1894 | B.76 | C.9 |
| 1.1895 | B.77 | C.9 |
| 1.1896 | B.78 | C.9 |
| 1.1897 | B.79 | C.9 |
| 1.1898 | B.80 | C.9 |
| 1.1899 | B.81 | C.9 |
| 1.1900 | B.82 | C.9 |
| 1.1901 | B.83 | C.9 |
| 1.1902 | B.84 | C.9 |
| 1.1903 | B.85 | C.9 |
| 1.1904 | B.86 | C.9 |
| 1.1905 | B.87 | C.9 |
| 1.1906 | B.88 | C.9 |
| 1.1907 | B.89 | C.9 |
| 1.1908 | B.90 | C.9 |
| 1.1909 | B.91 | C.9 |
| 1.1910 | B.92 | C.9 |
| 1.1911 | B.93 | C.9 |
| 1.1912 | B.94 | C.9 |
| 1.1913 | B.95 | C.9 |
| 1.1914 | B.96 | C.9 |
| 1.1915 | B.97 | C.9 |
| 1.1916 | B.98 | C.9 |
| 1.1917 | B.99 | C.9 |
| 1.1918 | B.100 | C.9 |
| 1.1919 | B.101 | C.9 |
| 1.1920 | B.102 | C.9 |
| 1.1921 | B.103 | C.9 |
| 1.1922 | B.104 | C.9 |
| 1.1923 | B.105 | C.9 |
| 1.1924 | B.106 | C.9 |
| 1.1925 | B.107 | C.9 |
| 1.1926 | B.108 | C.9 |
| 1.1927 | B.109 | C.9 |
| 1.1928 | B.110 | C.9 |
| 1.1929 | B.111 | C.9 |
| 1.1930 | B.112 | C.9 |
| 1.1931 | B.113 | C.9 |
| 1.1932 | B.114 | C.9 |
| 1.1933 | B.115 | C.9 |
| 1.1934 | B.116 | C.9 |
| 1.1935 | B.117 | C.9 |
| 1.1936 | B.118 | C.9 |
| 1.1937 | B.119 | C.9 |
| 1.1938 | B.120 | C.9 |
| 1.1939 | B.121 | C.9 |
| 1.1940 | B.122 | C.9 |
| 1.1941 | B.123 | C.9 |
| 1.1942 | B.124 | C.9 |
| 1.1943 | B.125 | C.9 |
| 1.1944 | B.126 | C.9 |
| 1.1945 | B.127 | C.9 |
| 1.1946 | B.128 | C.9 |
| 1.1947 | B.129 | C.9 |
| 1.1948 | B.130 | C.9 |
| 1.1949 | B.131 | C.9 |
| 1.1950 | B.132 | C.9 |
| 1.1951 | B.133 | C.9 |
| 1.1952 | B.134 | C.9 |
| 1.1953 | B.135 | C.9 |
| 1.1954 | B.136 | C.9 |
| 1.1955 | B.137 | C.9 |
| 1.1956 | B.138 | C.9 |
| 1.1957 | B.139 | C.9 |
| 1.1958 | B.140 | C.9 |
| 1.1959 | B.141 | C.9 |
| 1.1960 | B.142 | C.9 |
| 1.1961 | B.143 | C.9 |
| 1.1962 | B.144 | C.9 |
| 1.1963 | B.145 | C.9 |
| 1.1964 | B.146 | C.9 |
| 1.1965 | B.147 | C.9 |
| 1.1966 | B.148 | C.9 |
| 1.1967 | B.149 | C.9 |
| 1.1968 | B.150 | C.9 |
| 1.1969 | B.151 | C.9 |
| 1.1970 | B.152 | C.9 |
| 1.1971 | B.153 | C.9 |
| 1.1972 | B.154 | C.9 |
| 1.1973 | B.155 | C.9 |
| 1.1974 | B.156 | C.9 |
| 1.1975 | B.157 | C.9 |
| 1.1976 | B.158 | C.9 |
| 1.1977 | B.159 | C.9 |
| 1.1978 | B.160 | C.9 |
| 1.1979 | B.161 | C.9 |
| 1.1980 | B.162 | C.9 |
| 1.1981 | B.163 | C.9 |
| 1.1982 | B.164 | C.9 |
| 1.1983 | B.165 | C.9 |
| 1.1984 | B.166 | C.9 |
| 1.1985 | B.167 | C.9 |
| 1.1986 | B.168 | C.9 |
| 1.1987 | B.169 | C.9 |
| 1.1988 | B.170 | C.9 |
| 1.1989 | B.171 | C.9 |
| 1.1990 | B.172 | C.9 |
| 1.1991 | B.173 | C.9 |
| 1.1992 | B.174 | C.9 |
| 1.1993 | B.175 | C.9 |
| 1.1994 | B.176 | C.9 |
| 1.1995 | B.177 | C.9 |
| 1.1996 | B.178 | C.9 |
| 1.1997 | B.179 | C.9 |
| 1.1998 | B.180 | C.9 |
| 1.1999 | B.181 | C.9 |
| 1.2000 | B.182 | C.9 |
| 1.2001 | B.183 | C.9 |
| 1.2002 | B.184 | C.9 |
| 1.2003 | B.185 | C.9 |
| 1.2004 | B.186 | C.9 |
| 1.2005 | B.187 | C.9 |
| 1.2006 | B.188 | C.9 |
| 1.2007 | B.189 | C.9 |
| 1.2008 | B.190 | C.9 |
| 1.2009 | B.191 | C.9 |
| 1.2010 | B.192 | C.9 |
| 1.2011 | B.193 | C.9 |
| 1.2012 | B.194 | C.9 |
| 1.2013 | B.195 | C.9 |
| 1.2014 | B.196 | C.9 |
| 1.2015 | B.197 | C.9 |
| 1.2016 | B.198 | C.9 |
| 1.2017 | B.199 | C.9 |
| 1.2018 | B.200 | C.9 |
| 1.2019 | B.201 | C.9 |
| 1.2020 | B.202 | C.9 |
| 1.2021 | B.1 | C.10 |
| 1.2022 | B.2 | C.10 |
| 1.2023 | B.3 | C.10 |
| 1.2024 | B.4 | C.10 |
| 1.2025 | B.5 | C.10 |
| 1.2026 | B.6 | C.10 |
| 1.2027 | B.7 | C.10 |
| 1.2028 | B.8 | C.10 |
| 1.2029 | B.9 | C.10 |
| 1.2030 | B.10 | C.10 |
| 1.2031 | B.11 | C.10 |
| 1.2032 | B.12 | C.10 |
| 1.2033 | B.13 | C.10 |
| 1.2034 | B.14 | C.10 |
| 1.2035 | B.15 | C.10 |
| 1.2036 | B.16 | C.10 |
| 1.2037 | B.17 | C.10 |
| 1.2038 | B.18 | C.10 |
| 1.2039 | B.19 | C.10 |
| 1.2040 | B.20 | C.10 |
| 1.2041 | B.21 | C.10 |
| 1.2042 | B.22 | C.10 |
| 1.2043 | B.23 | C.10 |
| 1.2044 | B.24 | C.10 |
| 1.2045 | B.25 | C.10 |
| 1.2046 | B.26 | C.10 |
| 1.2047 | B.27 | C.10 |
| 1.2048 | B.28 | C.10 |
| 1.2049 | B.29 | C.10 |
| 1.2050 | B.30 | C.10 |
| 1.2051 | B.31 | C.10 |
| 1.2052 | B.32 | C.10 |
| 1.2053 | B.33 | C.10 |
| 1.2054 | B.34 | C.10 |
| 1.2055 | B.35 | C.10 |
| 1.2056 | B.36 | C.10 |
| 1.2057 | B.37 | C.10 |
| 1.2058 | B.38 | C.10 |
| 1.2059 | B.39 | C.10 |
| 1.2060 | B.40 | C.10 |
| 1.2061 | B.41 | C.10 |
| 1.2062 | B.42 | C.10 |
| 1.2063 | B.43 | C.10 |
| 1.2064 | B.44 | C.10 |
| 1.2065 | B.45 | C.10 |
| 1.2066 | B.46 | C.10 |
| 1.2067 | B.47 | C.10 |
| 1.2068 | B.48 | C.10 |
| 1.2069 | B.49 | C.10 |
| 1.2070 | B.50 | C.10 |
| 1.2071 | B.51 | C.10 |
| 1.2072 | B.52 | C.10 |
| 1.2073 | B.53 | C.10 |
| 1.2074 | B.54 | C.10 |
| 1.2075 | B.55 | C.10 |
| 1.2076 | B.56 | C.10 |
| 1.2077 | B.57 | C.10 |
| 1.2078 | B.58 | C.10 |
| 1.2079 | B.59 | C.10 |
| 1.2080 | B.60 | C.10 |
| 1.2081 | B.61 | C.10 |
| 1.2082 | B.62 | C.10 |
| 1.2083 | B.63 | C.10 |
| 1.2084 | B.64 | C.10 |
| 1.2085 | B.65 | C.10 |
| 1.2086 | B.66 | C.10 |
| 1.2087 | B.67 | C.10 |
| 1.2088 | B.68 | C.10 |
| 1.2089 | B.69 | C.10 |
| 1.2090 | B.70 | C.10 |
| 1.2091 | B.71 | C.10 |
| 1.2092 | B.72 | C.10 |
| 1.2093 | B.73 | C.10 |
| 1.2094 | B.74 | C.10 |
| 1.2095 | B.75 | C.10 |
| 1.2096 | B.76 | C.10 |
| 1.2097 | B.77 | C.10 |
| 1.2098 | B.78 | C.10 |
| 1.2099 | B.79 | C.10 |
| 1.2100 | B.80 | C.10 |
| 1.2101 | B.81 | C.10 |
| 1.2102 | B.82 | C.10 |
| 1.2103 | B.83 | C.10 |
| 1.2104 | B.84 | C.10 |
| 1.2105 | B.85 | C.10 |
| 1.2106 | B.86 | C.10 |
| 1.2107 | B.87 | C.10 |
| 1.2108 | B.88 | C.10 |
| 1.2109 | B.89 | C.10 |
| 1.2110 | B.90 | C.10 |
| 1.2111 | B.91 | C.10 |
| 1.2112 | B.92 | C.10 |
| 1.2113 | B.93 | C.10 |
| 1.2114 | B.94 | C.10 |
| 1.2115 | B.95 | C.10 |
| 1.2116 | B.96 | C.10 |
| 1.2117 | B.97 | C.10 |
| 1.2118 | B.98 | C.10 |
| 1.2119 | B.99 | C.10 |
| 1.2120 | B.100 | C.10 |
| 1.2121 | B.101 | C.10 |
| 1.2122 | B.102 | C.10 |
| 1.2123 | B.103 | C.10 |
| 1.2124 | B.104 | C.10 |
| 1.2125 | B.105 | C.10 |
| 1.2126 | B.106 | C.10 |
| 1.2127 | B.107 | C.10 |
| 1.2128 | B.108 | C.10 |
| 1.2129 | B.109 | C.10 |
| 1.2130 | B.110 | C.10 |
| 1.2131 | B.111 | C.10 |
| 1.2132 | B.112 | C.10 |
| 1.2133 | B.113 | C.10 |
| 1.2134 | B.114 | C.10 |
| 1.2135 | B.115 | C.10 |
| 1.2136 | B.116 | C.10 |
| 1.2137 | B.117 | C.10 |
| 1.2138 | B.118 | C.10 |
| 1.2139 | B.119 | C.10 |
| 1.2140 | B.120 | C.10 |
| 1.2141 | B.121 | C.10 |
| 1.2142 | B.122 | C.10 |
| 1.2143 | B.123 | C.10 |
| 1.2144 | B.124 | C.10 |
| 1.2145 | B.125 | C.10 |
| 1.2146 | B.126 | C.10 |
| 1.2147 | B.127 | C.10 |
| 1.2148 | B.128 | C.10 |
| 1.2149 | B.129 | C.10 |
| 1.2150 | B.130 | C.10 |
| 1.2151 | B.131 | C.10 |
| 1.2152 | B.132 | C.10 |
| 1.2153 | B.133 | C.10 |
| 1.2154 | B.134 | C.10 |
| 1.2155 | B.135 | C.10 |
| 1.2156 | B.136 | C.10 |
| 1.2157 | B.137 | C.10 |
| 1.2158 | B.138 | C.10 |
| 1.2159 | B.139 | C.10 |
| 1.2160 | B.140 | C.10 |
| 1.2161 | B.141 | C.10 |
| 1.2162 | B.142 | C.10 |
| 1.2163 | B.143 | C.10 |
| 1.2164 | B.144 | C.10 |
| 1.2165 | B.145 | C.10 |
| 1.2166 | B.146 | C.10 |
| 1.2167 | B.147 | C.10 |
| 1.2168 | B.148 | C.10 |
| 1.2169 | B.149 | C.10 |
| 1.2170 | B.150 | C.10 |
| 1.2171 | B.151 | C.10 |
| 1.2172 | B.152 | C.10 |
| 1.2173 | B.153 | C.10 |
| 1.2174 | B.154 | C.10 |
| 1.2175 | B.155 | C.10 |
| 1.2176 | B.156 | C.10 |
| 1.2177 | B.157 | C.10 |
| 1.2178 | B.158 | C.10 |
| 1.2179 | B.159 | C.10 |
| 1.2180 | B.160 | C.10 |
| 1.2181 | B.161 | C.10 |
| 1.2182 | B.162 | C.10 |
| 1.2183 | B.163 | C.10 |
| 1.2184 | B.164 | C.10 |
| 1.2185 | B.165 | C.10 |
| 1.2186 | B.166 | C.10 |
| 1.2187 | B.167 | C.10 |
| 1.2188 | B.168 | C.10 |
| 1.2189 | B.169 | C.10 |
| 1.2190 | B.170 | C.10 |
| 1.2191 | B.171 | C.10 |
| 1.2192 | B.172 | C.10 |
| 1.2193 | B.173 | C.10 |
| 1.2194 | B.174 | C.10 |
| 1.2195 | B.175 | C.10 |
| 1.2196 | B.176 | C.10 |
| 1.2197 | B.177 | C.10 |
| 1.2198 | B.178 | C.10 |
| 1.2199 | B.179 | C.10 |
| 1.2200 | B.180 | C.10 |
| 1.2201 | B.181 | C.10 |
| 1.2202 | B.182 | C.10 |
| 1.2203 | B.183 | C.10 |
| 1.2204 | B.184 | C.10 |
| 1.2205 | B.185 | C.10 |
| 1.2206 | B.186 | C.10 |
| 1.2207 | B.187 | C.10 |
| 1.2208 | B.188 | C.10 |
| 1.2209 | B.189 | C.10 |
| 1.2210 | B.190 | C.10 |
| 1.2211 | B.191 | C.10 |
| 1.2212 | B.192 | C.10 |
| 1.2213 | B.193 | C.10 |
| 1.2214 | B.194 | C.10 |
| 1.2215 | B.195 | C.10 |
| 1.2216 | B.196 | C.10 |
| 1.2217 | B.197 | C.10 |
| 1.2218 | B.198 | C.10 |
| 1.2219 | B.199 | C.10 |
| 1.2220 | B.200 | C.10 |
| 1.2221 | B.201 | C.10 |
| 1.2222 | B.202 | C.10 |
| 1.2223 | B.1 | C.11 |
| 1.2224 | B.2 | C.11 |
| 1.2225 | B.3 | C.11 |
| 1.2226 | B.4 | C.11 |
| 1.2227 | B.5 | C.11 |
| 1.2228 | B.6 | C.11 |
| 1.2229 | B.7 | C.11 |
| 1.2230 | B.8 | C.11 |
| 1.2231 | B.9 | C.11 |
| 1.2232 | B.10 | C.11 |
| 1.2233 | B.11 | C.11 |
| 1.2234 | B.12 | C.11 |
| 1.2235 | B.13 | C.11 |
| 1.2236 | B.14 | C.11 |
| 1.2237 | B.15 | C.11 |
| 1.2238 | B.16 | C.11 |
| 1.2239 | B.17 | C.11 |
| 1.2240 | B.18 | C.11 |
| 1.2241 | B.19 | C.11 |
| 1.2242 | B.20 | C.11 |
| 1.2243 | B.21 | C.11 |
| 1.2244 | B.22 | C.11 |
| 1.2245 | B.23 | C.11 |
| 1.2246 | B.24 | C.11 |
| 1.2247 | B.25 | C.11 |
| 1.2248 | B.26 | C.11 |
| 1.2249 | B.27 | C.11 |
| 1.2250 | B.28 | C.11 |
| 1.2251 | B.29 | C.11 |
| 1.2252 | B.30 | C.11 |
| 1.2253 | B.31 | C.11 |
| 1.2254 | B.32 | C.11 |
| 1.2255 | B.33 | C.11 |
| 1.2256 | B.34 | C.11 |
| 1.2257 | B.35 | C.11 |
| 1.2258 | B.36 | C.11 |
| 1.2259 | B.37 | C.11 |
| 1.2260 | B.38 | C.11 |
| 1.2261 | B.39 | C.11 |
| 1.2262 | B.40 | C.11 |
| 1.2263 | B.41 | C.11 |
| 1.2264 | B.42 | C.11 |
| 1.2265 | B.43 | C.11 |
| 1.2266 | B.44 | C.11 |
| 1.2267 | B.45 | C.11 |
| 1.2268 | B.46 | C.11 |
| 1.2269 | B.47 | C.11 |
| 1.2270 | B.48 | C.11 |
| 1.2271 | B.49 | C.11 |
| 1.2272 | B.50 | C.11 |
| 1.2273 | B.51 | C.11 |
| 1.2274 | B.52 | C.11 |
| 1.2275 | B.53 | C.11 |
| 1.2276 | B.54 | C.11 |
| 1.2277 | B.55 | C.11 |
| 1.2278 | B.56 | C.11 |
| 1.2279 | B.57 | C.11 |
| 1.2280 | B.58 | C.11 |
| 1.2281 | B.59 | C.11 |
| 1.2282 | B.60 | C.11 |
| 1.2283 | B.61 | C.11 |
| 1.2284 | B.62 | C.11 |
| 1.2285 | B.63 | C.11 |
| 1.2286 | B.64 | C.11 |
| 1.2287 | B.65 | C.11 |
| 1.2288 | B.66 | C.11 |
| 1.2289 | B.67 | C.11 |
| 1.2290 | B.68 | C.11 |
| 1.2291 | B.69 | C.11 |
| 1.2292 | B.70 | C.11 |
| 1.2293 | B.71 | C.11 |
| 1.2294 | B.72 | C.11 |
| 1.2295 | B.73 | C.11 |
| 1.2296 | B.74 | C.11 |
| 1.2297 | B.75 | C.11 |
| 1.2298 | B.76 | C.11 |
| 1.2299 | B.77 | C.11 |
| 1.2300 | B.78 | C.11 |
| 1.2301 | B.79 | C.11 |
| 1.2302 | B.80 | C.11 |
| 1.2303 | B.81 | C.11 |
| 1.2304 | B.82 | C.11 |
| 1.2305 | B.83 | C.11 |
| 1.2306 | B.84 | C.11 |
| 1.2307 | B.85 | C.11 |
| 1.2308 | B.86 | C.11 |
| 1.2309 | B.87 | C.11 |
| 1.2310 | B.88 | C.11 |
| 1.2311 | B.89 | C.11 |
| 1.2312 | B.90 | C.11 |
| 1.2313 | B.91 | C.11 |
| 1.2314 | B.92 | C.11 |
| 1.2315 | B.93 | C.11 |
| 1.2316 | B.94 | C.11 |
| 1.2317 | B.95 | C.11 |
| 1.2318 | B.96 | C.11 |
| 1.2319 | B.97 | C.11 |
| 1.2320 | B.98 | C.11 |
| 1.2321 | B.99 | C.11 |
| 1.2322 | B.100 | C.11 |
| 1.2323 | B.101 | C.11 |
| 1.2324 | B.102 | C.11 |
| 1.2325 | B.103 | C.11 |
| 1.2326 | B.104 | C.11 |
| 1.2327 | B.105 | C.11 |
| 1.2328 | B.106 | C.11 |
| 1.2329 | B.107 | C.11 |
| 1.2330 | B.108 | C.11 |
| 1.2331 | B.109 | C.11 |
| 1.2332 | B.110 | C.11 |
| 1.2333 | B.111 | C.11 |
| 1.2334 | B.112 | C.11 |
| 1.2335 | B.113 | C.11 |
| 1.2336 | B.114 | C.11 |
| 1.2337 | B.115 | C.11 |
| 1.2338 | B.116 | C.11 |
| 1.2339 | B.117 | C.11 |
| 1.2340 | B.118 | C.11 |
| 1.2341 | B.119 | C.11 |
| 1.2342 | B.120 | C.11 |
| 1.2343 | B.121 | C.11 |
| 1.2344 | B.122 | C.11 |
| 1.2345 | B.123 | C.11 |
| 1.2346 | B.124 | C.11 |
| 1.2347 | B.125 | C.11 |
| 1.2348 | B.126 | C.11 |
| 1.2349 | B.127 | C.11 |
| 1.2350 | B.128 | C.11 |
| 1.2351 | B.129 | C.11 |
| 1.2352 | B.130 | C.11 |
| 1.2353 | B.131 | C.11 |
| 1.2354 | B.132 | C.11 |
| 1.2355 | B.133 | C.11 |
| 1.2356 | B.134 | C.11 |
| 1.2357 | B.135 | C.11 |
| 1.2358 | B.136 | C.11 |
| 1.2359 | B.137 | C.11 |
| 1.2360 | B.138 | C.11 |
| 1.2361 | B.139 | C.11 |
| 1.2362 | B.140 | C.11 |
| 1.2363 | B.141 | C.11 |
| 1.2364 | B.142 | C.11 |
| 1.2365 | B.143 | C.11 |
| 1.2366 | B.144 | C.11 |
| 1.2367 | B.145 | C.11 |
| 1.2368 | B.146 | C.11 |
| 1.2369 | B.147 | C.11 |
| 1.2370 | B.148 | C.11 |
| 1.2371 | B.149 | C.11 |
| 1.2372 | B.150 | C.11 |
| 1.2373 | B.151 | C.11 |
| 1.2374 | B.152 | C.11 |
| 1.2375 | B.153 | C.11 |
| 1.2376 | B.154 | C.11 |
| 1.2377 | B.155 | C.11 |
| 1.2378 | B.156 | C.11 |
| 1.2379 | B.157 | C.11 |
| 1.2380 | B.158 | C.11 |
| 1.2381 | B.159 | C.11 |
| 1.2382 | B.160 | C.11 |
| 1.2383 | B.161 | C.11 |
| 1.2384 | B.162 | C.11 |
| 1.2385 | B.163 | C.11 |
| 1.2386 | B.164 | C.11 |
| 1.2387 | B.165 | C.11 |
| 1.2388 | B.166 | C.11 |
| 1.2389 | B.167 | C.11 |
| 1.2390 | B.168 | C.11 |
| 1.2391 | B.169 | C.11 |
| 1.2392 | B.170 | C.11 |
| 1.2393 | B.171 | C.11 |
| 1.2394 | B.172 | C.11 |
| 1.2395 | B.173 | C.11 |
| 1.2396 | B.174 | C.11 |
| 1.2397 | B.175 | C.11 |
| 1.2398 | B.176 | C.11 |
| 1.2399 | B.177 | C.11 |
| 1.2400 | B.178 | C.11 |
| 1.2401 | B.179 | C.11 |
| 1.2402 | B.180 | C.11 |
| 1.2403 | B.181 | C.11 |
| 1.2404 | B.182 | C.11 |
| 1.2405 | B.183 | C.11 |
| 1.2406 | B.184 | C.11 |
| 1.2407 | B.185 | C.11 |
| 1.2408 | B.186 | C.11 |
| 1.2409 | B.187 | C.11 |
| 1.2410 | B.188 | C.11 |
| 1.2411 | B.189 | C.11 |
| 1.2412 | B.190 | C.11 |
| 1.2413 | B.191 | C.11 |
| 1.2414 | B.192 | C.11 |
| 1.2415 | B.193 | C.11 |
| 1.2416 | B.194 | C.11 |
| 1.2417 | B.195 | C.11 |
| 1.2418 | B.196 | C.11 |
| 1.2419 | B.197 | C.11 |
| 1.2420 | B.198 | C.11 |
| 1.2421 | B.199 | C.11 |
| 1.2422 | B.200 | C.11 |
| 1.2423 | B.201 | C.11 |
| 1.2424 | B.202 | C.11 |
| 1.2425 | B.1 | C.12 |
| 1.2426 | B.2 | C.12 |
| 1.2427 | B.3 | C.12 |
| 1.2428 | B.4 | C.12 |
| 1.2429 | B.5 | C.12 |
| 1.2430 | B.6 | C.12 |
| 1.2431 | B.7 | C.12 |
| 1.2432 | B.8 | C.12 |
| 1.2433 | B.9 | C.12 |
| 1.2434 | B.10 | C.12 |
| 1.2435 | B.11 | C.12 |
| 1.2436 | B.12 | C.12 |
| 1.2437 | B.13 | C.12 |
| 1.2438 | B.14 | C.12 |
| 1.2439 | B.15 | C.12 |
| 1.2440 | B.16 | C.12 |
| 1.2441 | B.17 | C.12 |
| 1.2442 | B.18 | C.12 |
| 1.2443 | B.19 | C.12 |
| 1.2444 | B.20 | C.12 |
| 1.2445 | B.21 | C.12 |
| 1.2446 | B.22 | C.12 |
| 1.2447 | B.23 | C.12 |
| 1.2448 | B.24 | C.12 |
| 1.2449 | B.25 | C.12 |
| 1.2450 | B.26 | C.12 |
| 1.2451 | B.27 | C.12 |
| 1.2452 | B.28 | C.12 |
| 1.2453 | B.29 | C.12 |
| 1.2454 | B.30 | C.12 |
| 1.2455 | B.31 | C.12 |
| 1.2456 | B.32 | C.12 |
| 1.2457 | B.33 | C.12 |
| 1.2458 | B.34 | C.12 |
| 1.2459 | B.35 | C.12 |
| 1.2460 | B.36 | C.12 |
| 1.2461 | B.37 | C.12 |
| 1.2462 | B.38 | C.12 |
| 1.2463 | B.39 | C.12 |
| 1.2464 | B.40 | C.12 |
| 1.2465 | B.41 | C.12 |
| 1.2466 | B.42 | C.12 |
| 1.2467 | B.43 | C.12 |
| 1.2468 | B.44 | C.12 |
| 1.2469 | B.45 | C.12 |
| 1.2470 | B.46 | C.12 |
| 1.2471 | B.47 | C.12 |
| 1.2472 | B.48 | C.12 |
| 1.2473 | B.49 | C.12 |
| 1.2474 | B.50 | C.12 |
| 1.2475 | B.51 | C.12 |
| 1.2476 | B.52 | C.12 |
| 1.2477 | B.53 | C.12 |
| 1.2478 | B.54 | C.12 |
| 1.2479 | B.55 | C.12 |
| 1.2480 | B.56 | C.12 |
| 1.2481 | B.57 | C.12 |
| 1.2482 | B.58 | C.12 |
| 1.2483 | B.59 | C.12 |
| 1.2484 | B.60 | C.12 |
| 1.2485 | B.61 | C.12 |
| 1.2486 | B.62 | C.12 |
| 1.2487 | B.63 | C.12 |
| 1.2488 | B.64 | C.12 |
| 1.2489 | B.65 | C.12 |
| 1.2490 | B.66 | C.12 |
| 1.2491 | B.67 | C.12 |
| 1.2492 | B.68 | C.12 |
| 1.2493 | B.69 | C.12 |
| 1.2494 | B.70 | C.12 |
| 1.2495 | B.71 | C.12 |
| 1.2496 | B.72 | C.12 |
| 1.2497 | B.73 | C.12 |
| 1.2498 | B.74 | C.12 |
| 1.2499 | B.75 | C.12 |
| 1.2500 | B.76 | C.12 |
| 1.2501 | B.77 | C.12 |
| 1.2502 | B.78 | C.12 |
| 1.2503 | B.79 | C.12 |
| 1.2504 | B.80 | C.12 |
| 1.2505 | B.81 | C.12 |
| 1.2506 | B.82 | C.12 |
| 1.2507 | B.83 | C.12 |
| 1.2508 | B.84 | C.12 |
| 1.2509 | B.85 | C.12 |
| 1.2510 | B.86 | C.12 |
| 1.2511 | B.87 | C.12 |
| 1.2512 | B.88 | C.12 |
| 1.2513 | B.89 | C.12 |
| 1.2514 | B.90 | C.12 |
| 1.2515 | B.91 | C.12 |
| 1.2516 | B.92 | C.12 |
| 1.2517 | B.93 | C.12 |
| 1.2518 | B.94 | C.12 |
| 1.2519 | B.95 | C.12 |
| 1.2520 | B.96 | C.12 |
| 1.2521 | B.97 | C.12 |
| 1.2522 | B.98 | C.12 |
| 1.2523 | B.99 | C.12 |
| 1.2524 | B.100 | C.12 |
| 1.2525 | B.101 | C.12 |
| 1.2526 | B.102 | C.12 |
| 1.2527 | B.103 | C.12 |
| 1.2528 | B.104 | C.12 |
| 1.2529 | B.105 | C.12 |
| 1.2530 | B.106 | C.12 |
| 1.2531 | B.107 | C.12 |
| 1.2532 | B.108 | C.12 |
| 1.2533 | B.109 | C.12 |
| 1.2534 | B.110 | C.12 |
| 1.2535 | B.111 | C.12 |
| 1.2536 | B.112 | C.12 |
| 1.2537 | B.113 | C.12 |
| 1.2538 | B.114 | C.12 |
| 1.2539 | B.115 | C.12 |
| 1.2540 | B.116 | C.12 |
| 1.2541 | B.117 | C.12 |
| 1.2542 | B.118 | C.12 |
| 1.2543 | B.119 | C.12 |
| 1.2544 | B.120 | C.12 |
| 1.2545 | B.121 | C.12 |
| 1.2546 | B.122 | C.12 |
| 1.2547 | B.123 | C.12 |
| 1.2548 | B.124 | C.12 |
| 1.2549 | B.125 | C.12 |
| 1.2550 | B.126 | C.12 |
| 1.2551 | B.127 | C.12 |
| 1.2552 | B.128 | C.12 |
| 1.2553 | B.129 | C.12 |
| 1.2554 | B.130 | C.12 |
| 1.2555 | B.131 | C.12 |
| 1.2556 | B.132 | C.12 |
| 1.2557 | B.133 | C.12 |
| 1.2558 | B.134 | C.12 |
| 1.2559 | B.135 | C.12 |
| 1.2560 | B.136 | C.12 |
| 1.2561 | B.137 | C.12 |
| 1.2562 | B.138 | C.12 |
| 1.2563 | B.139 | C.12 |
| 1.2564 | B.140 | C.12 |
| 1.2565 | B.141 | C.12 |
| 1.2566 | B.142 | C.12 |
| 1.2567 | B.143 | C.12 |
| 1.2568 | B.144 | C.12 |
| 1.2569 | B.145 | C.12 |
| 1.2570 | B.146 | C.12 |
| 1.2571 | B.147 | C.12 |
| 1.2572 | B.148 | C.12 |
| 1.2573 | B.149 | C.12 |
| 1.2574 | B.150 | C.12 |
| 1.2575 | B.151 | C.12 |
| 1.2576 | B.152 | C.12 |
| 1.2577 | B.153 | C.12 |
| 1.2578 | B.154 | C.12 |
| 1.2579 | B.155 | C.12 |
| 1.2580 | B.156 | C.12 |
| 1.2581 | B.157 | C.12 |
| 1.2582 | B.158 | C.12 |
| 1.2583 | B.159 | C.12 |
| 1.2584 | B.160 | C.12 |
| 1.2585 | B.161 | C.12 |
| 1.2586 | B.162 | C.12 |
| 1.2587 | B.163 | C.12 |
| 1.2588 | B.164 | C.12 |
| 1.2589 | B.165 | C.12 |
| 1.2590 | B.166 | C.12 |
| 1.2591 | B.167 | C.12 |
| 1.2592 | B.168 | C.12 |
| 1.2593 | B.169 | C.12 |
| 1.2594 | B.170 | C.12 |
| 1.2595 | B.171 | C.12 |
| 1.2596 | B.172 | C.12 |
| 1.2597 | B.173 | C.12 |
| 1.2598 | B.174 | C.12 |
| 1.2599 | B.175 | C.12 |
| 1.2600 | B.176 | C.12 |
| 1.2601 | B.177 | C.12 |
| 1.2602 | B.178 | C.12 |
| 1.2603 | B.179 | C.12 |
| 1.2604 | B.180 | C.12 |
| 1.2605 | B.181 | C.12 |
| 1.2606 | B.182 | C.12 |
| 1.2607 | B.183 | C.12 |
| 1.2608 | B.184 | C.12 |
| 1.2609 | B.185 | C.12 |
| 1.2610 | B.186 | C.12 |
| 1.2611 | B.187 | C.12 |
| 1.2612 | B.188 | C.12 |
| 1.2613 | B.189 | C.12 |
| 1.2614 | B.190 | C.12 |
| 1.2615 | B.191 | C.12 |
| 1.2616 | B.192 | C.12 |
| 1.2617 | B.193 | C.12 |
| 1.2618 | B.194 | C.12 |
| 1.2619 | B.195 | C.12 |
| 1.2620 | B.196 | C.12 |
| 1.2621 | B.197 | C.12 |
| 1.2622 | B.198 | C.12 |
| 1.2623 | B.199 | C.12 |
| 1.2624 | B.200 | C.12 |
| 1.2625 | B.201 | C.12 |
| 1.2626 | B.202 | C.12 |
| 1.2627 | B.1 | C.13 |
| 1.2628 | B.2 | C.13 |
| 1.2629 | B.3 | C.13 |
| 1.2630 | B.4 | C.13 |
| 1.2631 | B.5 | C.13 |
| 1.2632 | B.6 | C.13 |
| 1.2633 | B.7 | C.13 |
| 1.2634 | B.8 | C.13 |
| 1.2635 | B.9 | C.13 |
| 1.2636 | B.10 | C.13 |
| 1.2637 | B.11 | C.13 |
| 1.2638 | B.12 | C.13 |
| 1.2639 | B.13 | C.13 |
| 1.2640 | B.14 | C.13 |
| 1.2641 | B.15 | C.13 |
| 1.2642 | B.16 | C.13 |
| 1.2643 | B.17 | C.13 |
| 1.2644 | B.18 | C.13 |
| 1.2645 | B.19 | C.13 |
| 1.2646 | B.20 | C.13 |
| 1.2647 | B.21 | C.13 |
| 1.2648 | B.22 | C.13 |
| 1.2649 | B.23 | C.13 |
| 1.2650 | B.24 | C.13 |
| 1.2651 | B.25 | C.13 |
| 1.2652 | B.26 | C.13 |
| 1.2653 | B.27 | C.13 |
| 1.2654 | B.28 | C.13 |
| 1.2655 | B.29 | C.13 |
| 1.2656 | B.30 | C.13 |
| 1.2657 | B.31 | C.13 |
| 1.2658 | B.32 | C.13 |
| 1.2659 | B.33 | C.13 |
| 1.2660 | B.34 | C.13 |
| 1.2661 | B.35 | C.13 |
| 1.2662 | B.36 | C.13 |
| 1.2663 | B.37 | C.13 |
| 1.2664 | B.38 | C.13 |
| 1.2665 | B.39 | C.13 |
| 1.2666 | B.40 | C.13 |
| 1.2667 | B.41 | C.13 |
| 1.2668 | B.42 | C.13 |
| 1.2669 | B.43 | C.13 |
| 1.2670 | B.44 | C.13 |
| 1.2671 | B.45 | C.13 |
| 1.2672 | B.46 | C.13 |
| 1.2673 | B.47 | C.13 |
| 1.2674 | B.48 | C.13 |
| 1.2675 | B.49 | C.13 |
| 1.2676 | B.50 | C.13 |
| 1.2677 | B.51 | C.13 |
| 1.2678 | B.52 | C.13 |
| 1.2679 | B.53 | C.13 |
| 1.2680 | B.54 | C.13 |
| 1.2681 | B.55 | C.13 |
| 1.2682 | B.56 | C.13 |
| 1.2683 | B.57 | C.13 |
| 1.2684 | B.58 | C.13 |
| 1.2685 | B.59 | C.13 |
| 1.2686 | B.60 | C.13 |
| 1.2687 | B.61 | C.13 |
| 1.2688 | B.62 | C.13 |
| 1.2689 | B.63 | C.13 |
| 1.2690 | B.64 | C.13 |
| 1.2691 | B.65 | C.13 |
| 1.2692 | B.66 | C.13 |
| 1.2693 | B.67 | C.13 |
| 1.2694 | B.68 | C.13 |
| 1.2695 | B.69 | C.13 |
| 1.2696 | B.70 | C.13 |
| 1.2697 | B.71 | C.13 |
| 1.2698 | B.72 | C.13 |
| 1.2699 | B.73 | C.13 |
| 1.2700 | B.74 | C.13 |
| 1.2701 | B.75 | C.13 |
| 1.2702 | B.76 | C.13 |
| 1.2703 | B.77 | C.13 |
| 1.2704 | B.78 | C.13 |
| 1.2705 | B.79 | C.13 |
| 1.2706 | B.80 | C.13 |
| 1.2707 | B.81 | C.13 |
| 1.2708 | B.82 | C.13 |
| 1.2709 | B.83 | C.13 |
| 1.2710 | B.84 | C.13 |
| 1.2711 | B.85 | C.13 |
| 1.2712 | B.86 | C.13 |
| 1.2713 | B.87 | C.13 |
| 1.2714 | B.88 | C.13 |
| 1.2715 | B.89 | C.13 |
| 1.2716 | B.90 | C.13 |
| 1.2717 | B.91 | C.13 |
| 1.2718 | B.92 | C.13 |
| 1.2719 | B.93 | C.13 |
| 1.2720 | B.94 | C.13 |
| 1.2721 | B.95 | C.13 |
| 1.2722 | B.96 | C.13 |
| 1.2723 | B.97 | C.13 |
| 1.2724 | B.98 | C.13 |
| 1.2725 | B.99 | C.13 |
| 1.2726 | B.100 | C.13 |
| 1.2727 | B.101 | C.13 |
| 1.2728 | B.102 | C.13 |
| 1.2729 | B.103 | C.13 |
| 1.2730 | B.104 | C.13 |
| 1.2731 | B.105 | C.13 |
| 1.2732 | B.106 | C.13 |
| 1.2733 | B.107 | C.13 |
| 1.2734 | B.108 | C.13 |
| 1.2735 | B.109 | C.13 |
| 1.2736 | B.110 | C.13 |
| 1.2737 | B.111 | C.13 |
| 1.2738 | B.112 | C.13 |
| 1.2739 | B.113 | C.13 |
| 1.2740 | B.114 | C.13 |
| 1.2741 | B.115 | C.13 |
| 1.2742 | B.116 | C.13 |
| 1.2743 | B.117 | C.13 |
| 1.2744 | B.118 | C.13 |
| 1.2745 | B.119 | C.13 |
| 1.2746 | B.120 | C.13 |
| 1.2747 | B.121 | C.13 |
| 1.2748 | B.122 | C.13 |
| 1.2749 | B.123 | C.13 |
| 1.2750 | B.124 | C.13 |
| 1.2751 | B.125 | C.13 |
| 1.2752 | B.126 | C.13 |
| 1.2753 | B.127 | C.13 |
| 1.2754 | B.128 | C.13 |
| 1.2755 | B.129 | C.13 |
| 1.2756 | B.130 | C.13 |
| 1.2757 | B.131 | C.13 |
| 1.2758 | B.132 | C.13 |
| 1.2759 | B.133 | C.13 |
| 1.2760 | B.134 | C.13 |
| 1.2761 | B.135 | C.13 |
| 1.2762 | B.136 | C.13 |
| 1.2763 | B.137 | C.13 |
| 1.2764 | B.138 | C.13 |
| 1.2765 | B.139 | C.13 |
| 1.2766 | B.140 | C.13 |
| 1.2767 | B.141 | C.13 |
| 1.2768 | B.142 | C.13 |
| 1.2769 | B.143 | C.13 |
| 1.2770 | B.144 | C.13 |
| 1.2771 | B.145 | C.13 |
| 1.2772 | B.146 | C.13 |
| 1.2773 | B.147 | C.13 |
| 1.2774 | B.148 | C.13 |
| 1.2775 | B.149 | C.13 |
| 1.2776 | B.150 | C.13 |
| 1.2777 | B.151 | C.13 |
| 1.2778 | B.152 | C.13 |
| 1.2779 | B.153 | C.13 |
| 1.2780 | B.154 | C.13 |
| 1.2781 | B.155 | C.13 |
| 1.2782 | B.156 | C.13 |
| 1.2783 | B.157 | C.13 |
| 1.2784 | B.158 | C.13 |
| 1.2785 | B.159 | C.13 |
| 1.2786 | B.160 | C.13 |
| 1.2787 | B.161 | C.13 |
| 1.2788 | B.162 | C.13 |
| 1.2789 | B.163 | C.13 |
| 1.2790 | B.164 | C.13 |
| 1.2791 | B.165 | C.13 |
| 1.2792 | B.166 | C.13 |
| 1.2793 | B.167 | C.13 |
| 1.2794 | B.168 | C.13 |
| 1.2795 | B.169 | C.13 |
| 1.2796 | B.170 | C.13 |
| 1.2797 | B.171 | C.13 |
| 1.2798 | B.172 | C.13 |
| 1.2799 | B.173 | C.13 |
| 1.2800 | B.174 | C.13 |
| 1.2801 | B.175 | C.13 |
| 1.2802 | B.176 | C.13 |
| 1.2803 | B.177 | C.13 |
| 1.2804 | B.178 | C.13 |
| 1.2805 | B.179 | C.13 |
| 1.2806 | B.180 | C.13 |
| 1.2807 | B.181 | C.13 |
| 1.2808 | B.182 | C.13 |
| 1.2809 | B.183 | C.13 |
| 1.2810 | B.184 | C.13 |
| 1.2811 | B.185 | C.13 |
| 1.2812 | B.186 | C.13 |
| 1.2813 | B.187 | C.13 |
| 1.2814 | B.188 | C.13 |
| 1.2815 | B.189 | C.13 |
| 1.2816 | B.190 | C.13 |
| 1.2817 | B.191 | C.13 |
| 1.2818 | B.192 | C.13 |
| 1.2819 | B.193 | C.13 |
| 1.2820 | B.194 | C.13 |
| 1.2821 | B.195 | C.13 |
| 1.2822 | B.196 | C.13 |
| 1.2823 | B.197 | C.13 |
| 1.2824 | B.198 | C.13 |
| 1.2825 | B.199 | C.13 |
| 1.2826 | B.200 | C.13 |
| 1.2827 | B.201 | C.13 |
| 1.2828 | B.202 | C.13 |
| 1.2829 | B.1 | C.14 |
| 1.2830 | B.2 | C.14 |
| 1.2831 | B.3 | C.14 |
| 1.2832 | B.4 | C.14 |
| 1.2833 | B.5 | C.14 |
| 1.2834 | B.6 | C.14 |
| 1.2835 | B.7 | C.14 |
| 1.2836 | B.8 | C.14 |
| 1.2837 | B.9 | C.14 |
| 1.2838 | B.10 | C.14 |
| 1.2839 | B.11 | C.14 |
| 1.2840 | B.12 | C.14 |
| 1.2841 | B.13 | C.14 |
| 1.2842 | B.14 | C.14 |
| 1.2843 | B.15 | C.14 |
| 1.2844 | B.16 | C.14 |
| 1.2845 | B.17 | C.14 |
| 1.2846 | B.18 | C.14 |
| 1.2847 | B.19 | C.14 |
| 1.2848 | B.20 | C.14 |
| 1.2849 | B.21 | C.14 |
| 1.2850 | B.22 | C.14 |
| 1.2851 | B.23 | C.14 |
| 1.2852 | B.24 | C.14 |
| 1.2853 | B.25 | C.14 |
| 1.2854 | B.26 | C.14 |
| 1.2855 | B.27 | C.14 |
| 1.2856 | B.28 | C.14 |
| 1.2857 | B.29 | C.14 |
| 1.2858 | B.30 | C.14 |
| 1.2859 | B.31 | C.14 |
| 1.2860 | B.32 | C.14 |
| 1.2861 | B.33 | C.14 |
| 1.2862 | B.34 | C.14 |
| 1.2863 | B.35 | C.14 |
| 1.2864 | B.36 | C.14 |
| 1.2865 | B.37 | C.14 |
| 1.2866 | B.38 | C.14 |
| 1.2867 | B.39 | C.14 |
| 1.2868 | B.40 | C.14 |
| 1.2869 | B.41 | C.14 |
| 1.2870 | B.42 | C.14 |
| 1.2871 | B.43 | C.14 |
| 1.2872 | B.44 | C.14 |
| 1.2873 | B.45 | C.14 |
| 1.2874 | B.46 | C.14 |
| 1.2875 | B.47 | C.14 |
| 1.2876 | B.48 | C.14 |
| 1.2877 | B.49 | C.14 |
| 1.2878 | B.50 | C.14 |
| 1.2879 | B.51 | C.14 |
| 1.2880 | B.52 | C.14 |
| 1.2881 | B.53 | C.14 |
| 1.2882 | B.54 | C.14 |
| 1.2883 | B.55 | C.14 |
| 1.2884 | B.56 | C.14 |
| 1.2885 | B.57 | C.14 |
| 1.2886 | B.58 | C.14 |
| 1.2887 | B.59 | C.14 |
| 1.2888 | B.60 | C.14 |
| 1.2889 | B.61 | C.14 |
| 1.2890 | B.62 | C.14 |
| 1.2891 | B.63 | C.14 |
| 1.2892 | B.64 | C.14 |
| 1.2893 | B.65 | C.14 |
| 1.2894 | B.66 | C.14 |
| 1.2895 | B.67 | C.14 |
| 1.2896 | B.68 | C.14 |
| 1.2897 | B.69 | C.14 |
| 1.2898 | B.70 | C.14 |
| 1.2899 | B.71 | C.14 |
| 1.2900 | B.72 | C.14 |
| 1.2901 | B.73 | C.14 |
| 1.2902 | B.74 | C.14 |
| 1.2903 | B.75 | C.14 |
| 1.2904 | B.76 | C.14 |
| 1.2905 | B.77 | C.14 |
| 1.2906 | B.78 | C.14 |
| 1.2907 | B.79 | C.14 |
| 1.2908 | B.80 | C.14 |
| 1.2909 | B.81 | C.14 |
| 1.2910 | B.82 | C.14 |
| 1.2911 | B.83 | C.14 |
| 1.2912 | B.84 | C.14 |
| 1.2913 | B.85 | C.14 |
| 1.2914 | B.86 | C.14 |
| 1.2915 | B.87 | C.14 |
| 1.2916 | B.88 | C.14 |
| 1.2917 | B.89 | C.14 |
| 1.2918 | B.90 | C.14 |
| 1.2919 | B.91 | C.14 |
| 1.2920 | B.92 | C.14 |
| 1.2921 | B.93 | C.14 |
| 1.2922 | B.94 | C.14 |
| 1.2923 | B.95 | C.14 |
| 1.2924 | B.96 | C.14 |
| 1.2925 | B.97 | C.14 |
| 1.2926 | B.98 | C.14 |
| 1.2927 | B.99 | C.14 |
| 1.2928 | B.100 | C.14 |
| 1.2929 | B.101 | C.14 |
| 1.2930 | B.102 | C.14 |
| 1.2931 | B.103 | C.14 |
| 1.2932 | B.104 | C.14 |
| 1.2933 | B.105 | C.14 |
| 1.2934 | B.106 | C.14 |
| 1.2935 | B.107 | C.14 |
| 1.2936 | B.108 | C.14 |
| 1.2937 | B.109 | C.14 |
| 1.2938 | B.110 | C.14 |
| 1.2939 | B.111 | C.14 |
| 1.2940 | B.112 | C.14 |
| 1.2941 | B.113 | C.14 |
| 1.2942 | B.114 | C.14 |
| 1.2943 | B.115 | C.14 |
| 1.2944 | B.116 | C.14 |
| 1.2945 | B.117 | C.14 |
| 1.2946 | B.118 | C.14 |
| 1.2947 | B.119 | C.14 |
| 1.2948 | B.120 | C.14 |
| 1.2949 | B.121 | C.14 |
| 1.2950 | B.122 | C.14 |
| 1.2951 | B.123 | C.14 |
| 1.2952 | B.124 | C.14 |
| 1.2953 | B.125 | C.14 |
| 1.2954 | B.126 | C.14 |
| 1.2955 | B.127 | C.14 |
| 1.2956 | B.128 | C.14 |
| 1.2957 | B.129 | C.14 |
| 1.2958 | B.130 | C.14 |
| 1.2959 | B.131 | C.14 |
| 1.2960 | B.132 | C.14 |
| 1.2961 | B.133 | C.14 |
| 1.2962 | B.134 | C.14 |
| 1.2963 | B.135 | C.14 |
| 1.2964 | B.136 | C.14 |
| 1.2965 | B.137 | C.14 |
| 1.2966 | B.138 | C.14 |
| 1.2967 | B.139 | C.14 |
| 1.2968 | B.140 | C.14 |
| 1.2969 | B.141 | C.14 |
| 1.2970 | B.142 | C.14 |
| 1.2971 | B.143 | C.14 |
| 1.2972 | B.144 | C.14 |
| 1.2973 | B.145 | C.14 |
| 1.2974 | B.146 | C.14 |
| 1.2975 | B.147 | C.14 |
| 1.2976 | B.148 | C.14 |
| 1.2977 | B.149 | C.14 |
| 1.2978 | B.150 | C.14 |
| 1.2979 | B.151 | C.14 |
| 1.2980 | B.152 | C.14 |
| 1.2981 | B.153 | C.14 |
| 1.2982 | B.154 | C.14 |
| 1.2983 | B.155 | C.14 |
| 1.2984 | B.156 | C.14 |
| 1.2985 | B.157 | C.14 |
| 1.2986 | B.158 | C.14 |
| 1.2987 | B.159 | C.14 |
| 1.2988 | B.160 | C.14 |
| 1.2989 | B.161 | C.14 |
| 1.2990 | B.162 | C.14 |
| 1.2991 | B.163 | C.14 |
| 1.2992 | B.164 | C.14 |
| 1.2993 | B.165 | C.14 |
| 1.2994 | B.166 | C.14 |
| 1.2995 | B.167 | C.14 |
| 1.2996 | B.168 | C.14 |
| 1.2997 | B.169 | C.14 |
| 1.2998 | B.170 | C.14 |
| 1.2999 | B.171 | C.14 |
| 1.3000 | B.172 | C.14 |
| 1.3001 | B.173 | C.14 |
| 1.3002 | B.174 | C.14 |
| 1.3003 | B.175 | C.14 |
| 1.3004 | B.176 | C.14 |
| 1.3005 | B.177 | C.14 |
| 1.3006 | B.178 | C.14 |
| 1.3007 | B.179 | C.14 |
| 1.3008 | B.180 | C.14 |
| 1.3009 | B.181 | C.14 |
| 1.3010 | B.182 | C.14 |
| 1.3011 | B.183 | C.14 |
| 1.3012 | B.184 | C.14 |
| 1.3013 | B.185 | C.14 |
| 1.3014 | B.186 | C.14 |
| 1.3015 | B.187 | C.14 |
| 1.3016 | B.188 | C.14 |
| 1.3017 | B.189 | C.14 |
| 1.3018 | B.190 | C.14 |
| 1.3019 | B.191 | C.14 |
| 1.3020 | B.192 | C.14 |
| 1.3021 | B.193 | C.14 |
| 1.3022 | B.194 | C.14 |
| 1.3023 | B.195 | C.14 |
| 1.3024 | B.196 | C.14 |
| 1.3025 | B.197 | C.14 |
| 1.3026 | B.198 | C.14 |
| 1.3027 | B.199 | C.14 |
| 1.3028 | B.200 | C.14 |
| 1.3029 | B.201 | C.14 |
| 1.3030 | B.202 | C.14 |
| 1.3031 | B.1 | C.15 |
| 1.3032 | B.2 | C.15 |
| 1.3033 | B.3 | C.15 |
| 1.3034 | B.4 | C.15 |
| 1.3035 | B.5 | C.15 |
| 1.3036 | B.6 | C.15 |
| 1.3037 | B.7 | C.15 |
| 1.3038 | B.8 | C.15 |
| 1.3039 | B.9 | C.15 |
| 1.3040 | B.10 | C.15 |
| 1.3041 | B.11 | C.15 |
| 1.3042 | B.12 | C.15 |
| 1.3043 | B.13 | C.15 |
| 1.3044 | B.14 | C.15 |
| 1.3045 | B.15 | C.15 |
| 1.3046 | B.16 | C.15 |
| 1.3047 | B.17 | C.15 |
| 1.3048 | B.18 | C.15 |
| 1.3049 | B.19 | C.15 |
| 1.3050 | B.20 | C.15 |
| 1.3051 | B.21 | C.15 |
| 1.3052 | B.22 | C.15 |
| 1.3053 | B.23 | C.15 |
| 1.3054 | B.24 | C.15 |
| 1.3055 | B.25 | C.15 |
| 1.3056 | B.26 | C.15 |
| 1.3057 | B.27 | C.15 |
| 1.3058 | B.28 | C.15 |
| 1.3059 | B.29 | C.15 |
| 1.3060 | B.30 | C.15 |
| 1.3061 | B.31 | C.15 |
| 1.3062 | B.32 | C.15 |
| 1.3063 | B.33 | C.15 |
| 1.3064 | B.34 | C.15 |
| 1.3065 | B.35 | C.15 |
| 1.3066 | B.36 | C.15 |
| 1.3067 | B.37 | C.15 |
| 1.3068 | B.38 | C.15 |
| 1.3069 | B.39 | C.15 |
| 1.3070 | B.40 | C.15 |
| 1.3071 | B.41 | C.15 |
| 1.3072 | B.42 | C.15 |
| 1.3073 | B.43 | C.15 |
| 1.3074 | B.44 | C.15 |
| 1.3075 | B.45 | C.15 |
| 1.3076 | B.46 | C.15 |
| 1.3077 | B.47 | C.15 |
| 1.3078 | B.48 | C.15 |
| 1.3079 | B.49 | C.15 |
| 1.3080 | B.50 | C.15 |
| 1.3081 | B.51 | C.15 |
| 1.3082 | B.52 | C.15 |
| 1.3083 | B.53 | C.15 |
| 1.3084 | B.54 | C.15 |
| 1.3085 | B.55 | C.15 |
| 1.3086 | B.56 | C.15 |
| 1.3087 | B.57 | C.15 |
| 1.3088 | B.58 | C.15 |
| 1.3089 | B.59 | C.15 |
| 1.3090 | B.60 | C.15 |
| 1.3091 | B.61 | C.15 |
| 1.3092 | B.62 | C.15 |
| 1.3093 | B.63 | C.15 |
| 1.3094 | B.64 | C.15 |
| 1.3095 | B.65 | C.15 |
| 1.3096 | B.66 | C.15 |
| 1.3097 | B.67 | C.15 |
| 1.3098 | B.68 | C.15 |
| 1.3099 | B.69 | C.15 |
| 1.3100 | B.70 | C.15 |
| 1.3101 | B.71 | C.15 |
| 1.3102 | B.72 | C.15 |
| 1.3103 | B.73 | C.15 |
| 1.3104 | B.74 | C.15 |
| 1.3105 | B.75 | C.15 |
| 1.3106 | B.76 | C.15 |
| 1.3107 | B.77 | C.15 |
| 1.3108 | B.78 | C.15 |
| 1.3109 | B.79 | C.15 |
| 1.3110 | B.80 | C.15 |
| 1.3111 | B.81 | C.15 |
| 1.3112 | B.82 | C.15 |
| 1.3113 | B.83 | C.15 |
| 1.3114 | B.84 | C.15 |
| 1.3115 | B.85 | C.15 |
| 1.3116 | B.86 | C.15 |
| 1.3117 | B.87 | C.15 |
| 1.3118 | B.88 | C.15 |
| 1.3119 | B.89 | C.15 |
| 1.3120 | B.90 | C.15 |
| 1.3121 | B.91 | C.15 |
| 1.3122 | B.92 | C.15 |
| 1.3123 | B.93 | C.15 |
| 1.3124 | B.94 | C.15 |
| 1.3125 | B.95 | C.15 |
| 1.3126 | B.96 | C.15 |
| 1.3127 | B.97 | C.15 |
| 1.3128 | B.98 | C.15 |
| 1.3129 | B.99 | C.15 |
| 1.3130 | B.100 | C.15 |
| 1.3131 | B.101 | C.15 |
| 1.3132 | B.102 | C.15 |
| 1.3133 | B.103 | C.15 |
| 1.3134 | B.104 | C.15 |
| 1.3135 | B.105 | C.15 |
| 1.3136 | B.106 | C.15 |
| 1.3137 | B.107 | C.15 |
| 1.3138 | B.108 | C.15 |
| 1.3139 | B.109 | C.15 |
| 1.3140 | B.110 | C.15 |
| 1.3141 | B.111 | C.15 |
| 1.3142 | B.112 | C.15 |
| 1.3143 | B.113 | C.15 |
| 1.3144 | B.114 | C.15 |
| 1.3145 | B.115 | C.15 |
| 1.3146 | B.116 | C.15 |
| 1.3147 | B.117 | C.15 |
| 1.3148 | B.118 | C.15 |
| 1.3149 | B.119 | C.15 |
| 1.3150 | B.120 | C.15 |
| 1.3151 | B.121 | C.15 |
| 1.3152 | B.122 | C.15 |
| 1.3153 | B.123 | C.15 |
| 1.3154 | B.124 | C.15 |
| 1.3155 | B.125 | C.15 |
| 1.3156 | B.126 | C.15 |
| 1.3157 | B.127 | C.15 |
| 1.3158 | B.128 | C.15 |
| 1.3159 | B.129 | C.15 |
| 1.3160 | B.130 | C.15 |
| 1.3161 | B.131 | C.15 |
| 1.3162 | B.132 | C.15 |
| 1.3163 | B.133 | C.15 |
| 1.3164 | B.134 | C.15 |
| 1.3165 | B.135 | C.15 |
| 1.3166 | B.136 | C.15 |
| 1.3167 | B.137 | C.15 |
| 1.3168 | B.138 | C.15 |
| 1.3169 | B.139 | C.15 |
| 1.3170 | B.140 | C.15 |
| 1.3171 | B.141 | C.15 |
| 1.3172 | B.142 | C.15 |
| 1.3173 | B.143 | C.15 |
| 1.3174 | B.144 | C.15 |
| 1.3175 | B.145 | C.15 |
| 1.3176 | B.146 | C.15 |
| 1.3177 | B.147 | C.15 |
| 1.3178 | B.148 | C.15 |
| 1.3179 | B.149 | C.15 |
| 1.3180 | B.150 | C.15 |
| 1.3181 | B.151 | C.15 |
| 1.3182 | B.152 | C.15 |
| 1.3183 | B.153 | C.15 |
| 1.3184 | B.154 | C.15 |
| 1.3185 | B.155 | C.15 |
| 1.3186 | B.156 | C.15 |
| 1.3187 | B.157 | C.15 |
| 1.3188 | B.158 | C.15 |
| 1.3189 | B.159 | C.15 |
| 1.3190 | B.160 | C.15 |
| 1.3191 | B.161 | C.15 |
| 1.3192 | B.162 | C.15 |
| 1.3193 | B.163 | C.15 |
| 1.3194 | B.164 | C.15 |
| 1.3195 | B.165 | C.15 |
| 1.3196 | B.166 | C.15 |
| 1.3197 | B.167 | C.15 |
| 1.3198 | B.168 | C.15 |
| 1.3199 | B.169 | C.15 |
| 1.3200 | B.170 | C.15 |
| 1.3201 | B.171 | C.15 |
| 1.3202 | B.172 | C.15 |
| 1.3203 | B.173 | C.15 |
| 1.3204 | B.174 | C.15 |
| 1.3205 | B.175 | C.15 |
| 1.3206 | B.176 | C.15 |
| 1.3207 | B.177 | C.15 |
| 1.3208 | B.178 | C.15 |
| 1.3209 | B.179 | C.15 |
| 1.3210 | B.180 | C.15 |
| 1.3211 | B.181 | C.15 |
| 1.3212 | B.182 | C.15 |
| 1.3213 | B.183 | C.15 |
| 1.3214 | B.184 | C.15 |
| 1.3215 | B.185 | C.15 |
| 1.3216 | B.186 | C.15 |
| 1.3217 | B.187 | C.15 |
| 1.3218 | B.188 | C.15 |
| 1.3219 | B.189 | C.15 |
| 1.3220 | B.190 | C.15 |
| 1.3221 | B.191 | C.15 |
| 1.3222 | B.192 | C.15 |
| 1.3223 | B.193 | C.15 |
| 1.3224 | B.194 | C.15 |
| 1.3225 | B.195 | C.15 |
| 1.3226 | B.196 | C.15 |
| 1.3227 | B.197 | C.15 |
| 1.3228 | B.198 | C.15 |
| 1.3229 | B.199 | C.15 |
| 1.3230 | B.200 | C.15 |
| 1.3231 | B.201 | C.15 |
| 1.3232 | B.202 | C.15 |
| 1.3233 | B.1 | C.16 |
| 1.3234 | B.2 | C.16 |
| 1.3235 | B.3 | C.16 |
| 1.3236 | B.4 | C.16 |
| 1.3237 | B.5 | C.16 |
| 1.3238 | B.6 | C.16 |
| 1.3239 | B.7 | C.16 |
| 1.3240 | B.8 | C.16 |
| 1.3241 | B.9 | C.16 |
| 1.3242 | B.10 | C.16 |
| 1.3243 | B.11 | C.16 |
| 1.3244 | B.12 | C.16 |
| 1.3245 | B.13 | C.16 |
| 1.3246 | B.14 | C.16 |
| 1.3247 | B.15 | C.16 |
| 1.3248 | B.16 | C.16 |
| 1.3249 | B.17 | C.16 |
| 1.3250 | B.18 | C.16 |
| 1.3251 | B.19 | C.16 |
| 1.3252 | B.20 | C.16 |
| 1.3253 | B.21 | C.16 |
| 1.3254 | B.22 | C.16 |
| 1.3255 | B.23 | C.16 |
| 1.3256 | B.24 | C.16 |
| 1.3257 | B.25 | C.16 |
| 1.3258 | B.26 | C.16 |
| 1.3259 | B.27 | C.16 |
| 1.3260 | B.28 | C.16 |
| 1.3261 | B.29 | C.16 |
| 1.3262 | B.30 | C.16 |
| 1.3263 | B.31 | C.16 |
| 1.3264 | B.32 | C.16 |
| 1.3265 | B.33 | C.16 |
| 1.3266 | B.34 | C.16 |
| 1.3267 | B.35 | C.16 |
| 1.3268 | B.36 | C.16 |
| 1.3269 | B.37 | C.16 |
| 1.3270 | B.38 | C.16 |
| 1.3271 | B.39 | C.16 |
| 1.3272 | B.40 | C.16 |
| 1.3273 | B.41 | C.16 |
| 1.3274 | B.42 | C.16 |
| 1.3275 | B.43 | C.16 |
| 1.3276 | B.44 | C.16 |
| 1.3277 | B.45 | C.16 |
| 1.3278 | B.46 | C.16 |
| 1.3279 | B.47 | C.16 |
| 1.3280 | B.48 | C.16 |
| 1.3281 | B.49 | C.16 |
| 1.3282 | B.50 | C.16 |
| 1.3283 | B.51 | C.16 |
| 1.3284 | B.52 | C.16 |
| 1.3285 | B.53 | C.16 |
| 1.3286 | B.54 | C.16 |
| 1.3287 | B.55 | C.16 |
| 1.3288 | B.56 | C.16 |
| 1.3289 | B.57 | C.16 |
| 1.3290 | B.58 | C.16 |
| 1.3291 | B.59 | C.16 |
| 1.3292 | B.60 | C.16 |
| 1.3293 | B.61 | C.16 |
| 1.3294 | B.62 | C.16 |
| 1.3295 | B.63 | C.16 |
| 1.3296 | B.64 | C.16 |
| 1.3297 | B.65 | C.16 |
| 1.3298 | B.66 | C.16 |
| 1.3299 | B.67 | C.16 |
| 1.3300 | B.68 | C.16 |
| 1.3301 | B.69 | C.16 |
| 1.3302 | B.70 | C.16 |
| 1.3303 | B.71 | C.16 |
| 1.3304 | B.72 | C.16 |
| 1.3305 | B.73 | C.16 |
| 1.3306 | B.74 | C.16 |
| 1.3307 | B.75 | C.16 |
| 1.3308 | B.76 | C.16 |
| 1.3309 | B.77 | C.16 |
| 1.3310 | B.78 | C.16 |
| 1.3311 | B.79 | C.16 |
| 1.3312 | B.80 | C.16 |
| 1.3313 | B.81 | C.16 |
| 1.3314 | B.82 | C.16 |
| 1.3315 | B.83 | C.16 |
| 1.3316 | B.84 | C.16 |
| 1.3317 | B.85 | C.16 |
| 1.3318 | B.86 | C.16 |
| 1.3319 | B.87 | C.16 |
| 1.3320 | B.88 | C.16 |
| 1.3321 | B.89 | C.16 |
| 1.3322 | B.90 | C.16 |
| 1.3323 | B.91 | C.16 |
| 1.3324 | B.92 | C.16 |
| 1.3325 | B.93 | C.16 |
| 1.3326 | B.94 | C.16 |
| 1.3327 | B.95 | C.16 |
| 1.3328 | B.96 | C.16 |
| 1.3329 | B.97 | C.16 |
| 1.3330 | B.98 | C.16 |
| 1.3331 | B.99 | C.16 |
| 1.3332 | B.100 | C.16 |
| 1.3333 | B.101 | C.16 |
| 1.3334 | B.102 | C.16 |
| 1.3335 | B.103 | C.16 |
| 1.3336 | B.104 | C.16 |
| 1.3337 | B.105 | C.16 |
| 1.3338 | B.106 | C.16 |
| 1.3339 | B.107 | C.16 |
| 1.3340 | B.108 | C.16 |
| 1.3341 | B.109 | C.16 |
| 1.3342 | B.110 | C.16 |
| 1.3343 | B.111 | C.16 |
| 1.3344 | B.112 | C.16 |
| 1.3345 | B.113 | C.16 |
| 1.3346 | B.114 | C.16 |
| 1.3347 | B.115 | C.16 |
| 1.3348 | B.116 | C.16 |
| 1.3349 | B.117 | C.16 |
| 1.3350 | B.118 | C.16 |
| 1.3351 | B.119 | C.16 |
| 1.3352 | B.120 | C.16 |
| 1.3353 | B.121 | C.16 |
| 1.3354 | B.122 | C.16 |
| 1.3355 | B.123 | C.16 |
| 1.3356 | B.124 | C.16 |
| 1.3357 | B.125 | C.16 |
| 1.3358 | B.126 | C.16 |
| 1.3359 | B.127 | C.16 |
| 1.3360 | B.128 | C.16 |
| 1.3361 | B.129 | C.16 |
| 1.3362 | B.130 | C.16 |
| 1.3363 | B.131 | C.16 |
| 1.3364 | B.132 | C.16 |
| 1.3365 | B.133 | C.16 |
| 1.3366 | B.134 | C.16 |
| 1.3367 | B.135 | C.16 |
| 1.3368 | B.136 | C.16 |
| 1.3369 | B.137 | C.16 |
| 1.3370 | B.138 | C.16 |
| 1.3371 | B.139 | C.16 |
| 1.3372 | B.140 | C.16 |
| 1.3373 | B.141 | C.16 |
| 1.3374 | B.142 | C.16 |
| 1.3375 | B.143 | C.16 |
| 1.3376 | B.144 | C.16 |
| 1.3377 | B.145 | C.16 |
| 1.3378 | B.146 | C.16 |
| 1.3379 | B.147 | C.16 |
| 1.3380 | B.148 | C.16 |
| 1.3381 | B.149 | C.16 |
| 1.3382 | B.150 | C.16 |
| 1.3383 | B.151 | C.16 |
| 1.3384 | B.152 | C.16 |
| 1.3385 | B.153 | C.16 |
| 1.3386 | B.154 | C.16 |
| 1.3387 | B.155 | C.16 |
| 1.3388 | B.156 | C.16 |
| 1.3389 | B.157 | C.16 |
| 1.3390 | B.158 | C.16 |
| 1.3391 | B.159 | C.16 |
| 1.3392 | B.160 | C.16 |
| 1.3393 | B.161 | C.16 |
| 1.3394 | B.162 | C.16 |
| 1.3395 | B.163 | C.16 |
| 1.3396 | B.164 | C.16 |
| 1.3397 | B.165 | C.16 |
| 1.3398 | B.166 | C.16 |
| 1.3399 | B.167 | C.16 |
| 1.3400 | B.168 | C.16 |
| 1.3401 | B.169 | C.16 |
| 1.3402 | B.170 | C.16 |
| 1.3403 | B.171 | C.16 |
| 1.3404 | B.172 | C.16 |
| 1.3405 | B.173 | C.16 |
| 1.3406 | B.174 | C.16 |
| 1.3407 | B.175 | C.16 |
| 1.3408 | B.176 | C.16 |
| 1.3409 | B.177 | C.16 |
| 1.3410 | B.178 | C.16 |
| 1.3411 | B.179 | C.16 |
| 1.3412 | B.180 | C.16 |
| 1.3413 | B.181 | C.16 |
| 1.3414 | B.182 | C.16 |
| 1.3415 | B.183 | C.16 |
| 1.3416 | B.184 | C.16 |
| 1.3417 | B.185 | C.16 |
| 1.3418 | B.186 | C.16 |
| 1.3419 | B.187 | C.16 |
| 1.3420 | B.188 | C.16 |
| 1.3421 | B.189 | C.16 |
| 1.3422 | B.190 | C.16 |
| 1.3423 | B.191 | C.16 |
| 1.3424 | B.192 | C.16 |
| 1.3425 | B.193 | C.16 |
| 1.3426 | B.194 | C.16 |
| 1.3427 | B.195 | C.16 |
| 1.3428 | B.196 | C.16 |
| 1.3429 | B.197 | C.16 |
| 1.3430 | B.198 | C.16 |
| 1.3431 | B.199 | C.16 |
| 1.3432 | B.200 | C.16 |
| 1.3433 | B.201 | C.16 |
| 1.3434 | B.202 | C.16 |
| 1.3435 | B.1 | C.17 |
| 1.3436 | B.2 | C.17 |
| 1.3437 | B.3 | C.17 |
| 1.3438 | B.4 | C.17 |
| 1.3439 | B.5 | C.17 |
| 1.3440 | B.6 | C.17 |
| 1.3441 | B.7 | C.17 |
| 1.3442 | B.8 | C.17 |
| 1.3443 | B.9 | C.17 |
| 1.3444 | B.10 | C.17 |
| 1.3445 | B.11 | C.17 |
| 1.3446 | B.12 | C.17 |
| 1.3447 | B.13 | C.17 |
| 1.3448 | B.14 | C.17 |
| 1.3449 | B.15 | C.17 |
| 1.3450 | B.16 | C.17 |
| 1.3451 | B.17 | C.17 |
| 1.3452 | B.18 | C.17 |
| 1.3453 | B.19 | C.17 |
| 1.3454 | B.20 | C.17 |
| 1.3455 | B.21 | C.17 |
| 1.3456 | B.22 | C.17 |
| 1.3457 | B.23 | C.17 |
| 1.3458 | B.24 | C.17 |
| 1.3459 | B.25 | C.17 |
| 1.3460 | B.26 | C.17 |
| 1.3461 | B.27 | C.17 |
| 1.3462 | B.28 | C.17 |
| 1.3463 | B.29 | C.17 |
| 1.3464 | B.30 | C.17 |
| 1.3465 | B.31 | C.17 |
| 1.3466 | B.32 | C.17 |
| 1.3467 | B.33 | C.17 |
| 1.3468 | B.34 | C.17 |
| 1.3469 | B.35 | C.17 |
| 1.3470 | B.36 | C.17 |
| 1.3471 | B.37 | C.17 |
| 1.3472 | B.38 | C.17 |
| 1.3473 | B.39 | C.17 |
| 1.3474 | B.40 | C.17 |
| 1.3475 | B.41 | C.17 |
| 1.3476 | B.42 | C.17 |
| 1.3477 | B.43 | C.17 |
| 1.3478 | B.44 | C.17 |
| 1.3479 | B.45 | C.17 |
| 1.3480 | B.46 | C.17 |
| 1.3481 | B.47 | C.17 |
| 1.3482 | B.48 | C.17 |
| 1.3483 | B.49 | C.17 |
| 1.3484 | B.50 | C.17 |
| 1.3485 | B.51 | C.17 |
| 1.3486 | B.52 | C.17 |
| 1.3487 | B.53 | C.17 |
| 1.3488 | B.54 | C.17 |
| 1.3489 | B.55 | C.17 |
| 1.3490 | B.56 | C.17 |
| 1.3491 | B.57 | C.17 |
| 1.3492 | B.58 | C.17 |
| 1.3493 | B.59 | C.17 |
| 1.3494 | B.60 | C.17 |
| 1.3495 | B.61 | C.17 |
| 1.3496 | B.62 | C.17 |
| 1.3497 | B.63 | C.17 |
| 1.3498 | B.64 | C.17 |
| 1.3499 | B.65 | C.17 |
| 1.3500 | B.66 | C.17 |
| 1.3501 | B.67 | C.17 |
| 1.3502 | B.68 | C.17 |
| 1.3503 | B.69 | C.17 |
| 1.3504 | B.70 | C.17 |
| 1.3505 | B.71 | C.17 |
| 1.3506 | B.72 | C.17 |
| 1.3507 | B.73 | C.17 |
| 1.3508 | B.74 | C.17 |
| 1.3509 | B.75 | C.17 |
| 1.3510 | B.76 | C.17 |
| 1.3511 | B.77 | C.17 |
| 1.3512 | B.78 | C.17 |
| 1.3513 | B.79 | C.17 |
| 1.3514 | B.80 | C.17 |
| 1.3515 | B.81 | C.17 |
| 1.3516 | B.82 | C.17 |
| 1.3517 | B.83 | C.17 |
| 1.3518 | B.84 | C.17 |
| 1.3519 | B.85 | C.17 |
| 1.3520 | B.86 | C.17 |
| 1.3521 | B.87 | C.17 |
| 1.3522 | B.88 | C.17 |
| 1.3523 | B.89 | C.17 |
| 1.3524 | B.90 | C.17 |
| 1.3525 | B.91 | C.17 |
| 1.3526 | B.92 | C.17 |
| 1.3527 | B.93 | C.17 |
| 1.3528 | B.94 | C.17 |
| 1.3529 | B.95 | C.17 |
| 1.3530 | B.96 | C.17 |
| 1.3531 | B.97 | C.17 |
| 1.3532 | B.98 | C.17 |
| 1.3533 | B.99 | C.17 |
| 1.3534 | B.100 | C.17 |
| 1.3535 | B.101 | C.17 |
| 1.3536 | B.102 | C.17 |
| 1.3537 | B.103 | C.17 |
| 1.3538 | B.104 | C.17 |
| 1.3539 | B.105 | C.17 |
| 1.3540 | B.106 | C.17 |
| 1.3541 | B.107 | C.17 |
| 1.3542 | B.108 | C.17 |
| 1.3543 | B.109 | C.17 |
| 1.3544 | B.110 | C.17 |
| 1.3545 | B.111 | C.17 |
| 1.3546 | B.112 | C.17 |
| 1.3547 | B.113 | C.17 |
| 1.3548 | B.114 | C.17 |
| 1.3549 | B.115 | C.17 |
| 1.3550 | B.116 | C.17 |
| 1.3551 | B.117 | C.17 |
| 1.3552 | B.118 | C.17 |
| 1.3553 | B.119 | C.17 |
| 1.3554 | B.120 | C.17 |
| 1.3555 | B.121 | C.17 |
| 1.3556 | B.122 | C.17 |
| 1.3557 | B.123 | C.17 |
| 1.3558 | B.124 | C.17 |
| 1.3559 | B.125 | C.17 |
| 1.3560 | B.126 | C.17 |
| 1.3561 | B.127 | C.17 |
| 1.3562 | B.128 | C.17 |
| 1.3563 | B.129 | C.17 |
| 1.3564 | B.130 | C.17 |
| 1.3565 | B.131 | C.17 |
| 1.3566 | B.132 | C.17 |
| 1.3567 | B.133 | C.17 |
| 1.3568 | B.134 | C.17 |
| 1.3569 | B.135 | C.17 |
| 1.3570 | B.136 | C.17 |
| 1.3571 | B.137 | C.17 |
| 1.3572 | B.138 | C.17 |
| 1.3573 | B.139 | C.17 |
| 1.3574 | B.140 | C.17 |
| 1.3575 | B.141 | C.17 |
| 1.3576 | B.142 | C.17 |
| 1.3577 | B.143 | C.17 |
| 1.3578 | B.144 | C.17 |
| 1.3579 | B.145 | C.17 |
| 1.3580 | B.146 | C.17 |
| 1.3581 | B.147 | C.17 |
| 1.3582 | B.148 | C.17 |
| 1.3583 | B.149 | C.17 |
| 1.3584 | B.150 | C.17 |
| 1.3585 | B.151 | C.17 |
| 1.3586 | B.152 | C.17 |
| 1.3587 | B.153 | C.17 |
| 1.3588 | B.154 | C.17 |
| 1.3589 | B.155 | C.17 |
| 1.3590 | B.156 | C.17 |
| 1.3591 | B.157 | C.17 |
| 1.3592 | B.158 | C.17 |
| 1.3593 | B.159 | C.17 |
| 1.3594 | B.160 | C.17 |
| 1.3595 | B.161 | C.17 |
| 1.3596 | B.162 | C.17 |
| 1.3597 | B.163 | C.17 |
| 1.3598 | B.164 | C.17 |
| 1.3599 | B.165 | C.17 |
| 1.3600 | B.166 | C.17 |
| 1.3601 | B.167 | C.17 |
| 1.3602 | B.168 | C.17 |
| 1.3603 | B.169 | C.17 |
| 1.3604 | B.170 | C.17 |
| 1.3605 | B.171 | C.17 |
| 1.3606 | B.172 | C.17 |
| 1.3607 | B.173 | C.17 |
| 1.3608 | B.174 | C.17 |
| 1.3609 | B.175 | C.17 |
| 1.3610 | B.176 | C.17 |
| 1.3611 | B.177 | C.17 |
| 1.3612 | B.178 | C.17 |
| 1.3613 | B.179 | C.17 |
| 1.3614 | B.180 | C.17 |
| 1.3615 | B.181 | C.17 |
| 1.3616 | B.182 | C.17 |
| 1.3617 | B.183 | C.17 |
| 1.3618 | B.184 | C.17 |
| 1.3619 | B.185 | C.17 |
| 1.3620 | B.186 | C.17 |
| 1.3621 | B.187 | C.17 |
| 1.3622 | B.188 | C.17 |
| 1.3623 | B.189 | C.17 |
| 1.3624 | B.190 | C.17 |
| 1.3625 | B.191 | C.17 |
| 1.3626 | B.192 | C.17 |
| 1.3627 | B.193 | C.17 |
| 1.3628 | B.194 | C.17 |
| 1.3629 | B.195 | C.17 |
| 1.3630 | B.196 | C.17 |
| 1.3631 | B.197 | C.17 |
| 1.3632 | B.198 | C.17 |
| 1.3633 | B.199 | C.17 |
| 1.3634 | B.200 | C.17 |
| 1.3635 | B.201 | C.17 |
| 1.3636 | B.202 | C.17 |
| 1.3637 | — | C.1 |
| 1.3638 | — | C.2 |
| 1.3639 | — | C.3 |
| 1.3640 | — | C.4 |
| 1.3641 | — | C.5 |
| 1.3642 | — | C.6 |
| 1.3643 | — | C.7 |
| 1.3644 | — | C.8 |
| 1.3645 | — | C.9 |
| 1.3646 | — | C.10 |
| 1.3647 | — | C.11 |
| 1.3648 | — | C.12 |
| 1.3649 | — | C.13 |
| 1.3650 | — | C.14 |
| 1.3651 | — | C.15 |
| 1.3652 | — | C.16 |
| 1.3653 | — | C.17 |

The specific number for each single composition is deductible as follows:

Composition 1.203 e.g. comprises the compound (1.1.I-25), clethodim (B.1) and benoxacor (0.1) (see table B, entry B.1 and table 0, entry 0.1).

Also especially preferred are compositions 2.1 to 2.3653 which differ from the corresponding compositions 1.1 to 1.3653 only in that they comprise the compound (1.1.I-4) in place of the compound (1.1.I-3).

Also especially preferred are compositions 3.1 to 3.3653 which differ from the corresponding compositions 1.1 to 1.3653 only in that they additionally comprise B.2 as further herbicide B.

Also especially preferred are compositions 4.1 to 4.3653 which differ from the corresponding compositions 1.1 to 1.3653 only in that they additionally comprise B.8 as further herbicide B.

Also especially preferred are compositions 5.1 to 5.3653 which differ from the corresponding compositions 1.1 to 1.3653 only in that they additionally comprise B.30 as further herbicide B.

Also especially preferred are compositions 6.1 to 6.3653 which differ from the corresponding compositions 1.1 to 1.3653 only in that they additionally comprise B.32 as further herbicide B.

Also especially preferred are compositions 7.1 to 7.3653 which differ from the corresponding compositions 1.1 to 1.3653 only in that they additionally comprise B.35 as further herbicide B.

Also especially preferred are compositions 8.1 to 8.3653 which differ from the corresponding compositions 1.1 to 1.3653 only in that they additionally comprise B.38 as further herbicide B.

Also especially preferred are compositions 9.1 to 9.3653 which differ from the corresponding compositions 1.1 to 1.3653 only in that they additionally comprise B.40 as further herbicide B.

Also especially preferred are compositions 10.1 to 10.3653 which differ from the corresponding compositions 1.1 to 1.3653 only in that they additionally comprise B.51 as further herbicide B.

Also especially preferred are compositions 11.1 to 11.3653 which differ from the corresponding compositions 1.1 to 1.3653 only in that they additionally comprise B.55 as further herbicide B.

Also especially preferred are compositions 12.1 to 12.3653 which differ from the corresponding compositions 1.1 to 1.3653 only in that they additionally comprise B.56 as further herbicide B.

Also especially preferred are compositions 13.1 to 13.3653 which differ from the corresponding compositions 1.1 to 1.3653 only in that they additionally comprise B.64 as further herbicide B.

Also especially preferred are compositions 14.1 to 14.3653 which differ from the corresponding compositions 1.1 to 1.3653 only in that they additionally comprise B.66 as further herbicide B.

Also especially preferred are compositions 15.1 to 15.3653 which differ from the corresponding compositions 1.1 to 1.3653 only in that they additionally comprise B.67 as further herbicide B.

Also especially preferred are compositions 16.1 to 16.3653 which differ from the corresponding compositions 1.1 to 1.3653 only in that they additionally comprise B.68 as further herbicide B.

Also especially preferred are compositions 17.1 to 17.3653 which differ from the corresponding compositions 1.1 to 1.3653 only in that they additionally comprise B.69 as further herbicide B.

Also especially preferred are compositions 18.1 to 18.3653 which differ from the corresponding compositions 1.1 to 1.3653 only in that they additionally comprise B.73 as further herbicide B.

Also especially preferred are compositions 19.1 to 19.3653 which differ from the corresponding compositions 1.1 to 1.3653 only in that they additionally comprise B.76 as further herbicide B.

Also especially preferred are compositions 20.1 to 20.3653 which differ from the corresponding compositions 1.1 to 1.3653 only in that they additionally comprise B.81 as further herbicide B.

Also especially preferred are compositions 21.1 to 21.3653 which differ from the corresponding compositions 1.1 to 1.3653 only in that they additionally comprise B.82 as further herbicide B.

Also especially preferred are compositions 22.1 to 22.3653 which differ from the corresponding compositions 1.1 to 1.3653 only in that they additionally comprise B.85 as further herbicide B.

Also especially preferred are compositions 23.1 to 23.3653 which differ from the corresponding compositions 1.1 to 1.3653 only in that they additionally comprise B.88 as further herbicide B.

Also especially preferred are compositions 24.1 to 24.3653 which differ from the corresponding compositions 1.1 to 1.3653 only in that they additionally comprise B.89 as further herbicide B.

Also especially preferred are compositions 25.1 to 25.3653 which differ from the corresponding compositions 1.1 to 1.3653 only in that they additionally comprise B.94 as further herbicide B.

Also especially preferred are compositions 26.1 to 26.3653 which differ from the corresponding compositions 1.1 to 1.3653 only in that they additionally comprise B.95 as further herbicide B.

Also especially preferred are compositions 27.1 to 27.3653 which differ from the corresponding compositions 1.1 to 1.3653 only in that they additionally comprise B.98 as further herbicide B.

Also especially preferred are compositions 28.1 to 28.3653 which differ from the corresponding compositions 1.1 to 1.3653 only in that they additionally comprise B.100 as further herbicide B.

Also especially preferred are compositions 29.1 to 29.3653 which differ from the corresponding compositions 1.1 to 1.3653 only in that they additionally comprise B.103 as further herbicide B.

Also especially preferred are compositions 30.1 to 30.3653 which differ from the corresponding compositions 1.1 to 1.3653 only in that they additionally comprise B.103 and B.67 as further herbicides B.

Also especially preferred are compositions 31.1 to 31.3653 which differ from the corresponding compositions 1.1 to 1.3653 only in that they additionally comprise B.103 and B.76 as further herbicides B.

Also especially preferred are compositions 32.1 to 32.3653 which differ from the corresponding compositions 1.1 to 1.3653 only in that they additionally comprise B.103 and B.82 as further herbicides B.

Also especially preferred are compositions 33.1 to 33.3653 which differ from the corresponding compositions 1.1 to 1.3653 only in that they additionally comprise B.104 as further herbicide B.

Also especially preferred are compositions 34.1 to 34.3653 which differ from the corresponding compositions 1.1 to 1.3653 only in that they additionally comprise B.104 and B.67 as further herbicides B.

Also especially preferred are compositions 35.1 to 35.3653 which differ from the corresponding compositions 1.1 to 1.3653 only in that they additionally comprise B.104 and B.76 as further herbicides B.

Also especially preferred are compositions 36.1 to 36.3653 which differ from the corresponding compositions 1.1 to 1.3653 only in that they additionally comprise B.104 and B.82 as further herbicides B.

Also especially preferred are compositions 37.1 to 37.3653 which differ from the corresponding compositions 1.1 to 1.3653 only in that they additionally comprise B.106 as further herbicide B.

Also especially preferred are compositions 38.1 to 38.3653 which differ from the corresponding compositions 1.1 to 1.3653 only in that they additionally comprise B.107 as further herbicide B.

Also especially preferred are compositions 39.1 to 39.3653 which differ from the corresponding compositions 1.1 to 1.3653 only in that they additionally comprise B. 107 and B.67 as further herbicides B.

Also especially preferred are compositions 40.1 to 40.3653 which differ from the corresponding compositions 1.1 to 1.3653 only in that they additionally comprise B. 107 and B.76 as further herbicides B.

Also especially preferred are compositions 41.1 to 41.3653 which differ from the corresponding compositions 1.1 to 1.3653 only in that they additionally comprise B. 107 and B.82 as further herbicides B.

Also especially preferred are compositions 42.1 to 42.3653 which differ from the corresponding compositions 1.1 to 1.3653 only in that they additionally comprise B.109 as further herbicide B.

Also especially preferred are compositions 43.1 to 43.3653 which differ from the corresponding compositions 1.1 to 1.3653 only in that they additionally comprise B.111 as further herbicide B.

Also especially preferred are compositions 44.1 to 44.3653 which differ from the corresponding compositions 1.1 to 1.3653 only in that they additionally comprise B.111 and B.67 as further herbicides B.

Also especially preferred are compositions 45.1 to 45.3653 which differ from the corresponding compositions 1.1 to 1.3653 only in that they additionally comprise B.111 and B.76 as further herbicides B.

Also especially preferred are compositions 46.1 to 46.3653 which differ from the corresponding compositions 1.1 to 1.3653 only in that they additionally comprise B.111 and B.82 as further herbicides B.

Also especially preferred are compositions 47.1 to 47.3653 which differ from the corresponding compositions 1.1 to 1.3653 only in that they additionally comprise B. 116 as further herbicide B.

Also especially preferred are compositions 48.1 to 48.3653 which differ from the corresponding compositions 1.1 to 1.3653 only in that they additionally comprise B.116 and B.67 as further herbicides B.

Also especially preferred are compositions 49.1 to 49.3653 which differ from the corresponding compositions 1.1 to 1.3653 only in that they additionally comprise B.116 and B.94 as further herbicides B.

Also especially preferred are compositions 50.1 to 50.3653 which differ from the corresponding compositions 1.1 to 1.3653 only in that they additionally comprise B.116 and B.103 as further herbicides B.

Also especially preferred are compositions 51.1 to 51.3653 which differ from the corresponding compositions 1.1 to 1.3653 only in that they additionally comprise B.116 and B.128 as further herbicides B.

Also especially preferred are compositions 52.1 to 52.3653 which differ from the corresponding compositions 1.1 to 1.3653 only in that they additionally comprise B.116 and B.104 as further herbicides B.

Also especially preferred are compositions 53.1 to 53.3653 which differ from the corresponding compositions 1.1 to 1.3653 only in that they additionally comprise B.116 and B.107 as further herbicides B.

Also especially preferred are compositions 54.1 to 54.3653 which differ from the corresponding compositions 1.1 to 1.3653 only in that they additionally comprise B.116 and B.111 as further herbicides B.

Also especially preferred are compositions 55.1 to 55.3653 which differ from the corresponding compositions 1.1 to 1.3653 only in that they additionally comprise B.122 as further herbicide B.

Also especially preferred are compositions 56.1 to 56.3653 which differ from the corresponding compositions 1.1 to 1.3653 only in that they additionally comprise B.126 as further herbicide B.

Also especially preferred are compositions 57.1 to 57.3653 which differ from the corresponding compositions 1.1 to 1.3653 only in that they additionally comprise B.128 as further herbicide B.

Also especially preferred are compositions 58.1 to 58.3653 which differ from the corresponding compositions 1.1 to 1.3653 only in that they additionally comprise B.131 as further herbicide B.

Also especially preferred are compositions 59.1 to 59.3653 which differ from the corresponding compositions 1.1 to 1.3653 only in that they additionally comprise B.132 as further herbicide B.

Also especially preferred are compositions 60.1 to 60.3653 which differ from the corresponding compositions 1.1 to 1.3653 only in that they additionally comprise B.133 as further herbicide B.

Also especially preferred are compositions 61.1 to 61.3653 which differ from the corresponding compositions 1.1 to 1.3653 only in that they additionally comprise B.135 as further herbicide B.

Also especially preferred are compositions 62.1 to 62.3653 which differ from the corresponding compositions 1.1 to 1.3653 only in that they additionally comprise B.137 as further herbicide B.

Also especially preferred are compositions 63.1 to 63.3653 which differ from the corresponding compositions 1.1 to 1.3653 only in that they additionally comprise B.138 as further herbicide B.

Also especially preferred are compositions 64.1 to 64.3653 which differ from the corresponding compositions 1.1 to 1.3653 only in that they additionally comprise B.140 as further herbicide B.

Also especially preferred are compositions 65.1 to 65.3653 which differ from the corresponding compositions 1.1 to 1.3653 only in that they additionally comprise B.145 as further herbicide B.

Also especially preferred are compositions 66.1 to 66.3653 which differ from the corresponding compositions 1.1 to 1.3653 only in that they additionally comprise B.153 as further herbicide B.

Also especially preferred are compositions 67.1 to 67.3653 which differ from the corresponding compositions 1.1 to 1.3653 only in that they additionally comprise B.156 as further herbicide B.

Also especially preferred are compositions 68.1 to 68.3653 which differ from the corresponding compositions 1.1 to 1.3653 only in that they additionally comprise B.171 as further herbicide B.

Also especially preferred are compositions 69.1 to 69.3653 which differ from the corresponding compositions 1.1 to 1.3653 only in that they additionally comprise B.174 as further herbicide B.

Also especially preferred are compositions 70.1 to 70.3653 which differ from the corresponding compositions 1.1 to 1.3653 only in that they comprise the compound (1.1.I-10) in place of the compound (1.1.I-3).

Also especially preferred are compositions 71.1 to 71.3653 which differ from the corresponding compositions 1.1 to 1.3653 only in that they comprise the compound (1.1.I-11) in place of the compound (1.1.I-3).

Also especially preferred are compositions 72.1 to 72.3653 which differ from the corresponding compositions 1.1 to 1.3653 only in that they comprise the compound (1.1.I-17) in place of the compound (1.1.I-3).

Also especially preferred are compositions 73.1 to 73.3653 which differ from the corresponding compositions 1.1 to 1.3653 only in that they comprise the compound (1.1.I-18) in place of the compound (1.1.I-3).

Also especially preferred are compositions 74.1 to 74.3653 which differ from the corresponding compositions 1.1 to 1.3653 only in that they comprise the compound (1.1.I-24) in place of the compound (1.1.I-3).

Also especially preferred are compositions 75.1 to 75.3653 which differ from the corresponding compositions 1.1 to 1.3653 only in that they comprise the compound (1.1.I-25) in place of the compound (1.1.I-3).

Also especially preferred are compositions 76.1 to 76.3653 which differ from the corresponding compositions 1.1 to 1.3653 only in that they comprise the compound (1.1.I-31) in place of the compound (1.1.I-3).

Also especially preferred are compositions 77.1 to 77.3653 which differ from the corresponding compositions 1.1 to 1.3653 only in that they comprise the compound (1.1.I-32) in place of the compound (1.1.I-3).

Also especially preferred are compositions 78.1 to 78.3653 which differ from the corresponding compositions 1.1 to 1.3653 only in that they comprise the compound (1.1.I-38) in place of the compound (1.1.I-3).

Also especially preferred are compositions 79.1 to 79.3653 which differ from the corresponding compositions 1.1 to 1.3653 only in that they comprise the compound (1.1.I-39) in place of the compound (1.1.I-3).

Also especially preferred are compositions 80.1 to 80.3653 which differ from the corresponding compositions 1.1 to 1.3653 only in that they comprise the compound (1.1.I-45) in place of the compound (1.1.I-3).

Also especially preferred are compositions 81.1 to 81.3653 which differ from the corresponding compositions 1.1 to 1.3653 only in that they comprise the compound (1.1.I-46) in place of the compound (1.1.I-3).

Also especially preferred are compositions 82.1 to 82.3653 which differ from the corresponding compositions 1.1 to 1.3653 only in that they comprise the compound (1.1.I-52) in place of the compound (1.1.I-3).

Also especially preferred are compositions 83.1 to 83.3653 which differ from the corresponding compositions 1.1 to 1.3653 only in that they comprise the compound (1.1.I-53) in place of the compound (1.1.I-3).

Also especially preferred are compositions 84.1 to 84.3653 which differ from the corresponding compositions 1.1 to 1.3653 only in that they comprise the compound (1.1.I-59) in place of the compound (1.1.I-3).

Also especially preferred are compositions 85.1 to 85.3653 which differ from the corresponding compositions 1.1 to 1.3653 only in that they comprise the compound (1.1.I-60) in place of the compound (1.1.I-3).

Also especially preferred are compositions 86.1 to 86.3653 which differ from the corresponding compositions 1.1 to 1.3653 only in that they comprise the compound (1.1.I-66) in place of the compound (1.1.I-3).

Also especially preferred are compositions 87.1 to 87.3653 which differ from the corresponding compositions 1.1 to 1.3653 only in that they comprise the compound (1.1.I-67) in place of the compound (1.1.I-3).

Also especially preferred are compositions 88.1 to 88.3653 which differ from the corresponding compositions 1.1 to 1.3653 only in that they comprise the compound (1.2.I-3) in place of the compound (1.1.I-3).

Also especially preferred are compositions 89.1 to 89.3653 which differ from the corresponding compositions 1.1 to 1.3653 only in that they comprise the compound (1.2.I-4) in place of the compound (1.1.I-3).

Also especially preferred are compositions 90.1 to 90.3653 which differ from the corresponding compositions 1.1 to 1.3653 only in that they comprise the compound (1.2.I-10) in place of the compound (1.1.I-3).

Also especially preferred are compositions 91.1 to 91.3653 which differ from the corresponding compositions 1.1 to 1.3653 only in that they comprise the compound (1.2.I-11) in place of the compound (1.1.I-3).

Also especially preferred are compositions 92.1 to 92.3653 which differ from the corresponding compositions 1.1 to 1.3653 only in that they comprise the compound (1.2.I-17) in place of the compound (1.1.I-3).

Also especially preferred are compositions 93.1 to 93.3653 which differ from the corresponding compositions 1.1 to 1.3653 only in that they comprise the compound (1.2.I-24) in place of the compound (1.1.I-3).

Also especially preferred are compositions 94.1 to 94.3653 which differ from the corresponding compositions 1.1 to 1.3653 only in that they comprise the compound (1.2.I-25) in place of the compound (1.1.I-3).

Also especially preferred are compositions 95.1 to 95.3653 which differ from the corresponding compositions 1.1 to 1.3653 only in that they comprise the compound (1.2.I-31) in place of the compound (1.1.I-3).

Also especially preferred are compositions 96.1 to 96.3653 which differ from the corresponding compositions 1.1 to 1.3653 only in that they comprise the compound (1.2.I-32) in place of the compound (1.1.I-3).

Also especially preferred are compositions 97.1 to 97.3653 which differ from the corresponding compositions 1.1 to 1.3653 only in that they comprise the compound (1.2.I-38) in place of the compound (1.1.I-3).

Also especially preferred are compositions 98.1 to 98.3653 which differ from the corresponding compositions 1.1 to 1.3653 only in that they comprise the compound (1.2.I-39) in place of the compound (1.1.I-3).

Also especially preferred are compositions 99.1 to 99.3653 which differ from the corresponding compositions 1.1 to 1.3653 only in that they comprise the compound (1.2.I-45) in place of the compound (1.1.I-3).

Also especially preferred are compositions 100.1 to 100.3653 which differ from the corresponding compositions 1.1 to 1.3653 only in that they comprise the compound (1.2.I-46) in place of the compound (1.1.I-3).

Also especially preferred are compositions 101.1 to 101.3653 which differ from the corresponding compositions 1.1 to 1.3653 only in that they comprise the compound (1.2.I-52) in place of the compound (1.1.I-3).

Also especially preferred are compositions 102.1 to 102.3653 which differ from the corresponding compositions 1.1 to 1.3653 only in that they comprise the compound (1.2.I-53) in place of the compound (1.1.I-3).

Also especially preferred are compositions 103.1 to 103.3653 which differ from the corresponding compositions 1.1 to 1.3653 only in that they comprise the compound (1.2.I-59) in place of the compound (1.1.I-3).

Also especially preferred are compositions 104.1 to 104.3653 which differ from the corresponding compositions 1.1 to 1.3653 only in that they comprise the compound (1.2.I-60) in place of the compound (1.1.I-3).

Also especially preferred are compositions 105.1 to 105.3653 which differ from the corresponding compositions 1.1 to 1.3653 only in that they comprise the compound (1.2.I-66) in place of the compound (1.1.I-3).

Also especially preferred are compositions 106.1 to 106.3653 which differ from the corresponding compositions 1.1 to 1.3653 only in that they comprise the compound (1.2.I-67) in place of the compound (1.1.I-3).

Also especially preferred are compositions 107.1 to 107.3653 which differ from the corresponding compositions 1.1 to 1.3653 only in that they comprise the compound (1.5.I-3) in place of the compound (1.1.I-3).

Also especially preferred are compositions 108.1 to 108.3653 which differ from the corresponding compositions 1.1 to 1.3653 only in that they comprise the compound (1.5.I-4) in place of the compound (1.1.I-3).

Also especially preferred are compositions 109.1 to 109.3653 which differ from the corresponding compositions 1.1 to 1.3653 only in that they comprise the compound (1.5.I-10) in place of the compound (1.1.I-3).

Also especially preferred are compositions 110.1 to 110.3653 which differ from the corresponding compositions 1.1 to 1.3653 only in that they comprise the compound (1.5.I-11) in place of the compound (1.1.I-3).

Also especially preferred are compositions 111.1 to 111.3653 which differ from the corresponding compositions 1.1 to 1.3653 only in that they comprise the compound (1.5.I-17) in place of the compound (1.1.I-3).

Also especially preferred are compositions 112.1 to 112.3653 which differ from the corresponding compositions 1.1 to 1.3653 only in that they comprise the compound (1.5.I-18) in place of the compound (1.1.I-3).

Also especially preferred are compositions 113.1 to 113.3653 which differ from the corresponding compositions 1.1 to 1.3653 only in that they comprise the compound (1.5.I-24) in place of the compound (1.1.I-3).

Also especially preferred are compositions 114.1 to 114.3653 which differ from the corresponding compositions 1.1 to 1.3653 only in that they comprise the compound (1.5.I-25) in place of the compound (1.1.I-3).

Also especially preferred are compositions 115.1 to 115.3653 which differ from the corresponding compositions 1.1 to 1.3653 only in that they comprise the compound (1.5.I-31) in place of the compound (1.1.I-3).

Also especially preferred are compositions 116.1 to 116.3653 which differ from the corresponding compositions 1.1 to 1.3653 only in that they comprise the compound (1.5.I-32) in place of the compound (1.1.I-3).

Also especially preferred are compositions 117.1 to 117.3653 which differ from the corresponding compositions 1.1 to 1.3653 only in that they comprise the compound (1.5.I-38) in place of the compound (1.1.I-3).

Also especially preferred are compositions 118.1 to 118.3653 which differ from the corresponding compositions 1.1 to 1.3653 only in that they comprise the compound (1.5.I-39) in place of the compound (1.1.I-3).

Also especially preferred are compositions 119.1 to 119.3653 which differ from the corresponding compositions 1.1 to 1.3653 only in that they comprise the compound (1.5.I-45) in place of the compound (1.1.I-3).

Also especially preferred are compositions 120.1 to 120.3653 which differ from the corresponding compositions 1.1 to 1.3653 only in that they comprise the compound (1.5.I-46) in place of the compound (1.1.I-3).

Also especially preferred are compositions 121.1 to 121.3653 which differ from the corresponding compositions 1.1 to 1.3653 only in that they comprise the compound (1.5.I-52) in place of the compound (1.1.I-3).

Also especially preferred are compositions 122.1 to 122.3653 which differ from the corresponding compositions 1.1 to 1.3653 only in that they comprise the compound (1.5.I-53) in place of the compound (1.1.I-3).

Also especially preferred are compositions 123.1 to 123.3653 which differ from the corresponding compositions 1.1 to 1.3653 only in that they comprise the compound (1.5.I-59) in place of the compound (1.1.I-3).

Also especially preferred are compositions 124.1 to 124.3653 which differ from the corresponding compositions 1.1 to 1.3653 only in that they comprise the compound (1.5.I-60) in place of the compound (1.1.I-3).

Also especially preferred are compositions 125.1 to 125.3653 which differ from the corresponding compositions 1.1 to 1.3653 only in that they comprise the compound (1.5.I-66) in place of the compound (1.1.I-3).

Also especially preferred are compositions 126.1 to 126.3653 which differ from the corresponding compositions 1.1 to 1.3653 only in that they comprise the compound (1.5.I-67) in place of the compound (1.1.I-3).

Also especially preferred are compositions 127.1 to 127.3653 which differ from the corresponding compositions 1.1 to 1.3653 only in that they comprise the compound (1.2.I-18) in place of the compound (1.1.I-3).

The invention also relates to agrochemical compositions comprising at least an auxiliary and at least one pyrimidine compound of formula (I) according to the invention.

An agrochemical composition comprises a pesticidal effective amount of a pyrimidine compound of formula (I). The term “effective amount” denotes an amount of the composition or of the compounds I, which is sufficient for controlling unwanted plants, especially for controlling unwanted plants in cultivated plants and which does not result in a substantial damage to the treated plants. Such an amount can vary in a broad range and is dependent on various factors, such as the plants to be controlled, the treated cultivated plant or material, the climatic conditions and the specific pyrimidine compound of formula (I) used.

The pyrimidine compounds of formula (I), their N-oxides, salts or derivatives can be converted into customary types of agrochemical compositions, e. g. solutions, emulsions, suspensions, dusts, powders, pastes, granules, pressings, capsules, and mixtures thereof. Examples for agrochemical composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further agrochemical compositions types are defined in the “Catalogue of pesticide formulation types and international coding system”, Technical Monograph No. 2, 6th Ed. May 2008, CropLife International.

The agrochemical compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic, and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, non-ionic, and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylaryl-sulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids, or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters, or monoglycerides. Examples of sugar-based surfactants are sorbitans, ethoxylated sorbitans, sucrose, and glucose esters, or alkylpoly-glucosides. Examples of polymeric surfactants are home- or copolymers of vinylpyrrolidone, vinylalcohols, or vinylacetate.

Suitable cationic surfactants are quaternary surfactants, e.g. quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B-C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the pyrimidine compounds of formula (1) on the target. Examples are surfactants, mineral or vegetable oils, and other auxiliaries.

Further examples are listed by Knowles, Adjuvants, and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea, and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo-, and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for agrochemical composition types and their preparation are:

i) Water-Soluble Concentrates (SL, LS)

10-60 wt % of a pyrimidine compound of formula (I) according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible Concentrates (DC)

5-25 wt % of a pyrimidine compound of formula (I) according to the invention and 1-10 wt % dispersant (e. g. polyvinylpyrrolidone) are dissolved in organic solvent (e.g. cyclohexanone) ad 100 wt %. Dilution with water gives a dispersion.

iii) Emulsifiable Concentrates (EC)

15-70 wt % of a pyrimidine compound of formula (I) according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

iv) Emulsions (EW, EO, ES)

5-40 wt % of a pyrimidine compound of formula (I) according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a pyrimidine compound of formula (I) according to the invention are comminuted with addition of 2-10 wt % dispersants and wetting agents (e.g. sodium lignosulfonate and alcohol ethoxylate), 0,1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

vi) Water-Dispersible Granules and Water-Soluble Granules (WG, SG)

50-80 wt % of a pyrimidine compound of formula (I) according to the invention are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a pyrimidine compound of formula (I) according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a pyrimidine compound of formula (I) according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

iv) Microemulsion (ME)

5-20 wt % of a pyrimidine compound of formula (I) according to the invention are added to 5-30 wt % organic solvent blend (e.g. fatty acid dimethylamide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.

iv) Microcapsules (CS)

An oil phase comprising 5-50 wt % of a pyrimidine compound of formula (I) according to the invention, 0-40 wt % water insoluble organic solvent (e.g. aromatic hydrocarbon), 2-15 wt % acrylic monomers (e.g. methylmethacrylate, methacrylic acid, and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a pyrimidine compound of formula (I) according to the invention, 0-40 wt % water insoluble organic solvent (e.g. aromatic hydrocarbon), and an isocyanate monomer (e.g. diphenylmethene-4,4′-diisocyanate) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition.

ix) Dustable Powders (DP, DS)

1-10 wt % of a pyrimidine compound of formula (I) according to the invention are ground finely and mixed intimately with solid carrier (e.g. finely divided kaolin) ad 100 wt %.

x) Granules (GR, FG)

0.5-30 wt % of a pyrimidine compound of formula (I) according to the invention is ground finely and associated with solid carrier (e.g. silicate) ad 100 wt %. Granulation is achieved by extrusion, spray-drying or the fluidized bed.

xi) Ultra-Low Volume Liquids (UL)

1-50 wt % of a pyrimidine compound of formula (I) according to the invention are dissolved in organic solvent (e.g. aromatic hydrocarbon) ad 100 wt %.

The agrochemical compositions types i) to xi) may optionally comprise further auxiliaries, such as 0,1-1 wt % bactericides, 5-15 wt % anti-freezing agents, 0,1-1 wt % anti-foaming agents, and 0,1-1 wt % colorants.

The agrochemical compositions comprising generally comprise between 0.01 and 95%, preferably between 0.1 and 90%, and in particular between 0.5 and 75%, by weight of the pyrimidine compound of formula (I). The pyrimidine compounds of formula (I) are employed in a purity of from 90% to 100%, preferably from 95% to 100% (according to NMR spectrum).

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The agrochemical compositions in question give, after two-to-tenfold dilution, active substance concentrations of from 0.01 to 60% by weight, preferably from 0.1 to 40% by weight, in the ready-to-use preparations. Application can be carried out before or during sowing.

Methods for applying pyrimidine compounds of formula (I) and agrochemical compositions thereof, on to plant propagation material, especially seeds, include dressing, coating, pelleting, dusting, soaking and in-furrow application methods of the propagation material. Preferably, pyrimidine compounds of formula (I) and agrochemical compositions thereof, are applied on to the plant propagation material by a method such that germination is not induced, e. g. by seed dressing, pelleting, coating and dusting.

Various types of oils, wetters, adjuvants, fertilizer, or micronutrients, and further pesticides (e.g. herbicides, insecticides, fungicides, growth regulators, safeners) may be added to the pyrimidine compounds of formula (I) and the agrochemical compositions comprising them as pre-mix or, if appropriate not until immediately prior to use (tank mix). These agents can be admixed with the agrochemical compositions according to the invention in a weight ratio of 1:100 to 100:1, preferably 1:10 to 10:1.

The user applies the pyrimidine compound of formula (I) according to the invention and the agrochemical compositions comprising them usually from a pre-dosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, either individual components of the agrochemical composition according to the invention or partially premixed components, e. g. components comprising pyrimidine compounds of formula (I) may be mixed by the user in a spray tank and further auxiliaries and additives may be added, if appropriate.

In a further embodiment, individual components of the agrochemical composition according to the invention such as parts of a kit or parts of a binary or ternary mixture may be mixed by the user himself in a spray tank and further auxiliaries may be added, if appropriate.

In a further embodiment, either individual components of the agrochemical composition according to the invention or partially premixed components, e. g components comprising pyrimidine compounds of formula (I) can be applied jointly (e.g. after tank mix) or consecutively.

The pyrimidine compounds of formula (I), are suitable as herbicides. They are suitable as such or as an appropriately formulated composition (agrochemical composition).

The pyrimidine compounds of formula (I), or the agrochemical compositions comprising the pyrimidine compounds of formula (I), control vegetation on non-crop areas very efficiently, especially at high rates of application. They act against broad-leaved weeds and grass weeds in crops such as wheat, rice, maize, soya, and cotton without causing any significant damage to the crop plants. This effect is mainly observed at low rates of application.

The pyrimidine compounds of formula (I), or the agrochemical compositions comprising them, are applied to the plants mainly by spraying the leaves. Here, the application can be carried out using, e.g., water as carrier by customary spraying techniques using spray liquor amounts of from about 100 to 1000 I/ha (e.g. from 300 to 400 I/ha). The pyrimidine compounds of formula (1), or the agrochemical compositions comprising them, may also be applied by the low-volume or the ultra-low-volume method, or in the form of micro granules.

Application of the pyrimidine compounds of formula (I), or the agrochemical compositions comprising them, can be done before, during, and/or after, preferably during and/or after, the emergence of the undesirable plants.

The pyrimidine compounds of formula (I), or the agrochemical compositions comprising them, can be applied pre-, post-emergence or pre-plant, or together with the seed of a crop plant. It is also possible to apply the pyrimidine compounds of formula (I), or the agrochemical compositions comprising them, by applying seed, pretreated with the pyrimidine compounds of formula (I), or the agrochemical compositions comprising them, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the pyrimidine compounds of formula (I), or the agrochemical compositions comprising them, can be applied by treating seed. The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the pyrimidine compounds of formula (I), or the agrochemical compositions prepared therefrom. Here, the herbicidal compositions can be applied diluted or undiluted.

The term “seed” comprises seed of all types, such as, e.g., corns, seeds, fruits, tubers, seedlings and similar forms. Here, preferably, the term seed describes corns and seeds. The seed used can be seed of the useful plants mentioned above, but also the seed of transgenic plants or plants obtained by customary breeding methods.

When employed in plant protection, the amounts of active substances applied, i.e. the pyrimidine compounds of formula (I) without formulation auxiliaries, are, depending on the kind of effect desired, from 0.001 to 2 kg per ha, preferably from 0.005 to 2 kg per ha, more preferably from 0.05 to 0.9 kg per ha and in particular from 0.1 to 0.75 kg per ha.

In another embodiment of the invention, the application rate of the pyrimidine compounds of formula (I) is from 0.001 to 3 kg/ha, preferably from 0.005 to 2.5 kg/ha and in particular from 0.01 to 2 kg/ha of active substance (a.s.).

In another preferred embodiment of the invention, the rates of application of the pyrimidine compounds of formula (I) according to the present invention (total amount of pyrimidine compounds of formula (I)) are from 0.1 g/ha to 3000 g/ha, preferably 10 g/ha to 1000 g/ha, depending on the control target, the season, the target plants and the growth stage.

In another preferred embodiment of the invention, the application rates of the pyrimidine compounds of formula (I) are in the range from 0.1 g/ha to 5000 g/ha and preferably in the range from 1 g/ha to 2500 g/ha or from 5 g/ha to 2000 g/ha.

In another preferred embodiment of the invention, the application rate of the pyrimidine compounds of formula (I) is 0.1 to 1000 g/ha, preferably 1 to 750 g/ha, more preferably 5 to 500 g/ha.

In treatment of plant propagation materials such as seeds, e. g. by dusting, coating or drenching seed, amounts of active substance of from 0.1 to 1000 g, preferably from 1 to 1000 g, more preferably from 1 to 100 g, and most preferably from 5 to 100 g, per 100 kilogram of plant propagation material (preferably seeds) are generally required.

In another embodiment of the invention, to treat the seed, the amounts of active substances applied, i.e. the pyrimidine compounds of formula (I) are generally employed in amounts of from 0.001 to 10 kg per 100 kg of seed.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Depending on the application method in question, the pyrimidine compounds of formula (I), or the agrochemical compositions comprising them, can additionally be employed in a further number of crop plants for eliminating undesirable plants. Examples of suitable crops are the following:

Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera, and Zea mays.

Preferred crops are Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N.rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (s. vulgare), Triticale, Triticum aestivum, Triticum durum, Vicia faba, Vitis vinifera, and Zea mays.

Especially preferred crops are crops of cereals, corn, soybeans, rice, oilseed rape, cotton, potatoes, peanuts, or permanent crops.

The pyrimidine compounds of formula (I) according to the invention, or the agrochemical compositions comprising them, can also be used in genetically modified plants. The term “genetically modified plants” is to be understood as plants whose genetic material has been modified by the use of recombinant DNA techniques to include an inserted sequence of DNA that is not native to that plant species' genome or to exhibit a deletion of DNA that was native to that species' genome, wherein the modification(s) cannot readily be obtained by cross breeding, mutagenesis or natural recombination alone. Often, a particular genetically modified plant will be one that has obtained its genetic modification(s) by inheritance through a natural breeding or propagation process from an ancestral plant whose genome was the one directly treated by use of a recombinant DNA technique. Typically, one or more genes have been integrated into the genetic material of a genetically modified plant in order to improve certain properties of the plant.

Such genetic modifications also include but are not limited to targeted post-translational modification of protein(s), oligo- or polypeptides. e. g., by inclusion therein of amino acid mutation(s) that permit, decrease, or promote glycosylation or polymer additions such as prenylation, acetylation farnesylation, or PEG moiety attachment.

Plants that have been modified by breeding, mutagenesis or genetic engineering, e.g. have been rendered tolerant to applications of specific classes of herbicides, such as auxin herbicides such as dicamba or 2,4-D; bleacher herbicides such as hydroxyphenylpyruvate dioxygenase (HPPD) inhibitors or phytoene desaturase (PDS) inhibitors; acetolactate synthase (ALS) inhibitors such as sulfonyl ureas or imidazolinones; enolpyruvyl shikimate 3-phosphate synthase (EPSP) inhibitors such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering; furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are, e.g., described in Pest Management Science 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein. Several cultivated plants have been rendered tolerant to herbicides by mutagenesis and conventional methods of breeding, e. g., Clearfield® summer rape (Canola, BASF SE, Germany) being tolerant to imidazolinones, e. g., imazamox, or ExpressSun® sunflowers (DuPont, USA) being tolerant to sulfonyl ureas, e. g., tribenuron. Genetic engineering methods have been used to render cultivated plants such as soybean, cotton, corn, beets and rape, tolerant to herbicides such as glyphosate, imidazolinones, and glufosinate, some of which are under development or commercially available under the brands or trade names RoundupReady® (glyphosate tolerant, Monsanto, USA), Cultivance® (imidazolinone tolerant, BASF SE, Germany), and LibertyLink® (glufosinate tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as delta-endotoxins, e. g., CryIA(b), CryIA(c), CryIF, CryIF(a2), CryIIA(b), CryIIIA, CryIIIB(b1) or Cry9c; vegetative insecticidal proteins (VIP), e. g., VIP1, VIP2, VIP3, or VIP3A; insecticidal proteins of bacteria colonizing nematodes, e. g., Photorhabdus spp. or Xenorhabdus spp.; toxins produced by animals, such as scorpion toxins, arachnid toxins, wasp toxins, or other insect-specific neurotoxins; toxins produced by fungi, such as Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as including pre-toxins, hybrid proteins, truncated or otherwise modified proteins. Hybrid proteins are characterized by a new combination of protein domains, (see, e. g., WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810, and WO 03/52073. The methods for producing such genetically modified plants are generally known to the person skilled in the art and are described, e. g., in the publications mentioned above.

These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g., Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g., EP-A 392 225), plant disease resistance genes (e. g., potato culti-vars, which express resistance genes acting against Phytophthora infestans derived from the Mexican wild potato, Solanum bulbocastanum) or T4-lyso-zym (e.g., potato cultivars capable of synthesizing these proteins with increased resistance against bacteria such as Erwinia amylovora). The methods for producing such genetically modified plants are generally known to the person skilled in the art and are described, e.g., in the publications mentioned above.

Furthermore, plants are also covered that are by the use of recombinant DNA techniques capable to synthesize one or more proteins to increase the productivity (e.g., bio-mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of ingredients or new ingredients, specifically to improve human or animal nutrition, e. g., oil crops that produce health-promoting long-chain omega-3 fatty acids or unsaturated omega-9 fatty acids (e. g., Nexera® rape, Dow AgroSciences, Canada).

Furthermore, plants are also covered that contain by the use of recombinant DNA techniques a modified amount of ingredients or new ingredients, specifically to improve raw material production, e.g., potatoes that produce increased amounts of amylopectin (e.g. Amflora® potato, BASF SE, Germany).

The preparation of the pyrimidine compounds of formula (I) is illustrated by the following examples.

### A Preparation Examples

### Example 1: [5-(3-Chloro-2-pyridyl)-2-cyclopropyl-pyrimidin-4-yl]methanol 1.1 5-Bromo-2-cyclopropyl-pyrimidine-4-carboxylic acid

NaH (60% purity, 41.9 g) is added in portions to EtOH (800 mL) at 0° C. The resulting mixture is warmed to ambient temperature and the cyclopropanecarboxamidine hydrochlorid (93.5 g) is added in portions. The reaction is warmed to 50° C. and maintained at this temperature for 0.5 h and then cooled to ambient temperature before mucobromic acid (100 g) is added in EtOH while keeping the internal temperature below 55° C. The mixture is allowed to cool to ambient temperature and stirred for additional 16 h. All solid components are removed by filtration and the resulting solution is concentrated under reduced pressure. Aq. HCl (1 mol/L) is added, the aqueous phase is washed with EtOAc (3×), the combined organic extracts are dried over MgSO₄ and the solid parts removed by filtration. The residue is concentrated under reduced pressure and the resulting solid titrated with (ⁱPr)₂O. The solids are collected by filtration and dried yielding the title compound (68.0 g, yield 72%) as a colorless solid.

¹H NMR (400 MHz, CDCl₃): δ=8.91 (s, 1H), 2.38-2.26 (m, 1H), 1.34-1.14 (m, 4H) ppm; MS (ESI) m/z 244.9 [M+H⁺].

### 1.2 Methyl 5-bromo-2-cyclopropyl-pyrimidine-4-carboxylate

5-Bromo-2-cyclopropyl-pyrimidine-4-carboxylic acid (91.0 g) is dissolved in MeOH (1200 mL) and conc. sulfuric acid (20 mL) is added dropwise at ambient temperature. The reaction is heated to reflux and stirred for 16 h. The resulting mixture is cooled to ambient temperature and neutralized with aq. sat. NaHCO₃. The residue is parted between EtOAc and aq. sat. NaHCO₃, the phases are separated, the aq. phase is extracted with EtOAc and the combined organics are dried over MgSO₄. Solid parts are removed by filtration and the organic phase is concentrated under reduced pressure. Column chromatography of the resulting crude product (ISCO-CombiFlash Rf, cyclohexane/EtOAc) yields the title compound (81.0 g, yield 84%) as a colorless solid.

¹H NMR (400 MHz, CDCl₃): δ=8.74 (s, 1H), 4.01 (s, 3H), 2.33-2.22 (m, 1H), 1.21-1.06 (m, 4H) ppm; MS (ESI) m/z 246.9 [M+H⁺].

### 1.3 Methyl 2-cyclopropyl-5-(4,4,5,5-tetramethyl-1,3,2-dioxaborolan-2-yl)pyrimidine-4-carboxylate

A mixture of methyl 2-cyclopropyl-5-(4,4,5,5-tetramethyl-1,3,2-dioxaborolan-2-yl)pyrimidine-4-carboxylate (1.00 g), 2,3-dibromopyridine (1.56 g), CsF (1.25 g) and PdCl2dppf (403 mg) in toluene (20 mL) is heated under reflux under an atmosphere of argon for 3 h. The volatiles are removed under reduced pressure and column chromatography of the crude product (cyclohexane/EtOAc) yields the title compound (300 mg, yield 27%) as a colorless solid.

¹H NMR (400 MHz, CDCl₃): δ 8.78 (s, 1H), 8.62 (dd, J=4.7, 1.5 Hz, 1H), 7.99 (dd, J=8.1, 1.5 Hz, 1H), 7.23 (dd, J=8.1, 4.7 Hz, 1H), 3.82 (s, 3H), 2.47-2.41 (m, 1H), 1.39-1.25 (m, 2H), 1.20-1.15 (m, 2H) ppm; MS (ESI) m/z 336.0 [M+H⁺].

### 1.4 Methyl 5-(3-chloro-2-pyridyl)-2-cyclopropyl-pyrimidine-4-carboxylate

A mixture of methyl 2-cyclopropyl-5-(4,4,5,5-tetramethyl-1,3,2-dioxaborolan-2-yl)pyrimidine-4-carboxylate (33.0 g), 2-Bromo-3-chloropyridine (22.7 g), CsF (41.2 g) and PdCl₂dppf (13.3 g) in toluene (350 mL) is heated under reflux under an atmosphere of argon for 3 h. The volatiles are removed under reduced pressure and column chromatography of the crude product (cyclohexane/EtOAc) yields the title compound (10.0 g, yield 32%) as a colorless solid.

¹H NMR (400 MHz, CDCl₃): δ 8.81 (s, 1H), 8.58 (dd, J=4.8, 1.1 Hz, 1H), 7.81 (dd, J=8.1, 1.3 Hz, 1H), 7.31 (dd, J=8.2, 4.7 Hz, 1H), 3.82 (s, 3H), 2.47-2.40 (m, 1H), 1.29-1.23 (m, 2H), 1.20-1.15 (m, 2H) ppm; MS (ESI) m/z 290.0 [M+H⁺].

### 1.5 5-(3-Chloro-2-pyridyl)-2-cyclopropyl-pyrimidine-4-carboxylic acid

Methyl 5-(3-chloro-2-pyridyl)-2-cyclopropyl-pyrimidine-4-carboxylate (1.30 g) is dissolved in a mixture THE/water (vol/vol=1/1; 50 mL) and lithiumhydroxid (215 mg) is added as a solution in water. The resulting mixture is stirred for 20 hours at ambient temperature and then acidified to pH=2-3 with aqueous hydrochloric acid (2 mol/L). The aq. phase is extracted with EtOAc (3×) and the combined organics are dried over Na₂SO₄. Solid parts are removed by filtration and the organic phase is concentrated under reduced pressure and the residues are triturated with CH₂Cl₂ yielding the title compound (850 mg, 69%) as a colorless solid.

¹H NMR (400 MHz, CDCl₃): δ 8.79 (s, 1H), 8.59 (dd, J=4.8, 1.4 Hz, 1H), 7.83 (dd, J=8.1, 1.3 Hz, 1H), 7.36 (dd, J=8.2, 4.7 Hz, 1H), 2.46-2.40 (m, 1H), 1.32-1.23 (m, 4H) ppm; MS (ESI) m/z 275.9 [M+H⁺].

### 1.6 [5-(3-Chloro-2-pyridyl)-2-cyclopropyl-pyrimidin-4-yl]methanol

To a solution of 5-(3-Chloro-2-pyridyl)-2-cyclopropyl-pyrimidine-4-carboxylic acid (15.0 g) in THE (20 mL) CDI (10.0 g) is added in small portions. The resulting mixture is stirred for 1 h at ambient temperature and cooled to 0° C. before NaBH₄ (4.11 g) in ice-cold water (10 mL) is added. After stirring for two additional hours at ambient temperature, EtOAc is added and the phases are separated. The aq. phase is extracted with EtOAc (2×), the combined organics are washed with water and dried over Na₂SO₄. Solid parts are removed by filtration, the remaining solution is concentrated under reduced pressure and column chromatography of the crude product (cyclohexane/EtOAc) yields the title compound (5.00 g, yield 33%).

¹H NMR (400 MHz, CDCl₃): δ 8.61 (s, 1H), 8.60 (dd, J=4.7, 1.5 Hz, 1H), 7.87 (dd, J=8.2, 1.5 Hz, 1H), 7.34 (dd, J=8.2, 4.7 Hz, 1H), 4.56 (s, 2H), 2.40-2.34 (m, 1H), 1.28-1.24 (m, 2H), 1.19-1.14 (m, 2H) ppm; MS (ESI) m/z 262.0 [M+H⁺].

### Example 2: 5-(3-Bromo-2-pyridyl)-2-cyclopropyl-4-(3-furyl)pyrimidine

2.1 2-(3-Bromo-2-pyridyl)-1-(3-furyl)ethanone:LHMDS (1.06 mol/L in THF, 300 mL) is added to a solution of 3-bromo-2-methyl-pyridine (50.0 g) in dry THF (500 mL) maintained at 0° C., followed by the dropwise addition of ethyl furan-3-carboxylate (45.0 g) in THF. The reaction mixture is warmed to ambient temperature and stirred for 16 h. Citric acid (15% m/m) is added and the mixture is extracted with EtOAc (3×). The combined organics are dried over Na₂SO₄, solid parts are removed by filtration and the remaining solution is concentrated under reduced pressure. The resulting crude product (87.0 g) is used directly in the next step. MS (ESI) m/z 265.9 [M+H⁺].

2.2 2-(3-Bromo-2-pyridyl)-3-(dimethylamino)-1-(3-furyl)prop-2-en-1-one: The resulting crude product (87.0 g, see above) containing 2-(3-bromo-2-pyridyl)-1-(3-furyl)ethanone is stirred in N,N-DMF dimethyl acetal (550 mL) for 3 h at 75° C. Volatiles are removed under reduced pressure.

Purification by column chromatography (gradient from EtOAc to EtOAc/MeOH=4/1) yields the desired product (29.0 g, 28%). MS (ESI) m/z 322.9 [M+H⁺].

### 2.3 5-(3-Bromo-2-pyridyl)-2-cyclopropyl-4-(3-furyl)pyrimidine

To a solution of 2-(3-bromo-2-pyridyl)-3-(dimethylamino)-1-(3-furyl)prop-2-en-1-one (29.0 g) in EtOH (500 mL) was added cyclopropanecarboxamidine hydrochloride (10.9 g) and NaOEt (20%, 30.7 g). The mixture is refluxed for 4 h, cooled to ambient temperature and filtered. Volatiles are removed under reduced pressure and the crude product is purified by column chromatography (cyclohexane/EtOAc) to yield the title compound (21.2 g, 69%) as a yellow oil.

¹H NMR (400 MHz, CDCl₃): δ 8.70 (dd, J=4.7, 1.5 Hz, 1H), 8.46 (s, 1H), 8.01 (dd, J=8.1, 1.5 Hz, 1H), 7.31-7.28 (m, 2H), 7.23 (dd, J=1.6, 0.8 Hz, 1H), 6.37 (dd, J=1.9, 0.8 Hz, 1H), 2.38-2.31 (m, 1H), 1.27-1.23 (m, 2H), 1.14-1.10 (m, 2H) ppm; MS (ESI) m/z 343.9 [M+H⁺].

With appropriate modification of the starting materials, the procedures given in the synthesis examples below were used to obtain further compounds of formula I. The compounds obtained in this manner are listed in the table that follows, together with physical data.

The products shown below were characterized by melting point determination, by the masses ([m/z]) or retention time (RT; [min.]) determined by HPLC-MS or HPLC spectrometry.

HPLC-MS=high performance liquid chromatography-coupled mass spectrometry; HPLC methods:

Method: Column: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.25 min; flow: 0.8-1.0 ml/min in 1.51 minutes at 60° C.MS: quadrupole electrospray ionization, 80 V (positive mode).

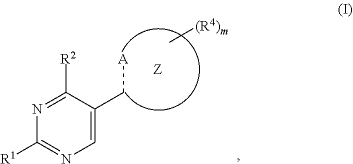

TABLE 1
# denotes point of attachment to the pyrimidine ring.

| Ex. | R¹ | R² | 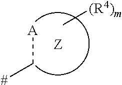 | HPLC/ MS, Rₜ [min] | HPLC/ MS, m/z |
|---|---|---|---|---|---|
| Ex.1 | c-C₃H₅ | CH₃ | 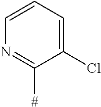 | 0.952 | 246.0 |
| Ex.2 | c-C₃H₅ | CH₃ | 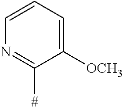 | 0.738 | 241.0 |
| Ex.3 | c-C₃H₅ | CH₃ | 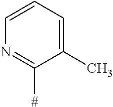 | 0.686 | 226.0 |
| Ex.4 | c-C₃H₅ | CH₃ | 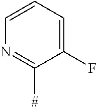 | 0.922 | 230.0 |
| Ex.5 | c-C₃H₅ | CH₃ | 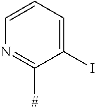 | 0.990 | 338.2 |
| Ex.6 | c-C₃H₅ | 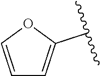 [SMILES: *c1ccco1] | 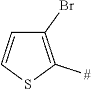 | 1.277 | 349.0 |
| Ex.7 | c-C₃H₅ | CH₃ | 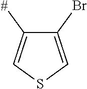 | 1.132 | 296.9 |
| Ex.8 | c-C₃H₅ | 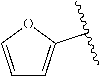 [SMILES: *c1ccco1] | 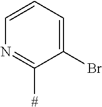 | 1.108 | 343.9 |
| Ex.9 | c-C₃H₅ | 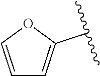 [SMILES: *c1ccco1] | 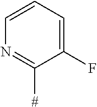 | 1.027 | 282.0 |
| Ex.10 | c-C₃H₅ | 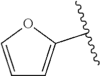 [SMILES: *c1ccco1] | 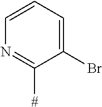 | 1.134 | 342.0 |
| Ex.11 | c-C₃H₅ | 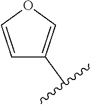 [SMILES: *c1ccoc1] | 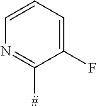 | 1.074 | 282.1 |
| Ex.12 | c-C₃H₅ | 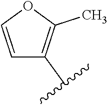 [SMILES: *c1ccoc1C] | 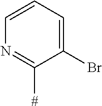 | 1.146 | 358.0 |
| Ex.13 | c-C₃H₅ | 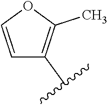 [SMILES: *c1ccoc1C] | 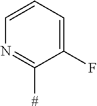 | 1.128 | 296.1 |
| Ex.14 | c-C₃H₅ | 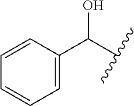 [SMILES: *C(O)c1ccccc1] | 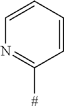 | 0.891 | 304.1 |
| Ex.15 | c-C₃H₅ | CH₃ | 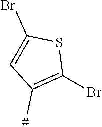 | 1.293 | 374.8 |
| Ex.16 | c-C₃H₅ | 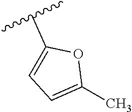 [SMILES: *c1ccc(C)o1] | 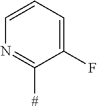 | 1.105 | 296.0 |
| Ex.17 | c-C₃H₅ | 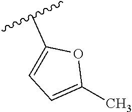 [SMILES: *c1ccc(C)o1] | 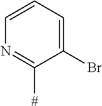 | 1.145 | 358.0 |
| Ex.18 | c-C₃H₅ | 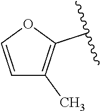 [SMILES: *c1occc1C] | 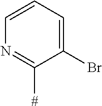 | 1.142 | 355.9 |
| Ex.19 | c-C₃H₅ | CH₂OH | 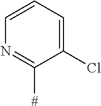 | 0.895 | 262.0 |
| Ex.20 | c-C₃H₅ | 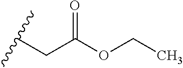 [SMILES: *CC(=O)OCC] | 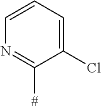 | 1.050 | 318.0 |
| Ex.21 | c-C₃H₅ | 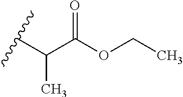 [SMILES: *C(C)C(=O)OCC] | 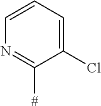 | 1.142 | 332.0 |
| Ex.22 | c-C₃H₅ | 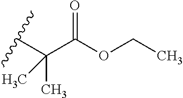 [SMILES: *C(C)(C)C(=O)OCC] | 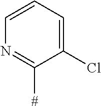 | 1.236 | 346.0 |
| Ex.23 | c-C₃H₅ | 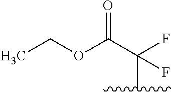 [SMILES: *C(F)(F)C(=O)OCC] | 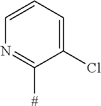 | 1.405 | 383.0 |
| Ex.24 | c-C₃H₅ | 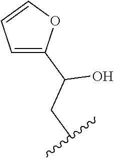 [SMILES: *CC(O)c1ccco1] | 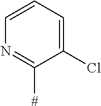 | 1.011 | 342.1 |
| Ex.25 | c-C₃H₅ | 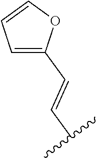 [SMILES: */C=C/c1ccco1] | 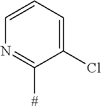 | 1.178 | 324.0 |
| Ex.26 | c-C₃H₅ | 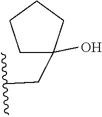 [SMILES: *CC1(O)CCCC1] | 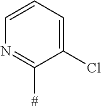 | 1.109 | 330.1 |
| Ex.27 | c-C₃H₅ | 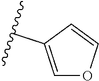 [SMILES: *c1ccoc1] | 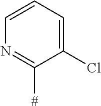 | 1.098 | 298.0 |
| Ex.28 | c-C₃H₅ | 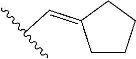 [SMILES: *C=C1CCCC1] | 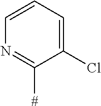 | 1.180 | 312.0 |
| Ex.29 | c-C₃H₅ | 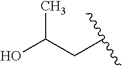 [SMILES: *CC(C)O] | 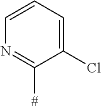 | 0.914 | 290.1 |
| Ex.30 | c-C₃H₅ | 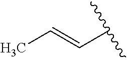 [SMILES: */C=C/C] | 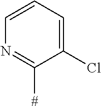 | 1.070 | 272.1 |
| Ex.31 | c-C₃H₅ | 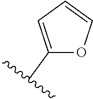 [SMILES: *c1ccco1] | 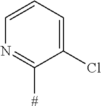 | 1.068 | 298.0 |
| Ex.32 | c-C₃H₅ | 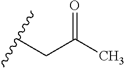 [SMILES: *CC(C)=O] | 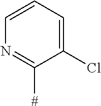 | 0.823 | 286.0 |
| Ex.33 | c-C₃H₅ | 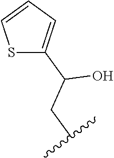 [SMILES: *CC(O)c1cccs1] | 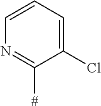 | 1.064 | 358.0 |
| Ex.34 | c-C₃H₅ | 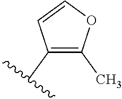 [SMILES: *c1ccoc1C] | 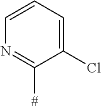 | 1.116 | 312.0 |
| Ex.35 | c-C₃H₅ | 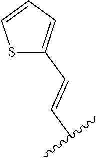 [SMILES: */C=C/c1cccs1] | 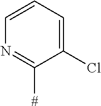 | 1.258 | 339.9 |
| Ex.36 | c-C₃H₅ | 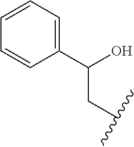 [SMILES: *CC(O)c1ccccc1] | 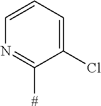 | 1.115 | 351.9 |
| Ex.37 | c-C₃H₅ | 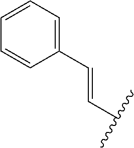 [SMILES: */C=C/c1ccccc1] | 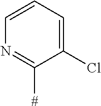 | 1.272 | 334.1 |
| Ex.38 | c-C₃H₅ | 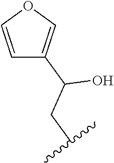 [SMILES: *CC(O)c1ccoc1] | 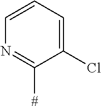 | 0.985 | 342.1 |
| Ex.39 | c-C₃H₅ | 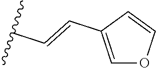 [SMILES: */C=C/c1ccoc1] | 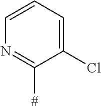 | 1.140 | 324.0 |
| Ex.40 | c-C₃H₅ | 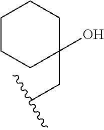 [SMILES: *CC1(O)CCCCC1] | 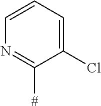 | 1.187 | 344.1 |
| Ex.41 | c-C₃H₅ | 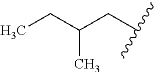 [SMILES: *CC(C)CC] | 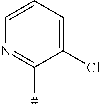 | 1.011 | 303.9 |
| Ex.42 | c-C₃H₅ | 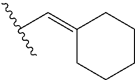 [SMILES: *C=C1CCCCC1] | 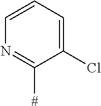 | 1.259 | 326.1 |
| Ex.43 | c-C₃H₅ | 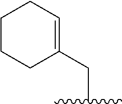 [SMILES: *CC1=CCCCC1] | 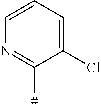 | 1.304 | 326.1 |
| Ex.44 | c-C₃H₅ | 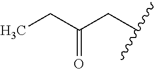 [SMILES: *CC(=O)CC] | 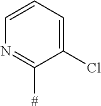 | 0.923 | 300.1 |
| Ex.45 | c-C₃H₅ | 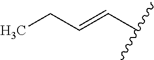 [SMILES: */C=C/CC] | 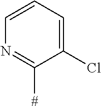 | 1.180 | 286.1 |
| Ex.46 | c-C₃H₅ | 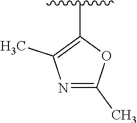 | 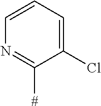 | 1.043 | 327.1 |
| Ex.47 | c-C₃H₅ | 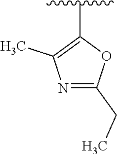 [SMILES: *c1oc(CC)nc1C] | 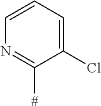 | 1.131 | 340.9 |

### B Use Examples

The herbicidal activity of the pyrimidine compounds of formula (I) was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles.

The containers were irrigated gently to promote germination and growth and subsequently covered with transparent plastic hoods until the plants had rooted. This cover caused uniform germination of the test plants, unless this had been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C., respectively.

The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments were of the following species:

| Bayer code | Scientific name |
|---|---|
| MATIN | Matricaria inodora |
| ALOMY | Alopecurus myosuroides |
| AMARE | Amaranthus retroflexus |
| APESV | Apera spica-venti |
| AVEFA | Avena fatua |
| ECHCG | Echinocloa crus-galli |
| SETVI | Setaria viridis |

At an application rate of 1000 g/ha, Ex.6 applied by the post-emergence method showed very good herbicidal activity against SETVI.

At an application rate of 2000 g/ha, Ex.8 applied by the pre-emergence method showed very good herbicidal activity against APESV.

At an application rate of 2000 g/ha, Ex.9 applied by the post-emergence method showed good herbicidal activity against ECHCG.

At an application rate of 2000 g/ha, Ex.10 applied by the post-emergence method showed good herbicidal activity against ALOMY.

At an application rate of 1000 g/ha, Ex.11 applied by the post-emergence method showed good herbicidal activity against ECHCG.

At an application rate of 2000 g/ha, Ex.12 applied by the post-emergence method showed good herbicidal activity against AVEFA, ECHCG and ALOMY, and applied by the pre-emergence method showed very good herbicidal activity against APESV.

At an application rate of 2000 g/ha, Ex.13 applied by the post-emergence method showed good herbicidal activity against ECHCG and SETVI.

At an application rate of 2000 g/ha, Ex.17 applied by the post-emergence method showed very good herbicidal activity against AVEFA, and applied by the pre-emergence method showed very good herbicidal activity against APESV and ECHCG.

At an application rate of 2000 g/ha, Ex.18 applied by the post-emergence method showed very good herbicidal activity against AFEVA, good herbicidal activity against and ECHCG.

At an application rate of 2000 g/ha, Ex.01 applied by the post-emergence method showed very good herbicidal activity against MATIN and AMARE.

At an application rate of 2000 g/ha, Ex. 14 applied by the post-emergence method showed very good herbicidal activity against SETVI.

At an application rate of 500 g/ha, Ex.15 applied by the pre-emergence method showed good herbicidal activity against ECHCG.

At an application rate of 2000 g/ha, Ex.20 applied by the post-emergence method showed very good herbicidal activity against AVEFA, ECHCG and ALOMY, and applied by the pre-emergence method showed very good herbicidal activity against ECHCG.

At an application rate of 2000 g/ha, Ex.21 applied by the post-emergence method showed very good herbicidal activity against ECHCG, and good herbicidal activity against AMARE, and applied by the pre-emergence method showed good herbicidal activity against APESV.

At an application rate of 2000 g/ha, Ex.22 applied by the post-emergence method showed very good herbicidal activity against AMARE.

At an application rate of 2000 g/ha, Ex.23 applied by the post-emergence method showed very good herbicidal activity against SETVI, ALOMY and AMARE.

At an application rate of 2000 g/ha, Ex.24 applied by the post-emergence method showed very good herbicidal activity against ALOMY, and good herbicidal activity against AVEFA and SETVI, and applied by the pre-emergence method showed very good herbicidal activity against APESV and ECHCG.

At an application rate of 2000 g/ha, Ex.25 applied by the post-emergence method showed very good herbicidal activity against ALOMY, SETVI and ECHCG, and applied by the pre-emergence method showed very good herbicidal activity against APESV and ECHCG.

At an application rate of 2000 g/ha, Ex.26 applied by the post-emergence method showed very good herbicidal activity against SETVI.

At an application rate of 2000 g/ha, Ex.27 applied by the post-emergence method showed very good herbicidal activity against ALOMY, AVEFA and ECHCG, and applied by the pre-emergence method showed very good herbicidal activity against APESV and ECHCG.

At an application rate of 2000 g/ha, Ex.28 applied by the post-emergence method showed very good herbicidal activity against ALOMY and ECHCG, and good herbicidal activity against AVEFA, and applied by the pre-emergence method showed very good herbicidal activity against APESV and ECHCG.

At an application rate of 2000 g/ha, Ex.29 applied by the post-emergence method showed very good herbicidal activity against SETVI, and good herbicidal activity against ABUTH, and applied by the pre-emergence method showed very good herbicidal activity against APESV and ECHCG.

At an application rate of 2000 g/ha, Ex.30 applied by the post-emergence method showed very good herbicidal activity against ALOMY, and good herbicidal activity against ECHCG, and applied by the pre-emergence method showed very good herbicidal activity against APESV and ECHCG.

At an application rate of 2000 g/ha, Ex.31 applied by the post-emergence method showed very good herbicidal activity against ALOMY, SETVI and ECHCG, and applied by the pre-emergence method showed very good herbicidal activity against APESV and ECHCG.

At an application rate of 2000 g/ha, Ex.32 applied by the post-emergence method showed very good herbicidal activity against ALOMY and AMARE, and good herbicidal activity against AVEFA, and applied by the pre-emergence method showed very good herbicidal activity against APESV.

At an application rate of 2000 g/ha, Ex.33 applied by the post-emergence method showed very good herbicidal activity against AMARE, and good herbicidal activity against ECHCG.

At an application rate of 2000 g/ha, Ex.34 applied by the post-emergence method showed very good herbicidal activity against ALOMY and AMARE, and good herbicidal activity against AVEFA, and applied by the pre-emergence method showed very good herbicidal activity against APESV and ECHCG.

At an application rate of 2000 g/ha, Ex.35 applied by the post-emergence method showed very good herbicidal activity against ALOMY and SETVI, and good herbicidal activity against AVEFA, and applied by the pre-emergence method showed very good herbicidal activity against APESV and ECHCG.

At an application rate of 2000 g/ha, Ex.36 applied by the post-emergence method showed very good herbicidal activity against ALOMY, ECHCG and SETVI.

At an application rate of 2000 g/ha, Ex.37 applied by the post-emergence method showed very good herbicidal activity against ABUTH, AMARE and SETVI.

At an application rate of 2000 g/ha, Ex.38 applied by the post-emergence method showed very good herbicidal activity against ECHCG, and applied by the pre-emergence method showed very good herbicidal activity against and ECHCG.

At an application rate of 2000 g/ha, Ex.39 applied by the post-emergence method showed good herbicidal activity against ALOMY, and very good herbicidal activity against ECHCG, and applied by the pre-emergence method showed very good herbicidal activity against APESV and ECHCG.

At an application rate of 2000 g/ha, Ex.41 applied by the post-emergence method showed good herbicidal activity against ECHCG, and applied by the pre-emergence method showed very good herbicidal activity against APESV and ECHCG.

At an application rate of 2000 g/ha, Ex.42 applied by the post-emergence method showed good herbicidal activity against ECHCG.

At an application rate of 2000 g/ha, Ex.44 applied by the post-emergence method showed very good herbicidal activity against ALOMY and good herbicidal activity against AMARE, and applied by the pre-emergence method showed very good herbicidal activity against APESV and ECHCG.

At an application rate of 2000 g/ha, Ex.45 applied by the post-emergence method showed good herbicidal activity against ALOMY, and applied by the pre-emergence method showed very good herbicidal activity against ECHCG and good herbicidal activity against APESV.

At an application rate of 2000 g/ha, Ex.46 applied by the post-emergence method showed very good herbicidal activity against AVEFA, SETVI and ECHCG, and applied by the pre-emergence method showed very good herbicidal activity against APESV and ECHCG.

1-15. (canceled)

16. A pyrimidine compound of formula (I) 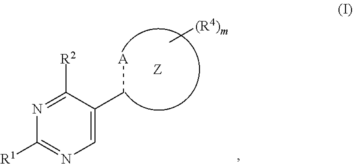
wherein
the dotted line ( [symbol] ) is a single bond or a double bond;
R¹ is C₁-C₆-alkyl, C₁-C₆-haloalkyl, HO—C₁-C₆-alkyl, C₃-C₆-alkenyl, C₃-C₆-haloalkenyl, C₂-C₆-alkynyl, C₃-C₆-haloalkynyl, C₁-C₆-alkoxy-C₁-C₆-alkyl, C₁-C₆-alkoxy, C₃-C₆-alkenyloxy, C₃-C₆-haloalkenyloxy, C₃-C₆-alkynyloxy, C₃-C₆-haloalkynyloxy, C₁-C₆-haloalkoxy, C₃-C₆-cycloalkoxy, C₃-C₆-halocycloalkoxy, C₃-C₆-cycloalkenyloxy, C₃-C₆-halocycloalkenyloxy, C₁-C₆-alkylthio, C₁-C₆-haloalkylthio, (C₁-C₆-alkyl)amino, di(C₁-C₆-alkyl)amino, C₁-C₆-alkylsulfinyl, C₁-C₆-alkylsulfonyl, C₃-C₆-cycloalkyl, C₃-C₆-cycloalkenyl, C₃-C₆-halocycloalkyl, C₃-C₆-halocycloalkenyl, [1-(C₁-C₆-alkyl)]-C₃-C₆-cycloalkyl, [1-(C₃-C₆-alkenyl)]-C₃-C₆-cycloalkyl, [1-(C₂-C₆-alkynyl)]-C₃-C₆-cycloalkyl, [1-(C₁-C₆-haloalkyl)]-C₃-C₆-cycloalkyl, [1-(C₃-C₆-haloalkenyl)]-C₃-C₆-cycloalkyl, [1-(C₃-C₆-haloalkynyl)]-C₃-C₆-cycloalkyl, C₃-C₆-cycloalkyl-C₁-C₆-alkyl, C₃-C₆-cycloalkyl-C₁-C₆-haloalkyl, C₃-C₆-cycloalkyl-C₁-C₆-alkoxy, C₃-C₆-cycloalkyl-C₁-C₆-haloalkoxy, phenyl, 5- or 6-membered heteroaryl, or 3- to 6-membered heterocyclyl;
wherein the cyclic groups of R¹ are unsubstituted or substituted by R^{a};
R² is C₁-C₆-alkyl, C₁-C₆-haloalkyl, C₂-C₆-alkenyl, C₂-C₆-haloalkenyl, C₁-C₆-alkoxy-C₂-C₆-alkenyl, C₁-C₆-alkoxy-C₂-C₆-haloalkenyl, C₁-C₆-haloalkoxy-C₂-C₆-alkenyl, C₁-C₆-haloalkoxy-C₂-C₆-haloalkenyl, C₂-C₆-alkynyl, C₂-C₆-haloalkynyl, C₁-C₆-alkoxy-C₂-C₆-alkynyl, C₁-C₆-alkoxy-C₃-C₆-haloalkynyl, C₁-C₆-haloalkoxy-C₂-C₆-alkynyl, C₁-C₆-haloalkoxy-C₃-C₆-haloalkynyl, C₃-C₆-cycloalkyl, C₃-C₆-halocycloalkyl, C₃-C₆-cycloalkenyl, C₃-C₆-halocycloalkenyl, C₃-C₆-cycloalkyl-C₁-C₆-alkyl, C₃-C₆-cycloalkyl-C₁-C₆-haloalkyl, C₃-C₆-halocycloalkyl-C₁-C₆-alkyl, C₃-C₆-halocycloalkyl-C₁-C₆-haloalkyl, C₃-C₆-cycloalkenyl-C₁-C₆-alkyl, C₃-C₆-cycloalkenyl-C₁-C₆-haloalkyl, C₃-C₆-halocycloalkenyl-C₁-C₆-alkyl, C₃-C₆-halocycloalkenyl-C₁-C₆-haloalkyl, C₃-C₆-cycloalkyl-C₂-C₆-alkenyl, C₃-C₆-cycloalkyl-C₂-C₆-haloalkenyl, C₃-C₆-halocycloalkyl-C₂-C₆-alkenyl, C₃-C₆-halocycloalkyl-C₂-C₆-haloalkenyl, C₃-C₆-cycloalkenyl-C₂-C₆-alkenyl, C₃-C₆-cycloalkenyl-C₂-C₆-haloalkenyl, C₃-C₆-halocycloalkenyl-C₂-C₆-alkenyl, C₃-C₆-halocycloalkenyl-C₂-C₆-haloalkenyl, C₃-C₆-cycloalkyl-C₂-C₆-alkynyl, C₃-C₆-cycloalkyl-C₃-C₆-haloalkynyl, C₃-C₆-halocycloalkyl-C₂-C₆-alkynyl, C₃-C₆-halocycloalkyl-C₃-C₆-haloalkynyl, C₃-C₆-cycloalkenyl-C₂-C₆-alkynyl, C₃-C₆-cycloalkenyl-C₃-C₆-haloalkynyl, C₃-C₆-halocycloalkenyl-C₂-C₆-alkynyl, C₃-C₆-halocycloalkenyl-C₃-C₆-haloalkynyl, C₃-C₆-cycloalkyl-C₁-C₆-alkylidenyl, C₃-C₆-cycloalkyl-C₂-C₆-haloalkylidenyl, C₃-C₆-halocycloalkyl-C₁-C₆-alkylidenyl, C₃-C₆-halocycloalkyl-C₂-C₆-haloalkylidenyl, C₃-C₆-cycloalkenyl-C₁-C₆-alkylidenyl, C₃-C₆-cycloalkenyl-C₂-C₆-haloalkylidenyl, C₃-C₆-halocycloalkenyl-C₁-C₆-alkylidenyl, C₃-C₆-halocycloalkenyl-C₂-C₆-haloalkylidenyl, heterocyclyl-C₁-C₆-alkylidenyl, heterocyclyl-C₂-C₆-haloalkylidenyl, C₃-C₆-hydroxycycloalkyl-C₁-C₆-alkyl, C₃-C₆-hydroxycycloalkyl-C₁-C₆-haloalkyl, C₃-C₆-hydroxycycloalkenyl-C₁-C₆-alkyl, C₃-C₆-hydroxycycloalkenyl-C₁-C₆-haloalkyl, C₁-C₆-hydroxyalkyl, C₂-C₆-hydroxyhaloalkyl, C₃-C₆-hydroxyalkenyl, C₃-C₆-hydroxyhaloalkenyl, C₃-C₆-hydroxyalkynyl, C₄-C₆-hydroxyhaloalkynyl, C₃-C₆-hydroxycycloalkyl, C₃-C₆-hydroxyhalocycloalkyl, C₃-C₆-hydroxycycloalkenyl, C₃-C₆-hydroxyhalocycloalkenyl, C₃-C₆-cycloalkyl-C₁-C₆-hydroxyalkyl, C₃-C₆-cycloalkyl-C₂-C₆-hydroxyhaloalkyl, C₃-C₆-halocycloalkyl-C₁-C₆-hydroxyalkyl, C₃-C₆-halocycloalkyl-C₂-C₆-hydroxyhaloalkyl, C₃-C₆-cycloalkenyl-C₁-C₆-hydroxyalkyl, C₃-C₆-cycloalkenyl-C₂-C₆-hydroxyhaloalkyl, C₃-C₆-halocycloalkenyl-C₁-C₆-hydroxyalkyl, C₃-C₆-halocycloalkenyl-C₂-C₆-hydroxyhaloalkyl, C₃-C₆-cycloalkyl-C₃-C₆-hydroxyalkenyl, C₃-C₆-cycloalkyl-C₃-C₆-hydroxyhaloalkenyl, C₃-C₆-halocycloalkyl-C₃-C₆-hydroxyalkenyl, C₃-C₆-halocycloalkyl-C₃-C₆-hydroxyhaloalkenyl, C₃-C₆-cycloalkenyl-C₃-C₆-hydroxyalkenyl, C₃-C₆-cycloalkenyl-C₃-C₆-hydroxyhaloalkenyl, C₃-C₆-halocycloalkenyl-C₃-C₆-hydroxyalkenyl, C₃-C₆-halocycloalkenyl-C₃-C₆-hydroxyhaloalkenyl, C₃-C₆-cycloalkyl-C₃-C₆-hydroxyalkynyl, C₃-C₆-halocycloalkyl-C₃-C₆-hydroxyalkynyl, C₃-C₆-cycloalkenyl-C₃-C₆-hydroxyalkynyl, C₃-C₆-halocycloalkenyl-C₃-C₆-hydroxyalkynyl, C₃-C₆-cycloalkyl-C₂-C₆-hydroxyalkylidenyl, C₃-C₆-halocycloalkyl-C₂-C₆-hydroxyalkylidenyl, C₃-C₆-cycloalkenyl-C₂-C₆-hydroxyalkylidenyl, C₃-C₆-halocycloalkyl-C₂-C₆-hydroxyalkylidenyl, heterocyclyl-C₂-C₆-hydroxyalkylidenyl, hydroxycarbonyl-C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-hydroxyalkyl, C₁-C₆-haloalkoxycarbonyl-C₁-C₆-hydroxyalkyl, C₁-C₆-alkoxycarbonyl-C₁-C₆-haloalkyl, C₁-C₆-haloalkoxycarbonyl-C₁-C₆-haloalkyl, C₁-C₆-alkoxycarbonyl-C₁-C₆-alkyl, C₁-C₆-haloalkoxycarbonyl-C₁-C₆-alkyl, C₃-C₆-hydroxycycloalkyl-C₁-C₆-hydroxyalkyl, C₃-C₆-hydroxycycloalkenyl-C₁-C₆-hydroxyalkyl, C₃-C₆-hydroxycycloalkyl-C₃-C₆-hydroxyalkenyl, C₃-C₆-hydroxycycloalkenyl-C₃-C₆-hydroxyalkenyl, C₃-C₆-hydroxycycloalkyl-C₃-C₆-hydroxyalkynyl, C₃-C₆-hydroxycycloalkenyl-C₃-C₆-hydroxyalkenyl, C₂-C₆-dihydroxyalkyl, C₃-C₆-dihydroxyhaloalkyl, C₄-C₆-dihydroxyalkenyl, C₄-C₆-dihydroxyhaloalkenyl, C₄-C₆-dihydroxyalkynyl, C₅-C₆-dihydroxyhaloalkynyl, C₄-C₆-dihydroxycycloalkyl, C₄-C₆-dihydroxyhalocycloalkyl, C₄-C₆-dihydroxycycloalkenyl, C₄-C₆-dihydroxyhalocycloalkenyl, C₃-C₆-cycloalkyl-C₂-C₆-dihydroxyalkyl, C₃-C₆-halocycloalkyl-C₂-C₆-dihydroxyalkyl, C₃-C₆-cycloalkenyl-C₂-C₆-dihydroxyalkyl, C₃-C₆-halocycloalkenyl-C₂-C₆-dihydroxyalkyl, C₃-C₆-cycloalkyl-C₃-C₆-dihydroxyalkenyl, C₃-C₆-halocycloalkyl-C₃-C₆-dihydroxyalkenyl, C₃-C₆-cycloalkenyl-C₃-C₆-dihydroxyalkenyl, C₃-C₆-halocycloalkenyl-C₃-C₆-dihydroxyalkenyl, C₃-C₆-cycloalkyl-C₄-C₆-dihydroxyalkynyl, C₃-C₆-halocycloalkyl-C₄-C₆-dihydroxyalkynyl, C₃-C₆-cycloalkenyl-C₄-C₆-dihydroxyalkynyl, C₃-C₆-halocycloalkyl-C₄-C₆-dihydroxyalkynyl, C₃-C₆-cycloalkyl-C₃-C₆-dihydroxyalkylidenyl, C₃-C₆-halocycloalkyl-C₃-C₆-dihydroxyalkylidenyl, heterocyclyl-C₃-C₆-dihydroxyalkylidenyl, hydroxycarbonyl-C₂-C₆-dihydroxyalkyl, hydroxycarbonyl-C₃-C₆-dihydroxyhaloalkyl, C₁-C₆-alkoxycarbonyl-C₂-C₆-dihydroxyalkyl, C₁-C₆-haloalkoxycarbonyl-C₂-C₆-dihydroxyalkyl, C₁-C₆-haloalkoxycarbonyl-C₃-C₆-dihydroxyhaloalkyl, C₃-C₆-dihydroxycycloalkyl-C₁-C₆-alkyl, C₃-C₆-dihydroxycycloalkyl-C₁-C₆-haloalkylC₃-C₆-dihydroxycycloalkyl-C₂-C₆-alkenyl, C₃-C₆-dihydroxycycloalkyl-C₂-C₆-haloalkenyl, C₃-C₆-dihydroxycycloalkyl-C₂-C₆-alkynyl, C₃-C₆-dihydroxycycloalkyl-C₃-C₆-haloalkynyl, C₁-C₆-alkylcarbonyl-C₁-C₆-alkyl, C₁-C₆-haloalkylcarbonyl-C₁-C₆-alkyl, C₁-C₆-alkylcarbonyl-C₁-C₆-haloalkyl, C₁-C₆-haloalkylcarbonyl-C₁-C₆-haloalkyl, hydroxycarbonyl-C₂-C₆-alkenyl, hydroxycarbonyl-C₂-C₆-haloalkenyl, C₁-C₆-alkoxycarbonyl-C₂-C₆-alkenyl, C₁-C₆-haloalkoxycarbonyl-C₂-C₆-alkenyl, C₁-C₆-alkoxycarbonyl-C₂-C₆-haloalkenyl, C₁-C₆-haloalkoxycarbonyl-C₂-C₆-haloalkenyl, hydroxycarbonyl-C₂-C₆-alkynyl, hydroxycarbonyl-C₃-C₆-haloalkynyl, C₁-C₆-alkoxycarbonyl-C₂-C₆-alkynyl, C₁-C₆-haloalkoxycarbonyl-C₂-C₆-alkynyl, C₁-C₆-alkoxycarbonyl-C₃-C₆-haloalkynyl, C₁-C₆-haloalkoxycarbonyl-C₃-C₆-haloalkynyl, C₁-C₆-cyanoalkyl, C₂-C₆-cyanohaloalkyl, C₁-C₆-dicyanoalkyl, C₂-C₆-dicyanohaloalkyl, di(hydroxycarbonyl)-C₁-C₆-alkyl, di(hydroxycarbonyl)-C₁-C₆-haloalkyl, di(C₁-C₆-alkoxycarbonyl)-C₁-C₆-alkyl, di(C₁-C₆-haloalkoxycarbonyl)-C₁-C₆-alkyl, di(C₁-C₆-alkoxycarbonyl)-C₁-C₆-haloalkyl, di(C₁-C₆-haloalkoxycarbonyl)-C₁-C₆-haloalkyl, di(C₁-C₆-alkoxyl)phosphoryl-C₁-C₆-alkyl, di(C₁-C₆-haloalkoxyl)phosphoryl-C₁-C₆-alkyl, di(C₁-C₆-alkoxyl)phosphoryl-C₁-C₆-haloalkyl, di(C₁-C₆-haloalkoxyl)phosphoryl-C₁-C₆-haloalkyl, phosphoryl-C₁-C₆-alkyl, phosphoryl-C₁-C₆-haloalkyl, di[di(C₁-C₆-alkoxyl)phosphoryl-)]C₁-C₆-alkyl, di[di(C₁-C₆-haloalkoxyl)phosphoryl-)]C₁-C₆-alkyl, di[di(C₁-C₆-alkoxyl)phosphoryl-)]C₁-C₆-haloalkyl, di[di(C₁-C₆-haloalkoxyl)phosphoryl-)]C₁-C₆-haloalkyl, diphosphoryl-C₁-C₆-alkyl, diphosphoryl-C₁-C₆-haloalkyl, C₁-C₆-alkylthio-C₁-C₆-alkyl, C₁-C₆-haloalkylthio-C₁-C₆-alkyl, C₁-C₆-alkylthio-C₁-C₆-haloalkyl, C₁-C₆-haloalkylthio-C₁-C₆-haloalkyl, C₁-C₆-alkylsulfinly-C₁-C₆-alkyl, C₁-C₆-haloalkylsulfinly-C₁-C₆-alkyl, C₁-C₆-alkylsulfinly-C₁-C₆-haloalkyl, C₁-C₆-haloalkylsulfinly-C₁-C₆-haloalkyl, C₁-C₆-alkylsulfonyl-C₁-C₆-alkyl, C₁-C₆-haloalkylsulfonyl-C₁-C₆-alkyl, C₁-C₆-haloalkylsulfonyl-C₁-C₆-haloalkyl, phenyl, 5- or 6-membered heteroaryl, 3- to 6-membered heterocyclyl;
wherein hydroxy groups of R² are unsubstituted or substituted by R^{b};
cyclic groups of R² are unsubstituted or substituted by R^{c}; and
acyclic aliphatic groups of R² are unsubstituted or substituted by R^{d};
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
R^{c} is halogen, CN, NO₂, C₁-C₆-alkyl, C₁-C₆-haloalkyl, OH, C₁-C₆-alkoxy or C₁-C₆-haloalkoxy, C₁-C₆-alkylthio, C₁-C₆-alkylsulfinyl, or C₁-C₆-alkylsulfonyl;
R^{d} is phenyl, 5- or 6-membered heteroaryl, or 3- to 6-membered heterocyclyl;
wherein the substituent R^{d} is unsubstituted or substituted by R^{e};
R^{e} is halogen, CN, NO₂, C₁-C₆-alkyl, C₁-C₆-haloalkyl, OH, C₁-C₆-alkoxy or C₁-C₆-haloalkoxy, C₁-C₆-alkylsulfonyl;
A is CR³ or NR^{3A};
Z is a 5 or 6 membered heteroaryl ring comprising A;
R³ is halogen, CN, NO₂, CHO, C₁-C₆-alkyl, C₁-C₆-haloalkyl, C₁-C₆-alkylcarbonyl, C₃-C₆-alkenyl, C₃-C₆-haloalkenyl, C₂-C₆-alkynyl, C₃-C₆-haloalkynyl, C₁-C₆-alkoxy, C₁-C₆-haloalkoxy, C₃-C₆-alkenyloxy, C₃-C₆-haloalkenyloxy, C₃-C₆-alkynyloxy, C₃-C₆-haloalkynyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
wherein the cyclic groups of R³ are unsubstituted or substituted by substituents R^{a};
R^{3A} is H, C₁-C₆-alkyl, C₁-C₆-haloalkyl, C₁-C₆-alkylcarbonyl, C₃-C₆-alkenyl, C₃-C₆-haloalkenyl, C₂-C₆-alkynyl, C₃-C₆-haloalkynyl, C₁-C₆-alkoxy, C₁-C₆-haloalkoxy, C₃-C₆-alkenyloxy, C₃-C₆-haloalkenyloxy, C₃-C₆-alkynyloxy, C₃-C₆-haloalkynyloxy, C₁-C₆-alkoxy-C₁-C₆-alkoxy, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
wherein the cyclic groups of R^{3A} are unsubstituted or substituted by R^{a};
R⁴ is halogen, CN, NO₂, CHO, C₁-C₆-alkyl, C₁-C₆-haloalkyl, C₁-C₆-alkylcarbonyl, C₃-C₆-alkenyl, C₃-C₆-haloalkenyl, C₃-C₆-alkenyl, C₃-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
wherein the cyclic groups of R⁴ are unsubstituted or substituted by R^{a};
R^{a} is halogen, CN, NO₂, C₁-C₆-alkyl, C₁-C₆-haloalkyl, C₁-C₆-alkoxy, or C₁-C₆-haloalkoxy;
m is 0, 1, 2, or 3;
or an agriculturally acceptable salt or derivative of compound of formula (I) having an acidic functionality.

17. The pyrimidine compound of formula (I) of claim 16, wherein the heteroaryl ring Z is selected from rings A to G, 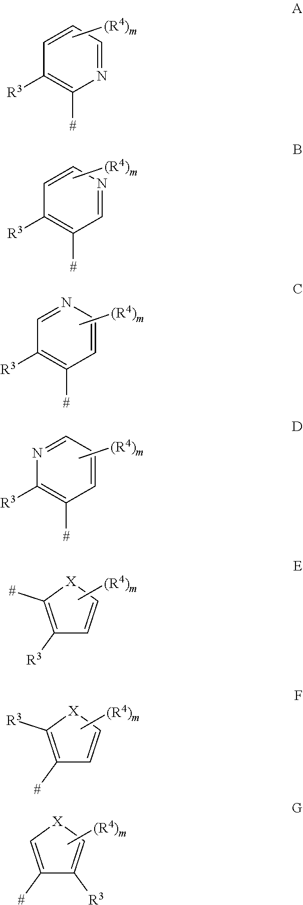
wherein
R³ is halogen, CHO, CN, C₁-C₆-alkyl, C₁-C₆-haloalkyl, or C₁-C₆-alkoxy;
m is 0 or 1;
R⁴ is halogen, CHO, CN, C₁-C₆-alkyl, C₁-C₆-haloalkyl, or C₁-C₆-alkoxy;
X is O, S, or NR^{3A};
R^{3A} is H, C₁-C₆-alkyl, C₁-C₆-haloalkyl, C₁-C₆-alkylcarbonyl, C₃-C₆-alkenyl, C₃-C₆-haloalkenyl, C₃-C₆-alkenyl, C₃-C₆-haloalkenyl, or C₃-C₆-cycloalkyl;
# denotes the point of attachment to the pyrimidine ring.

18. The pyrimidine compound of formula (I) of claim 16, wherein
R¹ is C₁-C₆-alkyl, C₁-C₆-alkoxy, C₁-C₆-haloalkoxy, C₃-C₆-alkenyloxy, C₃-C₆-haloalkenyloxy C₃-C₆-alkynyloxy, C₃-C₆-haloalkynyloxy, C₁-C₆-alkylthio, or C₃-C₆-cycloalkyl, wherein the cycloalkyl substituent is unsubstituted.

19. The pyrimidine compound of formula (I) of claim 16, wherein
R² is C₂-C₆-alkenyl, C₂-C₆-haloalkenyl, C₁-C₆-alkoxy-C₂-C₆-alkenyl, C₂-C₆-alkynyl, C₃-C₆-cycloalkenyl, C₃-C₆-cycloalkenyl-C₁-C₆-alkyl, C₃-C₆-cycloalkyl-C₁-C₆-alkylidenyl, C₃-C₆-halocycloalkyl-C₁-C₆-alkylidenyl, C₃-C₆-cycloalkenyl-C₁-C₆-alkylidenyl, C₃-C₆-hydroxycycloalkyl-C₁-C₆-alkyl, C₃-C₆-hydroxycycloalkenyl-C₁-C₆-alkyl, C₁-C₆-hydroxyalkyl, C₃-C₆-cycloalkyl-C₂-C₆-hydroxyalkylidenyl, hydroxycarbonyl-C₁-C₆-alkyl, hydroxycarbonyl-C₁-C₆-haloalkyl, C₁-C₆-alkoxycarbonyl-C₁-C₆-alkyl, C₃-C₆-hydroxycycloalkyl-C₁-C₆-hydroxyalkyl, C₂-C₆-dihydroxyalkyl, C₃-C₆-cycloalkyl-C₃-C₆-dihydroxyalkylidenyl, hydroxycarbonyl-C₂-C₆-dihydroxyalkyl, C₁-C₆-alkoxycarbonyl-C₂-C₆-dihydroxyalkyl, C₁-C₆-dicyanoalkyl, or 5- or 6-membered heteroaryl;
wherein OH groups of R² are unsubstituted or substituted by R^{b};
cyclic groups of R² are unsubstituted or substituted by R^{c}; and
acyclic aliphatic groups of R² are unsubstituted or substituted by R^{d};
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
R^{c} is halogen, CN, NO₂, C₁-C₆-alkyl, C₁-C₆-haloalkyl, OH, C₁-C₆-alkoxy or C₁-C₆-haloalkoxy, C₁-C₆-alkylthio, C₁-C₆-alkylsulfinyl, or C₁-C₆-alkylsulfonyl;
R^{d} is phenyl, 5- or 6-membered heteroaryl, or 3- to 6-membered heterocyclyl;
wherein the substituent R^{d} is unsubstituted or substituted by R^{e};
R^{e} is halogen, CN, NO₂, C₁-C₆-alkyl, C₁-C₆-haloalkyl, OH, C₁-C₆-alkoxy or C₁-C₆-haloalkoxy, C₁-C₆-alkylsulfonyl.

20. The pyrimidine compound of formula (I) of claim 16, wherein
R¹ is c-C₃H₅;
R² is C₁-C₆-alkyl, C₂-C₆-alkenyl, C₃-C₆-cycloalkyl-C₁-C₆-alkyl, C₃-C₆-cycloalkenyl-C₁-C₆-alkyl, C₃-C₆-cycloalkyl-C₁-C₆-alkylidenyl, C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-alkyl, C₁-C₆-alkoxycarbonyl-C₁-C₆-haloalkyl, or 5-membered heteroaryl;
wherein OH groups of R² are unsubstituted or substituted by R^{b},
cyclic groups of R² are unsubstituted or substituted by R^{c}, and
acyclic aliphatic groups of R² are unsubstituted or substituted by R^{d};
R^{b} is C₁-C₆-alkyl;
R^{e} is C₁-C₆-alkyl or OH;
R^{d} is phenyl or 5- or 6-membered heteroaryl;
wherein the substituent R^{d} is unsubstituted or substituted by R^{e};
R^{e} is halogen, CN, NO₂, C₁-C₆-alkyl, C₁-C₆-haloalkyl, OH, C₁-C₆-alkoxy, C₁-C₆-haloalkoxy, C₁-C₆-alkylsulfonyl;
Z is A, E, F, or G;
X is S;
R³ is Cl, Br, F, I, CH₃, or OCF₃;
m is 0 or 1;
R⁴ is Br.

21. A herbicidal composition comprising:
A) at least one pyrimidine compound of formula I, or an agriculturally acceptable salt or derivative of compound of formula (I) having an acidic functionality, of claim 16;
and
B) herbicides of class b1) to b15):
b1) lipid biosynthesis inhibitors;
b2) acetolactate synthase inhibitors (ALS inhibitors);
b3) photosynthesis inhibitors;
b4) protoporphyrinogen-IX oxidase inhibitors,
b5) bleacher herbicides;
b6) enolpyruvyl shikimate 3-phosphate synthase inhibitors (EPSP inhibitors);
b7) glutamine synthetase inhibitors;
b8) 7,8-dihydropteroate synthase inhibitors (DHP inhibitors);
b9) mitosis inhibitors;
b10) inhibitors of the synthesis of very long chain fatty acids (VLCFA inhibitors);
b11) cellulose biosynthesis inhibitors;
b12) decoupler herbicides;
b13) auxinic herbicides;
b14) auxin transport inhibitors; and
b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol, and its salts and esters;
or an agriculturally acceptable salt or derivative thereof.

22. A herbicidal composition comprising the herbicidal composition of claim 21, and safeners.

23. The herbicidal composition of claim 21, wherein the herbicidal composition comprises at least one herbicide B selected from herbicides of class b1, b2, b3, b4, b5, b6, b9, b10, b13 and b14.

24. The herbicidal composition of claim 21, wherein the herbicidal composition comprises at least one herbicide B selected from herbicides of class b1, b2, b4, b5, b9, b10, b13 and b14.

25. The herbicidal composition of claim 21, wherein the weight ratio of component A to component B is in the range of from 1:500 to 500:1.

26. A herbicidal composition comprising a herbicidal active amount of at least one pyrimidine compound of formula (I) or an agriculturally acceptable salt or derivative of compound of formula (I) having an acidic functionality, of claim 16, and at least one inert liquid and/or solid carrier and, if appropriate, at least one surface-active substance.

27. A herbicidal composition comprising a herbicidal composition of claim 21, and at least one inert liquid and/or solid carrier and, if appropriate, at least one surface-active substance.

28. A method of controlling undesired vegetation, which comprises allowing a herbicidal active amount of a composition of claim 21 to act on plants, their environment or on seed.

29. A method of controlling undesired vegetation, which comprises allowing a herbicidal active amount of at least one pyrimidine compound of formula (I) or an agriculturally acceptable salt or derivative of compound of formula (I) having an acidic functionality, of claim 16 to act on plants, their environment or on seed.

30. The method of claim 29, wherein the heteroaryl ring Z is selected from rings A to G, 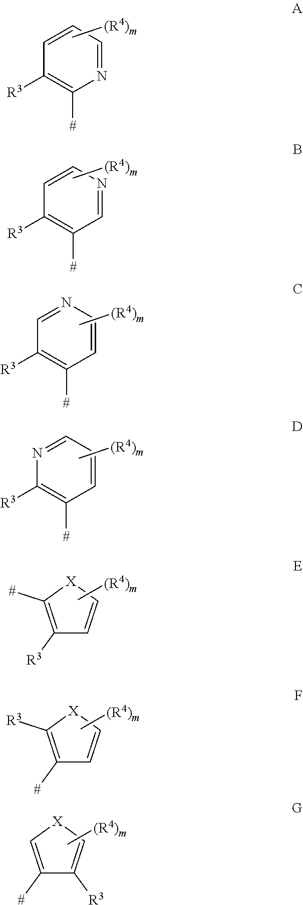
wherein
R³ is halogen, CHO, CN, C₁-C₆-alkyl, C₁-C₆-haloalkyl, or C₁-C₆-alkoxy;
m is 0 or 1;
R⁴ is halogen, CHO, CN, C₁-C₆-alkyl, C₁-C₆-haloalkyl, or C₁-C₆-alkoxy;
X is O, S, or NR^{3A};
R^{3A} is H, C₁-C₆-alkyl, C₁-C₆-haloalkyl, C₁-C₆-alkylcarbonyl, C₃-C₆-alkenyl, C₃-C₆-haloalkenyl, C₃-C₆-alkenyl, C₃-C₆-haloalkenyl, or C₃-C₆-cycloalkyl;
# denotes the point of attachment to the pyrimidine ring.

31. The method of claim 29, wherein
R¹ is C₁-C₆-alkyl, C₁-C₆-alkoxy, C₁-C₆-haloalkoxy, C₃-C₆-alkenyloxy, C₃-C₆-haloalkenyloxy C₃-C₆-alkynyloxy, C₃-C₆-haloalkynyloxy, C₁-C₆-alkylthio, or C₃-C₆-cycloalkyl, wherein the cycloalkyl substituent is unsubstituted.

32. The method of claim 29, wherein
R² is C₂-C₆-alkenyl, C₂-C₆-haloalkenyl, C₁-C₆-alkoxy-C₂-C₆-alkenyl, C₂-C₆-alkynyl, C₃-C₆-cycloalkenyl, C₃-C₆-cycloalkenyl-C₁-C₆-alkyl, C₃-C₆-cycloalkyl-C₁-C₆-alkylidenyl, C₃-C₆-halocycloalkyl-C₁-C₆-alkylidenyl, C₃-C₆-cycloalkenyl-C₁-C₆-alkylidenyl, C₃-C₆-hydroxycycloalkyl-C₁-C₆-alkyl, C₃-C₆-hydroxycycloalkenyl-C₁-C₆-alkyl, C₁-C₆-hydroxyalkyl, C₃-C₆-cycloalkyl-C₂-C₆-hydroxyalkylidenyl, hydroxycarbonyl-C₁-C₆-alkyl, hydroxycarbonyl-C₁-C₆-haloalkyl, C₁-C₆-alkoxycarbonyl-C₁-C₆-alkyl, C₃-C₆-hydroxycycloalkyl-C₁-C₆-hydroxyalkyl, C₂-C₆-dihydroxyalkyl, C₃-C₆-cycloalkyl-C₃-C₆-dihydroxyalkylidenyl, hydroxycarbonyl-C₂-C₆-dihydroxyalkyl, C₁-C₆-alkoxycarbonyl-C₂-C₆-dihydroxyalkyl, C₁-C₆-dicyanoalkyl, or 5- or 6-membered heteroaryl;
wherein OH groups of R² are unsubstituted or substituted by R^{b};
cyclic groups of R² are unsubstituted or substituted by R^{c}; and
acyclic aliphatic groups of R² are unsubstituted or substituted by R^{d};
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
R^{c} is halogen, CN, NO₂, C₁-C₆-alkyl, C₁-C₆-haloalkyl, OH, C₁-C₆-alkoxy or C₁-C₆-haloalkoxy, C₁-C₆-alkylthio, C₁-C₆-alkylsulfinyl, or C₁-C₆-alkylsulfonyl;
R^{d} is phenyl, 5- or 6-membered heteroaryl, or 3- to 6-membered heterocyclyl;
wherein the substituent R^{d} is unsubstituted or substituted by R^{e};
R^{e} is halogen, CN, NO₂, C₁-C₆-alkyl, C₁-C₆-haloalkyl, OH, C₁-C₆-alkoxy or C₁-C₆-haloalkoxy, C₁-C₆-alkylsulfonyl.

33. The method of claim 29, wherein
R¹ is c-C₃H₉;
R² is C₁-C₆-alkyl, C₂-C₆-alkenyl, C₃-C₆-cycloalkyl-C₁-C₆-alkyl, C₃-C₆-cycloalkenyl-C₁-C₆-alkyl, C₃-C₆-cycloalkyl-C₁-C₆-alkylidenyl, C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-alkyl, C₁-C₆-alkoxycarbonyl-C₁-C₆-haloalkyl, or 5-membered heteroaryl;
wherein OH groups of R² are unsubstituted or substituted by R^{b}, cyclic groups of R² are unsubstituted or substituted by R^{c}, and acyclic aliphatic groups of R² are unsubstituted or substituted by R^{d};
R^{b} is C₁-C₆-alkyl;
R^{c} is C₁-C₆-alkyl or OH;
R^{d} is phenyl or 5- or 6-membered heteroaryl;
wherein the substituent R^{d} is unsubstituted or substituted by R^{e};
R^{e} is halogen, CN, NO₂, C₁-C₆-alkyl, C₁-C₆-haloalkyl, OH, C₁-C₆-alkoxy, C₁-C₆-haloalkoxy, C₁-C₆-alkylsulfonyl;
Z is A, E, F, or G;
X is S;
R³ is Cl, Br, F, I, CH3, or OCF₃;
m is 0 or 1;
R⁴ is Br.
